(19) United States
(12) Patent Application Publication
(10) Pub. No.: US 2025/0017034 A1
(43) Pub. Date: Jan. 9, 2025

(54) LIGHT-EMITTING ELEMENT, DISPLAY DEVICE, AND AMINE COMPOUND FOR THE LIGHT-EMITTING ELEMENT

(71) Applicant: Samsung Display Co., Ltd., Yongin-si (KR)

(72) Inventors: CHAEYEONG KIM, Yongin-si (KR); MINJI KIM, Yongin-si (KR); BEOMJOON KIM, Yongin-si (KR); BYEONGWOOK YOO, Yongin-si (KR); JEONG MIN LEE, Yongin-si (KR); EUNJAE JEONG, Yongin-si (KR); SOHEE JO, Yongin-si (KR); HAN NA CHA, Yongin-si (KR); SANGHYUN HAN, Yongin-si (KR)

(21) Appl. No.: 18/662,976

(22) Filed: May 13, 2024

(30) Foreign Application Priority Data
Jun. 22, 2023 (KR) 10-2023-0080381

(51) Int. Cl. H10K 50/12; H10K 50/155; H10K 85/30; H10K 85/40; H10K 85/60

(52) U.S. Cl. CPC H10K 50/12; H10K 50/155; H10K 85/342; H10K 85/346; H10K 85/40; H10K 85/615; H10K 85/633; H10K 85/636; H10K 85/654; H10K 85/6572; H10K 85/6574; H10K 85/6576

(57) ABSTRACT

A light-emitting element may include a first electrode, a second electrode disposed on the first electrode, and at least one functional layer disposed between the first electrode and the second electrode and containing an amine compound represented by Formula 1.

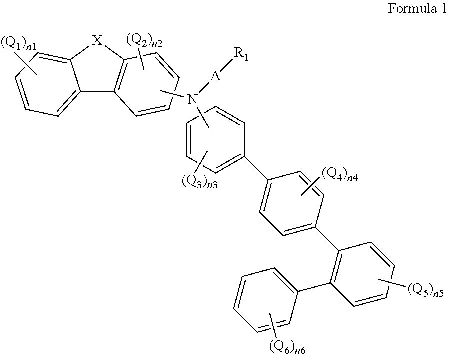

### CROSS-REFERENCE TO RELATED APPLICATIONS

The present application claims priority to and the benefit of Korean Patent Application No. 10-2023-0080381, filed on Jun. 22, 2023, in the Korean Intellectual Property Office, the entire content of which is incorporated herein by reference.

### BACKGROUND

### 1. Field

Embodiments of the present disclosure described herein are related to a light-emitting element, a display device, and an amine compound utilized for the light-emitting element.

### 2. Description of the Related Art

Recently, organic electroluminescence display devices have been actively developed as image display devices. Organic electroluminescence display devices are different from liquid crystal display devices and are self-luminous type or kind display devices. For example, an organic electroluminescence display device recombines, in its light-emitting layer, holes and electrons (e.g., respectively or separately) injected from its first electrode and its second electrode, thereby causing a light-emitting material containing an organic compound, of the light-emitting layer, to emit light to implement displays (e.g., images).

In applying an organic electroluminescence element to display devices, improvements in low driving voltage, high luminous efficiency, and/or long-lifespan of the organic electroluminescence element are required/or desired, and thus the material development for an organic electroluminescence element capable of stably achieving such improvements, is continuously required and/or pursued.

In some embodiments, to achieve highly efficient organic electroluminescence elements, materials for a hole transport layer are under development.

### SUMMARY

Aspects according to one or more embodiments of the present disclosure are directed toward a light-emitting element having improved luminous efficiency and/or element lifespan.

Aspects according to one or more embodiments of the present disclosure are directed toward a display device having improved luminous efficiency.

Aspects according to one or more embodiments of the present disclosure are directed toward an amine compound capable of improving luminous efficiency and/or element lifespan of a light-emitting element.

Additional aspects will be set forth in part in the description which follows and, in part, will be apparent from the description, or may be learned by practice of the presented embodiments of the present disclosure.

According to one or more embodiments of the present disclosure, a light-emitting element may include a first electrode, a second electrode facing the first electrode (e.g., the second electrode is spaced from the first electrode), and a functional layer disposed between the first electrode and the second electrode, wherein the functional layer contains an amine compound represented by Formula 1.

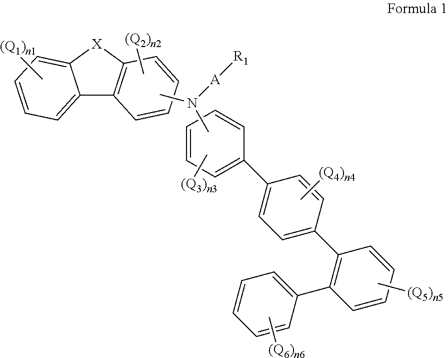

wherein, in Formula 1 above, n1, n3, n4, and n5 may each independently be an integer of 0 to 4, n2 is an integer of 0 to 3, n6 is an integer of 0 to 5, n1 and n2 are not 0 at the same time, X is O, S, NR₂, or CR₃R₄, A is a substituted or unsubstituted arylene group having 6 to 30 ring-forming carbon atoms, R₁ is a substituted or unsubstituted cycloalkyl group having 3 to 10 ring-forming carbon atoms, R₂, R₃, and R₄ may each independently be a hydrogen atom, a deuterium atom, a substituted or unsubstituted alkyl group having 1 to 30 carbon atoms, or a substituted or unsubstituted aryl group having 6 to 30 ring-forming carbon atoms, Q₁ and Q₂ may each independently be a substituted or unsubstituted alkyl group having 1 to 10 carbon atoms, or a substituted or unsubstituted aryl group having 6 to 30 ring-forming carbon atoms, and Q₃, Q₄, Q₅, and Q₆ may each independently be a hydrogen atom, a deuterium atom, a substituted or unsubstituted alkyl group having 1 to 10 carbon atoms, or a substituted or unsubstituted aryl group having 6 to 30 ring-forming carbon atoms.

According to one or more embodiments, the functional layer may include a light-emitting layer, a hole transport region disposed between the first electrode and the light-emitting layer, and an electron transport region disposed between the light-emitting layer and the second electrode, and the hole transport region may contain the amine compound represented by Formula 1 above.

According to one or more embodiments, the hole transport region may include a hole injection layer disposed between the first electrode and the light-emitting layer, and a hole transport layer disposed between the hole injection layer and the light-emitting layer, and at least any one selected from among the hole injection layer and the hole transport layer may contain the amine compound represented by Formula 1 above.

According to one or more embodiments, the hole transport region may further include an electron-blocking layer disposed between the hole transport layer and the light-emitting layer, and at least any one selected from among the hole injection layer, the hole transport layer, and the electron-blocking layer may contain the amine compound represented by Formula 1 above.

According to one or more embodiments, the amine compound represented by Formula 1 above may be a monoamine compound.

According to one or more embodiments, the amine compound represented by Formula 1 above may be represented by Formula 1-1.

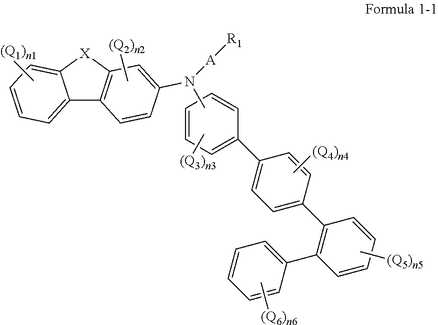

wherein, in Formula 1-1 above, n1 to n6, X, A, R₁ to R₄, and Q₁ to Q₆ may each independently be the same as defined in the descriptions of Formula 1.

In an embodiment, the amine compound represented by Formula 1 above may be represented by any one selected from among Formulas 2-1 to 2-3.

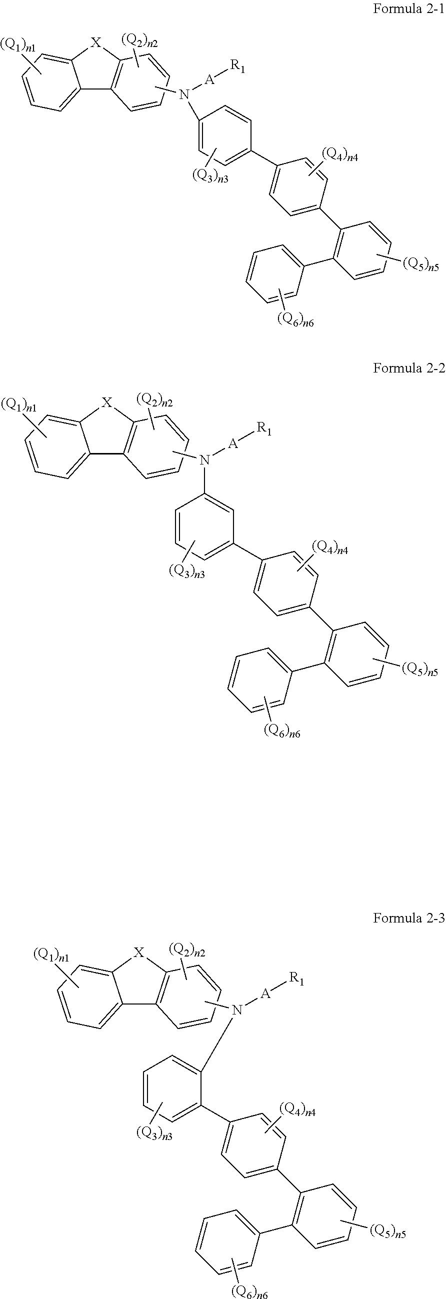

wherein, in Formulas 2-1 to 2-3 above, n1 to n6, X, A, R₁ to R₄, and Q₁ to Q₆ may each independently be the same as defined in the descriptions of Formula 1.

According to one or more embodiments, the amine compound represented by Formula 1 above may be represented by any one selected from among Formulas 3-1 to 3-4.

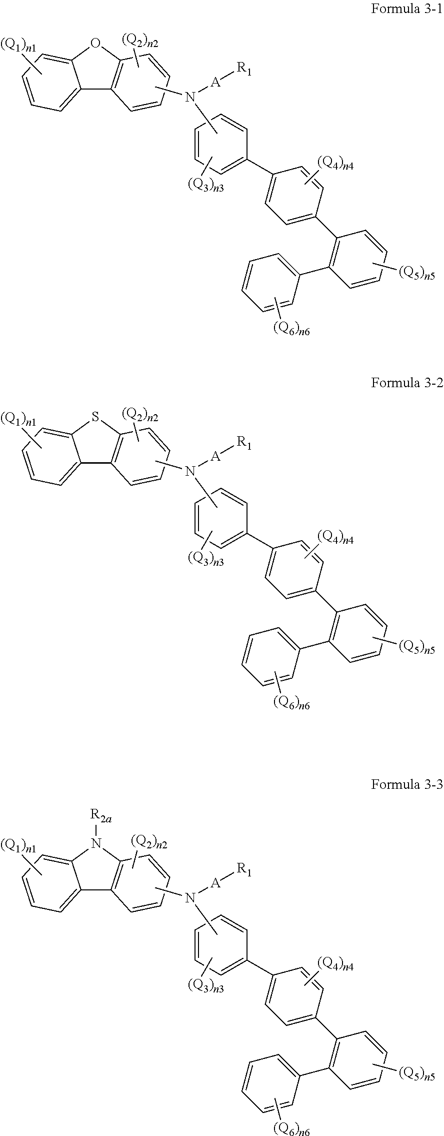

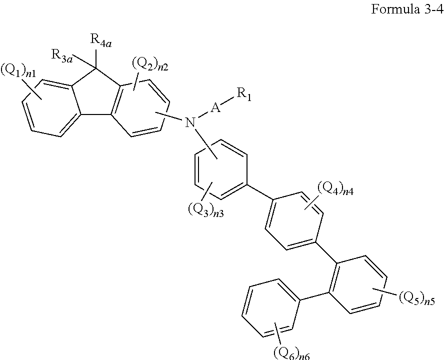

wherein, in Formulas 3-1 to 3-4 above, R₂ₐ, R₃ₐ, and R₄ₐ may each independently be a hydrogen atom, a deuterium atom, an unsubstituted alkyl group having 1 to 30 carbon atoms, or an unsubstituted aryl group having 6 to 30 ring-forming carbon atoms, and n1 to n6, A, R₁, and Q₁ to Q₆ each may each independently be the same as defined in the descriptions of Formula 1 above.

According to one or more embodiments, the amine compound represented by Formula 1 above may be represented by Formula 4.

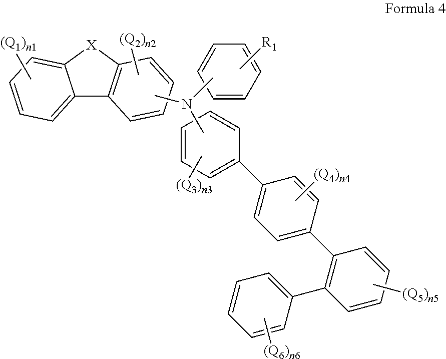

wherein, in Formula 4 above, n1 to n6, X, R₁ to R₄, and Q₁ to Q₆ may each independently be the same as defined in the descriptions of Formula 1.

According to one or more embodiments, the amine compound represented by Formula 4 above may be represented by Formula 4-1.

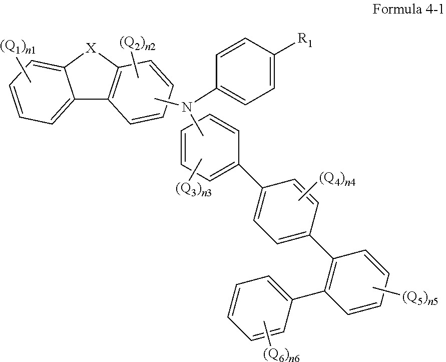

wherein, in Formula 4-1 above, n1 to n6, X, R₁ to R₄, and Q₁ to Q₆ may each independently be the same as defined in the descriptions of Formula 1 above.

According to one or more embodiments, in Formula 1 above, R₁ may be a substituted or unsubstituted cyclohexyl group, a substituted or unsubstituted bicycloheptanyl group, or a substituted or unsubstituted adamantly group.

According to one or more embodiments, the amine compound represented by Formula 1 above may be represented by Formula 5.

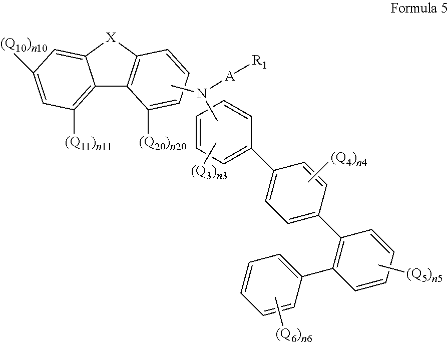

wherein, in Formula 5 above, n10, n11 and n20 may each independently be 0 or 1, n10, n11, and n20 are not 0 at the same time, Q₁ is a substituted or unsubstituted alkyl group having 1 to 10 carbon atoms, Q₁₁ and Q₁₂ may each independently be alkyl group having 1 to 10 carbon atoms, or an unsubstituted aryl group having 6 to 30 ring-forming carbon atoms, and n3 to n6, X, A, R₁ to R₄, and Q₃ to Q₆ may each independently be the same as defined in the descriptions of Formula 1 above.

According to one or more embodiments, in Formula 5, Q₁ may be a substituted or unsubstituted methyl group, and Q₁₁ and Q₂₀ may each independently be a substituted or unsubstituted methyl group or a substituted or unsubstituted phenyl group.

According to one or more embodiments, the amine compound represented by Formula 1 above may be represented by any one selected from among compounds presented in Compound Group 1.

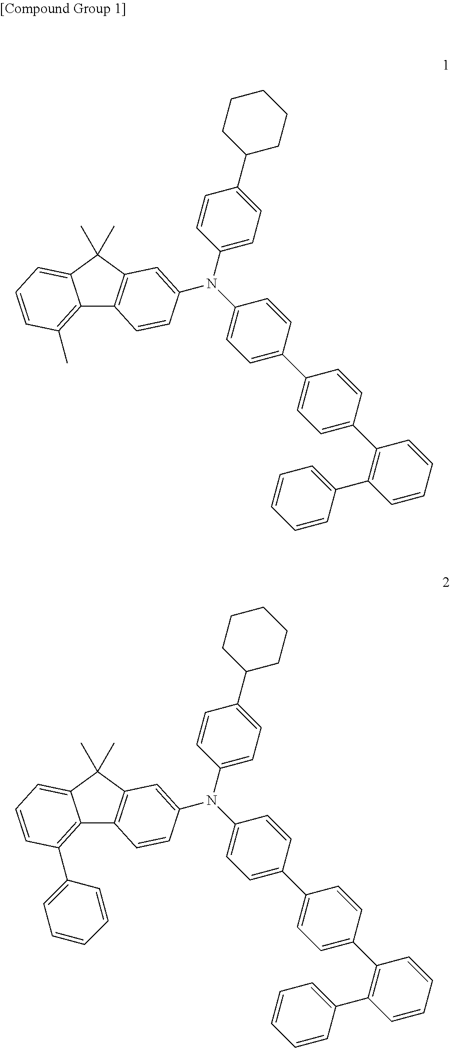 [SMILES: Cc1cccc2c1-c1ccc(N(c3ccc(-c4ccc(-c5ccccc5-c5ccccc5)cc4)cc3)c3ccc(C4CCCCC4)cc3)cc1C2(C)C ; CC1(C)c2cc(N(c3ccc(-c4ccc(-c5ccccc5-c5ccccc5)cc4)cc3)c3ccc(C4CCCCC4)cc3)ccc2-c2c(-c3ccccc3)cccc21]

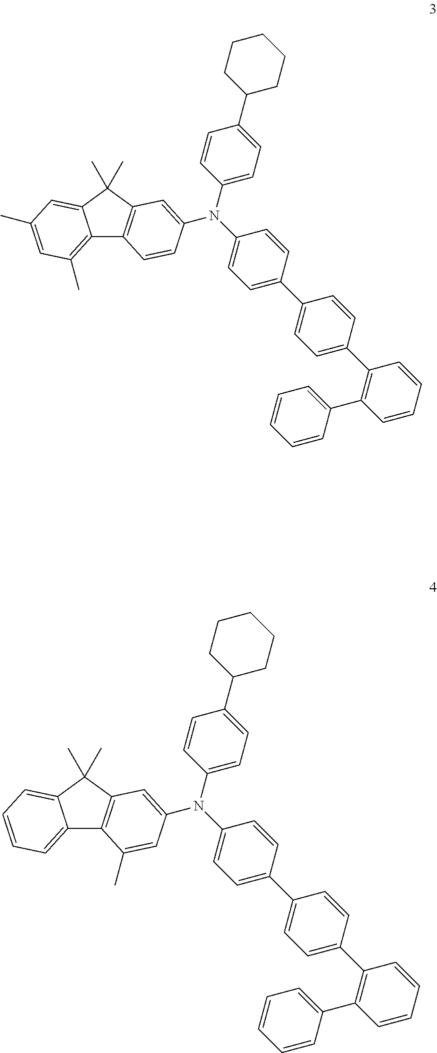 [SMILES: Cc1cc(C)c2c(c1)C(C)(C)c1cc(N(c3ccc(-c4ccc(-c5ccccc5-c5ccccc5)cc4)cc3)c3ccc(C4CCCCC4)cc3)ccc1-2 ; Cc1cc(N(c2ccc(-c3ccc(-c4ccccc4-c4ccccc4)cc3)cc2)c2ccc(C3CCCCC3)cc2)cc2c1-c1ccccc1C2(C)C]

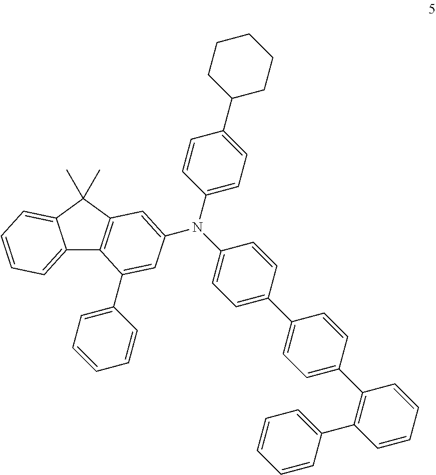 [SMILES: CC1(C)c2ccccc2-c2c(-c3ccccc3)cc(N(c3ccc(-c4ccc(-c5ccccc5-c5ccccc5)cc4)cc3)c3ccc(C4CCCCC4)cc3)cc21]

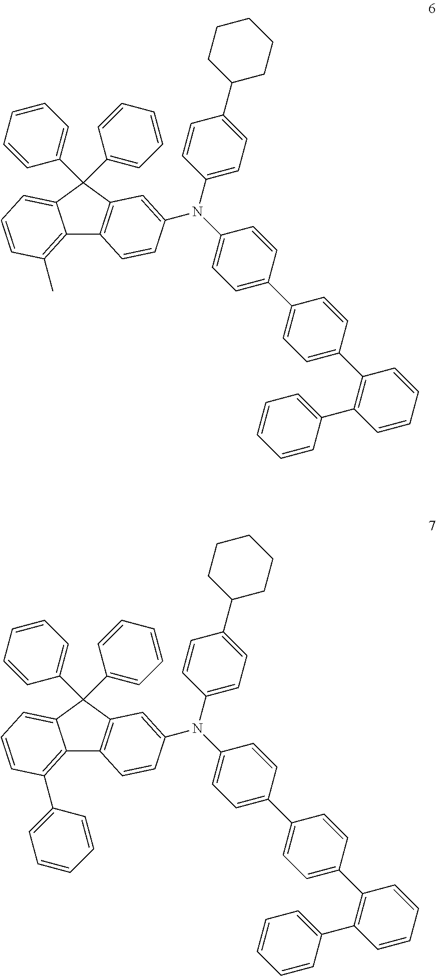 [SMILES: Cc1cccc2c1-c1ccc(N(c3ccc(-c4ccc(-c5ccccc5-c5ccccc5)cc4)cc3)c3ccc(C4CCCCC4)cc3)cc1C2(c1ccccc1)c1ccccc1 ; c1ccc(-c2ccccc2-c2ccc(-c3ccc(N(c4ccc(C5CCCCC5)cc4)c4ccc5c(c4)C(c4ccccc4)(c4ccccc4)c4cccc(-c6ccccc6)c4-5)cc3)cc2)cc1]

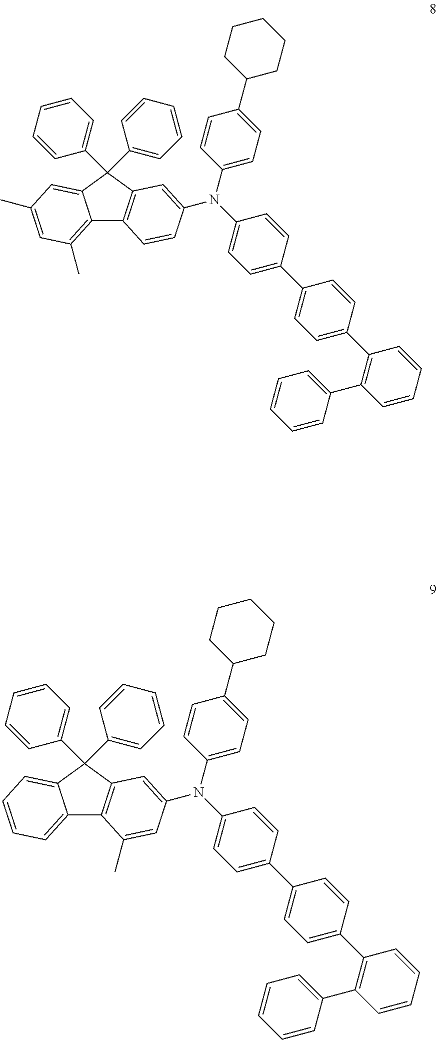 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C4CCCCC4)cc3)ccc1-2 ; Cc1cc(N(c2ccc(-c3ccc(-c4ccccc4-c4ccccc4)cc3)cc2)c2ccc(C3CCCCC3)cc2)cc2c1-c1ccccc1C2(c1ccccc1)c1ccccc1]

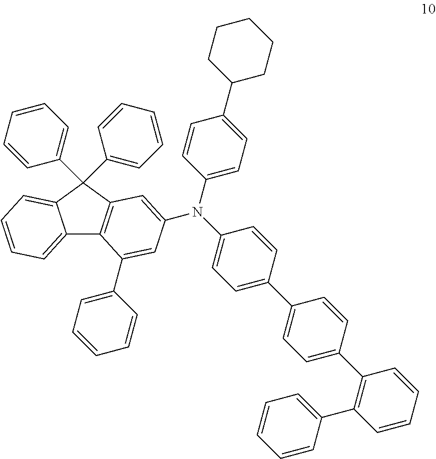 [SMILES: c1ccc(-c2ccccc2-c2ccc(-c3ccc(N(c4ccc(C5CCCCC5)cc4)c4cc(-c5ccccc5)c5c(c4)C(c4ccccc4)(c4ccccc4)c4ccccc4-5)cc3)cc2)cc1]

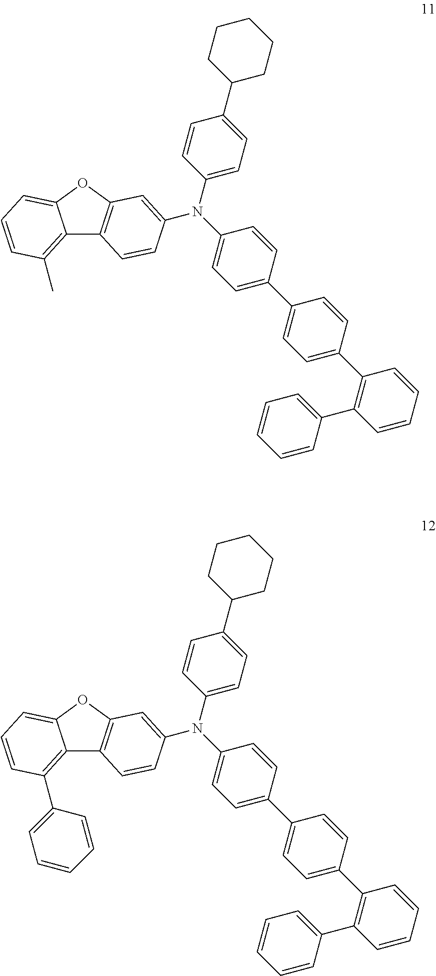 [SMILES: Cc1cccc2oc3cc(N(c4ccc(-c5ccc(-c6ccccc6-c6ccccc6)cc5)cc4)c4ccc(C5CCCCC5)cc4)ccc3c12 ; c1ccc(-c2ccccc2-c2ccc(-c3ccc(N(c4ccc(C5CCCCC5)cc4)c4ccc5c(c4)oc4cccc(-c6ccccc6)c45)cc3)cc2)cc1]

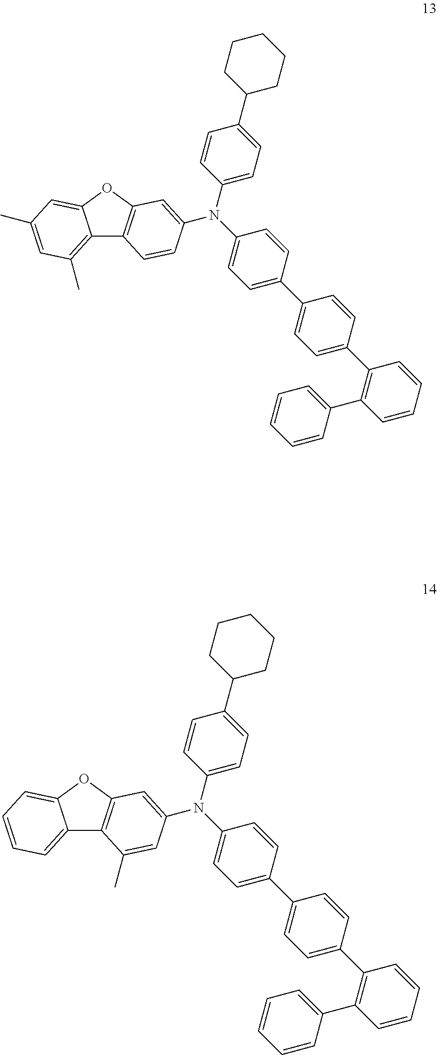 [SMILES: Cc1cc(C)c2c(c1)oc1cc(N(c3ccc(-c4ccc(-c5ccccc5-c5ccccc5)cc4)cc3)c3ccc(C4CCCCC4)cc3)ccc12 ; Cc1cc(N(c2ccc(-c3ccc(-c4ccccc4-c4ccccc4)cc3)cc2)c2ccc(C3CCCCC3)cc2)cc2oc3ccccc3c12]

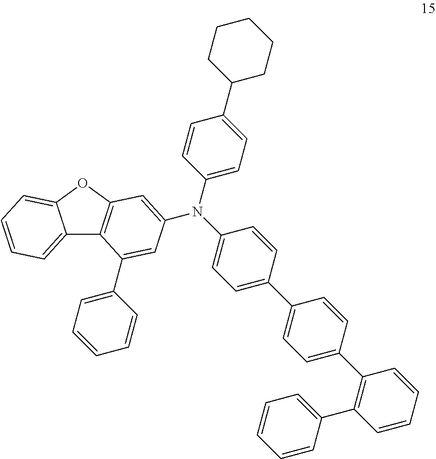 [SMILES: c1ccc(-c2ccccc2-c2ccc(-c3ccc(N(c4ccc(C5CCCCC5)cc4)c4cc(-c5ccccc5)c5c(c4)oc4ccccc45)cc3)cc2)cc1]

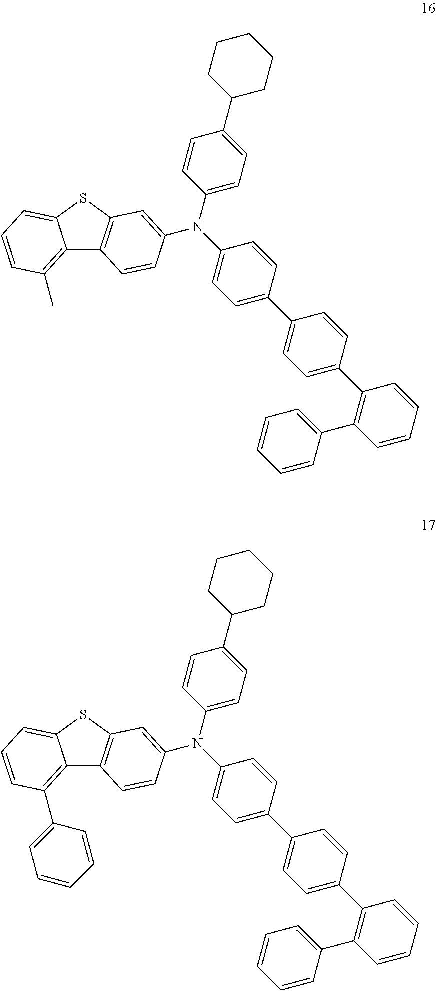 [SMILES: Cc1cccc2sc3cc(N(c4ccc(-c5ccc(-c6ccccc6-c6ccccc6)cc5)cc4)c4ccc(C5CCCCC5)cc4)ccc3c12 ; c1ccc(-c2ccccc2-c2ccc(-c3ccc(N(c4ccc(C5CCCCC5)cc4)c4ccc5c(c4)sc4cccc(-c6ccccc6)c45)cc3)cc2)cc1]

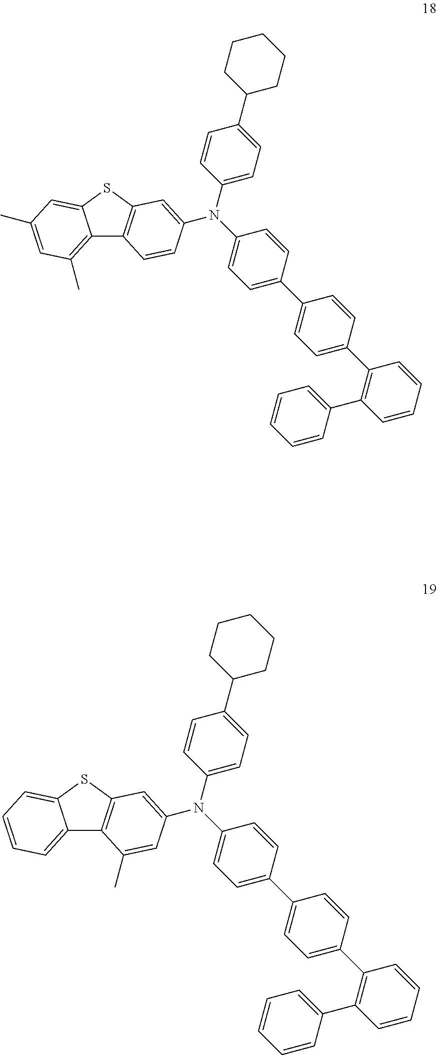 [SMILES: Cc1cc(C)c2c(c1)sc1cc(N(c3ccc(-c4ccc(-c5ccccc5-c5ccccc5)cc4)cc3)c3ccc(C4CCCCC4)cc3)ccc12 ; Cc1cc(N(c2ccc(-c3ccc(-c4ccccc4-c4ccccc4)cc3)cc2)c2ccc(C3CCCCC3)cc2)cc2sc3ccccc3c12]

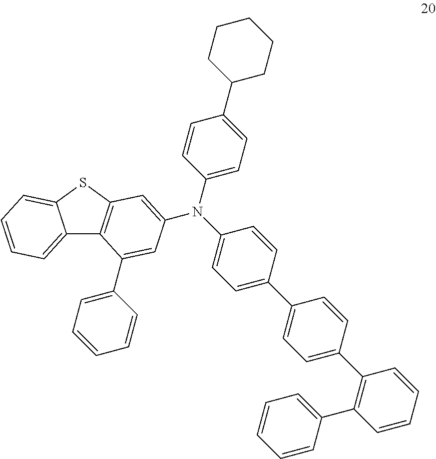 [SMILES: c1ccc(-c2ccccc2-c2ccc(-c3ccc(N(c4ccc(C5CCCCC5)cc4)c4cc(-c5ccccc5)c5c(c4)sc4ccccc45)cc3)cc2)cc1]

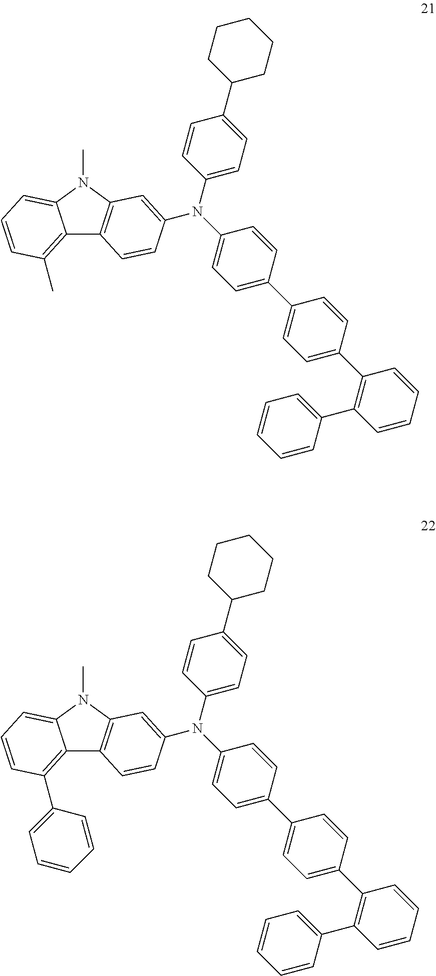 [SMILES: Cc1cccc2c1c1ccc(N(c3ccc(-c4ccc(-c5ccccc5-c5ccccc5)cc4)cc3)c3ccc(C4CCCCC4)cc3)cc1n2C ; Cn1c2cc(N(c3ccc(-c4ccc(-c5ccccc5-c5ccccc5)cc4)cc3)c3ccc(C4CCCCC4)cc3)ccc2c2c(-c3ccccc3)cccc21]

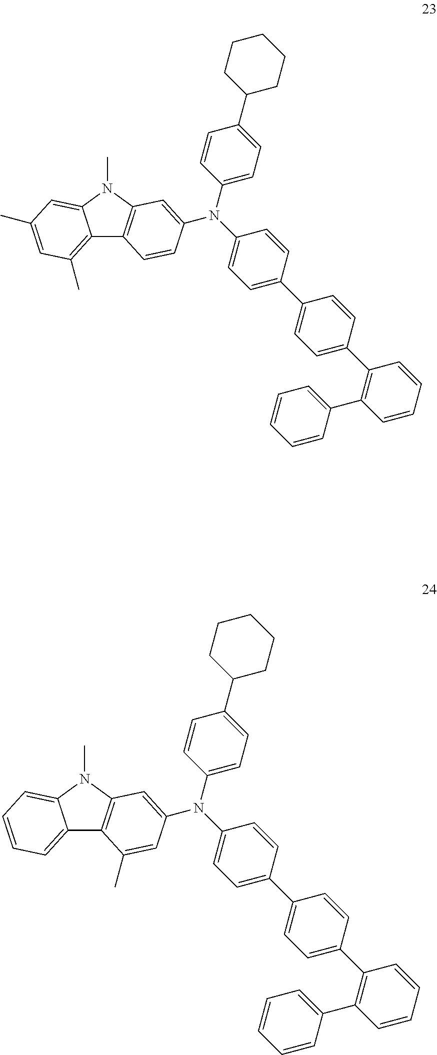 [SMILES: Cc1cc(C)c2c3ccc(N(c4ccc(-c5ccc(-c6ccccc6-c6ccccc6)cc5)cc4)c4ccc(C5CCCCC5)cc4)cc3n(C)c2c1 ; Cc1cc(N(c2ccc(-c3ccc(-c4ccccc4-c4ccccc4)cc3)cc2)c2ccc(C3CCCCC3)cc2)cc2c1c1ccccc1n2C]

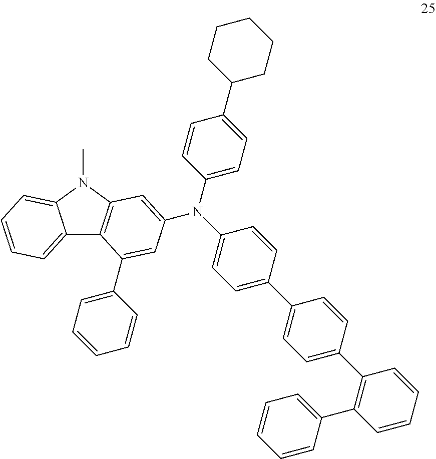 [SMILES: Cn1c2ccccc2c2c(-c3ccccc3)cc(N(c3ccc(-c4ccc(-c5ccccc5-c5ccccc5)cc4)cc3)c3ccc(C4CCCCC4)cc3)cc21]

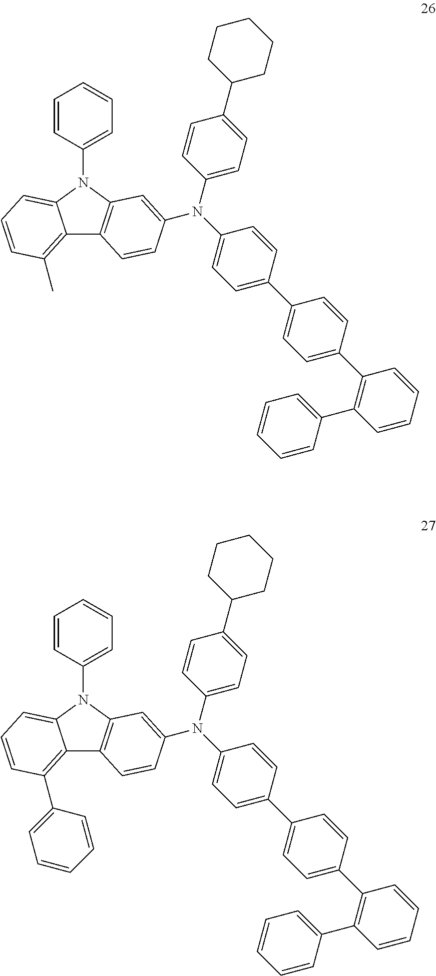 [SMILES: Cc1cccc2c1c1ccc(N(c3ccc(-c4ccc(-c5ccccc5-c5ccccc5)cc4)cc3)c3ccc(C4CCCCC4)cc3)cc1n2-c1ccccc1 ; c1ccc(-c2ccccc2-c2ccc(-c3ccc(N(c4ccc(C5CCCCC5)cc4)c4ccc5c6c(-c7ccccc7)cccc6n(-c6ccccc6)c5c4)cc3)cc2)cc1]

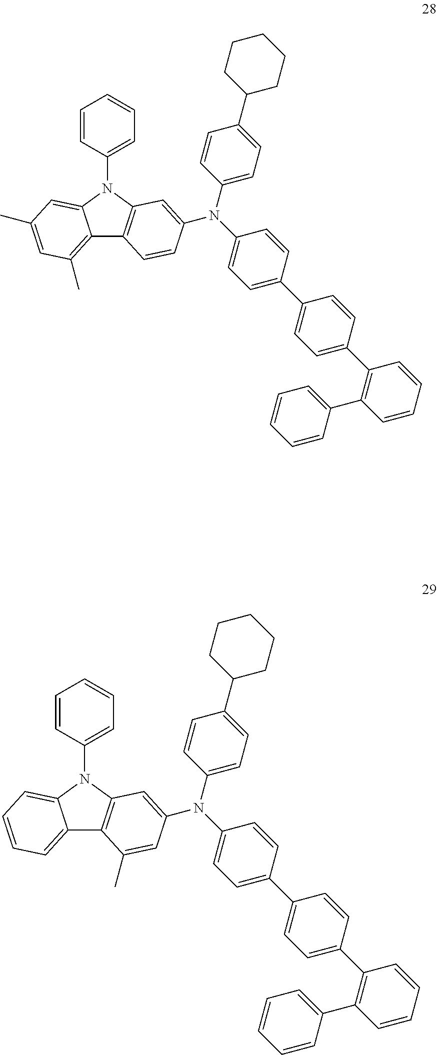 [SMILES: Cc1cc(C)c2c3ccc(N(c4ccc(-c5ccc(-c6ccccc6-c6ccccc6)cc5)cc4)c4ccc(C5CCCCC5)cc4)cc3n(-c3ccccc3)c2c1 ; Cc1cc(N(c2ccc(-c3ccc(-c4ccccc4-c4ccccc4)cc3)cc2)c2ccc(C3CCCCC3)cc2)cc2c1c1ccccc1n2-c1ccccc1]

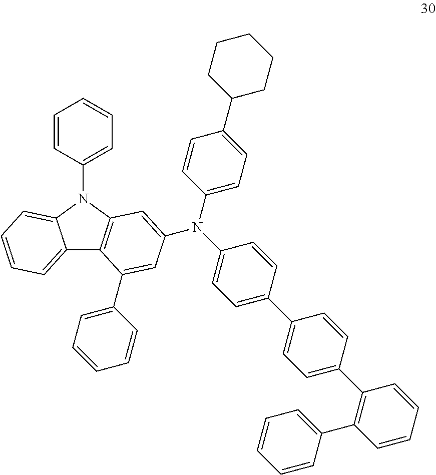 [SMILES: c1ccc(-c2ccccc2-c2ccc(-c3ccc(N(c4ccc(C5CCCCC5)cc4)c4cc(-c5ccccc5)c5c6ccccc6n(-c6ccccc6)c5c4)cc3)cc2)cc1]

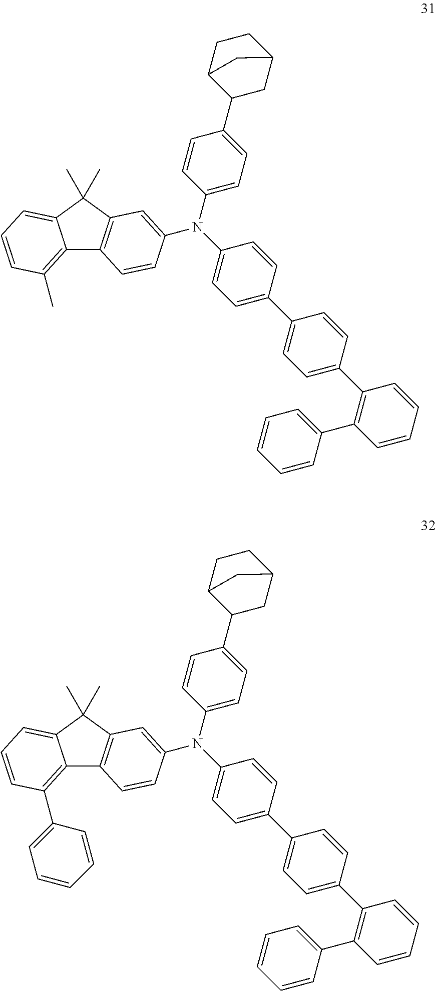 [SMILES: Cc1cccc2c1-c1ccc(N(c3ccc(-c4ccc(-c5ccccc5-c5ccccc5)cc4)cc3)c3ccc(C4CC5CCC4C5)cc3)cc1C2(C)C ; CC1(C)c2cc(N(c3ccc(-c4ccc(-c5ccccc5-c5ccccc5)cc4)cc3)c3ccc(C4CC5CCC4C5)cc3)ccc2-c2c(-c3ccccc3)cccc21]

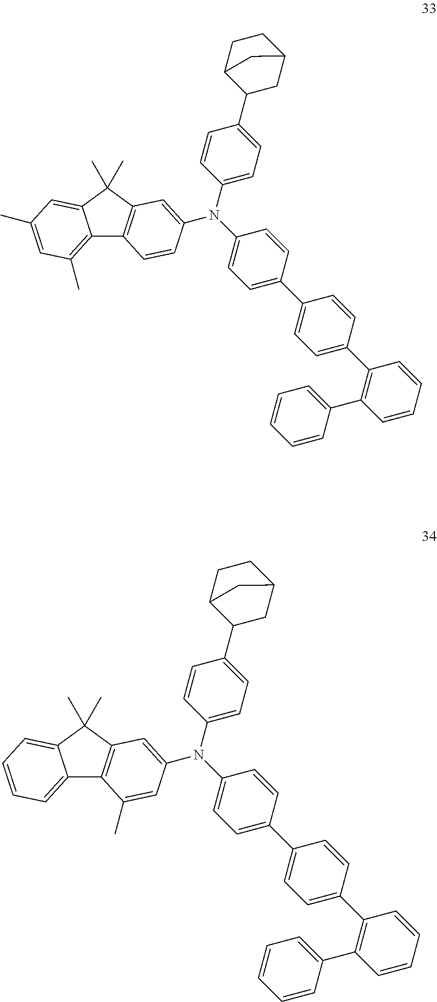 [SMILES: Cc1cc(C)c2c(c1)C(C)(C)c1cc(N(c3ccc(-c4ccc(-c5ccccc5-c5ccccc5)cc4)cc3)c3ccc(C4CC5CCC4C5)cc3)ccc1-2 ; Cc1cc(N(c2ccc(-c3ccc(-c4ccccc4-c4ccccc4)cc3)cc2)c2ccc(C3CC4CCC3C4)cc2)cc2c1-c1ccccc1C2(C)C]

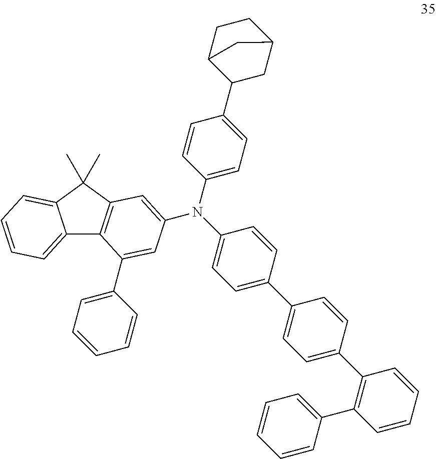 [SMILES: CC1(C)c2ccccc2-c2c(-c3ccccc3)cc(N(c3ccc(-c4ccc(-c5ccccc5-c5ccccc5)cc4)cc3)c3ccc(C4CC5CCC4C5)cc3)cc21]

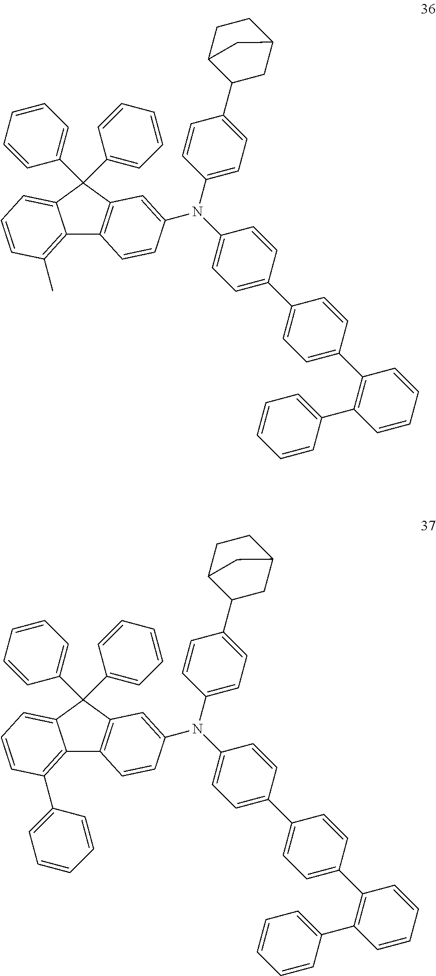 [SMILES: Cc1cccc2c1-c1ccc(N(c3ccc(-c4ccc(-c5ccccc5-c5ccccc5)cc4)cc3)c3ccc(C4CC5CCC4C5)cc3)cc1C2(c1ccccc1)c1ccccc1 ; c1ccc(-c2ccccc2-c2ccc(-c3ccc(N(c4ccc(C5CC6CCC5C6)cc4)c4ccc5c(c4)C(c4ccccc4)(c4ccccc4)c4cccc(-c6ccccc6)c4-5)cc3)cc2)cc1]

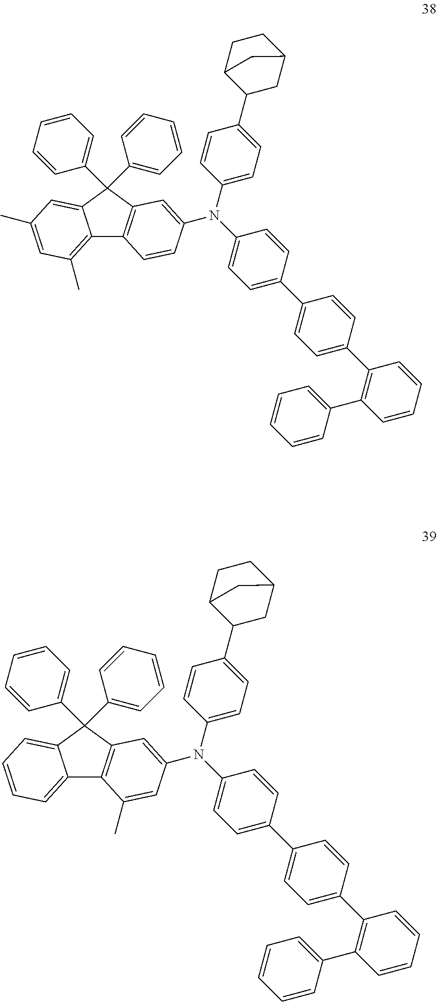 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C4CC5CCC4C5)cc3)ccc1-2 ; Cc1cc(N(c2ccc(-c3ccc(-c4ccccc4-c4ccccc4)cc3)cc2)c2ccc(C3CC4CCC3C4)cc2)cc2c1-c1ccccc1C2(c1ccccc1)c1ccccc1]

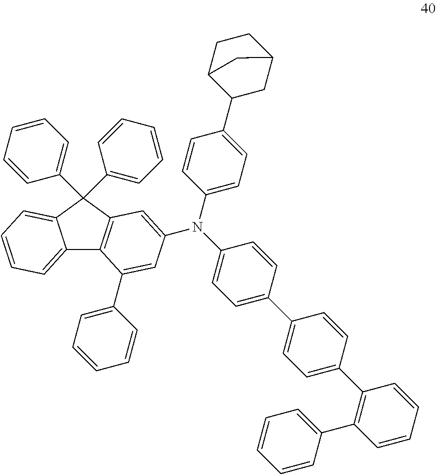 [SMILES: c1ccc(-c2ccccc2-c2ccc(-c3ccc(N(c4ccc(C5CC6CCC5C6)cc4)c4cc(-c5ccccc5)c5c(c4)C(c4ccccc4)(c4ccccc4)c4ccccc4-5)cc3)cc2)cc1]

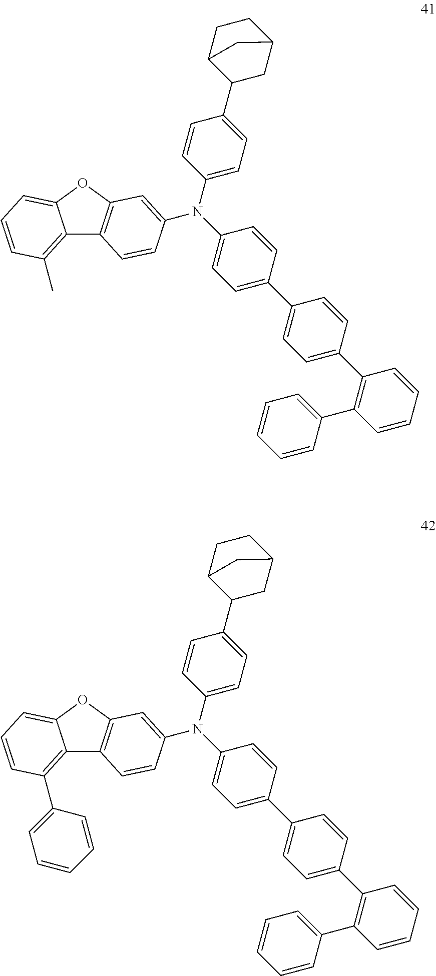 [SMILES: Cc1cccc2oc3cc(N(c4ccc(-c5ccc(-c6ccccc6-c6ccccc6)cc5)cc4)c4ccc(C5CC6CCC5C6)cc4)ccc3c12 ; c1ccc(-c2ccccc2-c2ccc(-c3ccc(N(c4ccc(C5CC6CCC5C6)cc4)c4ccc5c(c4)oc4cccc(-c6ccccc6)c45)cc3)cc2)cc1]

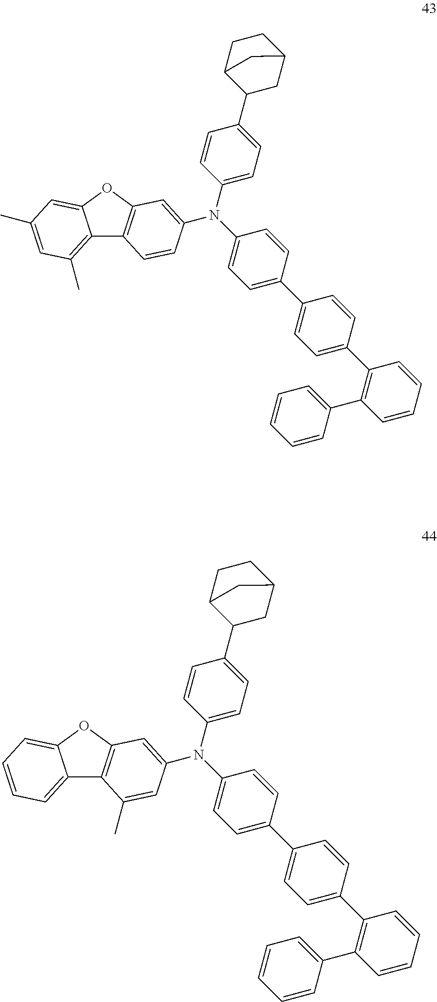 [SMILES: Cc1cc(C)c2c(c1)oc1cc(N(c3ccc(-c4ccc(-c5ccccc5-c5ccccc5)cc4)cc3)c3ccc(C4CC5CCC4C5)cc3)ccc12 ; Cc1cc(N(c2ccc(-c3ccc(-c4ccccc4-c4ccccc4)cc3)cc2)c2ccc(C3CC4CCC3C4)cc2)cc2oc3ccccc3c12]

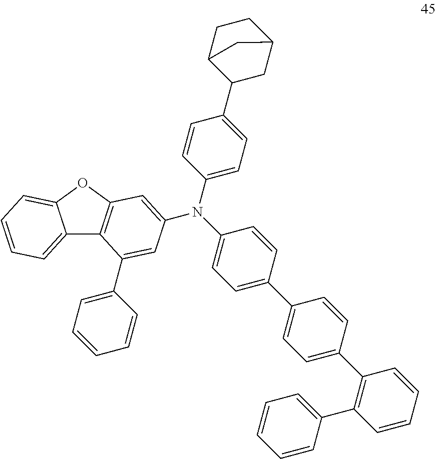 [SMILES: c1ccc(-c2ccccc2-c2ccc(-c3ccc(N(c4ccc(C5CC6CCC5C6)cc4)c4cc(-c5ccccc5)c5c(c4)oc4ccccc45)cc3)cc2)cc1]

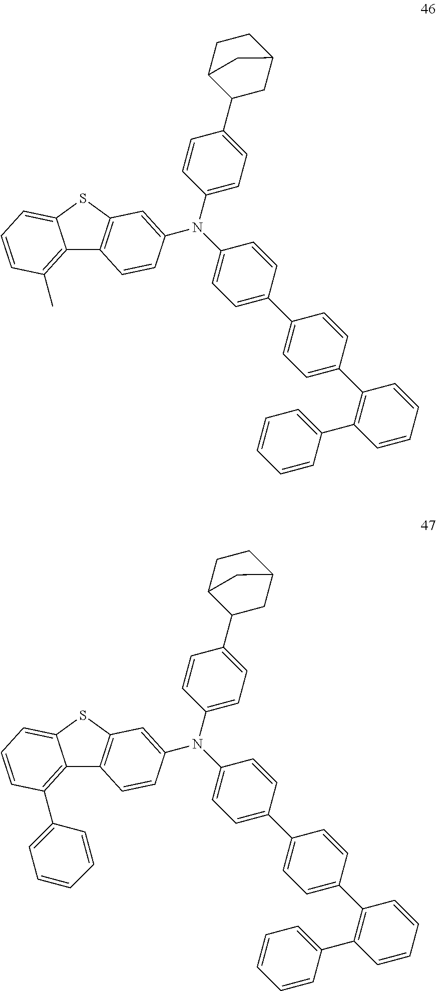 [SMILES: Cc1cccc2sc3cc(N(c4ccc(-c5ccc(-c6ccccc6-c6ccccc6)cc5)cc4)c4ccc(C5CC6CCC5C6)cc4)ccc3c12 ; c1ccc(-c2ccccc2-c2ccc(-c3ccc(N(c4ccc(C5CC6CCC5C6)cc4)c4ccc5c(c4)sc4cccc(-c6ccccc6)c45)cc3)cc2)cc1]

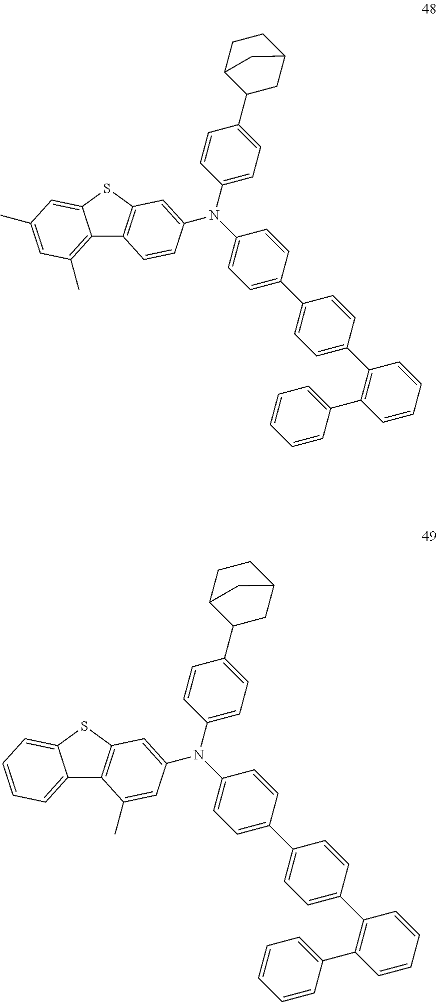 [SMILES: Cc1cc(C)c2c(c1)sc1cc(N(c3ccc(-c4ccc(-c5ccccc5-c5ccccc5)cc4)cc3)c3ccc(C4CC5CCC4C5)cc3)ccc12 ; Cc1cc(N(c2ccc(-c3ccc(-c4ccccc4-c4ccccc4)cc3)cc2)c2ccc(C3CC4CCC3C4)cc2)cc2sc3ccccc3c12]

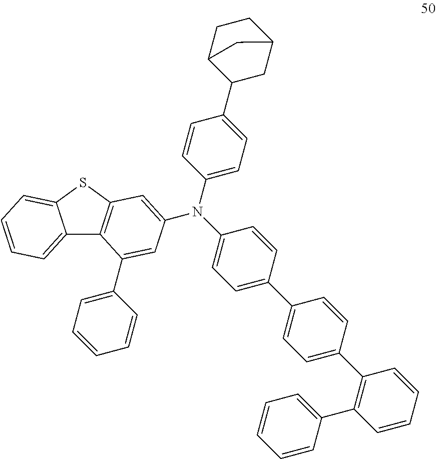 [SMILES: c1ccc(-c2ccccc2-c2ccc(-c3ccc(N(c4ccc(C5CC6CCC5C6)cc4)c4cc(-c5ccccc5)c5c(c4)sc4ccccc45)cc3)cc2)cc1]

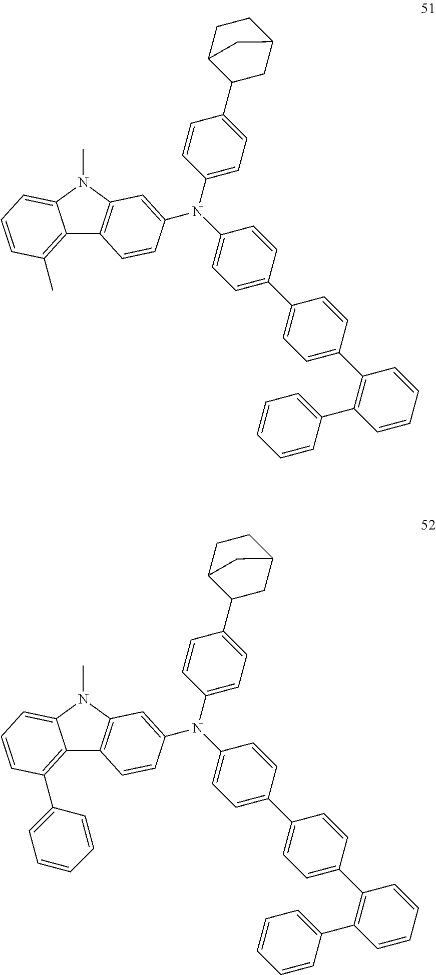 [SMILES: Cc1cccc2c1c1ccc(N(c3ccc(-c4ccc(-c5ccccc5-c5ccccc5)cc4)cc3)c3ccc(C4CC5CCC4C5)cc3)cc1n2C ; Cn1c2cc(N(c3ccc(-c4ccc(-c5ccccc5-c5ccccc5)cc4)cc3)c3ccc(C4CC5CCC4C5)cc3)ccc2c2c(-c3ccccc3)cccc21]

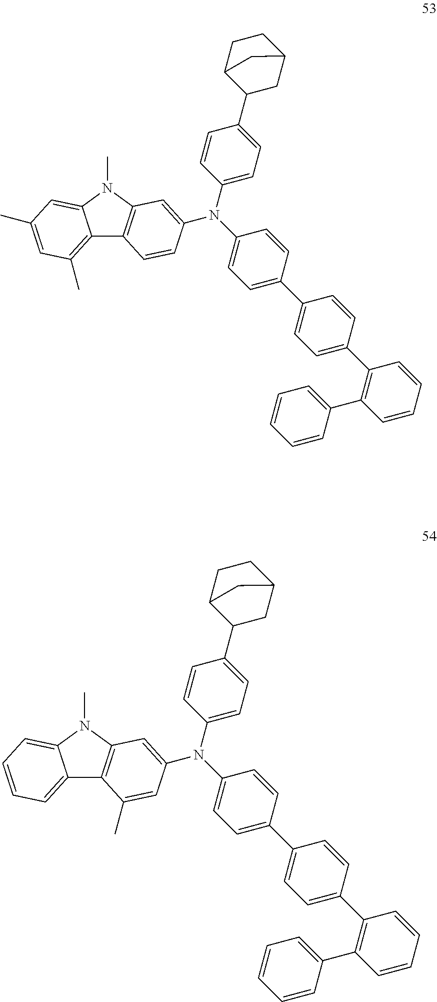 [SMILES: Cc1cc(C)c2c3ccc(N(c4ccc(-c5ccc(-c6ccccc6-c6ccccc6)cc5)cc4)c4ccc(C5CC6CCC5C6)cc4)cc3n(C)c2c1 ; Cc1cc(N(c2ccc(-c3ccc(-c4ccccc4-c4ccccc4)cc3)cc2)c2ccc(C3CC4CCC3C4)cc2)cc2c1c1ccccc1n2C]

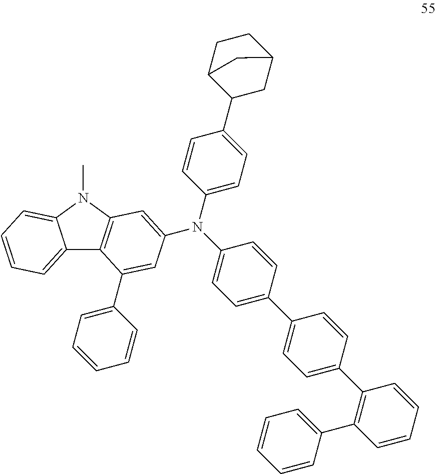 [SMILES: Cn1c2ccccc2c2c(-c3ccccc3)cc(N(c3ccc(-c4ccc(-c5ccccc5-c5ccccc5)cc4)cc3)c3ccc(C4CC5CCC4C5)cc3)cc21]

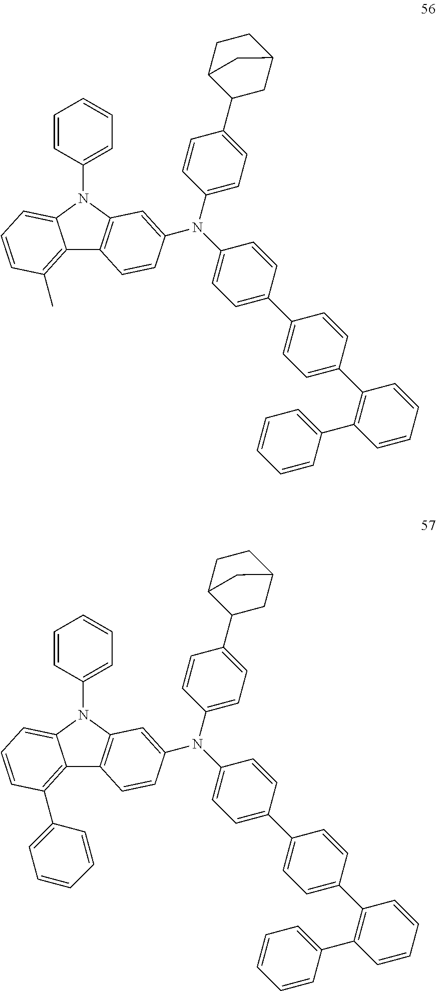 [SMILES: Cc1cccc2c1c1ccc(N(c3ccc(-c4ccc(-c5ccccc5-c5ccccc5)cc4)cc3)c3ccc(C4CC5CCC4C5)cc3)cc1n2-c1ccccc1 ; c1ccc(-c2ccccc2-c2ccc(-c3ccc(N(c4ccc(C5CC6CCC5C6)cc4)c4ccc5c6c(-c7ccccc7)cccc6n(-c6ccccc6)c5c4)cc3)cc2)cc1]

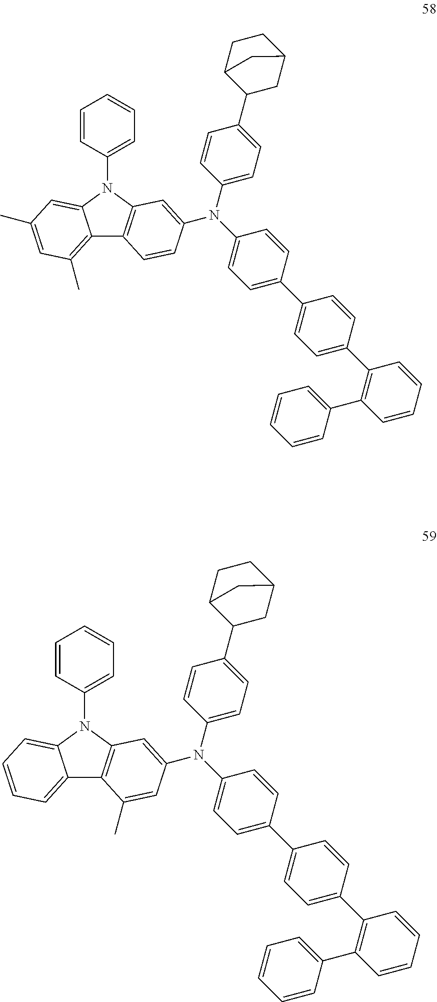 [SMILES: Cc1cc(C)c2c3ccc(N(c4ccc(-c5ccc(-c6ccccc6-c6ccccc6)cc5)cc4)c4ccc(C5CC6CCC5C6)cc4)cc3n(-c3ccccc3)c2c1 ; Cc1cc(N(c2ccc(-c3ccc(-c4ccccc4-c4ccccc4)cc3)cc2)c2ccc(C3CC4CCC3C4)cc2)cc2c1c1ccccc1n2-c1ccccc1]

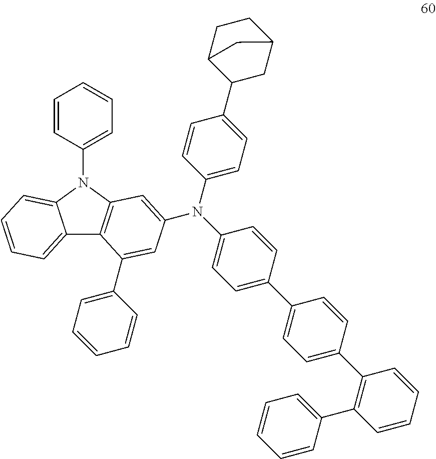 [SMILES: c1ccc(-c2ccccc2-c2ccc(-c3ccc(N(c4ccc(C5CC6CCC5C6)cc4)c4cc(-c5ccccc5)c5c6ccccc6n(-c6ccccc6)c5c4)cc3)cc2)cc1]

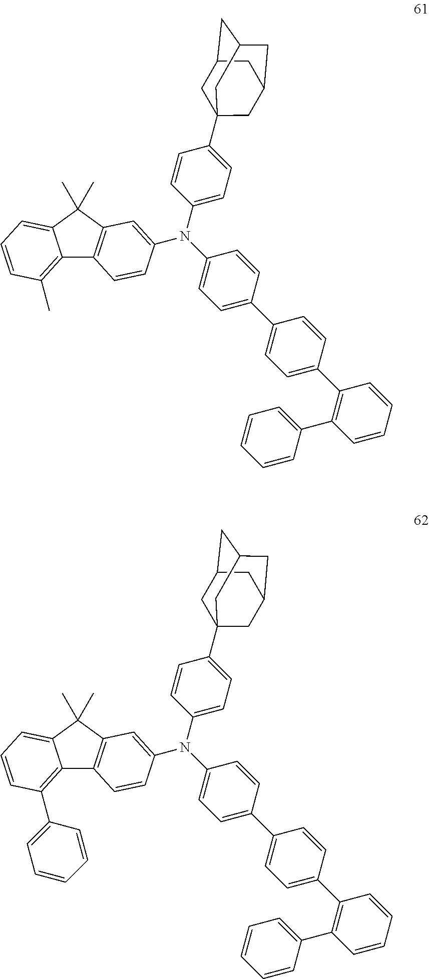 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(C)C ; CC1(C)c2cc(N(c3ccc(-c4ccc(-c5ccccc5-c5ccccc5)cc4)cc3)c3ccc(C45CC6CC(CC(C6)C4)C5)cc3)ccc2-c2c(-c3ccccc3)cccc21]

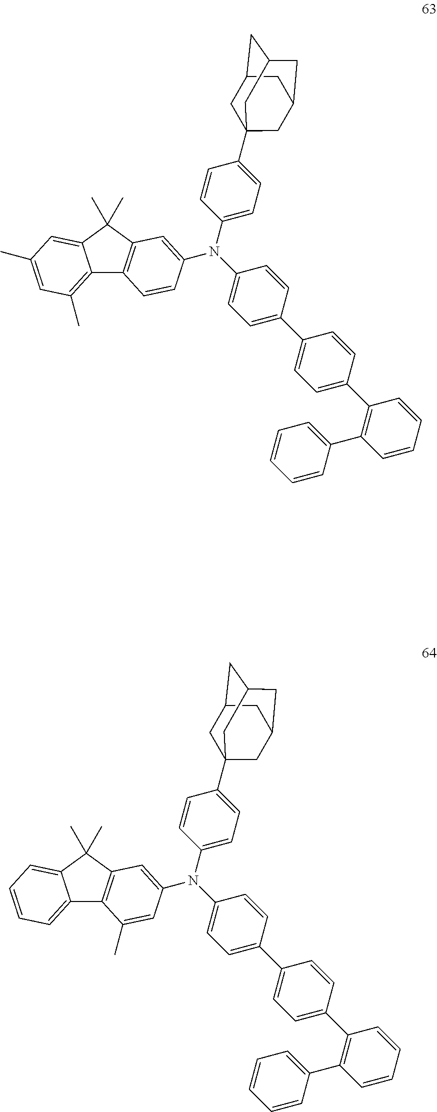 [SMILES: Cc1cc(C)c2c(c1)C(C)(C)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(C)C]

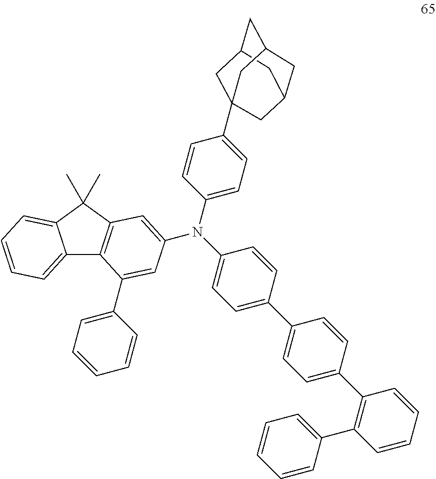 [SMILES: CC1(C)c2ccccc2-c2c(-c3ccccc3)cc(N(c3ccc(-c4ccc(-c5ccccc5-c5ccccc5)cc4)cc3)c3ccc(C45CC6CC(CC(C6)C4)C5)cc3)cc21]

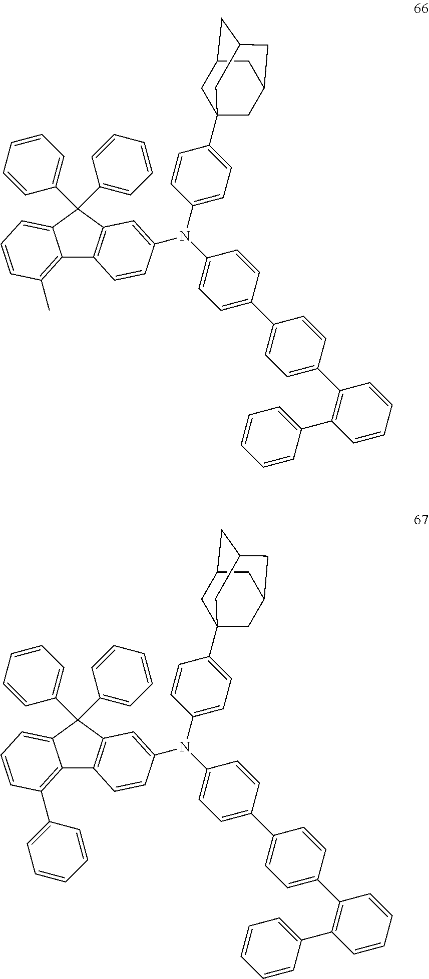 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(c1ccccc1)c1ccccc1 ; c1ccc(-c2ccccc2-c2ccc(-c3ccc(N(c4ccc(C56CC7CC(CC(C7)C5)C6)cc4)c4ccc5c(c4)C(c4ccccc4)(c4ccccc4)c4cccc(-c6ccccc6)c4-5)cc3)cc2)cc1]

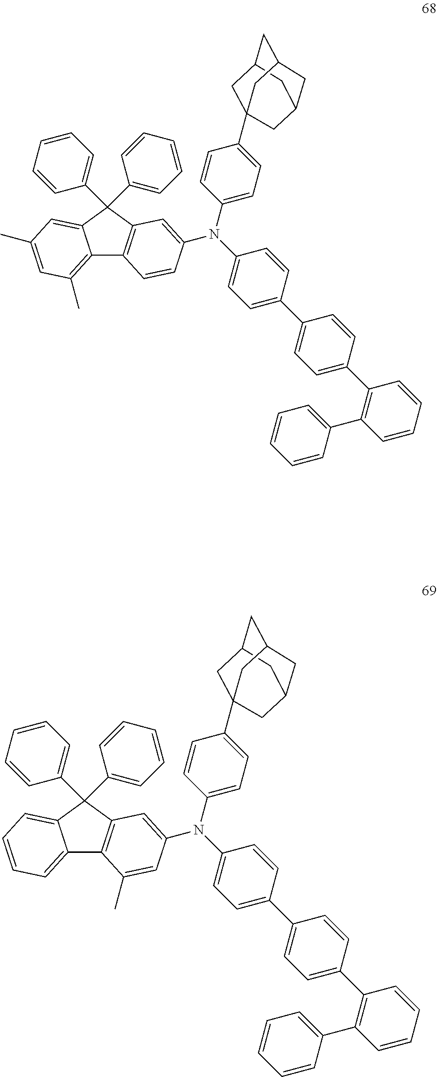 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(c1ccccc1)c1ccccc1]

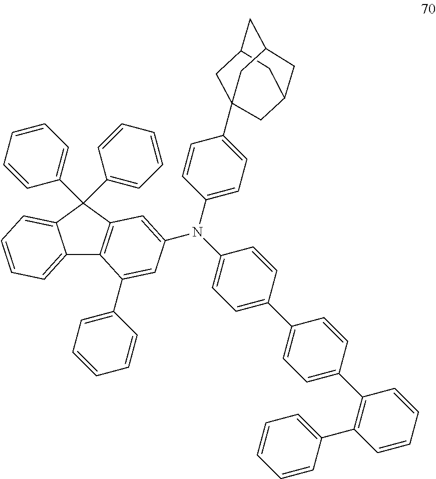 [SMILES: c1ccc(-c2ccccc2-c2ccc(-c3ccc(N(c4ccc(C56CC7CC(CC(C7)C5)C6)cc4)c4cc(-c5ccccc5)c5c(c4)C(c4ccccc4)(c4ccccc4)c4ccccc4-5)cc3)cc2)cc1]

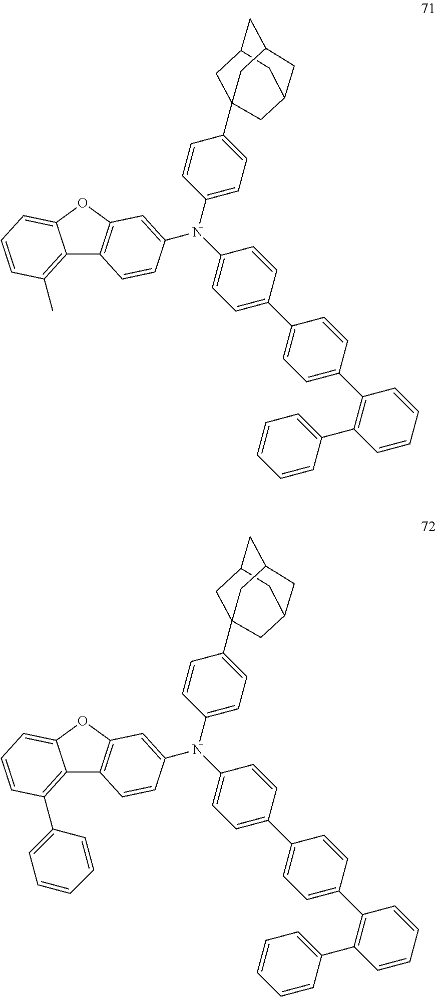 [SMILES: Cc1cccc2oc3cc(N(c4ccc(-c5ccc(-c6ccccc6-c6ccccc6)cc5)cc4)c4ccc(C56CC7CC(CC(C7)C5)C6)cc4)ccc3c12 ; c1ccc(-c2ccccc2-c2ccc(-c3ccc(N(c4ccc(C56CC7CC(CC(C7)C5)C6)cc4)c4ccc5c(c4)oc4cccc(-c6ccccc6)c45)cc3)cc2)cc1]

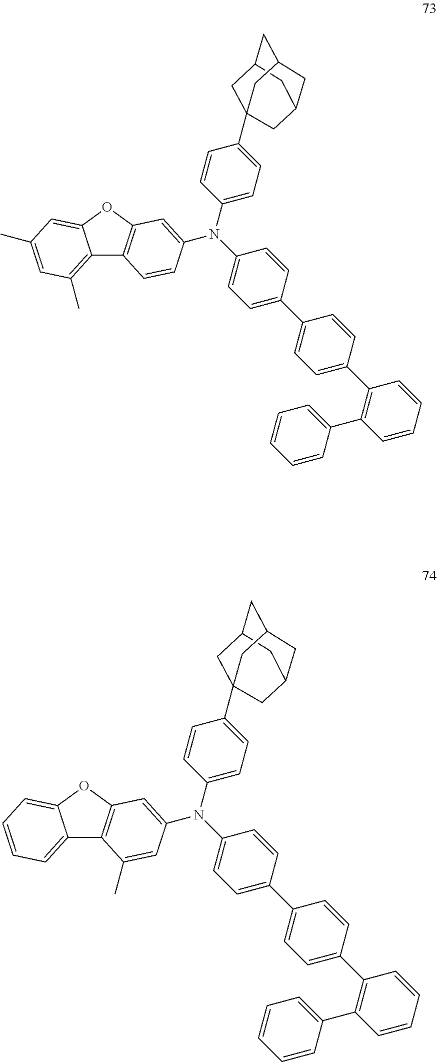 [SMILES: Cc1cc(C)c2c(c1)oc1cc(N(c3ccc(-c4ccc(-c5ccccc5-c5ccccc5)cc4)cc3)c3ccc(C45CC6CC(CC(C6)C4)C5)cc3)ccc12 ; Cc1cc(N(c2ccc(-c3ccc(-c4ccccc4-c4ccccc4)cc3)cc2)c2ccc(C34CC5CC(CC(C5)C3)C4)cc2)cc2oc3ccccc3c12]

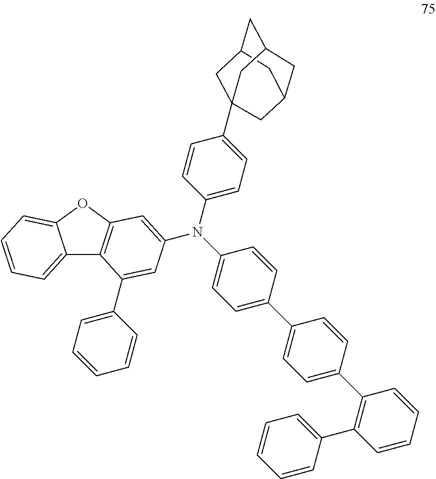 [SMILES: c1ccc(-c2ccccc2-c2ccc(-c3ccc(N(c4ccc(C56CC7CC(CC(C7)C5)C6)cc4)c4cc(-c5ccccc5)c5c(c4)oc4ccccc45)cc3)cc2)cc1]

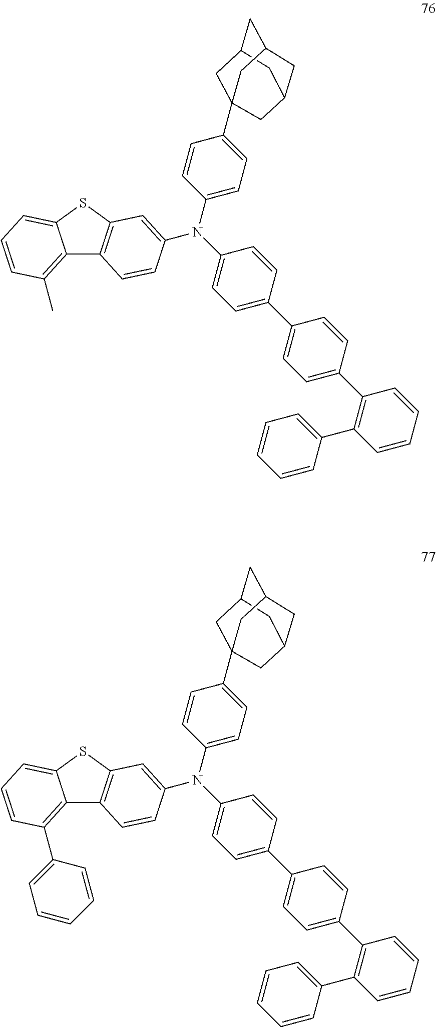 [SMILES: Cc1cccc2sc3cc(N(c4ccc(-c5ccc(-c6ccccc6-c6ccccc6)cc5)cc4)c4ccc(C56CC7CC(CC(C7)C5)C6)cc4)ccc3c12 ; c1ccc(-c2ccccc2-c2ccc(-c3ccc(N(c4ccc(C56CC7CC(CC(C7)C5)C6)cc4)c4ccc5c(c4)sc4cccc(-c6ccccc6)c45)cc3)cc2)cc1]

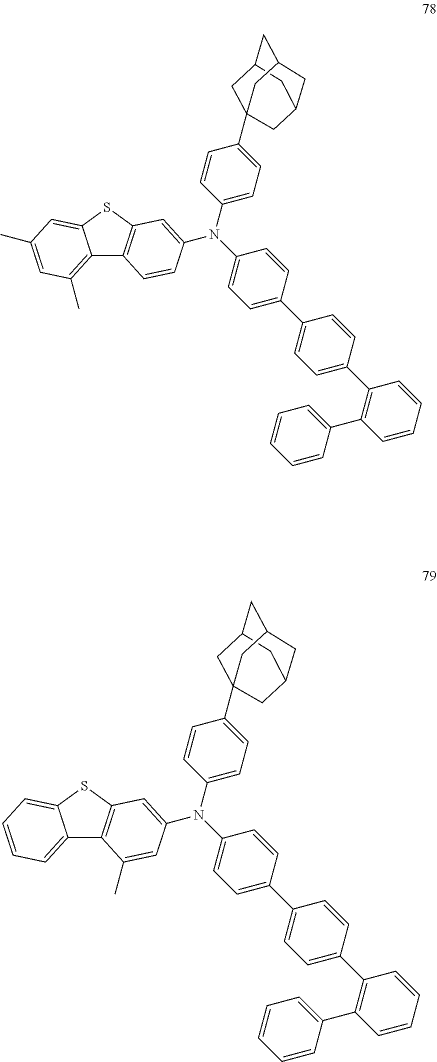 [SMILES: Cc1cc(C)c2c(c1)sc1cc(N(c3ccc(-c4ccc(-c5ccccc5-c5ccccc5)cc4)cc3)c3ccc(C45CC6CC(CC(C6)C4)C5)cc3)ccc12 ; Cc1cc(N(c2ccc(-c3ccc(-c4ccccc4-c4ccccc4)cc3)cc2)c2ccc(C34CC5CC(CC(C5)C3)C4)cc2)cc2sc3ccccc3c12]

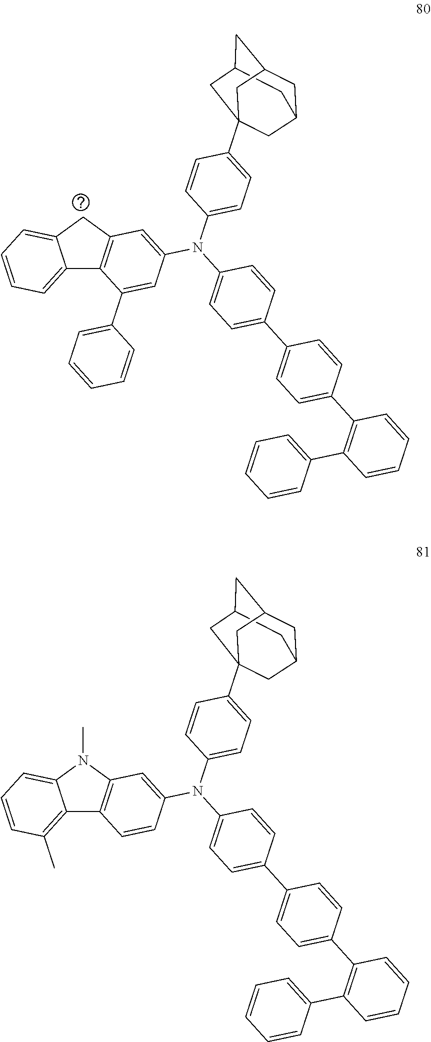 [SMILES: c1ccc(-c2ccccc2-c2ccc(-c3ccc(N(c4ccc(C56CC7CC(CC(C7)C5)C6)cc4)c4cc5c(c(-c6ccccc6)c4)-c4ccccc4C5)cc3)cc2)cc1 ; Cc1cccc2c1c1ccc(N(c3ccc(-c4ccc(-c5ccccc5-c5ccccc5)cc4)cc3)c3ccc(C45CC6CC(CC(C6)C4)C5)cc3)cc1n2C]

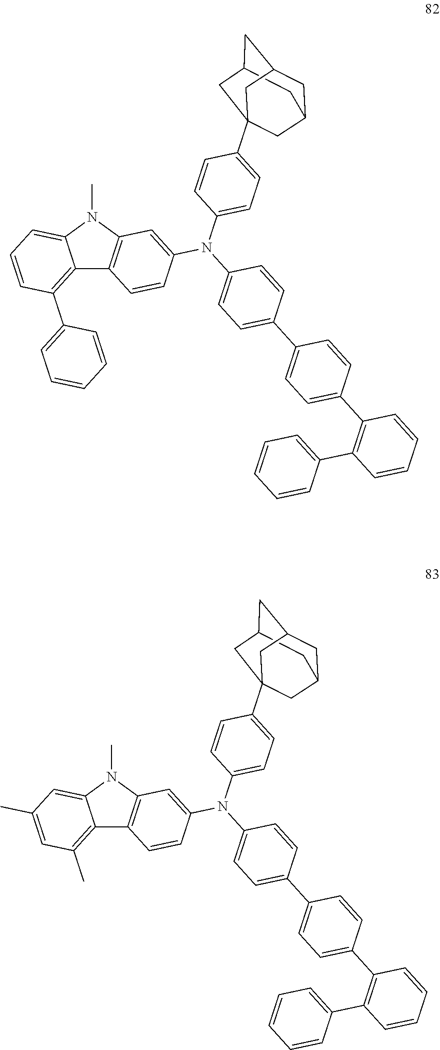 [SMILES: Cn1c2cc(N(c3ccc(-c4ccc(-c5ccccc5-c5ccccc5)cc4)cc3)c3ccc(C45CC6CC(CC(C6)C4)C5)cc3)ccc2c2c(-c3ccccc3)cccc21 ; Cc1cc(C)c2c3ccc(N(c4ccc(-c5ccc(-c6ccccc6-c6ccccc6)cc5)cc4)c4ccc(C56CC7CC(CC(C7)C5)C6)cc4)cc3n(C)c2c1]

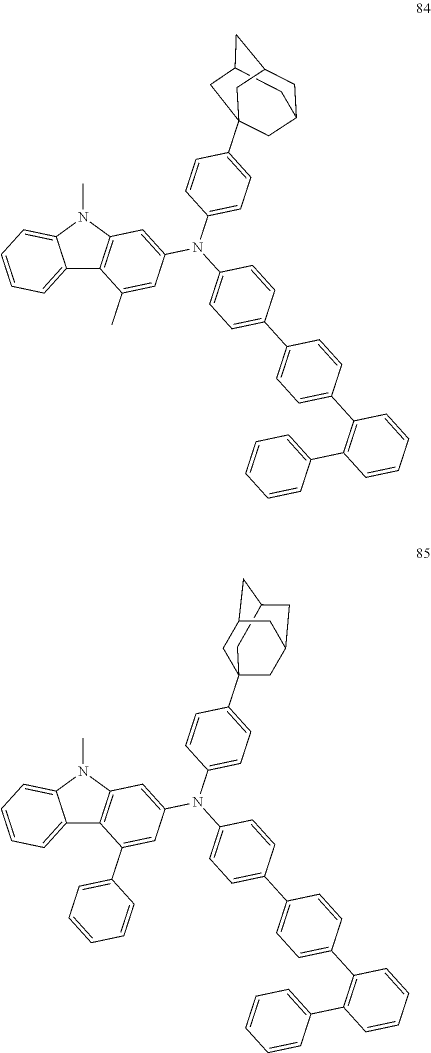 [SMILES: Cc1cc(N(c2ccc(-c3ccc(-c4ccccc4-c4ccccc4)cc3)cc2)c2ccc(C34CC5CC(CC(C5)C3)C4)cc2)cc2c1c1ccccc1n2C ; Cn1c2ccccc2c2c(-c3ccccc3)cc(N(c3ccc(-c4ccc(-c5ccccc5-c5ccccc5)cc4)cc3)c3ccc(C45CC6CC(CC(C6)C4)C5)cc3)cc21]

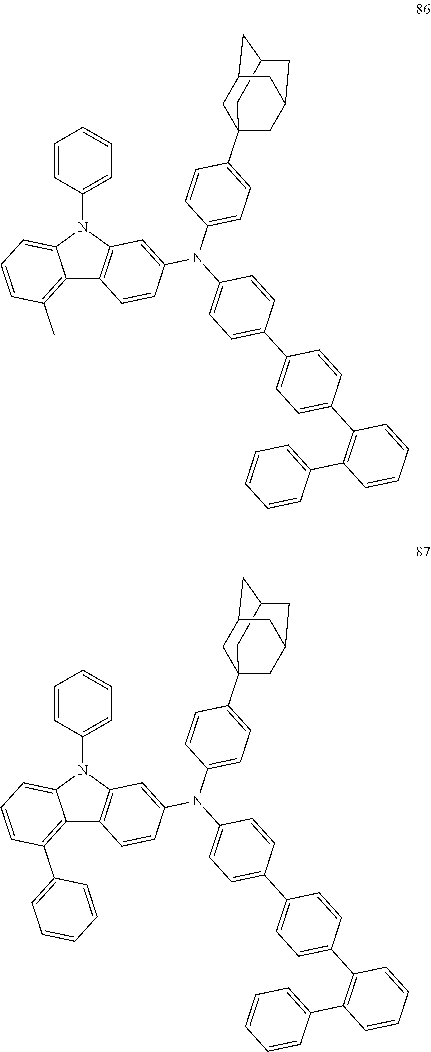 [SMILES: Cc1cccc2c1c1ccc(N(c3ccc(-c4ccc(-c5ccccc5-c5ccccc5)cc4)cc3)c3ccc(C45CC6CC(CC(C6)C4)C5)cc3)cc1n2-c1ccccc1 ; c1ccc(-c2ccccc2-c2ccc(-c3ccc(N(c4ccc(C56CC7CC(CC(C7)C5)C6)cc4)c4ccc5c6c(-c7ccccc7)cccc6n(-c6ccccc6)c5c4)cc3)cc2)cc1]

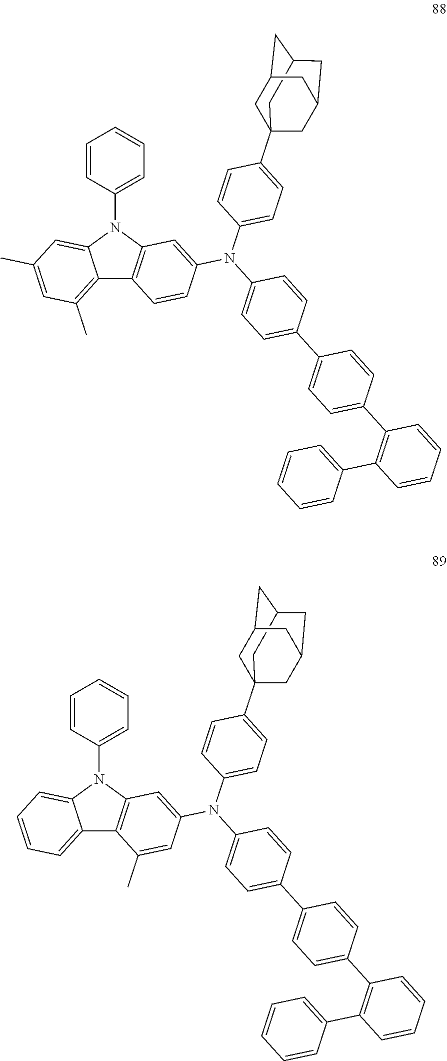 [SMILES: Cc1cc(C)c2c3ccc(N(c4ccc(-c5ccc(-c6ccccc6-c6ccccc6)cc5)cc4)c4ccc(C56CC7CC(CC(C7)C5)C6)cc4)cc3n(-c3ccccc3)c2c1 ; Cc1cc(N(c2ccc(-c3ccc(-c4ccccc4-c4ccccc4)cc3)cc2)c2ccc(C34CC5CC(CC(C5)C3)C4)cc2)cc2c1c1ccccc1n2-c1ccccc1]

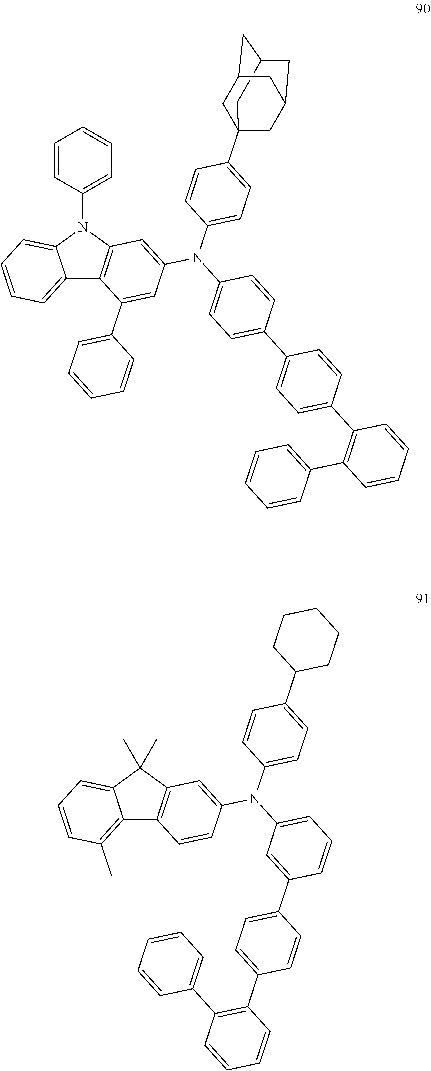 [SMILES: c1ccc(-c2ccccc2-c2ccc(-c3ccc(N(c4ccc(C56CC7CC(CC(C7)C5)C6)cc4)c4cc(-c5ccccc5)c5c6ccccc6n(-c6ccccc6)c5c4)cc3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2(C)C]

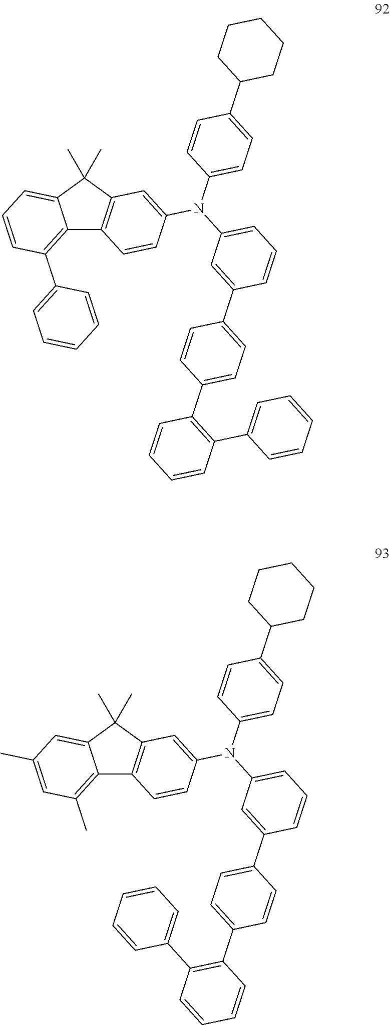 [SMILES: CC1(C)c2cc(N(c3ccc(C4CCCCC4)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C4CCCCC4)cc3)c3cccc(-c4ccc(-c5ccccc5-c5ccccc5)cc4)c3)ccc1-2]

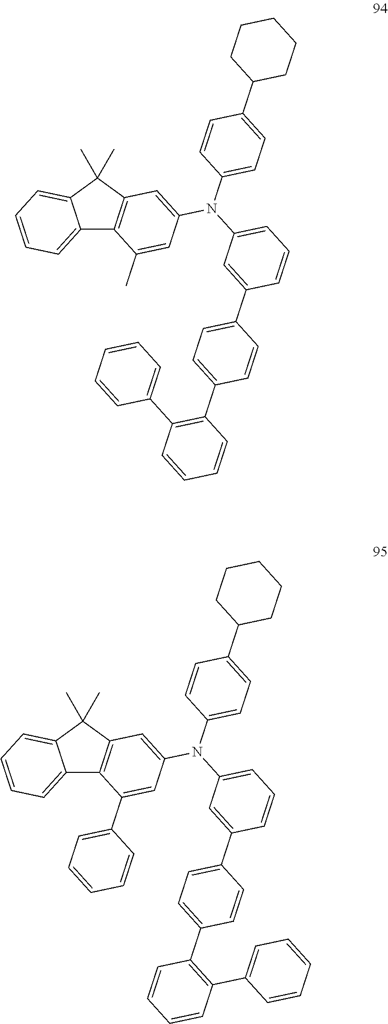 [SMILES: Cc1cc(N(c2ccc(C3CCCCC3)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CCCCC4)cc3)c3cccc(-c4ccc(-c5ccccc5-c5ccccc5)cc4)c3)cc21]

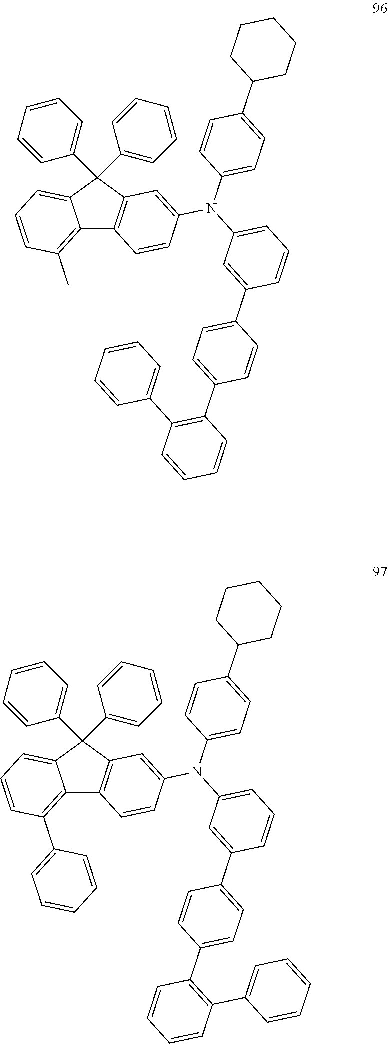 [SMILES: Cc1cccc2c1-c1ccc(N(c3ccc(C4CCCCC4)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CCCCC5)cc4)c4ccc5c(c4)C(c4ccccc4)(c4ccccc4)c4cccc(-c6ccccc6)c4-5)c3)cc2)cc1]

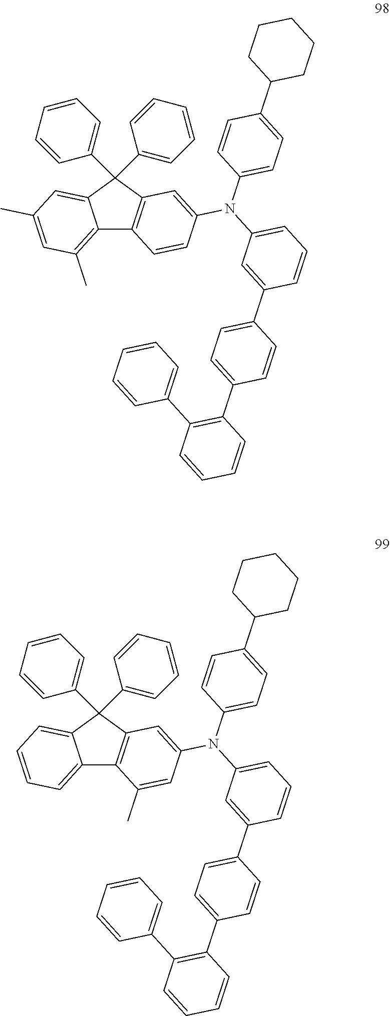 [SMILES: Cc1cc(C)c2c(c1)C(c1ccccc1)(c1ccccc1)c1cc(N(c3ccc(C4CCCCC4)cc3)c3cccc(-c4ccc(-c5ccccc5-c5ccccc5)cc4)c3)ccc1-2 ; Cc1cc(N(c2ccc(C3CCCCC3)cc2)c2cccc(-c3ccc(-c4ccccc4-c4ccccc4)cc3)c2)cc2c1-c1ccccc1C2(c1ccccc1)c1ccccc1]

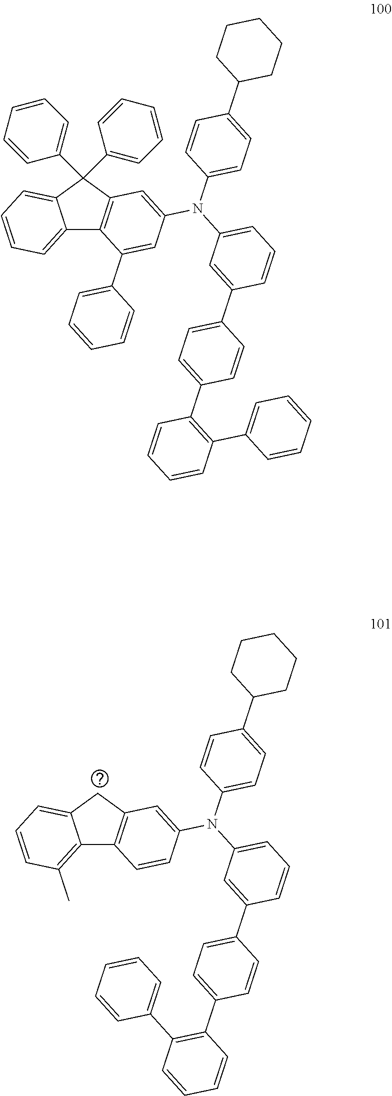 [SMILES: c1ccc(-c2ccccc2-c2ccc(-c3cccc(N(c4ccc(C5CCCCC5)cc4)c4cc(-c5ccccc5)c5c(c4)C(c4ccccc4)(c4ccccc4)c4ccccc4-5)c3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2]

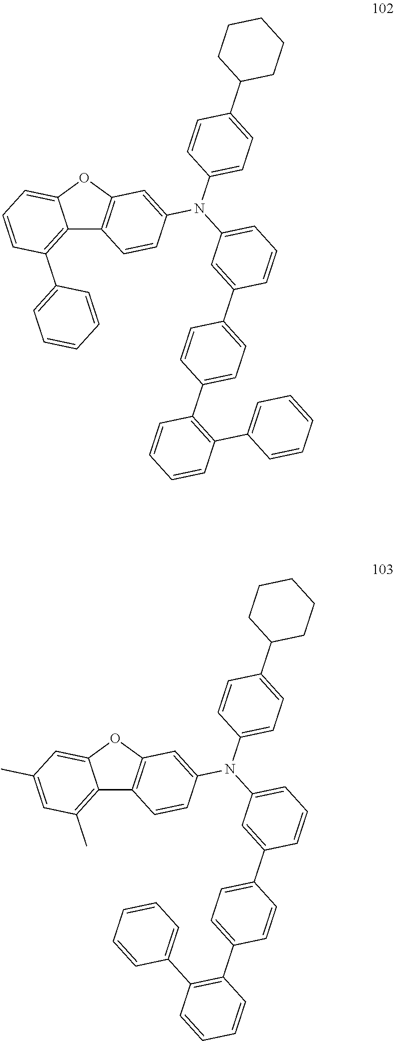 [SMILES: c1ccc(-c2ccccc2-c2ccc(-c3cccc(N(c4ccc(C5CCCCC5)cc4)c4ccc5c(c4)oc4cccc(-c6ccccc6)c45)c3)cc2)cc1 ; Cc1cc(C)c2c(c1)oc1cc(N(c3ccc(C4CCCCC4)cc3)c3cccc(-c4ccc(-c5ccccc5-c5ccccc5)cc4)c3)ccc12]

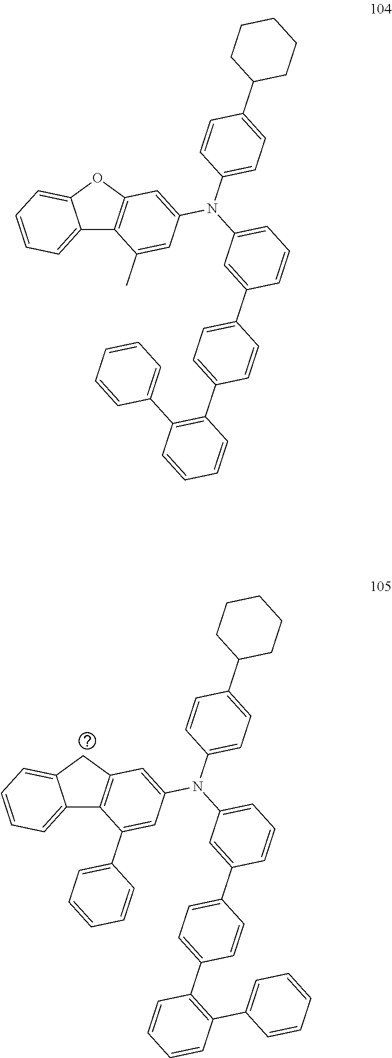 [SMILES: Cc1cc(N(c2ccc(C3CCCCC3)cc2)c2cccc(-c3ccc(-c4ccccc4-c4ccccc4)cc3)c2)cc2oc3ccccc3c12 ; c1ccc(-c2ccccc2-c2ccc(-c3cccc(N(c4ccc(C5CCCCC5)cc4)c4cc5c(c(-c6ccccc6)c4)-c4ccccc4C5)c3)cc2)cc1]

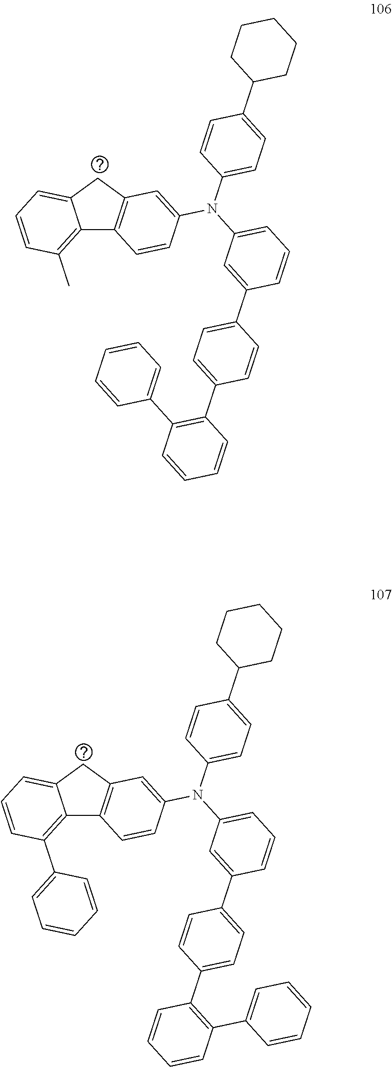 [SMILES: Cc1cccc2c1-c1ccc(N(c3ccc(C4CCCCC4)cc3)c3cccc(-c4ccc(-c5ccccc5-c5ccccc5)cc4)c3)cc1C2 ; c1ccc(-c2ccccc2-c2ccc(-c3cccc(N(c4ccc(C5CCCCC5)cc4)c4ccc5c(c4)Cc4cccc(-c6ccccc6)c4-5)c3)cc2)cc1]

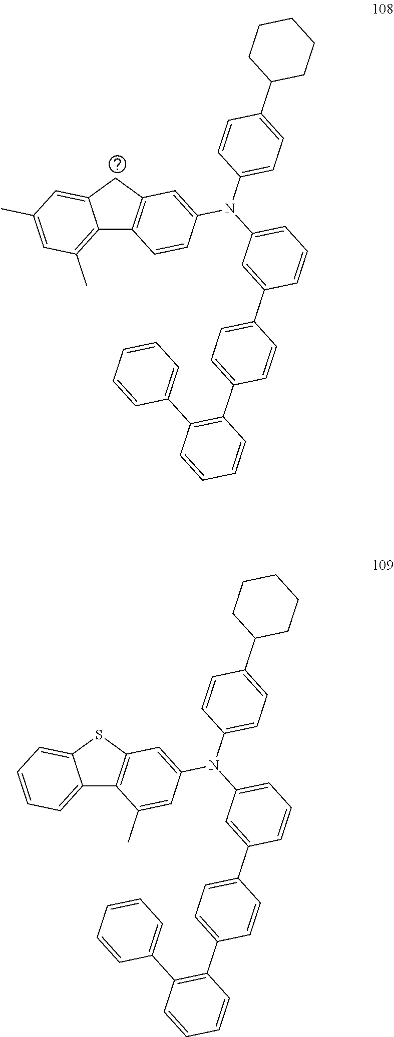 [SMILES: Cc1cc(C)c2c(c1)Cc1cc(N(c3ccc(C4CCCCC4)cc3)c3cccc(-c4ccc(-c5ccccc5-c5ccccc5)cc4)c3)ccc1-2 ; Cc1cc(N(c2ccc(C3CCCCC3)cc2)c2cccc(-c3ccc(-c4ccccc4-c4ccccc4)cc3)c2)cc2sc3ccccc3c12]

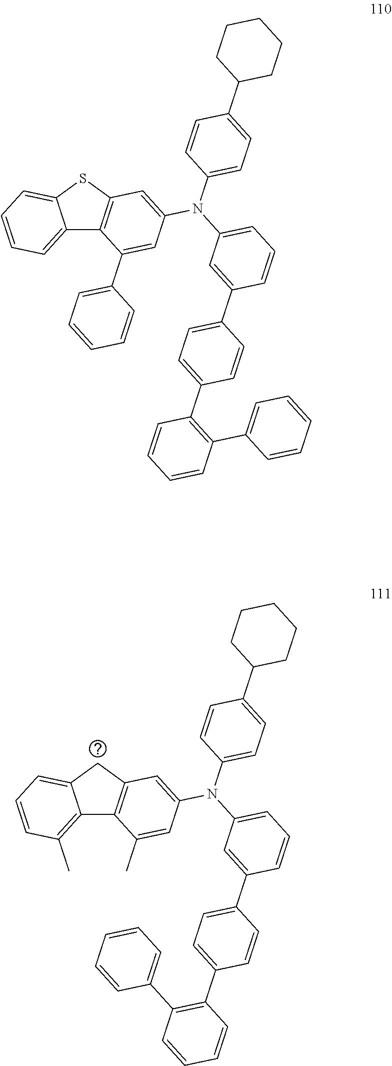 [SMILES: c1ccc(-c2ccccc2-c2ccc(-c3cccc(N(c4ccc(C5CCCCC5)cc4)c4cc(-c5ccccc5)c5c(c4)sc4ccccc45)c3)cc2)cc1 ; Cc1cccc2c1-c1c(C)cc(N(c3ccc(C4CCCCC4)cc3)c3cccc(-c4ccc(-c5ccccc5-c5ccccc5)cc4)c3)cc1C2]

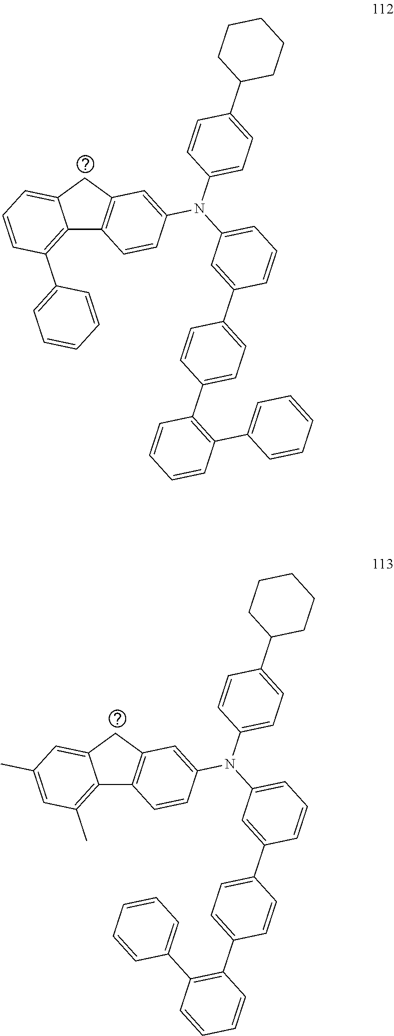 [SMILES: c1ccc(-c2ccccc2-c2ccc(-c3cccc(N(c4ccc(C5CCCCC5)cc4)c4ccc5c(c4)Cc4cccc(-c6ccccc6)c4-5)c3)cc2)cc1 ; Cc1cc(C)c2c(c1)Cc1cc(N(c3ccc(C4CCCCC4)cc3)c3cccc(-c4ccc(-c5ccccc5-c5ccccc5)cc4)c3)ccc1-2]

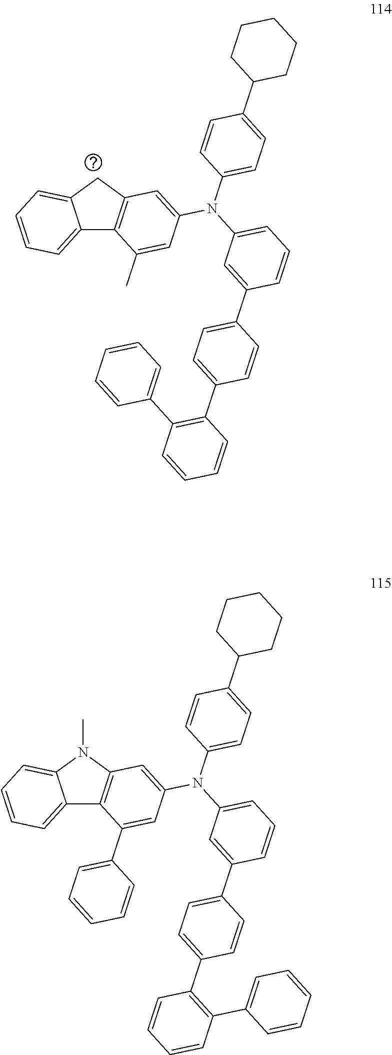 [SMILES: Cc1cc(N(c2ccc(C3CCCCC3)cc2)c2cccc(-c3ccc(-c4ccccc4-c4ccccc4)cc3)c2)cc2c1-c1ccccc1C2 ; Cn1c2ccccc2c2c(-c3ccccc3)cc(N(c3ccc(C4CCCCC4)cc3)c3cccc(-c4ccc(-c5ccccc5-c5ccccc5)cc4)c3)cc21]

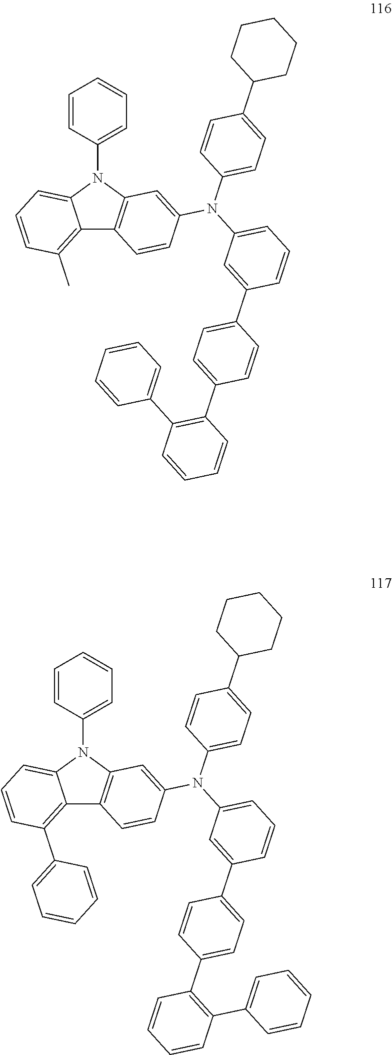 [SMILES: Cc1cccc2c1c1ccc(N(c3ccc(C4CCCCC4)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C5CCCCC5)cc4)c4ccc5c6c(-c7ccccc7)cccc6n(-c6ccccc6)c5c4)c3)cc2)cc1]

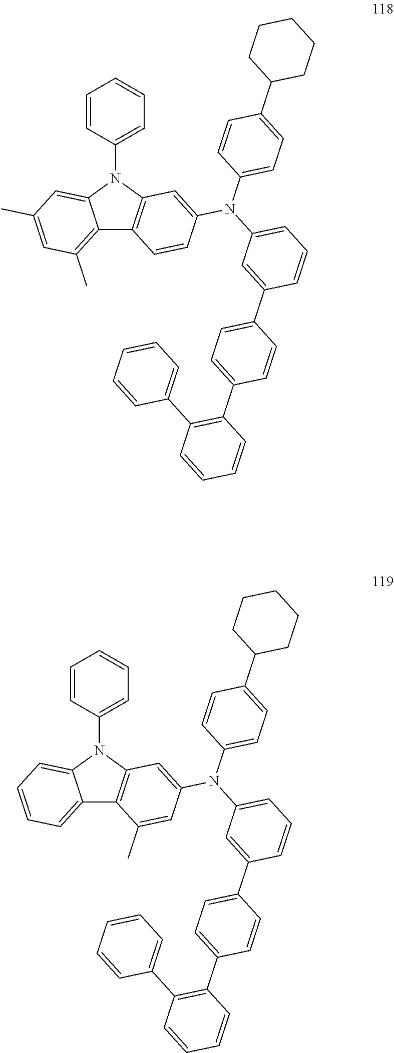 [SMILES: Cc1cc(C)c2c3ccc(N(c4ccc(C5CCCCC5)cc4)c4cccc(-c5ccc(-c6ccccc6-c6ccccc6)cc5)c4)cc3n(-c3ccccc3)c2c1 ; Cc1cc(N(c2ccc(C3CCCCC3)cc2)c2cccc(-c3ccc(-c4ccccc4-c4ccccc4)cc3)c2)cc2c1c1ccccc1n2-c1ccccc1]

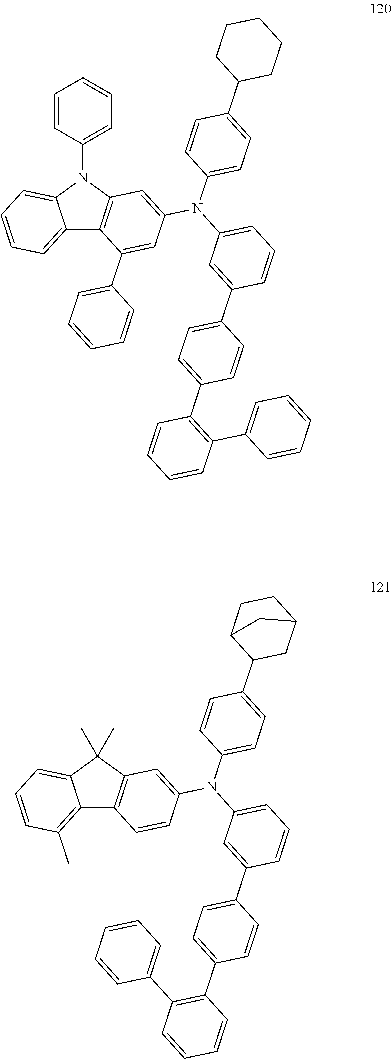 [SMILES: c1ccc(-c2ccccc2-c2ccc(-c3cccc(N(c4ccc(C5CCCCC5)cc4)c4cc(-c5ccccc5)c5c6ccccc6n(-c6ccccc6)c5c4)c3)cc2)cc1 ; Cc1cccc2c1-c1ccc(N(c3ccc(C4CC5CCC4C5)cc3)c3cccc(-c4ccc(-c5ccccc5-c5ccccc5)cc4)c3)cc1C2(C)C]

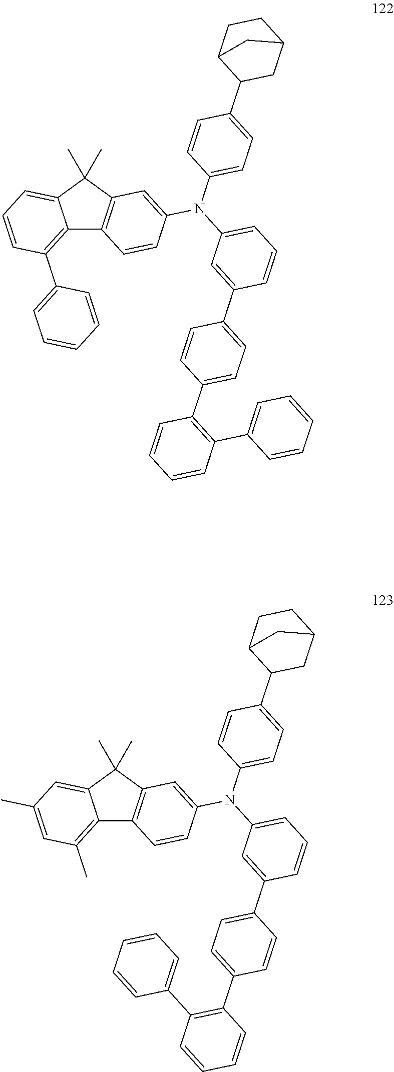 [SMILES: CC1(C)c2cc(N(c3ccc(C4CC5CCC4C5)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C4CC5CCC4C5)cc3)c3cccc(-c4ccc(-c5ccccc5-c5ccccc5)cc4)c3)ccc1-2]

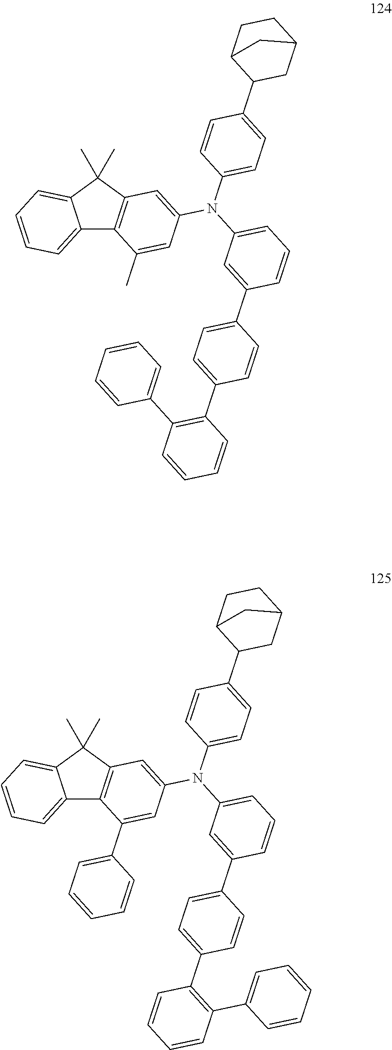 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CC5CCC4C5)cc3)c3cccc(-c4ccc(-c5ccccc5-c5ccccc5)cc4)c3)cc21]

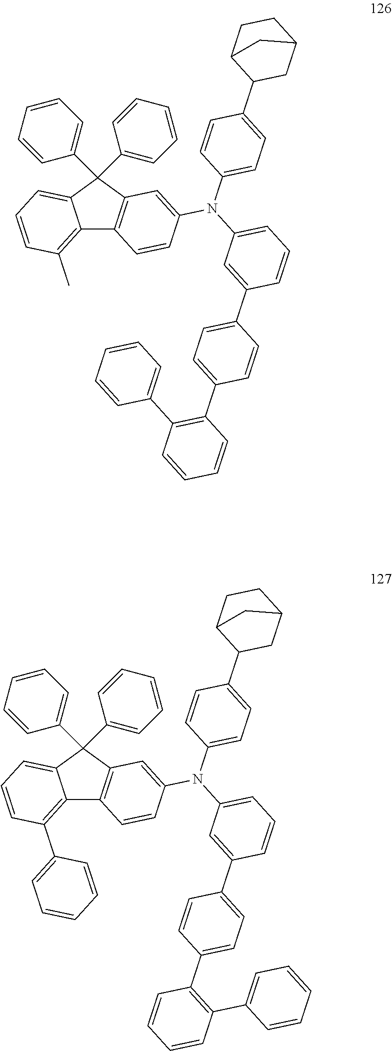 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CC6CCC5C6)cc4)c4ccc5c(c4)C(c4ccccc4)(c4ccccc4)c4cccc(-c6ccccc6)c4-5)c3)cc2)cc1]

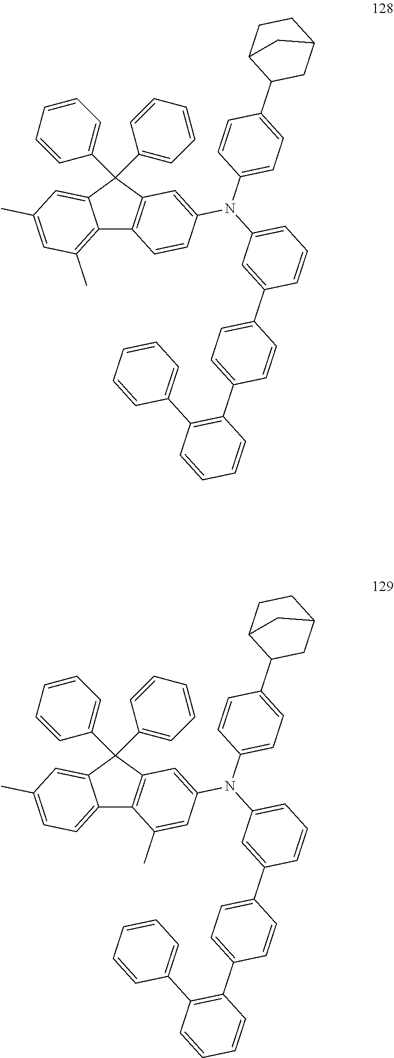 [SMILES: Cc1cc(C)c2c(c1)C(c1ccccc1)(c1ccccc1)c1cc(N(c3ccc(C4CC5CCC4C5)cc3)c3cccc(-c4ccc(-c5ccccc5-c5ccccc5)cc4)c3)ccc1-2 ; Cc1ccc2c(c1)C(c1ccccc1)(c1ccccc1)c1cc(N(c3ccc(C4CC5CCC4C5)cc3)c3cccc(-c4ccc(-c5ccccc5-c5ccccc5)cc4)c3)cc(C)c1-2]

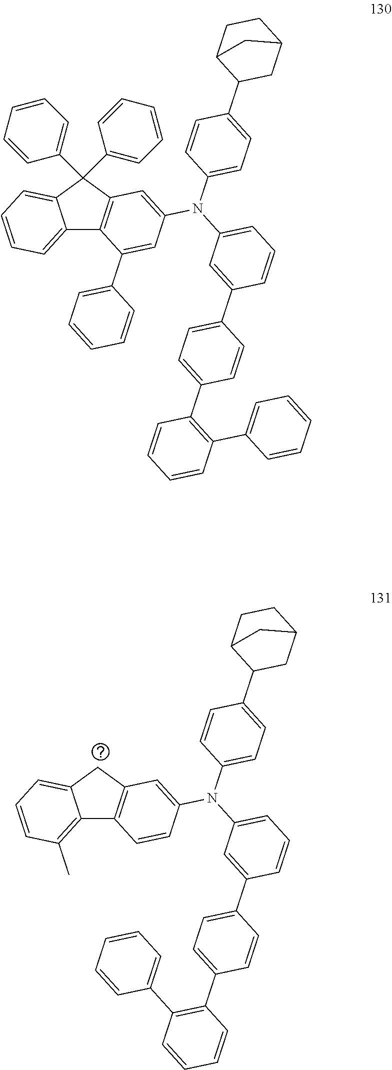 [SMILES: c1ccc(-c2ccccc2-c2ccc(-c3cccc(N(c4ccc(C5CC6CCC5C6)cc4)c4cc(-c5ccccc5)c5c(c4)C(c4ccccc4)(c4ccccc4)c4ccccc4-5)c3)cc2)cc1 ; Cc1cccc2c1-c1ccc(N(c3ccc(C4CC5CCC4C5)cc3)c3cccc(-c4ccc(-c5ccccc5-c5ccccc5)cc4)c3)cc1C2]

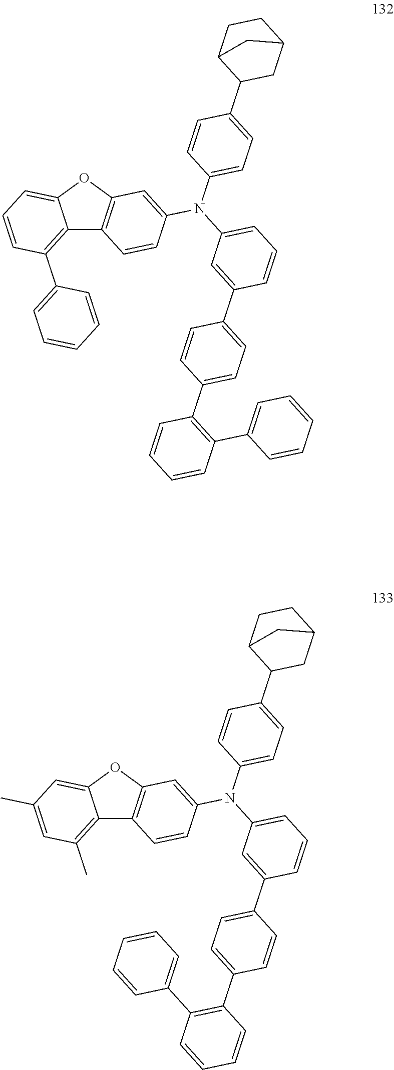 [SMILES: c1ccc(-c2ccccc2-c2ccc(-c3cccc(N(c4ccc(C5CC6CCC5C6)cc4)c4ccc5c(c4)oc4cccc(-c6ccccc6)c45)c3)cc2)cc1 ; Cc1cc(C)c2c(c1)oc1cc(N(c3ccc(C4CC5CCC4C5)cc3)c3cccc(-c4ccc(-c5ccccc5-c5ccccc5)cc4)c3)ccc12]

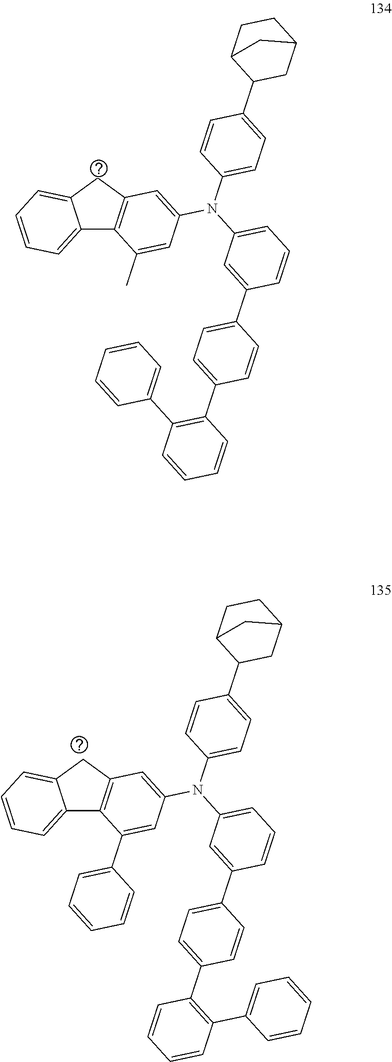 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2 ; c1ccc(-c2ccccc2-c2ccc(-c3cccc(N(c4ccc(C5CC6CCC5C6)cc4)c4cc5c(c(-c6ccccc6)c4)-c4ccccc4C5)c3)cc2)cc1]

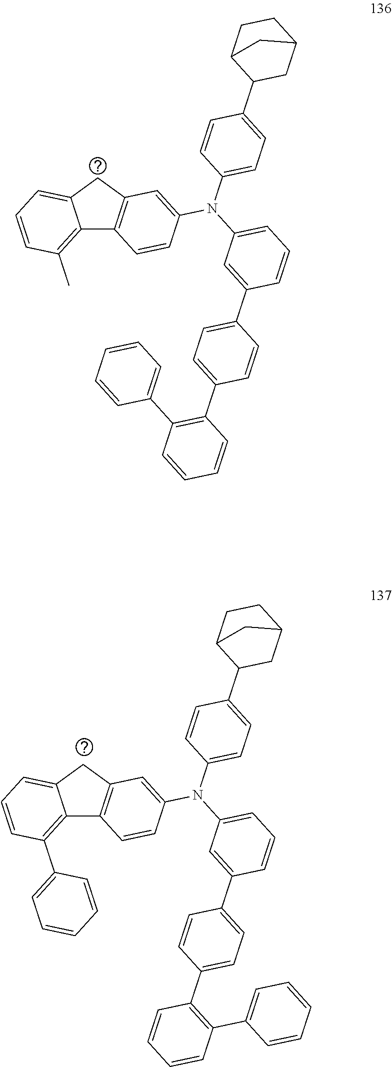 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2 ; c1ccc(-c2ccccc2-c2ccc(-c3cccc(N(c4ccc(C5CC6CCC5C6)cc4)c4ccc5c(c4)Cc4cccc(-c6ccccc6)c4-5)c3)cc2)cc1]

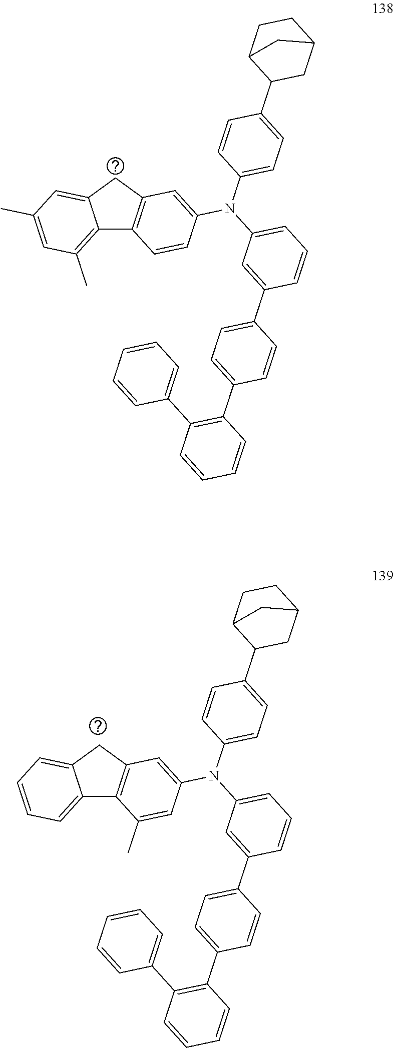 [SMILES: Cc1cc(C)c2c(c1)Cc1cc(N(c3ccc(C4CC5CCC4C5)cc3)c3cccc(-c4ccc(-c5ccccc5-c5ccccc5)cc4)c3)ccc1-2 ; Cc1cc(N(c2ccc(C3CC4CCC3C4)cc2)c2cccc(-c3ccc(-c4ccccc4-c4ccccc4)cc3)c2)cc2c1-c1ccccc1C2]

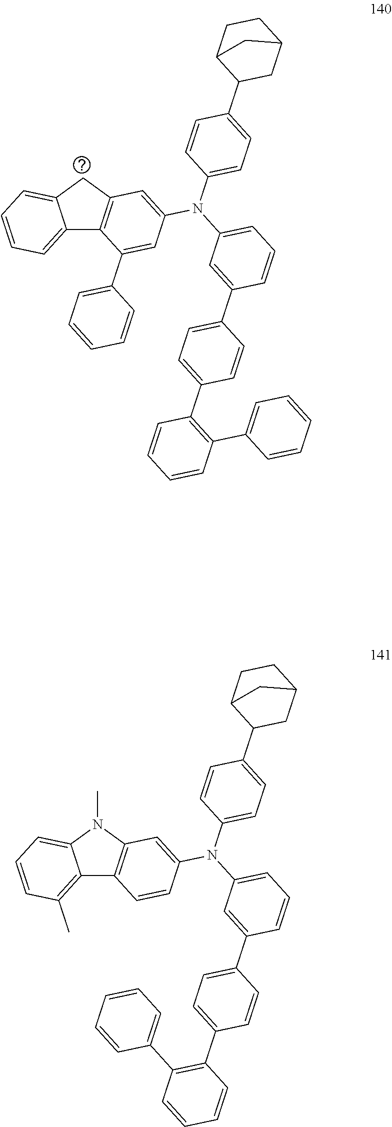 [SMILES: c1ccc(-c2ccccc2-c2ccc(-c3cccc(N(c4ccc(C5CC6CCC5C6)cc4)c4cc5c(c(-c6ccccc6)c4)-c4ccccc4C5)c3)cc2)cc1 ; Cc1cccc2c1c1ccc(N(c3ccc(C4CC5CCC4C5)cc3)c3cccc(-c4ccc(-c5ccccc5-c5ccccc5)cc4)c3)cc1n2C]

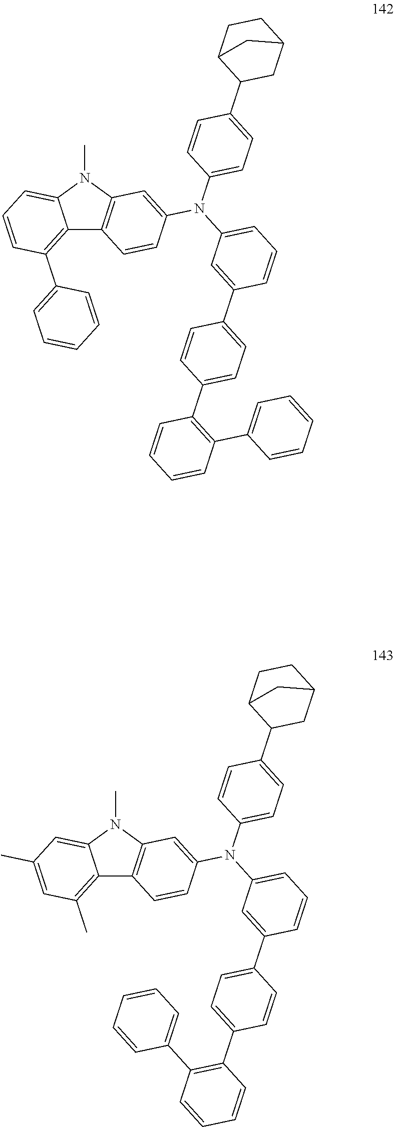 [SMILES: Cn1c2cc(N(c3ccc(C4CC5CCC4C5)cc3)c3cccc(-c4ccc(-c5ccccc5-c5ccccc5)cc4)c3)ccc2c2c(-c3ccccc3)cccc21 ; Cc1cc(C)c2c3ccc(N(c4ccc(C5CC6CCC5C6)cc4)c4cccc(-c5ccc(-c6ccccc6-c6ccccc6)cc5)c4)cc3n(C)c2c1]

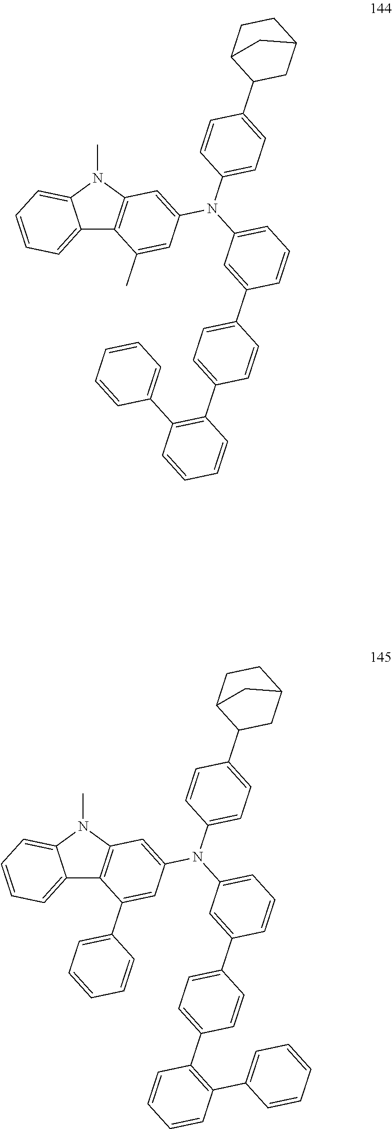 [SMILES: Cc1cc(N(c2ccc(C3CC4CCC3C4)cc2)c2cccc(-c3ccc(-c4ccccc4-c4ccccc4)cc3)c2)cc2c1c1ccccc1n2C ; Cn1c2ccccc2c2c(-c3ccccc3)cc(N(c3ccc(C4CC5CCC4C5)cc3)c3cccc(-c4ccc(-c5ccccc5-c5ccccc5)cc4)c3)cc21]

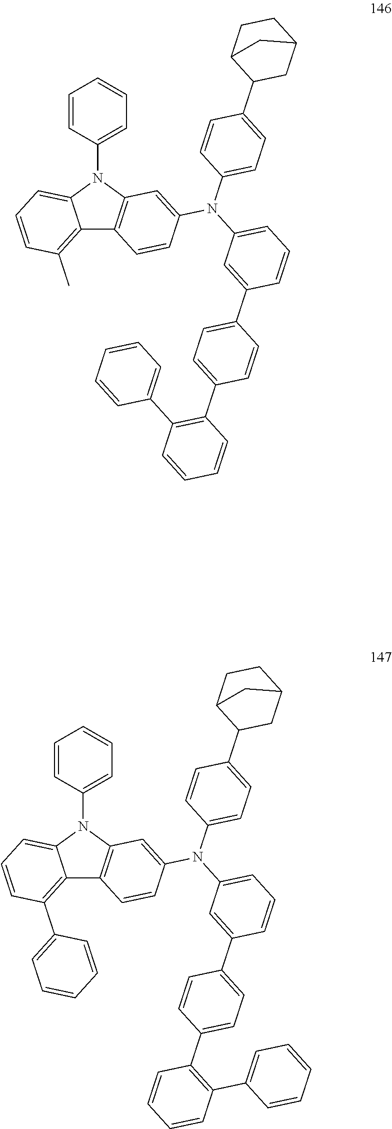 [SMILES: Cc1cccc2c1c1ccc(N(c3ccc(C4CC5CCC4C5)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C5CC6CCC5C6)cc4)c4ccc5c6c(-c7ccccc7)cccc6n(-c6ccccc6)c5c4)c3)cc2)cc1]

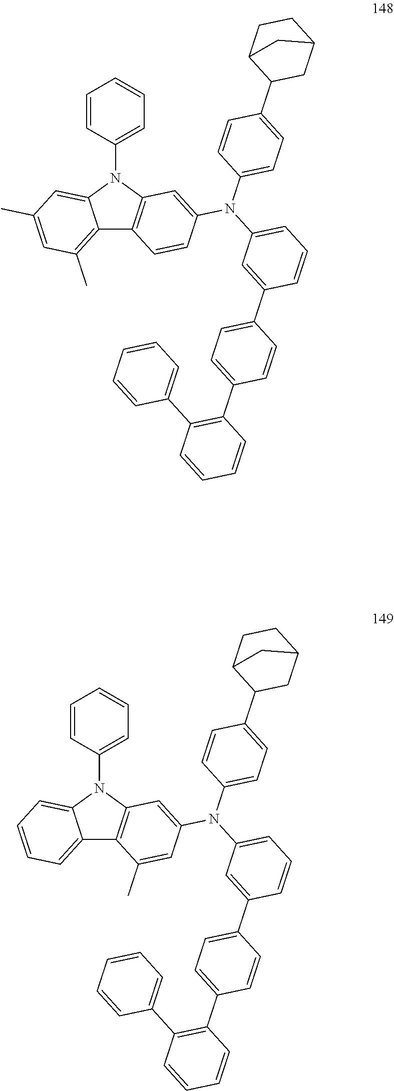 [SMILES: Cc1cc(C)c2c3ccc(N(c4ccc(C5CC6CCC5C6)cc4)c4cccc(-c5ccc(-c6ccccc6-c6ccccc6)cc5)c4)cc3n(-c3ccccc3)c2c1 ; Cc1cc(N(c2ccc(C3CC4CCC3C4)cc2)c2cccc(-c3ccc(-c4ccccc4-c4ccccc4)cc3)c2)cc2c1c1ccccc1n2-c1ccccc1]

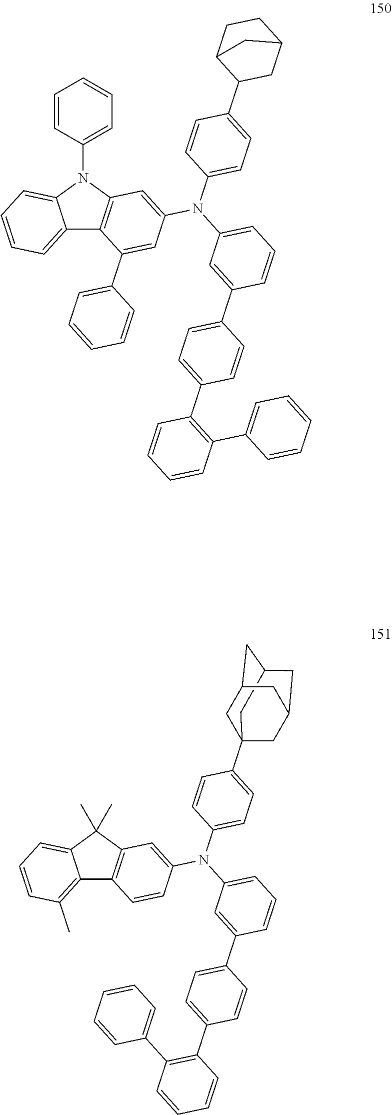 [SMILES: c1ccc(-c2ccccc2-c2ccc(-c3cccc(N(c4ccc(C5CC6CCC5C6)cc4)c4cc(-c5ccccc5)c5c6ccccc6n(-c6ccccc6)c5c4)c3)cc2)cc1 ; Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(C)C]

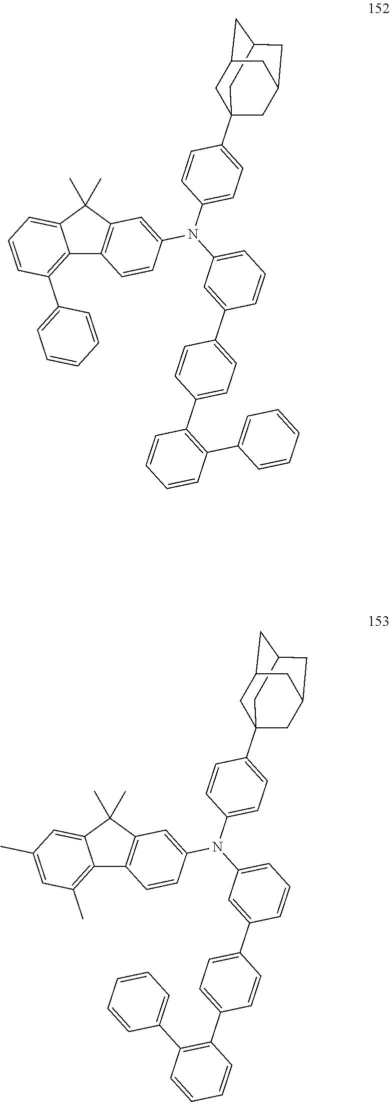 [SMILES: CC1(C)c2cc(N(c3ccc(C45CC6CC(CC(C6)C4)C5)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C45CC6CC(CC(C6)C4)C5)cc3)c3cccc(-c4ccc(-c5ccccc5-c5ccccc5)cc4)c3)ccc1-2]

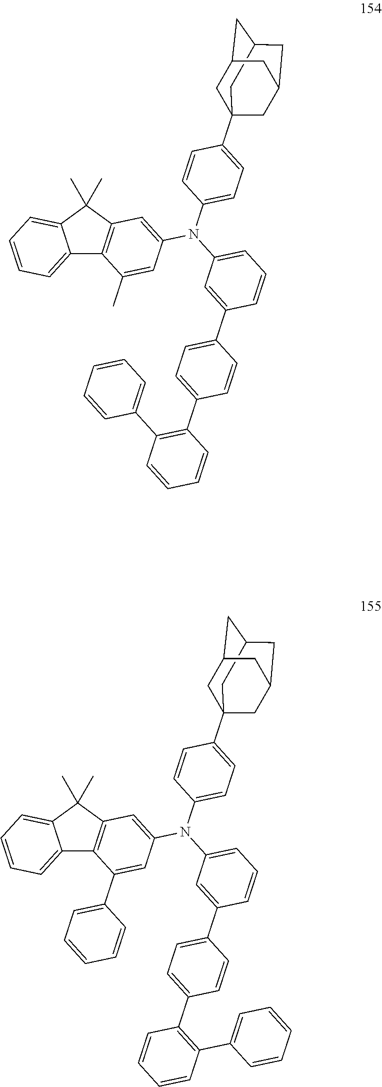 [SMILES: Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C45CC6CC(CC(C6)C4)C5)cc3)c3cccc(-c4ccc(-c5ccccc5-c5ccccc5)cc4)c3)cc21]

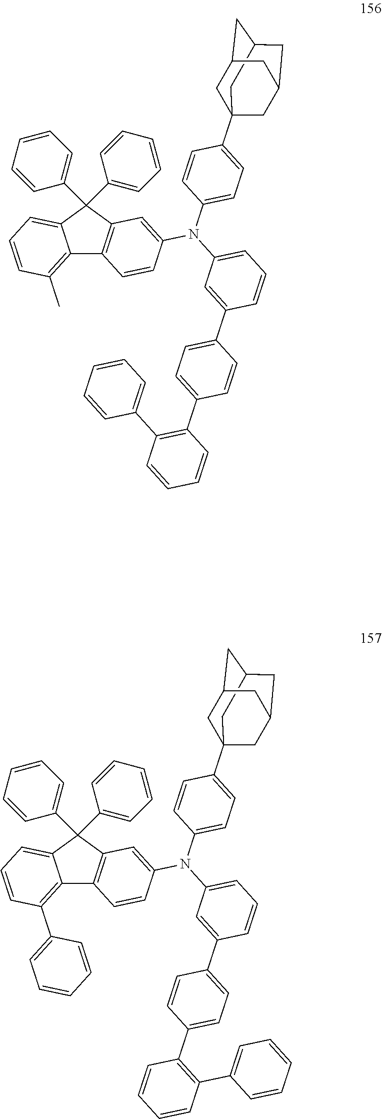 [SMILES: Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C56CC7CC(CC(C7)C5)C6)cc4)c4ccc5c(c4)C(c4ccccc4)(c4ccccc4)c4cccc(-c6ccccc6)c4-5)c3)cc2)cc1]

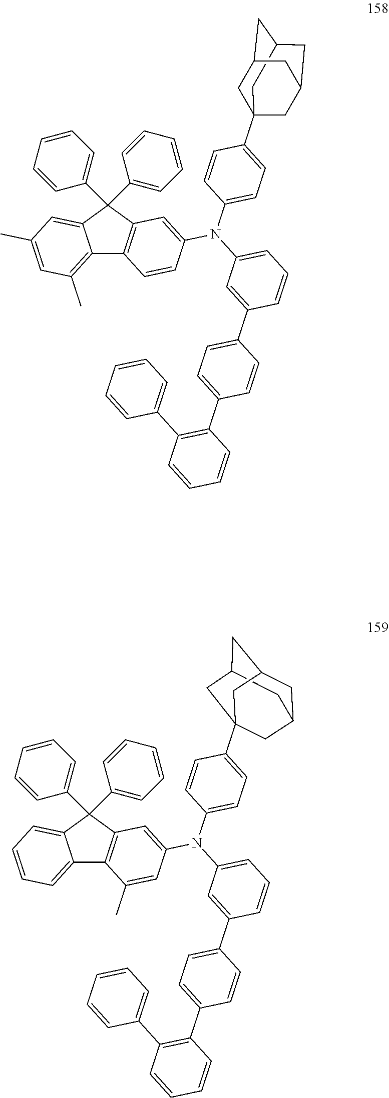 [SMILES: Cc1cc(C)c2c(c1)C(c1ccccc1)(c1ccccc1)c1cc(N(c3ccc(C45CC6CC(CC(C6)C4)C5)cc3)c3cccc(-c4ccc(-c5ccccc5-c5ccccc5)cc4)c3)ccc1-2 ; Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(c1ccccc1)c1ccccc1]

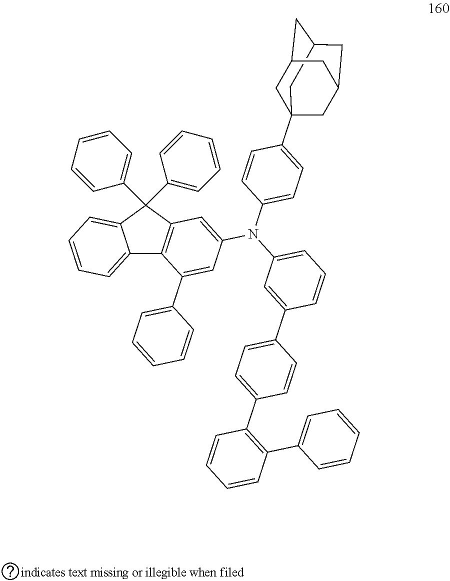 [SMILES: c1ccc(-c2ccccc2-c2ccc(-c3cccc(N(c4ccc(C56CC7CC(CC(C7)C5)C6)cc4)c4cc(-c5ccccc5)c5c(c4)C(c4ccccc4)(c4ccccc4)c4ccccc4-5)c3)cc2)cc1]

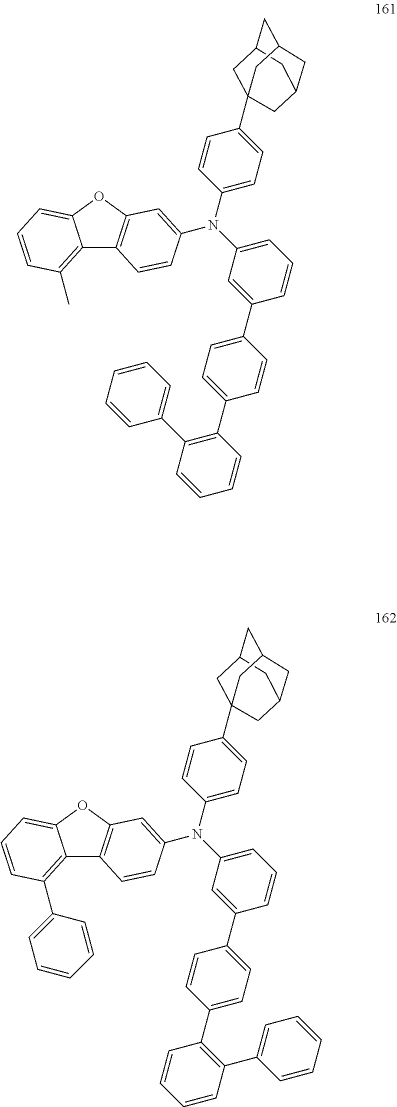 [SMILES: Cc1cccc2oc3cc(N(c4ccc(C56CC7CC(CC(C7)C5)C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C56CC7CC(CC(C7)C5)C6)cc4)c4ccc5c(c4)oc4cccc(-c6ccccc6)c45)c3)cc2)cc1]

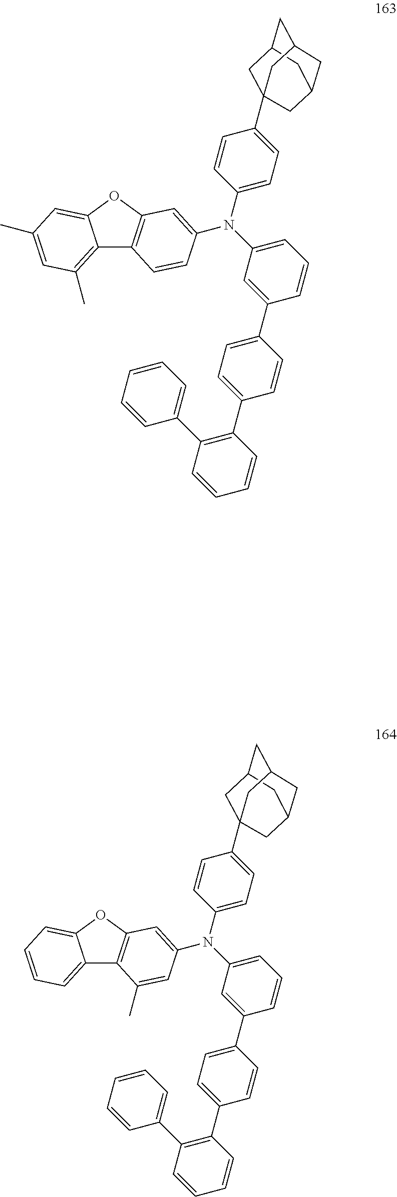 [SMILES: Cc1cc(C)c2c(c1)oc1cc(N(c3ccc(C45CC6CC(CC(C6)C4)C5)cc3)c3cccc(-c4ccc(-c5ccccc5-c5ccccc5)cc4)c3)ccc12 ; Cc1cc(N(c2ccc(C34CC5CC(CC(C5)C3)C4)cc2)c2cccc(-c3ccc(-c4ccccc4-c4ccccc4)cc3)c2)cc2oc3ccccc3c12]

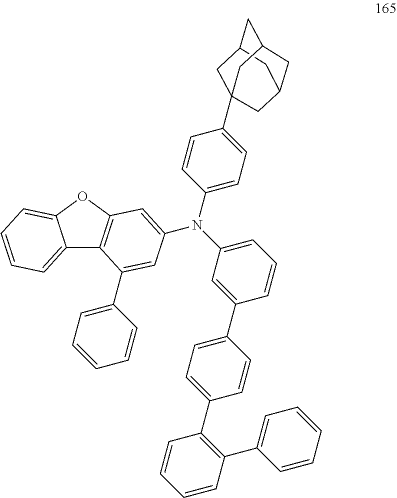 [SMILES: c1ccc(-c2ccccc2-c2ccc(-c3cccc(N(c4ccc(C56CC7CC(CC(C7)C5)C6)cc4)c4cc(-c5ccccc5)c5c(c4)oc4ccccc45)c3)cc2)cc1]

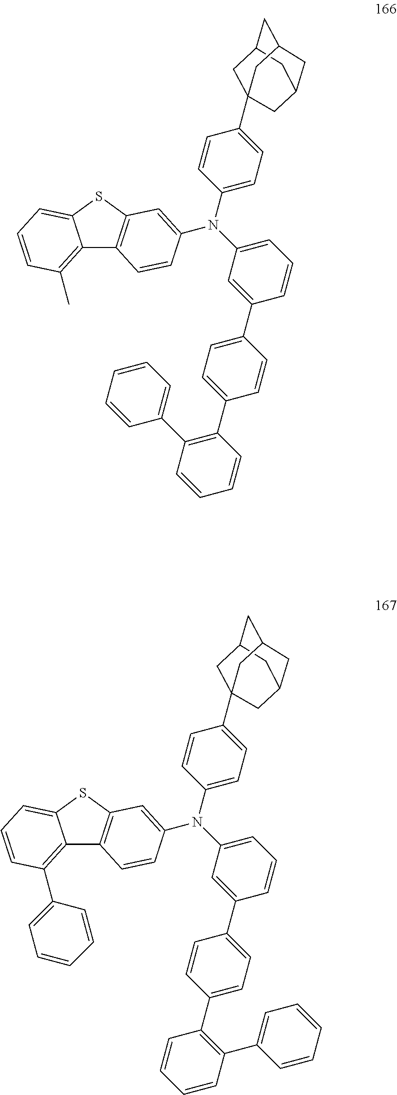 [SMILES: Cc1cccc2sc3cc(N(c4ccc(C56CC7CC(CC(C7)C5)C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C56CC7CC(CC(C7)C5)C6)cc4)c4ccc5c(c4)sc4cccc(-c6ccccc6)c45)c3)cc2)cc1]

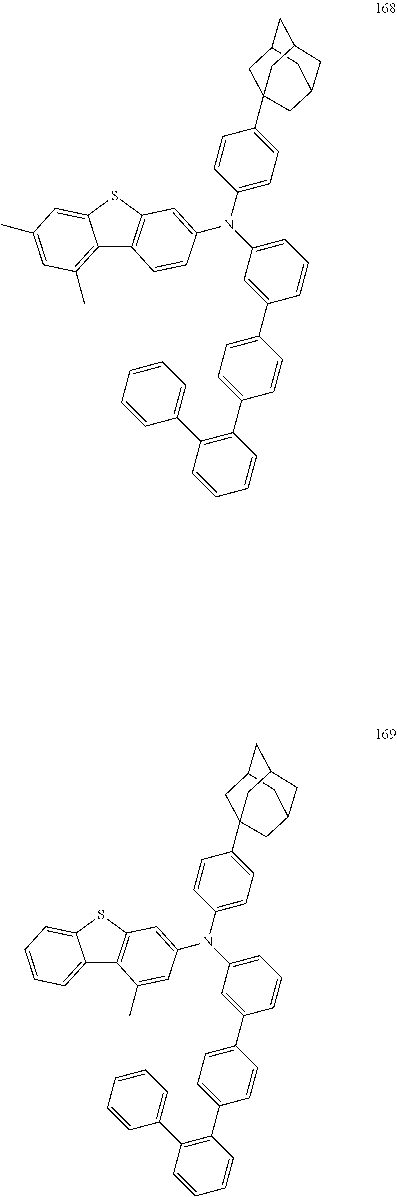 [SMILES: Cc1cc(C)c2c(c1)sc1cc(N(c3ccc(C45CC6CC(CC(C6)C4)C5)cc3)c3cccc(-c4ccc(-c5ccccc5-c5ccccc5)cc4)c3)ccc12 ; Cc1cc(N(c2ccc(C34CC5CC(CC(C5)C3)C4)cc2)c2cccc(-c3ccc(-c4ccccc4-c4ccccc4)cc3)c2)cc2sc3ccccc3c12]

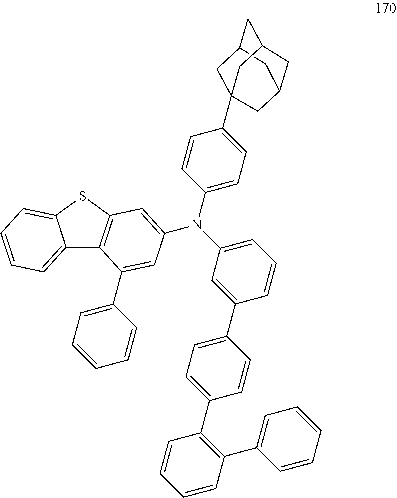 [SMILES: c1ccc(-c2ccccc2-c2ccc(-c3cccc(N(c4ccc(C56CC7CC(CC(C7)C5)C6)cc4)c4cc(-c5ccccc5)c5c(c4)sc4ccccc45)c3)cc2)cc1]

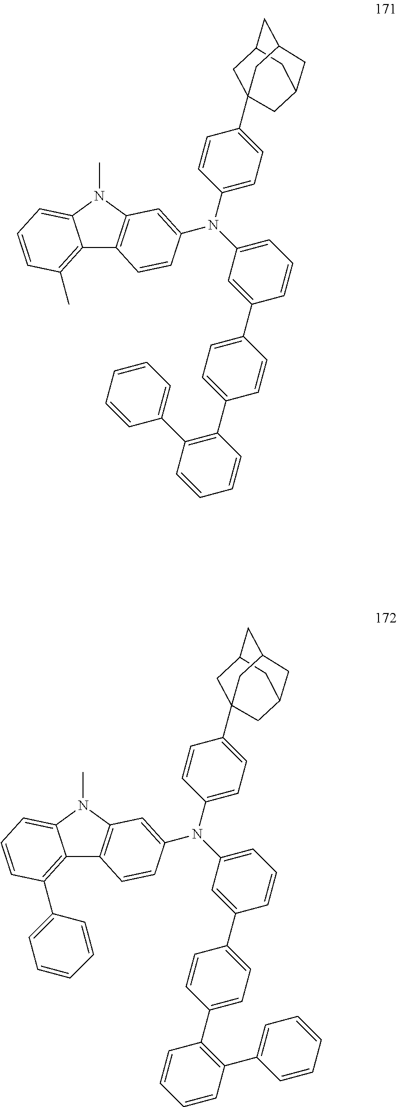 [SMILES: Cc1cccc2c1c1ccc(N(c3ccc(C45CC6CC(CC(C6)C4)C5)cc3)c3cccc(-c4ccc(-c5ccccc5-c5ccccc5)cc4)c3)cc1n2C ; Cn1c2cc(N(c3ccc(C45CC6CC(CC(C6)C4)C5)cc3)c3cccc(-c4ccc(-c5ccccc5-c5ccccc5)cc4)c3)ccc2c2c(-c3ccccc3)cccc21]

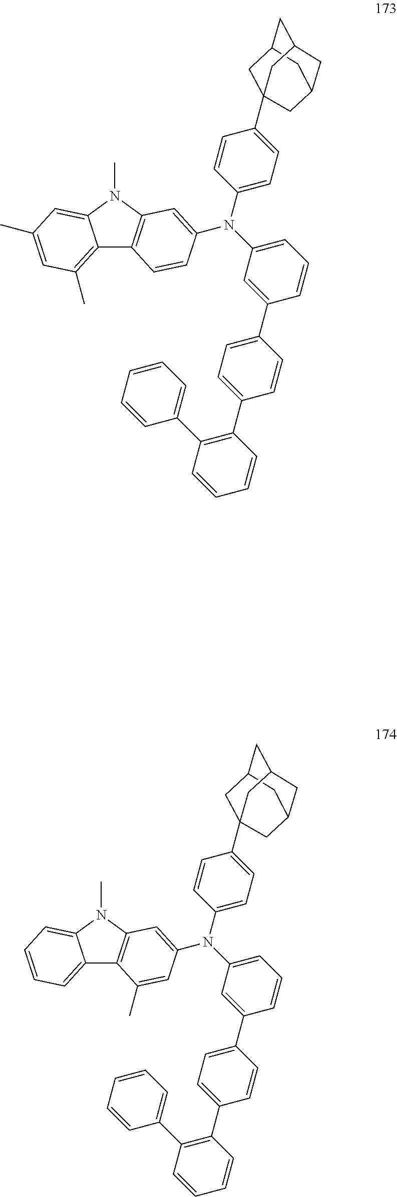 [SMILES: Cc1cc(C)c2c3ccc(N(c4ccc(C56CC7CC(CC(C7)C5)C6)cc4)c4cccc(-c5ccc(-c6ccccc6-c6ccccc6)cc5)c4)cc3n(C)c2c1 ; Cc1cc(N(c2ccc(C34CC5CC(CC(C5)C3)C4)cc2)c2cccc(-c3ccc(-c4ccccc4-c4ccccc4)cc3)c2)cc2c1c1ccccc1n2C]

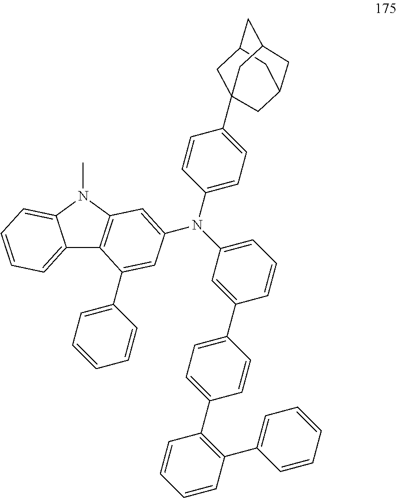 [SMILES: Cn1c2ccccc2c2c(-c3ccccc3)cc(N(c3ccc(C45CC6CC(CC(C6)C4)C5)cc3)c3cccc(-c4ccc(-c5ccccc5-c5ccccc5)cc4)c3)cc21]

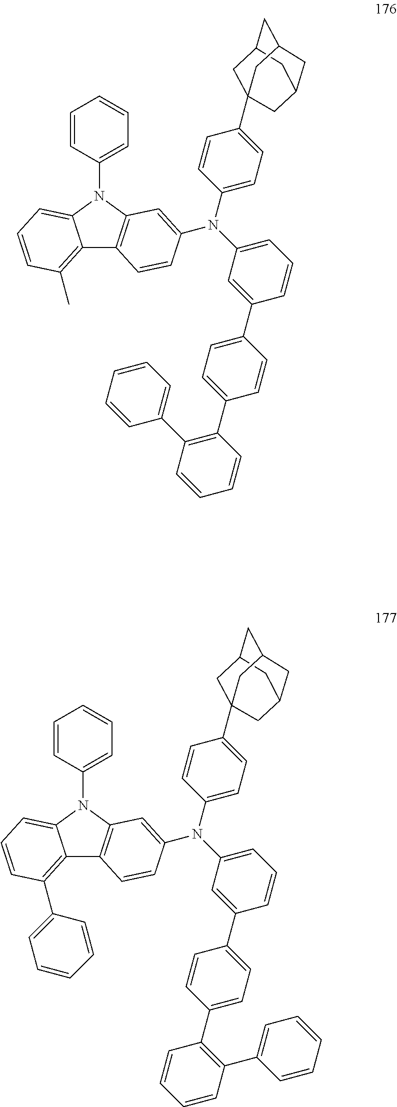 [SMILES: Cc1cccc2c1c1ccc(N(c3ccc(C45CC6CC(CC(C6)C4)C5)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C56CC7CC(CC(C7)C5)C6)cc4)c4ccc5c6c(-c7ccccc7)cccc6n(-c6ccccc6)c5c4)c3)cc2)cc1]

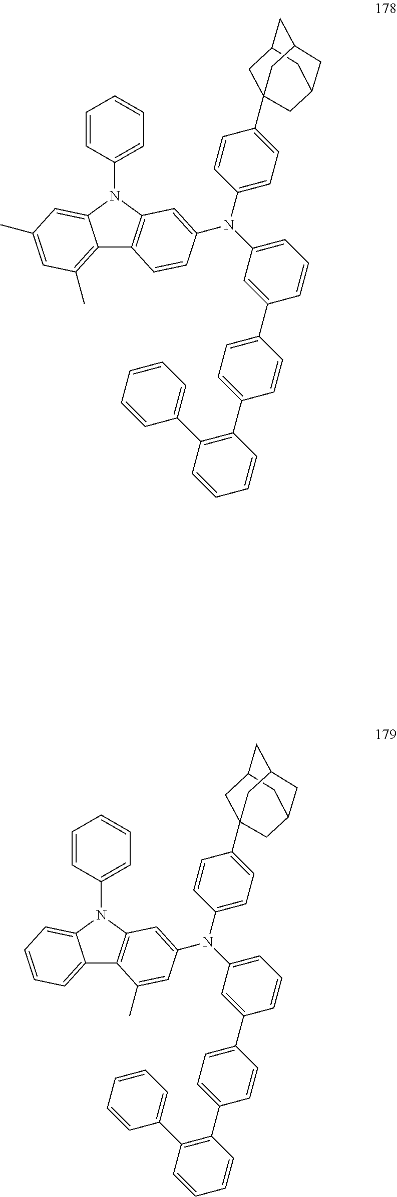 [SMILES: Cc1cc(C)c2c3ccc(N(c4ccc(C56CC7CC(CC(C7)C5)C6)cc4)c4cccc(-c5ccc(-c6ccccc6-c6ccccc6)cc5)c4)cc3n(-c3ccccc3)c2c1 ; Cc1cc(N(c2ccc(C34CC5CC(CC(C5)C3)C4)cc2)c2cccc(-c3ccc(-c4ccccc4-c4ccccc4)cc3)c2)cc2c1c1ccccc1n2-c1ccccc1]

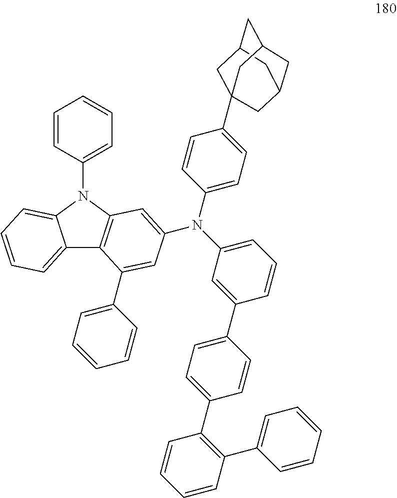 [SMILES: c1ccc(-c2ccccc2-c2ccc(-c3cccc(N(c4ccc(C56CC7CC(CC(C7)C5)C6)cc4)c4cc(-c5ccccc5)c5c6ccccc6n(-c6ccccc6)c5c4)c3)cc2)cc1]

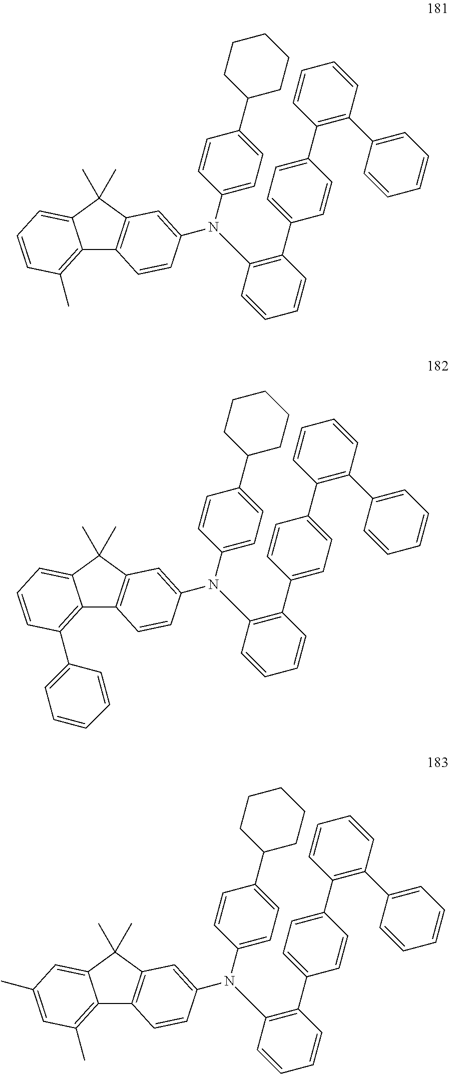 [SMILES: Cc1cccc2c1-c1ccc(N(c3ccc(C4CCCCC4)cc3)c3ccccc3-c3ccc(-c4ccccc4-c4ccccc4)cc3)cc1C2(C)C ; CC1(C)c2cc(N(c3ccc(C4CCCCC4)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CCCCC4)cc3)c3ccccc3-c3ccc(-c4ccccc4-c4ccccc4)cc3)ccc1-2]

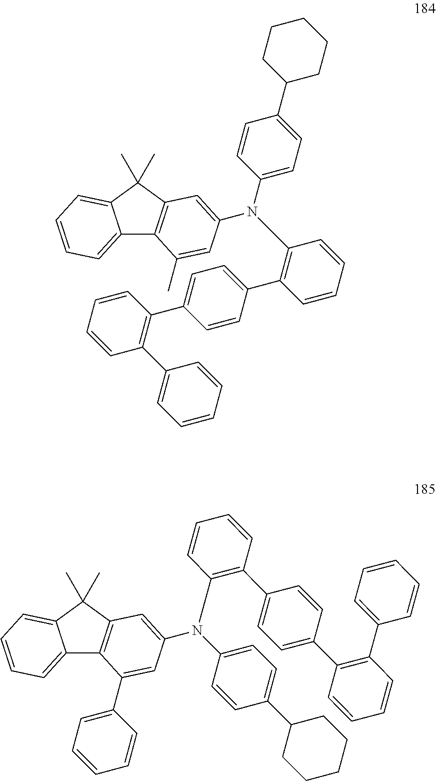 [SMILES: Cc1cc(N(c2ccc(C3CCCCC3)cc2)c2ccccc2-c2ccc(-c3ccccc3-c3ccccc3)cc2)cc2c1-c1ccccc1C2(C)C ; CC1(C)c2ccccc2-c2c(-c3ccccc3)cc(N(c3ccc(C4CCCCC4)cc3)c3ccccc3-c3ccc(-c4ccccc4-c4ccccc4)cc3)cc21]

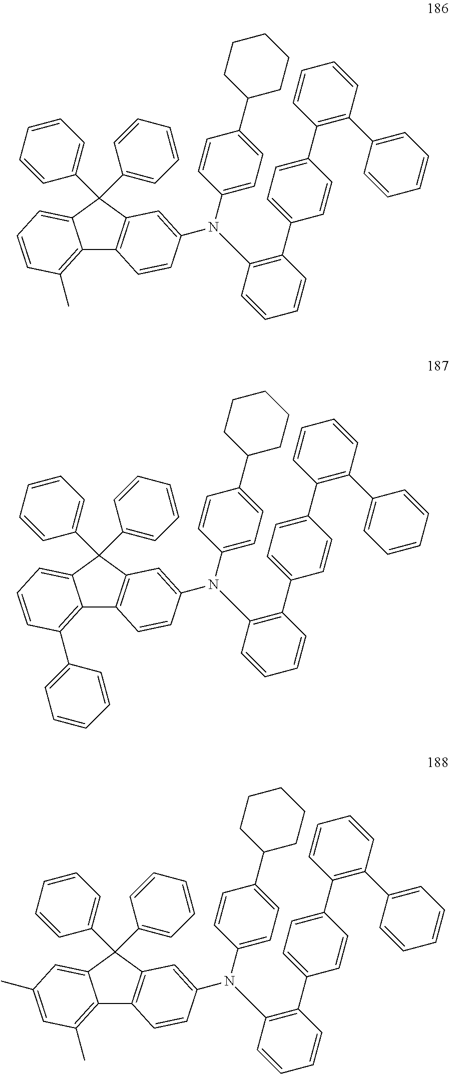 [SMILES: Cc1cccc2c1-c1ccc(N(c3ccc(C4CCCCC4)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C4CCCCC4)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C4CCCCC4)cc3)c3ccccc3-c3ccc(-c4ccccc4-c4ccccc4)cc3)ccc1-2]

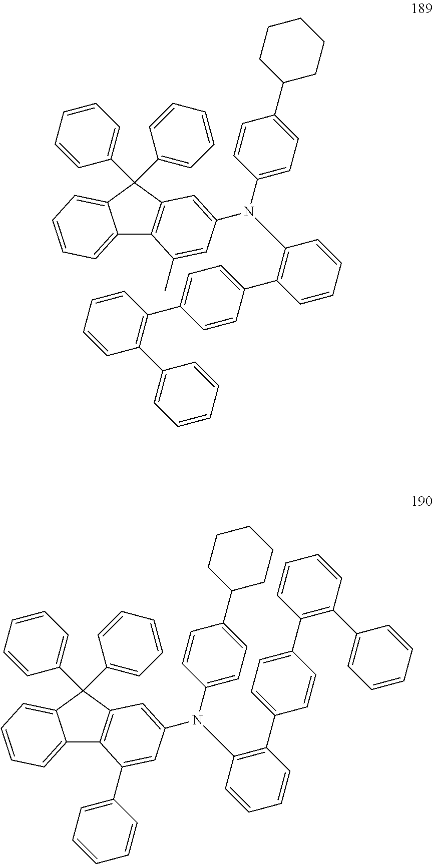 [SMILES: Cc1cc(N(c2ccc(C3CCCCC3)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C4CCCCC4)cc3)c3cc(-c4ccccc4)c4c(c3)C(c3ccccc3)(c3ccccc3)c3ccccc3-4)cc2)cc1]

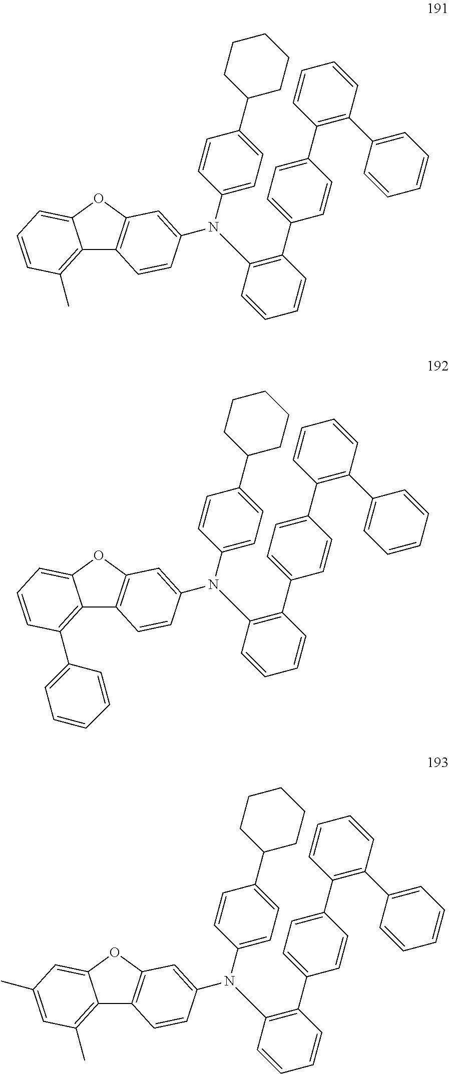 [SMILES: Cc1cccc2oc3cc(N(c4ccc(C5CCCCC5)cc4)c4ccccc4-c4ccc(-c5ccccc5-c5ccccc5)cc4)ccc3c12 ; c1ccc(-c2ccccc2-c2ccc(-c3ccccc3N(c3ccc(C4CCCCC4)cc3)c3ccc4c(c3)oc3cccc(-c5ccccc5)c34)cc2)cc1 ; Cc1cc(C)c2c(c1)oc1cc(N(c3ccc(C4CCCCC4)cc3)c3ccccc3-c3ccc(-c4ccccc4-c4ccccc4)cc3)ccc12]

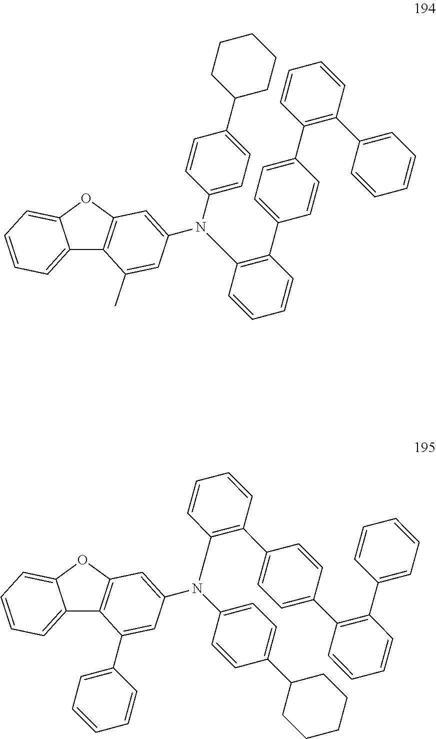 [SMILES: Cc1cc(N(c2ccc(C3CCCCC3)cc2)c2ccccc2-c2ccc(-c3ccccc3-c3ccccc3)cc2)cc2oc3ccccc3c12 ; c1ccc(-c2ccccc2-c2ccc(-c3ccccc3N(c3ccc(C4CCCCC4)cc3)c3cc(-c4ccccc4)c4c(c3)oc3ccccc34)cc2)cc1]

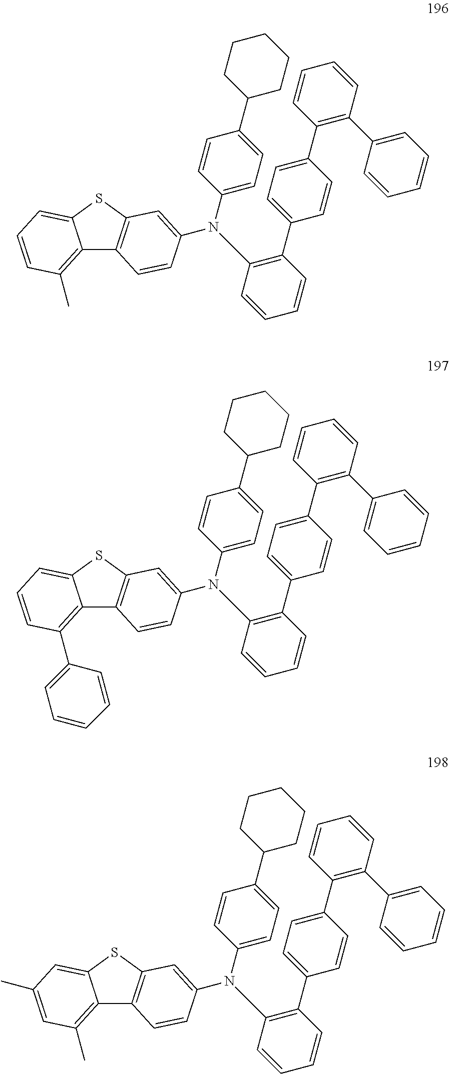 [SMILES: Cc1cccc2sc3cc(N(c4ccc(C5CCCCC5)cc4)c4ccccc4-c4ccc(-c5ccccc5-c5ccccc5)cc4)ccc3c12 ; c1ccc(-c2ccccc2-c2ccc(-c3ccccc3N(c3ccc(C4CCCCC4)cc3)c3ccc4c(c3)sc3cccc(-c5ccccc5)c34)cc2)cc1 ; Cc1cc(C)c2c(c1)sc1cc(N(c3ccc(C4CCCCC4)cc3)c3ccccc3-c3ccc(-c4ccccc4-c4ccccc4)cc3)ccc12]

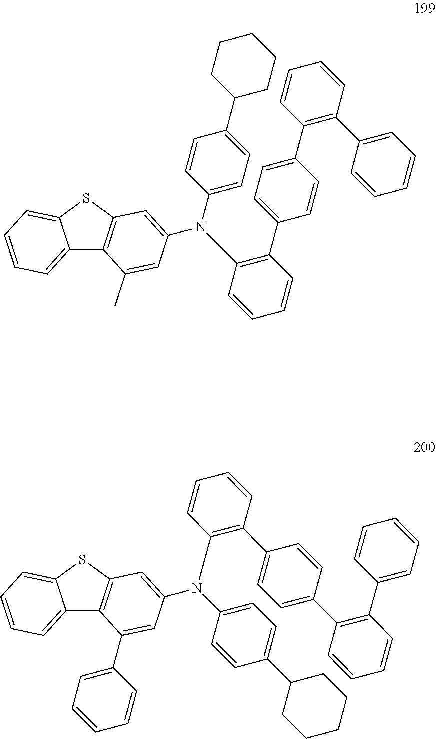 [SMILES: Cc1cc(N(c2ccc(C3CCCCC3)cc2)c2ccccc2-c2ccc(-c3ccccc3-c3ccccc3)cc2)cc2sc3ccccc3c12 ; c1ccc(-c2ccccc2-c2ccc(-c3ccccc3N(c3ccc(C4CCCCC4)cc3)c3cc(-c4ccccc4)c4c(c3)sc3ccccc34)cc2)cc1]

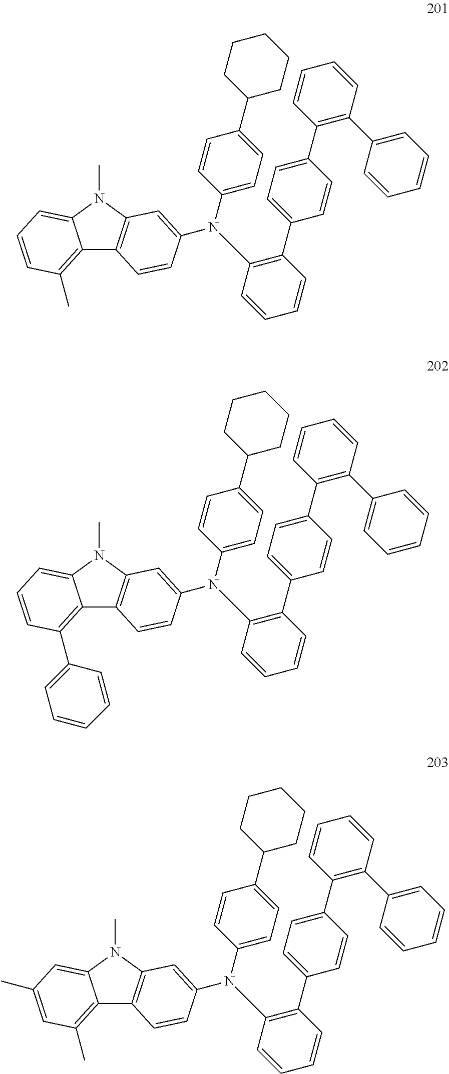 [SMILES: Cc1cccc2c1c1ccc(N(c3ccc(C4CCCCC4)cc3)c3ccccc3-c3ccc(-c4ccccc4-c4ccccc4)cc3)cc1n2C ; Cn1c2cc(N(c3ccc(C4CCCCC4)cc3)c3ccccc3-c3ccc(-c4ccccc4-c4ccccc4)cc3)ccc2c2c(-c3ccccc3)cccc21 ; Cc1cc(C)c2c3ccc(N(c4ccc(C5CCCCC5)cc4)c4ccccc4-c4ccc(-c5ccccc5-c5ccccc5)cc4)cc3n(C)c2c1]

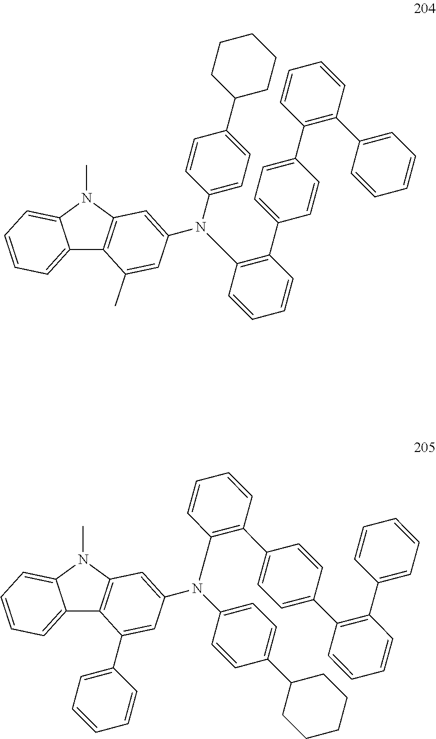 [SMILES: Cc1cc(N(c2ccc(C3CCCCC3)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C4CCCCC4)cc3)c3ccccc3-c3ccc(-c4ccccc4-c4ccccc4)cc3)cc21]

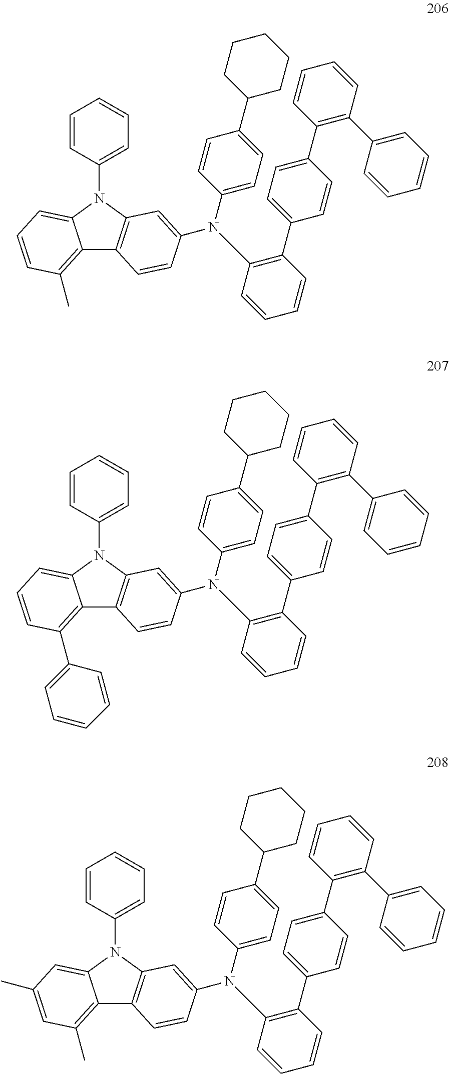 [SMILES: Cc1cccc2c1c1ccc(N(c3ccc(C4CCCCC4)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C4CCCCC4)cc3)c3ccc4c5c(-c6ccccc6)cccc5n(-c5ccccc5)c4c3)cc2)cc1 ; Cc1cc(C)c2c3ccc(N(c4ccc(C5CCCCC5)cc4)c4ccccc4-c4ccc(-c5ccccc5-c5ccccc5)cc4)cc3n(-c3ccccc3)c2c1]

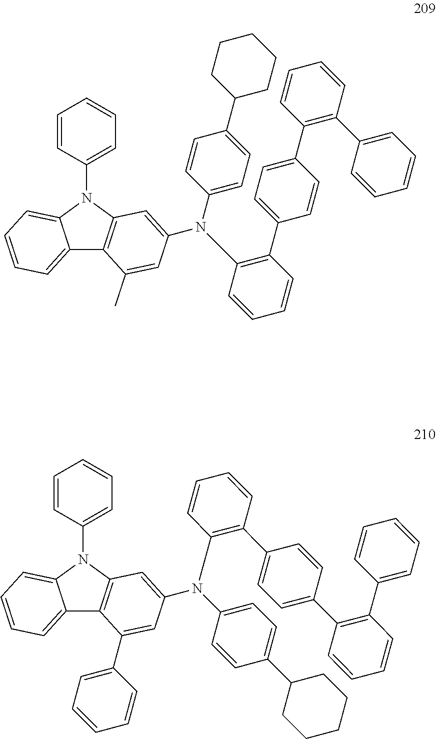 [SMILES: Cc1cc(N(c2ccc(C3CCCCC3)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CCCCC4)cc3)c3cc(-c4ccccc4)c4c5ccccc5n(-c5ccccc5)c4c3)cc2)cc1]

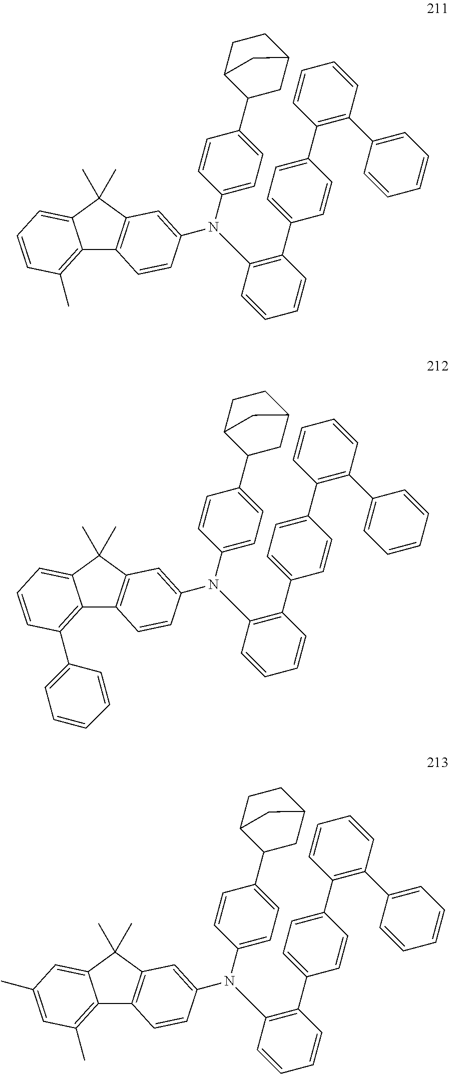 [SMILES: Cc1cccc2c1-c1ccc(N(c3ccc(C4CC5CCC4C5)cc3)c3ccccc3-c3ccc(-c4ccccc4-c4ccccc4)cc3)cc1C2(C)C ; CC1(C)c2cc(N(c3ccc(C4CC5CCC4C5)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CC5CCC4C5)cc3)c3ccccc3-c3ccc(-c4ccccc4-c4ccccc4)cc3)ccc1-2]

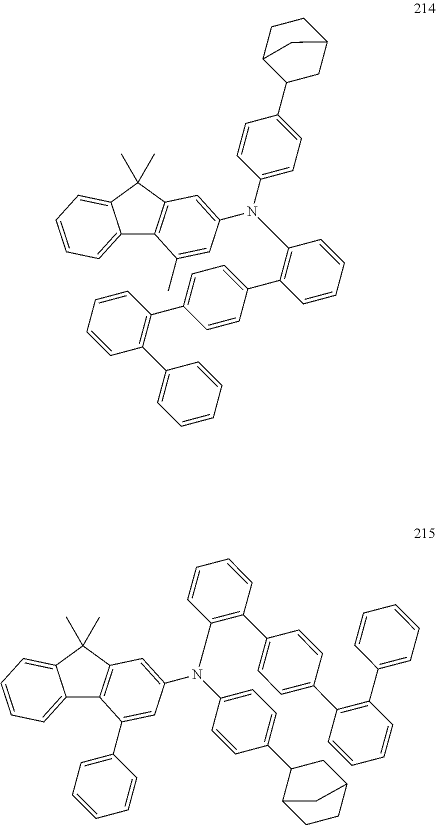 [SMILES: Cc1cc(N(c2ccc(C3CC4CCC3C4)cc2)c2ccccc2-c2ccc(-c3ccccc3-c3ccccc3)cc2)cc2c1-c1ccccc1C2(C)C ; CC1(C)c2ccccc2-c2c(-c3ccccc3)cc(N(c3ccc(C4CC5CCC4C5)cc3)c3ccccc3-c3ccc(-c4ccccc4-c4ccccc4)cc3)cc21]

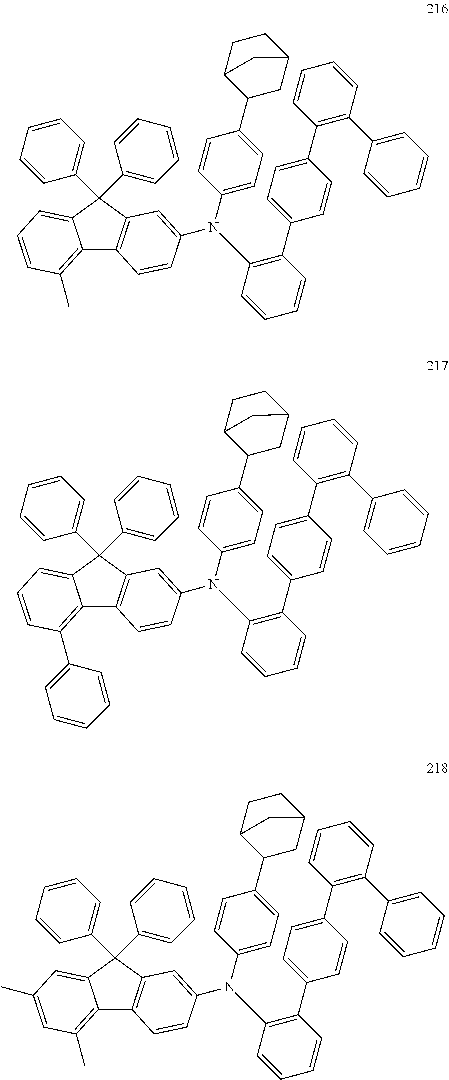 [SMILES: Cc1cccc2c1-c1ccc(N(c3ccc(C4CC5CCC4C5)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C4CC5CCC4C5)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C4CC5CCC4C5)cc3)c3ccccc3-c3ccc(-c4ccccc4-c4ccccc4)cc3)ccc1-2]

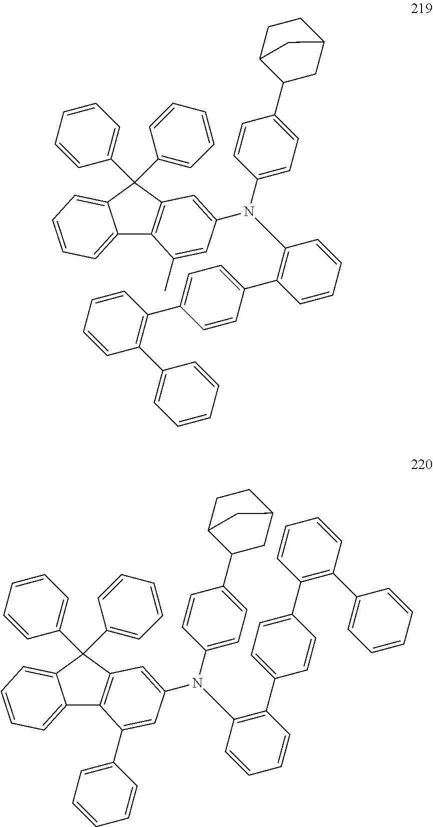 [SMILES: Cc1cc(N(c2ccc(C3CC4CCC3C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C4CC5CCC4C5)cc3)c3cc(-c4ccccc4)c4c(c3)C(c3ccccc3)(c3ccccc3)c3ccccc3-4)cc2)cc1]

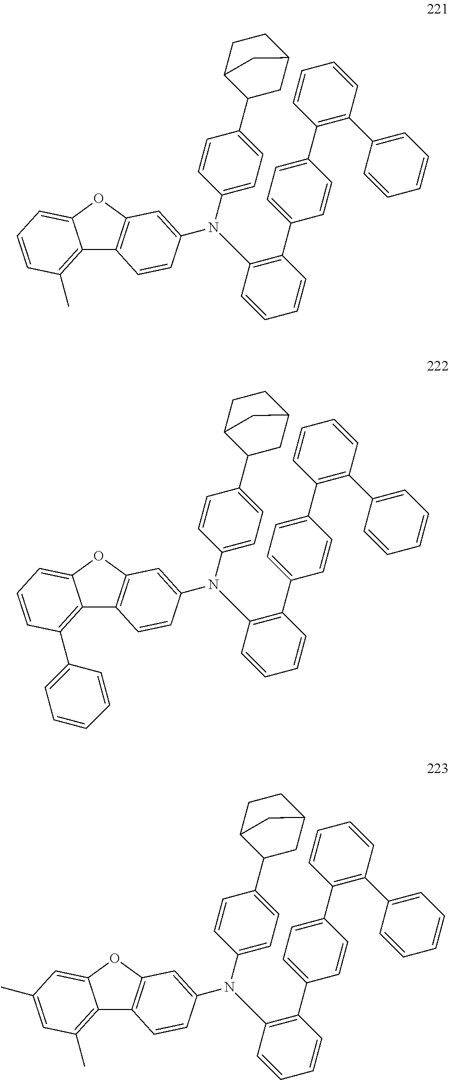 [SMILES: Cc1cccc2oc3cc(N(c4ccc(C5CC6CCC5C6)cc4)c4ccccc4-c4ccc(-c5ccccc5-c5ccccc5)cc4)ccc3c12 ; c1ccc(-c2ccccc2-c2ccc(-c3ccccc3N(c3ccc(C4CC5CCC4C5)cc3)c3ccc4c(c3)oc3cccc(-c5ccccc5)c34)cc2)cc1 ; Cc1cc(C)c2c(c1)oc1cc(N(c3ccc(C4CC5CCC4C5)cc3)c3ccccc3-c3ccc(-c4ccccc4-c4ccccc4)cc3)ccc12]

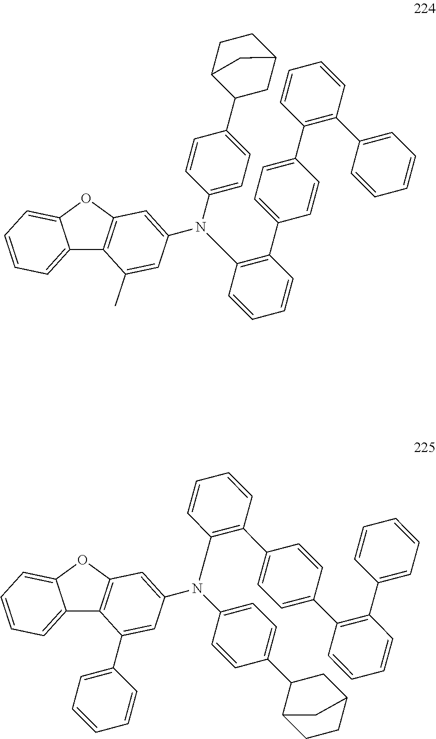 [SMILES: Cc1cc(N(c2ccc(C3CC4CCC3C4)cc2)c2ccccc2-c2ccc(-c3ccccc3-c3ccccc3)cc2)cc2oc3ccccc3c12 ; c1ccc(-c2ccccc2-c2ccc(-c3ccccc3N(c3ccc(C4CC5CCC4C5)cc3)c3cc(-c4ccccc4)c4c(c3)oc3ccccc34)cc2)cc1]

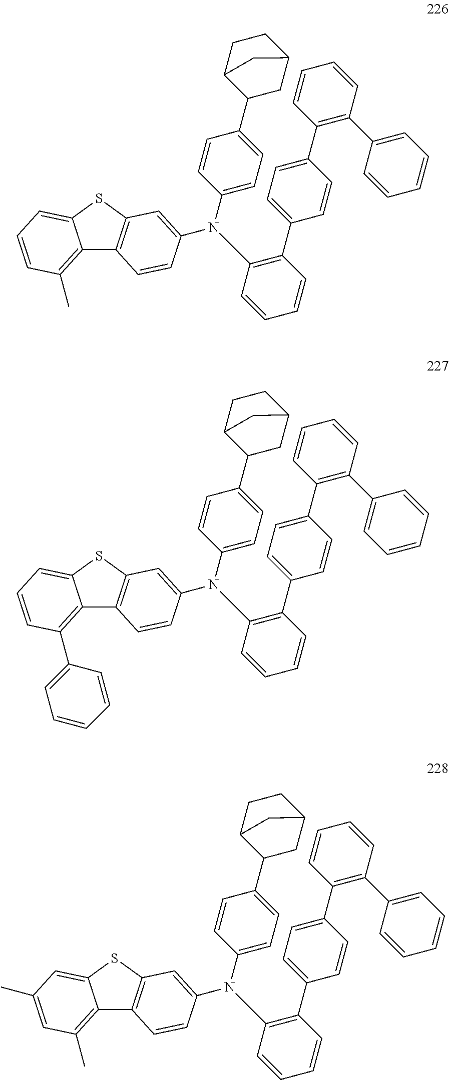 [SMILES: Cc1cccc2sc3cc(N(c4ccc(C5CC6CCC5C6)cc4)c4ccccc4-c4ccc(-c5ccccc5-c5ccccc5)cc4)ccc3c12 ; c1ccc(-c2ccccc2-c2ccc(-c3ccccc3N(c3ccc(C4CC5CCC4C5)cc3)c3ccc4c(c3)sc3cccc(-c5ccccc5)c34)cc2)cc1 ; Cc1cc(C)c2c(c1)sc1cc(N(c3ccc(C4CC5CCC4C5)cc3)c3ccccc3-c3ccc(-c4ccccc4-c4ccccc4)cc3)ccc12]

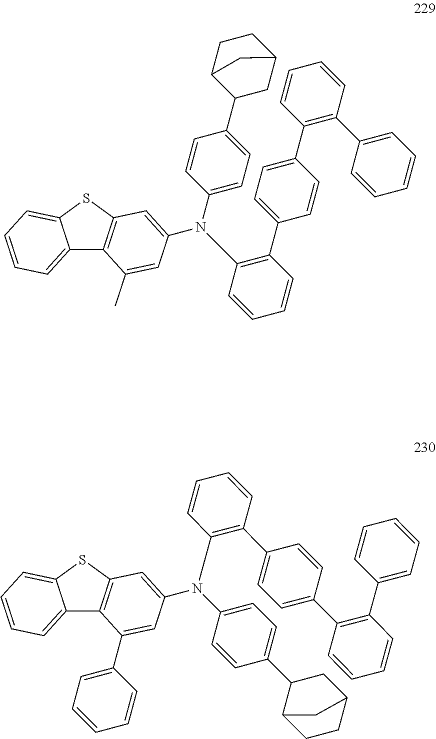 [SMILES: Cc1cc(N(c2ccc(C3CC4CCC3C4)cc2)c2ccccc2-c2ccc(-c3ccccc3-c3ccccc3)cc2)cc2sc3ccccc3c12 ; c1ccc(-c2ccccc2-c2ccc(-c3ccccc3N(c3ccc(C4CC5CCC4C5)cc3)c3cc(-c4ccccc4)c4c(c3)sc3ccccc34)cc2)cc1]

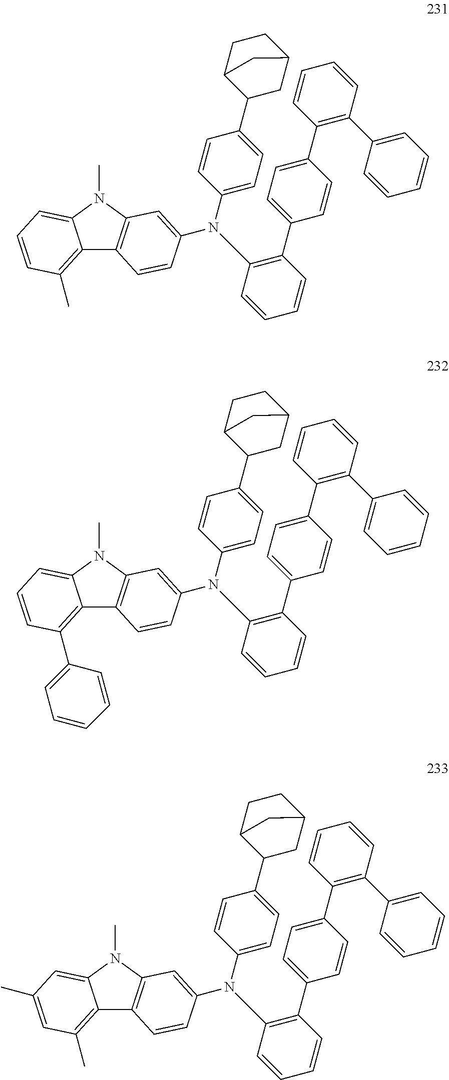 [SMILES: Cc1cccc2c1c1ccc(N(c3ccc(C4CC5CCC4C5)cc3)c3ccccc3-c3ccc(-c4ccccc4-c4ccccc4)cc3)cc1n2C ; Cn1c2cc(N(c3ccc(C4CC5CCC4C5)cc3)c3ccccc3-c3ccc(-c4ccccc4-c4ccccc4)cc3)ccc2c2c(-c3ccccc3)cccc21 ; Cc1cc(C)c2c3ccc(N(c4ccc(C5CC6CCC5C6)cc4)c4ccccc4-c4ccc(-c5ccccc5-c5ccccc5)cc4)cc3n(C)c2c1]

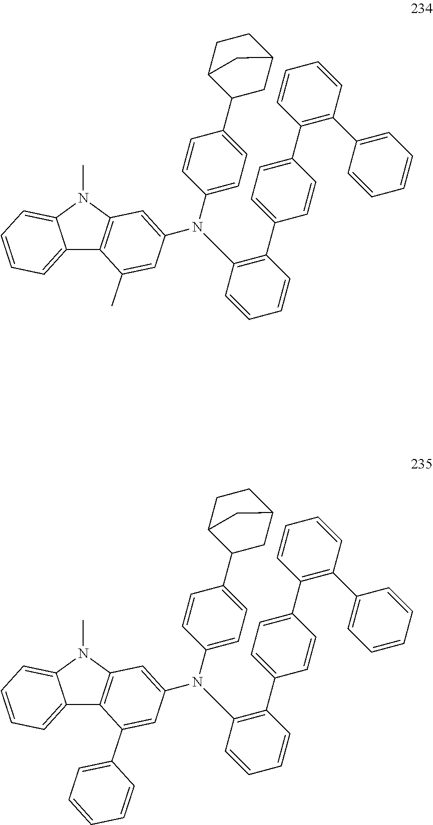 [SMILES: Cc1cc(N(c2ccc(C3CC4CCC3C4)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C4CC5CCC4C5)cc3)c3ccccc3-c3ccc(-c4ccccc4-c4ccccc4)cc3)cc21]

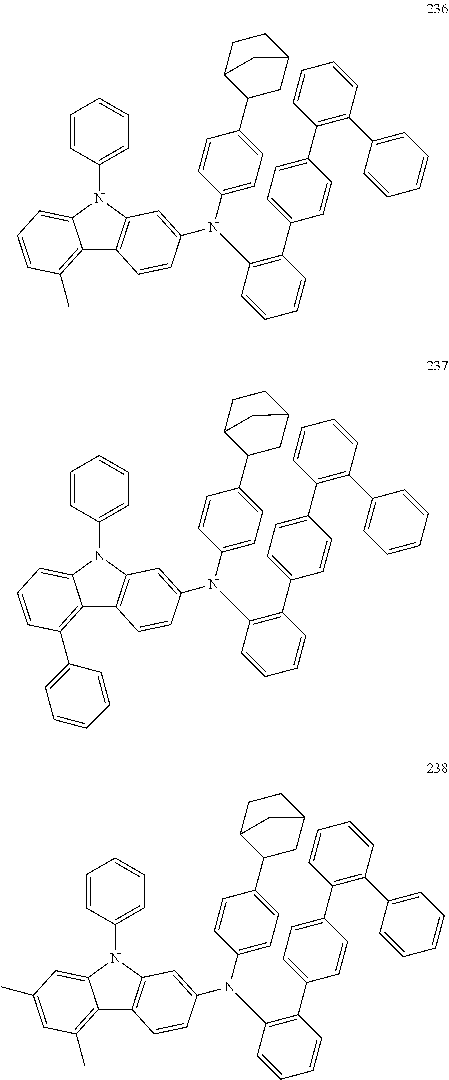 [SMILES: Cc1cccc2c1c1ccc(N(c3ccc(C4CC5CCC4C5)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C4CC5CCC4C5)cc3)c3ccc4c5c(-c6ccccc6)cccc5n(-c5ccccc5)c4c3)cc2)cc1 ; Cc1cc(C)c2c3ccc(N(c4ccc(C5CC6CCC5C6)cc4)c4ccccc4-c4ccc(-c5ccccc5-c5ccccc5)cc4)cc3n(-c3ccccc3)c2c1]

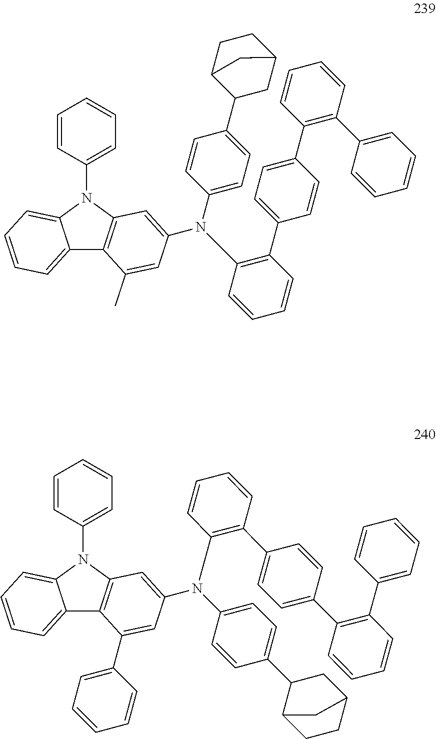 [SMILES: Cc1cc(N(c2ccc(C3CC4CCC3C4)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CC5CCC4C5)cc3)c3cc(-c4ccccc4)c4c5ccccc5n(-c5ccccc5)c4c3)cc2)cc1]

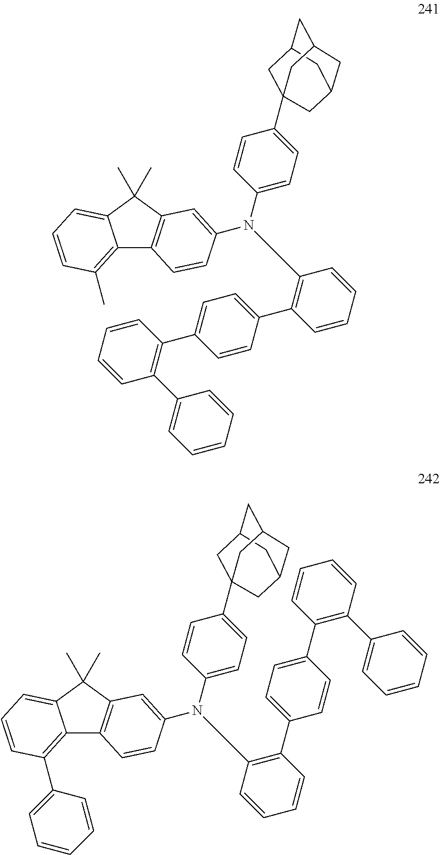 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(C)C ; CC1(C)c2cc(N(c3ccc(C45CC6CC(CC(C6)C4)C5)cc3)c3ccccc3-c3ccc(-c4ccccc4-c4ccccc4)cc3)ccc2-c2c(-c3ccccc3)cccc21]

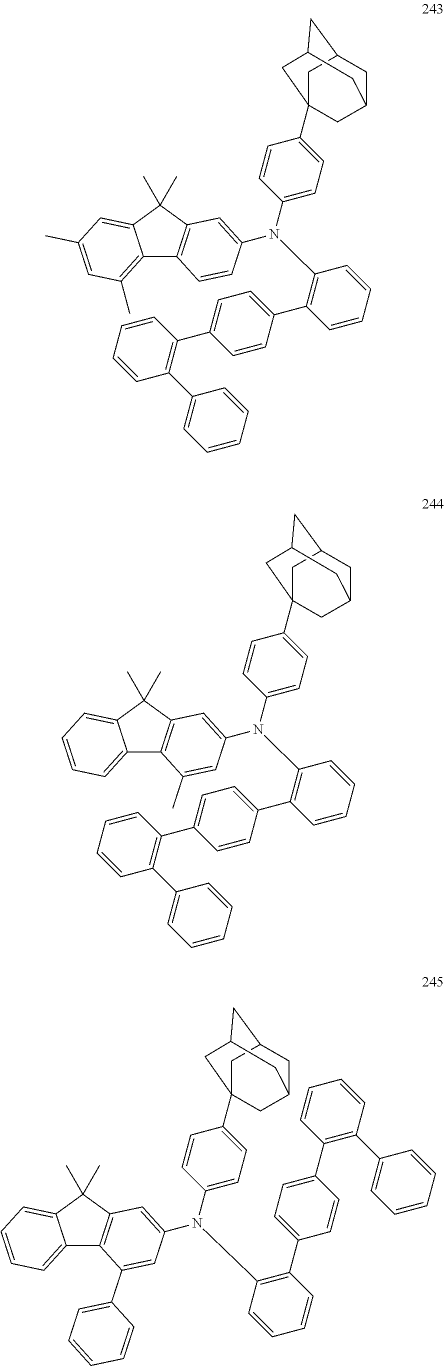 [SMILES: Cc1cc(C)c2c(c1)C(C)(C)c1cc(N(c3ccc(C45CC6CC(CC(C6)C4)C5)cc3)c3ccccc3-c3ccc(-c4ccccc4-c4ccccc4)cc3)ccc1-2 ; Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(C)C ; CC1(C)c2ccccc2-c2c(-c3ccccc3)cc(N(c3ccc(C45CC6CC(CC(C6)C4)C5)cc3)c3ccccc3-c3ccc(-c4ccccc4-c4ccccc4)cc3)cc21]

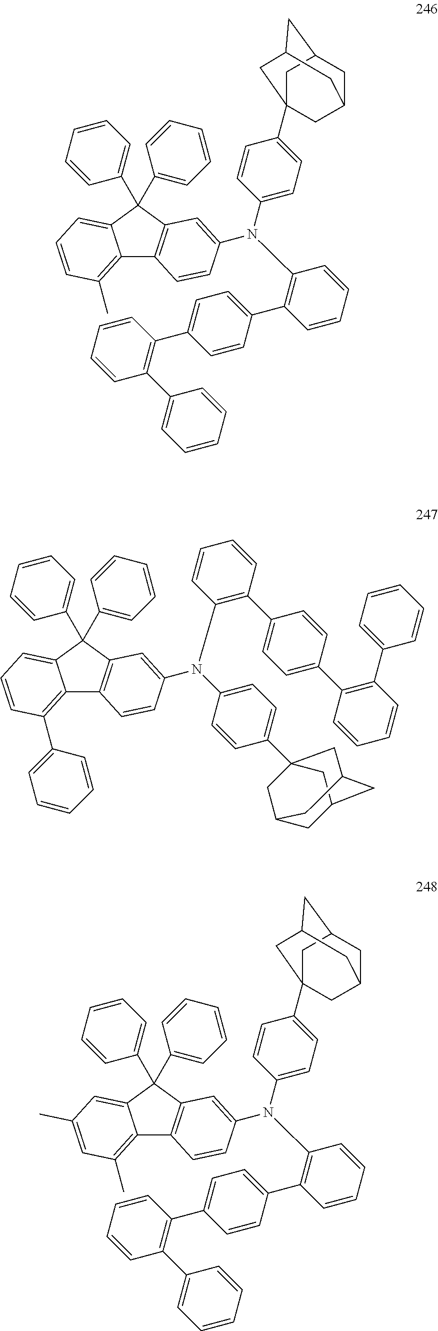 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C45CC6CC(CC(C6)C4)C5)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C45CC6CC(CC(C6)C4)C5)cc3)c3ccccc3-c3ccc(-c4ccccc4-c4ccccc4)cc3)ccc1-2]

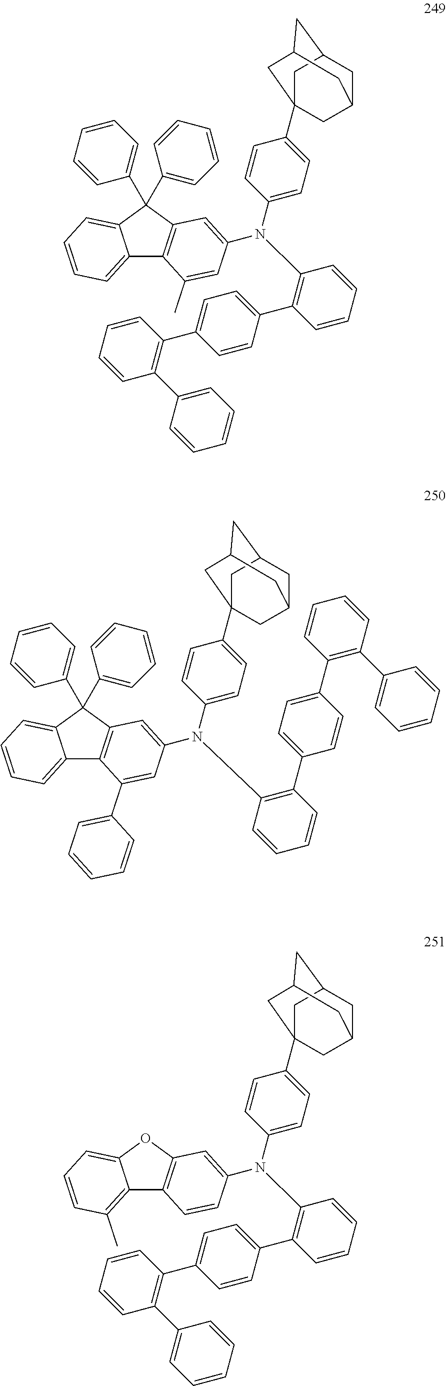 [SMILES: Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C45CC6CC(CC(C6)C4)C5)cc3)c3cc(-c4ccccc4)c4c(c3)C(c3ccccc3)(c3ccccc3)c3ccccc3-4)cc2)cc1 ; Cc1cccc2oc3cc(N(c4ccc(C56CC7CC(CC(C7)C5)C6)cc4)c4ccccc4-c4ccc(-c5ccccc5-c5ccccc5)cc4)ccc3c12]

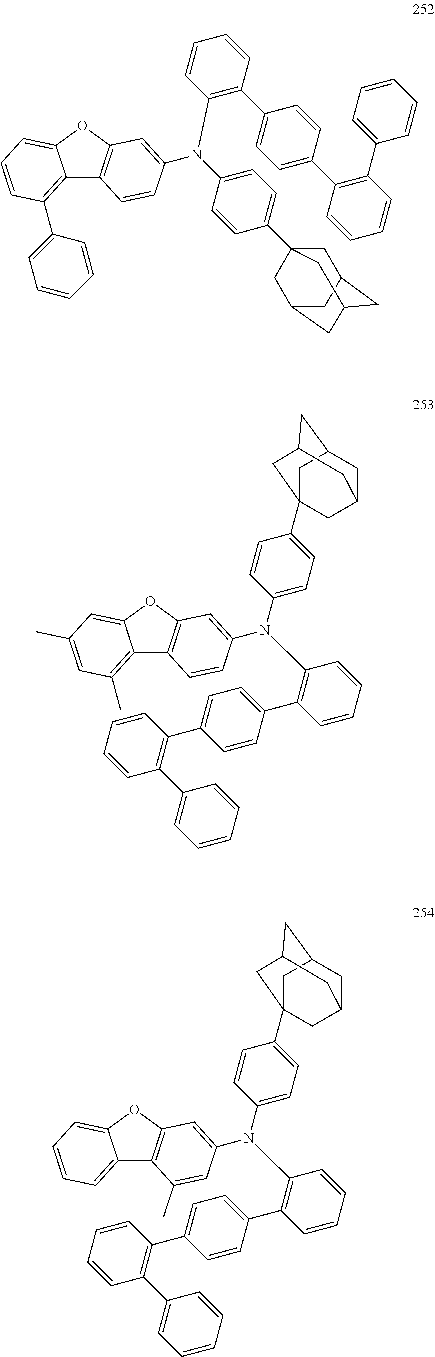 [SMILES: c1ccc(-c2ccccc2-c2ccc(-c3ccccc3N(c3ccc(C45CC6CC(CC(C6)C4)C5)cc3)c3ccc4c(c3)oc3cccc(-c5ccccc5)c34)cc2)cc1 ; Cc1cc(C)c2c(c1)oc1cc(N(c3ccc(C45CC6CC(CC(C6)C4)C5)cc3)c3ccccc3-c3ccc(-c4ccccc4-c4ccccc4)cc3)ccc12 ; Cc1cc(N(c2ccc(C34CC5CC(CC(C5)C3)C4)cc2)c2ccccc2-c2ccc(-c3ccccc3-c3ccccc3)cc2)cc2oc3ccccc3c12]

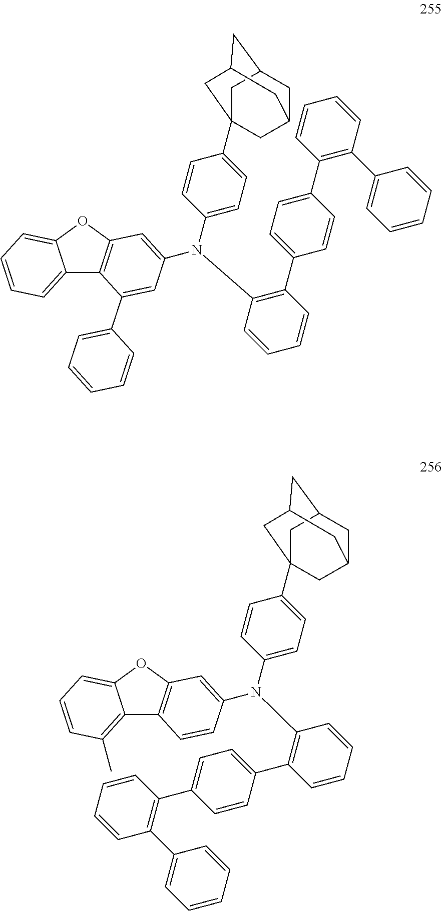 [SMILES: c1ccc(-c2ccccc2-c2ccc(-c3ccccc3N(c3ccc(C45CC6CC(CC(C6)C4)C5)cc3)c3cc(-c4ccccc4)c4c(c3)oc3ccccc34)cc2)cc1 ; Cc1cccc2oc3cc(N(c4ccc(C56CC7CC(CC(C7)C5)C6)cc4)c4ccccc4-c4ccc(-c5ccccc5-c5ccccc5)cc4)ccc3c12]

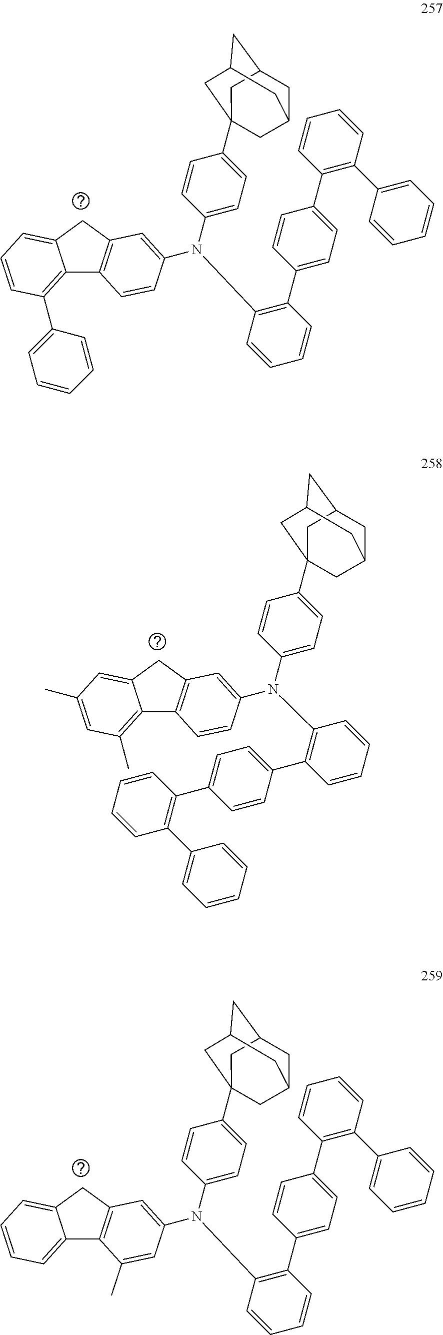 [SMILES: c1ccc(-c2ccccc2-c2ccc(-c3ccccc3N(c3ccc(C45CC6CC(CC(C6)C4)C5)cc3)c3ccc4c(c3)Cc3cccc(-c5ccccc5)c3-4)cc2)cc1 ; Cc1cc(C)c2c(c1)Cc1cc(N(c3ccc(C45CC6CC(CC(C6)C4)C5)cc3)c3ccccc3-c3ccc(-c4ccccc4-c4ccccc4)cc3)ccc1-2 ; Cc1cc(N(c2ccc(C34CC5CC(CC(C5)C3)C4)cc2)c2ccccc2-c2ccc(-c3ccccc3-c3ccccc3)cc2)cc2c1-c1ccccc1C2]

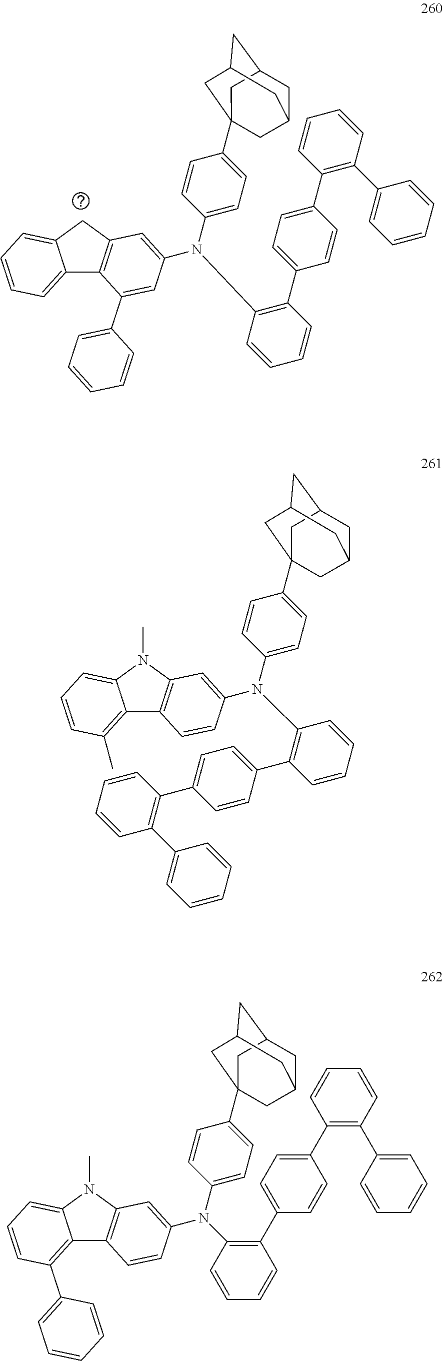 [SMILES: c1ccc(-c2ccccc2-c2ccc(-c3ccccc3N(c3ccc(C45CC6CC(CC(C6)C4)C5)cc3)c3cc4c(c(-c5ccccc5)c3)-c3ccccc3C4)cc2)cc1 ; Cc1cccc2c1c1ccc(N(c3ccc(C45CC6CC(CC(C6)C4)C5)cc3)c3ccccc3-c3ccc(-c4ccccc4-c4ccccc4)cc3)cc1n2C ; Cn1c2cc(N(c3ccc(C45CC6CC(CC(C6)C4)C5)cc3)c3ccccc3-c3ccc(-c4ccccc4-c4ccccc4)cc3)ccc2c2c(-c3ccccc3)cccc21]

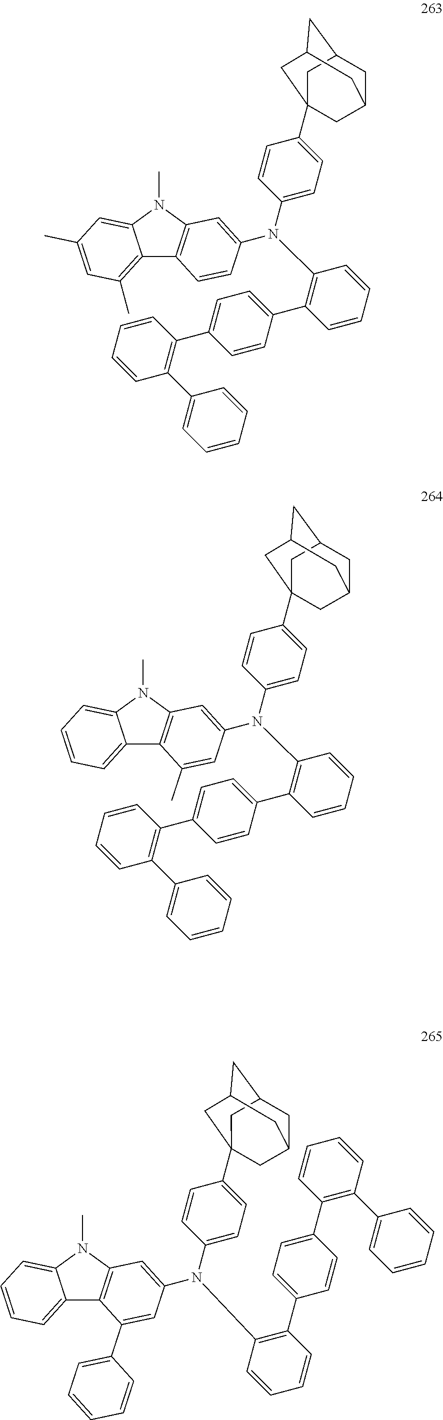 [SMILES: Cc1cc(C)c2c3ccc(N(c4ccc(C56CC7CC(CC(C7)C5)C6)cc4)c4ccccc4-c4ccc(-c5ccccc5-c5ccccc5)cc4)cc3n(C)c2c1 ; Cc1cc(N(c2ccc(C34CC5CC(CC(C5)C3)C4)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C45CC6CC(CC(C6)C4)C5)cc3)c3ccccc3-c3ccc(-c4ccccc4-c4ccccc4)cc3)cc21]

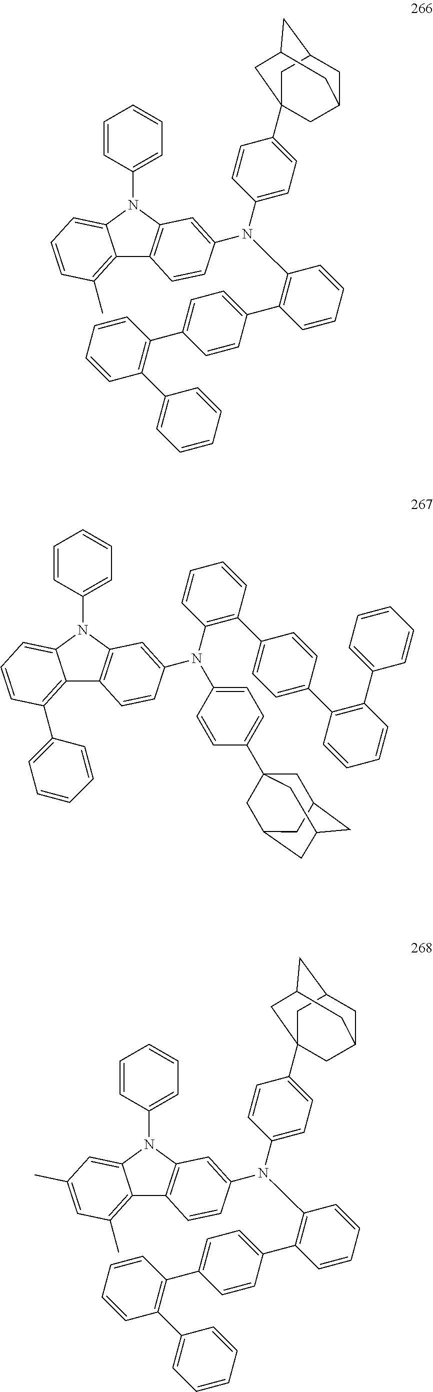 [SMILES: Cc1cccc2c1c1ccc(N(c3ccc(C45CC6CC(CC(C6)C4)C5)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C45CC6CC(CC(C6)C4)C5)cc3)c3ccc4c5c(-c6ccccc6)cccc5n(-c5ccccc5)c4c3)cc2)cc1 ; Cc1cc(C)c2c3ccc(N(c4ccc(C56CC7CC(CC(C7)C5)C6)cc4)c4ccccc4-c4ccc(-c5ccccc5-c5ccccc5)cc4)cc3n(-c3ccccc3)c2c1]

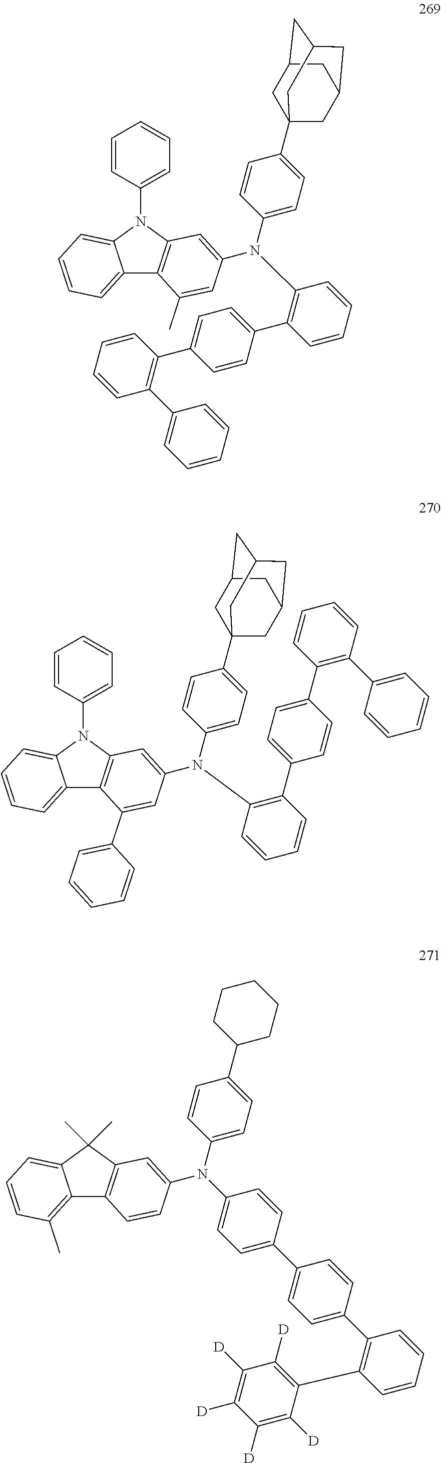 [SMILES: Cc1cc(N(c2ccc(C34CC5CC(CC(C5)C3)C4)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C45CC6CC(CC(C6)C4)C5)cc3)c3cc(-c4ccccc4)c4c5ccccc5n(-c5ccccc5)c4c3)cc2)cc1 ; [2H]c1c([2H])c([2H])c(-c2ccccc2-c2ccc(-c3ccc(N(c4ccc(C5CCCCC5)cc4)c4ccc5c(c4)C(C)(C)c4cccc(C)c4-5)cc3)cc2)c([2H])c1[2H]]

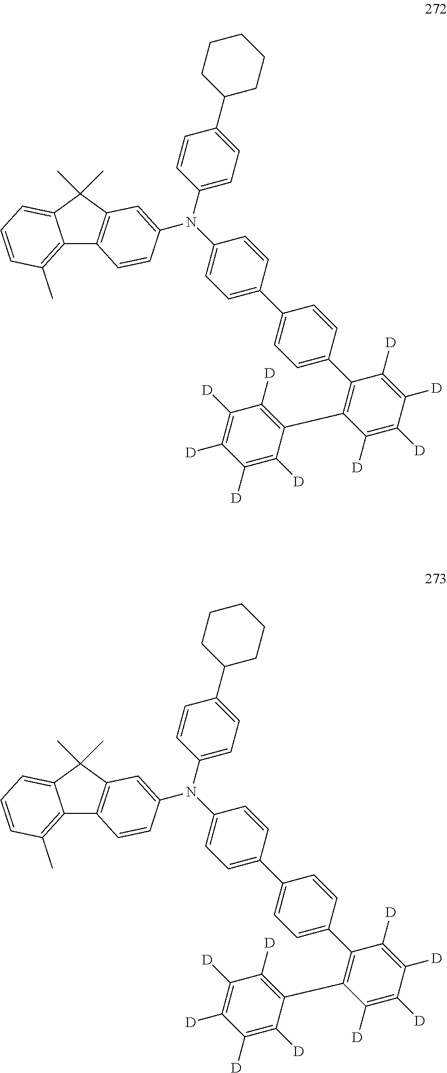 [SMILES: [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H] ; [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H]]

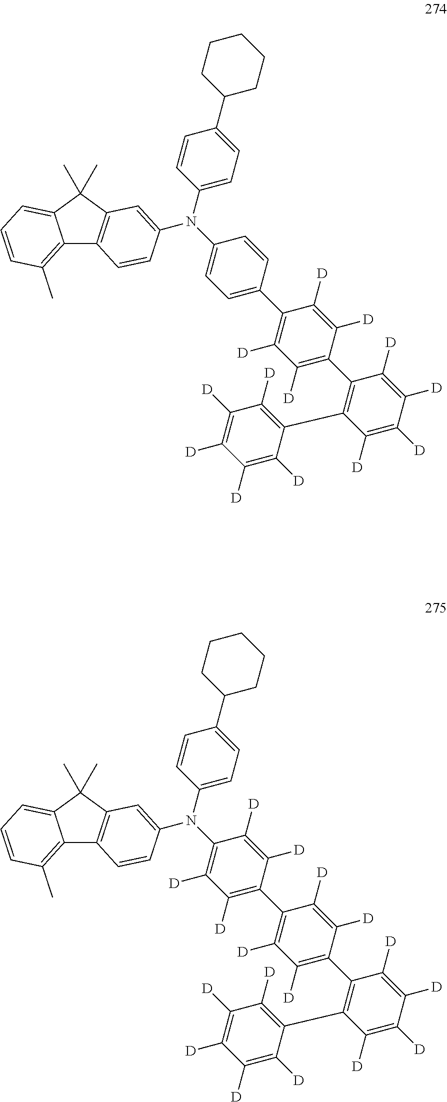 [SMILES: [2H]c1c([2H])c([2H])c(-c2c([2H])c([2H])c([2H])c([2H])c2-c2c([2H])c([2H])c(-c3ccc(N(c4ccc(C5CCCCC5)cc4)c4ccc5c(c4)C(C)(C)c4cccc(C)c4-5)cc3)c([2H])c2[2H])c([2H])c1[2H] ; [2H]c1c([2H])c([2H])c(-c2c([2H])c([2H])c([2H])c([2H])c2-c2c([2H])c([2H])c(-c3c([2H])c([2H])c(N(c4ccc(C5CCCCC5)cc4)c4ccc5c(c4)C(C)(C)c4cccc(C)c4-5)c([2H])c3[2H])c([2H])c2[2H])c([2H])c1[2H]]

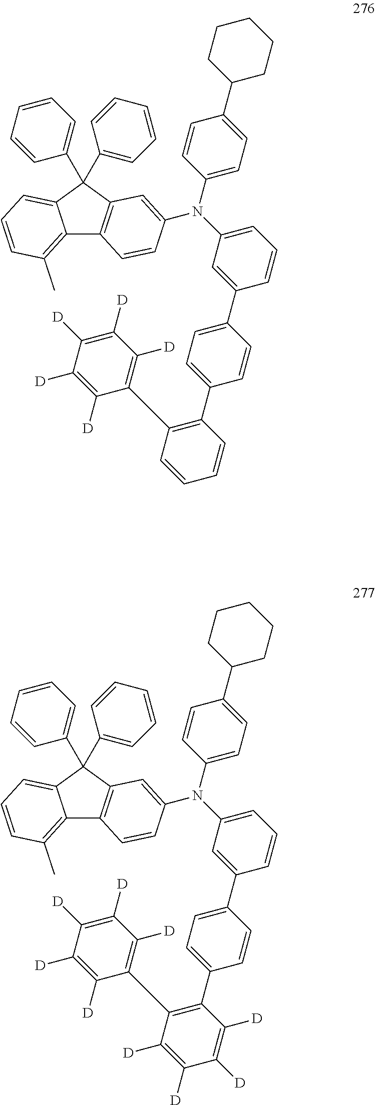 [SMILES: [2H]c1c([2H])c([2H])c(-c2ccccc2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H] ; [2H]c1c([2H])c([2H])c(-c2c([2H])c([2H])c([2H])c([2H])c2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H]]

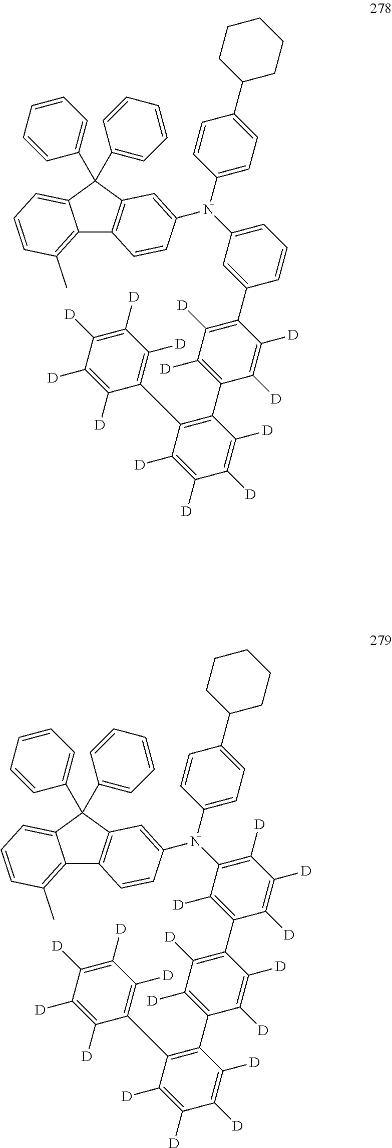 [SMILES: [2H]c1c([2H])c([2H])c(-c2c([2H])c([2H])c([2H])c([2H])c2-c2c([2H])c([2H])c(-c3cccc(N(c4ccc(C5CCCCC5)cc4)c4ccc5c(c4)C(c4ccccc4)(c4ccccc4)c4cccc(C)c4-5)c3)c([2H])c2[2H])c([2H])c1[2H] ; [2H]c1c([2H])c([2H])c(-c2c([2H])c([2H])c([2H])c([2H])c2-c2c([2H])c([2H])c(-c3c([2H])c([2H])c([2H])c(N(c4ccc(C5CCCCC5)cc4)c4ccc5c(c4)C(c4ccccc4)(c4ccccc4)c4cccc(C)c4-5)c3[2H])c([2H])c2[2H])c([2H])c1[2H]]

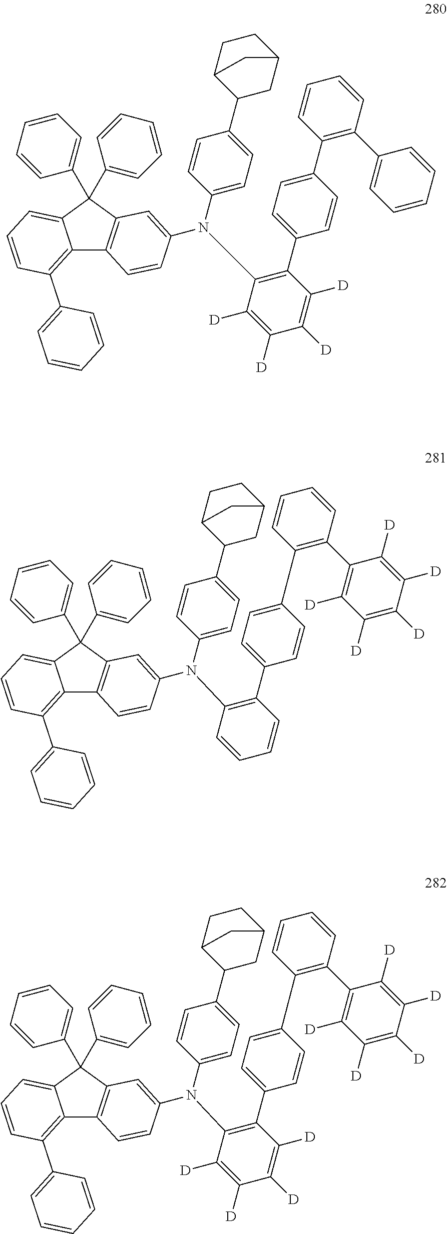 [SMILES: [2H]c1c([2H])c([2H])c(N(c2ccc(C3CC4CCC3C4)cc2)c2ccc3c(c2)C(c2ccccc2)(c2ccccc2)c2cccc(-c4ccccc4)c2-3)c(-c2ccc(-c3ccccc3-c3ccccc3)cc2)c1[2H] ; [2H]c1c([2H])c([2H])c(-c2ccccc2-c2ccc(-c3ccccc3N(c3ccc(C4CC5CCC4C5)cc3)c3ccc4c(c3)C(c3ccccc3)(c3ccccc3)c3cccc(-c5ccccc5)c3-4)cc2)c([2H])c1[2H] ; [2H]c1c([2H])c([2H])c(-c2ccccc2-c2ccc(-c3c([2H])c([2H])c([2H])c([2H])c3N(c3ccc(C4CC5CCC4C5)cc3)c3ccc4c(c3)C(c3ccccc3)(c3ccccc3)c3cccc(-c5ccccc5)c3-4)cc2)c([2H])c1[2H]]

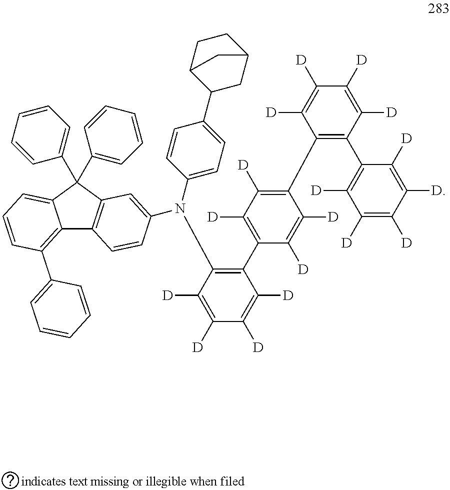

According to one or more embodiments of the present disclosure, a display device may include a base layer, a circuit layer disposed on the base layer, and a display element layer disposed on the circuit layer and including a light-emitting element, wherein the light-emitting element includes a first electrode, a second electrode disposed on the first electrode and a hole transport region disposed between the first electrode and the second electrode and containing the amine compound represented by Formula 1.

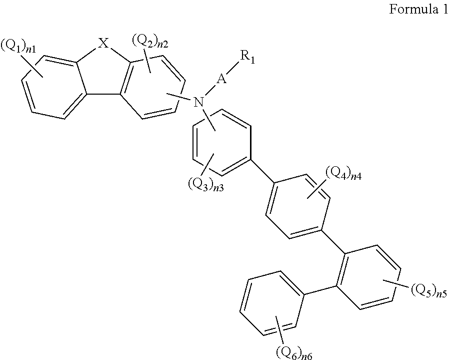

wherein, in Formula 1 above, n1, n2, n3, n4, and n5 may each independently be an integer of 0 to 4, n2 is an integer of 0 to 3, n6 is an integer of 0 to 5, n1 and n2 are not 0 at the same time, X is O, S, NR₂ of CR₃R₄, A is a substituted or unsubstituted arylene group having 6 to 30 ring-forming carbon atoms, R₁ is a substituted or unsubstituted cycloalkyl group having 3 to 10 ring-forming carbon atoms, R₂, R₃, R₄ may each independently be a hydrogen atom, a deuterium atom, a substituted or unsubstituted alkyl group having 1 to 30 carbon atoms, or a substituted or unsubstituted aryl group having 6 to 30 ring-forming carbon atoms, Q₁ and Q₂ may each independently be a substituted or unsubstituted alkyl group having 1 to 10 carbon atoms, or a substituted or unsubstituted aryl group having 6 to 30 ring-forming carbon atoms, and Q₃, Q₄, Q₅, and Q₆ may each independently be a hydrogen atom, a deuterium atom, a substituted or unsubstituted alkyl group having 1 to 10 carbon atoms, or a substituted or unsubstituted aryl group having 6 to 30 ring-forming carbon atoms.

According to one or more embodiments of the present disclosure, an amine compound is represented by Formula 1 above.

According to one or more embodiments, the amine compound represented by Formula 1 above may be represented by Formula 1-1 above.

According to one or more embodiments, the amine compound represented by Formula 1 above may be represented by any one selected from among Formulas 2-1 to 2-3 above.

According to one or more embodiments, the amine compound represented by Formula 1 above may be represented by any one selected from among Formulas 3-1 to 3-4 above.

In an embodiment, the amine compound represented by Formula 1 above may be represented by any one selected from among compounds in Compound Group 1.

### BRIEF DESCRIPTION OF THE DRAWINGS

The accompanying drawings are included to provide a further understanding of the present disclosure, and are incorporated in and constitute a part of this specification. The drawings illustrate embodiments of the present disclosure and, together with the description, serve to explain principles of the present disclosure. In the drawings:

FIG. 1 is a plan view of a display device according to an embodiment of the present disclosure;

FIG. 2 is a cross-sectional view of a display device according to an embodiment of the present disclosure;

FIG. 3 is a cross-sectional view schematically illustrating a light-emitting element according to an embodiment of the present disclosure;

FIG. 4 is a cross-sectional view schematically illustrating a light-emitting element according to an embodiment of the present disclosure;

FIG. 5 is a cross-sectional view schematically illustrating a light-emitting element according to an embodiment of the present disclosure;

FIG. 6 is a cross-sectional view schematically illustrating a light-emitting element according to an embodiment of the present disclosure;

FIGS. 7 and 8 are each a cross-sectional view of a display device according to an embodiment of the present disclosure;

FIG. 9 is a cross-sectional view illustrating a display device according to an embodiment of the present disclosure;

FIG. 10 is a cross-sectional view illustrating a display device according to an embodiment of the present disclosure; and

FIG. 11 is a view illustrating a vehicle in which a display device according to an embodiment of the present disclosure is disposed.

### DETAILED DESCRIPTION

The present disclosure may be modified in one or more suitable manners and have many forms, and thus specific embodiments will be illustrated in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements. In the accompanying drawings, the dimensions of each structure are exaggeratingly illustrated for clarity of the present disclosure. It will be understood that, although the terms “first,” “second,” etc., may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” and “the” are intended to include the plural forms as well, unless the context clearly indicates otherwise.

In the present application, it will be understood that the terms “include,” “have” and/or the like specify the presence of features, numbers, steps, operations, component, parts, or combinations thereof disclosed in the specification, but do not exclude the possibility of presence or addition of one or more other features, numbers, steps, operations, component, parts, or combinations thereof.

In the present application, when a layer, a film, a region, or a plate is referred to as being “on” or “in an upper portion of” another layer, film, region, or plate, it may be not only “directly on” the layer, film, region, or plate, but intervening layers, films, regions, or plates may also be present. On the contrary to this, when a layer, a film, a region, or a plate is referred to as being “below”, “in a lower portion of” another layer, film, region, or plate, it can be not only directly under the layer, film, region, or plate, but intervening layers, films, regions, or plates may also be present. In some embodiments, it will be understood that when a part is referred to as being “on” another part, it can be disposed above the other part, or disposed under the other part as well.

In the specification, the term “substituted or unsubstituted” may refer to substituted or unsubstituted with at least one substituent selected from among the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents illustrated above may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but the embodiment of the present disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but the embodiment of the present disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, etc., but the embodiment of the present disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. Although the number of carbon atoms is not specifically limited, it is 2 to 30, 2 to 20, or 2 to 10. Specific examples of the alkynyl group may include an ethynyl group, a propynyl group, etc., but are not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quarterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

In the specification, the fluorenyl group may be substituted, and two substituents may be bonded to each other to form a spiro structure. Examples of the substituted fluorenyl group are as follows. However, the embodiment of the present disclosure is not limited thereto.

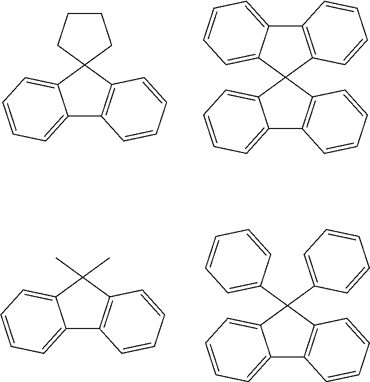

The heterocyclic group herein refers to any functional group or substituent derived from a ring containing at least one of B, O, N, P, Si, or S as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heterocyclic group may contain at least one of B, O, N, P, Si or S as a heteroatom. When the heterocyclic group contains two or more heteroatoms, the two or more heteroatoms may be the same as or different from each other. The heterocyclic group may be a monocyclic heterocyclic group or a polycyclic heterocyclic group, and includes a heteroaryl group. The number of ring-forming carbon atoms in the heterocyclic group may be 2 to 30, 2 to 20, or 2 to 10.

In the specification, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but the embodiment of the present disclosure is not limited thereto.

In the specification, the heteroaryl group may contain at least one of B, O, N, P, Si, or S as a heteroatom. When the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the specification, the above description of the aryl group may be applied to an arylene group except that the arylene group is a divalent group. The above description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but the embodiment of the present disclosure is not limited thereto.

In the specification, the number of ring-forming carbon atoms in the carbonyl group is not specifically limited, but may be 1 to 40, 1 to 30, or 1 to 20. For example, the carbonyl group may have the following structures, but the embodiment of the present disclosure is not limited thereto.

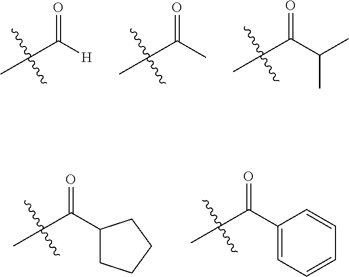

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the present disclosure is not limited thereto.

In the specification, an oxy group may refer to that an oxygen atom is bonded to the alkyl group or the aryl group as defined above. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but the embodiment of the present disclosure is not limited thereto.

The boron group herein may refer to that a boron atom is bonded to the alkyl group or the aryl group as defined above. The boron group includes an alkyl boron group and an aryl boron group. Examples of the boron group may include a dimethylboron group, a trimethylboron group, a t-butyldimethylboron group, a diphenylboron group, a phenylboron group, etc., but the embodiment of the present disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, etc., but the embodiment of the present disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described above.

In the specification, the aryl group among an aryloxy group, an arylthio group, an arylsulfoxy group, an arylamino group, an arylboron group, an arylsilyl group, an arylamine group is the same as the examples of the aryl group described above.

In the specification, a direct linkage may refer to a single bond.

In some embodiments, in the specification,

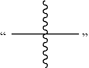

and “-*” refer to a position to be connected.

Hereinafter, embodiments of the present disclosure will be described with reference to the accompanying drawings.

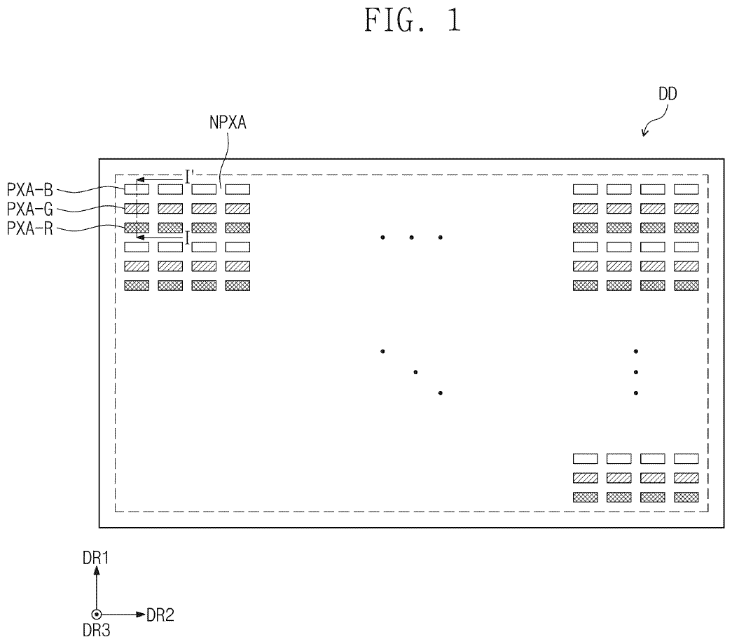

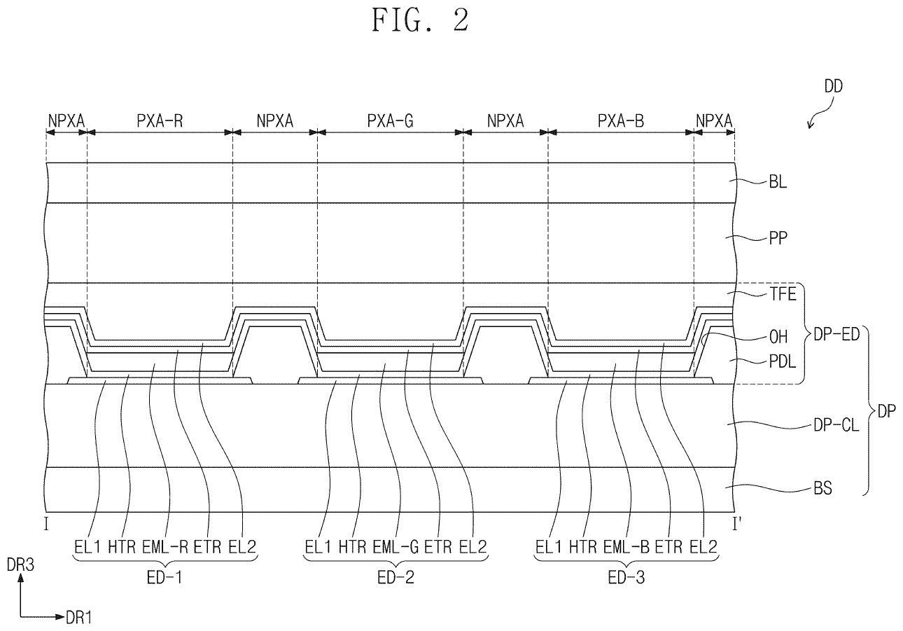

FIG. 1 is a plan view illustrating an embodiment of a display apparatus DD. FIG. 2 is a cross-sectional view of the display apparatus DD of the embodiment. FIG. 2 is a cross-sectional view illustrating a part taken along line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include a plurality of light emitting devices ED-1, ED-2, and ED-3. The optical layer PP may be disposed on the display panel DP to control reflected light in the display panel DP due to external light. The optical layer PP may include, for example, a polarization layer or a color filter layer. In some embodiments, unlike the configuration illustrated in the drawing, the optical layer PP may not be provided from the display apparatus DD of an embodiment.

A base substrate BL may be disposed on the optical layer PP. The base substrate BL may be a member which provides a base surface on which the optical layer PP disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, unlike the configuration illustrated, in an embodiment, the base substrate BL may not be provided.

The display apparatus DD according to an embodiment may further include a filling layer. The filling layer may be disposed between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting devices ED-1, ED-2, and ED-3 disposed between portions of the pixel defining film PDL, and an encapsulation layer TFE disposed on the light emitting devices ED-1, ED-2, and ED-3.

The base layer BS may be a member which provides a base surface on which the display device layer DP-ED is disposed. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is disposed on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting devices ED-1, ED-2, and ED-3 of the display device layer DP-ED.

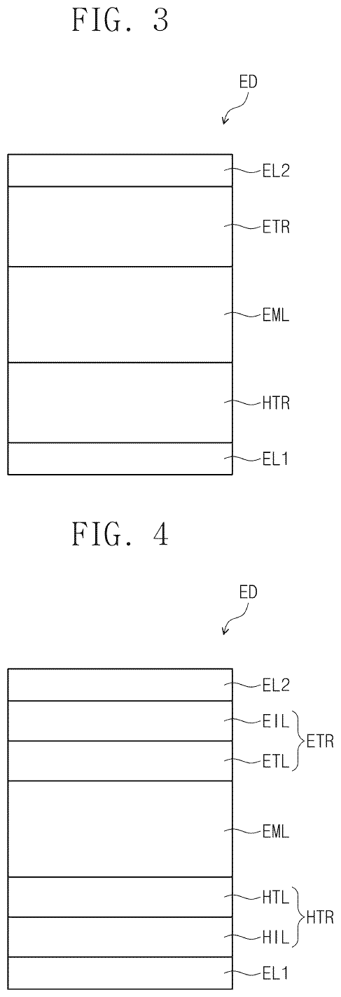

Each of the light emitting devices ED-1, ED-2, and ED-3 may have a structure of each light emitting device ED of embodiments according to FIGS. 3 to 6, which will be described later. Each of the light emitting devices ED-1, ED-2, and ED-3 may include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting devices ED-1, ED-2, and ED-3 are disposed in openings OH defined in the pixel defining film PDL, and the hole transport region HTR, the electron transport region ETR, and the second electrode EL2 are provided as a common layer in the entire light emitting devices ED-1, ED-2, and ED-3. However, the embodiment of the present disclosure is not limited thereto, and unlike the configuration illustrated in FIG. 2, the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting devices ED-1, ED-2, and ED-3 in an embodiment may be provided by being patterned in an inkjet printing method.

The encapsulation layer TFE may cover the light emitting devices ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not particularly limited thereto. The encapsulation-organic film may include an acrylic-based compound, an epoxy-based compound, and/or the like. The encapsulation-organic film may include a photopolymerizable organic material, but the embodiment of the present disclosure is not particularly limited thereto.

The encapsulation layer TFE may be disposed on the second electrode EL2 and may be disposed filling the opening OH.

Referring to FIGS. 1 and 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light emitting devices ED-1, ED-2, and ED-3 is emitted. The light emitting regions PXA-R, PXA-G, and PXA-B may be spaced apart from each other on a plane.

Each of the light emitting regions PXA-R, PXA-G, and PXA-B may be a region divided by the pixel defining film PDL. The non-light emitting areas NPXA may be areas between the adjacent light emitting areas PXA-R, PXA-G, and PXA-B, which correspond to the pixel defining film PDL. In some embodiments, in the specification, the light emitting regions PXA-R, PXA-G, and PXA-B may respectively correspond to pixels. The pixel defining film PDL may divide the light emitting devices ED-1, ED-2, and ED-3. The emission layers EML-R, EML-G, and EML-B of the light emitting devices ED-1, ED-2, and ED-3 may be disposed in openings OH defined in the pixel defining film PDL and separated from each other.

The light emitting regions PXA-R, PXA-G, and PXA-B may be divided into a plurality of groups according to the color of light generated from the light emitting devices ED-1, ED-2, and ED-3. In the display apparatus DD of an embodiment illustrated in FIGS. 1 and 2, three light emitting regions PXA-R, PXA-G, and PXA-B, which emit red light, green light, and blue light, respectively, are illustrated as examples. For example, the display device DD of an embodiment may include the red light emitting region PXA-R, the green light emitting region PXA-G, and the blue light emitting region PXA-B that are separated from each other.

In the display apparatus DD according to an embodiment, the plurality of light emitting devices ED-1, ED-2 and ED-3 may be configured to emit light beams having wavelengths different from each other. For example, in an embodiment, the display apparatus DD may include a first light emitting device ED-1 that is configured to emit red light, a second light emitting device ED-2 that is configured to emit green light, and a third light emitting device ED-3 that is configured to emit blue light. For example, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, the embodiment of the present disclosure is not limited thereto, and the first to third light emitting devices ED-1, ED-2, and ED-3 may be configured to emit light beams in substantially the same wavelength range or at least one light emitting device may be configured to emit a light beam in a wavelength range different from the others. For example, the first to third light emitting devices ED-1, ED-2, and ED-3 may all emit blue light.

The light emitting regions PXA-R, PXA-G, and PXA-B in the display apparatus DD according to an embodiment may be arranged in a stripe form. Referring to FIG. 1, the plurality of red light emitting regions PXA-R, the plurality of green light emitting regions PXA-G, and the plurality of blue light emitting regions PXA-B each may be arranged along a second directional axis DR2. In some embodiments, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but the embodiment of the present disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas when viewed on a plane defined by the first directional axis DR1 and the second directional axis DR2 (e.g., in a plan view).

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display apparatus DD. For example, the arrangement form of the light emitting regions PXA-R, PXA-G, and PXA-B may be a pentile (PENTILE™) arrangement form or a diamond (Diamond Pixel™) arrangement form.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

Hereinafter, FIG. 3 to FIG. 6 are cross-sectional views schematically showing light emitting devices according to embodiments. The light emitting device ED of an embodiment may include a first electrode EL1, a hole transport region HTR, an emission layer EML, an electron transport region ETR, and a second electrode EL2 stacked in order.

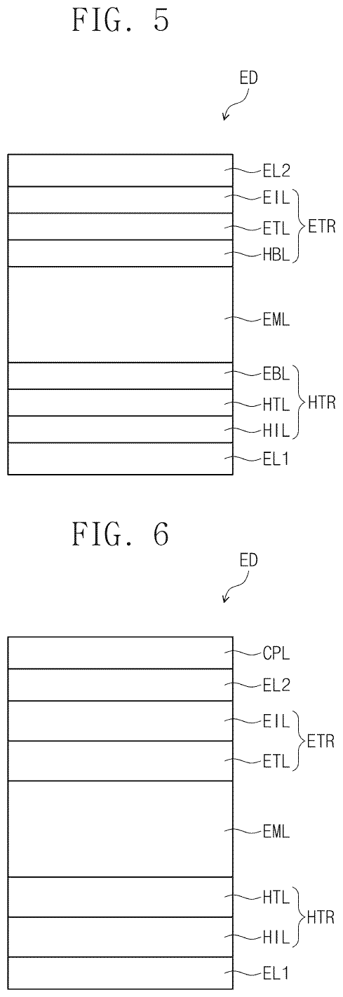

Compared with FIG. 3, FIG. 4 illustrates a cross-sectional view of a light emitting device ED of an embodiment, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light emitting device ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting device ED of an embodiment including a capping layer CPL disposed on a second electrode EL2.

The light-emitting element ED according to an embodiment may contain an amine compound according to an embodiment which will be described in more detail later, in at least one functional layer, such as a hole transport region HTR, a light-emitting layer EML, and an electron transport region ETR.

The first electrode EL1 has conductivity (e.g., is a conductor). The first electrode EL1 may be formed of a metal material, a metal alloy, or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, or an oxide thereof.

When the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but the embodiment of the present disclosure is not limited thereto. In some embodiments, the embodiment of the present disclosure is not limited thereto, and the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one selected from among a hole injection layer HIL, a hole transport layer HTL, a buffer layer or a light-emitting auxiliary layer, and an electron-blocking layer EBL. The hole transport region HTR may have a thickness of, for example, about 50 Å to about 15,000 Å.

The hole transport region HTR may have a single layer formed of a single material, a single layer formed of a plurality of different materials, or a multi-layer structure having multiple layers formed of a plurality of different materials.

For example, the hole transport region HTR may have a single-layer structure of the hole injection layer HIL or the hole transport layer HTL or have a single-layer structure formed of a hole injection material and a hole transport material. In some embodiments, the hole transport region HTR may have a single-layer structure formed of a plurality of different materials or have a structure in which layers are sequentially stacked from the first electrode EL1, such as a structure of a hole injection layer HIL/hole transport layer HTL, a hole injection layer HIL/hole transport layer HTL/buffer layer, a hole injection layer HIL/buffer layer, a hole transport layer HTL/buffer layer, or hole injection layer HIL/hole transport layer HTL/electron-blocking layer EBL. However, an embodiment of the present disclosure is not limited thereto.

When the hole transport region HTR includes a hole transport layer HTL, the hole transport layer HTL may have a single layer formed of a single material, a single layer formed of a plurality of different materials, or a multi-layer structure having multiple layers formed of a plurality of different materials. When the hole transport region HTR has multiple layers, the multiple layers each have a different refractive index.

The hole transport region HTR may be formed by utilizing one or more suitable methods such as a vacuum deposition method, a spin coating method, a casting method, the Langmuir-Blodgett (LB) method, an inkjet printing method, a laser printing method, and a laser induced thermal imaging (LITI) method.

In the light-emitting element ED according to an embodiment, the hole transport region HTR may contain an amine compound according to an embodiment. In the light-emitting element ED according to an embodiment, at least any one selected from among the hole injection layer HIL, the hole transport layer HTL, and the electron-blocking layer EBL may contain an amine compound represented by Formula 1.

The amine compound according to an embodiment has a structure in which a first substituent, a second substituent, and a third substituent are bonded to a core nitrogen atom, wherein the first substituent includes a dibenzofuranyl moiety, a dibenzothiophenyl moiety, a carbazole moiety, or a fluorenyl moiety. The second substituent has a structure in which a cyclohexyl group, a bicycloheptanyl group, or an aliphatic cycloalkyl group of an adamantyl group, is bonded via an arylene linker. The third substituent has a substituted or unsubstituted quarterphenyl group structure. In the third substituent, a benzene ring of the quarterphenyl group that is bonded to a nitrogen atom is named as a first benzene ring, and benzene rings are sequentially named as second, third, and fourth benzene rings from the benzene ring connected to the first benzene ring. In this case, the third benzene ring is bonded at a para position with respect to the carbon atom among ring-forming carbon atoms of the second benzene ring, which forms a bond between the first benzene ring and the second benzene ring. In some embodiments, the fourth benzene ring is bonded at an ortho position, with respect to the carbon atom among ring-forming carbon atoms of the third benzene ring, which forms a bond with the second benzene ring. The amine compound according to an embodiment having such a structure exhibits high thermal properties and thus improves element lifespan and efficiency when applied to the light-emitting element ED according to an embodiment. In some embodiments, due to an excellent or suitable charge transport ability, a balance between holes and electrons may be achieved in the light-emitting layer EML. In some embodiments, the amine compound according to an embodiment has low refractive properties, and thus a ratio of propagating light in a downward direction may be reduced when applied to the light-emitting element ED. Therefore, an amount of internally absorbed light may be reduced, and an amount of externally emitted light may increase. As a result, the luminous efficiency of the light-emitting device ED may be improved.

The light-emitting element ED according to an embodiment contains an amine compound according to an embodiment, which will be described in more detail later. Because the light-emitting element ED according to an embodiment contains the amine compound according to an embodiment, a charge transfer may be prompted, and thus luminous efficiency characteristics may be improved.

The amine compound according to an embodiment is represented by Formula 1.

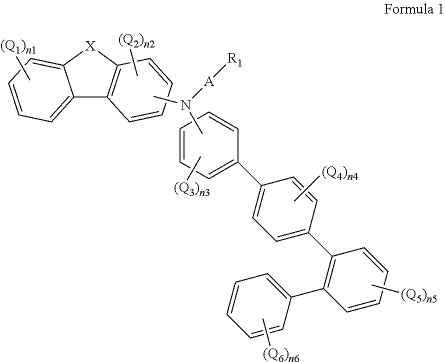

In Formula 1, A is a substituted or unsubstituted arylene group having 6 to 30 ring-forming carbon atoms. For example, A may be an unsubstituted phenylene group.

In Formula 1, R₁ is a substituted or unsubstituted cycloalkyl group having 3 to 10 ring-forming carbon atoms. For example, R₁ may be an unsubstituted cyclohexyl group, an unsubstituted bicycloheptanyl group, or an unsubstituted adamantly group.

In Formula 1, X may be O, S, NR₂, or CR₃R₄.

In Formula 1, R₂, R₃, and R₄ each are independently a hydrogen atom, a substituted or unsubstituted alkyl group having 1 to 30 carbon atoms, or a substituted or unsubstituted aryl group having 6 to 30 ring-forming carbon atoms. For example, R₂, R₃, and R₄ may each independently be a hydrogen atom, an unsubstituted methyl group, or an unsubstituted phenyl group.

In Formula 1, Q₁ and Q₂ may each independently be a substituted or unsubstituted alkyl group having 1 to 10 carbon atoms, or a substituted or unsubstituted aryl group having 6 to 30 ring-forming carbon atoms. For example, Q₁ and Q₂ may each independently be substituted or unsubstituted methyl group, or a substituted or unsubstituted phenyl group.

In Formula 1, Q₃, Q₄, Q₅ and Q₆ may each independently be a hydrogen atom, a deuterium atom, a substituted or unsubstituted alkyl group having 1 to 10 carbon atoms, or a substituted or unsubstituted aryl group having 6 to 30 ring-forming carbon atoms. For example, Q₃, Q₄, Q₅, and Q₆ may each independently be a hydrogen atom, or a deuterium atom.

In Formula 1, n1, n3, n4, and n5 may each independently be an integer of 0 to 4.

In Formula 1, when n1 is 0, the amine compound according to an embodiment may be unsubstituted with Q₁ and be substituted with four hydrogen atoms. When n1 is an integer of 2 or more, Q₁s that are provided in plurality may be all the same, or at least one selected from among the plurality of Q₁s may be different.

In Formula 1, when n3 is 0, the amine compound according to an embodiment may be unsubstituted with Q₃ and be substituted with four hydrogen atoms. A case where n3 is 4, and all Q₃s are hydrogen atoms may be the same as a case where n3 is 0. When n3 is an integer of 2 or more, Q₃s provided in plurality may be all the same, or at least one selected from among the plurality of Q₅s may be different.

In Formula 1, when n4 is 0, the amine compound according to an embodiment may be unsubstituted with Q₄ and be substituted with four hydrogen atoms. A case where n4 is 4, and all Q₄s are hydrogen atoms may be the same as a case where n4 is 0. When n4 is an integer of 2 or more, Q₄s provided in plurality may be all the same, or at least one selected from among the plurality of Q₄s may be different.

In Formula 1, when n5 is 0, the amine compound according to an embodiment may be unsubstituted with Q₅ and be substituted with four hydrogen atoms. A case where n5 is 4, and all Q₅s are hydrogen atoms may be the same as a case where n5 is 0. When n5 is an integer of 2 or more, Q₅s provided in plurality may be all the same, or at least one selected from among the plurality of Q₅s may be different.

In Formula 1, n2 is an integer of 0 to 3.

In Formula 1, when n2 is 0, the amine compound according to an embodiment may be unsubstituted with Q₂ and be substituted with three hydrogen atoms. When n2 is an integer of 2 or more, Q₂s that is provided in plurality may be all the same, or at least one selected from among the plurality of Q₂s may be different.

In Formula 1, n1 and n2 are not 0 concurrently (e.g., simultaneously). For example, when n1 is 0, n2 is an integer of 1 to 3. When n2 is 0, n1 is an integer of 1 to 4.

In Formula 1, n6 is an integer of 0 to 5.

In Formula 1, when n6 is 0, the amine compound according to an embodiment may be unsubstituted with Q₆ and be substituted with five hydrogen atoms. A case where n6 is 5, and all Q₆ are hydrogen atoms may be the same as a case where n6 is 0. When n6 is an integer of 2 or more, Q₆ that is provided in plurality may be all the same, or at least one selected from among the plurality of Q₆ may be different.

In an embodiment, the amine compound represented by Formula 1 may be represented by Formula 1-1.

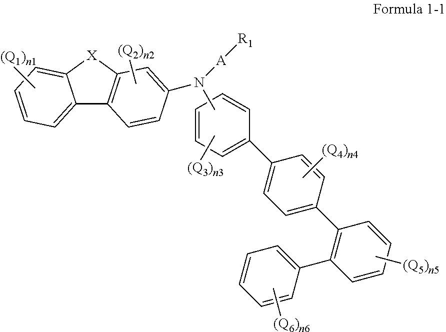

Formula 1-1 represents a case where the position of carbon is specified in the structure of Formula 1 in which a substituent structure corresponding to a fluorenyl moiety, a carbazole moiety, a dibenzofuranyl moiety, or a dibenzothiophenyl moiety is bonded to a nitrogen atom. For example, Formula 1-1 represents a case where, in the structure of Formula 1, the position of carbon is specified as a structure in which the substituent structure is bonded to the nitrogen atom at a meta position with respect to X position. When the substituent structure is bonded to the nitrogen atom at the meta position with respect to the X position, it is possible to have an energy level of HOMO having excellent or suitable charge-transporting properties, and thus luminous efficiency may be further improved.

In some embodiments, in Formula 1-1, the same descriptions as in Formula 1 above may be applied to n1 to n6, X, A, R₁ to R₄, and Q₁ to Q₆.

In an embodiment, the amine compound represented by Formula 1 may be represented by any one selected from among Formulas 2-1 to 2-3.

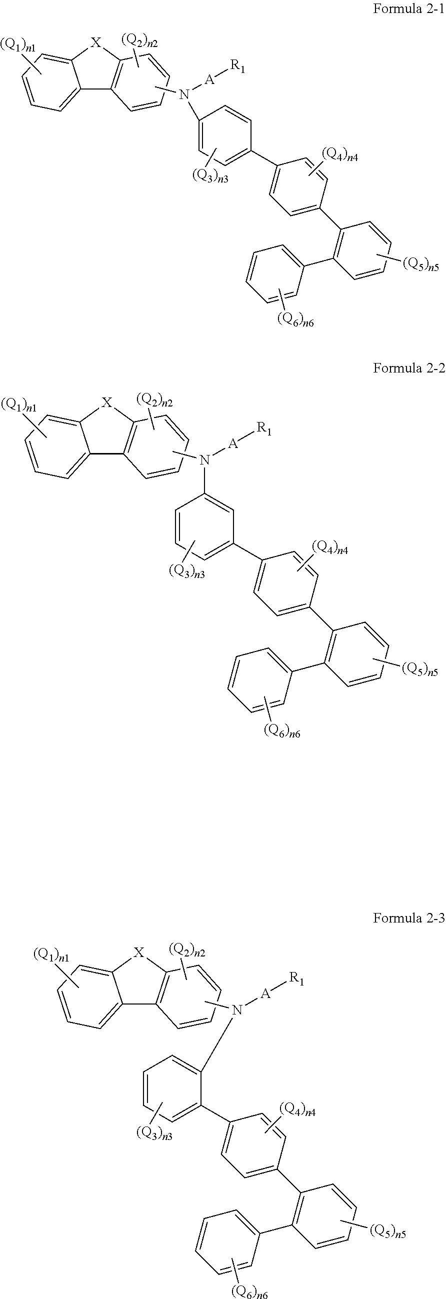

Formulas 2-1 to 2-3 represent cases where the position of carbon is specified in the structure of Formula 1 in which the quarterphenyl moiety included in the amine compound according to an embodiment is bonded to a nitrogen atom. A benzene ring of the quarterphenyl moiety bonded to the nitrogen atom is named as a first benzene ring, and benzene rings are sequentially named as second, third, and fourth benzene rings from the benzene ring connected to the first benzene ring. Formula 2-1 represents a case where a nitrogen atom is bonded at a para-position with respect to the second benzene ring, which is connected to the first benzene ring. Formula 2-2 represents a case where a nitrogen atom is bonded at a meta-position with respect to the second benzene ring, which is connected to the first benzene ring. Formula 2-3 represents a case where a nitrogen atom is bonded at an ortho position with respect to the second benzene ring, which is connected to the first benzene ring.

In Formulas 2-1 to 2-3, the same descriptions as in Formula 1 above may be applied to n1 to n6, X, A, R₁ to R₄ and Q₁ to Q₆.

In an embodiment, the amine compound represented by Formula 1 may be represented by any one selected from among Formulas 3-1 to 3-4.

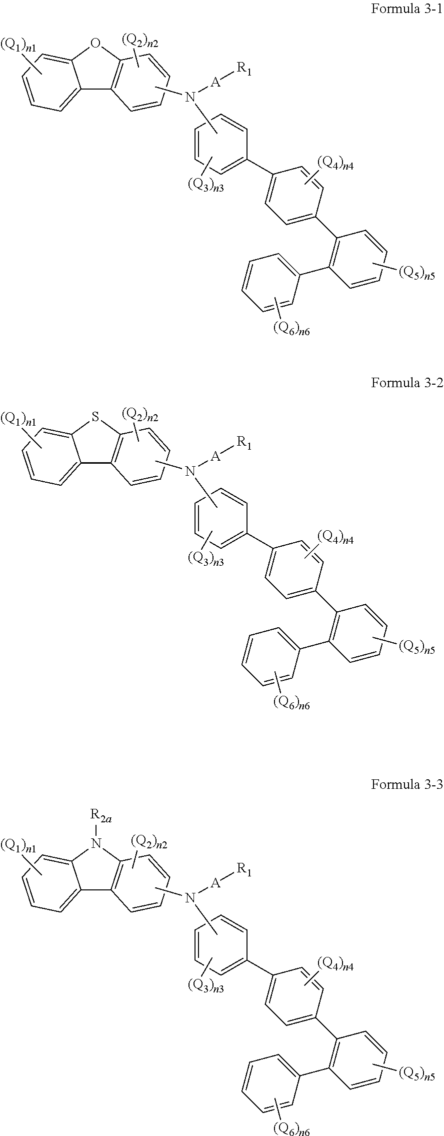

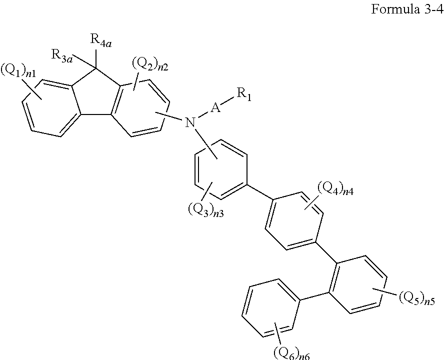

Formulas 3-1 to 3-4 represent cases where X is specified in the structure of Formula 1 and thus the amine compounds according to an embodiment are specified to contain a dibenzofuranyl moiety, a dibenzothiophennyl moiety, a carbozole moiety, and a fluorenyl moiety, respectively. Formula 3-1 represents a case where the amine compound represented by Formula 1 is specified to contain a dibenzofuranyl moiety, Formula 3-2 represents a case where the amine compound represented by Formula 1 is specified to contain a dibenzothiophenyl moiety, Formula 3-3 represents a case where the amine compound represented by Formula 1 is specified to contain a carbazole moiety, and Formula 3-4 represents a case where the amine compound represented by Formula 1 is specified to contain a fluorenyl moiety.

In Formulas 3-3 and 3-4, R₂ₐ, R₃ₐ, and R_{4b} may each independently be a hydrogen atom, an unsubstituted alkyl group having 1 to 30 carbon atoms, or an unsubstituted aryl group having 6 to 30 ring-forming carbon atoms. For example, R₂ₐ, R₃ₐ, and R₄ₐ may each independently be an unsubstituted methyl group, or an unsubstituted phenyl group.

In Formulas 3-1 to 3-4, the same descriptions as in Formula 1 above may be applied to n1 to n6, A, R₁, and Q₁ to Q₆.

In an embodiment, the amine compound represented by Formula 1 may be represented by Formula 4.

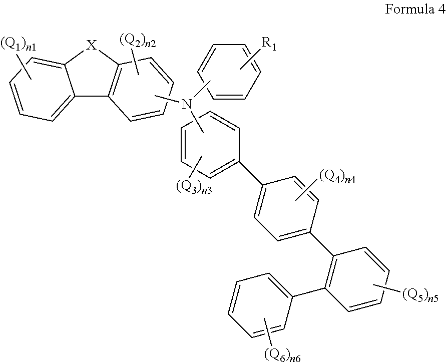

Formula 4 represents a case where A is specified as an unsubstituted phenylene group in the structure of Formula 1.

In Formula 4, the same descriptions as in Formula 1 above may be applied to n1 to n6, X, R₁ to R₄, and Q₁ to Q₆.

In an embodiment, the amine compound represented by Formula 4 may be represented by Formula 4-1.

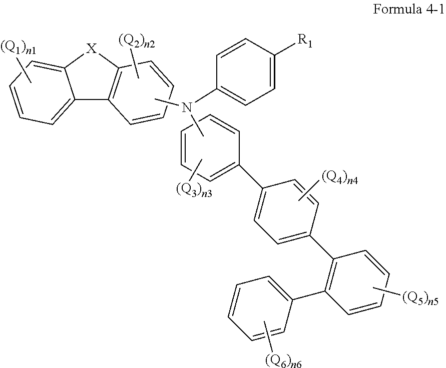

Formula 4-1 represents a case where, in the structure of Formula 4, a position at which R₁ is substituted with an unsubstituted phenylene group is specified. Formula 4-1 represents a case where R₁ is substituted at a para position with respect to the position at which the nitrogen atom is substituted.

In Formula 4-1, the same descriptions as in Formula 1 above may be applied to n1 to n6, X, R₁ to R₄, and Q₁ to Q₆.

In an embodiment, the amine compound represented by Formula 1 may be represented by Formula 5.

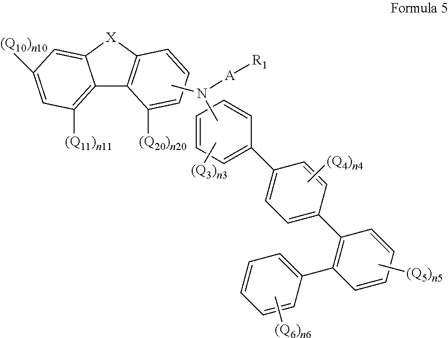

Formula 5 represents a case where substitution positions of Q₁₀, Q₁₁, and Q₁₂, that may be substituted with a dibenzofuranyl moiety, a dibenzothiophenyl moiety, a carbazole moiety, or a fluorenyl moiety are specified in Formula 1. In the benzene moiety that is bonded in a counterclockwise direction with respect to X, Q₁₀ may be bonded at a meta position with respect to carbon bonded to X. In the benzene moiety bonded in a counterclockwise direction with respect to X, Q₁₁ may be bonded at another meta position with respect to carbon bonded to X. In the benzene moiety bonded in a clockwise direction with respect to X, Q₂₀ may be bonded at a meta position with respect to carbon bonded to X.

In Formula 5, Q₁₀ may be an unsubstituted alkyl group having 1 to 10 carbon atoms. For example, Q₁₀ may be an unsubstituted methyl group.

In Formula 5, Q₁₁ and Q₁₂ may each independently be an unsubstituted alkyl group having 1 to 10 carbon atoms or an unsubstituted aryl group having 6 to 30 ring-forming carbon atoms. For example, Q₁₁ and Q₁₂ may each independently be an unsubstituted methyl group, or an unsubstituted phenyl group.

In Formula 5, n10, n11, and n20 may each independently be 0 or 1.

In Formula 5, when n10 is 0, the amine compound according to an embodiment may be unsubstituted with Q₁₀ and be substituted with a hydrogen atom. In Formula 5, when n11 is 0, the amine compound according to an embodiment may be unsubstituted with Q₁₁ and be substituted with a hydrogen atom. In Formula 5, when n20 is 0, the amine compound according to an embodiment may be unsubstituted with Q₂₀ and be substituted with a hydrogen atom. n10, n11, and n20 are not 0 at the same time. For example, when n10 and n11 are 0, n20 is 1; when n10 and n20 are 0, n11 is 1; and when n11 and n20 are 0, n10 is 1.

In Formula 5, the same descriptions as in Formula 1 above may be applied to n3 to n6, X, A, R₁ to R₄, and Q₃ to Q₆.

In an embodiment, the amine compound represented by Formula 1 may be represented by any one selected from among compounds in Compound Group 1. The hole transport region HTR may include at least one selected from among compounds presented in Compound Group 1.

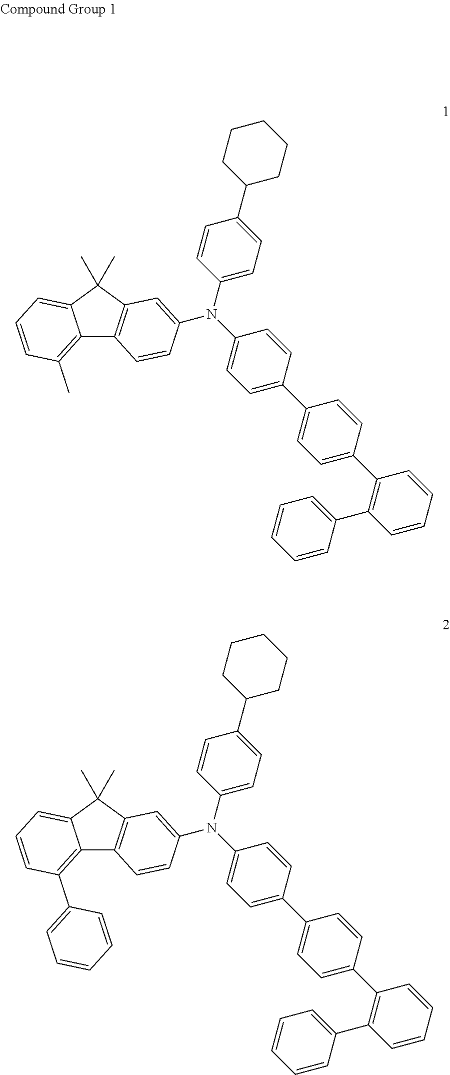 [SMILES: Cc1cccc2c1-c1ccc(N(c3ccc(-c4ccc(-c5ccccc5-c5ccccc5)cc4)cc3)c3ccc(C4CCCCC4)cc3)cc1C2(C)C ; CC1(C)c2cc(N(c3ccc(-c4ccc(-c5ccccc5-c5ccccc5)cc4)cc3)c3ccc(C4CCCCC4)cc3)ccc2-c2c(-c3ccccc3)cccc21]

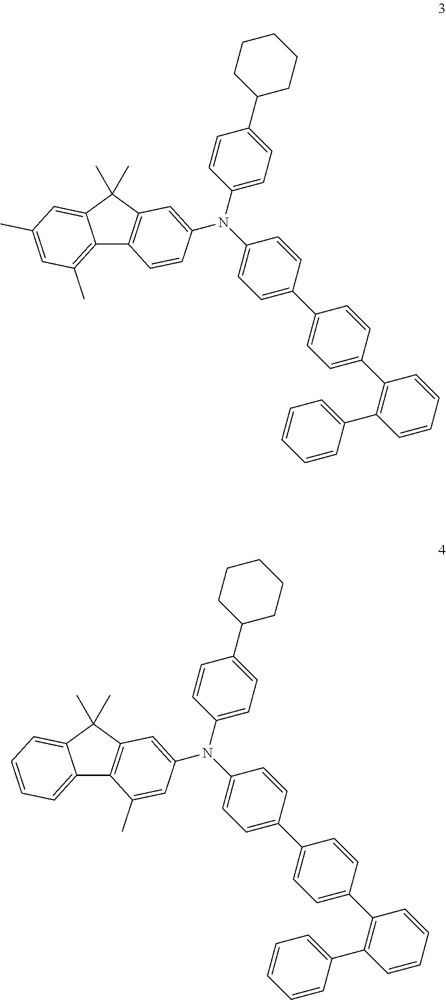 [SMILES: Cc1cc(C)c2c(c1)C(C)(C)c1cc(N(c3ccc(-c4ccc(-c5ccccc5-c5ccccc5)cc4)cc3)c3ccc(C4CCCCC4)cc3)ccc1-2 ; Cc1cc(N(c2ccc(-c3ccc(-c4ccccc4-c4ccccc4)cc3)cc2)c2ccc(C3CCCCC3)cc2)cc2c1-c1ccccc1C2(C)C]

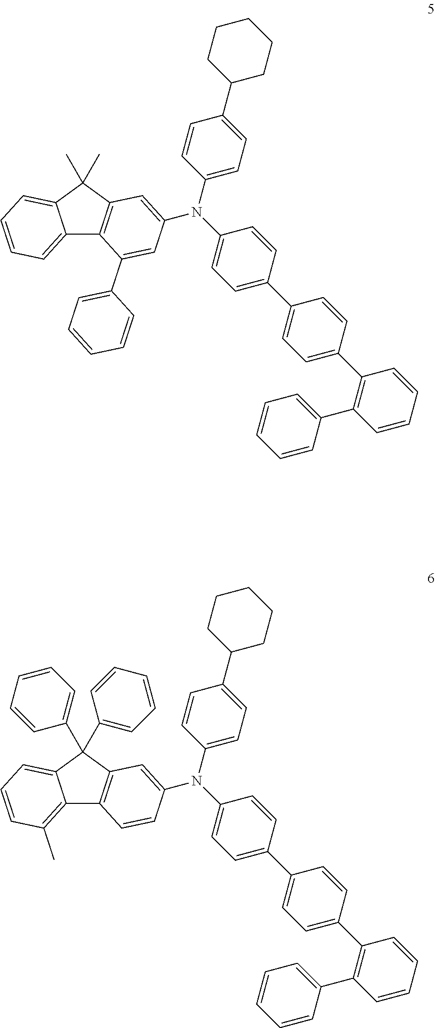 [SMILES: CC1(C)c2ccccc2-c2c(-c3ccccc3)cc(N(c3ccc(-c4ccc(-c5ccccc5-c5ccccc5)cc4)cc3)c3ccc(C4CCCCC4)cc3)cc21 ; Cc1cccc2c1-c1ccc(N(c3ccc(-c4ccc(-c5ccccc5-c5ccccc5)cc4)cc3)c3ccc(C4CCCCC4)cc3)cc1C2(c1ccccc1)c1ccccc1]

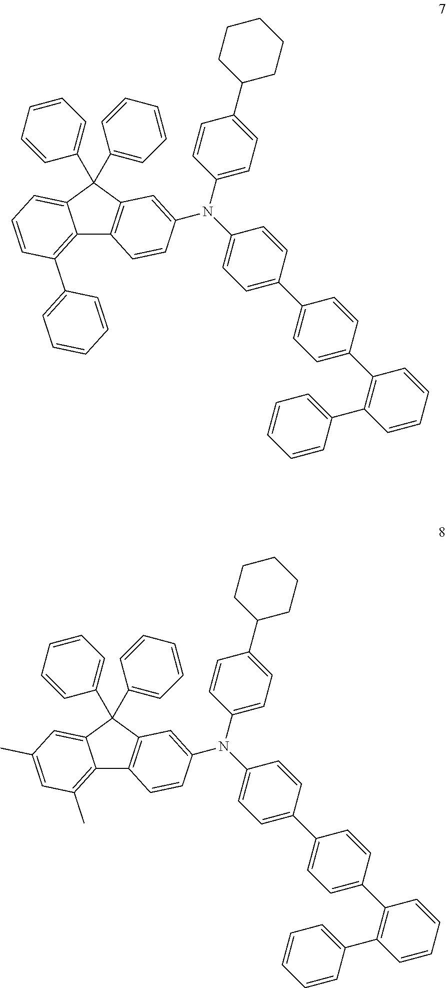 [SMILES: c1ccc(-c2ccccc2-c2ccc(-c3ccc(N(c4ccc(C5CCCCC5)cc4)c4ccc5c(c4)C(c4ccccc4)(c4ccccc4)c4cccc(-c6ccccc6)c4-5)cc3)cc2)cc1 ; Cc1cc(C)c2c(c1)C(c1ccccc1)(c1ccccc1)c1cc(N(c3ccc(-c4ccc(-c5ccccc5-c5ccccc5)cc4)cc3)c3ccc(C4CCCCC4)cc3)ccc1-2]

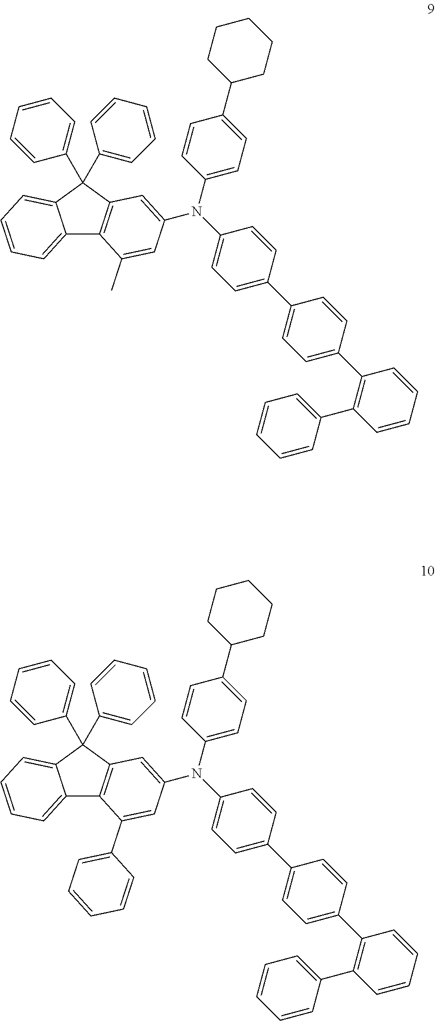 [SMILES: Cc1cc(N(c2ccc(-c3ccc(-c4ccccc4-c4ccccc4)cc3)cc2)c2ccc(C3CCCCC3)cc2)cc2c1-c1ccccc1C2(c1ccccc1)c1ccccc1 ; c1ccc(-c2ccccc2-c2ccc(-c3ccc(N(c4ccc(C5CCCCC5)cc4)c4cc(-c5ccccc5)c5c(c4)C(c4ccccc4)(c4ccccc4)c4ccccc4-5)cc3)cc2)cc1]

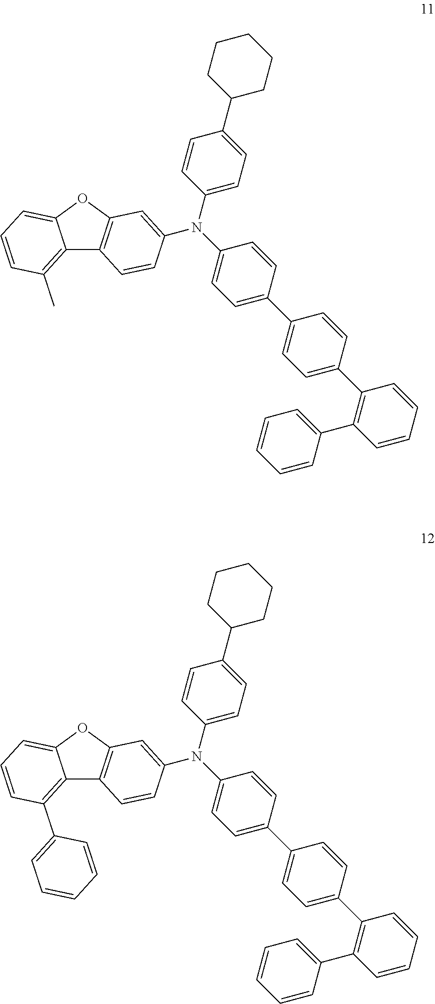 [SMILES: Cc1cccc2oc3cc(N(c4ccc(-c5ccc(-c6ccccc6-c6ccccc6)cc5)cc4)c4ccc(C5CCCCC5)cc4)ccc3c12 ; c1ccc(-c2ccccc2-c2ccc(-c3ccc(N(c4ccc(C5CCCCC5)cc4)c4ccc5c(c4)oc4cccc(-c6ccccc6)c45)cc3)cc2)cc1]

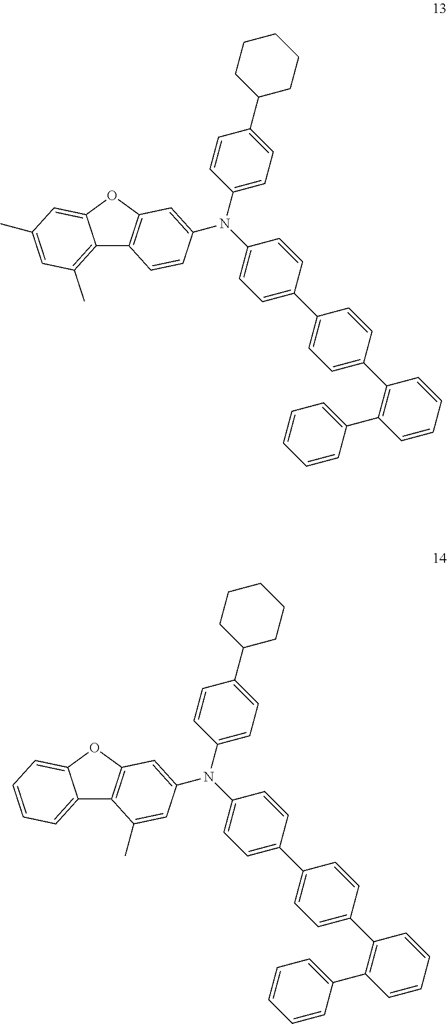 [SMILES: Cc1cc(C)c2c(c1)oc1cc(N(c3ccc(-c4ccc(-c5ccccc5-c5ccccc5)cc4)cc3)c3ccc(C4CCCCC4)cc3)ccc12 ; Cc1cc(N(c2ccc(-c3ccc(-c4ccccc4-c4ccccc4)cc3)cc2)c2ccc(C3CCCCC3)cc2)cc2oc3ccccc3c12]

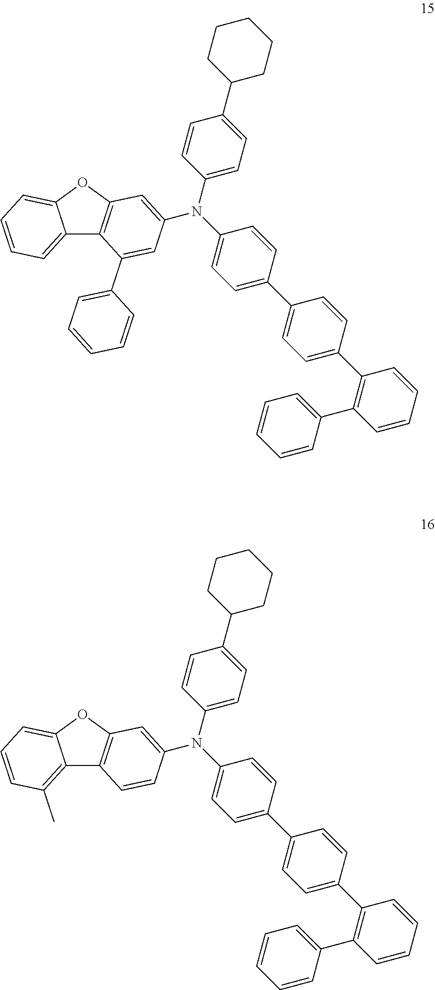 [SMILES: c1ccc(-c2ccccc2-c2ccc(-c3ccc(N(c4ccc(C5CCCCC5)cc4)c4cc(-c5ccccc5)c5c(c4)oc4ccccc45)cc3)cc2)cc1 ; Cc1cccc2oc3cc(N(c4ccc(-c5ccc(-c6ccccc6-c6ccccc6)cc5)cc4)c4ccc(C5CCCCC5)cc4)ccc3c12]

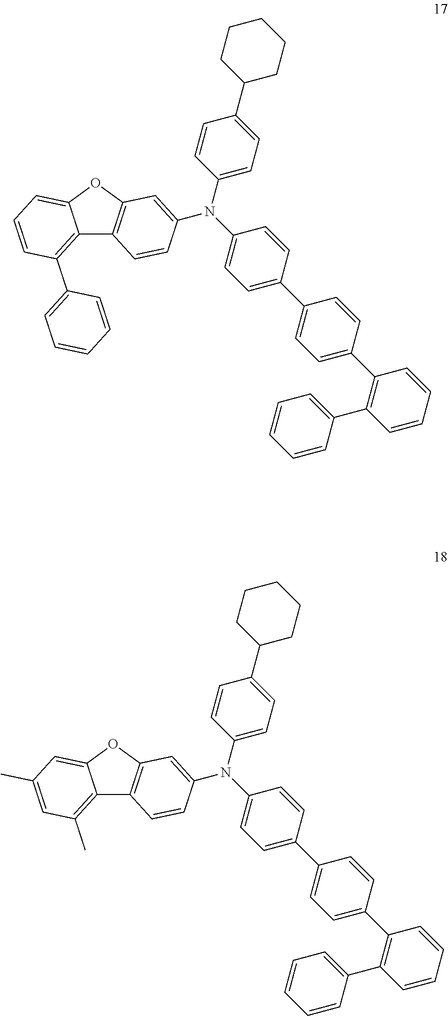 [SMILES: c1ccc(-c2ccccc2-c2ccc(-c3ccc(N(c4ccc(C5CCCCC5)cc4)c4ccc5c(c4)oc4cccc(-c6ccccc6)c45)cc3)cc2)cc1 ; Cc1cc(C)c2c(c1)oc1cc(N(c3ccc(-c4ccc(-c5ccccc5-c5ccccc5)cc4)cc3)c3ccc(C4CCCCC4)cc3)ccc12]

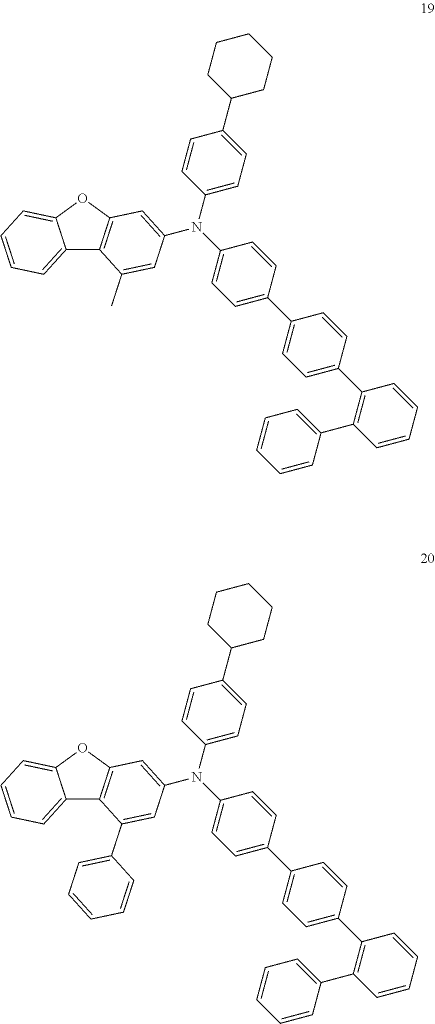 [SMILES: Cc1cc(N(c2ccc(-c3ccc(-c4ccccc4-c4ccccc4)cc3)cc2)c2ccc(C3CCCCC3)cc2)cc2oc3ccccc3c12 ; c1ccc(-c2ccccc2-c2ccc(-c3ccc(N(c4ccc(C5CCCCC5)cc4)c4cc(-c5ccccc5)c5c(c4)oc4ccccc45)cc3)cc2)cc1]

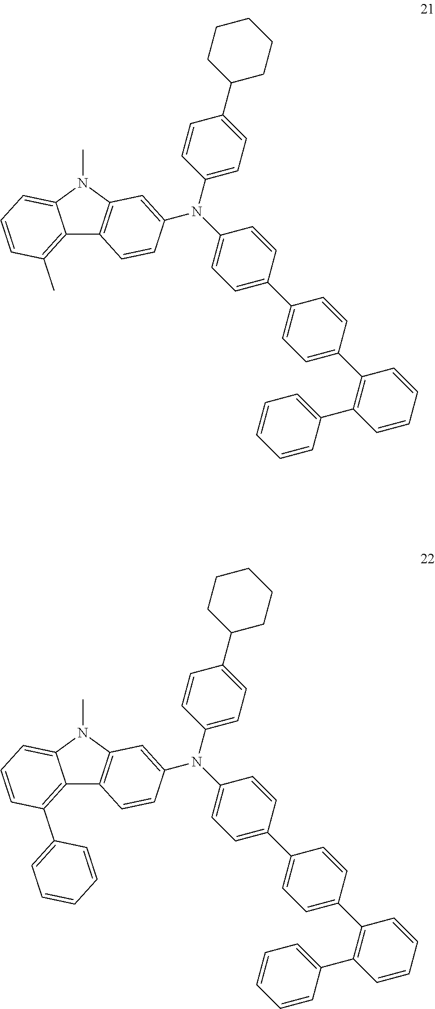 [SMILES: Cc1cccc2c1c1ccc(N(c3ccc(-c4ccc(-c5ccccc5-c5ccccc5)cc4)cc3)c3ccc(C4CCCCC4)cc3)cc1n2C ; Cn1c2cc(N(c3ccc(-c4ccc(-c5ccccc5-c5ccccc5)cc4)cc3)c3ccc(C4CCCCC4)cc3)ccc2c2c(-c3ccccc3)cccc21]

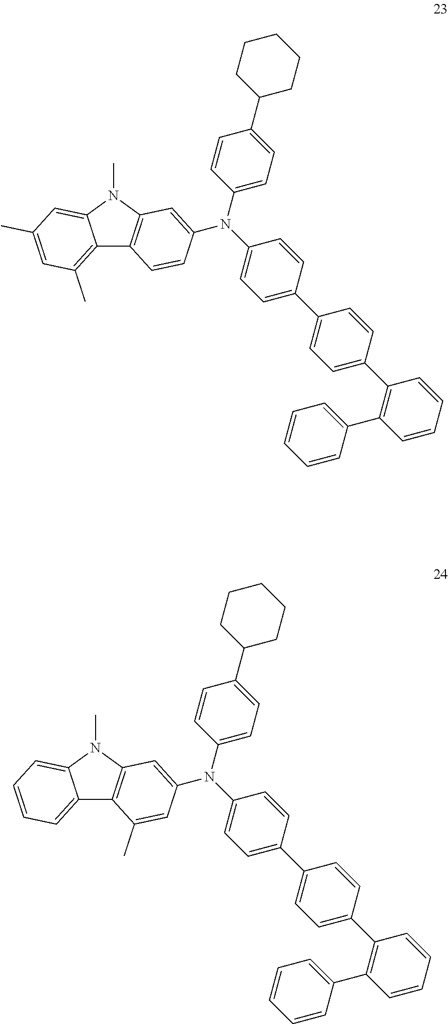 [SMILES: Cc1cc(C)c2c3ccc(N(c4ccc(-c5ccc(-c6ccccc6-c6ccccc6)cc5)cc4)c4ccc(C5CCCCC5)cc4)cc3n(C)c2c1 ; Cc1cc(N(c2ccc(-c3ccc(-c4ccccc4-c4ccccc4)cc3)cc2)c2ccc(C3CCCCC3)cc2)cc2c1c1ccccc1n2C]

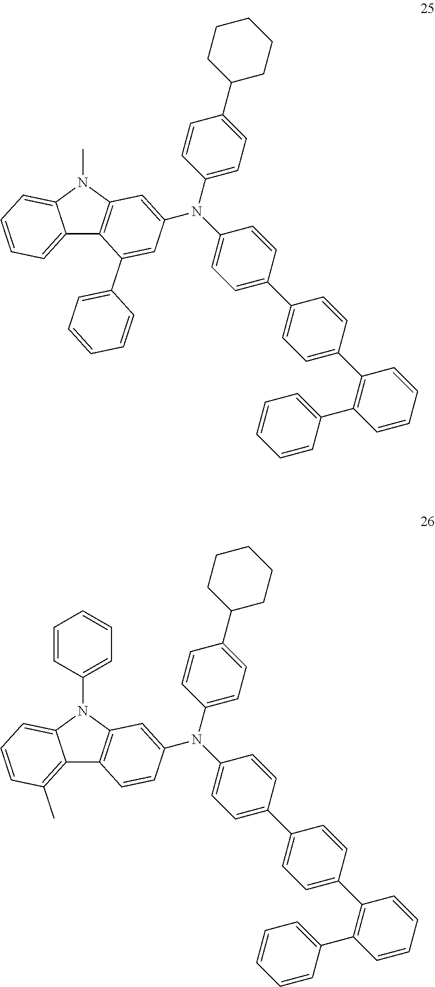 [SMILES: Cn1c2ccccc2c2c(-c3ccccc3)cc(N(c3ccc(-c4ccc(-c5ccccc5-c5ccccc5)cc4)cc3)c3ccc(C4CCCCC4)cc3)cc21 ; Cc1cccc2c1c1ccc(N(c3ccc(-c4ccc(-c5ccccc5-c5ccccc5)cc4)cc3)c3ccc(C4CCCCC4)cc3)cc1n2-c1ccccc1]

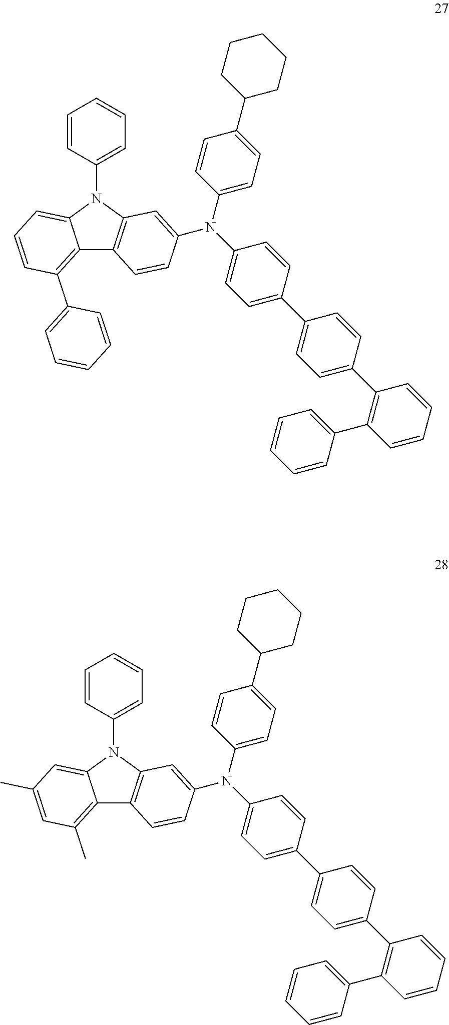 [SMILES: c1ccc(-c2ccccc2-c2ccc(-c3ccc(N(c4ccc(C5CCCCC5)cc4)c4ccc5c6c(-c7ccccc7)cccc6n(-c6ccccc6)c5c4)cc3)cc2)cc1 ; Cc1cc(C)c2c3ccc(N(c4ccc(-c5ccc(-c6ccccc6-c6ccccc6)cc5)cc4)c4ccc(C5CCCCC5)cc4)cc3n(-c3ccccc3)c2c1]

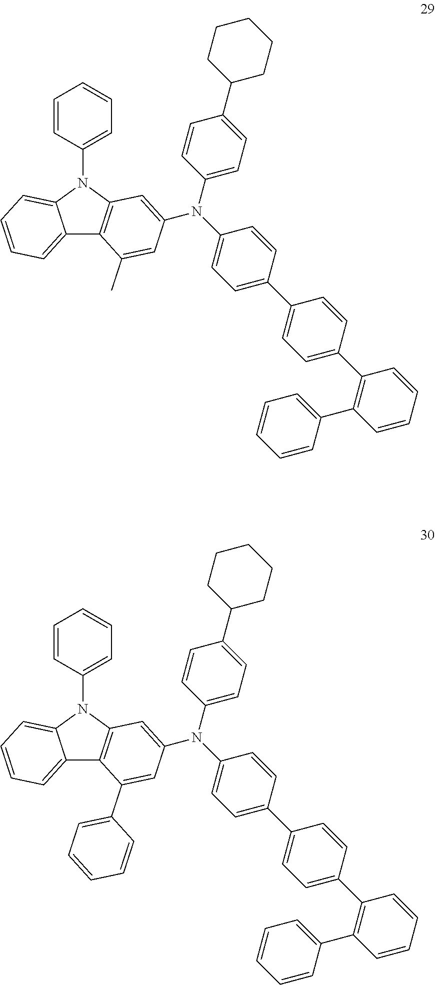 [SMILES: Cc1cc(N(c2ccc(-c3ccc(-c4ccccc4-c4ccccc4)cc3)cc2)c2ccc(C3CCCCC3)cc2)cc2c1c1ccccc1n2-c1ccccc1 ; c1ccc(-c2ccccc2-c2ccc(-c3ccc(N(c4ccc(C5CCCCC5)cc4)c4cc(-c5ccccc5)c5c6ccccc6n(-c6ccccc6)c5c4)cc3)cc2)cc1]

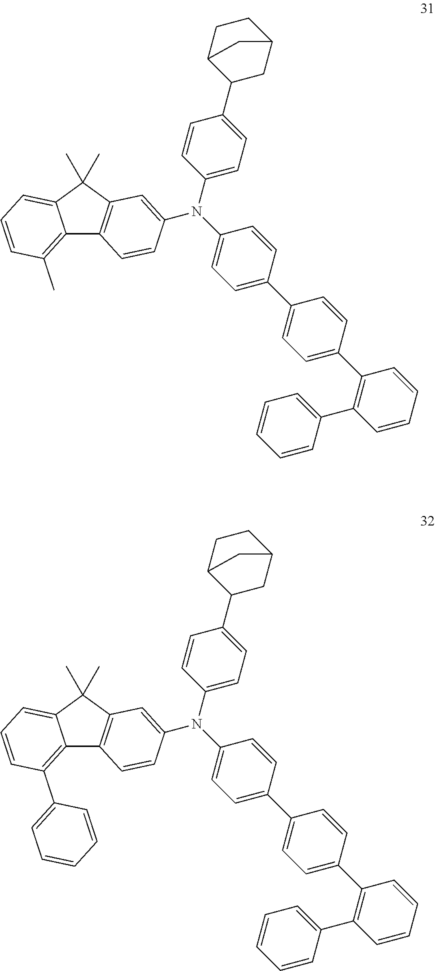 [SMILES: Cc1cccc2c1-c1ccc(N(c3ccc(-c4ccc(-c5ccccc5-c5ccccc5)cc4)cc3)c3ccc(C4CC5CCC4C5)cc3)cc1C2(C)C ; CC1(C)c2cc(N(c3ccc(-c4ccc(-c5ccccc5-c5ccccc5)cc4)cc3)c3ccc(C4CC5CCC4C5)cc3)ccc2-c2c(-c3ccccc3)cccc21]

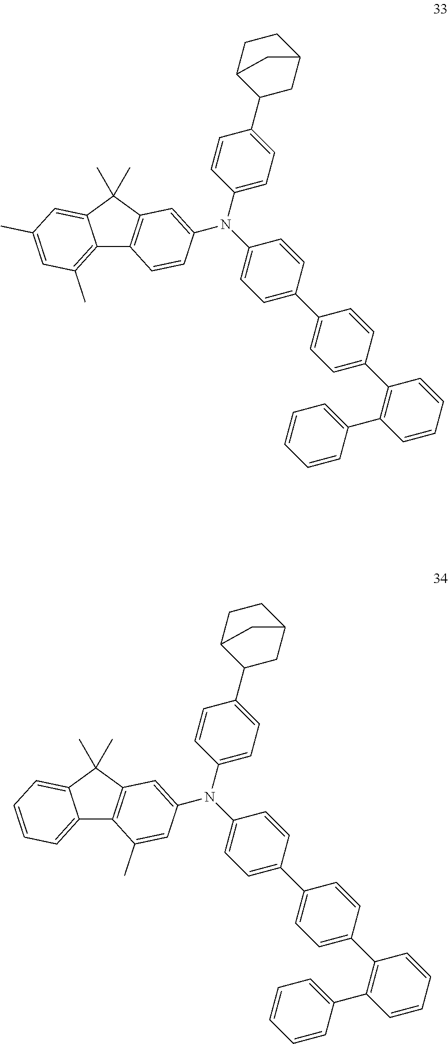 [SMILES: Cc1cc(C)c2c(c1)C(C)(C)c1cc(N(c3ccc(-c4ccc(-c5ccccc5-c5ccccc5)cc4)cc3)c3ccc(C4CC5CCC4C5)cc3)ccc1-2 ; Cc1cc(N(c2ccc(-c3ccc(-c4ccccc4-c4ccccc4)cc3)cc2)c2ccc(C3CC4CCC3C4)cc2)cc2c1-c1ccccc1C2(C)C]

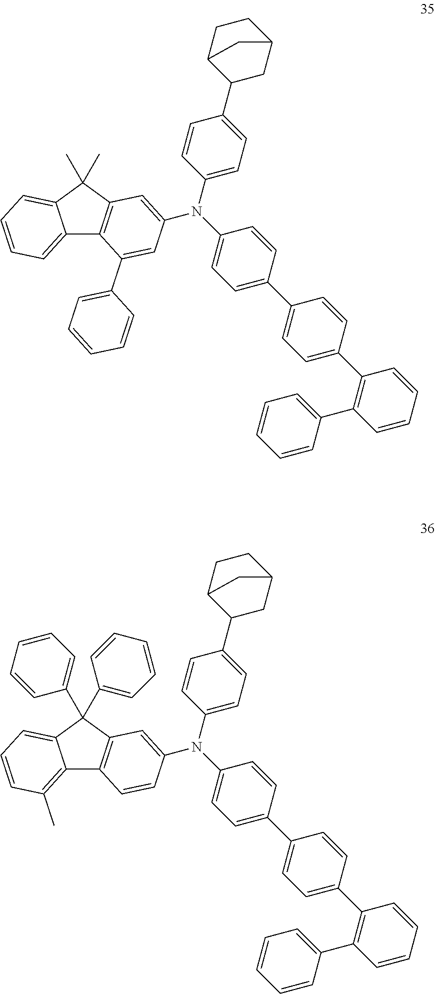 [SMILES: CC1(C)c2ccccc2-c2c(-c3ccccc3)cc(N(c3ccc(-c4ccc(-c5ccccc5-c5ccccc5)cc4)cc3)c3ccc(C4CC5CCC4C5)cc3)cc21 ; Cc1cccc2c1-c1ccc(N(c3ccc(-c4ccc(-c5ccccc5-c5ccccc5)cc4)cc3)c3ccc(C4CC5CCC4C5)cc3)cc1C2(c1ccccc1)c1ccccc1]

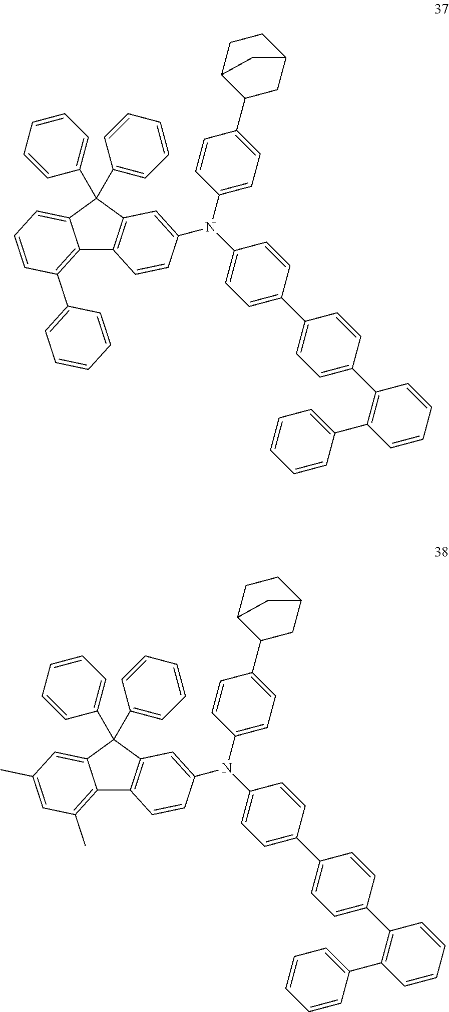 [SMILES: c1ccc(-c2ccccc2-c2ccc(-c3ccc(N(c4ccc(C5CC6CCC5C6)cc4)c4ccc5c(c4)C(c4ccccc4)(c4ccccc4)c4cccc(-c6ccccc6)c4-5)cc3)cc2)cc1 ; Cc1cc(C)c2c(c1)C(c1ccccc1)(c1ccccc1)c1cc(N(c3ccc(-c4ccc(-c5ccccc5-c5ccccc5)cc4)cc3)c3ccc(C4CC5CCC4C5)cc3)ccc1-2]

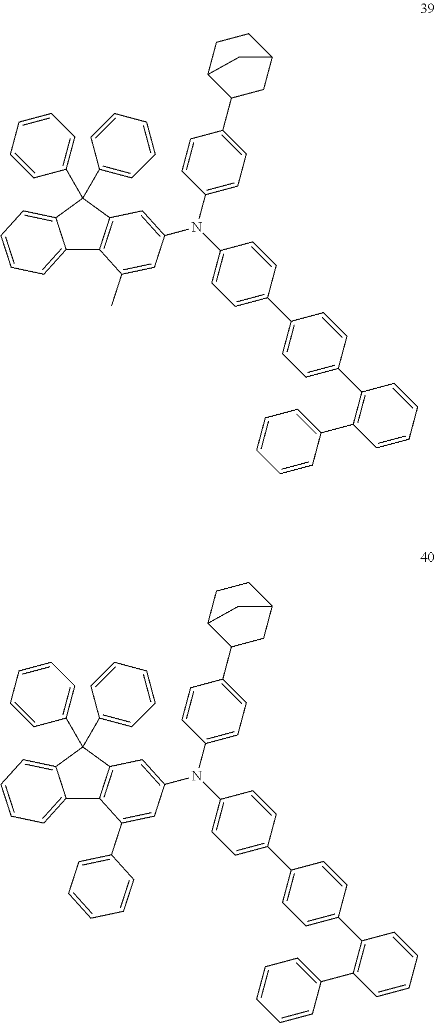 [SMILES: Cc1cc(N(c2ccc(-c3ccc(-c4ccccc4-c4ccccc4)cc3)cc2)c2ccc(C3CC4CCC3C4)cc2)cc2c1-c1ccccc1C2(c1ccccc1)c1ccccc1 ; c1ccc(-c2ccccc2-c2ccc(-c3ccc(N(c4ccc(C5CC6CCC5C6)cc4)c4cc(-c5ccccc5)c5c(c4)C(c4ccccc4)(c4ccccc4)c4ccccc4-5)cc3)cc2)cc1]

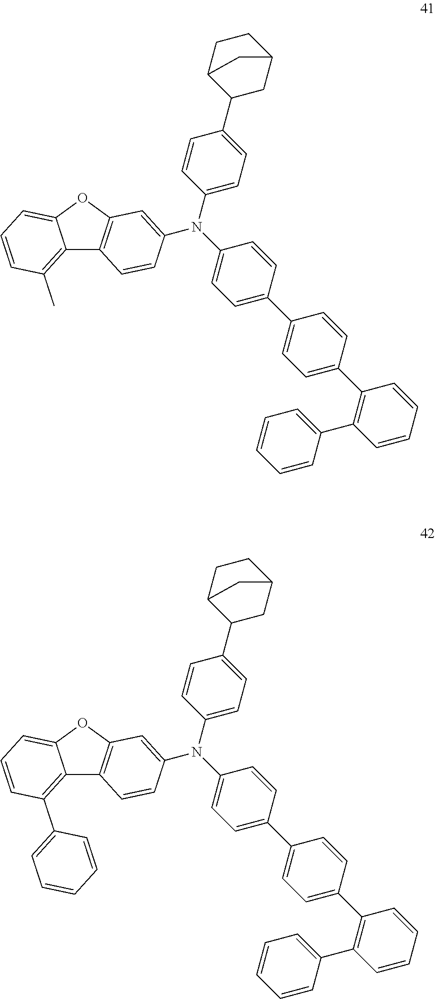 [SMILES: Cc1cccc2oc3cc(N(c4ccc(-c5ccc(-c6ccccc6-c6ccccc6)cc5)cc4)c4ccc(C5CC6CCC5C6)cc4)ccc3c12 ; c1ccc(-c2ccccc2-c2ccc(-c3ccc(N(c4ccc(C5CC6CCC5C6)cc4)c4ccc5c(c4)oc4cccc(-c6ccccc6)c45)cc3)cc2)cc1]

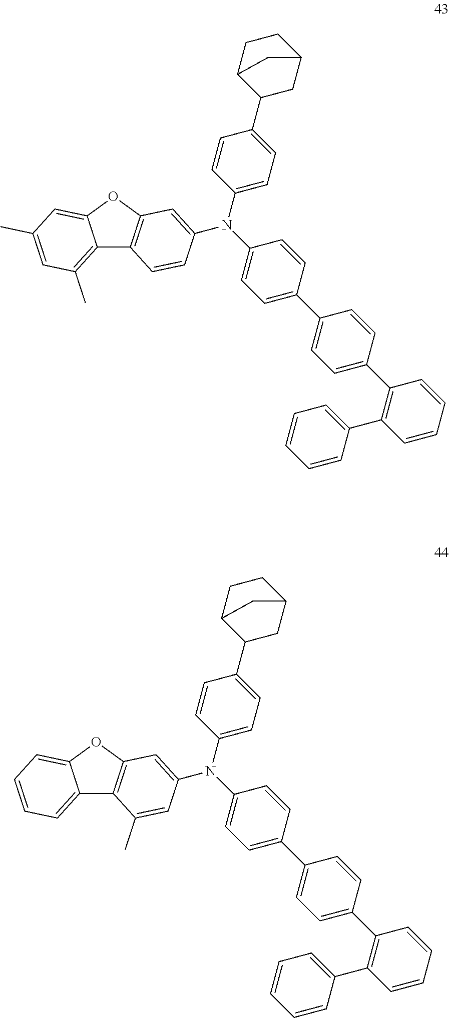 [SMILES: Cc1cc(C)c2c(c1)oc1cc(N(c3ccc(-c4ccc(-c5ccccc5-c5ccccc5)cc4)cc3)c3ccc(C4CC5CCC4C5)cc3)ccc12 ; Cc1cc(N(c2ccc(-c3ccc(-c4ccccc4-c4ccccc4)cc3)cc2)c2ccc(C3CC4CCC3C4)cc2)cc2oc3ccccc3c12]

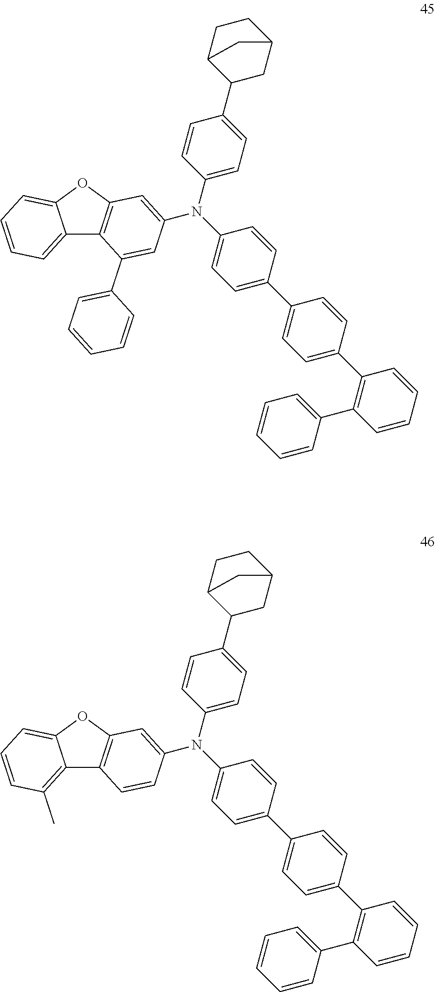 [SMILES: c1ccc(-c2ccccc2-c2ccc(-c3ccc(N(c4ccc(C5CC6CCC5C6)cc4)c4cc(-c5ccccc5)c5c(c4)oc4ccccc45)cc3)cc2)cc1 ; Cc1cccc2oc3cc(N(c4ccc(-c5ccc(-c6ccccc6-c6ccccc6)cc5)cc4)c4ccc(C5CC6CCC5C6)cc4)ccc3c12]

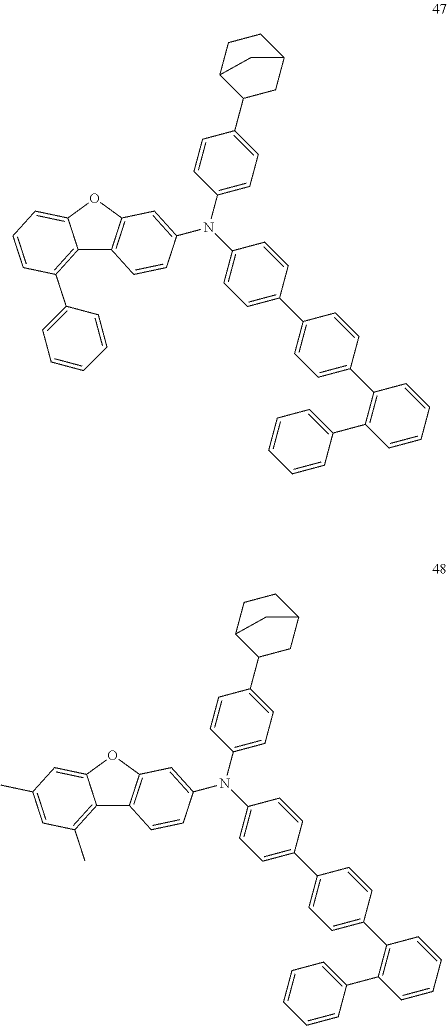 [SMILES: c1ccc(-c2ccccc2-c2ccc(-c3ccc(N(c4ccc(C5CC6CCC5C6)cc4)c4ccc5c(c4)oc4cccc(-c6ccccc6)c45)cc3)cc2)cc1 ; Cc1cc(C)c2c(c1)oc1cc(N(c3ccc(-c4ccc(-c5ccccc5-c5ccccc5)cc4)cc3)c3ccc(C4CC5CCC4C5)cc3)ccc12]

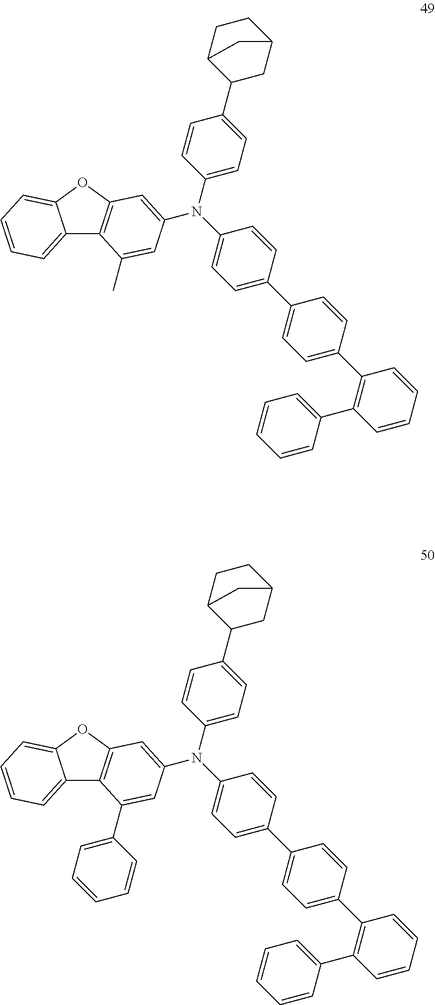 [SMILES: Cc1cc(N(c2ccc(-c3ccc(-c4ccccc4-c4ccccc4)cc3)cc2)c2ccc(C3CC4CCC3C4)cc2)cc2oc3ccccc3c12 ; c1ccc(-c2ccccc2-c2ccc(-c3ccc(N(c4ccc(C5CC6CCC5C6)cc4)c4cc(-c5ccccc5)c5c(c4)oc4ccccc45)cc3)cc2)cc1]

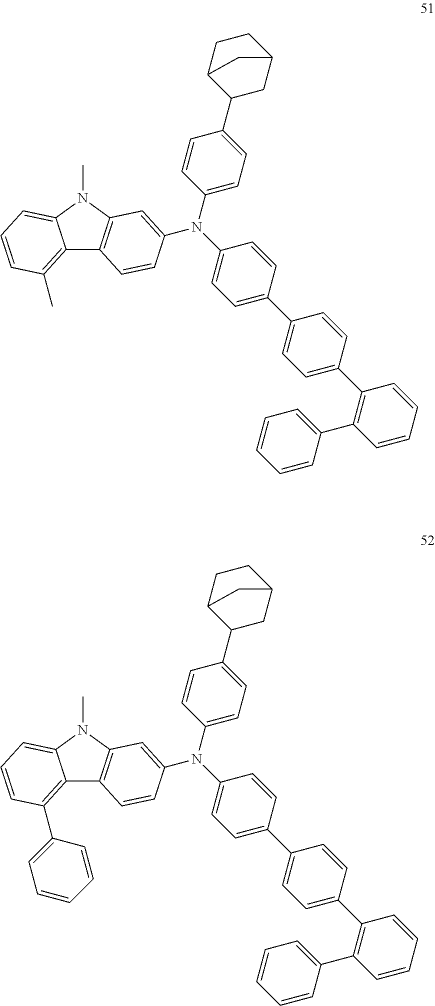 [SMILES: Cc1cccc2c1c1ccc(N(c3ccc(-c4ccc(-c5ccccc5-c5ccccc5)cc4)cc3)c3ccc(C4CC5CCC4C5)cc3)cc1n2C ; Cn1c2cc(N(c3ccc(-c4ccc(-c5ccccc5-c5ccccc5)cc4)cc3)c3ccc(C4CC5CCC4C5)cc3)ccc2c2c(-c3ccccc3)cccc21]

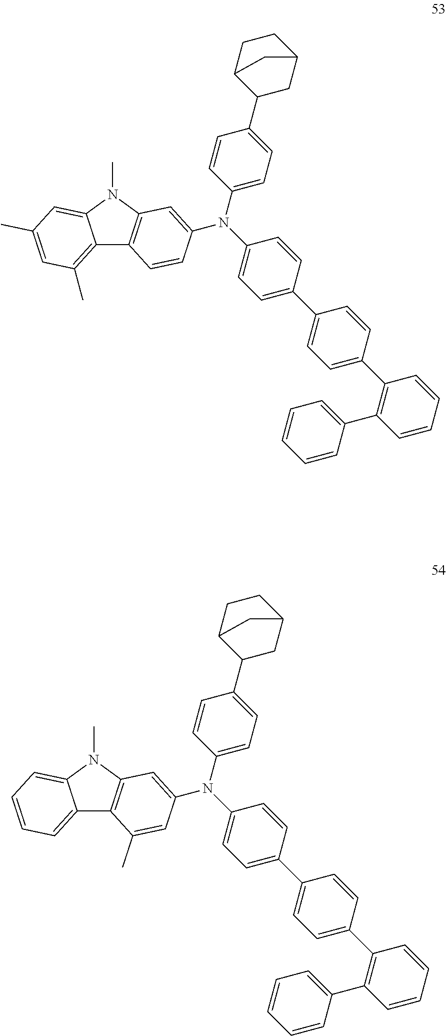 [SMILES: Cc1cc(C)c2c3ccc(N(c4ccc(-c5ccc(-c6ccccc6-c6ccccc6)cc5)cc4)c4ccc(C5CC6CCC5C6)cc4)cc3n(C)c2c1 ; Cc1cc(N(c2ccc(-c3ccc(-c4ccccc4-c4ccccc4)cc3)cc2)c2ccc(C3CC4CCC3C4)cc2)cc2c1c1ccccc1n2C]

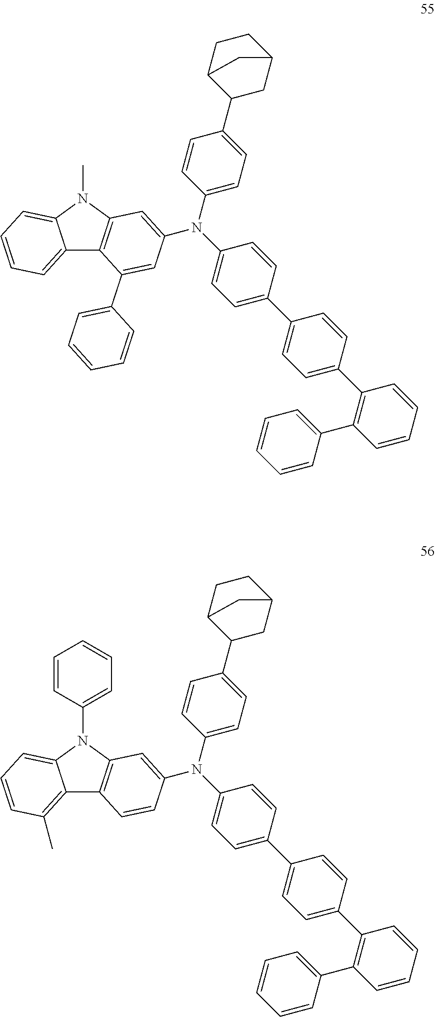 [SMILES: Cn1c2ccccc2c2c(-c3ccccc3)cc(N(c3ccc(-c4ccc(-c5ccccc5-c5ccccc5)cc4)cc3)c3ccc(C4CC5CCC4C5)cc3)cc21 ; Cc1cccc2c1c1ccc(N(c3ccc(-c4ccc(-c5ccccc5-c5ccccc5)cc4)cc3)c3ccc(C4CC5CCC4C5)cc3)cc1n2-c1ccccc1]

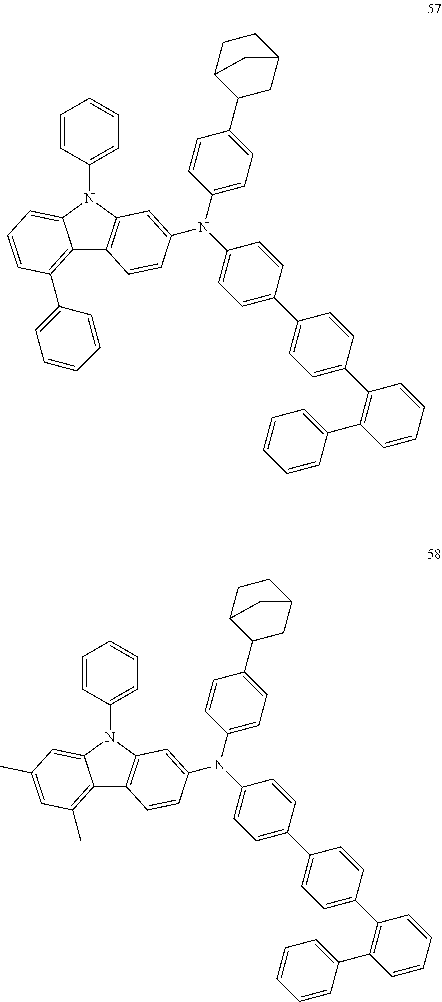 [SMILES: c1ccc(-c2ccccc2-c2ccc(-c3ccc(N(c4ccc(C5CC6CCC5C6)cc4)c4ccc5c6c(-c7ccccc7)cccc6n(-c6ccccc6)c5c4)cc3)cc2)cc1 ; Cc1cc(C)c2c3ccc(N(c4ccc(-c5ccc(-c6ccccc6-c6ccccc6)cc5)cc4)c4ccc(C5CC6CCC5C6)cc4)cc3n(-c3ccccc3)c2c1]

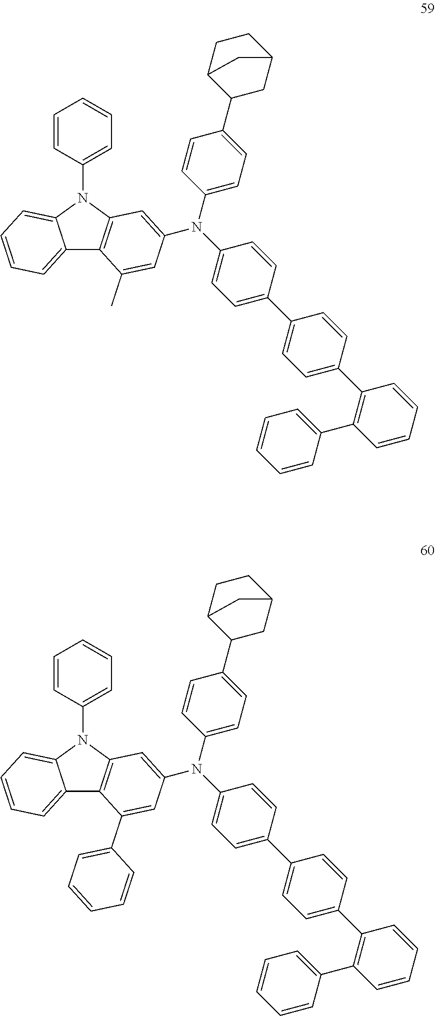 [SMILES: Cc1cc(N(c2ccc(-c3ccc(-c4ccccc4-c4ccccc4)cc3)cc2)c2ccc(C3CC4CCC3C4)cc2)cc2c1c1ccccc1n2-c1ccccc1 ; c1ccc(-c2ccccc2-c2ccc(-c3ccc(N(c4ccc(C5CC6CCC5C6)cc4)c4cc(-c5ccccc5)c5c6ccccc6n(-c6ccccc6)c5c4)cc3)cc2)cc1]

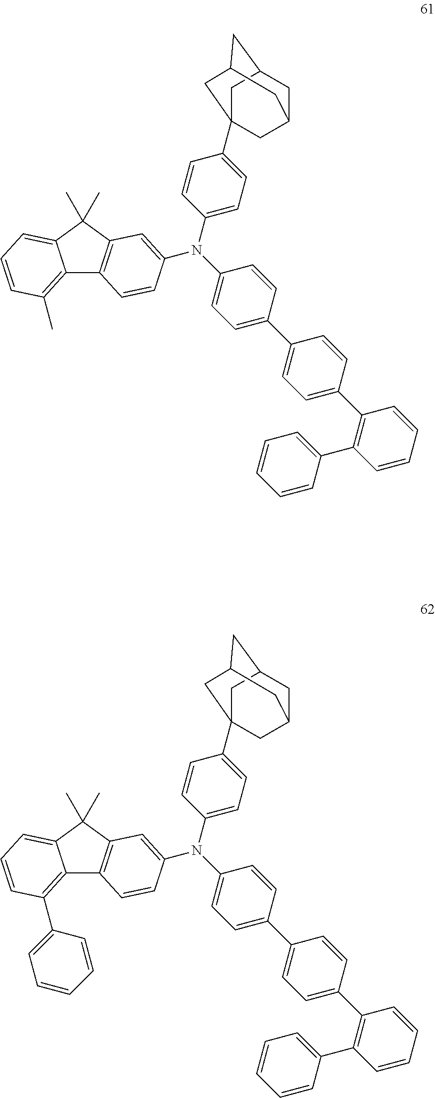 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(C)C ; CC1(C)c2cc(N(c3ccc(-c4ccc(-c5ccccc5-c5ccccc5)cc4)cc3)c3ccc(C45CC6CC(CC(C6)C4)C5)cc3)ccc2-c2c(-c3ccccc3)cccc21]

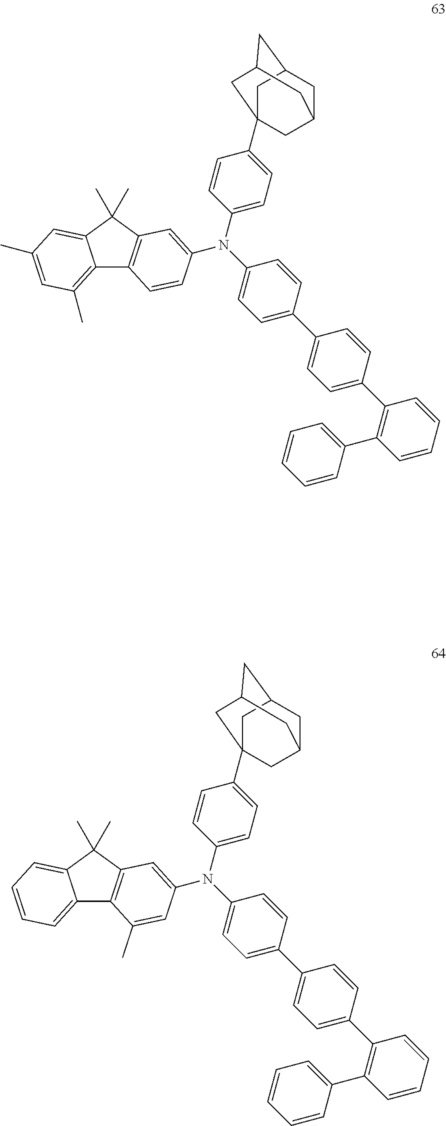 [SMILES: Cc1cc(C)c2c(c1)C(C)(C)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(C)C]

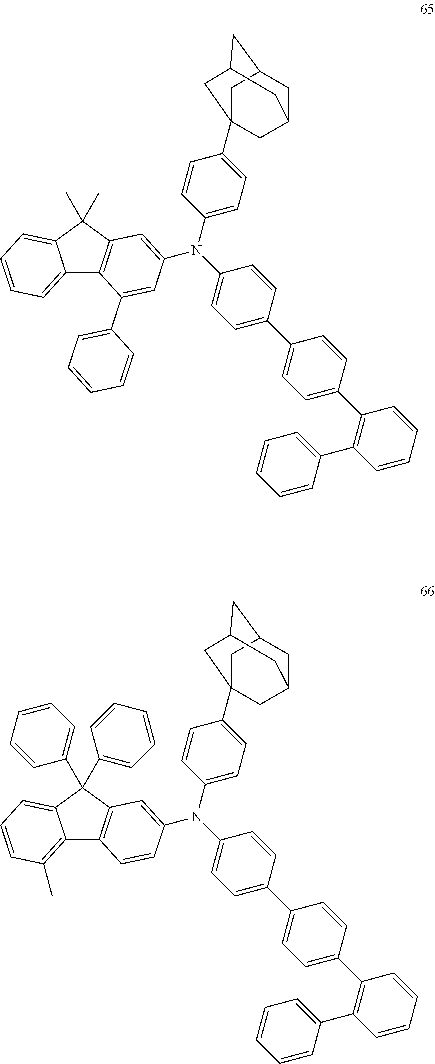 [SMILES: CC1(C)c2ccccc2-c2c(-c3ccccc3)cc(N(c3ccc(-c4ccc(-c5ccccc5-c5ccccc5)cc4)cc3)c3ccc(C45CC6CC(CC(C6)C4)C5)cc3)cc21 ; Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(c1ccccc1)c1ccccc1]

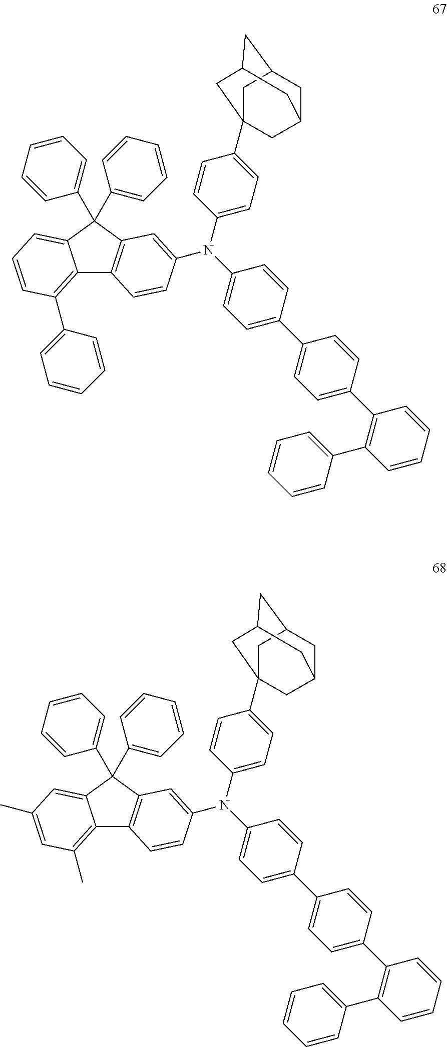 [SMILES: c1ccc(-c2ccccc2-c2ccc(-c3ccc(N(c4ccc(C56CC7CC(CC(C7)C5)C6)cc4)c4ccc5c(c4)C(c4ccccc4)(c4ccccc4)c4cccc(-c6ccccc6)c4-5)cc3)cc2)cc1 ; Cc1cc(C)c2c(c1)C(c1ccccc1)(c1ccccc1)c1cc(N(c3ccc(-c4ccc(-c5ccccc5-c5ccccc5)cc4)cc3)c3ccc(C45CC6CC(CC(C6)C4)C5)cc3)ccc1-2]

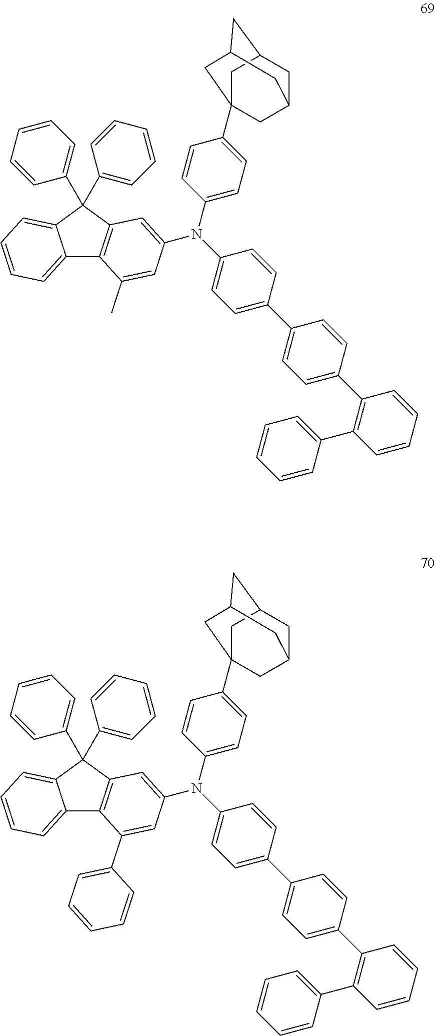 [SMILES: Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(c1ccccc1)c1ccccc1 ; c1ccc(-c2ccccc2-c2ccc(-c3ccc(N(c4ccc(C56CC7CC(CC(C7)C5)C6)cc4)c4cc(-c5ccccc5)c5c(c4)C(c4ccccc4)(c4ccccc4)c4ccccc4-5)cc3)cc2)cc1]

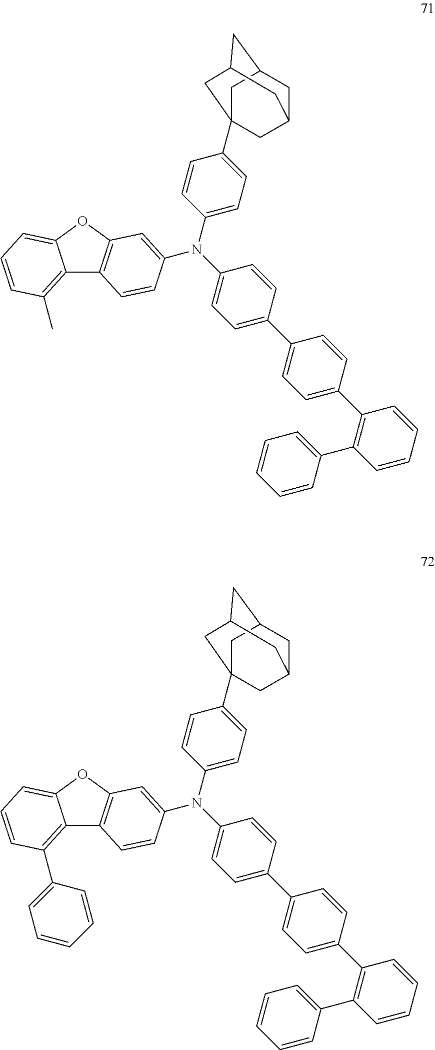 [SMILES: Cc1cccc2oc3cc(N(c4ccc(-c5ccc(-c6ccccc6-c6ccccc6)cc5)cc4)c4ccc(C56CC7CC(CC(C7)C5)C6)cc4)ccc3c12 ; c1ccc(-c2ccccc2-c2ccc(-c3ccc(N(c4ccc(C56CC7CC(CC(C7)C5)C6)cc4)c4ccc5c(c4)oc4cccc(-c6ccccc6)c45)cc3)cc2)cc1]

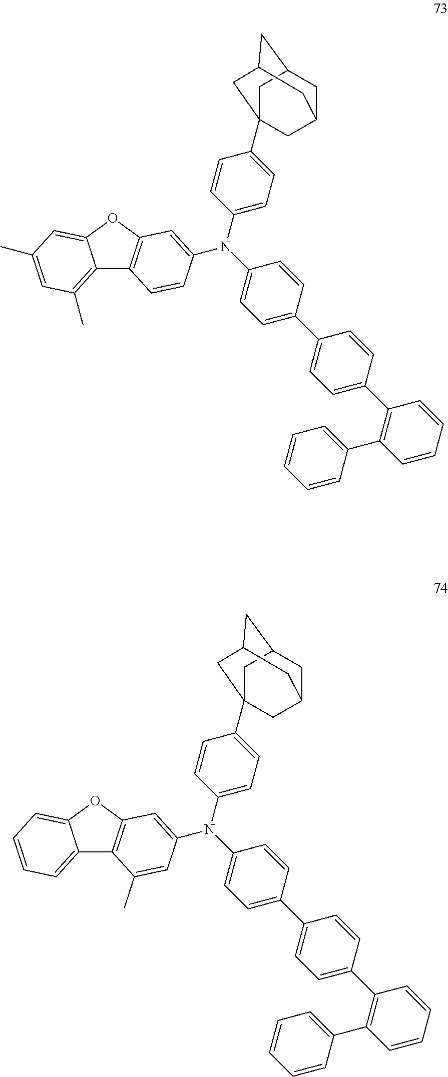 [SMILES: Cc1cc(C)c2c(c1)oc1cc(N(c3ccc(-c4ccc(-c5ccccc5-c5ccccc5)cc4)cc3)c3ccc(C45CC6CC(CC(C6)C4)C5)cc3)ccc12 ; Cc1cc(N(c2ccc(-c3ccc(-c4ccccc4-c4ccccc4)cc3)cc2)c2ccc(C34CC5CC(CC(C5)C3)C4)cc2)cc2oc3ccccc3c12]

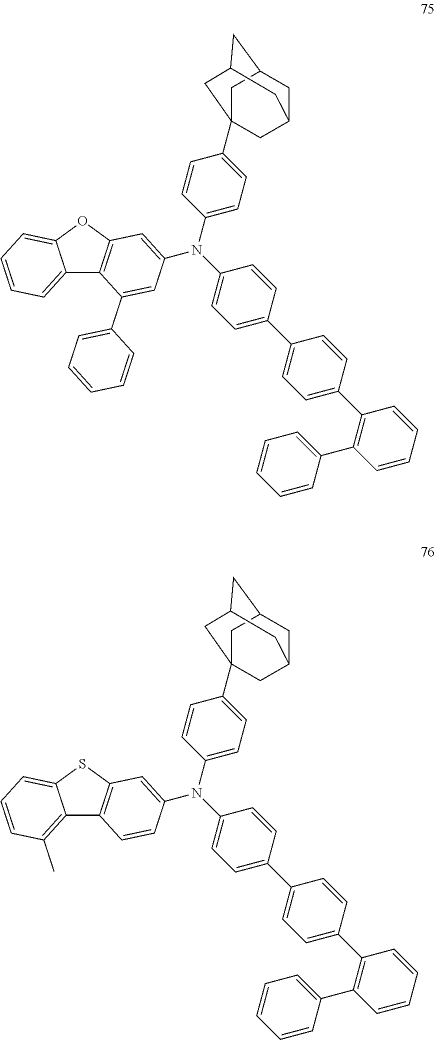 [SMILES: c1ccc(-c2ccccc2-c2ccc(-c3ccc(N(c4ccc(C56CC7CC(CC(C7)C5)C6)cc4)c4cc(-c5ccccc5)c5c(c4)oc4ccccc45)cc3)cc2)cc1 ; Cc1cccc2sc3cc(N(c4ccc(-c5ccc(-c6ccccc6-c6ccccc6)cc5)cc4)c4ccc(C56CC7CC(CC(C7)C5)C6)cc4)ccc3c12]

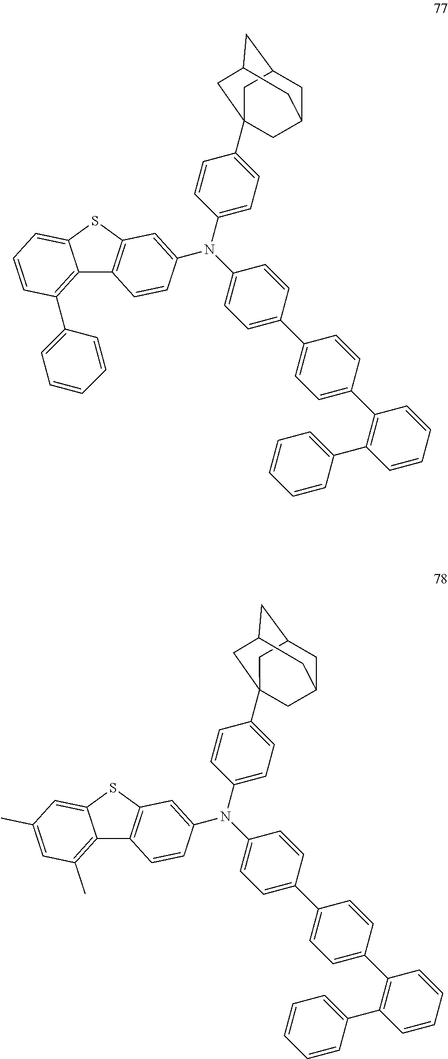 [SMILES: c1ccc(-c2ccccc2-c2ccc(-c3ccc(N(c4ccc(C56CC7CC(CC(C7)C5)C6)cc4)c4ccc5c(c4)sc4cccc(-c6ccccc6)c45)cc3)cc2)cc1 ; Cc1cc(C)c2c(c1)sc1cc(N(c3ccc(-c4ccc(-c5ccccc5-c5ccccc5)cc4)cc3)c3ccc(C45CC6CC(CC(C6)C4)C5)cc3)ccc12]

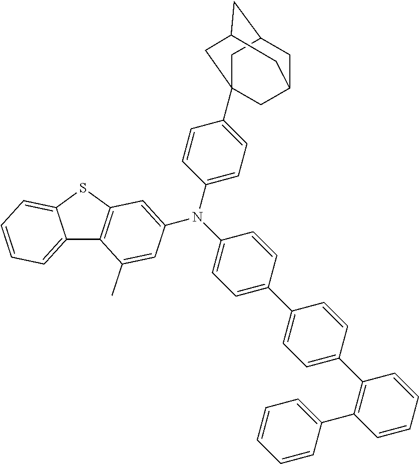 [SMILES: Cc1cc(N(c2ccc(-c3ccc(-c4ccccc4-c4ccccc4)cc3)cc2)c2ccc(C34CC5CC(CC(C5)C3)C4)cc2)cc2sc3ccccc3c12]

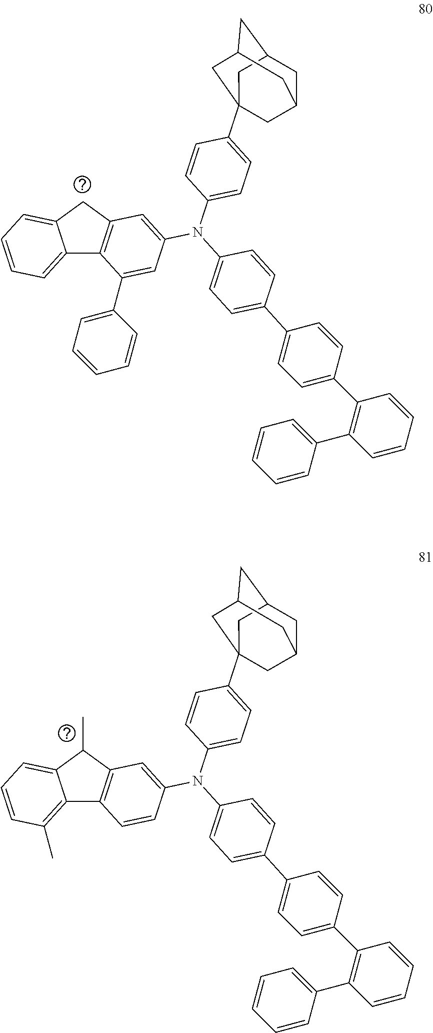 [SMILES: c1ccc(-c2ccccc2-c2ccc(-c3ccc(N(c4ccc(C56CC7CC(CC(C7)C5)C6)cc4)c4cc5c(c(-c6ccccc6)c4)-c4ccccc4C5)cc3)cc2)cc1 ; Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2C]

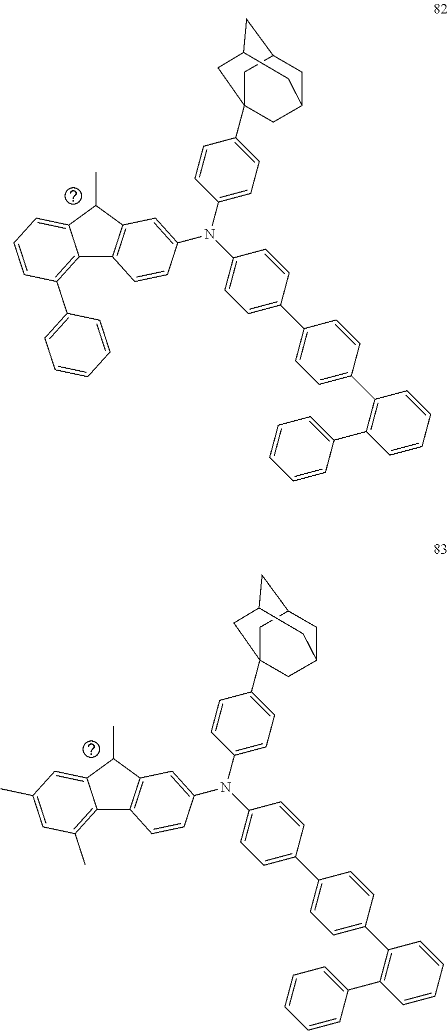 [SMILES: CC1c2cc(N(c3ccc(-c4ccc(-c5ccccc5-c5ccccc5)cc4)cc3)c3ccc(C45CC6CC(CC(C6)C4)C5)cc3)ccc2-c2c(-c3ccccc3)cccc21 ; Cc1cc(C)c2c(c1)C(C)c1cc(N(c3ccc(-c4ccc(-c5ccccc5-c5ccccc5)cc4)cc3)c3ccc(C45CC6CC(CC(C6)C4)C5)cc3)ccc1-2]

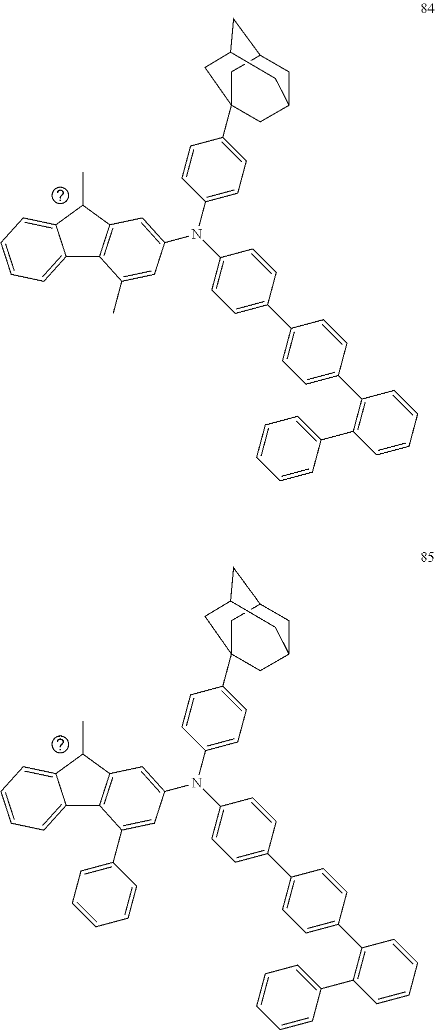 [SMILES: Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2C ; CC1c2ccccc2-c2c(-c3ccccc3)cc(N(c3ccc(-c4ccc(-c5ccccc5-c5ccccc5)cc4)cc3)c3ccc(C45CC6CC(CC(C6)C4)C5)cc3)cc21]

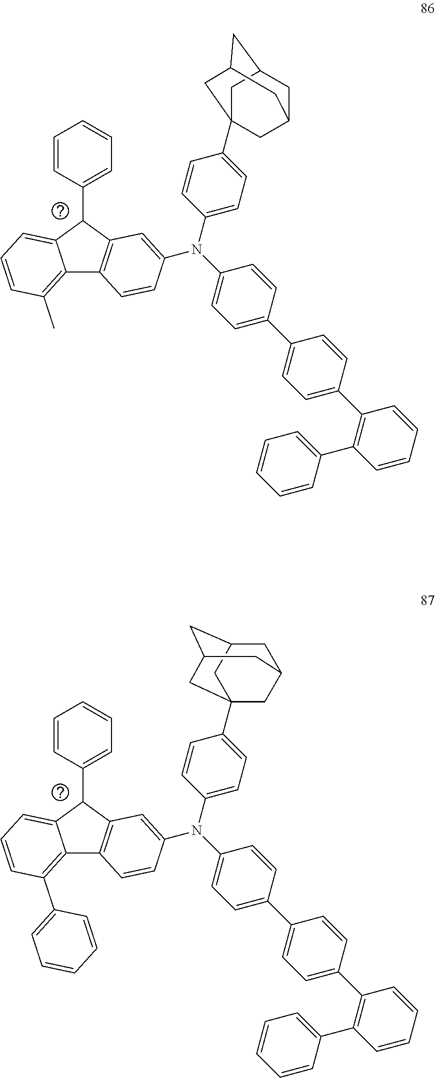 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2c1ccccc1 ; c1ccc(-c2ccccc2-c2ccc(-c3ccc(N(c4ccc(C56CC7CC(CC(C7)C5)C6)cc4)c4ccc5c(c4)C(c4ccccc4)c4cccc(-c6ccccc6)c4-5)cc3)cc2)cc1]

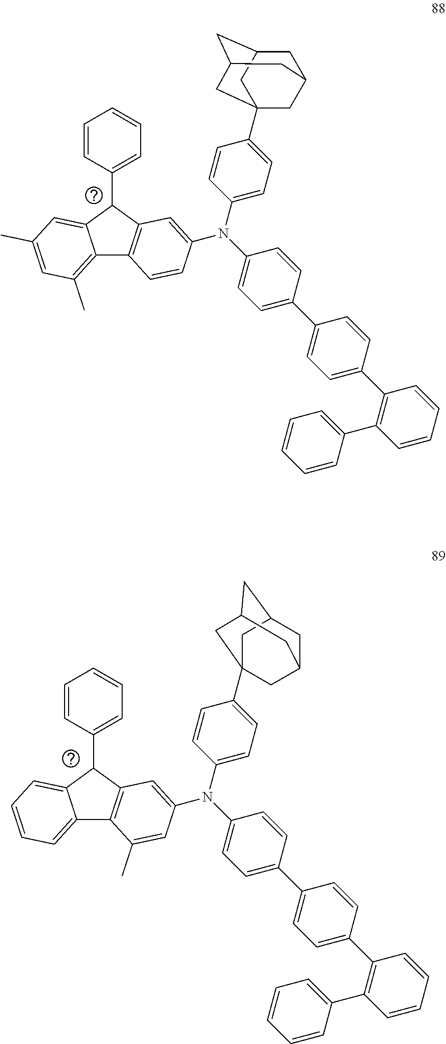 [SMILES: Cc1cc(C)c2c(c1)C(c1ccccc1)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2c1ccccc1]

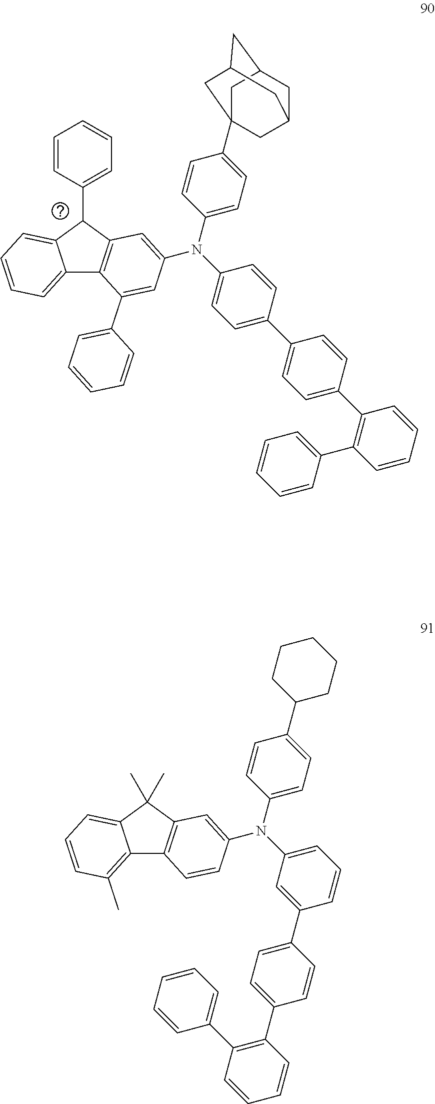 [SMILES: c1ccc(-c2ccccc2-c2ccc(-c3ccc(N(c4ccc(C56CC7CC(CC(C7)C5)C6)cc4)c4cc(-c5ccccc5)c5c(c4)C(c4ccccc4)c4ccccc4-5)cc3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2(C)C]

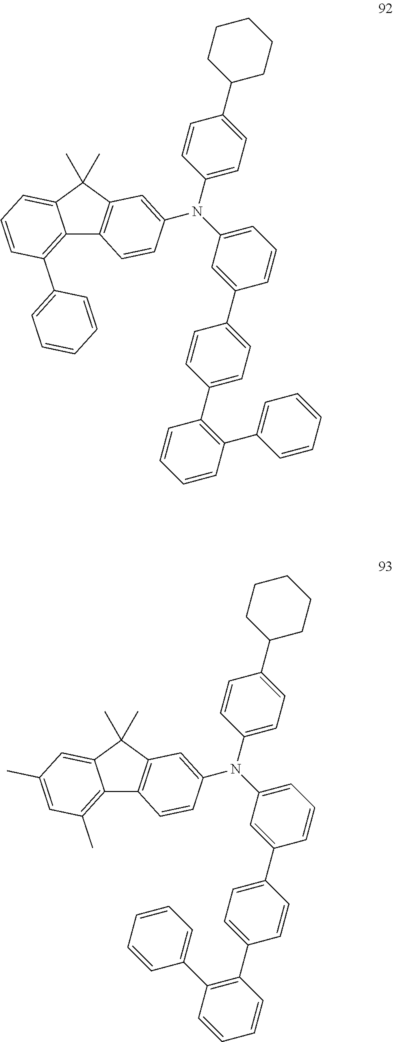 [SMILES: CC1(C)c2cc(N(c3ccc(C4CCCCC4)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C4CCCCC4)cc3)c3cccc(-c4ccc(-c5ccccc5-c5ccccc5)cc4)c3)ccc1-2]

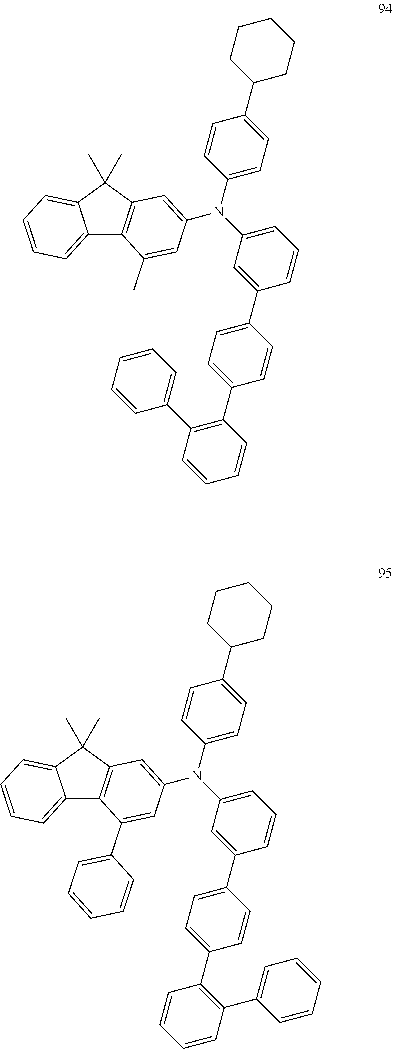 [SMILES: Cc1cc(N(c2ccc(C3CCCCC3)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CCCCC4)cc3)c3cccc(-c4ccc(-c5ccccc5-c5ccccc5)cc4)c3)cc21]

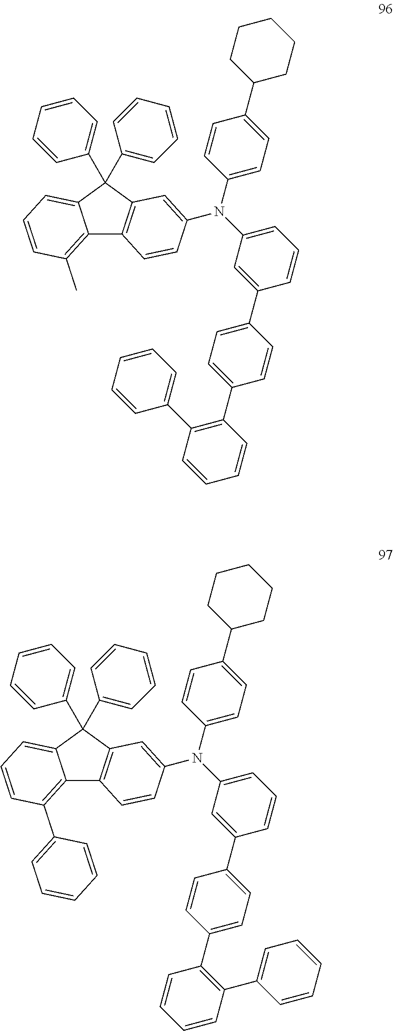 [SMILES: Cc1cccc2c1-c1ccc(N(c3ccc(C4CCCCC4)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CCCCC5)cc4)c4ccc5c(c4)C(c4ccccc4)(c4ccccc4)c4cccc(-c6ccccc6)c4-5)c3)cc2)cc1]

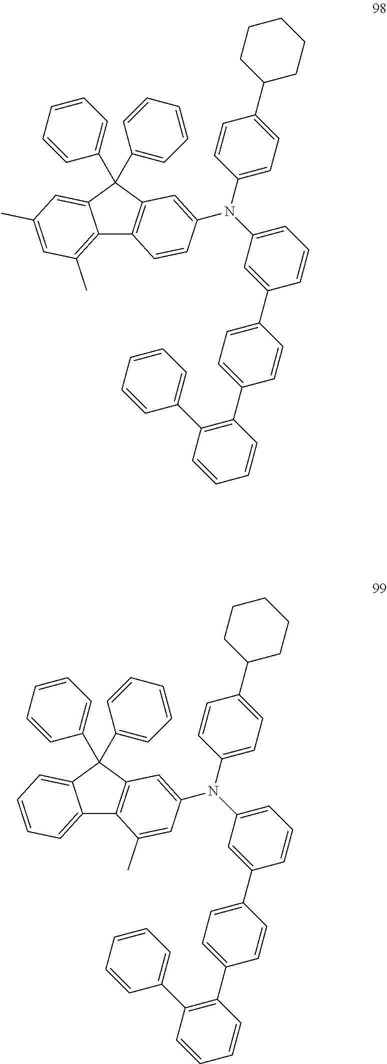 [SMILES: Cc1cc(C)c2c(c1)C(c1ccccc1)(c1ccccc1)c1cc(N(c3ccc(C4CCCCC4)cc3)c3cccc(-c4ccc(-c5ccccc5-c5ccccc5)cc4)c3)ccc1-2 ; Cc1cc(N(c2ccc(C3CCCCC3)cc2)c2cccc(-c3ccc(-c4ccccc4-c4ccccc4)cc3)c2)cc2c1-c1ccccc1C2(c1ccccc1)c1ccccc1]

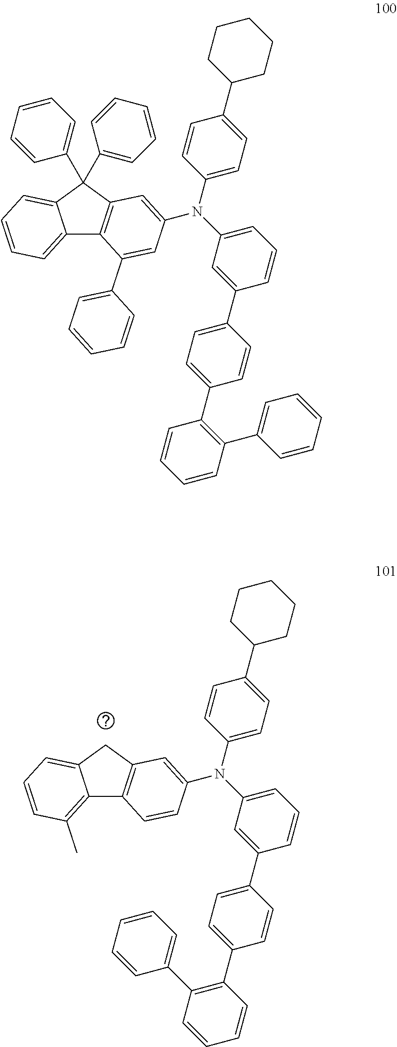 [SMILES: c1ccc(-c2ccccc2-c2ccc(-c3cccc(N(c4ccc(C5CCCCC5)cc4)c4cc(-c5ccccc5)c5c(c4)C(c4ccccc4)(c4ccccc4)c4ccccc4-5)c3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2]

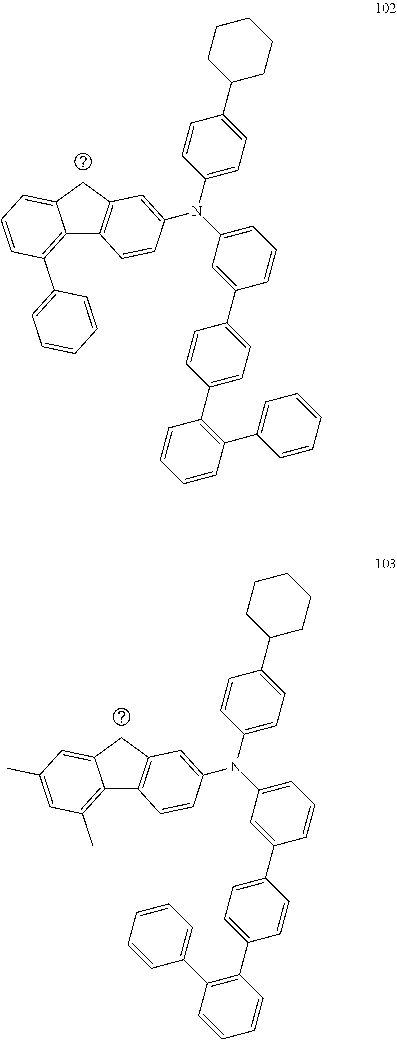 [SMILES: c1ccc(-c2ccccc2-c2ccc(-c3cccc(N(c4ccc(C5CCCCC5)cc4)c4ccc5c(c4)Cc4cccc(-c6ccccc6)c4-5)c3)cc2)cc1 ; Cc1cc(C)c2c(c1)Cc1cc(N(c3ccc(C4CCCCC4)cc3)c3cccc(-c4ccc(-c5ccccc5-c5ccccc5)cc4)c3)ccc1-2]

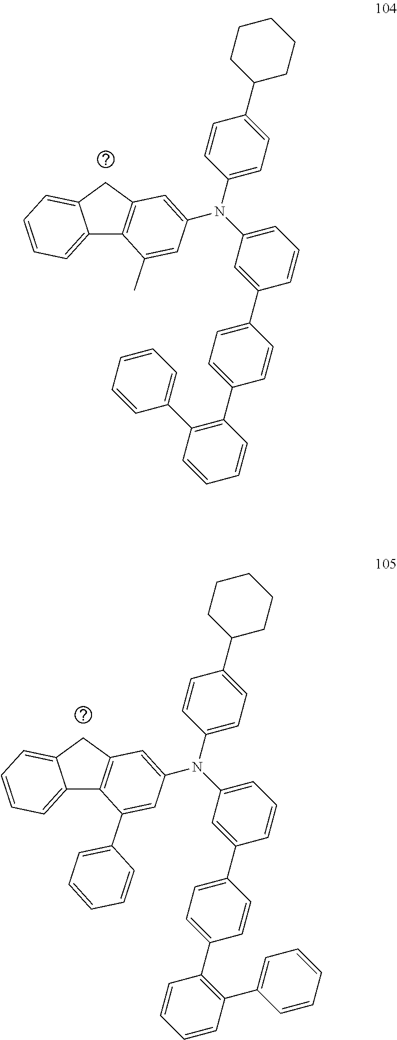 [SMILES: Cc1cc(N(c2ccc(C3CCCCC3)cc2)c2cccc(-c3ccc(-c4ccccc4-c4ccccc4)cc3)c2)cc2c1-c1ccccc1C2 ; c1ccc(-c2ccccc2-c2ccc(-c3cccc(N(c4ccc(C5CCCCC5)cc4)c4cc5c(c(-c6ccccc6)c4)-c4ccccc4C5)c3)cc2)cc1]

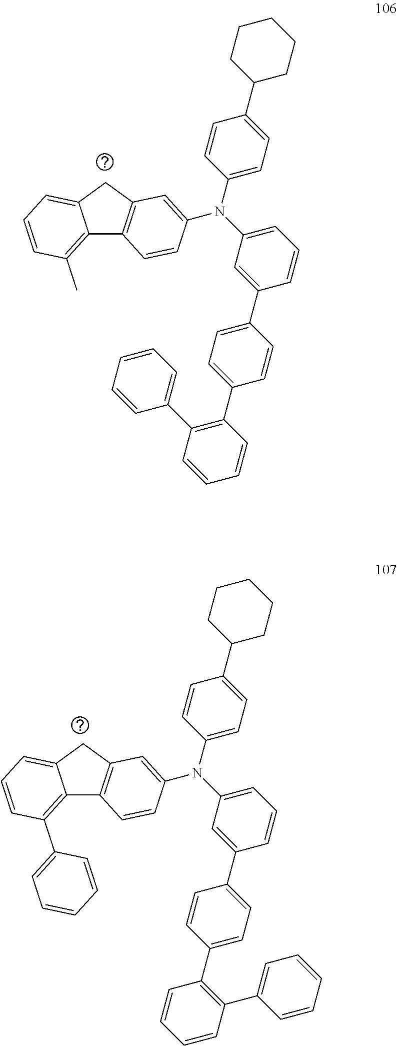 [SMILES: Cc1cccc2c1-c1ccc(N(c3ccc(C4CCCCC4)cc3)c3cccc(-c4ccc(-c5ccccc5-c5ccccc5)cc4)c3)cc1C2 ; c1ccc(-c2ccccc2-c2ccc(-c3cccc(N(c4ccc(C5CCCCC5)cc4)c4ccc5c(c4)Cc4cccc(-c6ccccc6)c4-5)c3)cc2)cc1]

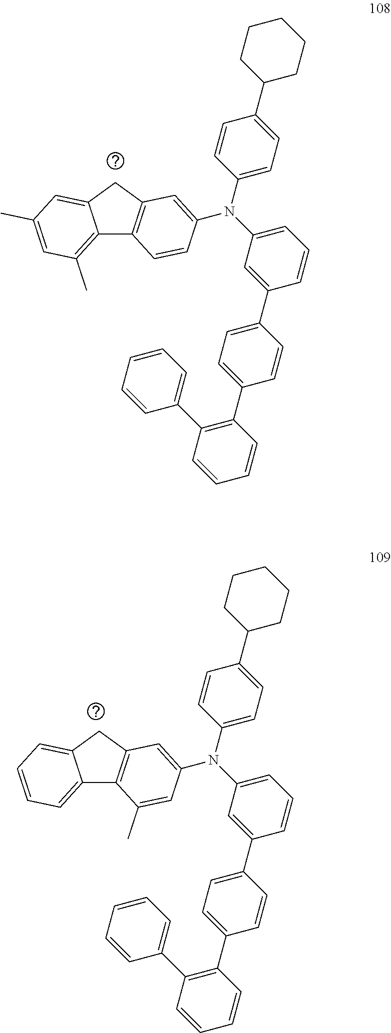 [SMILES: Cc1cc(C)c2c(c1)Cc1cc(N(c3ccc(C4CCCCC4)cc3)c3cccc(-c4ccc(-c5ccccc5-c5ccccc5)cc4)c3)ccc1-2 ; Cc1cc(N(c2ccc(C3CCCCC3)cc2)c2cccc(-c3ccc(-c4ccccc4-c4ccccc4)cc3)c2)cc2c1-c1ccccc1C2]

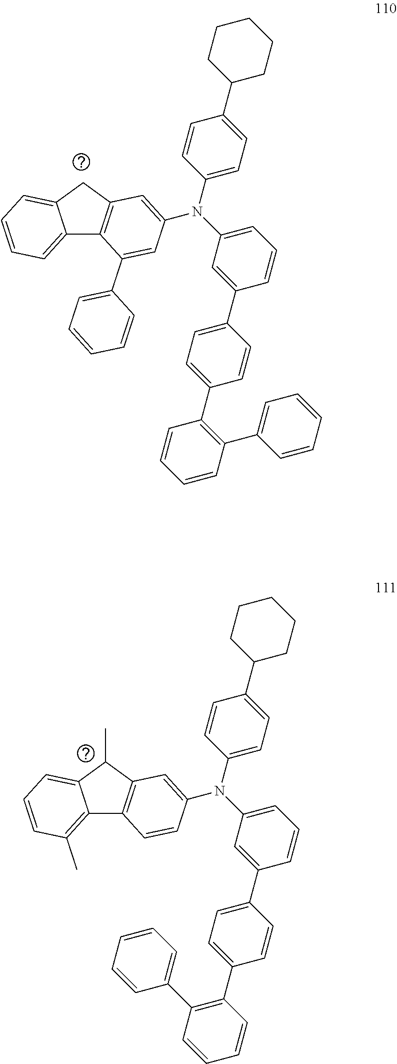 [SMILES: c1ccc(-c2ccccc2-c2ccc(-c3cccc(N(c4ccc(C5CCCCC5)cc4)c4cc5c(c(-c6ccccc6)c4)-c4ccccc4C5)c3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2C]

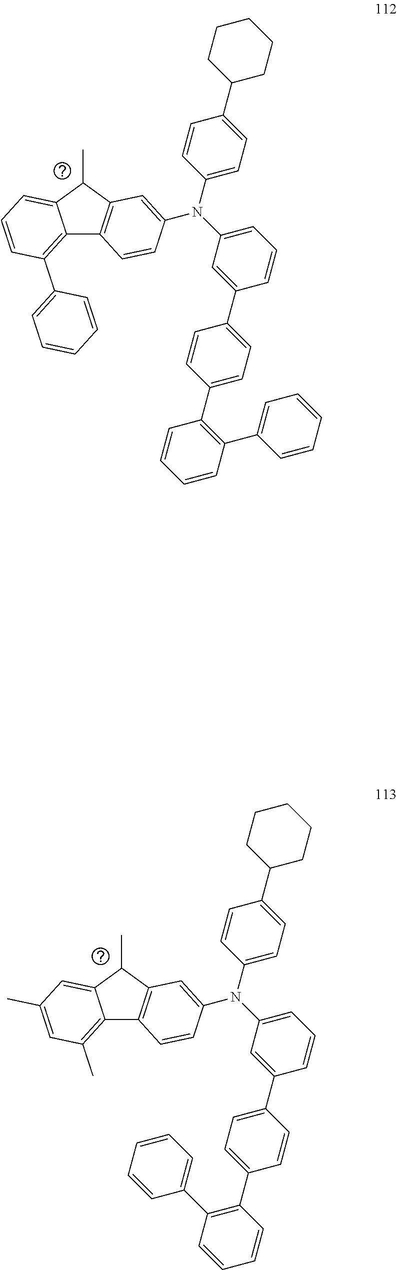 [SMILES: CC1c2cc(N(c3ccc(C4CCCCC4)cc3)c3cccc(-c4ccc(-c5ccccc5-c5ccccc5)cc4)c3)ccc2-c2c(-c3ccccc3)cccc21 ; Cc1cc(C)c2c(c1)C(C)c1cc(N(c3ccc(C4CCCCC4)cc3)c3cccc(-c4ccc(-c5ccccc5-c5ccccc5)cc4)c3)ccc1-2]

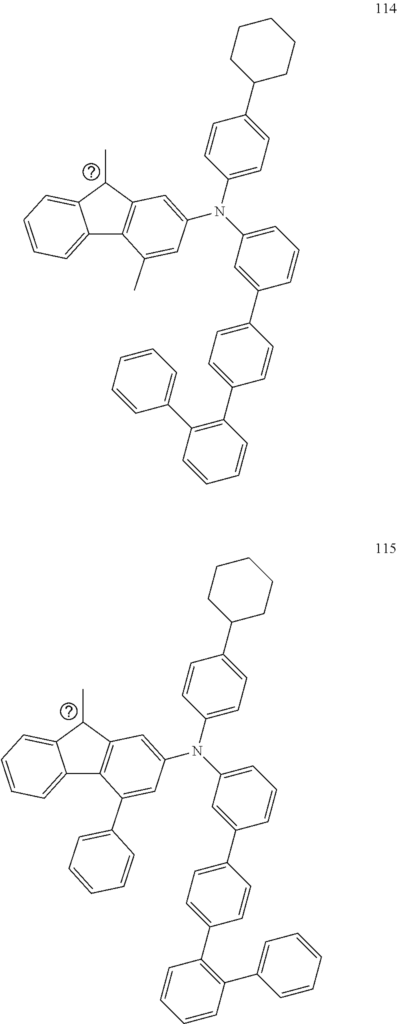 [SMILES: Cc1cc(N(c2ccc(C3CCCCC3)cc2)c2cccc(-c3ccc(-c4ccccc4-c4ccccc4)cc3)c2)cc2c1-c1ccccc1C2C ; CC1c2ccccc2-c2c(-c3ccccc3)cc(N(c3ccc(C4CCCCC4)cc3)c3cccc(-c4ccc(-c5ccccc5-c5ccccc5)cc4)c3)cc21]

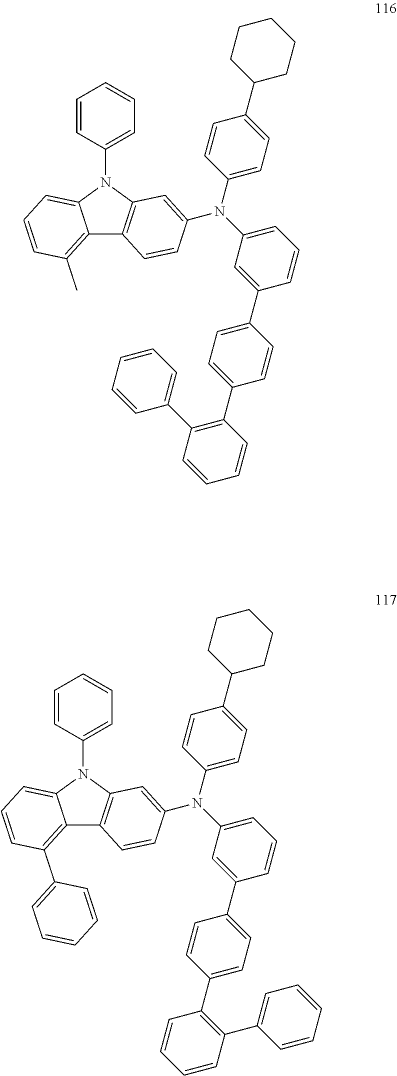 [SMILES: Cc1cccc2c1c1ccc(N(c3ccc(C4CCCCC4)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C5CCCCC5)cc4)c4ccc5c6c(-c7ccccc7)cccc6n(-c6ccccc6)c5c4)c3)cc2)cc1]

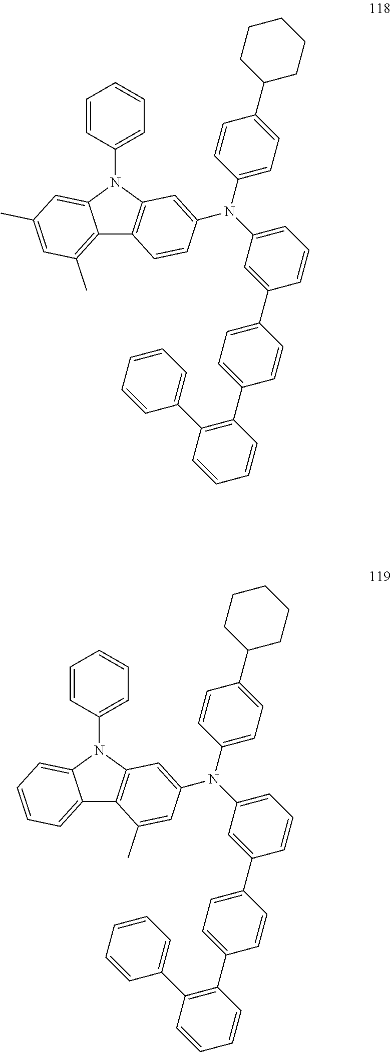 [SMILES: Cc1cc(C)c2c3ccc(N(c4ccc(C5CCCCC5)cc4)c4cccc(-c5ccc(-c6ccccc6-c6ccccc6)cc5)c4)cc3n(-c3ccccc3)c2c1 ; Cc1cc(N(c2ccc(C3CCCCC3)cc2)c2cccc(-c3ccc(-c4ccccc4-c4ccccc4)cc3)c2)cc2c1c1ccccc1n2-c1ccccc1]

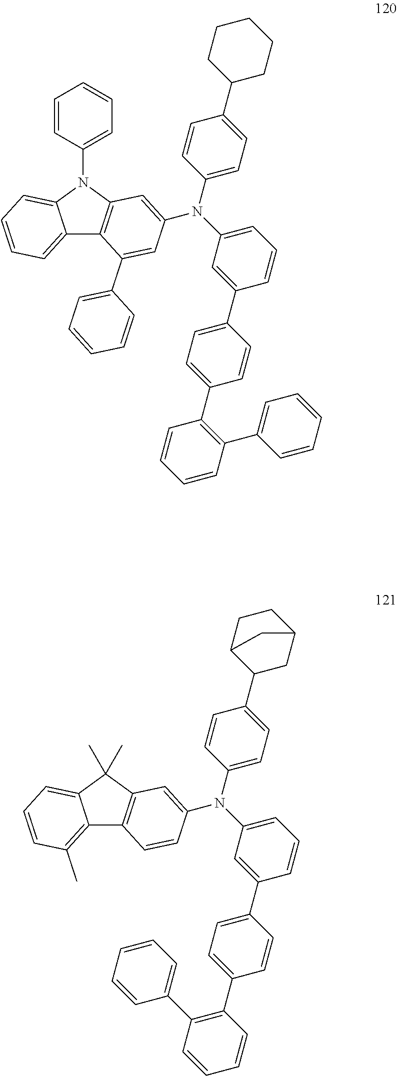 [SMILES: c1ccc(-c2ccccc2-c2ccc(-c3cccc(N(c4ccc(C5CCCCC5)cc4)c4cc(-c5ccccc5)c5c6ccccc6n(-c6ccccc6)c5c4)c3)cc2)cc1 ; Cc1cccc2c1-c1ccc(N(c3ccc(C4CC5CCC4C5)cc3)c3cccc(-c4ccc(-c5ccccc5-c5ccccc5)cc4)c3)cc1C2(C)C]

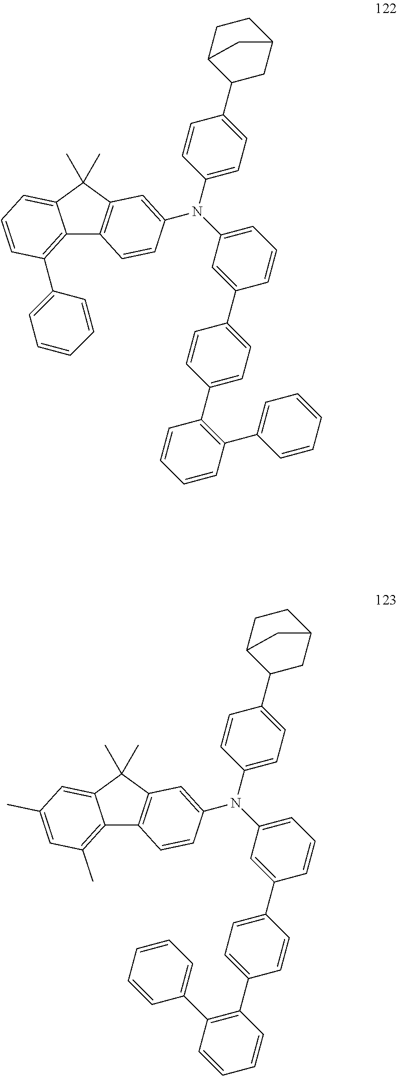 [SMILES: CC1(C)c2cc(N(c3ccc(C4CC5CCC4C5)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C4CC5CCC4C5)cc3)c3cccc(-c4ccc(-c5ccccc5-c5ccccc5)cc4)c3)ccc1-2]

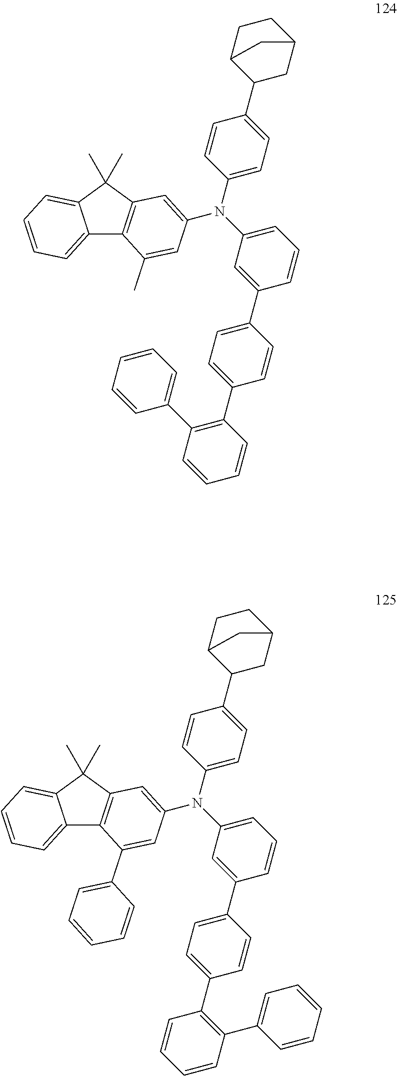 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CC5CCC4C5)cc3)c3cccc(-c4ccc(-c5ccccc5-c5ccccc5)cc4)c3)cc21]

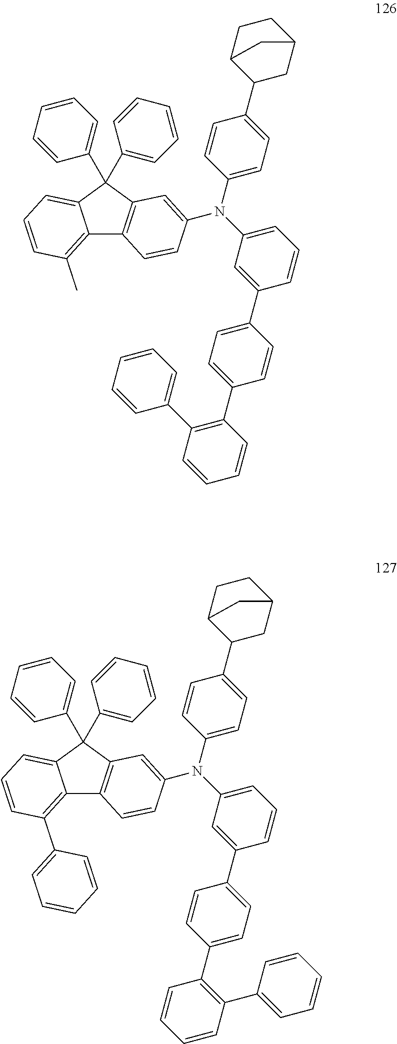 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CC6CCC5C6)cc4)c4ccc5c(c4)C(c4ccccc4)(c4ccccc4)c4cccc(-c6ccccc6)c4-5)c3)cc2)cc1]

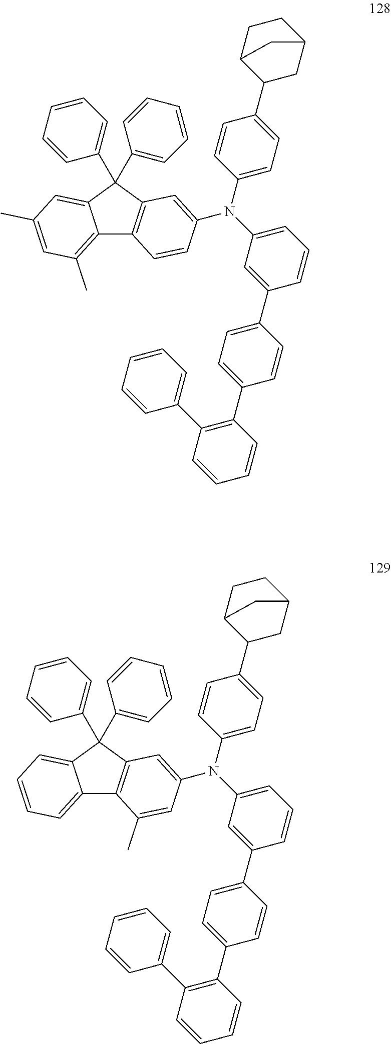 [SMILES: Cc1cc(C)c2c(c1)C(c1ccccc1)(c1ccccc1)c1cc(N(c3ccc(C4CC5CCC4C5)cc3)c3cccc(-c4ccc(-c5ccccc5-c5ccccc5)cc4)c3)ccc1-2 ; Cc1cc(N(c2ccc(C3CC4CCC3C4)cc2)c2cccc(-c3ccc(-c4ccccc4-c4ccccc4)cc3)c2)cc2c1-c1ccccc1C2(c1ccccc1)c1ccccc1]

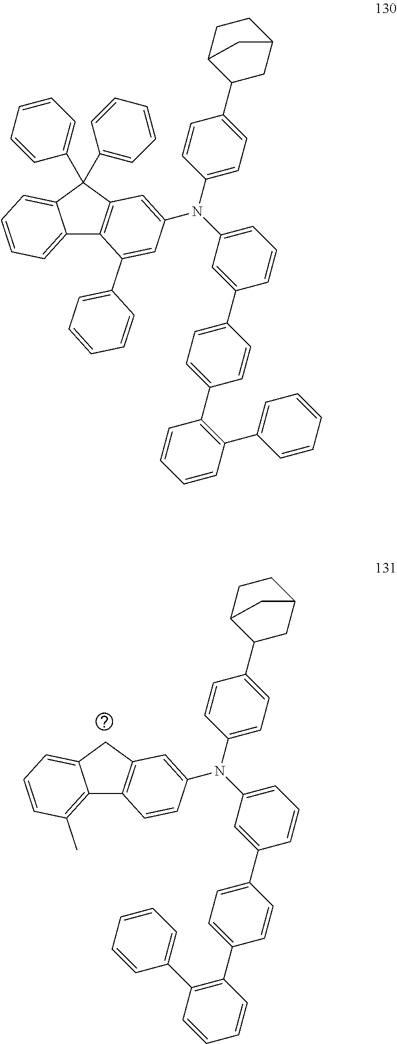 [SMILES: c1ccc(-c2ccccc2-c2ccc(-c3cccc(N(c4ccc(C5CC6CCC5C6)cc4)c4cc(-c5ccccc5)c5c(c4)C(c4ccccc4)(c4ccccc4)c4ccccc4-5)c3)cc2)cc1 ; Cc1cccc2c1-c1ccc(N(c3ccc(C4CC5CCC4C5)cc3)c3cccc(-c4ccc(-c5ccccc5-c5ccccc5)cc4)c3)cc1C2]

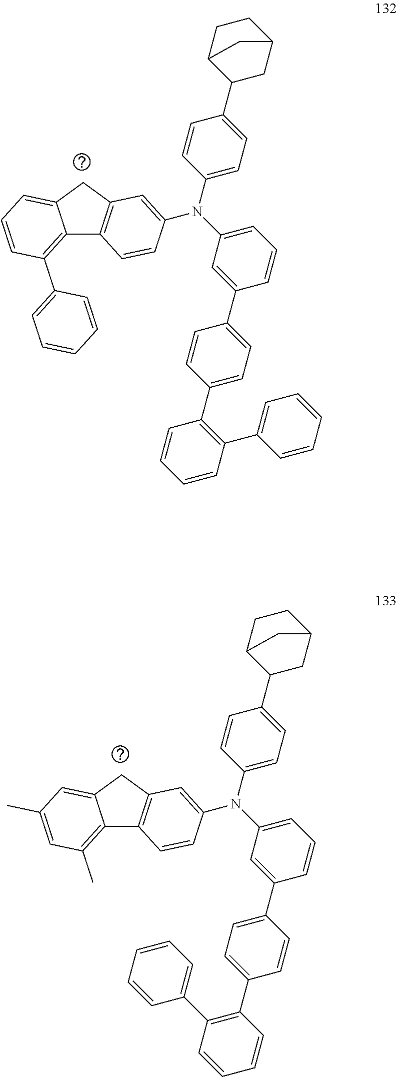 [SMILES: c1ccc(-c2ccccc2-c2ccc(-c3cccc(N(c4ccc(C5CC6CCC5C6)cc4)c4ccc5c(c4)Cc4cccc(-c6ccccc6)c4-5)c3)cc2)cc1 ; Cc1cc(C)c2c(c1)Cc1cc(N(c3ccc(C4CC5CCC4C5)cc3)c3cccc(-c4ccc(-c5ccccc5-c5ccccc5)cc4)c3)ccc1-2]

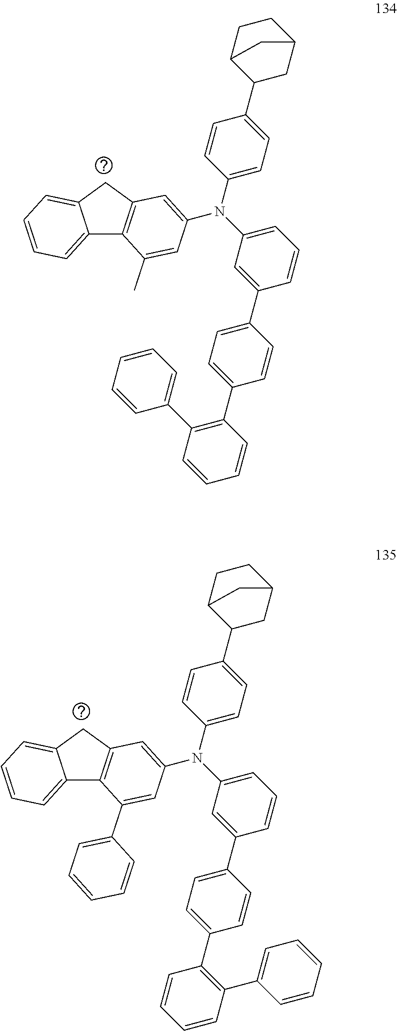 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2 ; c1ccc(-c2ccccc2-c2ccc(-c3cccc(N(c4ccc(C5CC6CCC5C6)cc4)c4cc5c(c(-c6ccccc6)c4)-c4ccccc4C5)c3)cc2)cc1]

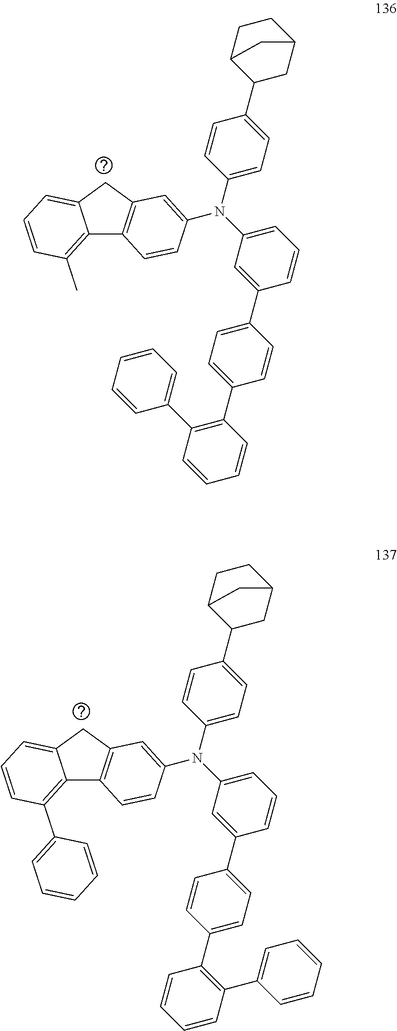 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2 ; c1ccc(-c2ccccc2-c2ccc(-c3cccc(N(c4ccc(C5CC6CCC5C6)cc4)c4ccc5c(c4)Cc4cccc(-c6ccccc6)c4-5)c3)cc2)cc1]

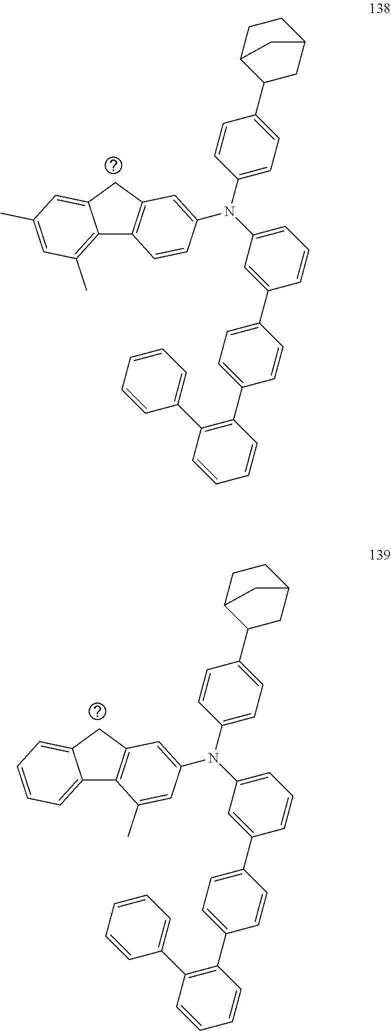 [SMILES: Cc1cc(C)c2c(c1)Cc1cc(N(c3ccc(C4CC5CCC4C5)cc3)c3cccc(-c4ccc(-c5ccccc5-c5ccccc5)cc4)c3)ccc1-2 ; Cc1cc(N(c2ccc(C3CC4CCC3C4)cc2)c2cccc(-c3ccc(-c4ccccc4-c4ccccc4)cc3)c2)cc2c1-c1ccccc1C2]

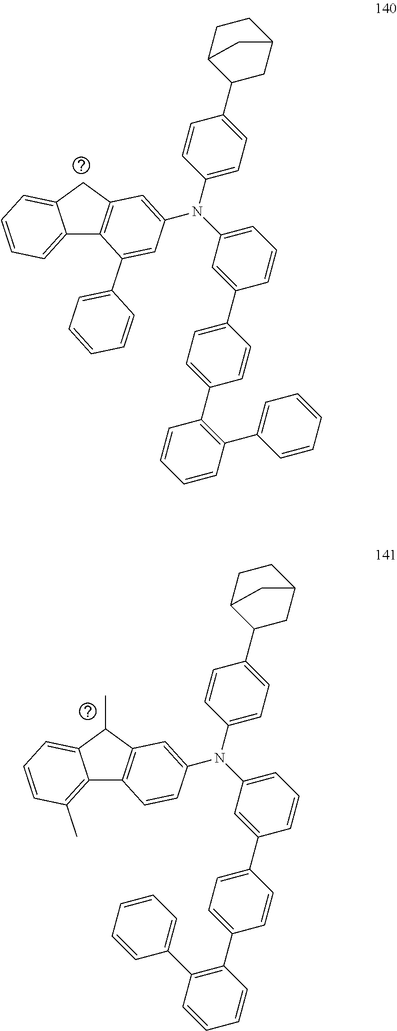 [SMILES: c1ccc(-c2ccccc2-c2ccc(-c3cccc(N(c4ccc(C5CC6CCC5C6)cc4)c4cc5c(c(-c6ccccc6)c4)-c4ccccc4C5)c3)cc2)cc1 ; Cc1cccc2c1-c1ccc(N(c3ccc(C4CC5CCC4C5)cc3)c3cccc(-c4ccc(-c5ccccc5-c5ccccc5)cc4)c3)cc1C2C]

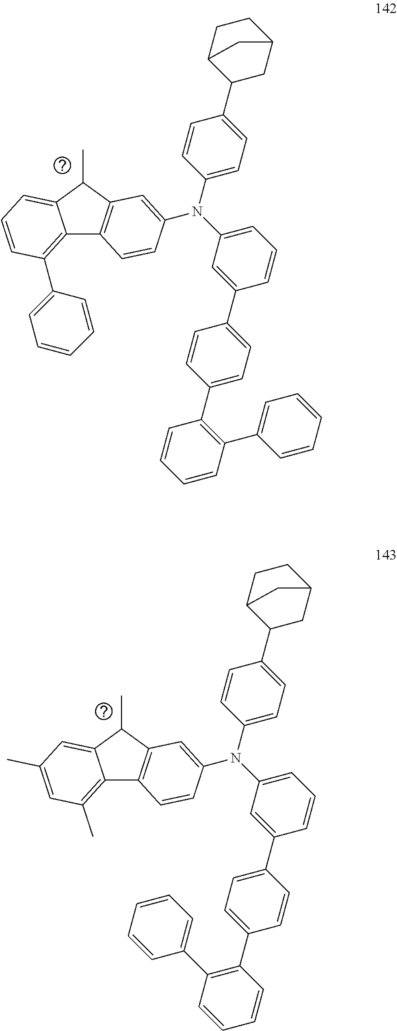 [SMILES: CC1c2cc(N(c3ccc(C4CC5CCC4C5)cc3)c3cccc(-c4ccc(-c5ccccc5-c5ccccc5)cc4)c3)ccc2-c2c(-c3ccccc3)cccc21 ; Cc1cc(C)c2c(c1)C(C)c1cc(N(c3ccc(C4CC5CCC4C5)cc3)c3cccc(-c4ccc(-c5ccccc5-c5ccccc5)cc4)c3)ccc1-2]

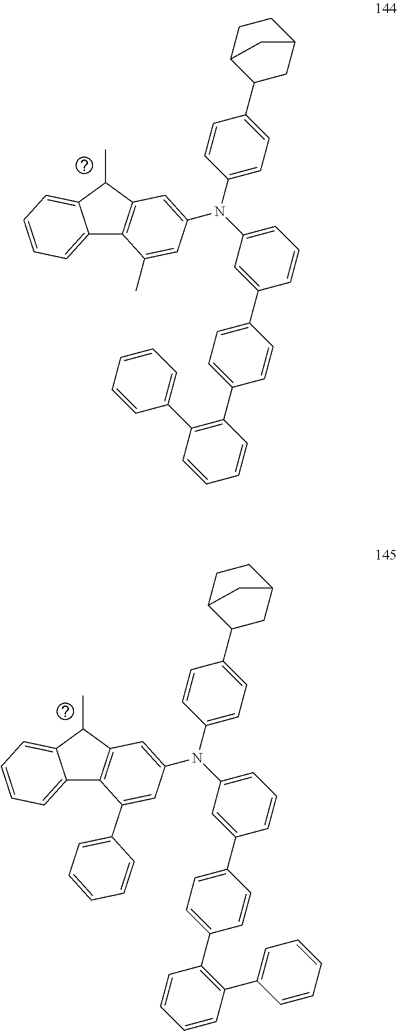 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2C ; CC1c2ccccc2-c2c(-c3ccccc3)cc(N(c3ccc(C4CC5CCC4C5)cc3)c3cccc(-c4ccc(-c5ccccc5-c5ccccc5)cc4)c3)cc21]

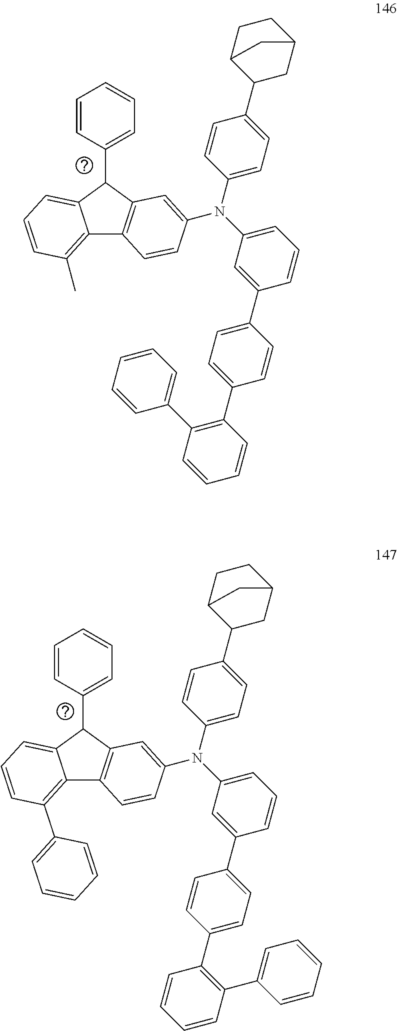 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2c1ccccc1 ; c1ccc(-c2ccccc2-c2ccc(-c3cccc(N(c4ccc(C5CC6CCC5C6)cc4)c4ccc5c(c4)C(c4ccccc4)c4cccc(-c6ccccc6)c4-5)c3)cc2)cc1]

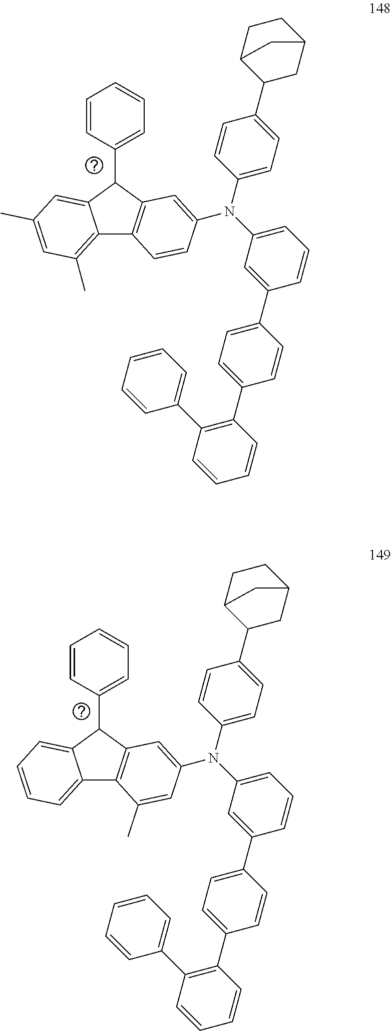 [SMILES: Cc1cc(C)c2c(c1)C(c1ccccc1)c1cc(N(c3ccc(C4CC5CCC4C5)cc3)c3cccc(-c4ccc(-c5ccccc5-c5ccccc5)cc4)c3)ccc1-2 ; Cc1cc(N(c2ccc(C3CC4CCC3C4)cc2)c2cccc(-c3ccc(-c4ccccc4-c4ccccc4)cc3)c2)cc2c1-c1ccccc1C2c1ccccc1]

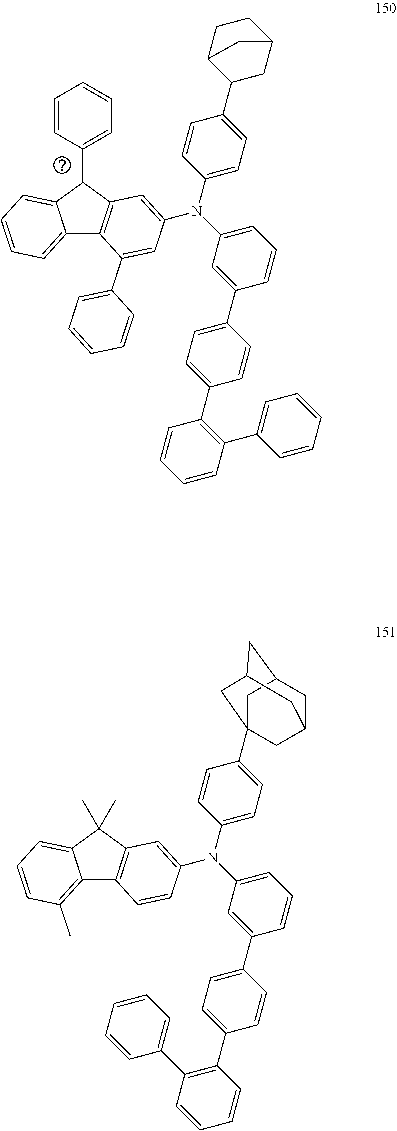 [SMILES: c1ccc(-c2ccccc2-c2ccc(-c3cccc(N(c4ccc(C5CC6CCC5C6)cc4)c4cc(-c5ccccc5)c5c(c4)C(c4ccccc4)c4ccccc4-5)c3)cc2)cc1 ; Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(C)C]

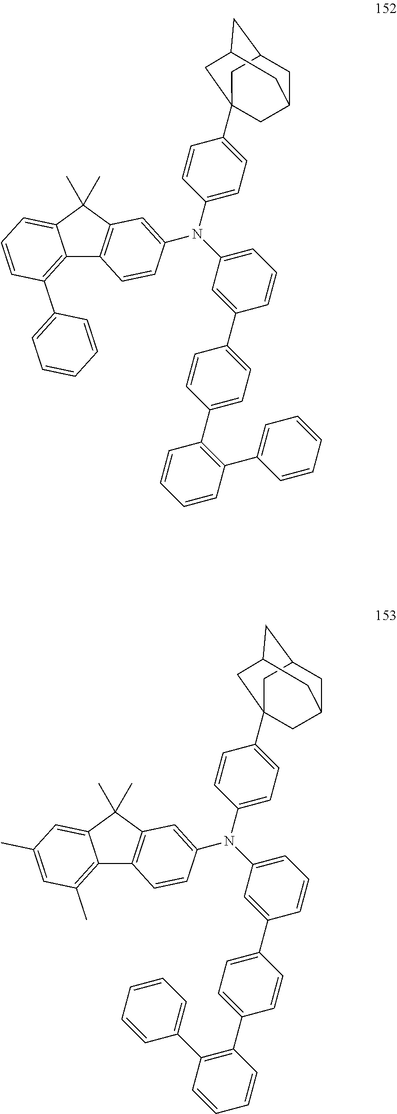 [SMILES: CC1(C)c2cc(N(c3ccc(C45CC6CC(CC(C6)C4)C5)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C45CC6CC(CC(C6)C4)C5)cc3)c3cccc(-c4ccc(-c5ccccc5-c5ccccc5)cc4)c3)ccc1-2]

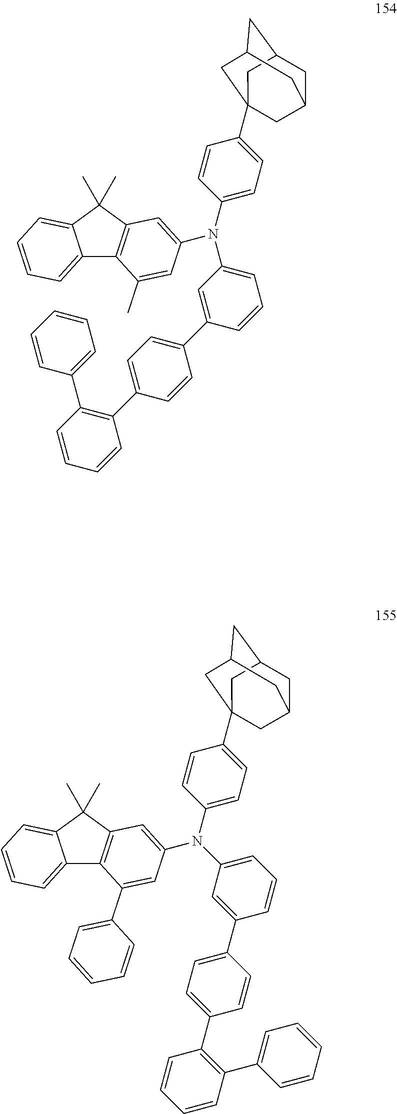 [SMILES: Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C45CC6CC(CC(C6)C4)C5)cc3)c3cccc(-c4ccc(-c5ccccc5-c5ccccc5)cc4)c3)cc21]

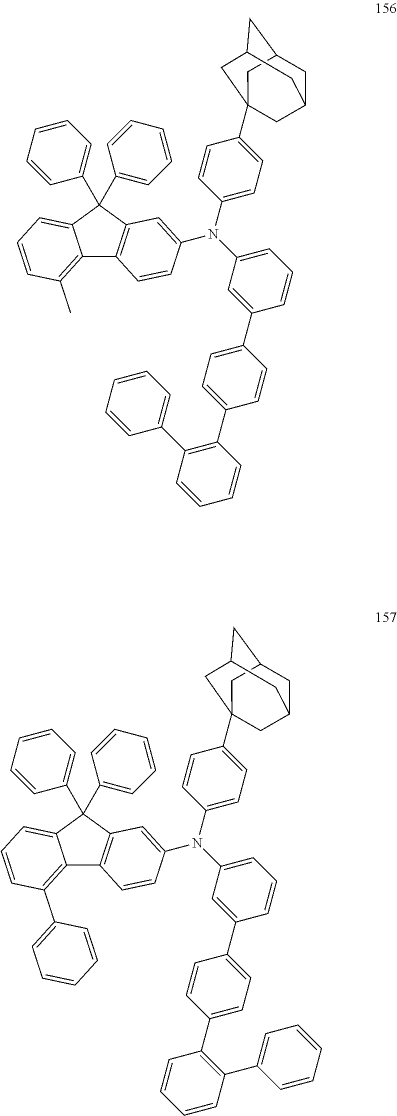 [SMILES: Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C56CC7CC(CC(C7)C5)C6)cc4)c4ccc5c(c4)C(c4ccccc4)(c4ccccc4)c4cccc(-c6ccccc6)c4-5)c3)cc2)cc1]

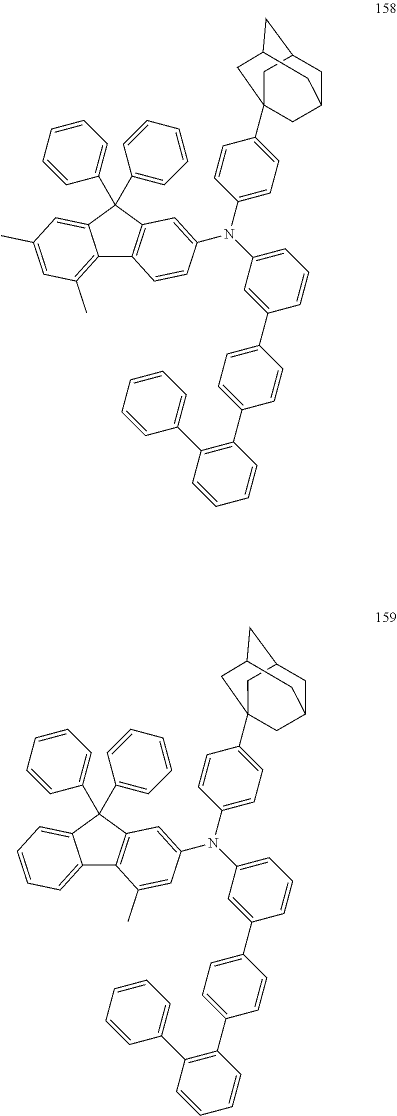 [SMILES: Cc1cc(C)c2c(c1)C(c1ccccc1)(c1ccccc1)c1cc(N(c3ccc(C45CC6CC(CC(C6)C4)C5)cc3)c3cccc(-c4ccc(-c5ccccc5-c5ccccc5)cc4)c3)ccc1-2 ; Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(c1ccccc1)c1ccccc1]

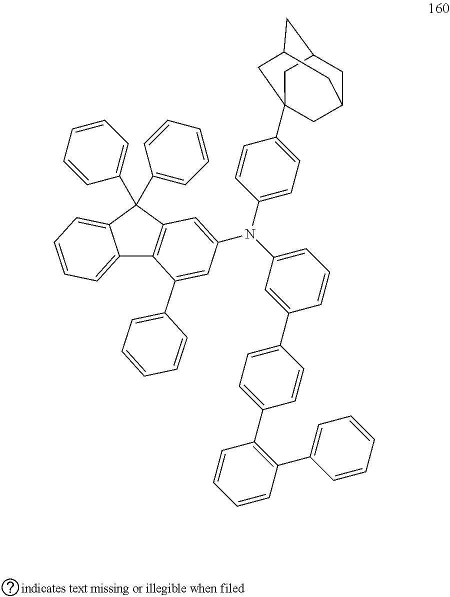 [SMILES: c1ccc(-c2ccccc2-c2ccc(-c3cccc(N(c4ccc(C56CC7CC(CC(C7)C5)C6)cc4)c4cc(-c5ccccc5)c5c(c4)C(c4ccccc4)(c4ccccc4)c4ccccc4-5)c3)cc2)cc1]

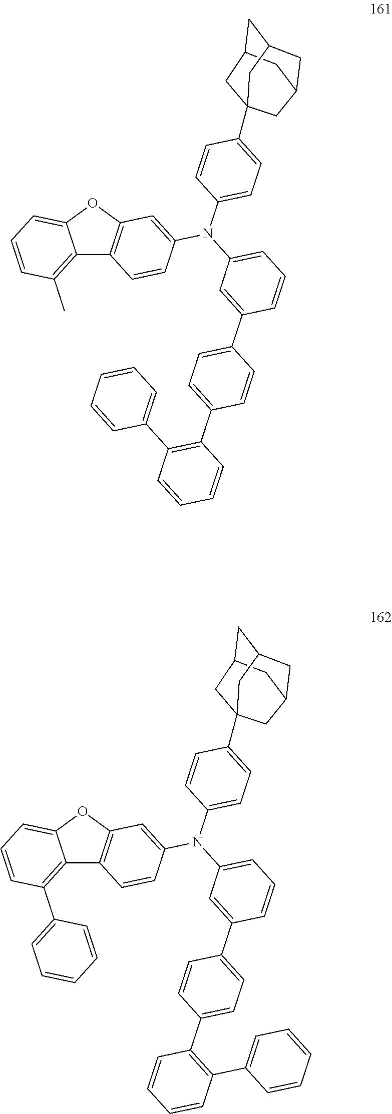 [SMILES: Cc1cccc2oc3cc(N(c4ccc(C56CC7CC(CC(C7)C5)C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C56CC7CC(CC(C7)C5)C6)cc4)c4ccc5c(c4)oc4cccc(-c6ccccc6)c45)c3)cc2)cc1]

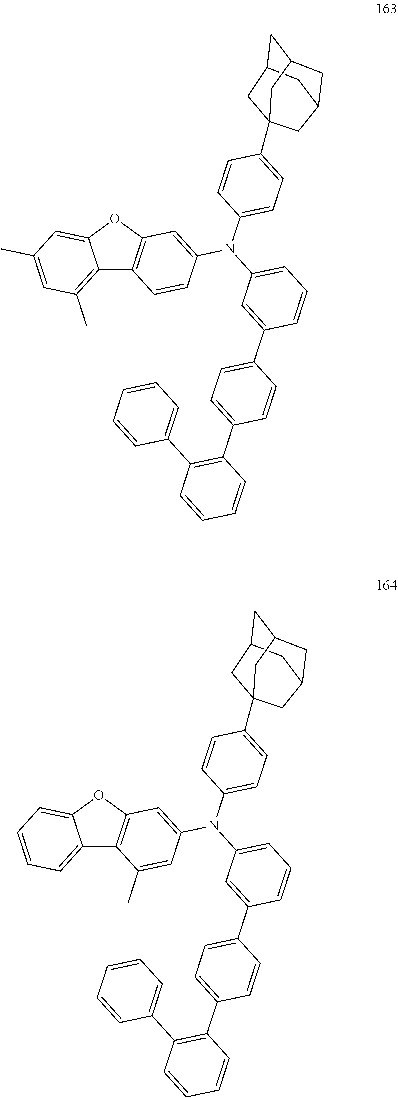 [SMILES: Cc1cc(C)c2c(c1)oc1cc(N(c3ccc(C45CC6CC(CC(C6)C4)C5)cc3)c3cccc(-c4ccc(-c5ccccc5-c5ccccc5)cc4)c3)ccc12 ; Cc1cc(N(c2ccc(C34CC5CC(CC(C5)C3)C4)cc2)c2cccc(-c3ccc(-c4ccccc4-c4ccccc4)cc3)c2)cc2oc3ccccc3c12]

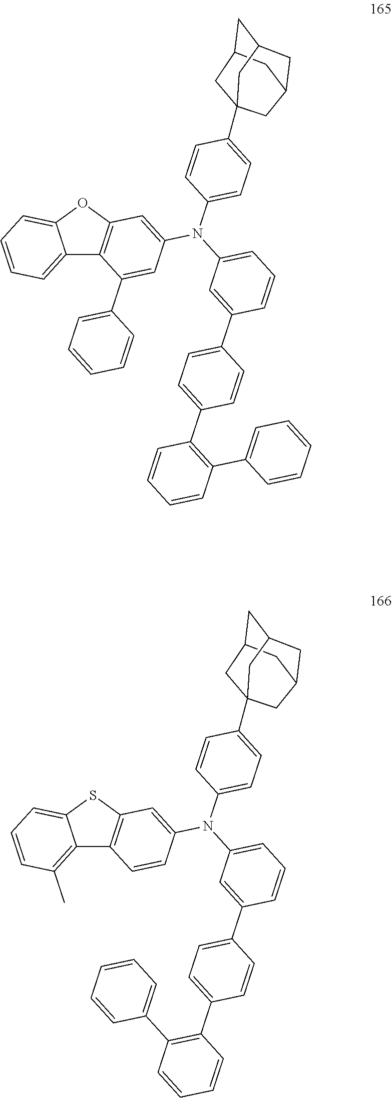 [SMILES: c1ccc(-c2ccccc2-c2ccc(-c3cccc(N(c4ccc(C56CC7CC(CC(C7)C5)C6)cc4)c4cc(-c5ccccc5)c5c(c4)oc4ccccc45)c3)cc2)cc1 ; Cc1cccc2sc3cc(N(c4ccc(C56CC7CC(CC(C7)C5)C6)cc4)c4cccc(-c5ccc(-c6ccccc6-c6ccccc6)cc5)c4)ccc3c12]

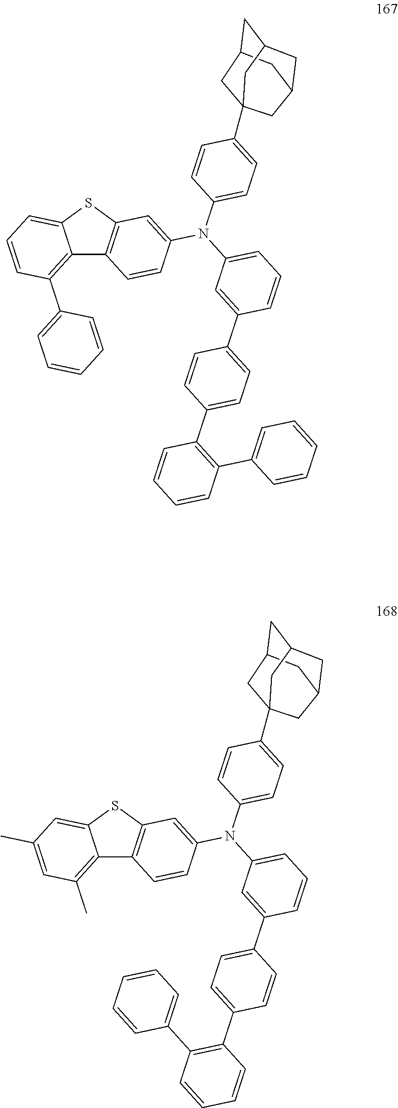 [SMILES: c1ccc(-c2ccccc2-c2ccc(-c3cccc(N(c4ccc(C56CC7CC(CC(C7)C5)C6)cc4)c4ccc5c(c4)sc4cccc(-c6ccccc6)c45)c3)cc2)cc1 ; Cc1cc(C)c2c(c1)sc1cc(N(c3ccc(C45CC6CC(CC(C6)C4)C5)cc3)c3cccc(-c4ccc(-c5ccccc5-c5ccccc5)cc4)c3)ccc12]

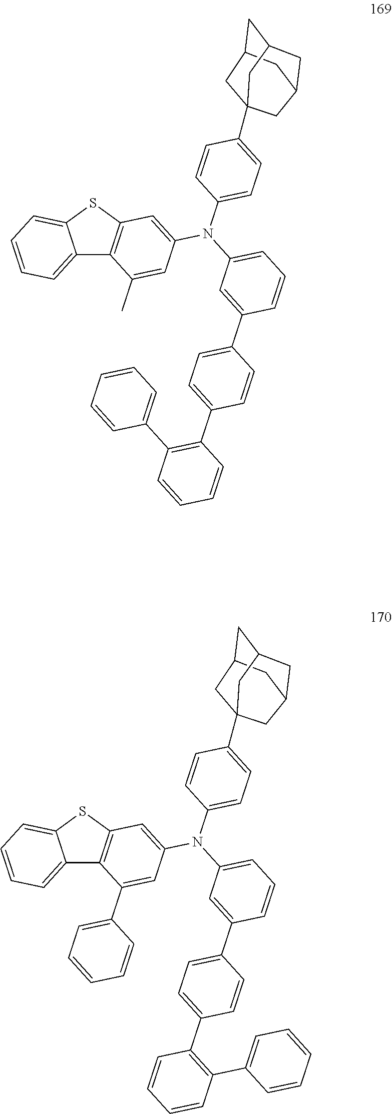 [SMILES: Cc1cc(N(c2ccc(C34CC5CC(CC(C5)C3)C4)cc2)c2cccc(-c3ccc(-c4ccccc4-c4ccccc4)cc3)c2)cc2sc3ccccc3c12 ; c1ccc(-c2ccccc2-c2ccc(-c3cccc(N(c4ccc(C56CC7CC(CC(C7)C5)C6)cc4)c4cc(-c5ccccc5)c5c(c4)sc4ccccc45)c3)cc2)cc1]

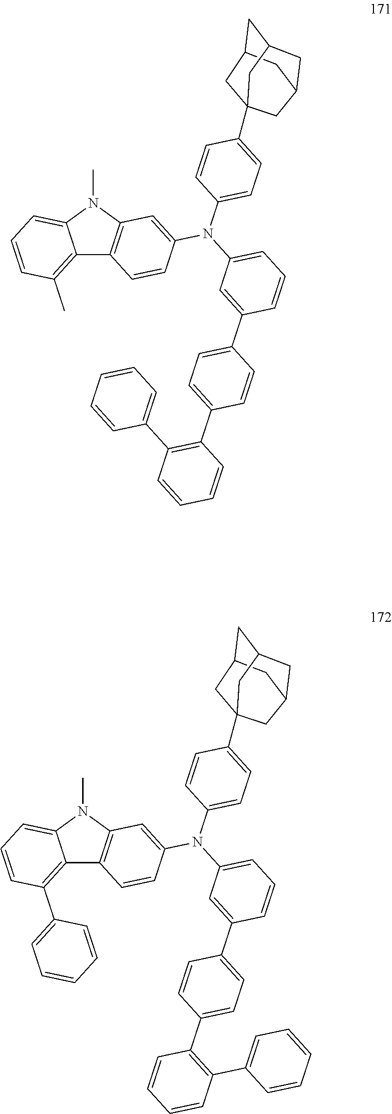 [SMILES: Cc1cccc2c1c1ccc(N(c3ccc(C45CC6CC(CC(C6)C4)C5)cc3)c3cccc(-c4ccc(-c5ccccc5-c5ccccc5)cc4)c3)cc1n2C ; Cn1c2cc(N(c3ccc(C45CC6CC(CC(C6)C4)C5)cc3)c3cccc(-c4ccc(-c5ccccc5-c5ccccc5)cc4)c3)ccc2c2c(-c3ccccc3)cccc21]

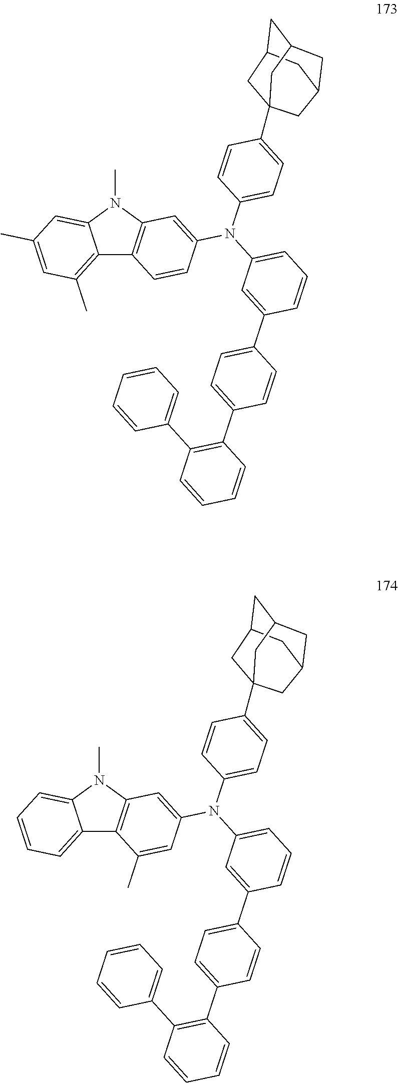 [SMILES: Cc1cc(C)c2c3ccc(N(c4ccc(C56CC7CC(CC(C7)C5)C6)cc4)c4cccc(-c5ccc(-c6ccccc6-c6ccccc6)cc5)c4)cc3n(C)c2c1 ; Cc1cc(N(c2ccc(C34CC5CC(CC(C5)C3)C4)cc2)c2cccc(-c3ccc(-c4ccccc4-c4ccccc4)cc3)c2)cc2c1c1ccccc1n2C]

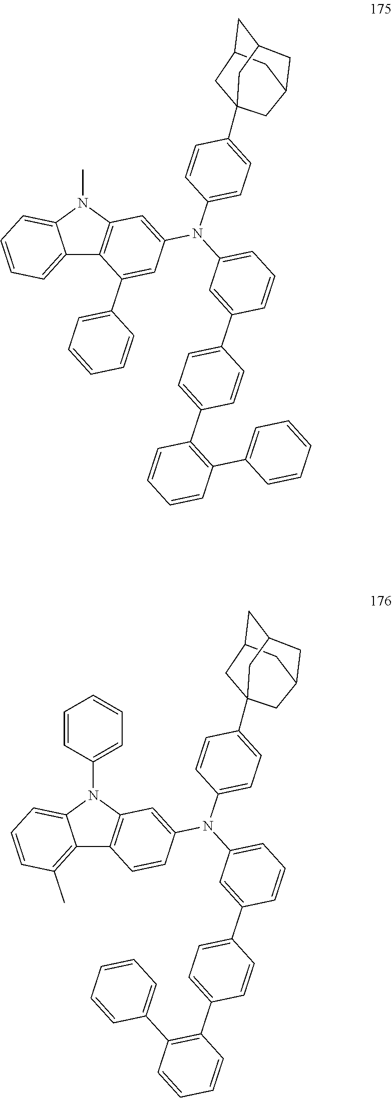 [SMILES: Cn1c2ccccc2c2c(-c3ccccc3)cc(N(c3ccc(C45CC6CC(CC(C6)C4)C5)cc3)c3cccc(-c4ccc(-c5ccccc5-c5ccccc5)cc4)c3)cc21 ; Cc1cccc2c1c1ccc(N(c3ccc(C45CC6CC(CC(C6)C4)C5)cc3)c3cccc(-c4ccc(-c5ccccc5-c5ccccc5)cc4)c3)cc1n2-c1ccccc1]

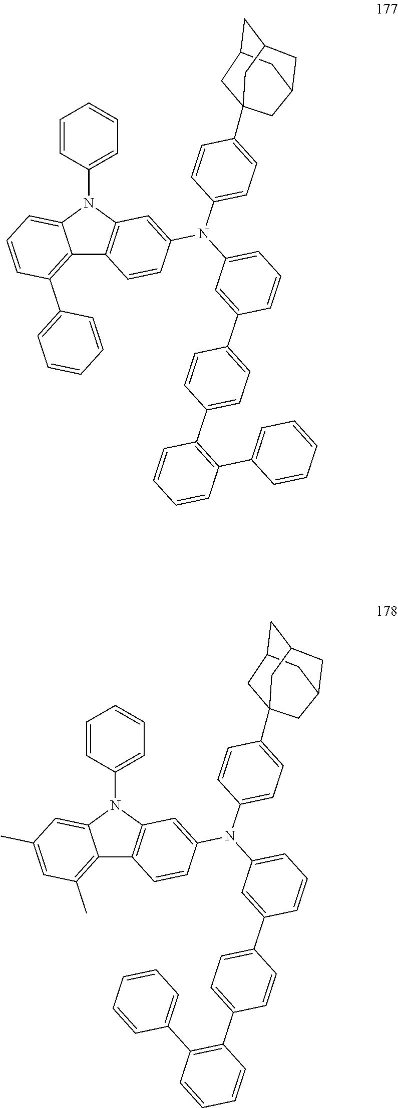 [SMILES: c1ccc(-c2ccccc2-c2ccc(-c3cccc(N(c4ccc(C56CC7CC(CC(C7)C5)C6)cc4)c4ccc5c6c(-c7ccccc7)cccc6n(-c6ccccc6)c5c4)c3)cc2)cc1 ; Cc1cc(C)c2c3ccc(N(c4ccc(C56CC7CC(CC(C7)C5)C6)cc4)c4cccc(-c5ccc(-c6ccccc6-c6ccccc6)cc5)c4)cc3n(-c3ccccc3)c2c1]

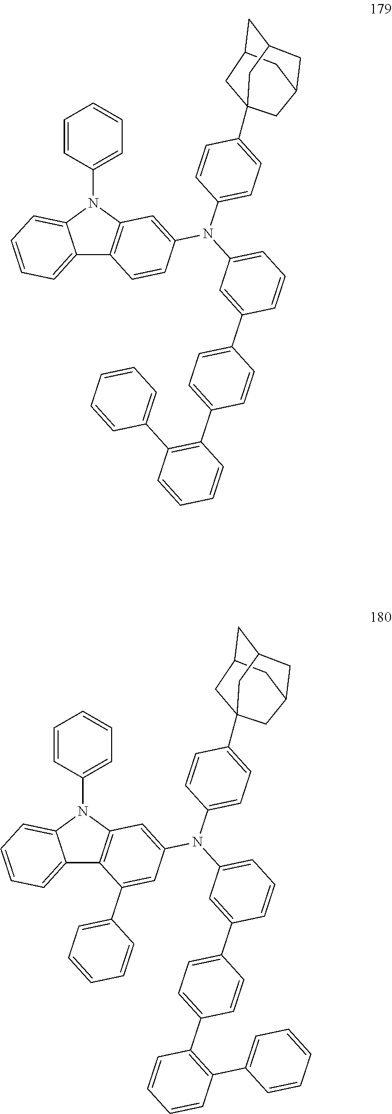 [SMILES: c1ccc(-c2ccccc2-c2ccc(-c3cccc(N(c4ccc(C56CC7CC(CC(C7)C5)C6)cc4)c4ccc5c6ccccc6n(-c6ccccc6)c5c4)c3)cc2)cc1 ; c1ccc(-c2ccccc2-c2ccc(-c3cccc(N(c4ccc(C56CC7CC(CC(C7)C5)C6)cc4)c4cc(-c5ccccc5)c5c6ccccc6n(-c6ccccc6)c5c4)c3)cc2)cc1]

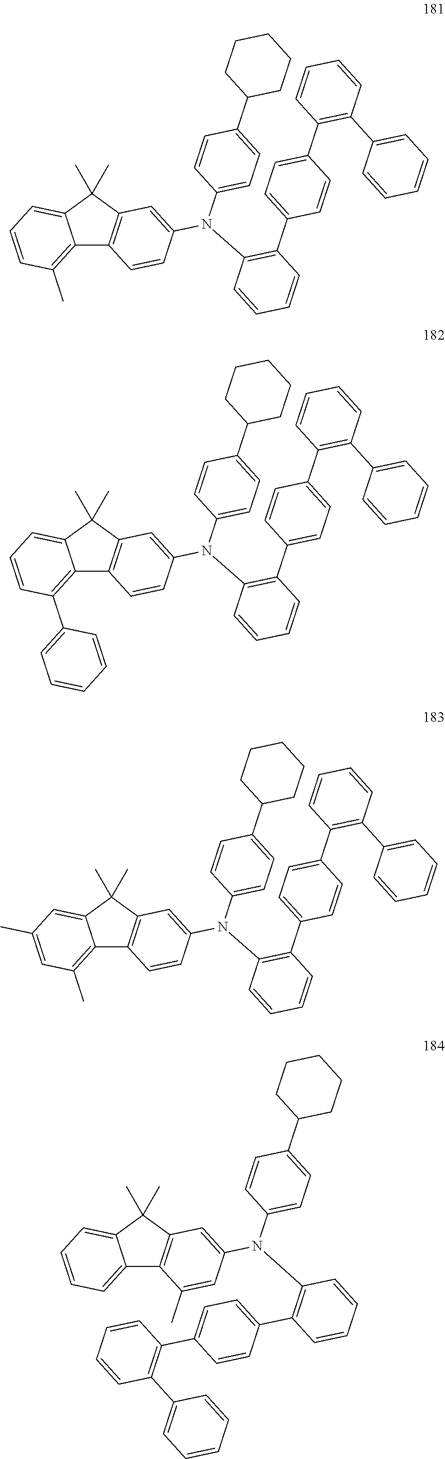 [SMILES: Cc1cccc2c1-c1ccc(N(c3ccc(C4CCCCC4)cc3)c3ccccc3-c3ccc(-c4ccccc4-c4ccccc4)cc3)cc1C2(C)C ; CC1(C)c2cc(N(c3ccc(C4CCCCC4)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CCCCC4)cc3)c3ccccc3-c3ccc(-c4ccccc4-c4ccccc4)cc3)ccc1-2 ; Cc1cc(N(c2ccc(C3CCCCC3)cc2)c2ccccc2-c2ccc(-c3ccccc3-c3ccccc3)cc2)cc2c1-c1ccccc1C2(C)C]

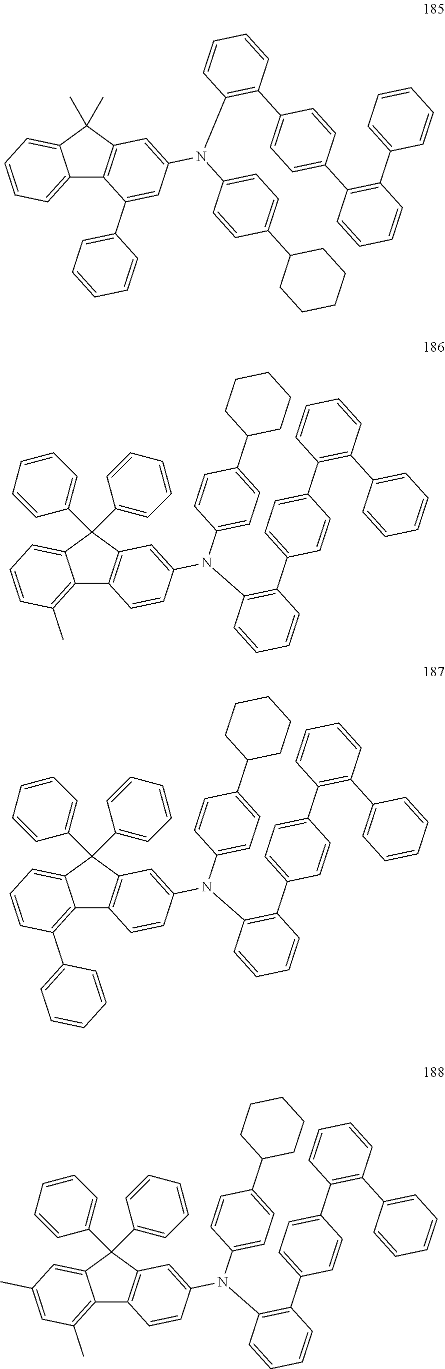

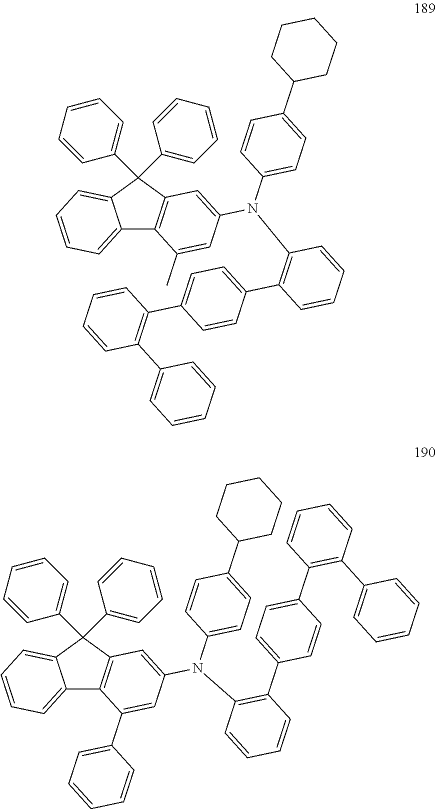 [SMILES: Cc1cc(N(c2ccc(C3CCCCC3)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C4CCCCC4)cc3)c3cc(-c4ccccc4)c4c(c3)C(c3ccccc3)(c3ccccc3)c3ccccc3-4)cc2)cc1]

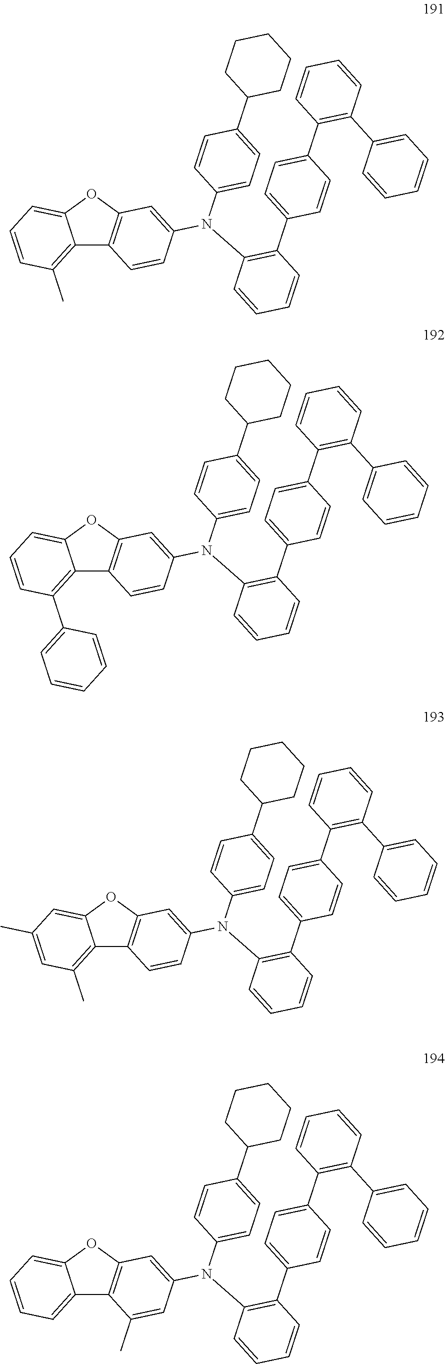 [SMILES: Cc1cccc2oc3cc(N(c4ccc(C5CCCCC5)cc4)c4ccccc4-c4ccc(-c5ccccc5-c5ccccc5)cc4)ccc3c12 ; c1ccc(-c2ccccc2-c2ccc(-c3ccccc3N(c3ccc(C4CCCCC4)cc3)c3ccc4c(c3)oc3cccc(-c5ccccc5)c34)cc2)cc1 ; Cc1cc(C)c2c(c1)oc1cc(N(c3ccc(C4CCCCC4)cc3)c3ccccc3-c3ccc(-c4ccccc4-c4ccccc4)cc3)ccc12 ; Cc1cc(N(c2ccc(C3CCCCC3)cc2)c2ccccc2-c2ccc(-c3ccccc3-c3ccccc3)cc2)cc2oc3ccccc3c12]

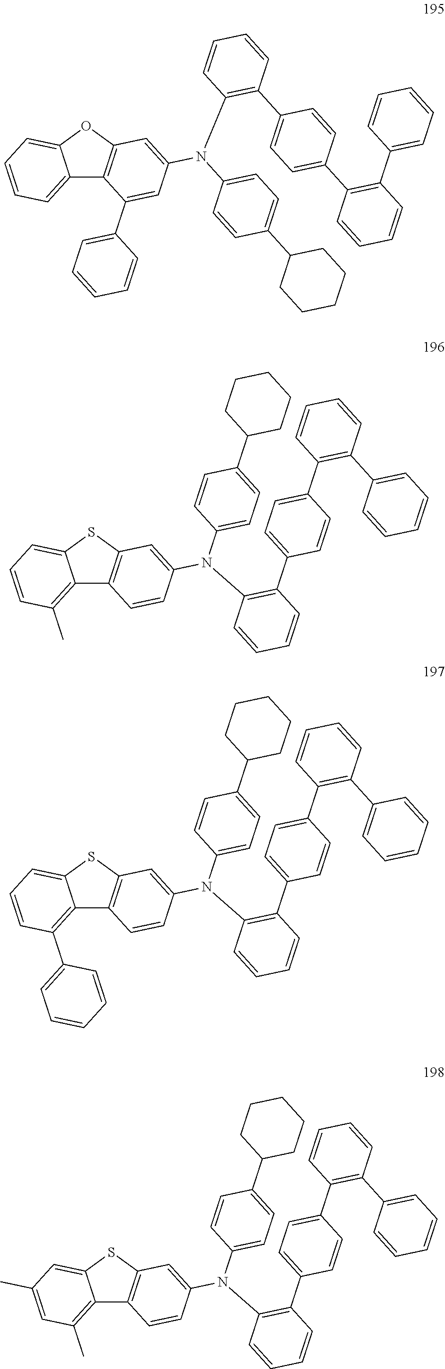

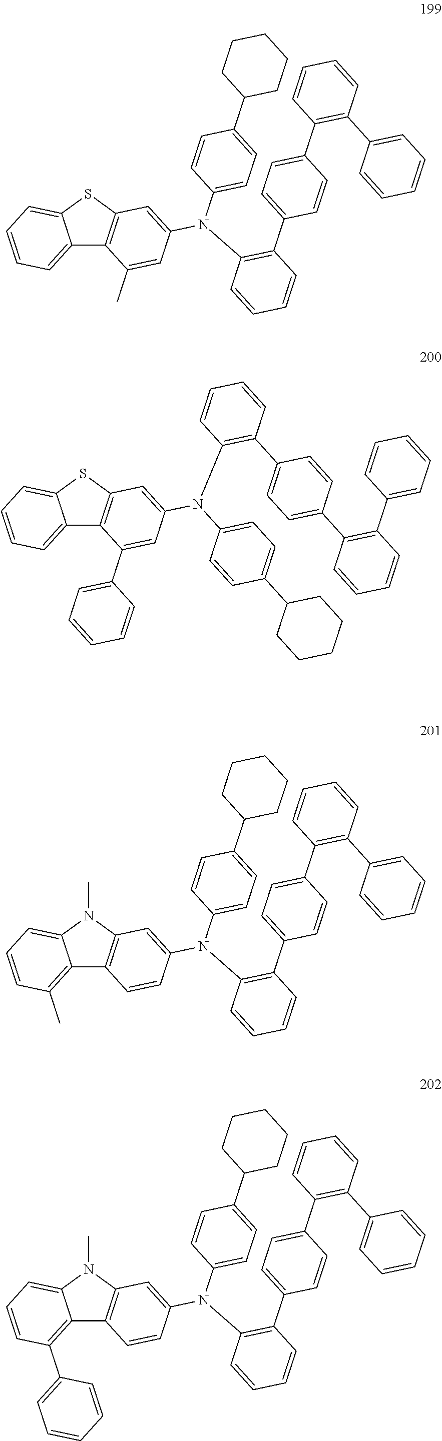 [SMILES: Cc1cc(N(c2ccc(C3CCCCC3)cc2)c2ccccc2-c2ccc(-c3ccccc3-c3ccccc3)cc2)cc2sc3ccccc3c12 ; c1ccc(-c2ccccc2-c2ccc(-c3ccccc3N(c3ccc(C4CCCCC4)cc3)c3cc(-c4ccccc4)c4c(c3)sc3ccccc34)cc2)cc1 ; Cc1cccc2c1c1ccc(N(c3ccc(C4CCCCC4)cc3)c3ccccc3-c3ccc(-c4ccccc4-c4ccccc4)cc3)cc1n2C ; Cn1c2cc(N(c3ccc(C4CCCCC4)cc3)c3ccccc3-c3ccc(-c4ccccc4-c4ccccc4)cc3)ccc2c2c(-c3ccccc3)cccc21]

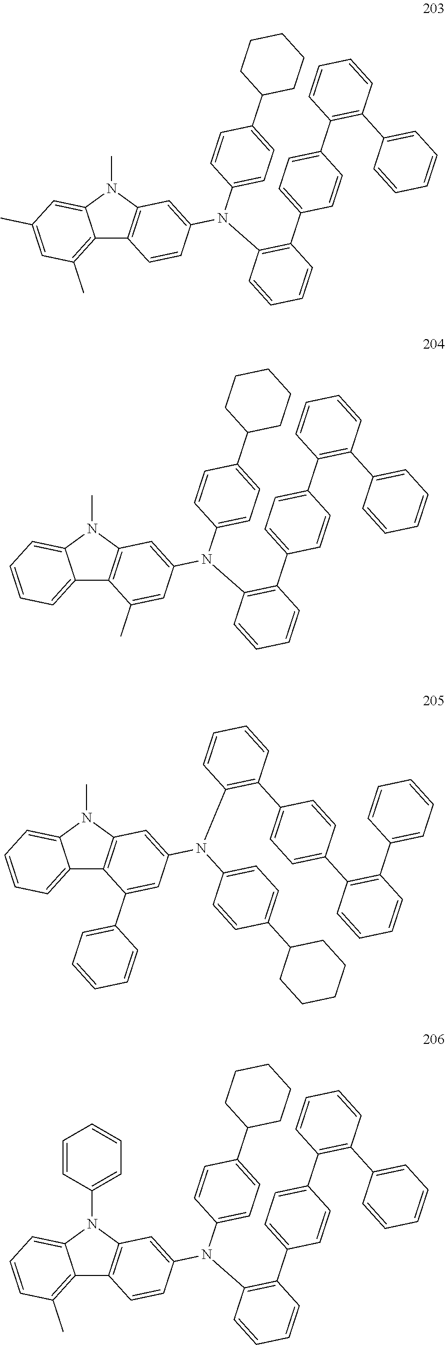 [SMILES: Cc1cc(C)c2c3ccc(N(c4ccc(C5CCCCC5)cc4)c4ccccc4-c4ccc(-c5ccccc5-c5ccccc5)cc4)cc3n(C)c2c1 ; Cc1cc(N(c2ccc(C3CCCCC3)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C4CCCCC4)cc3)c3ccccc3-c3ccc(-c4ccccc4-c4ccccc4)cc3)cc21 ; Cc1cccc2c1c1ccc(N(c3ccc(C4CCCCC4)cc3)c3ccccc3-c3ccc(-c4ccccc4-c4ccccc4)cc3)cc1n2-c1ccccc1]

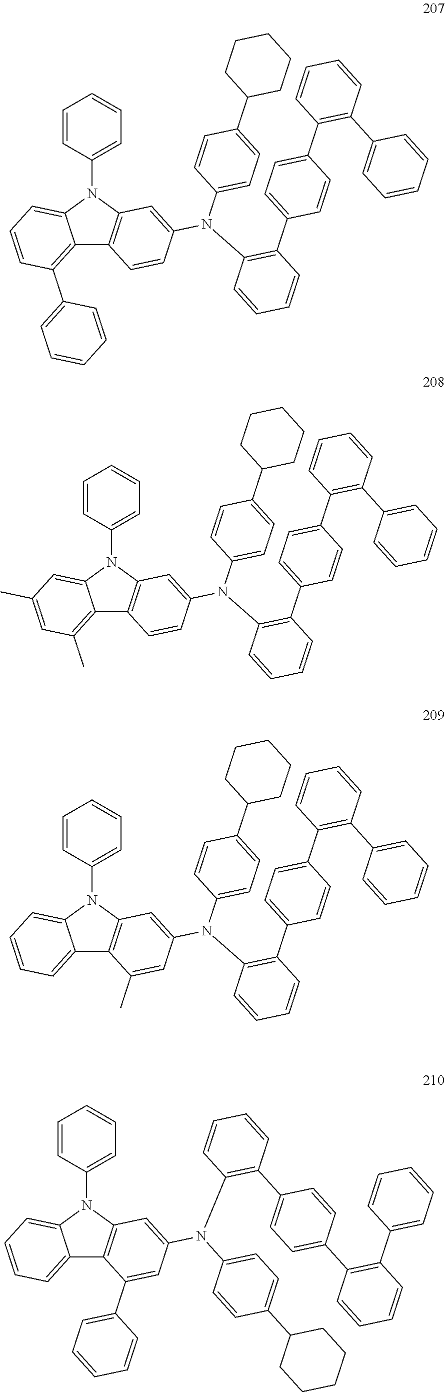 [SMILES: c1ccc(-c2ccccc2-c2ccc(-c3ccccc3N(c3ccc(C4CCCCC4)cc3)c3ccc4c5c(-c6ccccc6)cccc5n(-c5ccccc5)c4c3)cc2)cc1 ; Cc1cc(C)c2c3ccc(N(c4ccc(C5CCCCC5)cc4)c4ccccc4-c4ccc(-c5ccccc5-c5ccccc5)cc4)cc3n(-c3ccccc3)c2c1 ; Cc1cc(N(c2ccc(C3CCCCC3)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CCCCC4)cc3)c3cc(-c4ccccc4)c4c5ccccc5n(-c5ccccc5)c4c3)cc2)cc1]

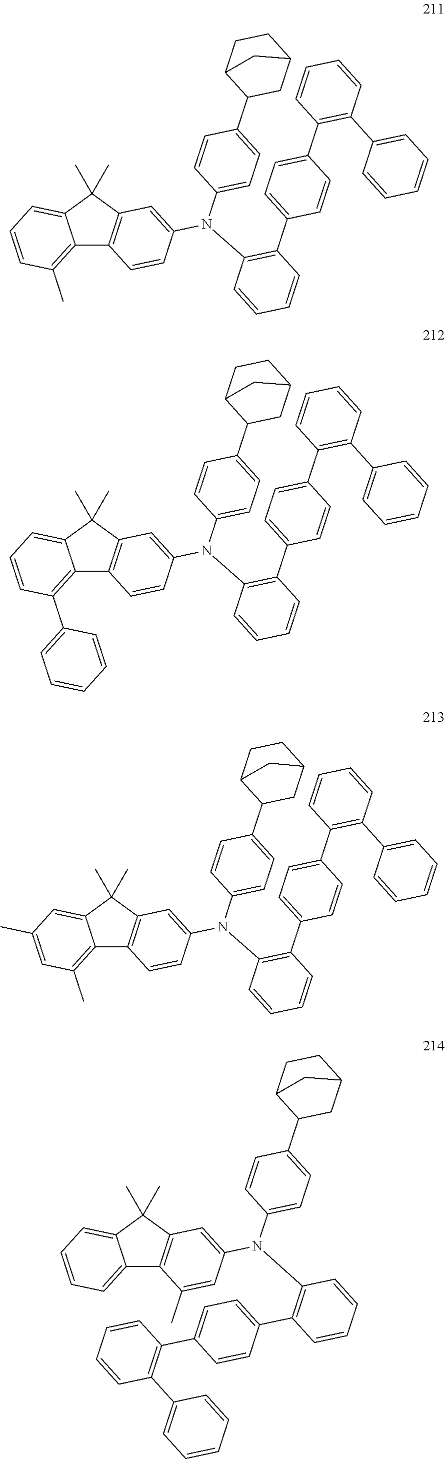 [SMILES: Cc1cccc2c1-c1ccc(N(c3ccc(C4CC5CCC4C5)cc3)c3ccccc3-c3ccc(-c4ccccc4-c4ccccc4)cc3)cc1C2(C)C ; CC1(C)c2cc(N(c3ccc(C4CC5CCC4C5)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CC5CCC4C5)cc3)c3ccccc3-c3ccc(-c4ccccc4-c4ccccc4)cc3)ccc1-2 ; Cc1cc(N(c2ccc(C3CC4CCC3C4)cc2)c2ccccc2-c2ccc(-c3ccccc3-c3ccccc3)cc2)cc2c1-c1ccccc1C2(C)C]

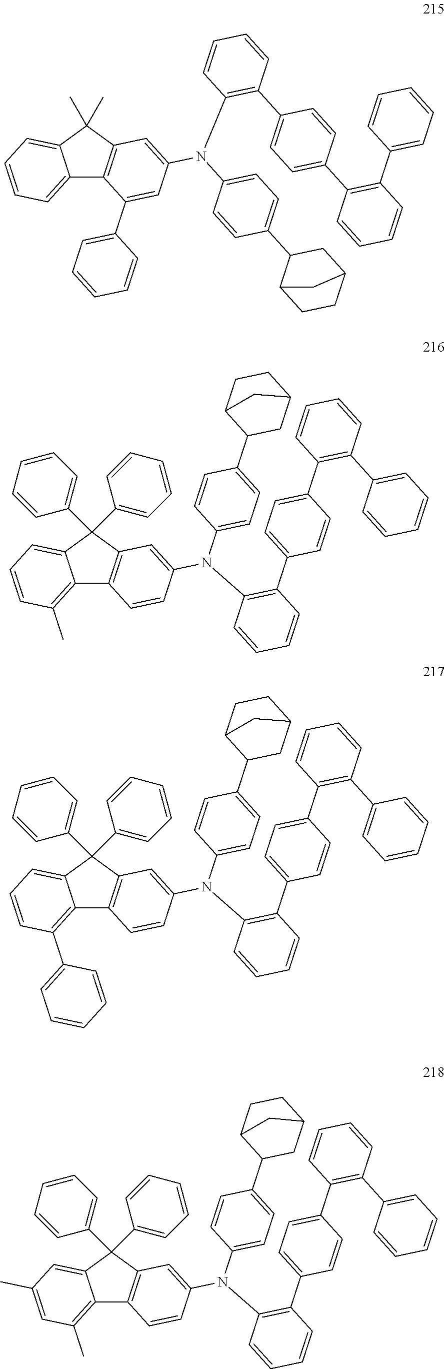

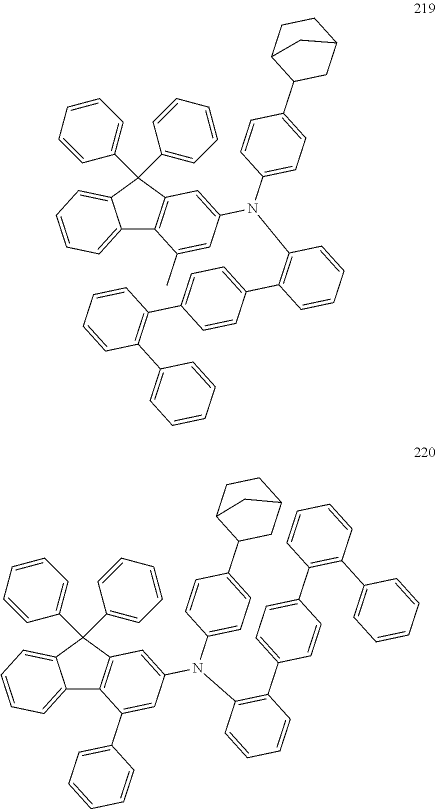 [SMILES: Cc1cc(N(c2ccc(C3CC4CCC3C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C4CC5CCC4C5)cc3)c3cc(-c4ccccc4)c4c(c3)C(c3ccccc3)(c3ccccc3)c3ccccc3-4)cc2)cc1]

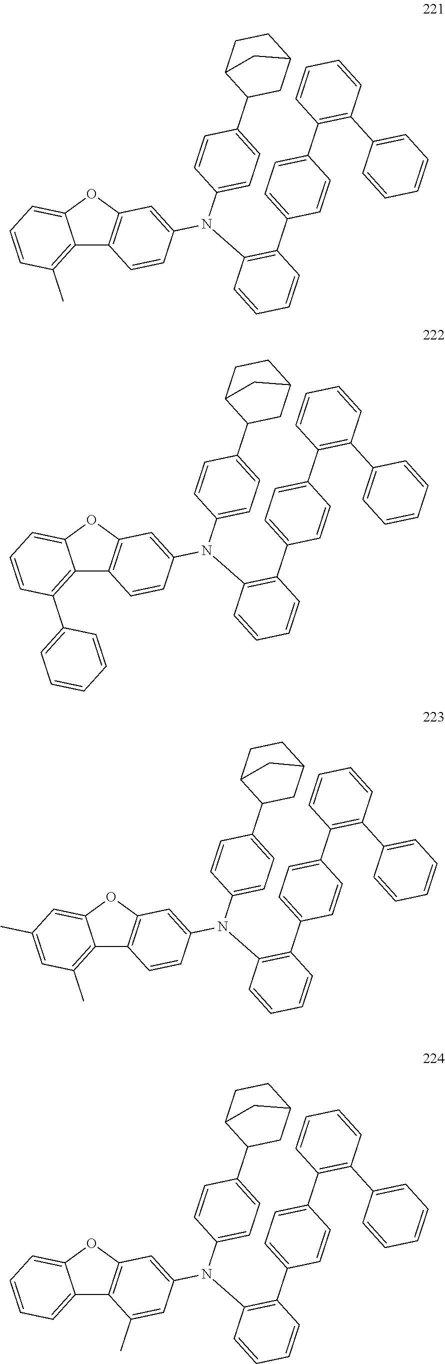 [SMILES: Cc1cccc2oc3cc(N(c4ccc(C5CC6CCC5C6)cc4)c4ccccc4-c4ccc(-c5ccccc5-c5ccccc5)cc4)ccc3c12 ; c1ccc(-c2ccccc2-c2ccc(-c3ccccc3N(c3ccc(C4CC5CCC4C5)cc3)c3ccc4c(c3)oc3cccc(-c5ccccc5)c34)cc2)cc1 ; Cc1cc(C)c2c(c1)oc1cc(N(c3ccc(C4CC5CCC4C5)cc3)c3ccccc3-c3ccc(-c4ccccc4-c4ccccc4)cc3)ccc12 ; Cc1cc(N(c2ccc(C3CC4CCC3C4)cc2)c2ccccc2-c2ccc(-c3ccccc3-c3ccccc3)cc2)cc2oc3ccccc3c12]

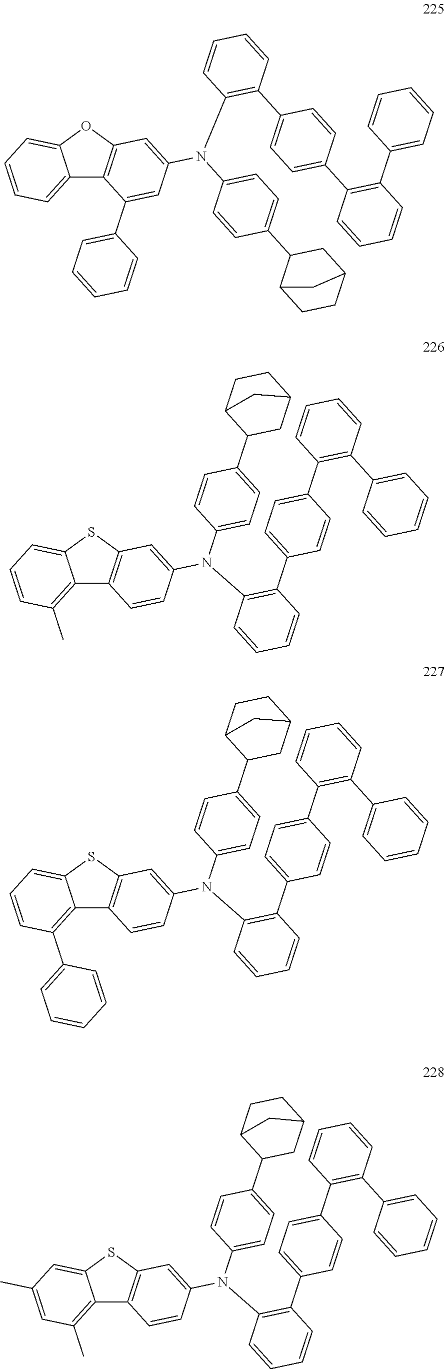

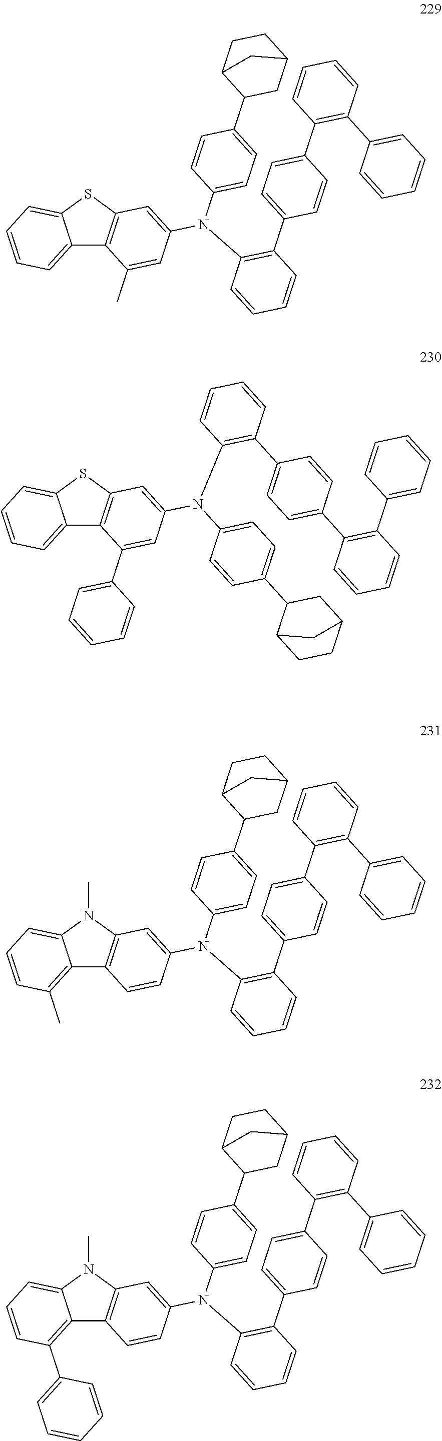 [SMILES: Cc1cc(N(c2ccc(C3CC4CCC3C4)cc2)c2ccccc2-c2ccc(-c3ccccc3-c3ccccc3)cc2)cc2sc3ccccc3c12 ; c1ccc(-c2ccccc2-c2ccc(-c3ccccc3N(c3ccc(C4CC5CCC4C5)cc3)c3cc(-c4ccccc4)c4c(c3)sc3ccccc34)cc2)cc1 ; Cc1cccc2c1c1ccc(N(c3ccc(C4CC5CCC4C5)cc3)c3ccccc3-c3ccc(-c4ccccc4-c4ccccc4)cc3)cc1n2C ; Cn1c2cc(N(c3ccc(C4CC5CCC4C5)cc3)c3ccccc3-c3ccc(-c4ccccc4-c4ccccc4)cc3)ccc2c2c(-c3ccccc3)cccc21]

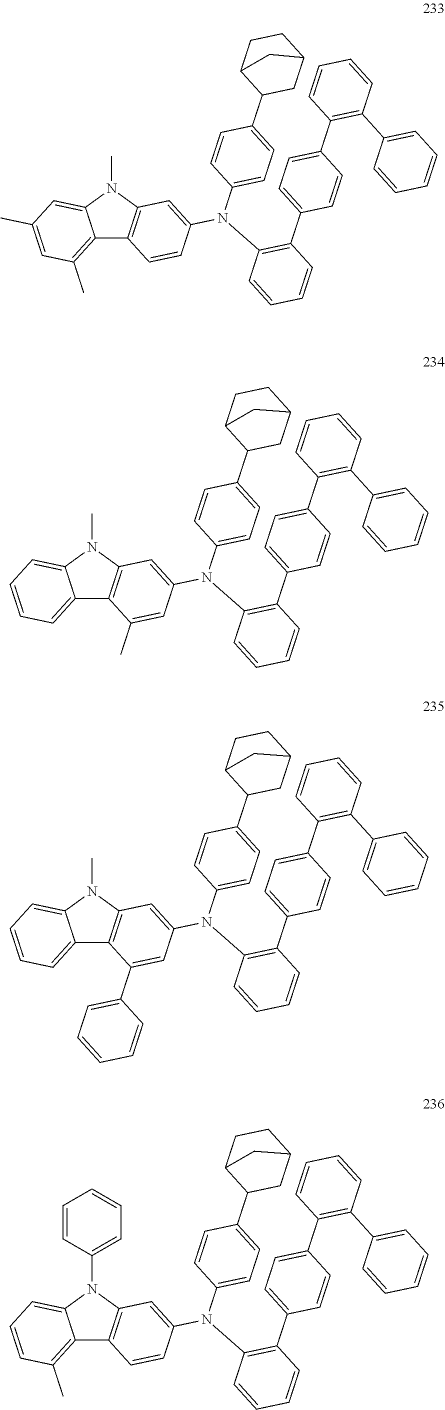 [SMILES: Cc1cc(C)c2c3ccc(N(c4ccc(C5CC6CCC5C6)cc4)c4ccccc4-c4ccc(-c5ccccc5-c5ccccc5)cc4)cc3n(C)c2c1 ; Cc1cc(N(c2ccc(C3CC4CCC3C4)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C4CC5CCC4C5)cc3)c3ccccc3-c3ccc(-c4ccccc4-c4ccccc4)cc3)cc21 ; Cc1cccc2c1c1ccc(N(c3ccc(C4CC5CCC4C5)cc3)c3ccccc3-c3ccc(-c4ccccc4-c4ccccc4)cc3)cc1n2-c1ccccc1]

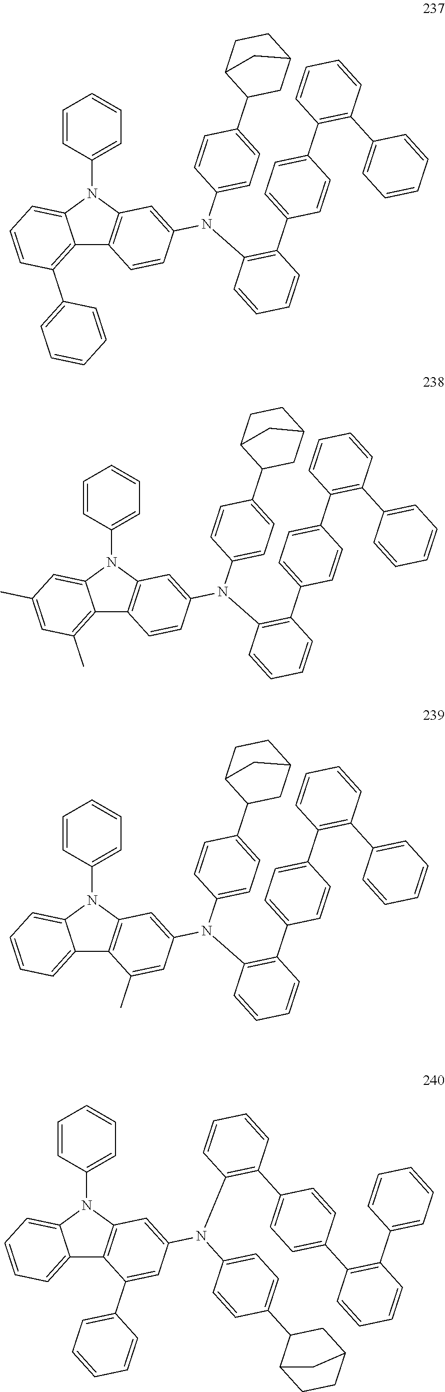 [SMILES: c1ccc(-c2ccccc2-c2ccc(-c3ccccc3N(c3ccc(C4CC5CCC4C5)cc3)c3ccc4c5c(-c6ccccc6)cccc5n(-c5ccccc5)c4c3)cc2)cc1 ; Cc1cc(C)c2c3ccc(N(c4ccc(C5CC6CCC5C6)cc4)c4ccccc4-c4ccc(-c5ccccc5-c5ccccc5)cc4)cc3n(-c3ccccc3)c2c1 ; Cc1cc(N(c2ccc(C3CC4CCC3C4)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CC5CCC4C5)cc3)c3cc(-c4ccccc4)c4c5ccccc5n(-c5ccccc5)c4c3)cc2)cc1]

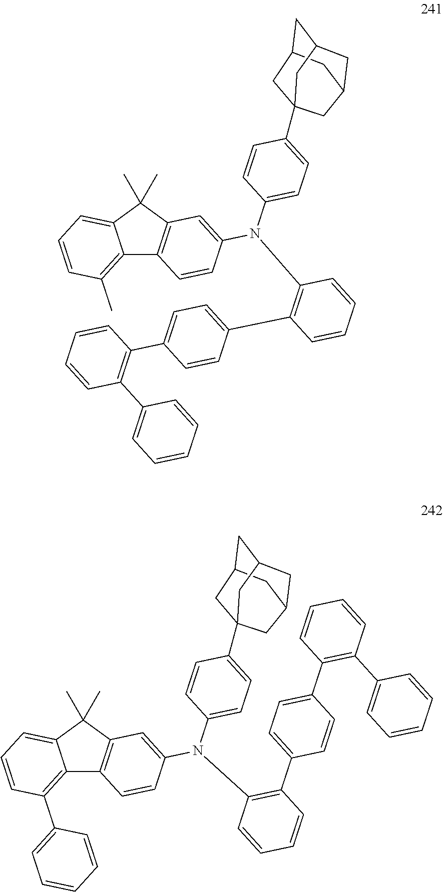 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(C)C ; CC1(C)c2cc(N(c3ccc(C45CC6CC(CC(C6)C4)C5)cc3)c3ccccc3-c3ccc(-c4ccccc4-c4ccccc4)cc3)ccc2-c2c(-c3ccccc3)cccc21]

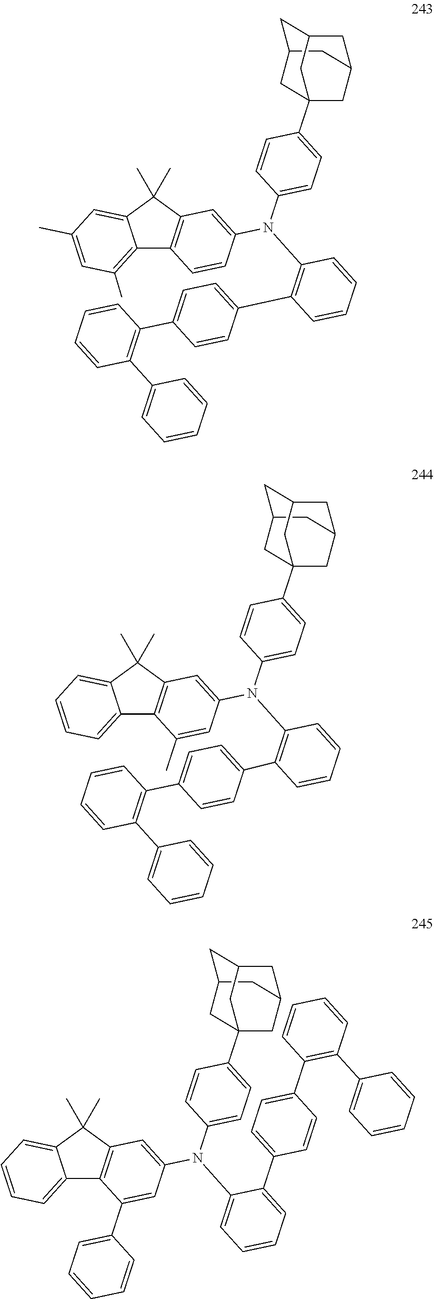 [SMILES: Cc1cc(C)c2c(c1)C(C)(C)c1cc(N(c3ccc(C45CC6CC(CC(C6)C4)C5)cc3)c3ccccc3-c3ccc(-c4ccccc4-c4ccccc4)cc3)ccc1-2 ; Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(C)C ; CC1(C)c2ccccc2-c2c(-c3ccccc3)cc(N(c3ccc(C45CC6CC(CC(C6)C4)C5)cc3)c3ccccc3-c3ccc(-c4ccccc4-c4ccccc4)cc3)cc21]

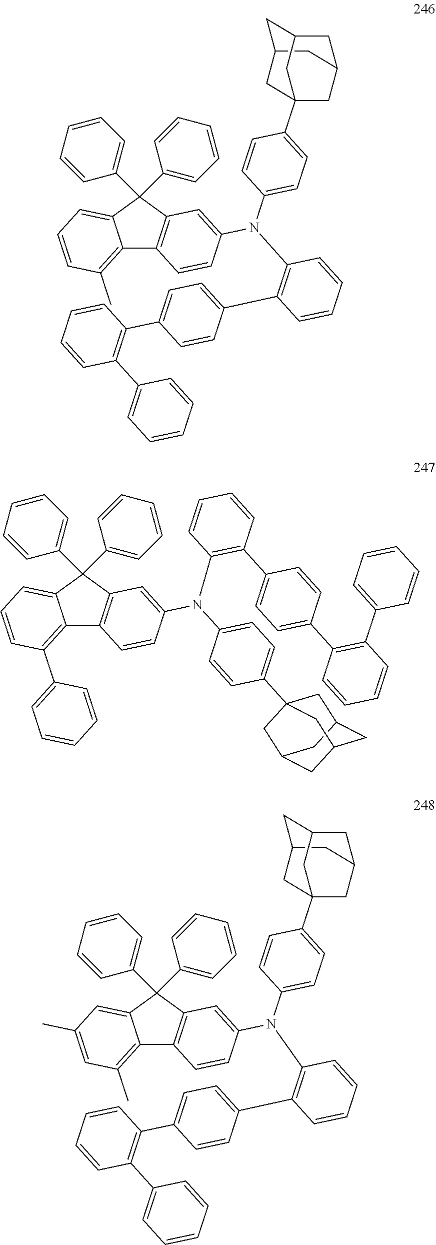 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C45CC6CC(CC(C6)C4)C5)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C45CC6CC(CC(C6)C4)C5)cc3)c3ccccc3-c3ccc(-c4ccccc4-c4ccccc4)cc3)ccc1-2]

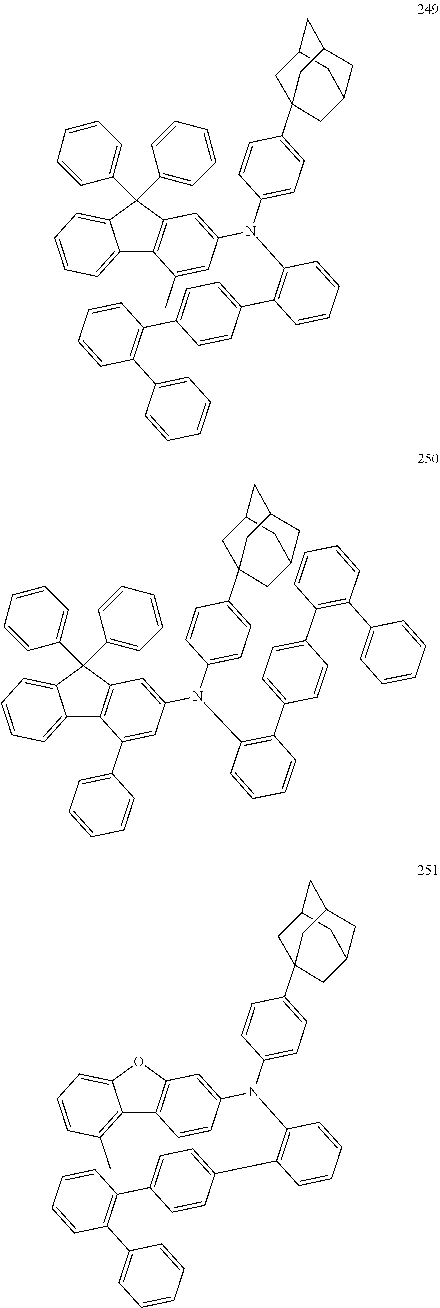 [SMILES: Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C45CC6CC(CC(C6)C4)C5)cc3)c3cc(-c4ccccc4)c4c(c3)C(c3ccccc3)(c3ccccc3)c3ccccc3-4)cc2)cc1 ; Cc1cccc2oc3cc(N(c4ccc(C56CC7CC(CC(C7)C5)C6)cc4)c4ccccc4-c4ccc(-c5ccccc5-c5ccccc5)cc4)ccc3c12]

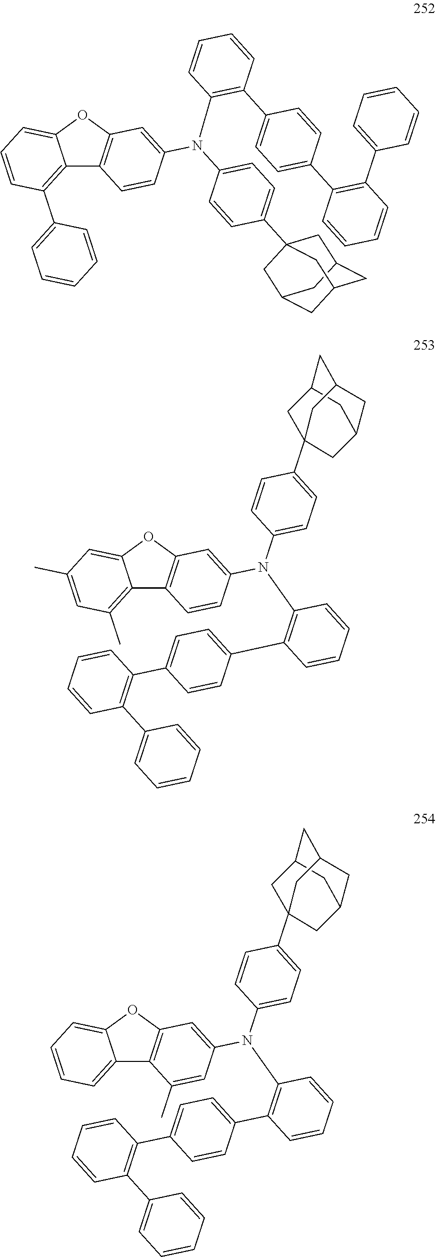

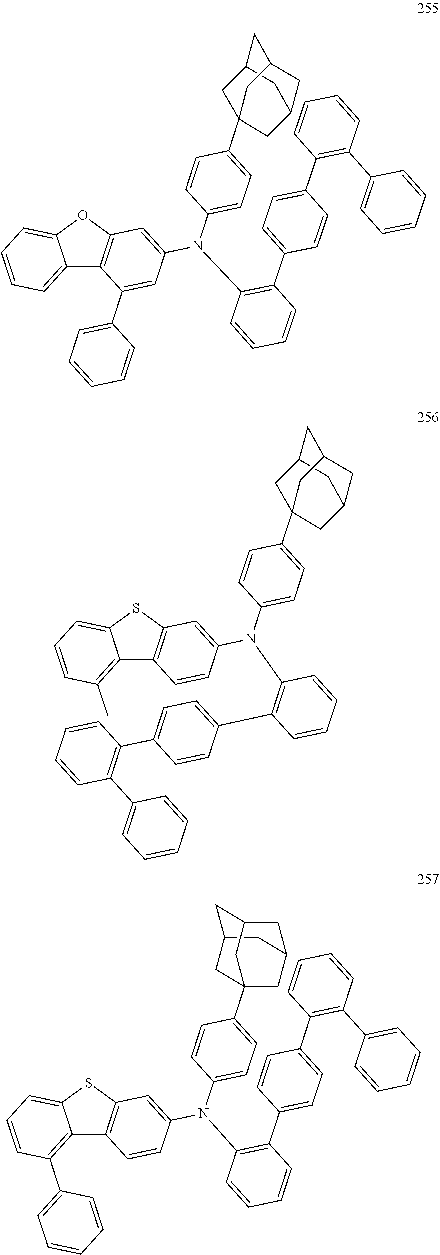 [SMILES: c1ccc(-c2ccccc2-c2ccc(-c3ccccc3N(c3ccc(C45CC6CC(CC(C6)C4)C5)cc3)c3cc(-c4ccccc4)c4c(c3)oc3ccccc34)cc2)cc1 ; Cc1cccc2sc3cc(N(c4ccc(C56CC7CC(CC(C7)C5)C6)cc4)c4ccccc4-c4ccc(-c5ccccc5-c5ccccc5)cc4)ccc3c12 ; c1ccc(-c2ccccc2-c2ccc(-c3ccccc3N(c3ccc(C45CC6CC(CC(C6)C4)C5)cc3)c3ccc4c(c3)sc3cccc(-c5ccccc5)c34)cc2)cc1]

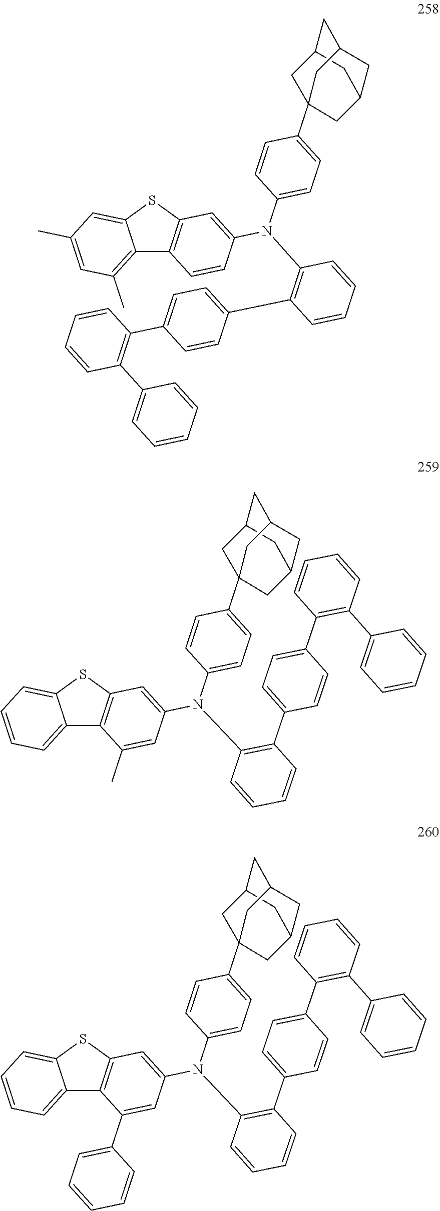 [SMILES: Cc1cc(C)c2c(c1)sc1cc(N(c3ccc(C45CC6CC(CC(C6)C4)C5)cc3)c3ccccc3-c3ccc(-c4ccccc4-c4ccccc4)cc3)ccc12 ; Cc1cc(N(c2ccc(C34CC5CC(CC(C5)C3)C4)cc2)c2ccccc2-c2ccc(-c3ccccc3-c3ccccc3)cc2)cc2sc3ccccc3c12 ; c1ccc(-c2ccccc2-c2ccc(-c3ccccc3N(c3ccc(C45CC6CC(CC(C6)C4)C5)cc3)c3cc(-c4ccccc4)c4c(c3)sc3ccccc34)cc2)cc1]

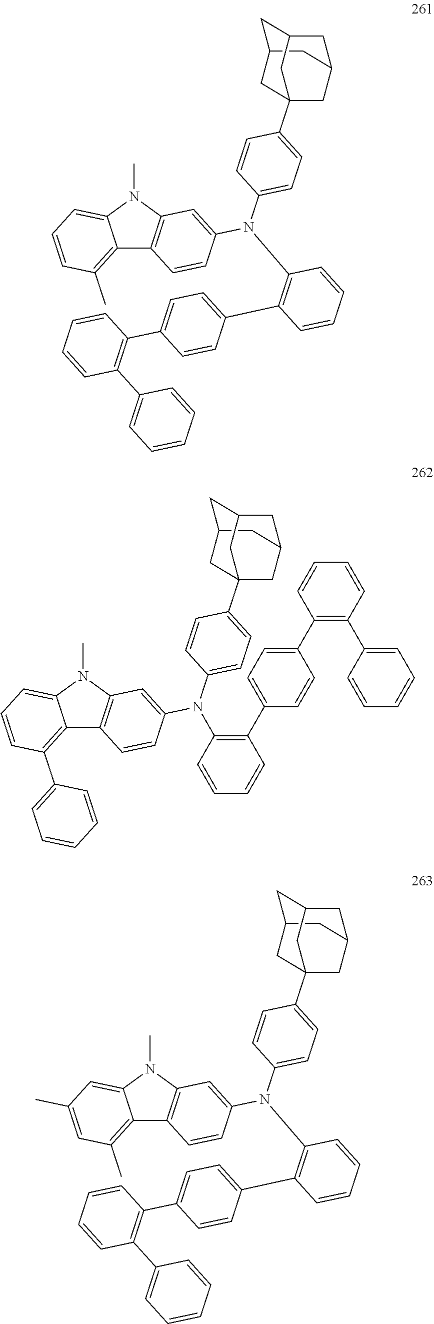 [SMILES: Cc1cccc2c1c1ccc(N(c3ccc(C45CC6CC(CC(C6)C4)C5)cc3)c3ccccc3-c3ccc(-c4ccccc4-c4ccccc4)cc3)cc1n2C ; Cn1c2cc(N(c3ccc(C45CC6CC(CC(C6)C4)C5)cc3)c3ccccc3-c3ccc(-c4ccccc4-c4ccccc4)cc3)ccc2c2c(-c3ccccc3)cccc21 ; Cc1cc(C)c2c3ccc(N(c4ccc(C56CC7CC(CC(C7)C5)C6)cc4)c4ccccc4-c4ccc(-c5ccccc5-c5ccccc5)cc4)cc3n(C)c2c1]

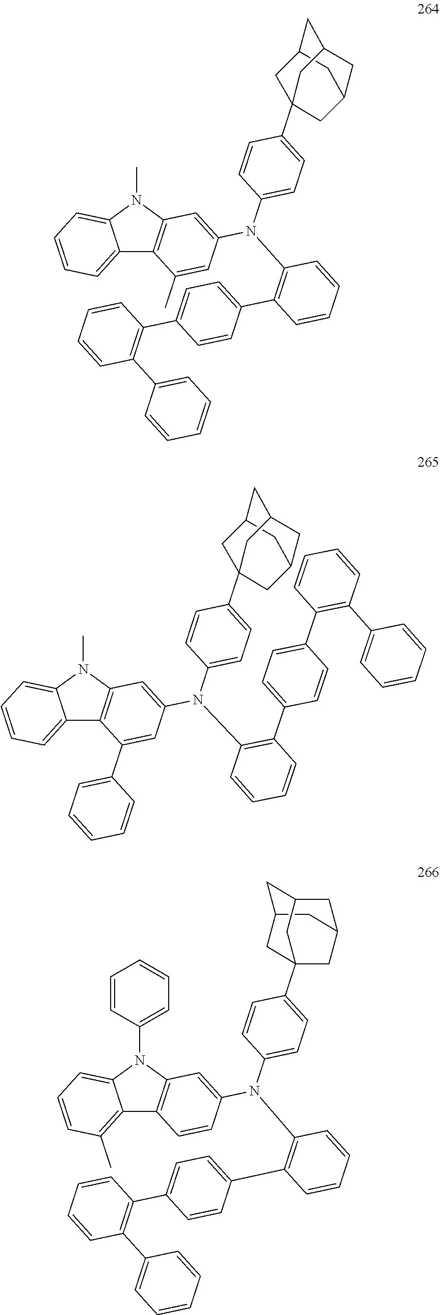 [SMILES: Cc1cc(N(c2ccc(C34CC5CC(CC(C5)C3)C4)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C45CC6CC(CC(C6)C4)C5)cc3)c3ccccc3-c3ccc(-c4ccccc4-c4ccccc4)cc3)cc21 ; Cc1cccc2c1c1ccc(N(c3ccc(C45CC6CC(CC(C6)C4)C5)cc3)c3ccccc3-c3ccc(-c4ccccc4-c4ccccc4)cc3)cc1n2-c1ccccc1]

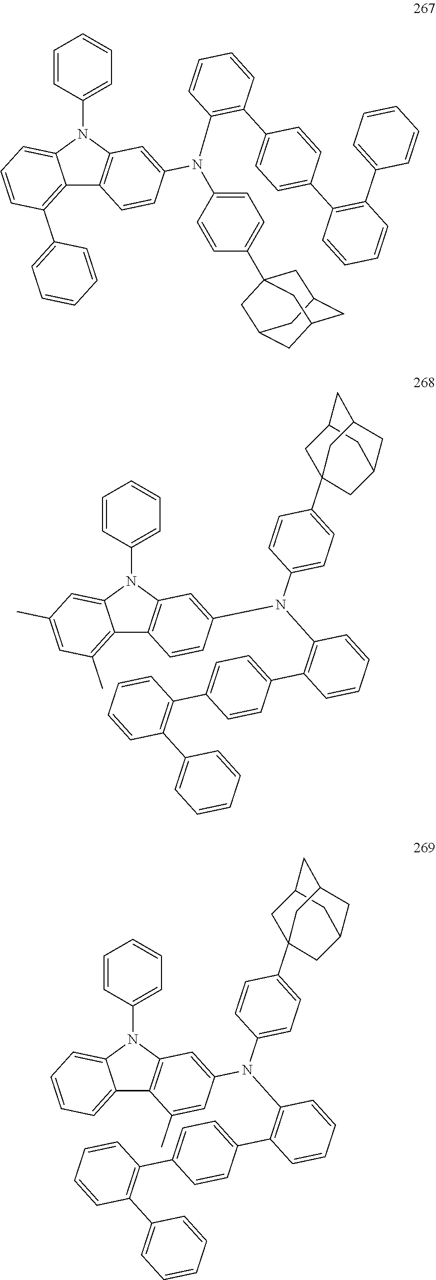 [SMILES: c1ccc(-c2ccccc2-c2ccc(-c3ccccc3N(c3ccc(C45CC6CC(CC(C6)C4)C5)cc3)c3ccc4c5c(-c6ccccc6)cccc5n(-c5ccccc5)c4c3)cc2)cc1 ; Cc1cc(C)c2c3ccc(N(c4ccc(C56CC7CC(CC(C7)C5)C6)cc4)c4ccccc4-c4ccc(-c5ccccc5-c5ccccc5)cc4)cc3n(-c3ccccc3)c2c1 ; Cc1cc(N(c2ccc(C34CC5CC(CC(C5)C3)C4)cc2)c2ccccc2-c2ccc(-c3ccccc3-c3ccccc3)cc2)cc2c1c1ccccc1n2-c1ccccc1]

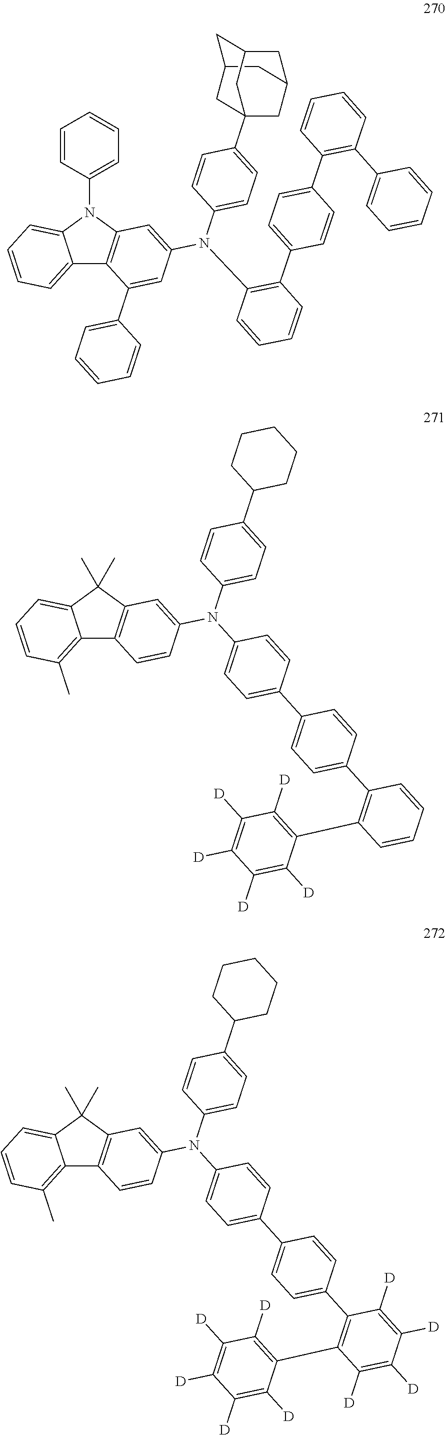 [SMILES: c1ccc(-c2ccccc2-c2ccc(-c3ccccc3N(c3ccc(C45CC6CC(CC(C6)C4)C5)cc3)c3cc(-c4ccccc4)c4c5ccccc5n(-c5ccccc5)c4c3)cc2)cc1 ; [2H]c1c([2H])c([2H])c(-c2ccccc2-c2ccc(-c3ccc(N(c4ccc(C5CCCCC5)cc4)c4ccc5c(c4)C(C)(C)c4cccc(C)c4-5)cc3)cc2)c([2H])c1[2H] ; [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H]]

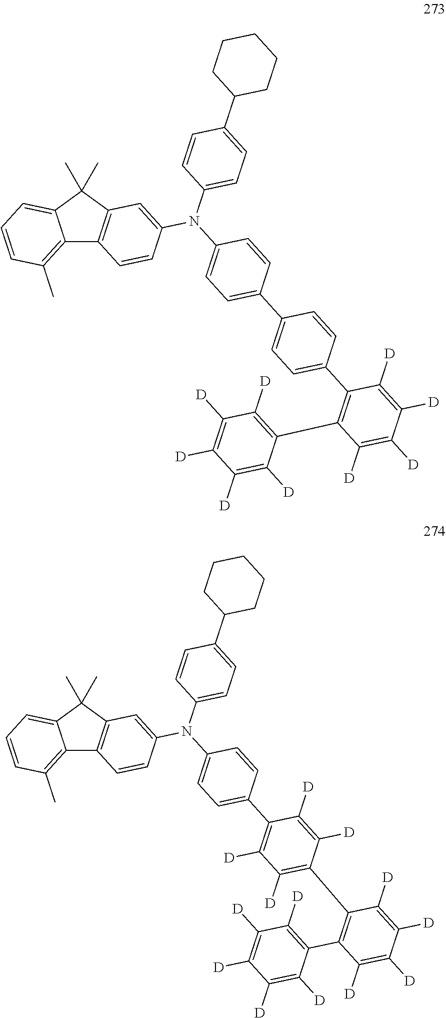 [SMILES: [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H] ; [2H]c1c([2H])c([2H])c(-c2c([2H])c([2H])c([2H])c([2H])c2-c2c([2H])c([2H])c(-c3ccc(N(c4ccc(C5CCCCC5)cc4)c4ccc5c(c4)C(C)(C)c4cccc(C)c4-5)cc3)c([2H])c2[2H])c([2H])c1[2H]]

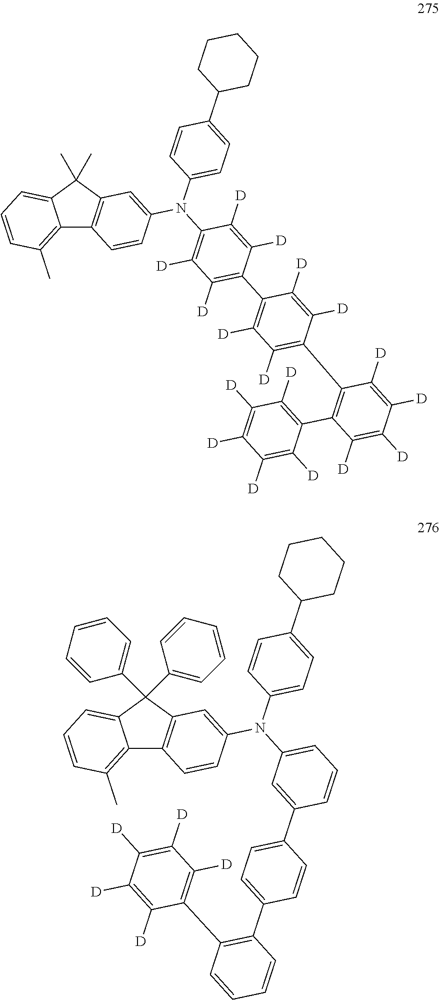 [SMILES: [2H]c1c([2H])c([2H])c(-c2c([2H])c([2H])c([2H])c([2H])c2-c2c([2H])c([2H])c(-c3c([2H])c([2H])c(N(c4ccc(C5CCCCC5)cc4)c4ccc5c(c4)C(C)(C)c4cccc(C)c4-5)c([2H])c3[2H])c([2H])c2[2H])c([2H])c1[2H] ; [2H]c1c([2H])c([2H])c(-c2ccccc2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H]]

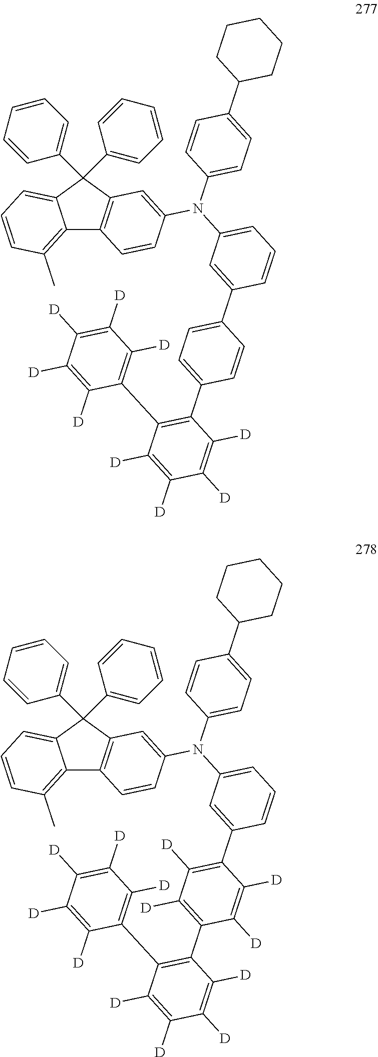 [SMILES: [2H]c1c([2H])c([2H])c(-c2c([2H])c([2H])c([2H])c([2H])c2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H] ; [2H]c1c([2H])c([2H])c(-c2c([2H])c([2H])c([2H])c([2H])c2-c2c([2H])c([2H])c(-c3cccc(N(c4ccc(C5CCCCC5)cc4)c4ccc5c(c4)C(c4ccccc4)(c4ccccc4)c4cccc(C)c4-5)c3)c([2H])c2[2H])c([2H])c1[2H]]

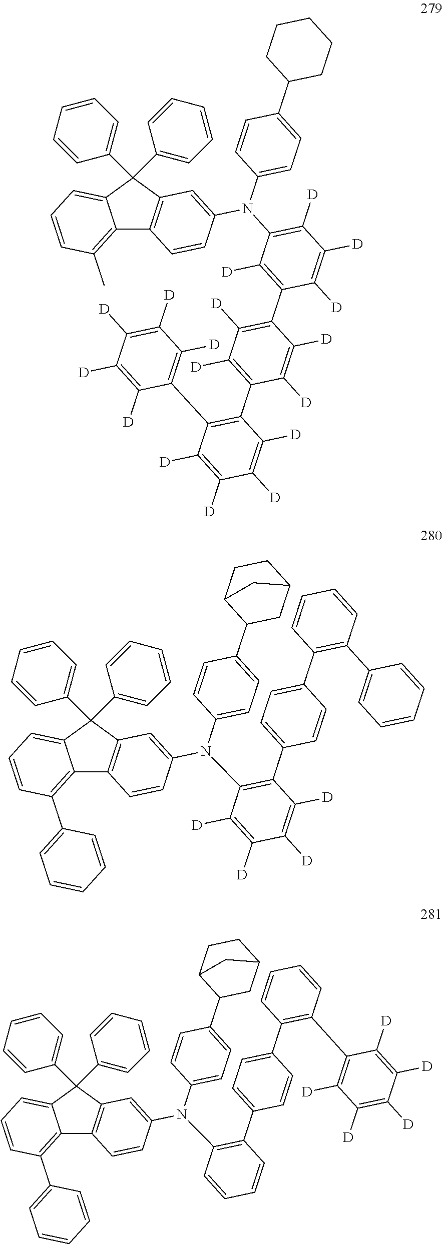 [SMILES: [2H]c1c([2H])c([2H])c(-c2c([2H])c([2H])c([2H])c([2H])c2-c2c([2H])c([2H])c(-c3c([2H])c([2H])c([2H])c(N(c4ccc(C5CCCCC5)cc4)c4ccc5c(c4)C(c4ccccc4)(c4ccccc4)c4cccc(C)c4-5)c3[2H])c([2H])c2[2H])c([2H])c1[2H] ; [2H]c1c([2H])c([2H])c(N(c2ccc(C3CC4CCC3C4)cc2)c2ccc3c(c2)C(c2ccccc2)(c2ccccc2)c2cccc(-c4ccccc4)c2-3)c(-c2ccc(-c3ccccc3-c3ccccc3)cc2)c1[2H] ; [2H]c1c([2H])c([2H])c(-c2ccccc2-c2ccc(-c3ccccc3N(c3ccc(C4CC5CCC4C5)cc3)c3ccc4c(c3)C(c3ccccc3)(c3ccccc3)c3cccc(-c5ccccc5)c3-4)cc2)c([2H])c1[2H]]

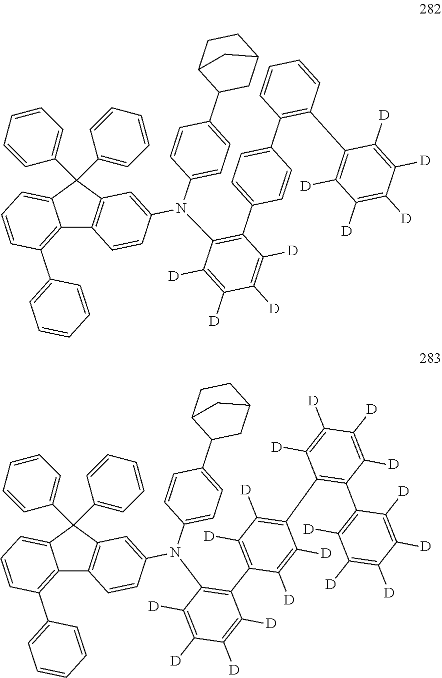 [SMILES: [2H]c1c([2H])c([2H])c(-c2ccccc2-c2ccc(-c3c([2H])c([2H])c([2H])c([2H])c3N(c3ccc(C4CC5CCC4C5)cc3)c3ccc4c(c3)C(c3ccccc3)(c3ccccc3)c3cccc(-c5ccccc5)c3-4)cc2)c([2H])c1[2H] ; [2H]c1c([2H])c([2H])c(-c2c([2H])c([2H])c([2H])c([2H])c2-c2c([2H])c([2H])c(-c3c([2H])c([2H])c([2H])c([2H])c3N(c3ccc(C4CC5CCC4C5)cc3)c3ccc4c(c3)C(c3ccccc3)(c3ccccc3)c3cccc(-c5ccccc5)c3-4)c([2H])c2[2H])c([2H])c1[2H]]

The amine compound according to an embodiment has a structure in which a first substituent, a second substituent, and a third substituent are connected to a core nitrogen atom, and the first substituent contains a dibenzofuranyl moiety, a dibenzothiophenyl moiety, a carbazole moiety, or a fluorenyl moiety. The second substituent has a structure in which a cyclohexyl group, a bicycloheptanyl group, or an aliphatic cycloalkyl group of an adamantyl group is connected via an arylene linker. The third substituent has a substituted or unsubstituted quarterphenyl group structure. In the third substituent, a benzene ring of the quarterphenyl group bonded to a nitrogen atom is named as a first benzene ring, and benzene rings are sequentially named as second, third, and fourth benzene rings from the benzene ring connected to the first benzene ring. In this case, the third benzene ring is bonded at a para position with respect to the carbon atom among ring-forming carbon atoms of the second benzene ring, which forms a bond between the first benzene ring and the second benzene ring. In some embodiments, the fourth benzene ring is bonded at an ortho position with respect to the carbon atom among ring-forming carbon atoms of the third benzene ring, which forms a bond with the second benzene ring. The amine compound according to an embodiment has the first to third substituents, thereby improving the hole-transporting properties and stability of a molecule when applied to the hole transport layer. Therefore, when the amine compound according to an embodiment is applied to the light-emitting element ED or the display device DD, improvement in luminous efficiency and long-lifespan may be achieved.

When the amine compound according to an embodiment is utilized in the hole transport region HTR, an energy level of HOMO may be variously changed by varying the substituent between the first electrode EL1 and the second electrode EL2. Accordingly, when the amine compound according to an embodiment is utilized in the hole transport region HTR, a hole injection barrier is variously changed, thereby making it possible to increase exciton generation efficiency inside the light-emitting layer EML. In some embodiments, a refractive index of the hole transport region HTR is adjusted by varying a substituent that may be substituted for a nitrogen atom included in the amine compound according to an embodiment, thereby making it possible to improve external quantum efficiency. As described above, when the amine compound according to an embodiment is utilized in the hole transport region HTR, the luminous efficiency of the light-emitting element ED may increase, and the lifespan of the light-emitting element ED may be improved.

The hole transport region HTR may further include a compound represented by Formula H-1.

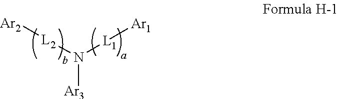

In Formula H-1, L₁ and L₂ may each independently be a direct linkage, a substituted or unsubstituted arylene group having 6 to 30 ring-forming carbon atoms or a substituted or unsubstituted heteroarylene group having 2 to 30 ring-forming carbon atoms. a and b may each independently be an integer of 0 to 10. In some embodiments, when a or b is an integer of 2 or more, a plurality of L₁s and L₂s may each independently be a substituted or unsubstituted arylene group having 6 to 30 ring-forming carbon atoms or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

The compound represented by Formula H-1 may be a monoamine compound. In some embodiments, the compound represented by Formula H-1 may be a diamine compound in which at least one selected from among Ar₁ to Ar₃ includes an amine group as a substituent. In some embodiments, the compound represented by Formula H-1 may be a carbazole-based compound containing a substituted or unsubstituted carbazole group in at least one selected from among Ar₁ and Ar₂ or a fluorene-based compound containing a substituted or unsubstituted fluorene group in at least one of Ar₁ or Ar₂.

The compound represented by Formula H-1 may be represented by any one selected from among compounds in Compound Group H. However, compounds presented in Compound Group H are for illustrative purposes, but the compound represented by Formula H-1 is not limited to the compounds presented in Compound Group H.

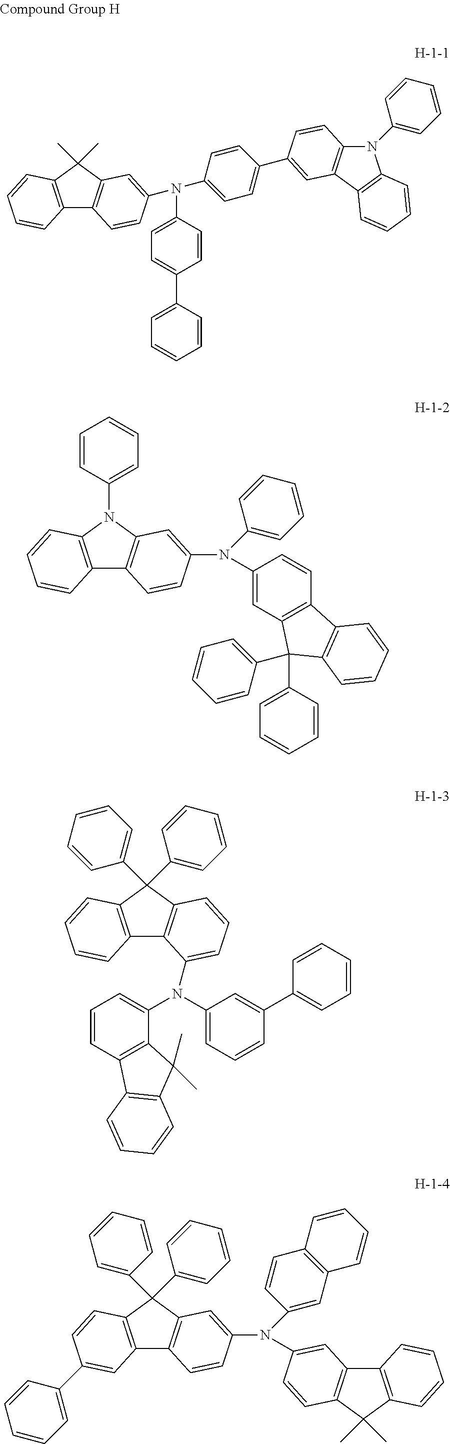 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

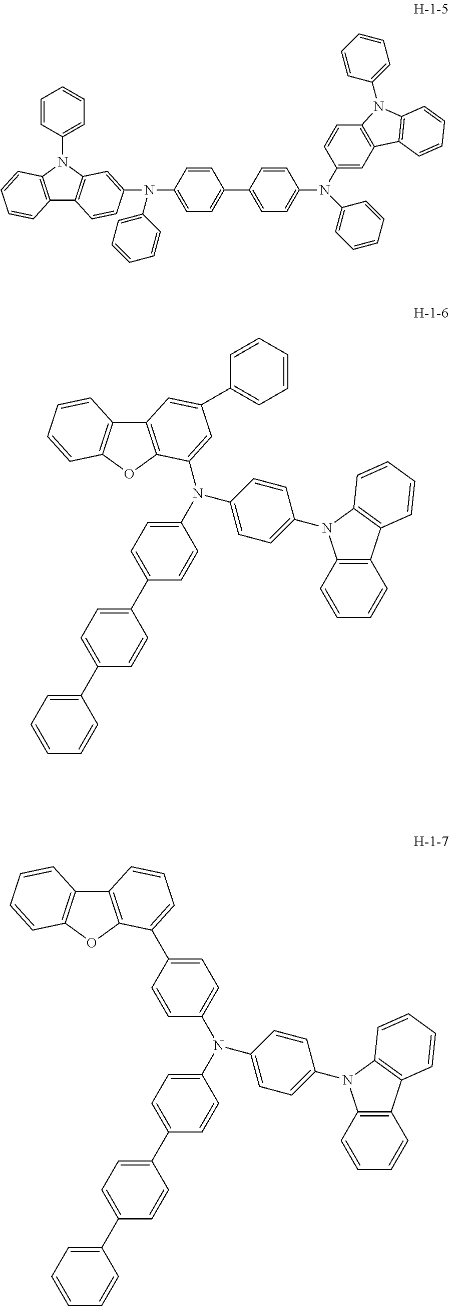 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

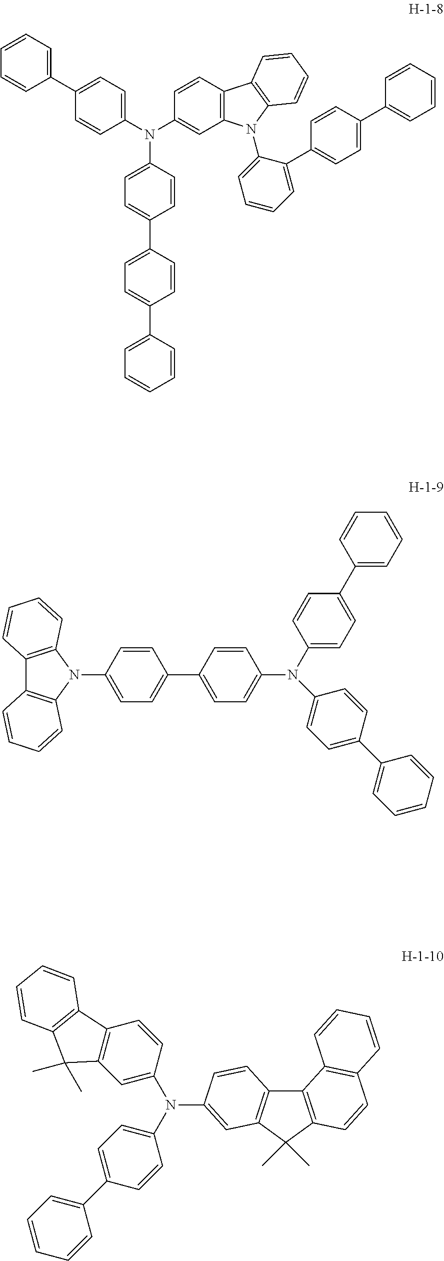 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

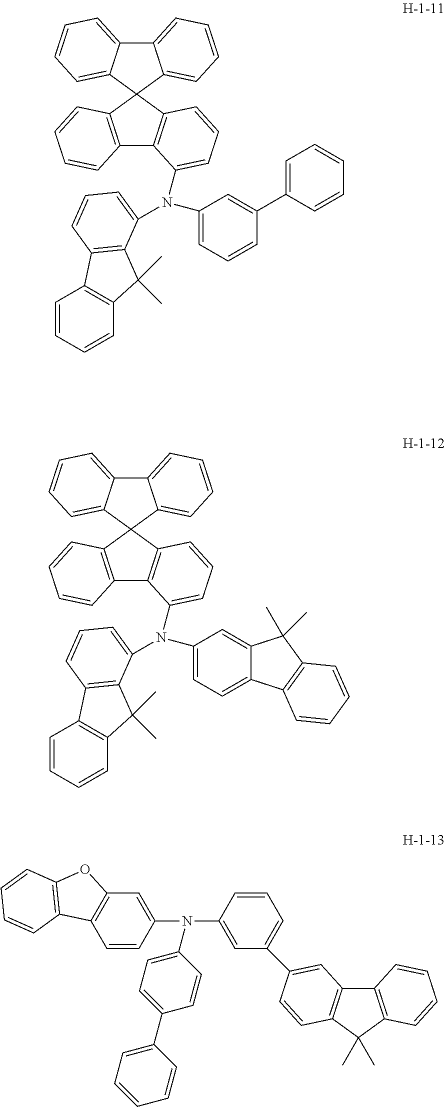 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

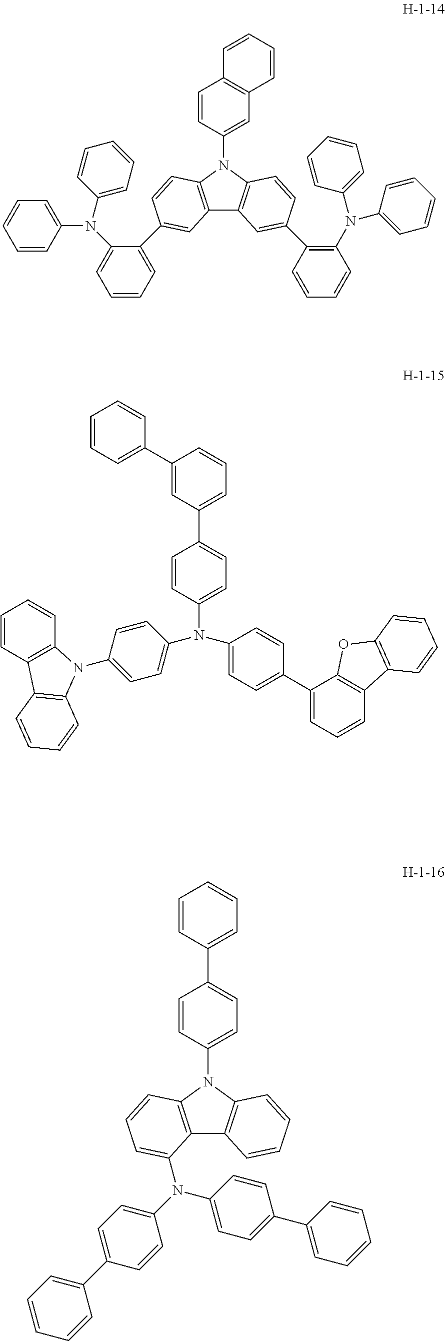 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

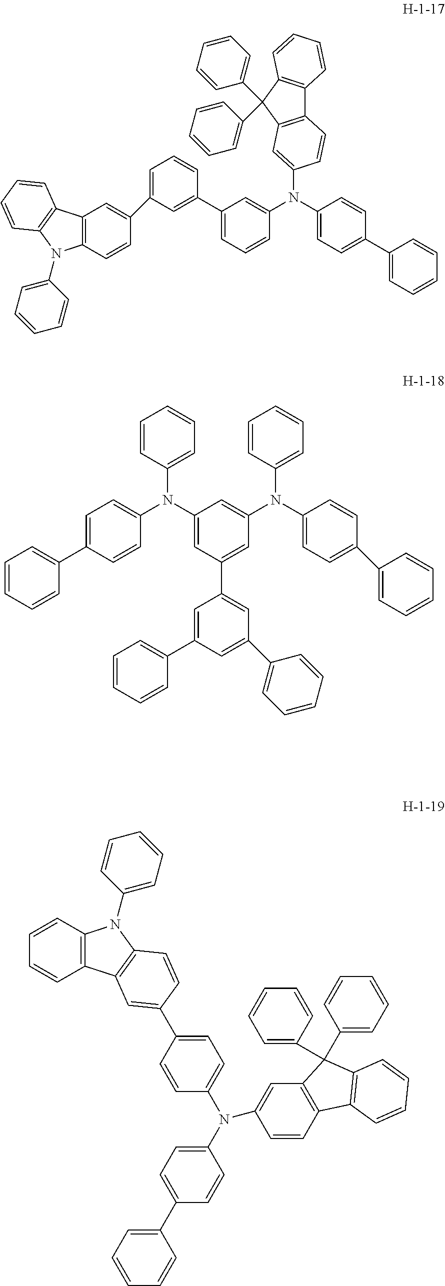 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-1-yl)-N,N′-diphenyl-benzidine (NPB), polyetherketone containing triphenylamine (TPAPEK), 4-isopropyl-4′-methyldiphenyliodonium [tetrakis(pentafluorophenyl)borate], dipyrazino[2,3-f: 2′,3′-h] quinoxaline-2,3,6,7,10,11-hexacarbonitrile (HATCN), etc.

The hole transport region HTR may also include: a carbazole-based derivative such as N-phenylcarbazole and polyvinylcarbazole; a fluorene-based derivative; a triphenylamine-based derivative such as N,N′-bis(3-methylphenyl)-N,N′-diphenyl-[1,1′-biphenyl]-4,4′-diamine (TPD) and 4,4′,4″-tris(N-carbazolyl)triphenylamine (TCTA); N,N′-di(naphthalene-1-yl)-N,N′-diphenyl-benzidine (NPB); 4,4′-cyclohexylidene bis[N,N-bis(4-methylphenyl)benzenamine] (TAPC); 4,4′-bis[N,N′-(3-tolyl)amino]-3,3′-dimethylbiphenyl (HMTPD); 1,3-bis(N-carbazolyl)benzene) (mCP); etc.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one selected from among the hole injection layer HIL, the hole transport layer HTL, and the electron-blocking layer EBL.

The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes a hole transport layer HTL, the hole transport layer HTL may have a thickness of about 30 Å to about 1000 Å. For example, when the hole transport region HTR includes an electron-blocking layer EBL, the electron-blocking layer EBL may have a thickness of about 10 Å to about 1000 Å. When the thicknesses of the hole transport region HTR, the hole injection layer HIL, the hole transport layer HTL, and the electron-blocking layer EBL fall within the above-described ranges, hole transport properties in a satisfying level may be obtained without a substantial increase in a driving voltage.

The hole transport region HTR, in addition to the above-described materials, may further include a charge generation material for improving conductivity. The charge generation material may be uniformly or nonuniformly dispersed in the hole transport region HTR. The charge generation material may be, for example, a p-dopant. The p-dopant may include at least one selected from among a halide metal compound, a quinone derivative, a metal oxide, and a cyano group-containing compound but is not limited thereto. For example, examples of the p-dopant may contain a metal halide compound such as CuI and RbI, a quinone derivative such as tetracyanoquinodimethane (TCNQ) and 2,3,5,6-tetrafluoro-7,7′8,8-tetracyanoquinodimethane (F4-TCNQ), a metal oxide such as tungsten oxide and molybdenum oxide, dipyrazino[2,3-f: 2′,3′-h] quinoxaline-2,3,6,7,10,11-hexacarbonitrile (HATCN) and a cyano group-containing compound such as 4-[[2, 3-bis[cyano-(4-cyano-2,3,5,6-tetrafluorophenyl)methylidene]cyclopropylidene]-cyanomethyl]-2,3,5,6-tetrafluorobenzonitrile (NDP9), etc. However, an embodiment of the present disclosure is not limited thereto.

As described above, the hole transport region HTR may further include at least one selected from among a buffer layer (not illustrated), and an electron-blocking layer EBL in addition to the hole injection layer HIL and the hole transport layer HTL. The buffer layer may increase luminous efficiency by compensating for a resonance distance according to a wavelength of light emitted from the light-emitting layer EML. As a material included in the buffer layer, a material that may be included in the hole transport region HTR may be utilized. The electron-blocking layer EBL serves to prevent or reduce electrons from being injected into the hole transport region HTR from the electron transport region ETR.

The light-emitting layer EML is provided on the hole transport region HTR. The light-emitting layer EML may have a thickness of, for example, about 100 Å to about 1000 Å, or about 100 Å to about 300 Å. The light-emitting layer EML may have a single layer formed of a single material, a single layer formed of a plurality of different materials, or a multi-layer structure including multiple layers formed of a plurality of different materials.

In the light-emitting element ED illustrated in FIGS. 3 to 6, the light-emitting layer EML may include a dopant. The light-emitting layer EML may contain a compound represented by Formula M-a. The compound represented by Formula M-a may be utilized as a phosphorescent dopant material.

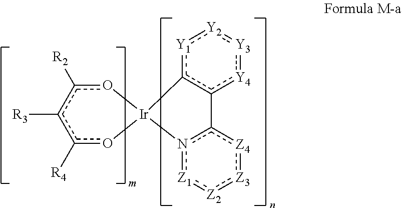

In Formula M-a, Y₁ to Y₄, and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or be bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as the phosphorescent dopant.

The compound represented by Formula M-a may be represented by any one selected from among Compounds M-a1 to M-a25. However, Compounds M-a1 to M-a25 are presented for illustrative purposes, but the compound represented by Formula M-a is not limited to compounds presented in Compounds M-a1 to M-a25.

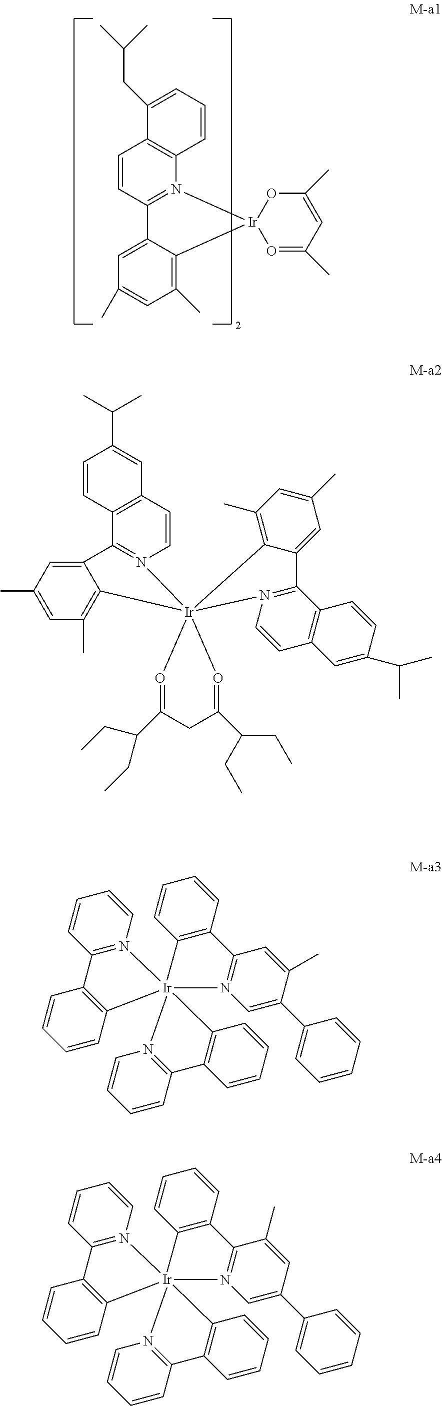

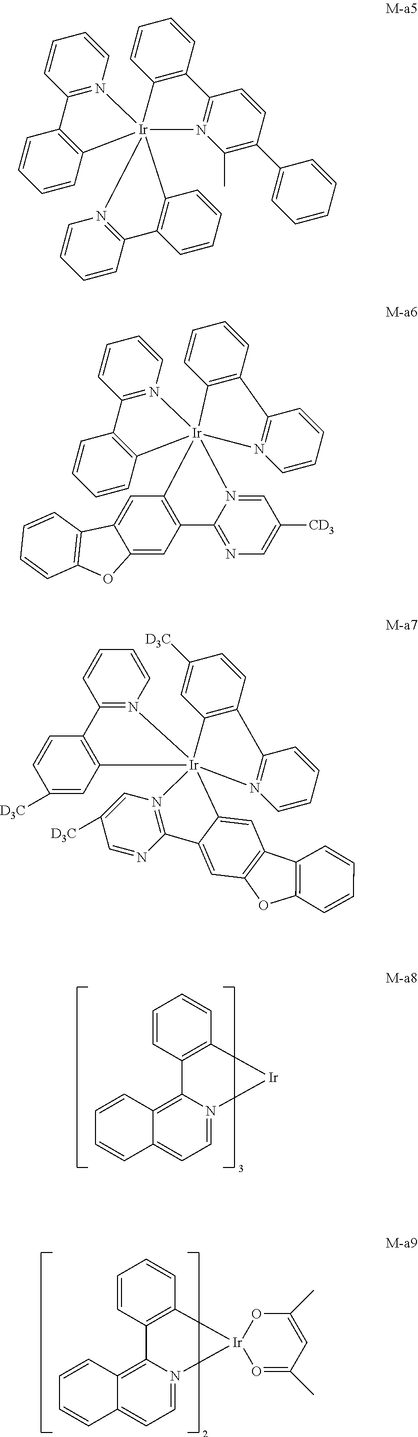

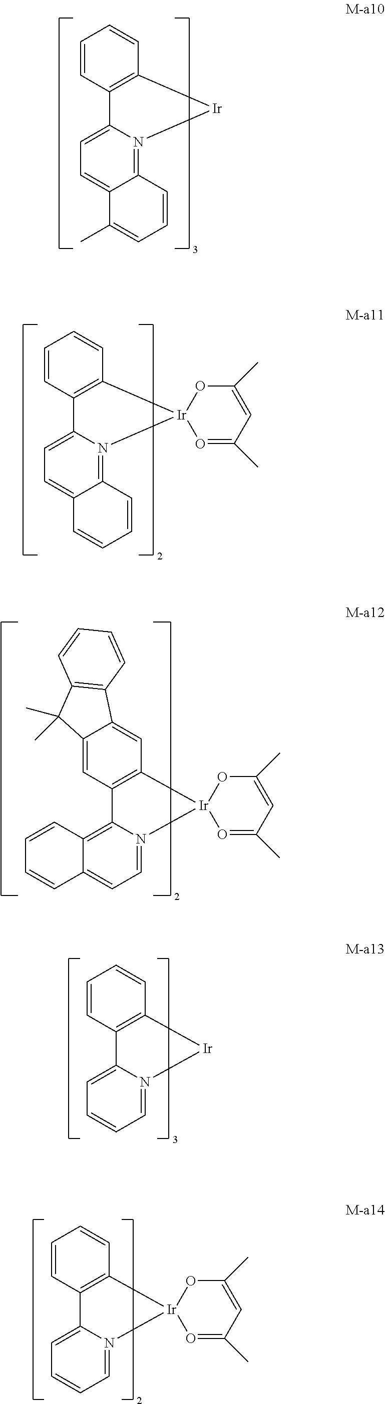

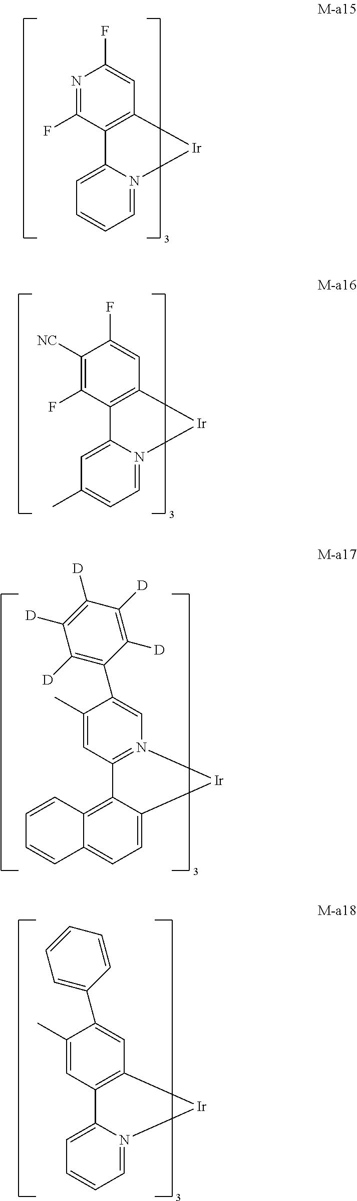

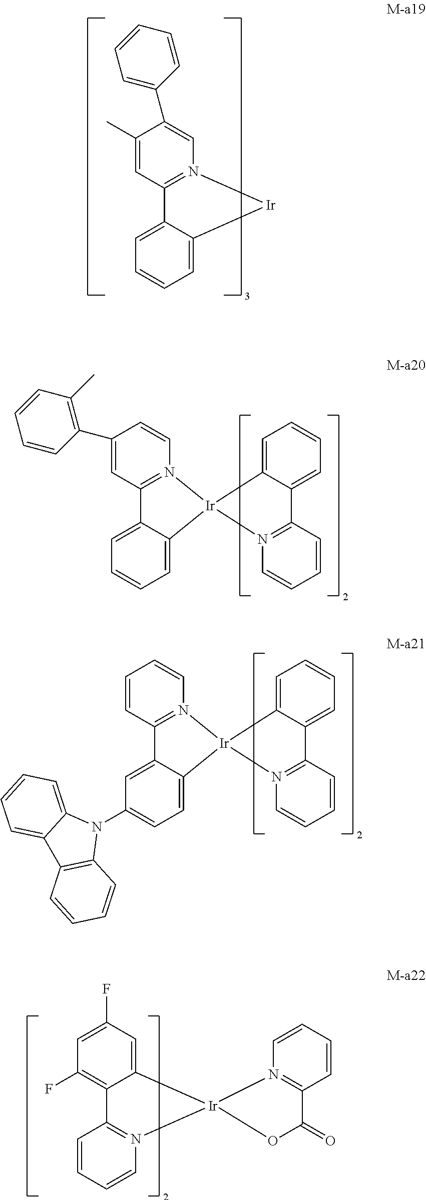

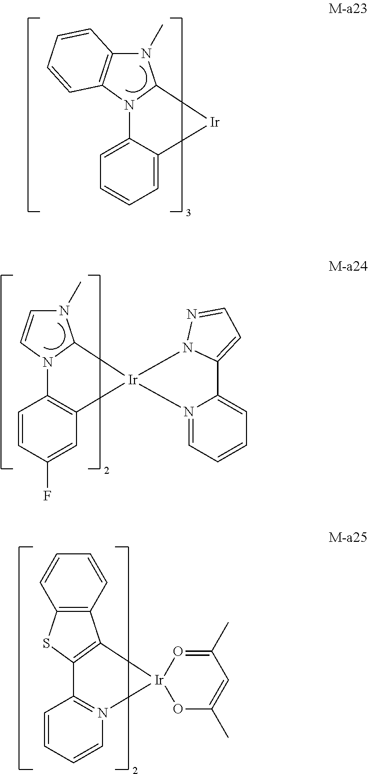

The light-emitting layer EML may include a first compound represented by any one selected from among Formulas F-a to F-c. The first compound represented by any one selected from among Formulas F-a to F-c may be utilized as the phosphorescent dopant material.

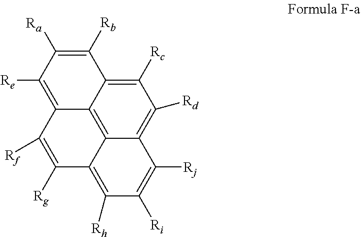

In Formula F-a, two selected from among Rₐ to Rᵢ may each independently be substituted with *—NAr₁Ar₂. The rest among Rₐ to Rᵢ, which is unsubstituted with *—NAr₁Ar₂, may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one selected from among Ar₁ and Ar₂ may be a heteroaryl group that contains 0 or S as a ring-forming atom.

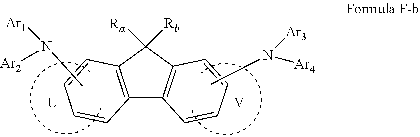

In Formula F-b, Rₐ and Rb may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted hetero ring having 2 to 30 ring-forming carbon atoms. At least one selected from among Ar₁ to Ar₄ may be a heteroaryl group that contains O or S as a ring-forming atom.

In Formula F-b, the number of rings represented by U and V may each independently be 0 or 1. For example, when the number of U or V is 1 in Formula F-b, one ring forms a fused ring at the designated part by U or V, and when the number of U or V is 0, no ring is present at the designated part by U or V. For example, when the number of U is 0 and the number of V is 1, or when the number of U is 1 and the number of V is 0, a fused ring having a fluorene core in Formula F-b may be a ring compound with four rings. In some embodiments, when the number of both (e.g., simultaneously) U and V is 0, the fused ring in Formula F-b may be a ring compound with three rings. In some embodiments, when the number of both (e.g., simultaneously) U and V is 1, the fused ring having the fluorene core in Formula F-b may be a ring compound with five rings.

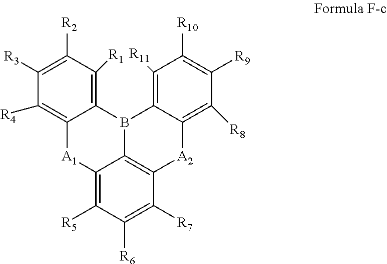

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or be bonded to an adjacent group to form a ring.

In Formula F-c, A₁ and A₂ may each independently be bonded to substituents of an adjacent group to form a fused ring. For example, when A₁ and A₂ may each independently be NRₘ, A₁ may be bonded to R₄ or R₅ to form a ring. In some embodiments, A₂ may be bonded to R₇ or R₈ to form a ring.

In an embodiment, the light-emitting layer EML, as suitable dopant materials, may include a styryl derivative (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi)), 4,4′-bis[2-(4-(N,N-diphenylamino)phenyl)vinyl]biphenyl (DPAVBi)), perylene and a derivative thereof (for example, 2, 5, 8, 11-tetra-t-butylperylene (TBP)), pyrene and a derivative thereof (for example, 1, 1-dipyrene, 1, 4-dipyrenylbenzene, 1, 4-bis(N, N-diphenylamino)pyrene), etc.

The light-emitting layer EML may include a suitable phosphorescent dopant material. For example, as the phosphorescent dopant, a metal complex containing iridium (Ir), platinum (Pt), osmium (Os), gold (Au), titanium (Ti), zirconium (Zr), hafnium (Hf), europium (Eu), terbium (Tb) or thulium (Tm) may be utilized. For example, bis(4,6-difluorophenylpyridinato-N,C2′)picolinate), bis(2,4-difluorophenylpyridinato)-tetrakis(1-pyrazolyl)borate iridium(III) (Fir6), or platinum octaethyl porphyrin (PtOEP) may be utilized as the phosphorescent dopant. However, an embodiment of the present disclosure is not limited thereto.

In the light-emitting element ED according to an embodiment, the light-emitting layer EML may include an anthracene derivative, a pyrene derivative, a fluoranthene derivative, a chrysene derivative, a dihydrobenzanthracene derivative, or a triphenylene derivative. For example, the light-emitting layer EML may include an anthracene derivative or a pyrene derivative.

In the light-emitting element ED illustrated in FIGS. 3 to 6, the light-emitting layer EML may include a host, and the light-emitting layer EML may include a compound represented by Formula E-1. The compound represented by Formula E-1 may be utilized as a phosphorescent host material.

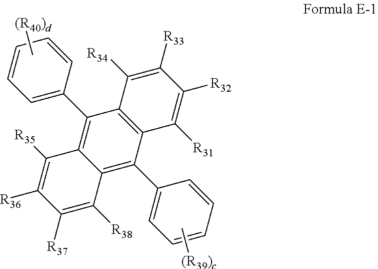

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or be bonded to an adjacent group to form a ring. In some embodiments, R₃₁ to R₄₀ may be bonded to an adjacent group to form a saturated hydrocarbon ring, an unsaturated hydrocarbon ring, a saturated hetero ring or an unsaturated hetero ring.

In Formula E-1, c and d may each independently be an integer of 0 to 5.

Formula E-1 may be represented by any one selected from among Compounds E1 to E19.

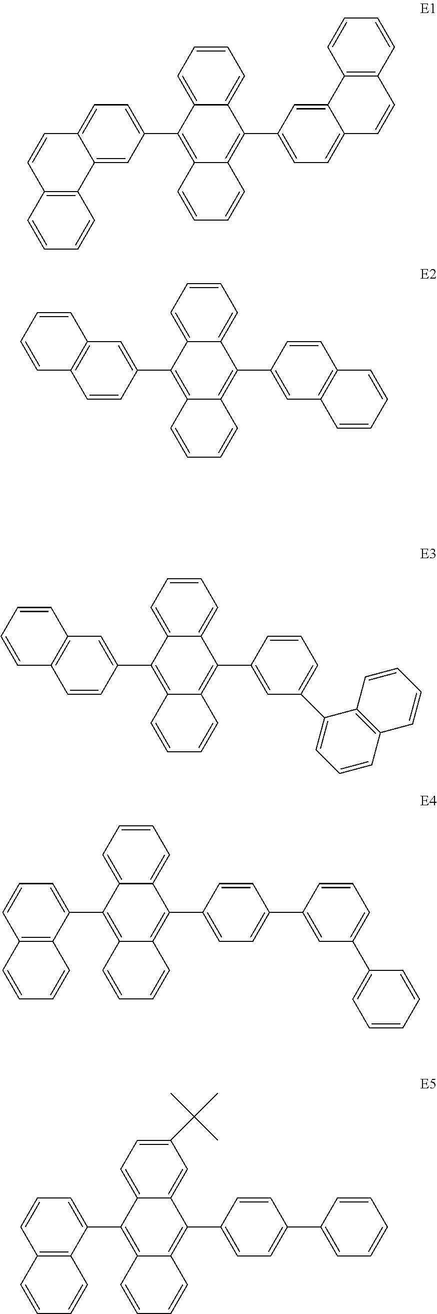

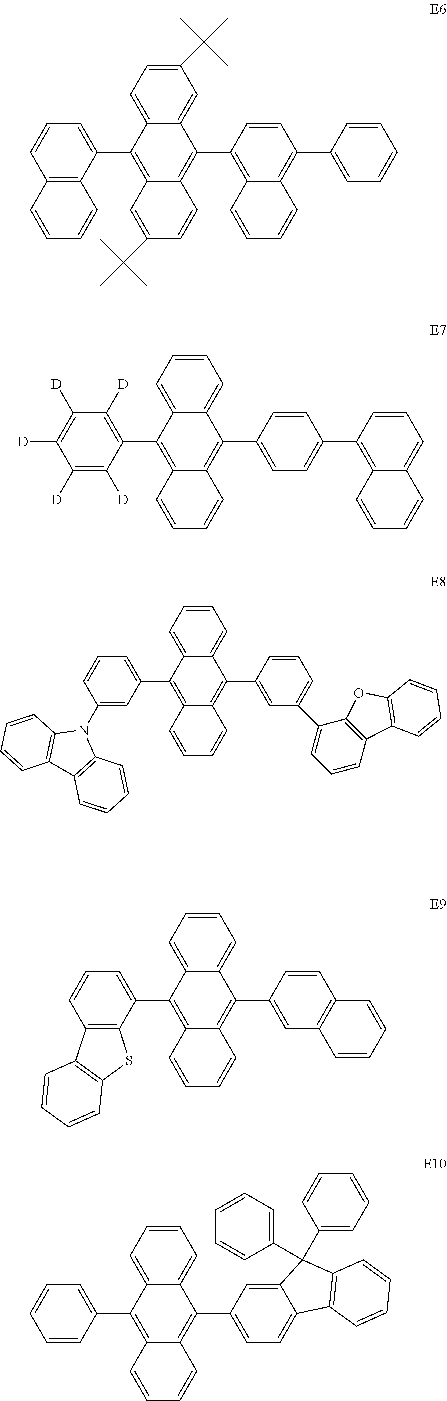

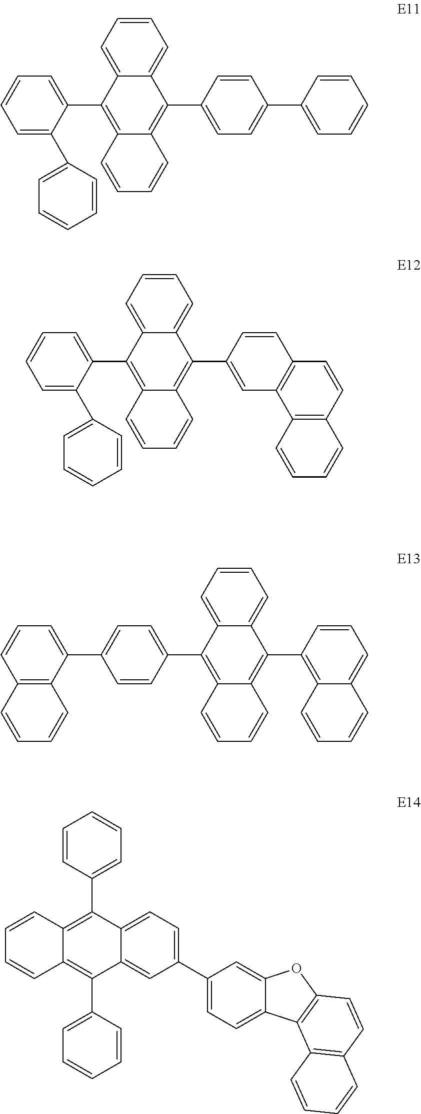

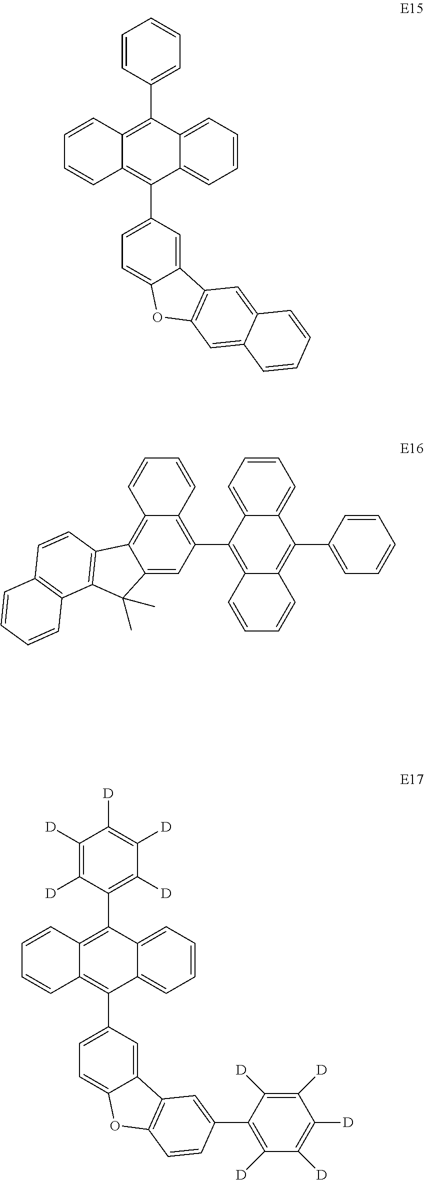

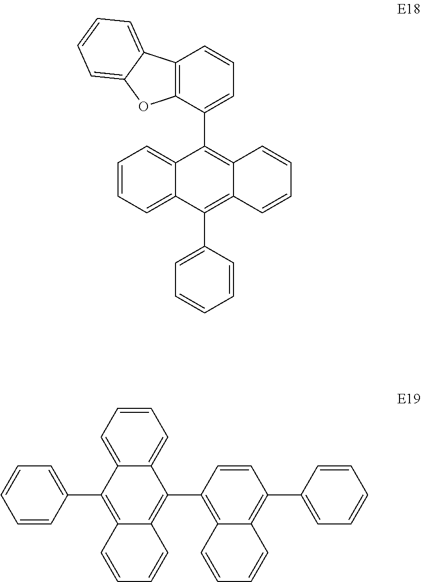

In an embodiment, the light-emitting layer EML may include at least one selected from among a first compound represented by any one selected from among Formulas F-a to F-c, a second compound represented by Formula HT-1, a third compound represented by ET-1, and a fourth compound represented by Formula D-1. In an embodiment, the second compound may be utilized as a hole transporting host material of the light-emitting layer EML.

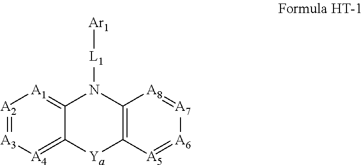

In Formula HT-1, A₁ to A₈ may each independently be N or CR₅₁. For example, all A₁ to A₈ may be CR₅₁. In some embodiments, any one selected from among A₁ to A₈ may be N, and the rest may be CR₅₁.

In Formula HT-1, L₁ may be a direct linkage, a substituted or unsubstituted arylene group having 6 to 30 ring-forming carbon atoms, or a substituted or unsubstituted heteroarylene group having 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, etc. However, an embodiment of the present disclosure is not limited thereto.

In Formula HT-1, Yₐ may be a direct linkage, CR₅₂R₅₃, or SiR₅₄R₅₅. For example, it may refer to that two benzene rings connected with a nitrogen atom in Formula HT-1 are connected via a direct linkage,

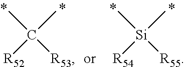

In Formula HT-1, when Yₐ is a direct linkage, the second compound represented by Formula HT-1 may include a carbazole moiety.

In Formula HT-1, Ar₁ may be a substituted or unsubstituted aryl group having 6 to 30 ring-forming carbon atoms, or a substituted or unsubstituted heteroaryl group having 2 to 30 ring-forming carbon atoms. For example, Ar₁ may be a substituted or unsubstituted carbazole group, a substituted or unsubstituted dibenzofuran group, a substituted or unsubstituted dibenzothiophene group, a substituted or unsubstituted biphenyl group, etc. However, an embodiment of the present disclosure is not limited thereto.

In Formula HT-1, R₅₁ to R₅₅ may each independently be, a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 ring-forming carbon atoms, a substituted or unsubstituted aryl group having 6 to 60 ring-forming carbon atoms, or a substituted or unsubstituted heteroaryl group having 2 to 60 ring-forming carbon atoms. In some embodiments, R₅₁ to R₅₅ may each be bonded to an adjacent group to form a ring. For example. R₅₁ to R₅₅ may each independently be a hydrogen atom or a deuterium atom. R₅₁ to R₅₅ may each independently be an unsubstituted methyl group, or an unsubstituted phenyl group.

In an embodiment, the second compound represented by Formula HT-1 may be represented by any one selected from among compounds presented in Compound Group 2. The light-emitting layer EML may include at least one selected from among compounds presented in Compound Group 2 as a hole transporting host material.

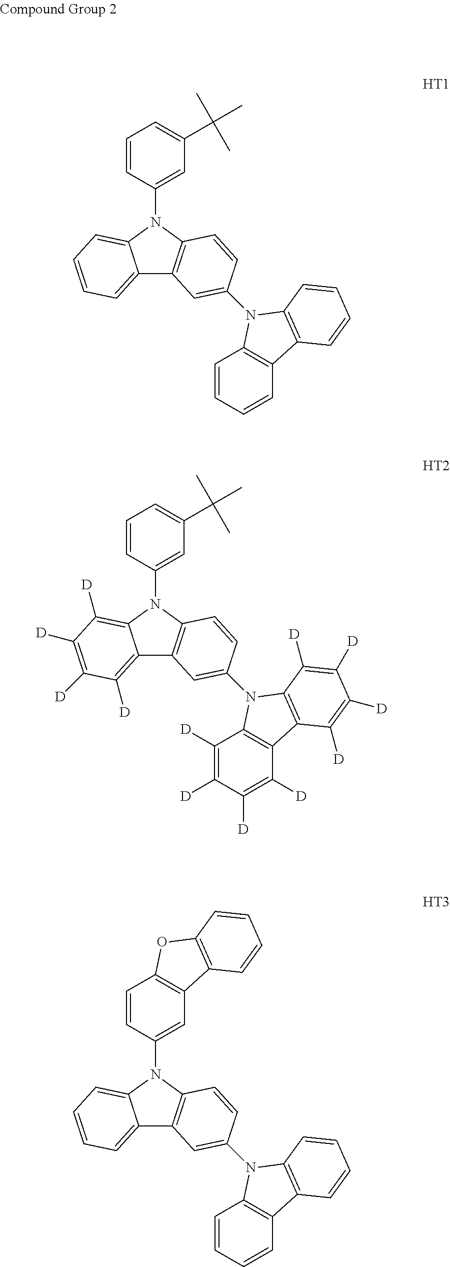 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

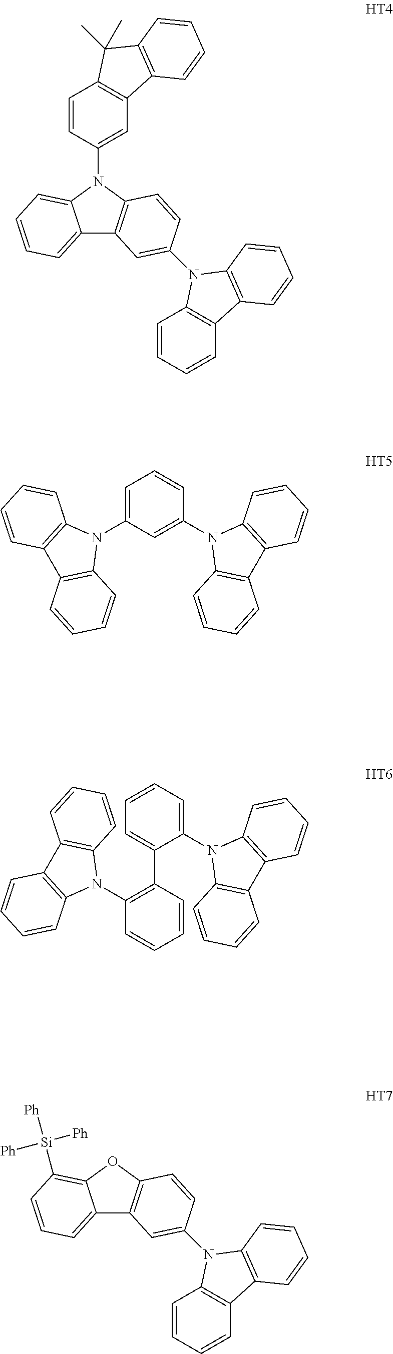 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

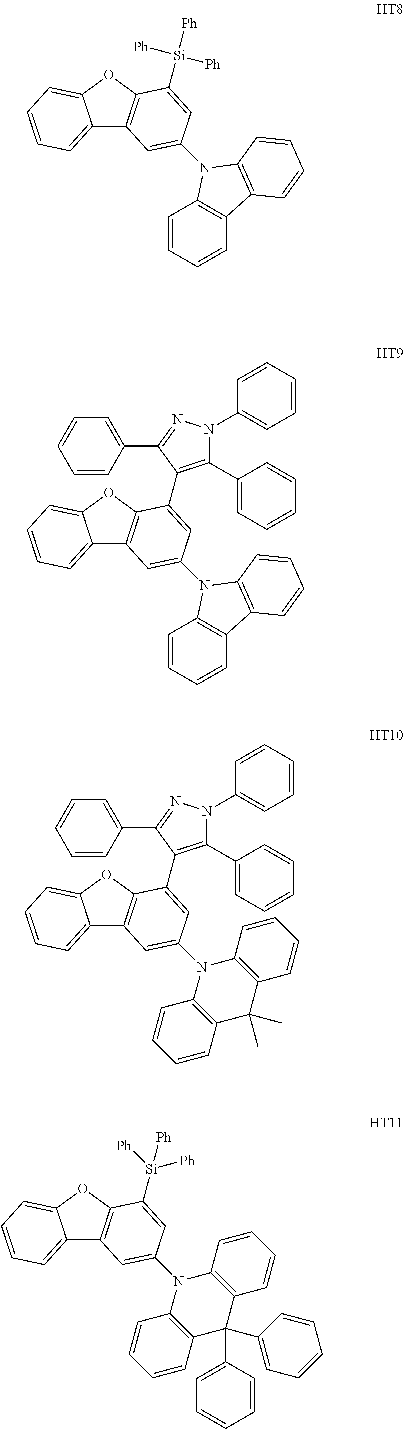 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

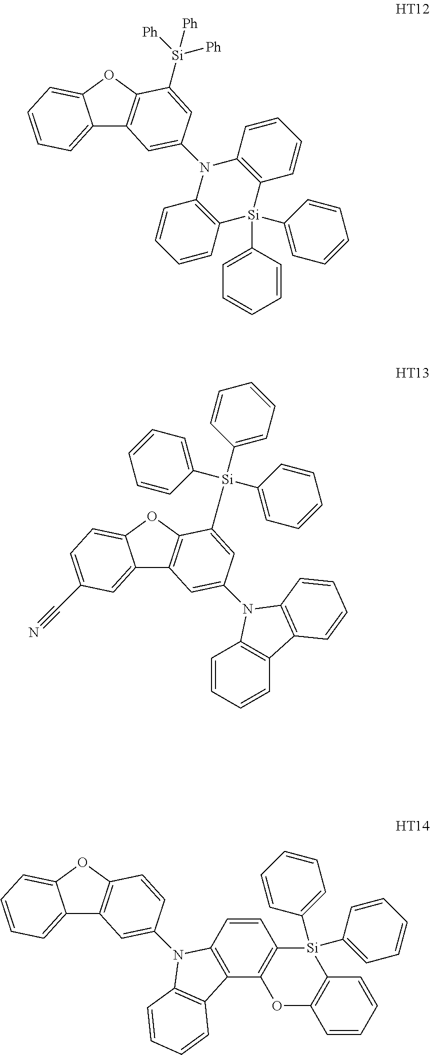 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1]

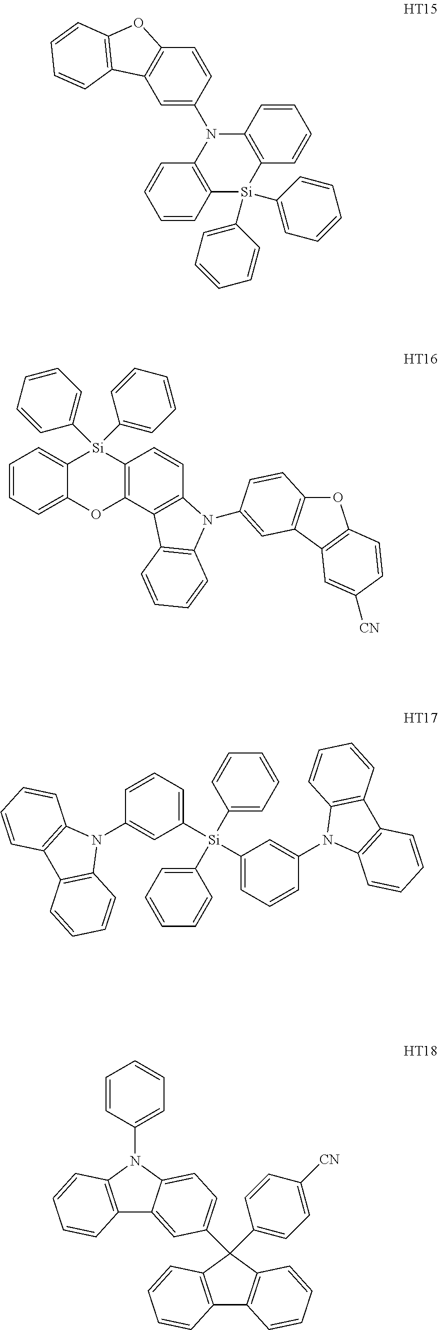 [SMILES: c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1]

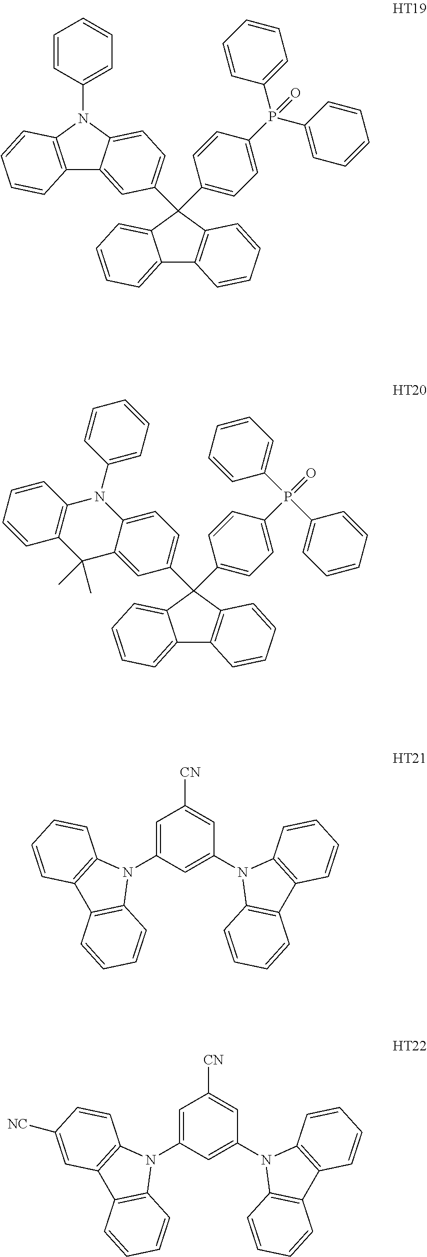 [SMILES: O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1]

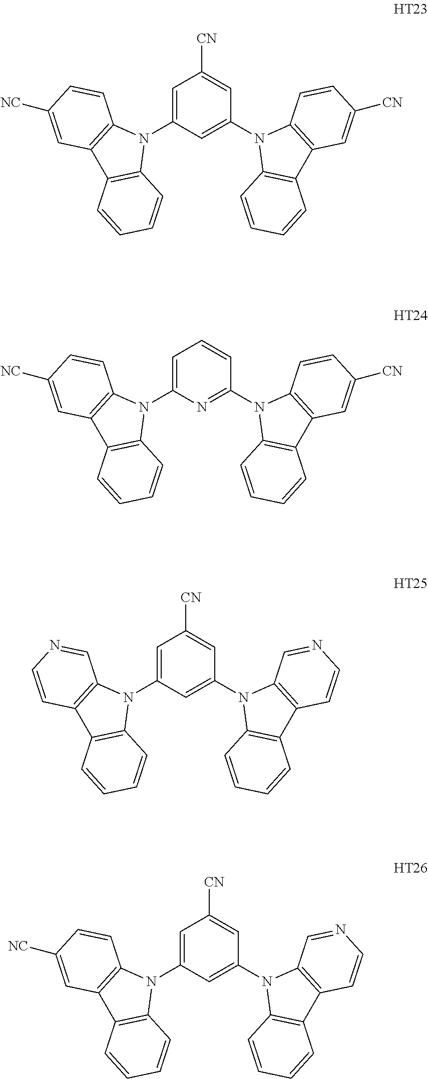 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

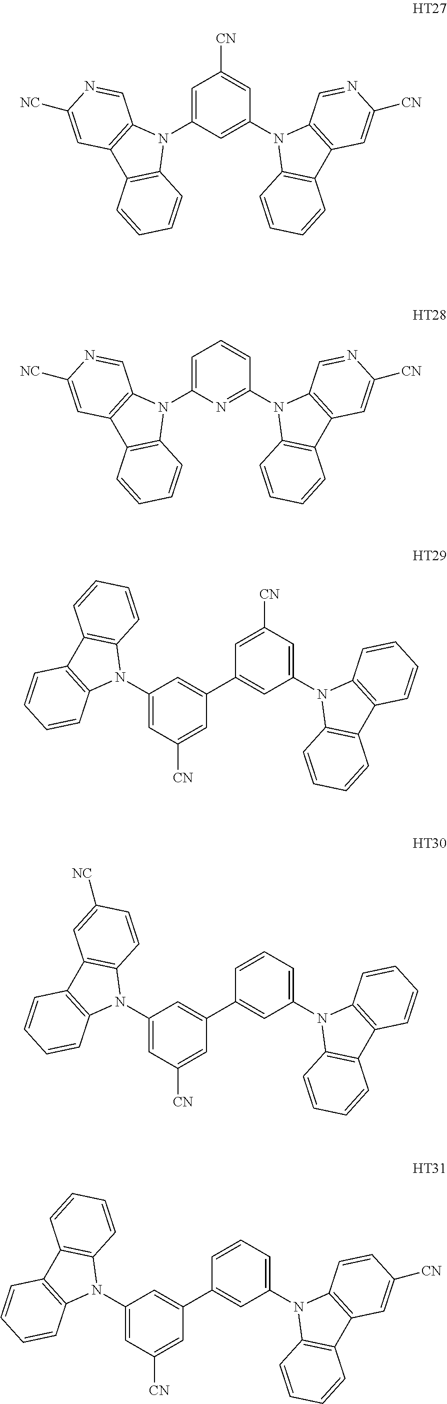 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

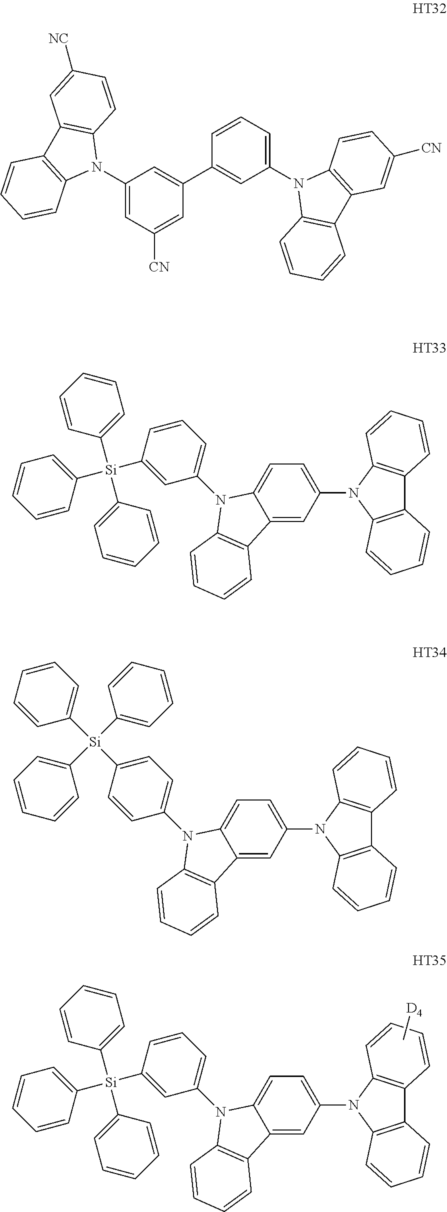

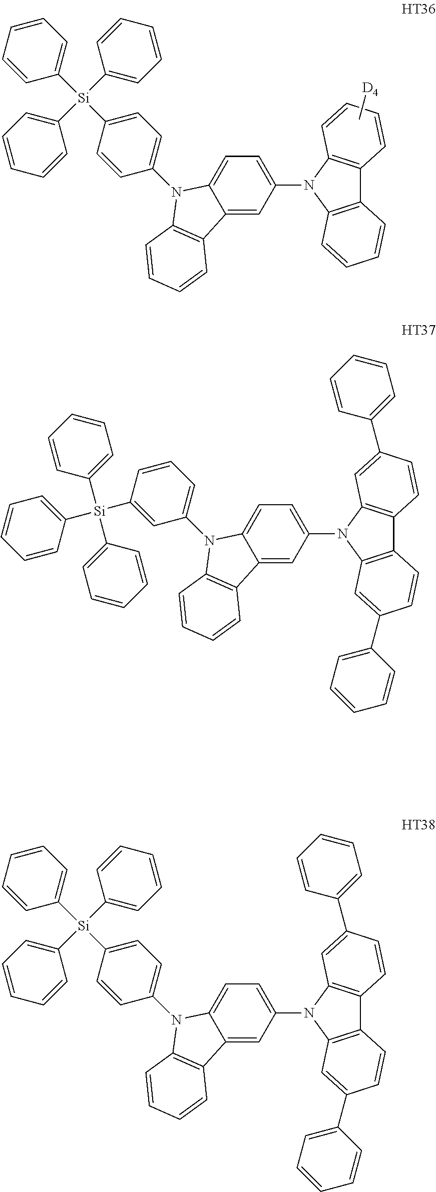

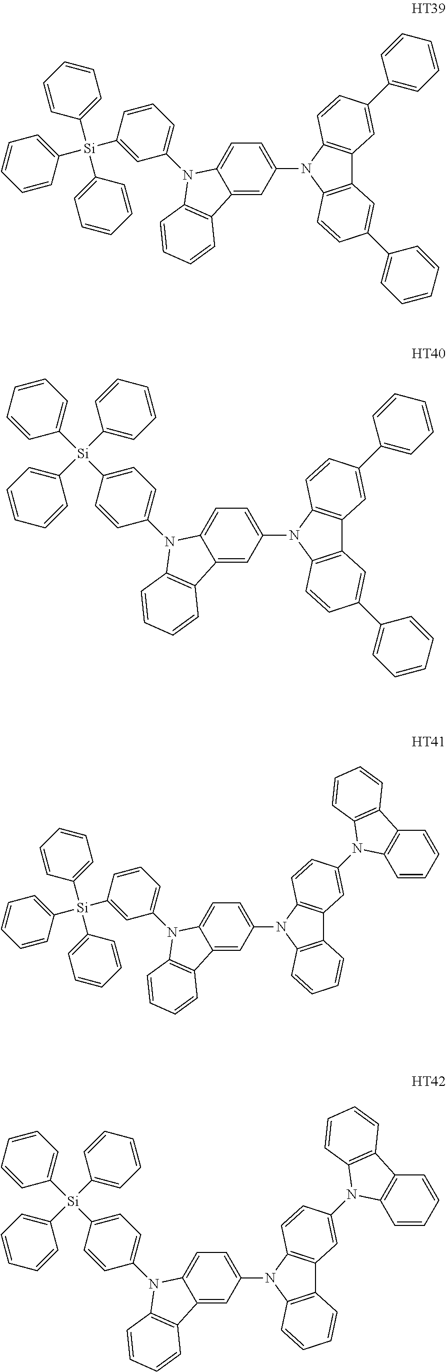 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

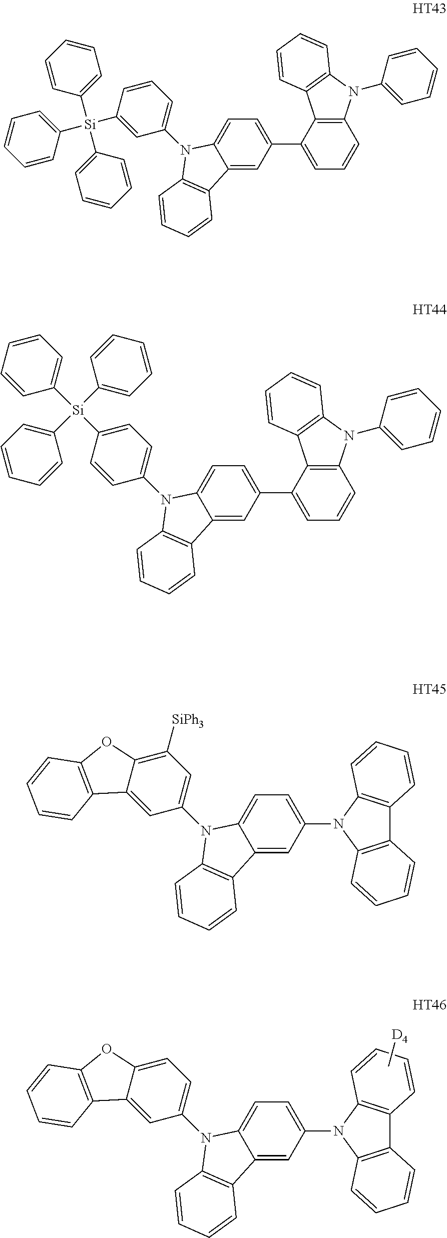

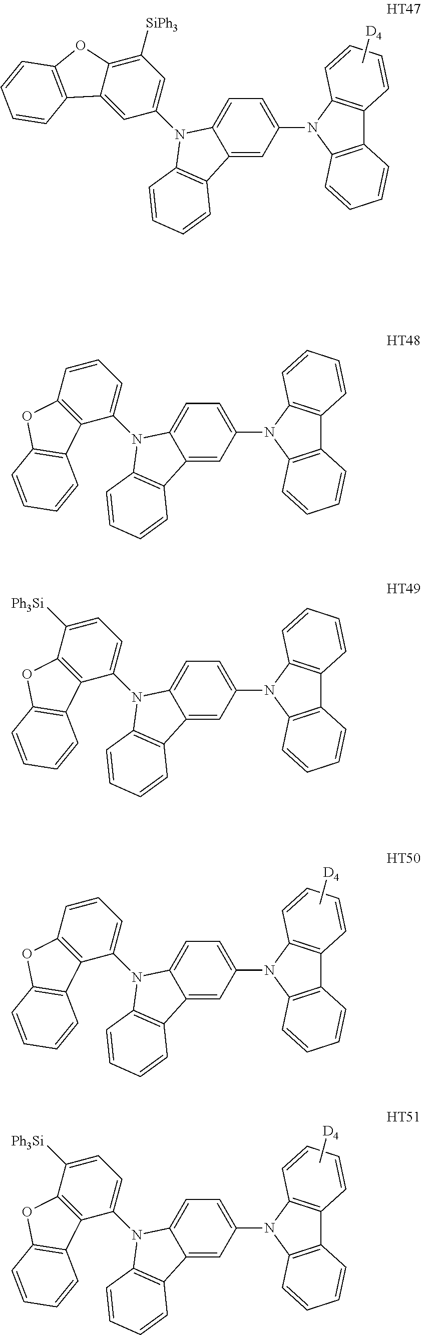

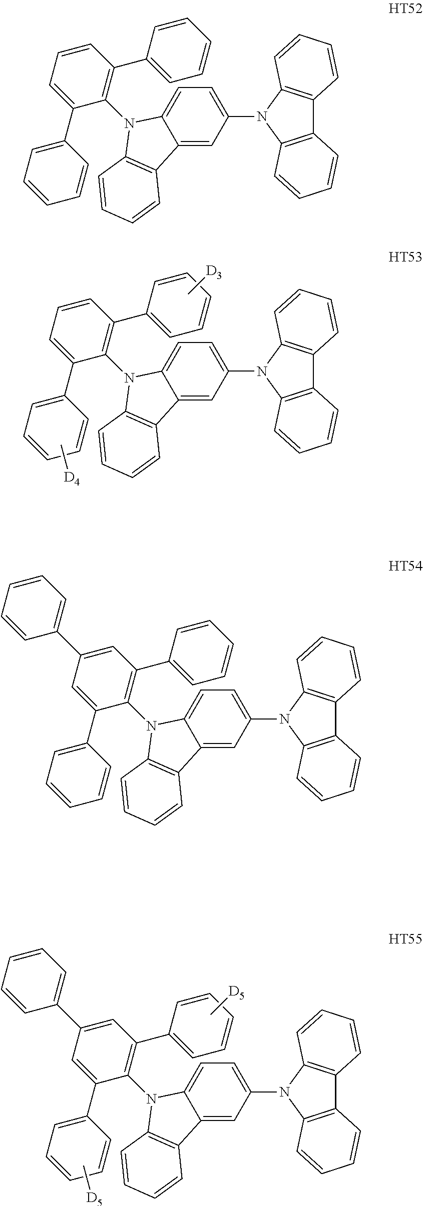

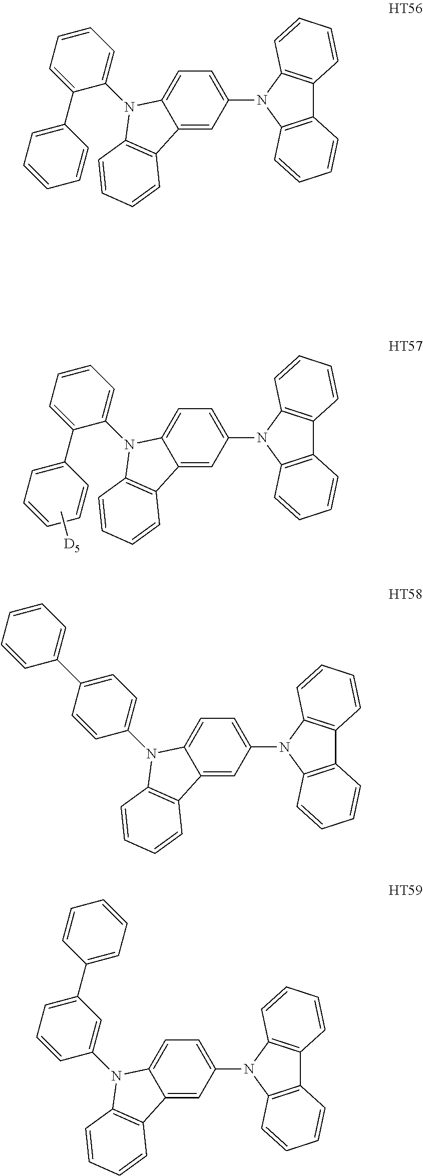

In specific example compounds presented in Compound Group 2, “D” may refer to a deuterium atom, and “Ph” may refer to a substituted or unsubstituted phenyl group. For example, in specific example compounds presented in Compound Group 2, “Ph” may refer to an unsubstituted phenyl group.

In an embodiment, the light-emitting layer EML may include the third compound represented by Formula ET-1. For example, the third compound may be utilized as an electron-transporting host material of the light-emitting layer EML.

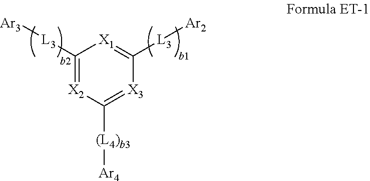

In Formula ET-1, at least one selected from among X₁ to X₃ is N, and the rest is CR₅₆. For example, any one selected from among X₁ to X₃ may be N, and the rest two may each independently be CR₅₆. In this case, the third compound represented by Formula ET-1 may contain a pyridine moiety. In some embodiments, two among X₁ to X₃ may be N, and the other one may be CR₅₆. In this case, the third compound represented by Formula ET-1 may contain a pyrimidine moiety. In some embodiments, all X₁ to X₃ may be N. In this case, the third compound represented by Formula ET-1 may contain a triazine moiety.

In Formula ET-1, R₅₆ may be a hydrogen atom, a deuterium atom, a substituted or unsubstituted alkyl group having 1 to 20 carbon atoms, a substituted or unsubstituted aryl group having 6 to 60 ring-forming carbon atoms, or a substituted or unsubstituted heteroaryl group having 2 to 60 ring-forming carbon atoms.

In Formula ET-1, b1 to b3 may each independently be an integer of 0 to 10.

In Formula ET-1, Ar₂ to Ar₄ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Ar₂ to Ar₄ may be a substituted or unsubstituted phenyl group or a substituted or unsubstituted carbazole group.

In Formula ET-1, L₂ to L₄ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when b1 to b3 are an integer of 2 or more, L₂ to L₄ may each independently be a substituted or unsubstituted arylene group having 6 to 30 ring-forming carbon atoms, or a substituted or unsubstituted heteroarylene group having 2 to 30 ring-forming carbon atoms.

In an embodiment, the third compound may be represented by any one selected from among compounds in Compound Group 3. The light-emitting element ED according to an embodiment may include any one selected from among compounds in Compound Group 3.

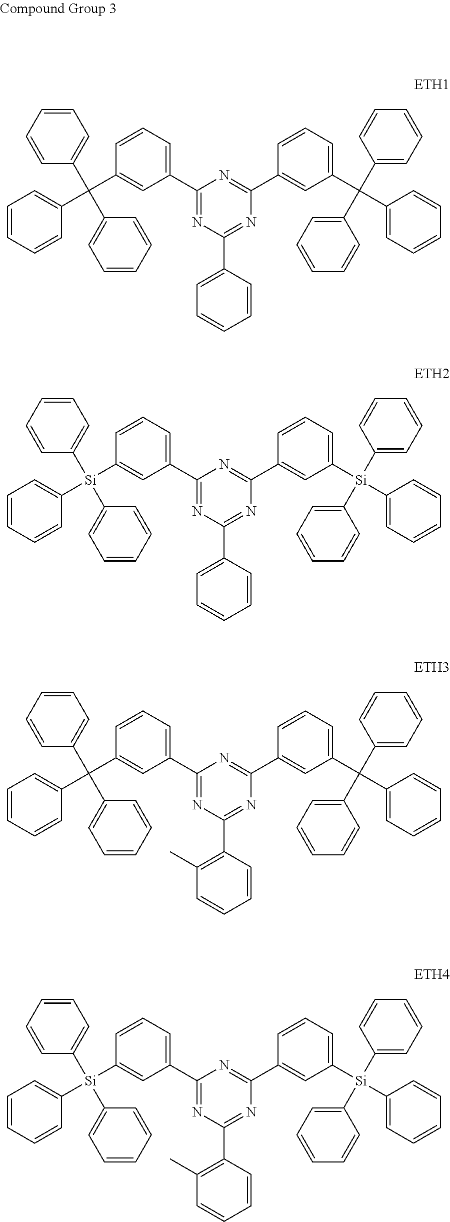 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

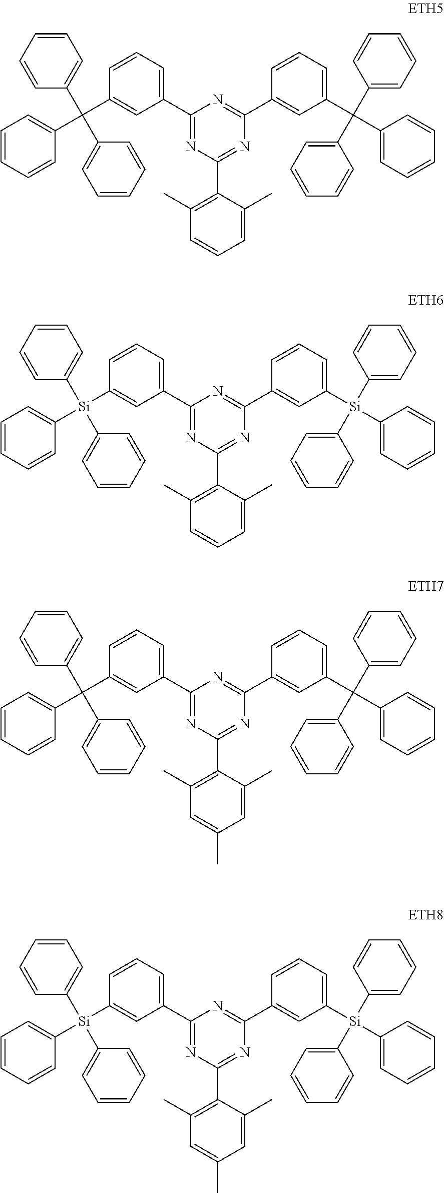 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

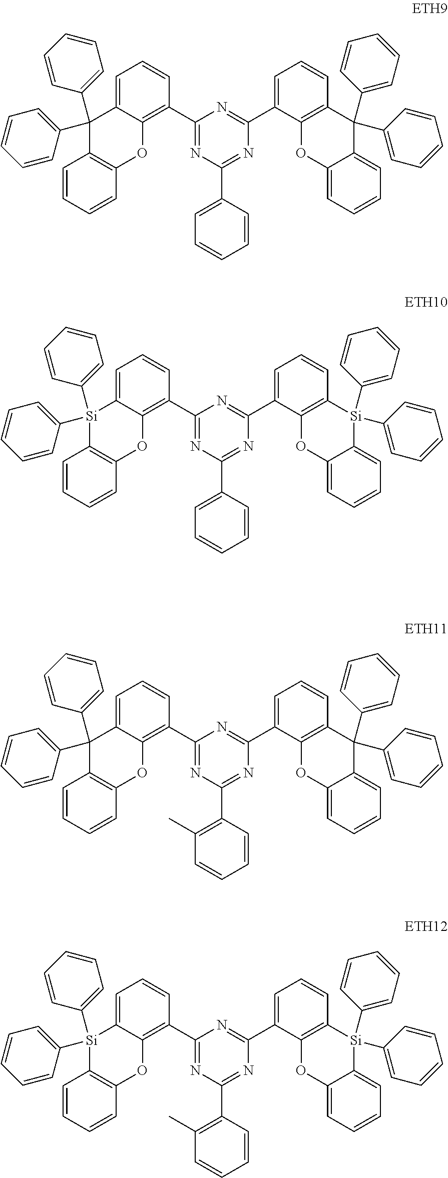 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

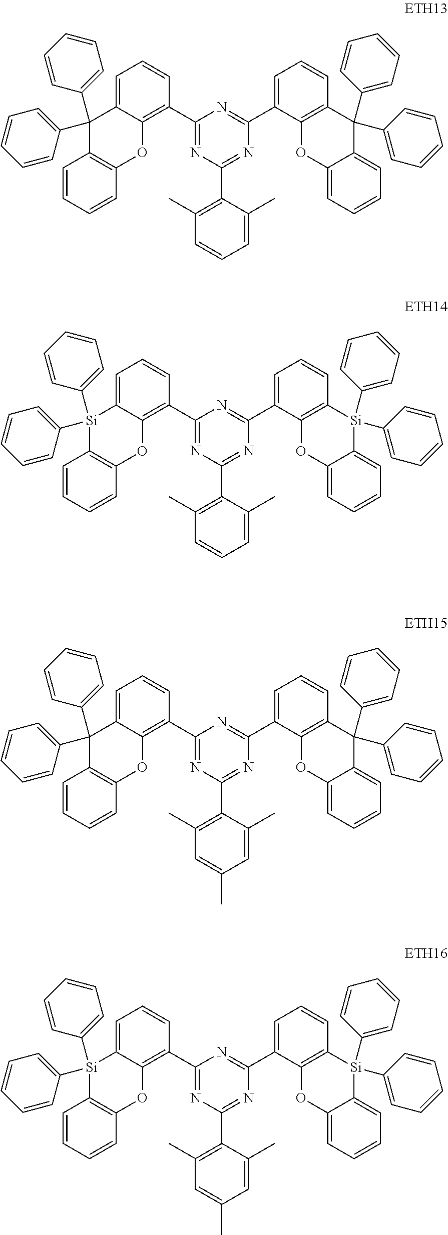 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

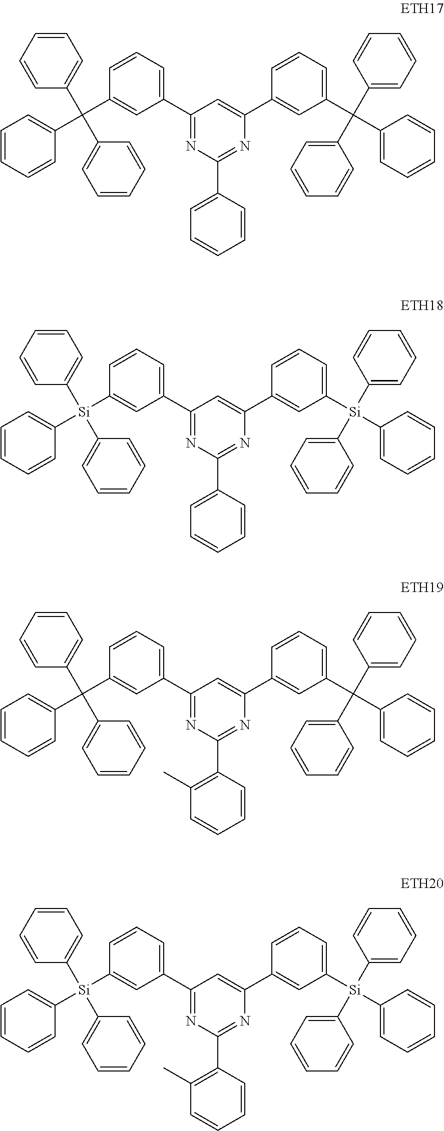 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

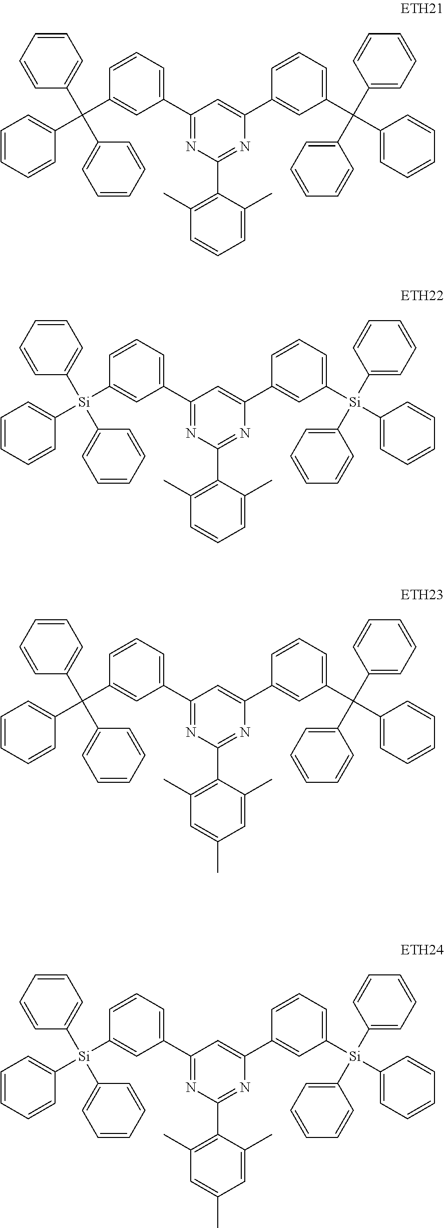 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

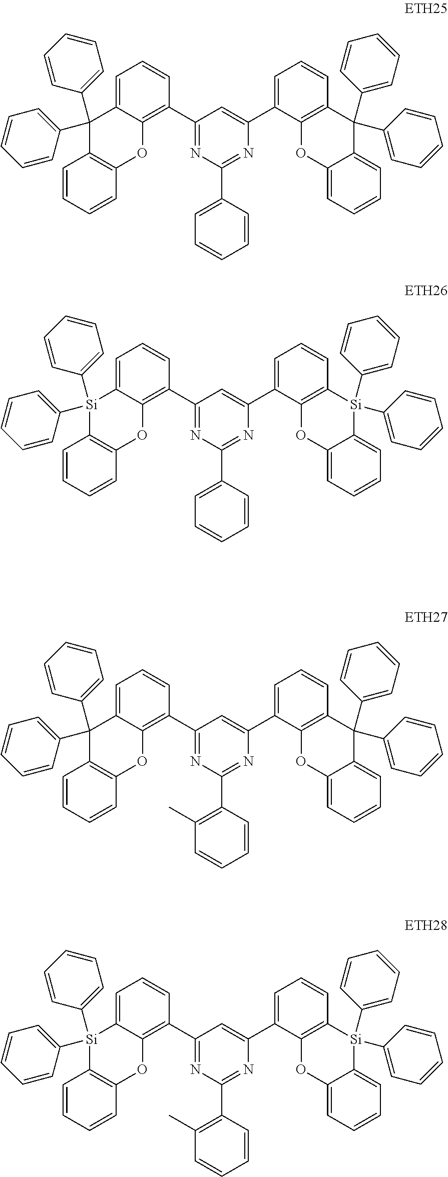 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

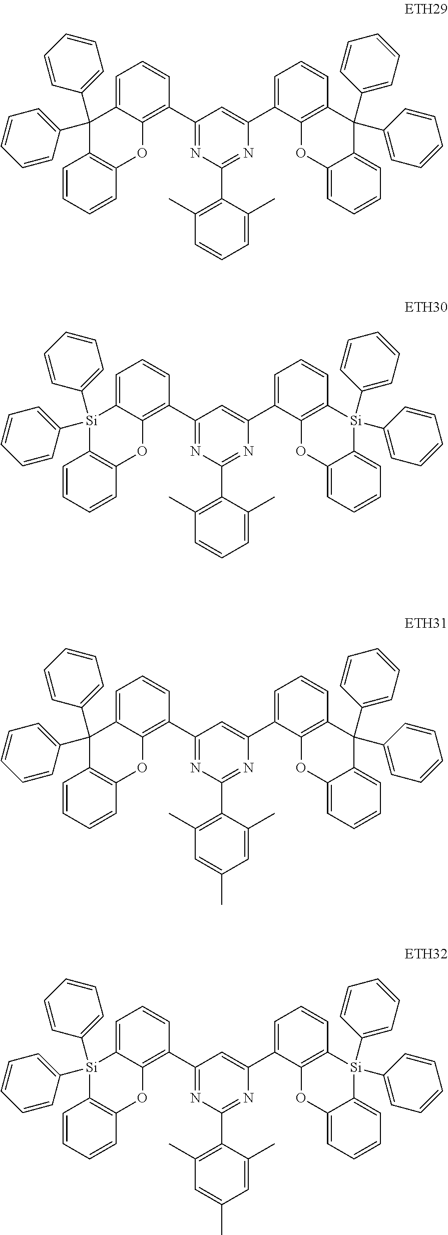 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

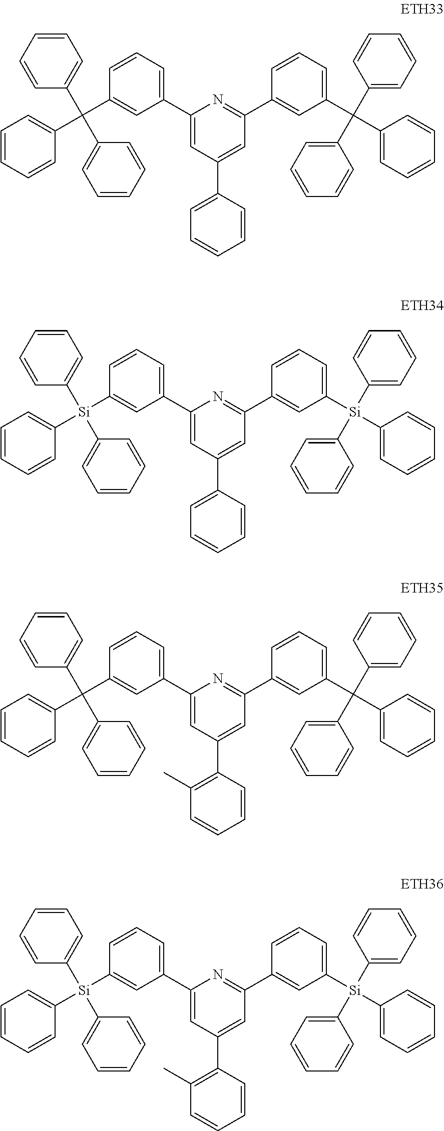 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

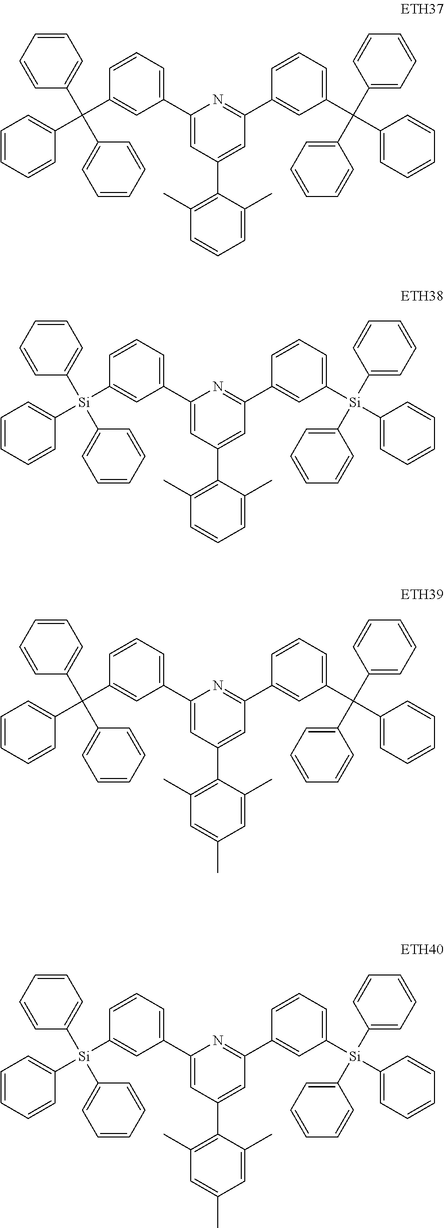 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

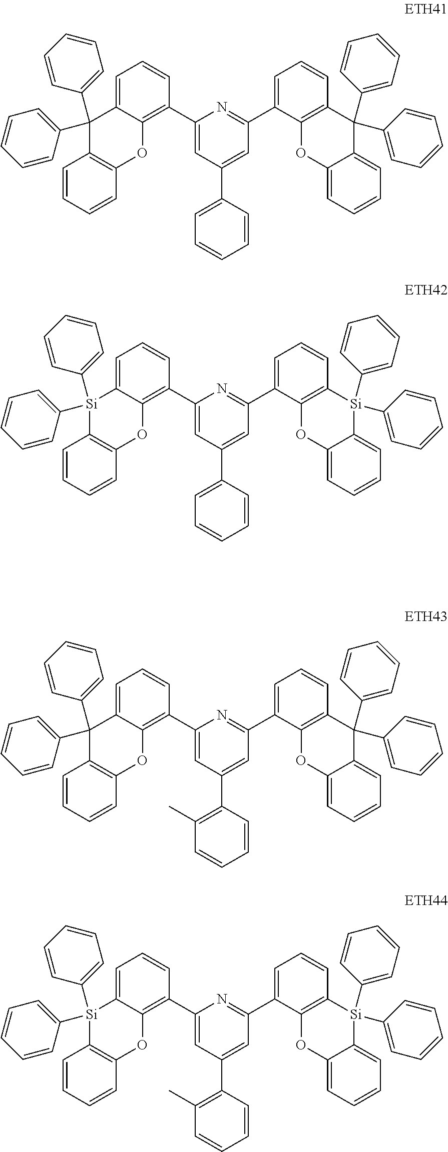 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

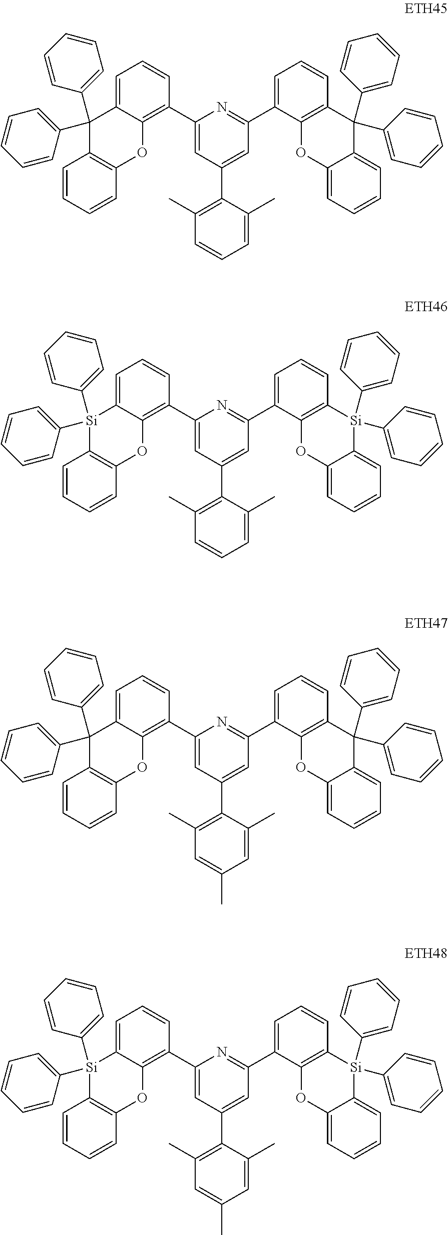 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

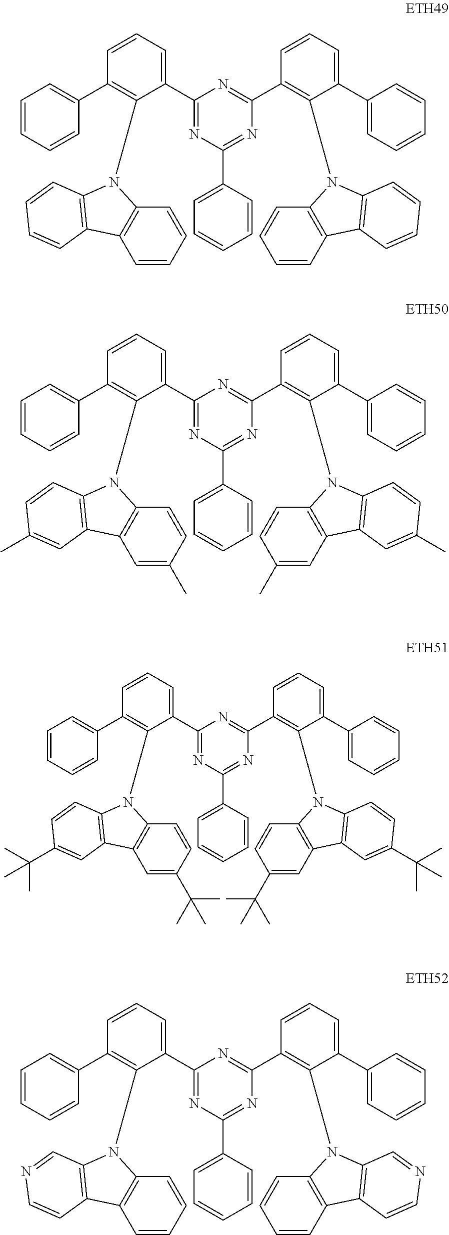

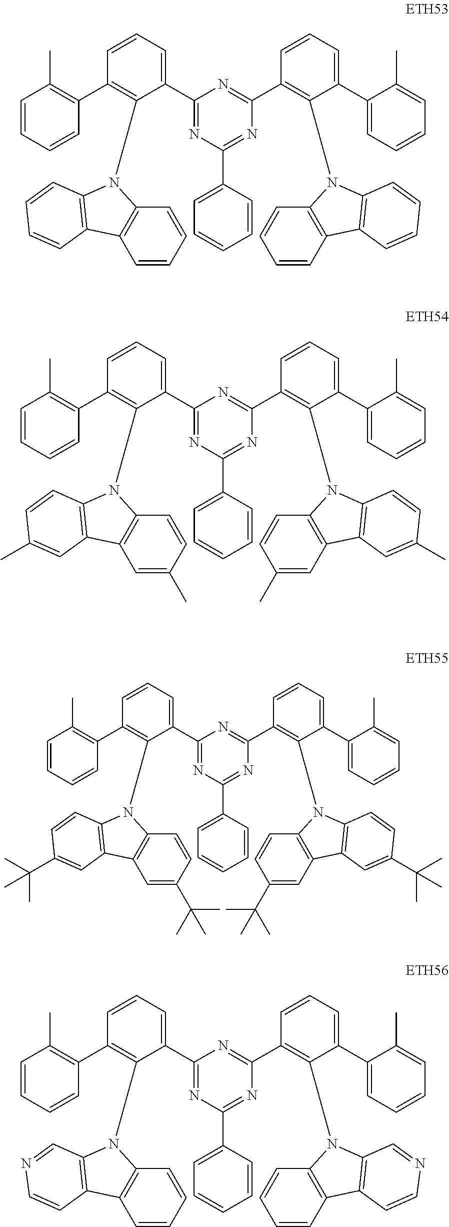

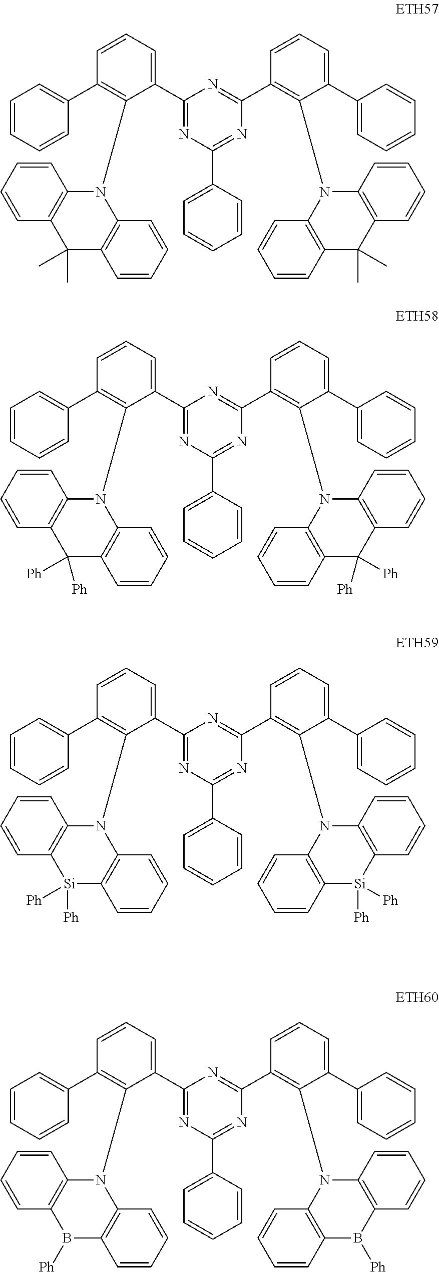

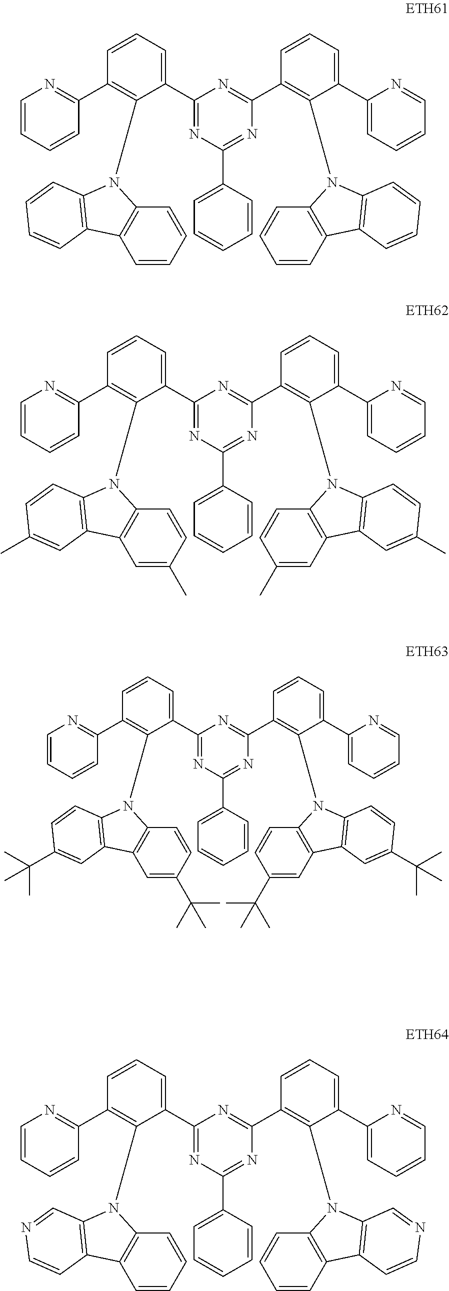

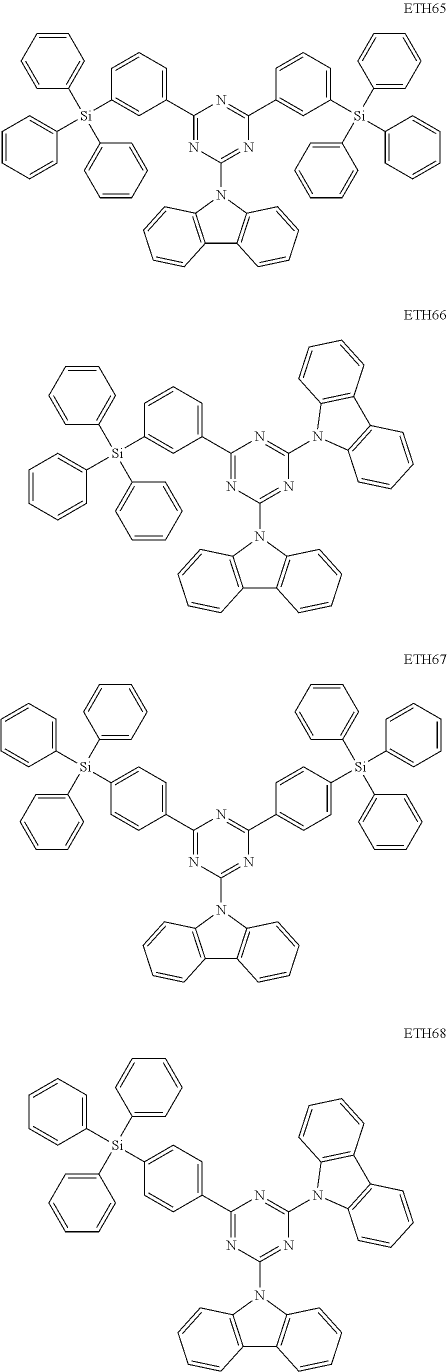 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

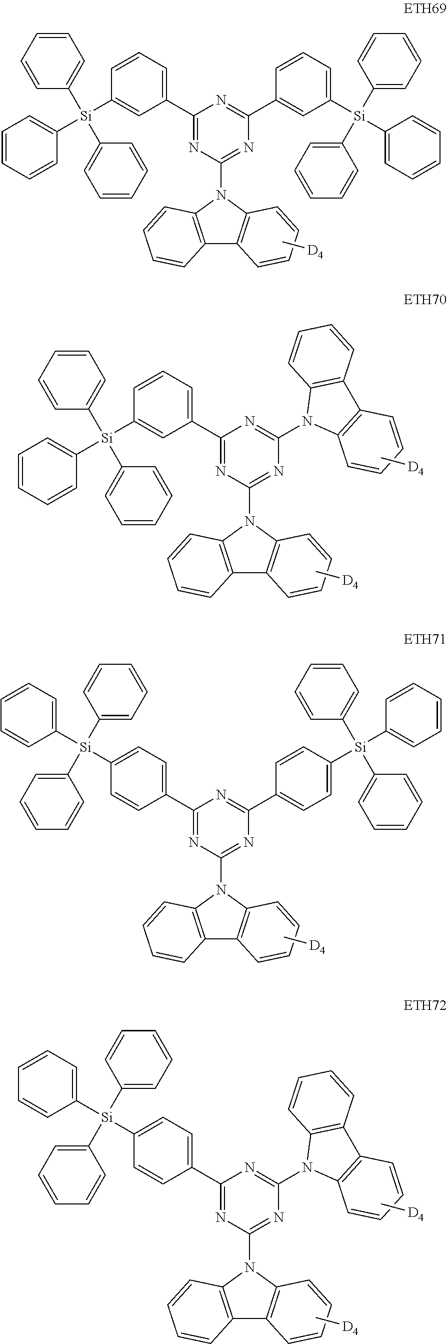

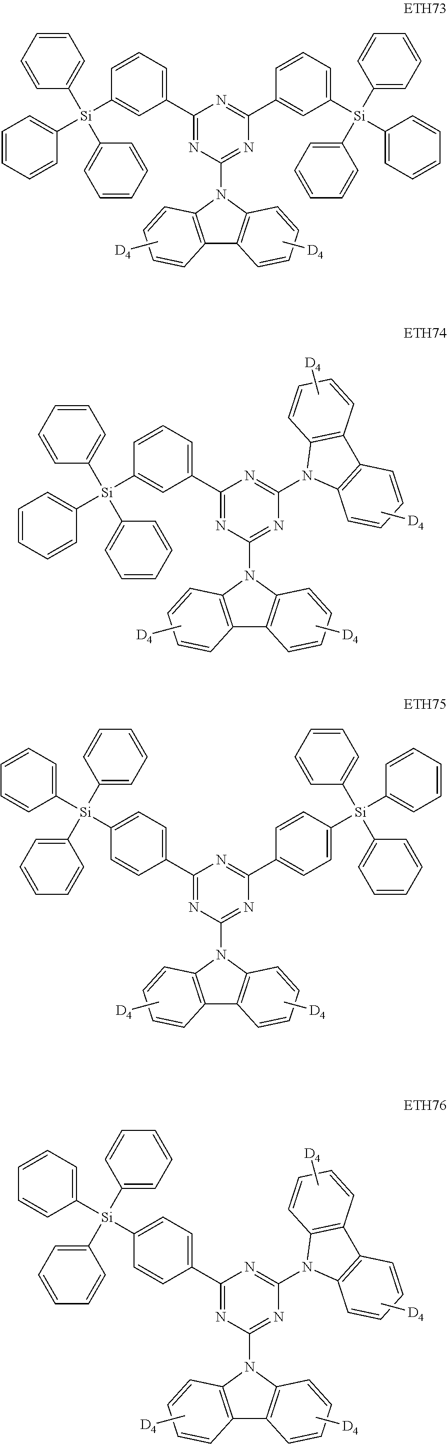

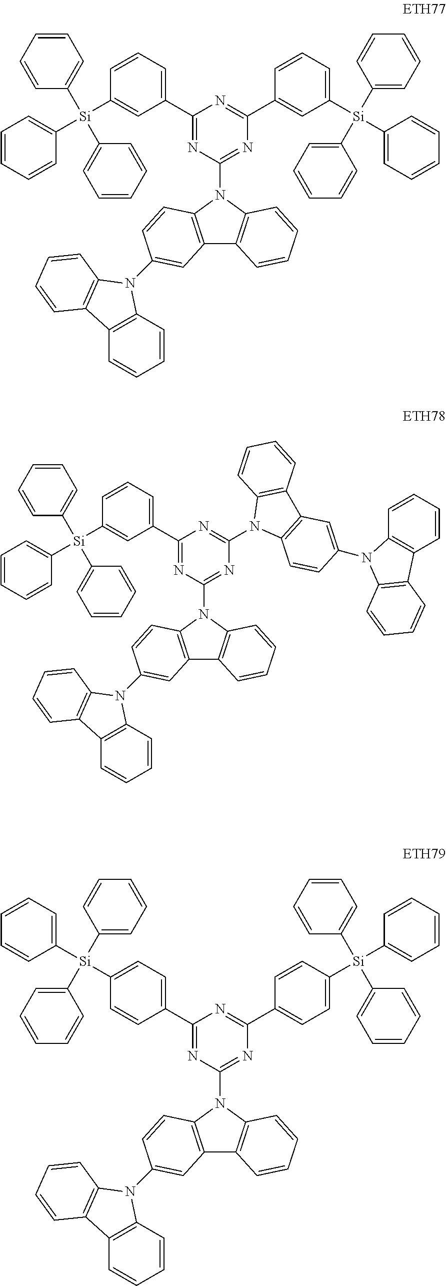 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

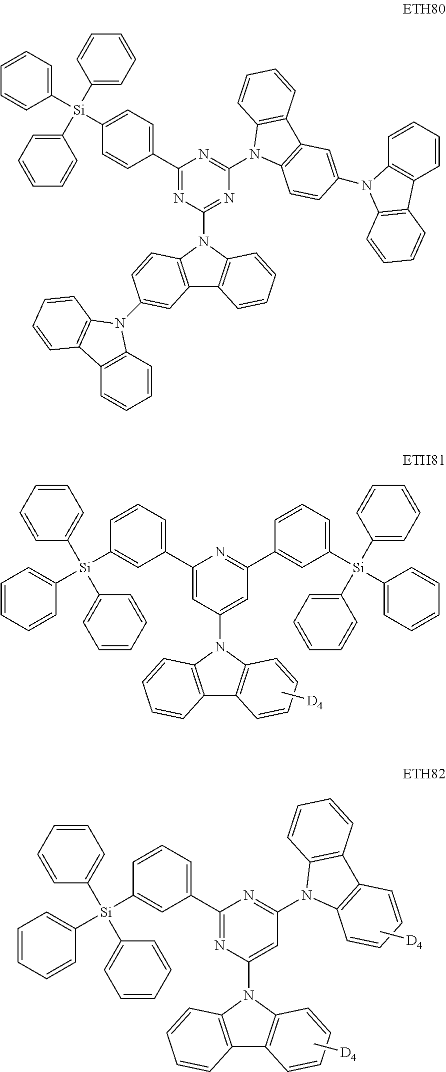

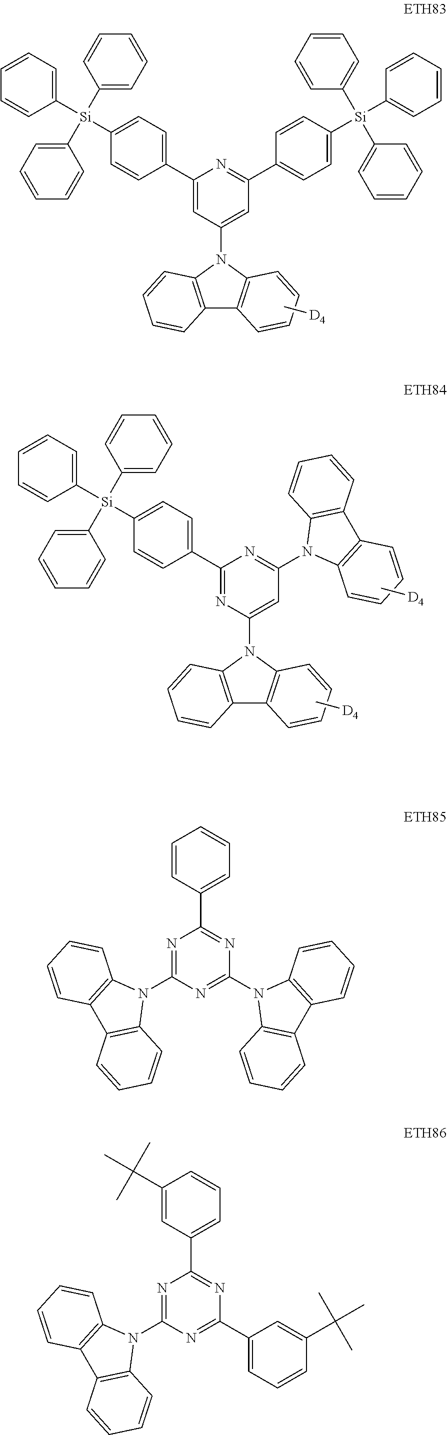

In specific example compounds presented in Compound Group 3, “D” refers to a deuterium atom, and “Ph” refers to an unsubstituted phenyl group.

The emission layer EML may include the second compound and the third compound, and the second compound and the third compound may form an exciplex. In the emission layer EML, an exciplex may be formed by the hole transport host and the electron transport host. In this case, a triplet energy of the exciplex formed by the hole transporting host and the electron transporting host may correspond to the difference between a lowest unoccupied molecular orbital (LUMO) energy level of the electron transporting host and a highest occupied molecular orbital (HOMO) energy level of the hole transporting host.

For example, the absolute value of the triplet energy (T1) of the exciplex formed by the hole transporting host and the electron transporting host may be about 2.4 eV to about 3.0 eV. In some embodiments, the triplet energy of the exciplex may be a value smaller than an energy gap of each host material. The exciplex may have a triplet energy of about 3.0 eV or less that is an energy gap between the hole transporting host and the electron transporting host.

In an embodiment, the emission layer EML may include a fourth compound in addition to the first compound to the third compound as described above. The fourth compound may be utilized as a phosphorescent sensitizer of the emission layer EML. The energy may be transferred from the fourth compound to the first compound, thereby emitting light.

For example, the emission layer EML may include, as the fourth compound, an organometallic complex containing platinum (Pt) as a central metal atom and ligands linked to the central metal atom. The emission layer EML in the light emitting device ED of an embodiment may include, as the fourth compound, a compound represented by Formula D-1:

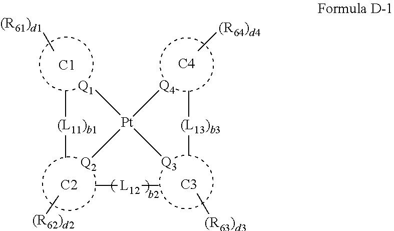

In Formula D-1, Q₁ to Q₄ may each independently be C or N.

In Formula D-1, C1 to C4 may each independently be a substituted or unsubstituted hydrocarbon ring having 5 to 30 ring-forming carbon atoms, or a substituted or unsubstituted heterocycle having 2 to 30 ring-forming carbon atoms.

In Formula D-1, L₁₁ to L₁₃ may each independently be a direct linkage,

*—O—*, *—S—*,

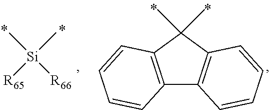

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms. In L₁ to L₁₃, “-*” refers to a part linked to C1 to C4.

In Formula D-1, b1 to b3 may each independently be 0 or 1. When b1 is 0, C1 and C2 may not be linked to each other. When b2 is 0, C2 and C3 may not be linked to each other. When b3 is 0, C3 and C4 may not be linked to each other.

In Formula D-1, R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In some embodiments, each of R₆₁ to R₆₆ may be bonded to an adjacent group to form a ring. R₆₁ to R₆₆ may each independently be a substituted or unsubstituted methyl group, or a substituted or unsubstituted t-butyl group.

In Formula D-1, d1 to d4 may each independently be an integer of 0 to 4. In Formula D-1, when each of d1 to d4 is 0, the fourth compound may not be substituted with each of R₆₁ to R₆₄. The case where each of d1 to d4 is 4 and R₆₁′s to R₆₄′ are each hydrogen atoms may be the same as the case where each of d1 to d4 is 0. When each of d1 to d4 is an integer of 2 or more, a plurality of R₆₁′s to R₆₄′s may each be the same or at least one selected from among the plurality of R₆₁′s to R₆₄′s may be different from the others.

In Formula D-1, C1 to C4 may each independently be a substituted or unsubstituted hydrocarbon ring or a substituted or unsubstituted heterocycle represented by any one selected from among C-1 to C-4:

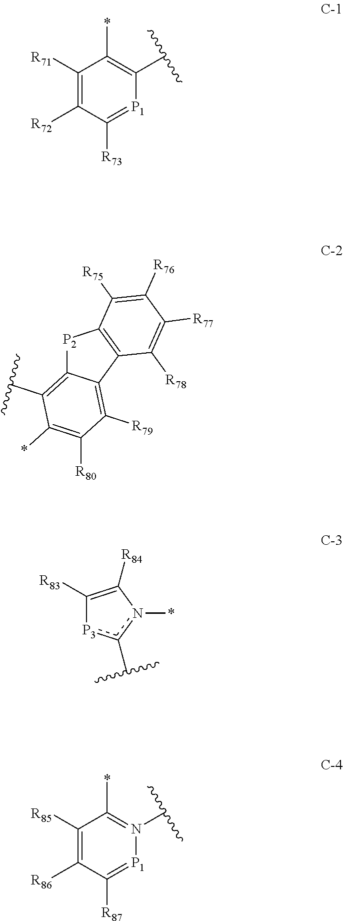

In C-1 to C-4, P₁ may be C—* or CR₇₄, P₂ may be N—* or NR₈₁, P₃ may be N—* or NR₈₂, and P₄ may be C—* or CR₈₈. R₇₁ to R₈₈ may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In some embodiments, in C-1 to C-4,

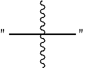

corresponds to a part linked to Pt that is a central metal atom, and “-*” corresponds to a part linked to a neighboring cyclic group (C1 to C4) or a linker (L₁₁ to L₁₃).

The emission layer EML of an embodiment may include the first compound, which is a fused polycyclic compound, and at least one of the second to fourth compounds. For example, the emission layer EML may include the first compound, the second compound, and the third compound. In the emission layer EML, the second compound and the third compound may form an exciplex, and the energy may be transferred from the exciplex to the first compound, thereby emitting light.

In some embodiments, the emission layer EML may include the first compound, the second compound, the third compound, and the fourth compound. In the emission layer EML, the second compound and the third compound may form an exciplex, and the energy may be transferred from the exciplex to the fourth compound and the first compound, thereby emitting light. In an embodiment, the fourth compound may be a sensitizer. The fourth compound included in the emission layer EML in the light emitting device ED of an embodiment may serve as a sensitizer to deliver energy from the host to the first compound that is a light emitting dopant. For example, the fourth compound serving as an auxiliary dopant accelerates energy delivery to the first compound that is a light emitting dopant, thereby increasing the emission ratio of the first compound. Therefore, the emission layer EML of an embodiment may improve luminous efficiency. In some embodiments, when the energy delivery to the first compound is increased, an exciton formed in the emission layer EML is not accumulated inside the emission layer EML and emits light rapidly, and thus deterioration of the device may be reduced. Therefore, the service life of the light emitting device ED of an embodiment may increase.

The light emitting device ED of an embodiment may include all of the first compound, the second compound, the third compound, and the fourth compound, and the emission layer EML may include the combination of two host materials and two dopant materials. In the light emitting device ED of an embodiment, the emission layer EML may concurrently (e.g., simultaneously) include the second compound and the third compound, which are two different hosts, the first compound that is configured to emit a delayed fluorescence, and the fourth compound including an organometallic complex, thereby exhibiting excellent or suitable luminous efficiency characteristics.

In an embodiment, the fourth compound represented by Formula D-1 may represented at least one selected from among the compounds represented by Compound Group 4. The emission layer EML may include at least one selected from among the compounds represented by Compound Group 4 as a sensitizer material.

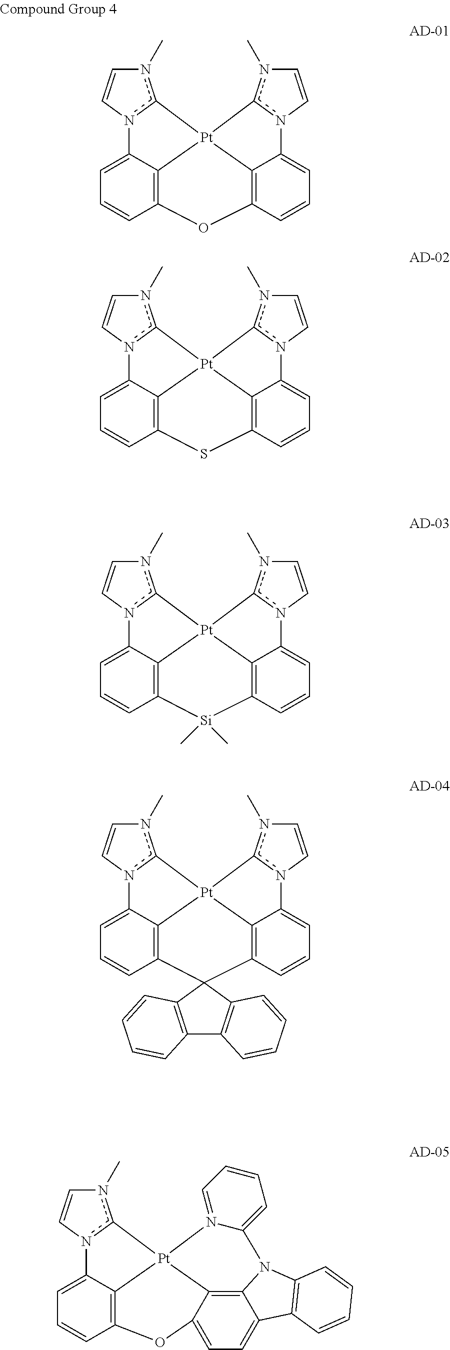

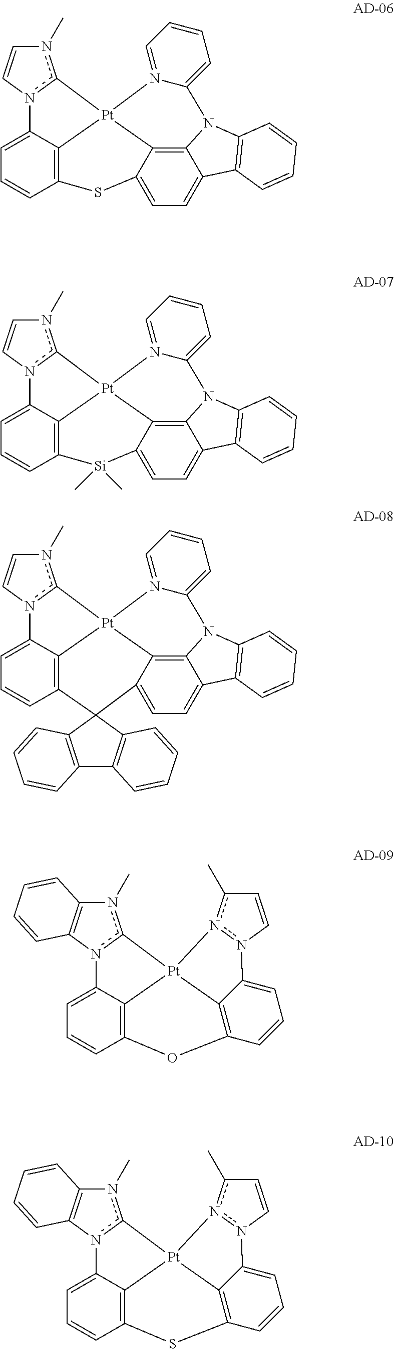

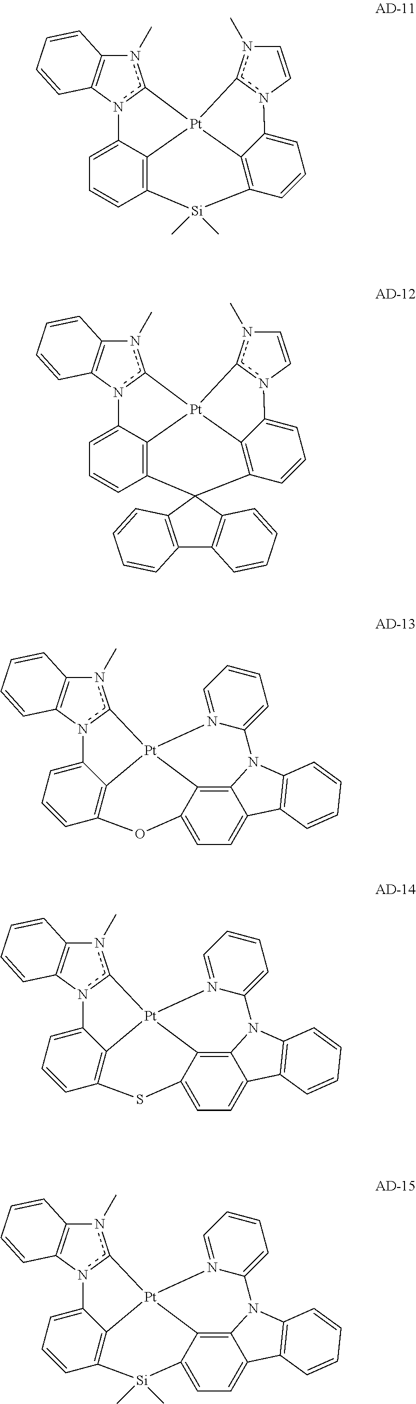

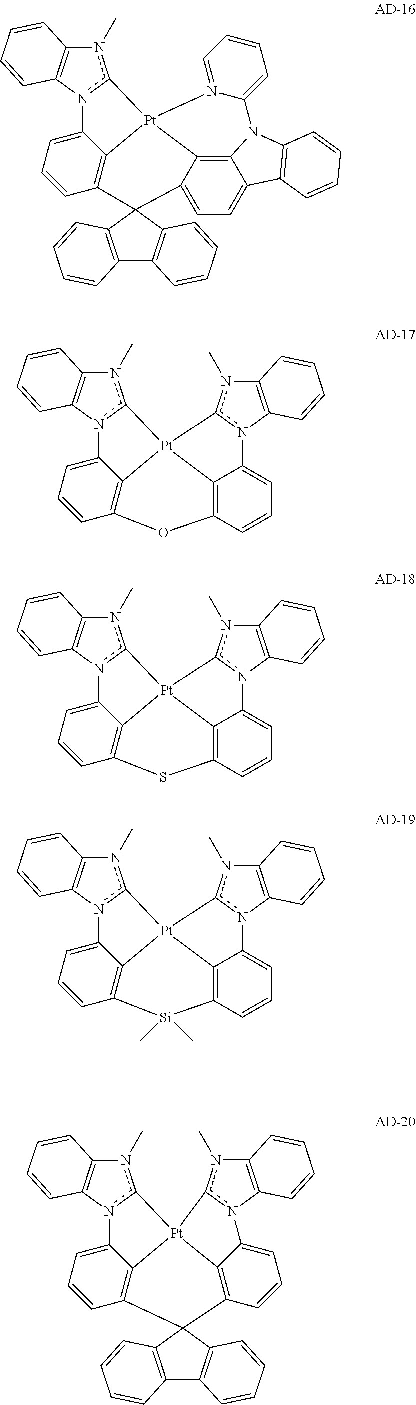

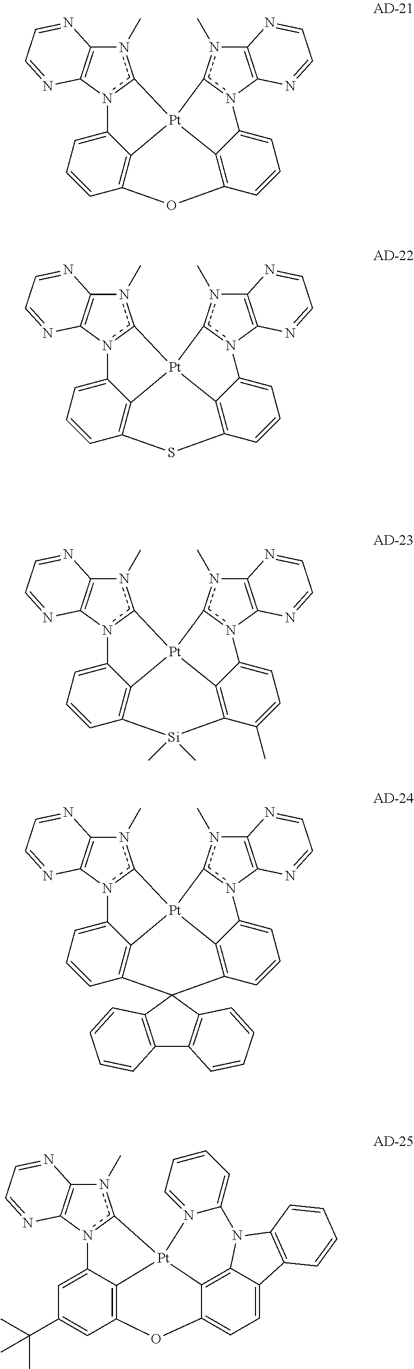

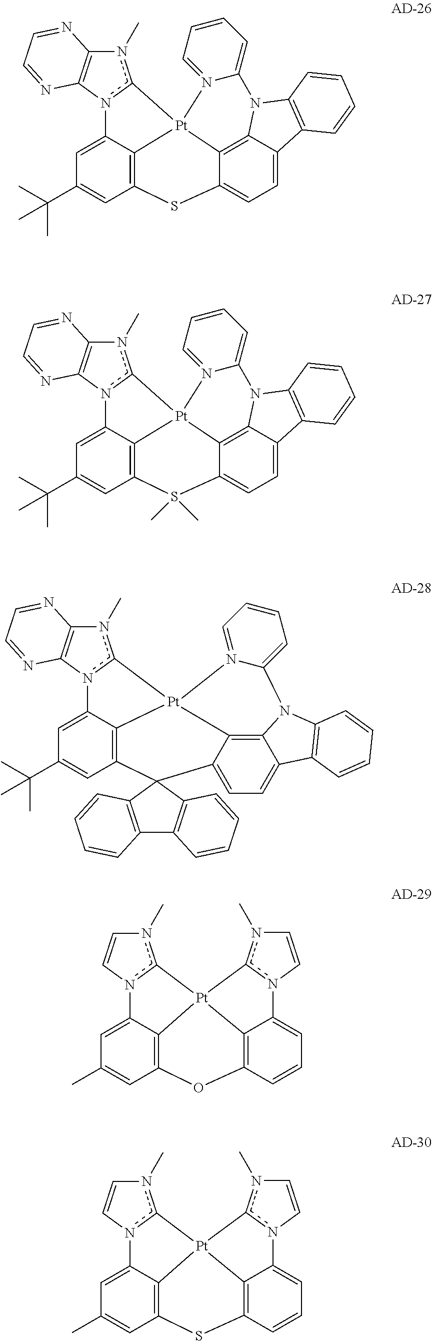

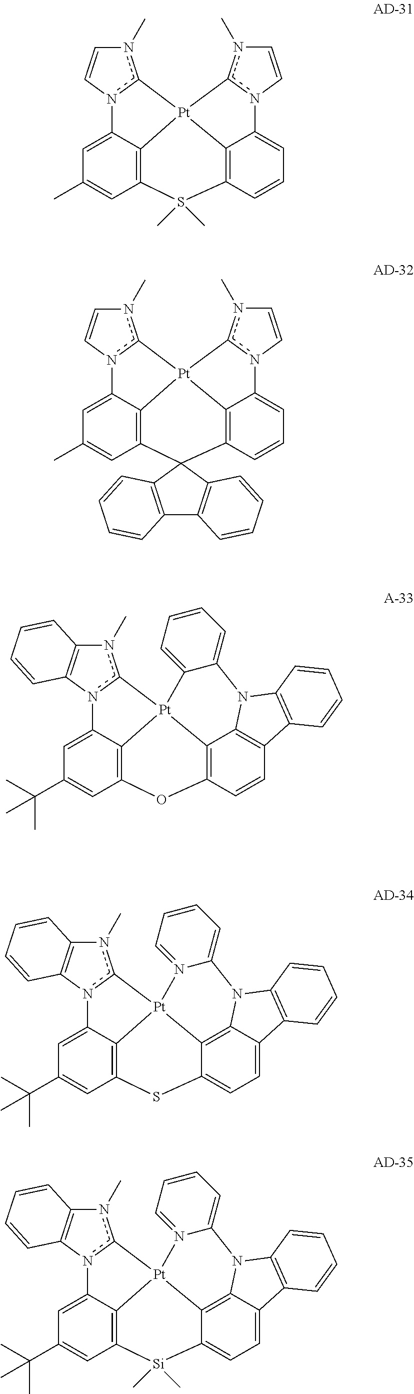

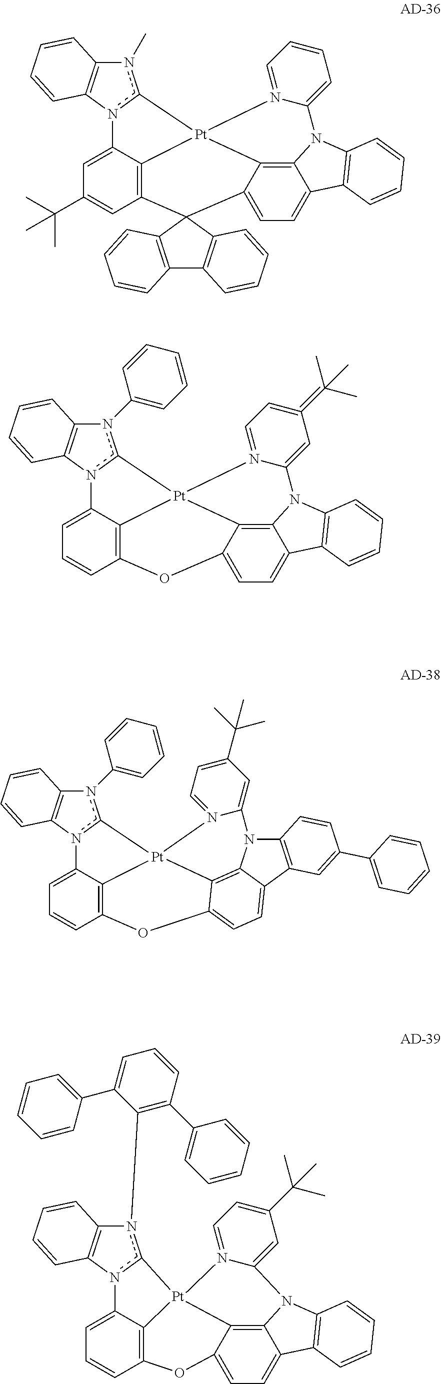

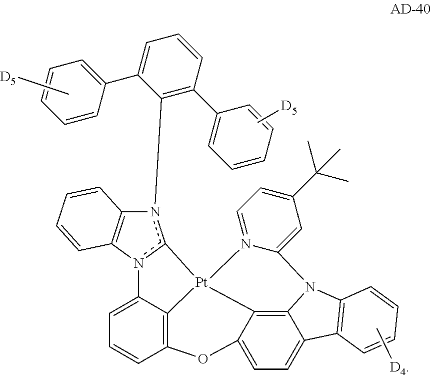

In the embodiment compounds presented in Compound Group 4, “D” refers to a deuterium atom.

When the emission layer EML in the light emitting device ED of an embodiment includes all of the first compound, the second compound, and the third compound, with respect to the total weight of the first compound, the second compound, and the third compound, the content (e.g., amount) of the first compound may be about 0.1 wt % to about 5 wt %. However, the embodiment of the present disclosure is not limited thereto. When the content (e.g., amount) of the first compound satisfies the above-described proportion, the energy transfer from the second compound and the third compound to the first compound may increase, and thus the luminous efficiency and device service life may increase.

The contents of the second compound and the third compound in the emission layer EML may be the rest excluding the weight of the first compound. For example, the contents of the second compound and the third compound in the emission layer EML may be about 65 wt % to about 95 wt % with respect to the total weight of the first compound, the second compound, and the third compound.

In the total weight of the second compound and the third compound, the weight ratio of the second compound and the third compound may be about 3:7 to about 7:3.

When the contents of the second compound and the third compound satisfy the above-described ratio, a charge balance characteristic in the emission layer EML are improved, and thus the luminous efficiency and device service life may increase. When the contents of the second compound and the third compound deviate from the above-described ratio range, a charge balance in the emission layer EML is broken, and thus the luminous efficiency may be reduced and the device may be easily deteriorated.

When the emission layer EML includes the fourth compound, the content (e.g., amount) of the fourth compound in the emission layer EML may be about 10 wt % to about 30 wt % with respect to the total weight of the first compound, the second compound, the third compound, and the fourth compound. However, the embodiment of the present disclosure is not limited thereto. When the content (e.g., amount) of the fourth compound satisfies the above-described content (e.g., amount), the energy delivery from the host to the first compound which is a light emitting dopant may be increased, thereby a luminous ratio may be improved, and thus the luminous efficiency of the emission layer EML may be improved. When the first compound, the second compound, the third compound, and the fourth compound included in the emission layer EML satisfy the above-described content (e.g., amount) ratio range, excellent or suitable luminous efficiency and long service life may be achieved.

In an embodiment, the emission layer EML may include a compound represented by Formula E-2a or Formula E-2b. The compound represented by Formula E-2a or Formula E-2b may be utilized as a phosphorescent host material.

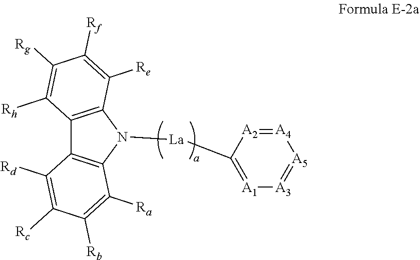

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of Lₐ′s may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, etc., as a ring-forming atom.

In some embodiments, in Formula E-2a, two or three selected from among A₁ to A₅ may be N, and the rest may be CRᵢ.

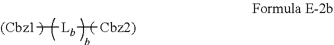

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer of 0 to 10, and when b is an integer of 2 or more, a plurality of L_{b}′s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among the compounds of Compound Group E-2. However, the compounds listed in Compound Group E-2 are examples, and the compound represented by Formula E-2a or Formula E-2b is not limited to those represented in Compound Group E-2.

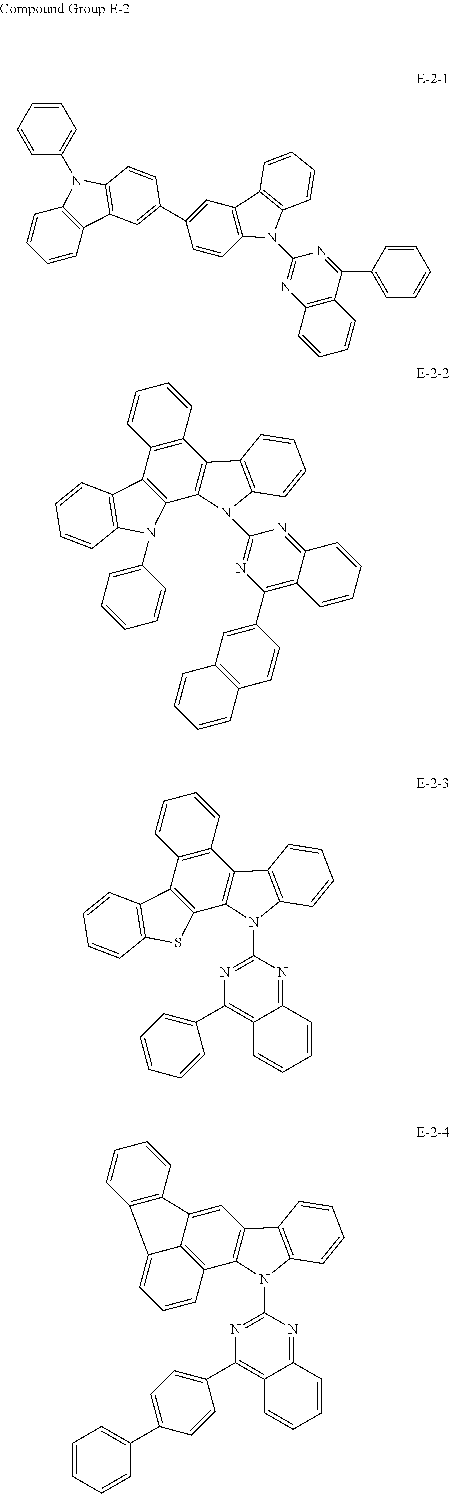 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5cc6c7c(cccc7c54)-c4ccccc4-6)nc4ccccc34)cc2)cc1]

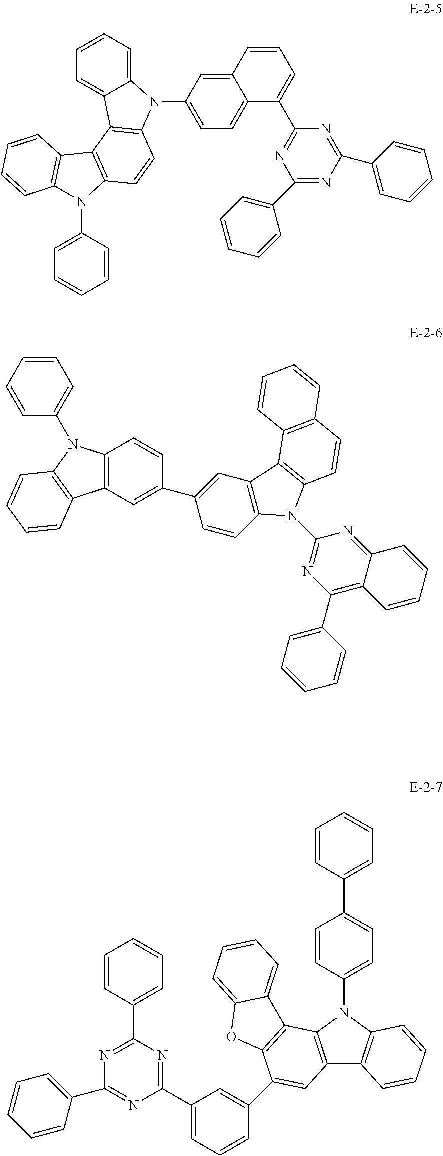 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

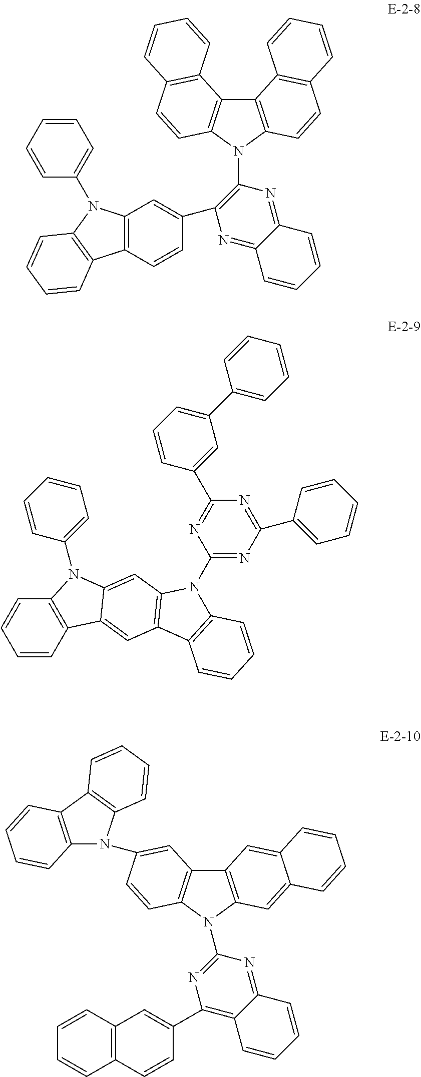 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

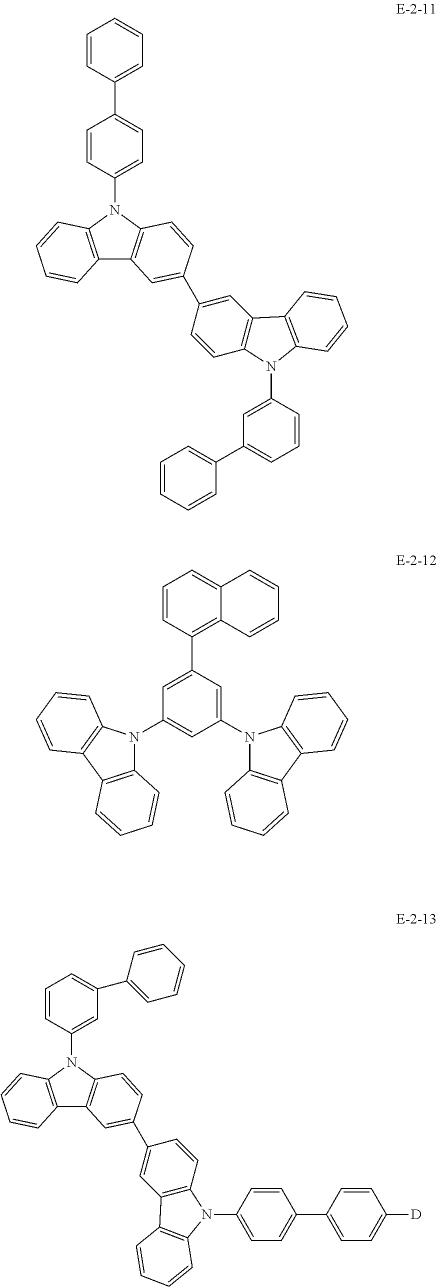 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

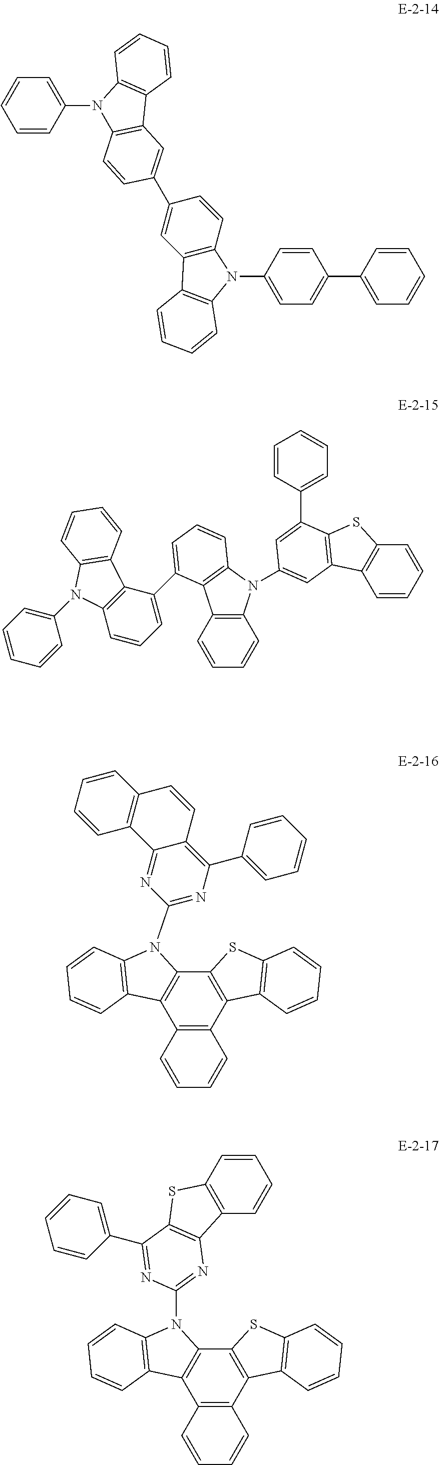 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

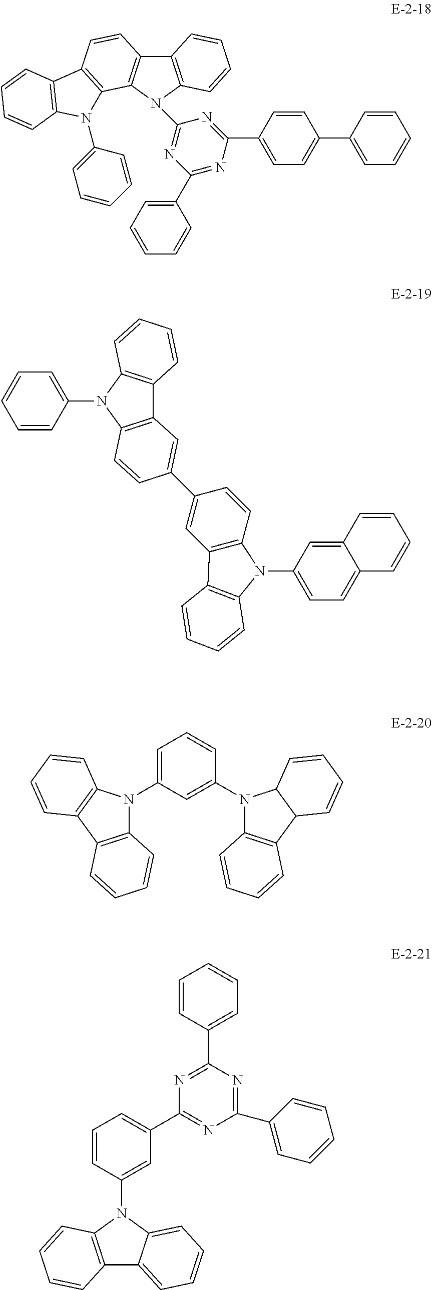 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; C1=CC2c3ccccc3N(c3cccc(-n4c5ccccc5c5ccccc54)c3)C2C=C1 ; c1ccc(-c2nc(-c3ccccc3)nc(-c3cccc(-n4c5ccccc5c5ccccc54)c3)n2)cc1]

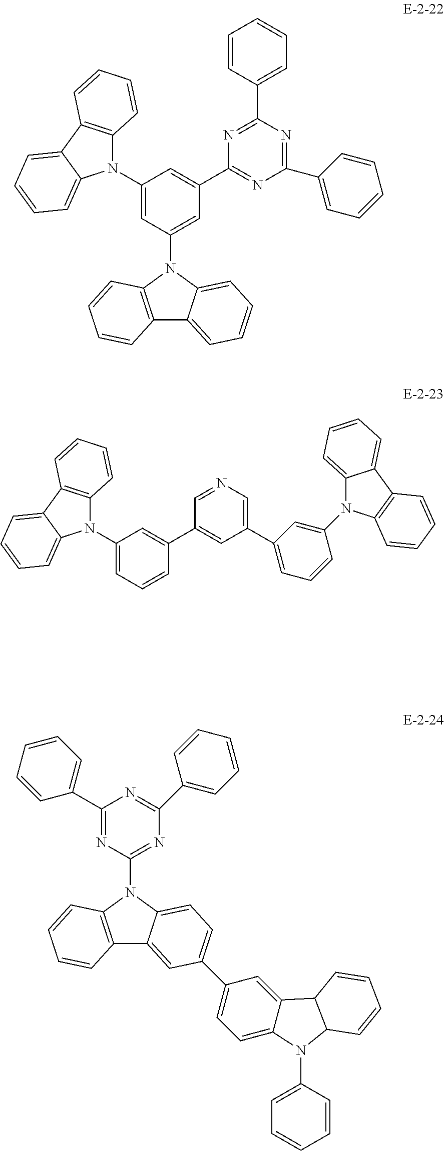 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; C1=CC2c3cc(-c4ccc5c(c4)c4ccccc4n5-c4nc(-c5ccccc5)nc(-c5ccccc5)n4)ccc3N(c3ccccc3)C2C=C1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the present disclosure is not limited thereto, for example, tris(8-hydroxyquinolino)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), etc. may also be utilized as a host material.

The emission layer EML may include a quantum dot material. The core of the quantum dot may be selected from among a Group II-VI compound, a Group III-VI compound, a Group I-III-IV compound, a Group III-V compound, a Group III-II-V compound, a Group IV-VI compound, a Group IV element, a Group IV compound, or a combination thereof.

The Group II-VI compound may be selected from among the group consisting of a binary compound selected from among the group consisting of CdSe, CdTe, CdS, ZnS, ZnSe, ZnTe, ZnO, HgS, HgSe, HgTe, MgSe, MgS, and a mixture thereof, a ternary compound selected from among the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from among the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ or In₂Se₃, a ternary compound such as InGaS₃ or InGaSes, or any combination thereof.

The Group I-III-VI compound may be selected from among a ternary compound selected from among the group consisting of AgInS, AgInS₂, CuInS, CuInS₂, AgGaS₂, CuGaS₂ CuGaO₂, AgGaO₂, AgAlO₂, and a mixture thereof, or a quaternary compound such as AgInGaS₂ or CuInGaS₂.

The Group III-V compound may be selected from among the group consisting of a binary compound selected from among the group consisting of GaN, GaP, GaAs, GaSb, AlN, AlP, AlAs, AlSb, InN, InP, InAs, InSb, and a mixture thereof, a ternary compound selected from among the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from among the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In some embodiments, the Group III-V compound may further include a Group II metal. For example, InZnP, etc., may be selected as a Group III-II-V compound.

The Group IV-VI compound may be selected from among the group consisting of a binary compound selected from among the group consisting of SnS, SnSe, SnTe, PbS, PbSe, PbTe, and a mixture thereof, a ternary compound selected from among the group consisting of SnSeS, SnSeTe, SnSTe, PbSeS, PbSeTe, PbSTe, SnPbS, SnPbSe, SnPbTe, and a mixture thereof, and a quaternary compound selected from among the group consisting of SnPbSSe, SnPbSeTe, SnPbSTe, and a mixture thereof. The Group IV element may be selected from among the group consisting of Si, Ge, and a mixture thereof. The Group IV compound may be a binary compound selected from among the group consisting of SiC, SiGe, and a mixture thereof.

Each element included in a polynary compound such as the binary compound, the ternary compound, or the quaternary compound may be present in a particle with a substantially uniform or substantially non-uniform concentration distribution. For example, the formulae refer to the types (kinds) of elements included in the compounds, and the elemental ratio in the compound may be different. For example, AgInGaS₂ may refer to AgInₓGa₁₋ₓS₂ (where x is a real number of 0 to 1).

In some embodiments, the quantum dot may have a single structure or a double structure of core-shell in which the concentration of each element included in the quantum dot is substantially uniform. For example, the material included in the core may be different from the material included in the shell.

The shell of the quantum dot may serve as a protection layer to prevent or reduce the chemical deformation of the core to maintain semiconductor properties, and/or a charging layer to impart electrophoresis properties to the quantum dot. The shell may be a single layer or multiple layers. An interface between the core and the shell may have a concentration gradient in which the concentration of an element present in the shell becomes lower towards the center.

In some embodiments, the quantum dot may have the above-described core/shell structure including a core containing nanocrystals and a shell around (e.g., surrounding) the core. An example of the shell of the quantum dots may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the embodiment of the present disclosure is not limited thereto.

Also, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

Each element included in a polynary compound such as the binary compound, or the ternary compound may be present in a particle with a substantially uniform or substantially non-uniform concentration distribution. For example, the formulae refer to the types (kinds) of elements included in the compounds, and the elemental ratio in the compound may be different.

The quantum dot may have a full width of half maximum (FWHM) of a light emitting wavelength spectrum of about 45 nm or less, about 40 nm or less, and about 30 nm or less, and color purity or color reproducibility may be improved in the above range. In some embodiments, light emitted through such quantum dot is emitted in all directions so that a wide viewing angle may be improved.

In some embodiments, although the form of the quantum dot is not particularly limited as long as it is a form commonly utilized in the art, more specifically, the quantum dot includes (e.g., in the form of) spherical, pyramidal, multi-arm, or cubic nanoparticles, nanotubes, nanowires, nanofibers, nanoplate particles, etc. may be utilized.

As the size of the quantum dot is adjusted or the elemental ratio in the quantum dot compound is adjusted, it is possible to control the energy band gap, and thus light in one or more suitable wavelength ranges may be obtained in the quantum dot emission layer. Therefore, the quantum dot as above (utilizing different sizes of quantum dots or different elemental ratios in the quantum dot compound) is utilized, and thus the light emitting device, which is configured to emit light in one or more suitable wavelengths, may be implemented. For example, the adjustment of the size of the quantum dot or the elemental ratio in the quantum dot compound may be selected to emit red, green, and/or blue light. In some embodiments, the quantum dots may be configured to emit white light by combining one or more suitable colors of light.

In each of the light emitting devices ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiment of the present disclosure is not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-2:

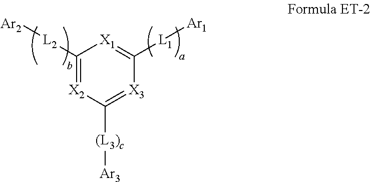

In Formula ET-2, at least one selected from among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (Balq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), or a mixture thereof.

The electron transport region ETR may include at least one selected from among Compound ET1 to Compound ET36:

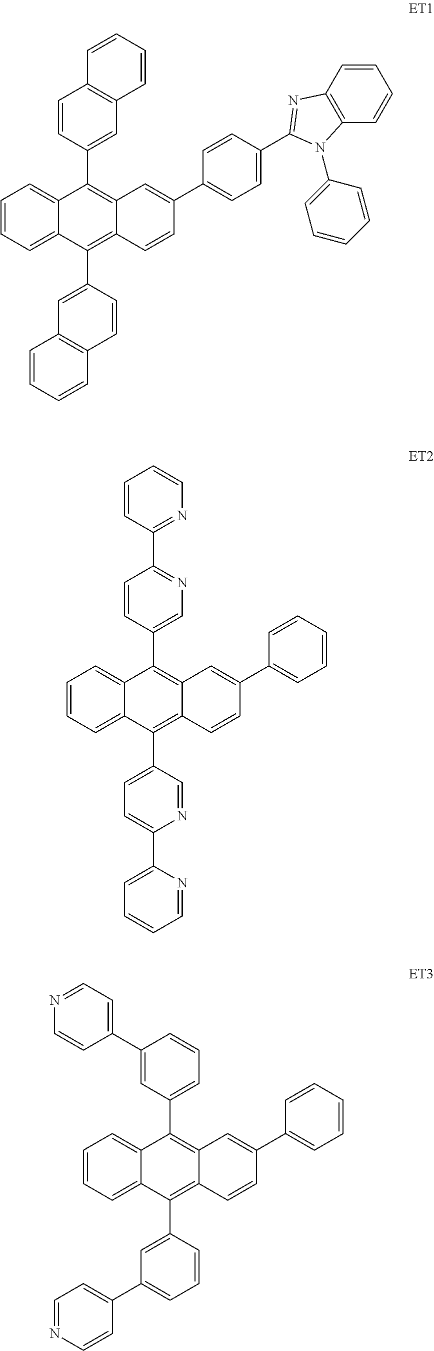 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

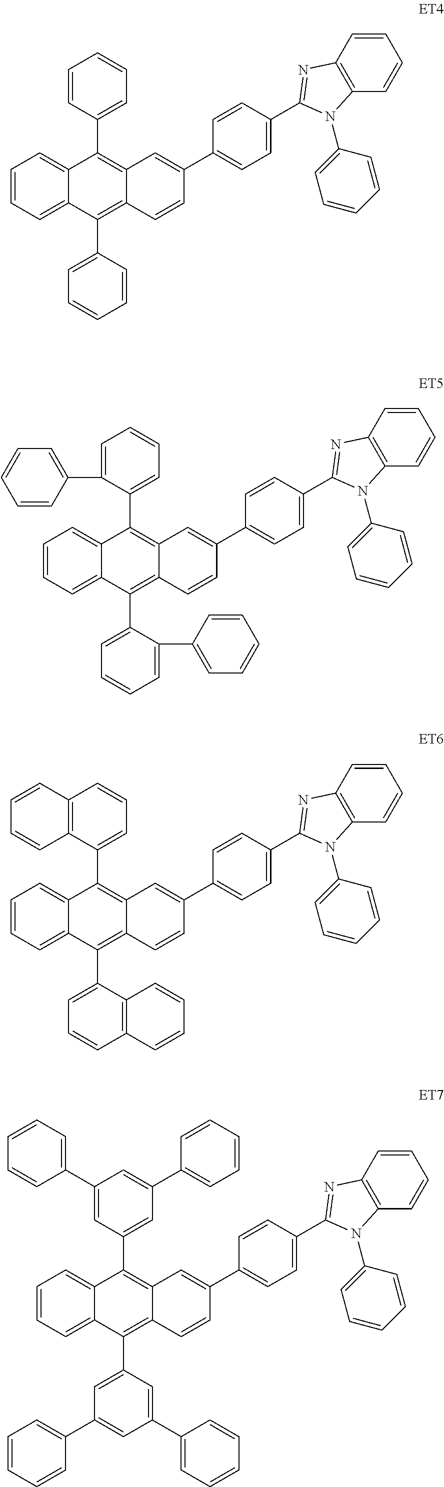 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

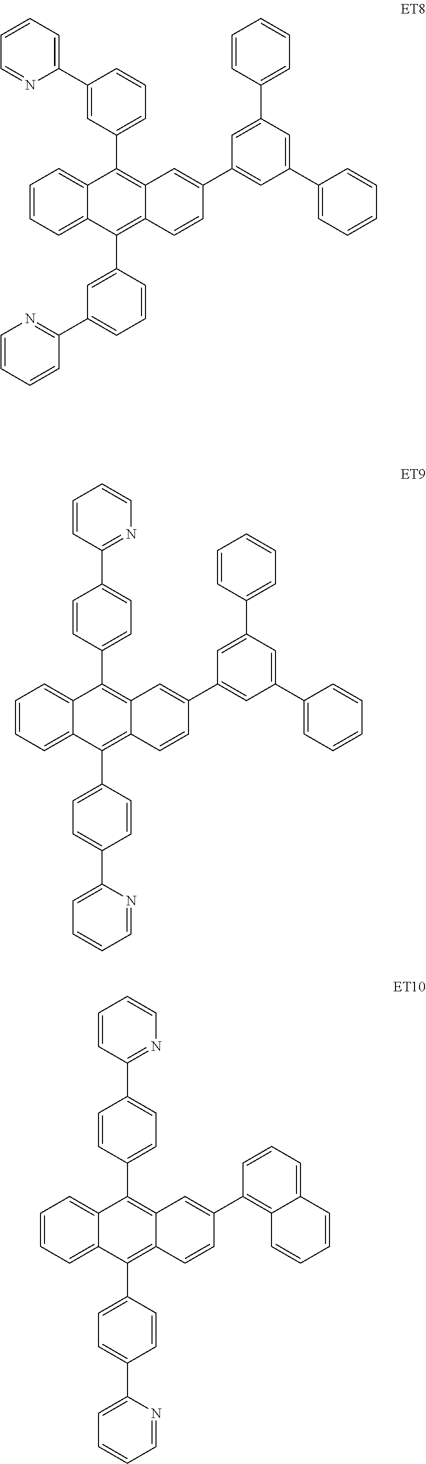 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

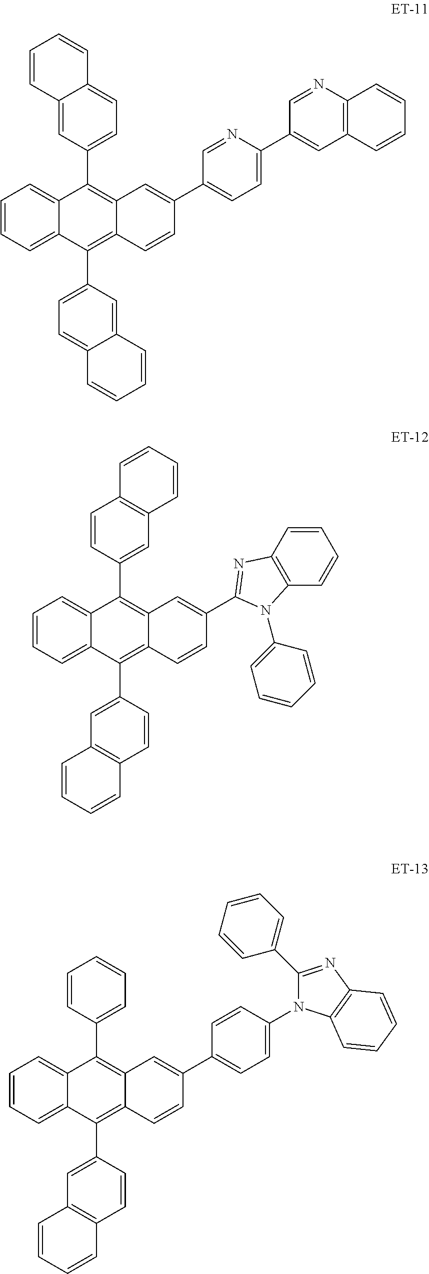 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

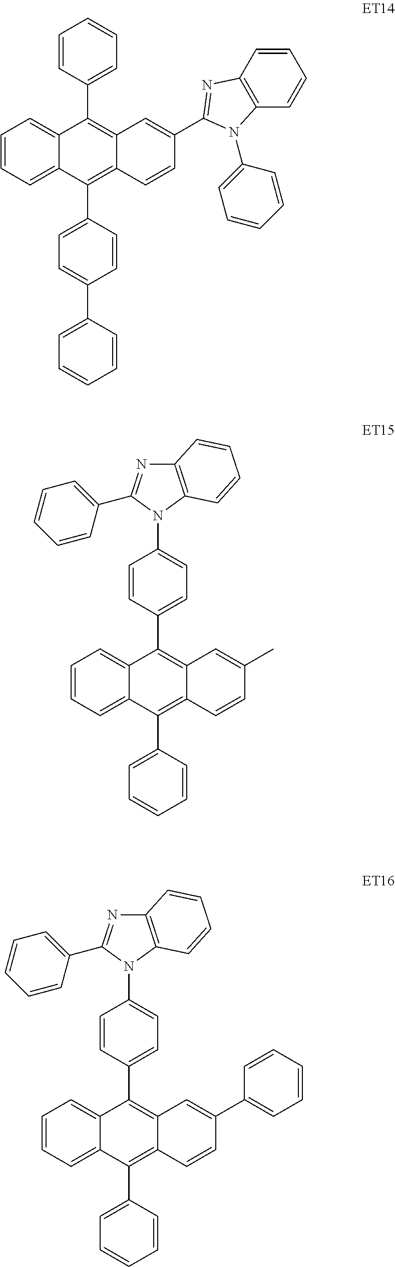 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

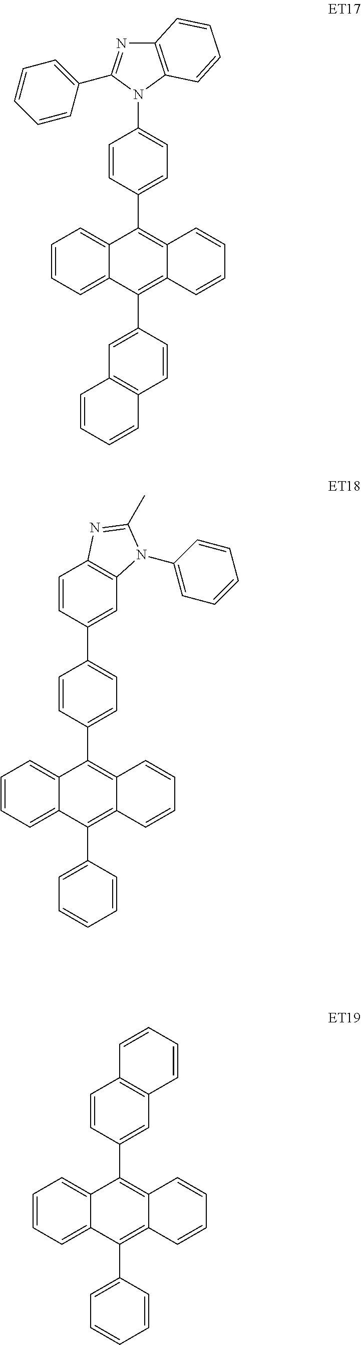

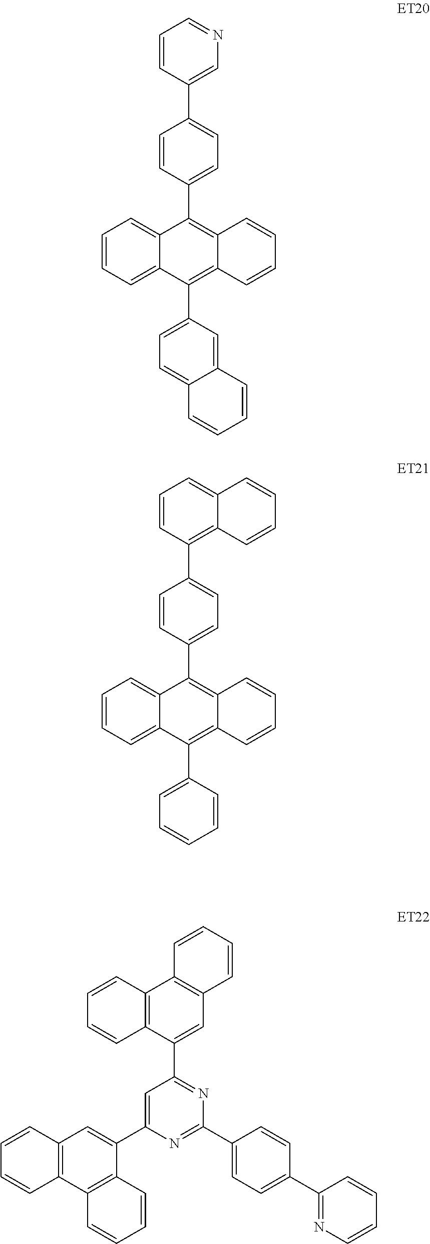

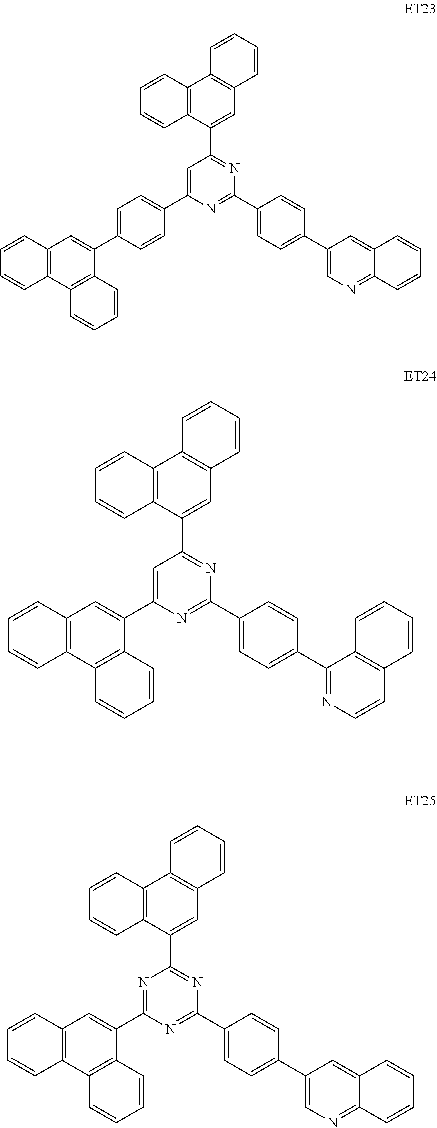 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

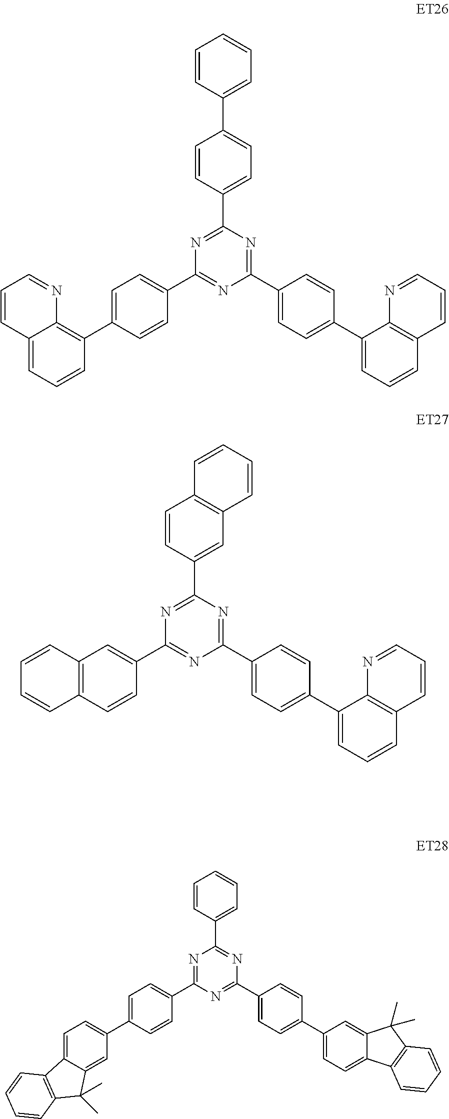 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

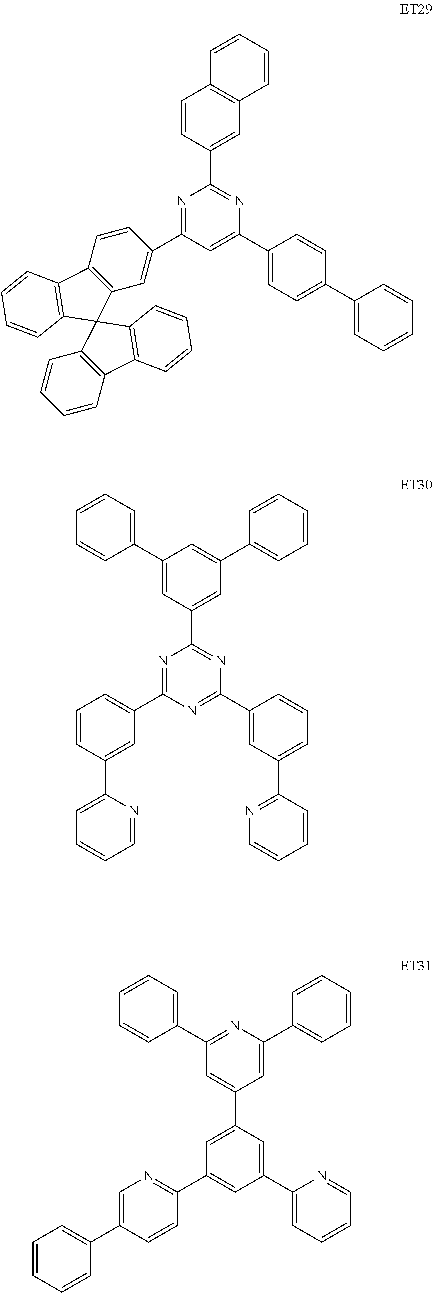 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

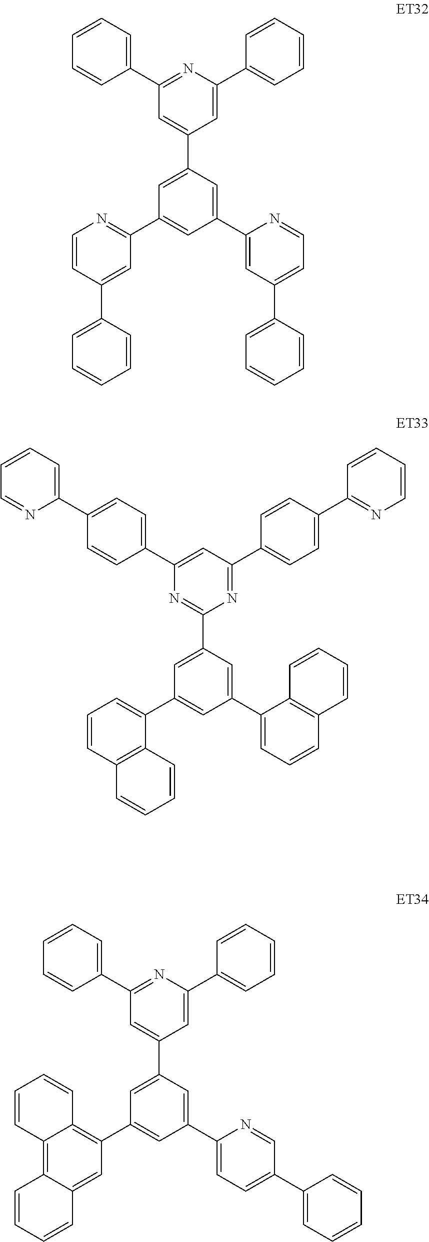 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

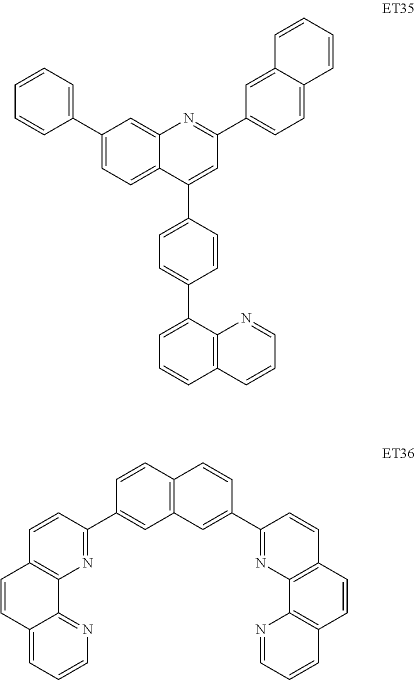 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and KI, a lanthanide metal such as Yb, and a co-deposited material of the metal halide and the lanthanide metal. For example, the electron transport region ETR may include KI:Yb, RbI:Yb, LiF:Yb, etc., as a co-deposited material. In some embodiments, the electron transport region ETR may be formed utilizing a metal oxide such as Li₂O or BaO, or 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but the embodiment of the present disclosure is not limited thereto.

The electron transport region ETR may include the above-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgYb). In some embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

In some embodiments, the second electrode EL2 may be connected with an auxiliary electrode. When the second electrode EL2 is connected with the auxiliary electrode, the resistance of the second electrode EL2 may be decreased.

In some embodiments, a capping layer CPL may further be disposed on the second electrode EL2 of the light emitting device ED of an embodiment. The capping layer CPL may include a multilayer or a single layer.

In an embodiment, the capping layer CPL may be an organic layer or an inorganic layer. For example, when the capping layer CPL contains an inorganic material, the inorganic material may include an alkaline metal compound (e.g., LiF), an alkaline earth metal compound (e.g., MgF₂), SiON, SiNₓ, SiO_{y}, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or an epoxy resin, or acrylate such as methacrylate. However, the embodiment of the present disclosure is not limited thereto, and the capping layer CPL may include at least one selected from among Compounds P1 to P5:

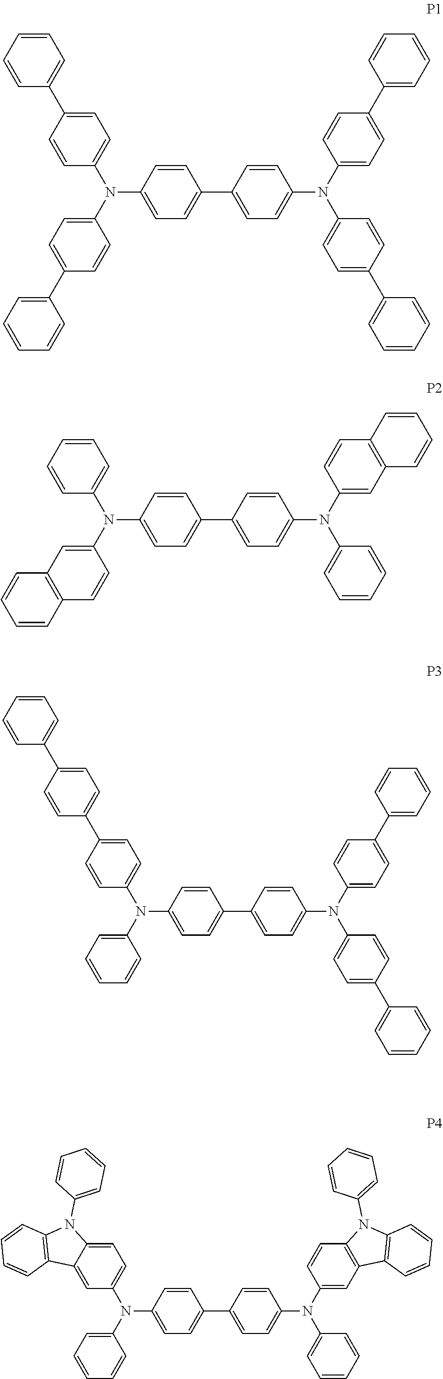 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

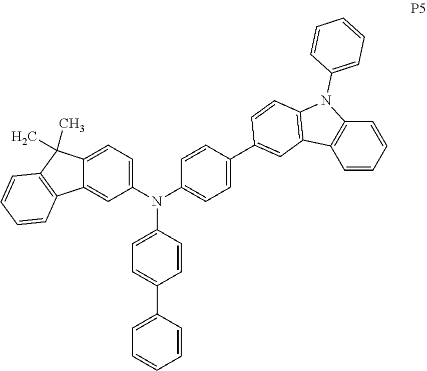 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

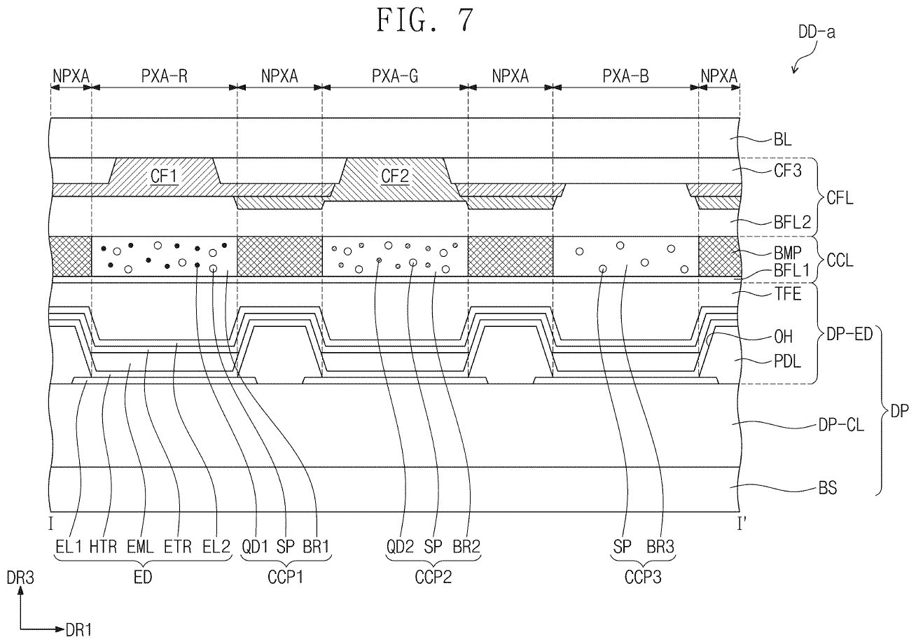

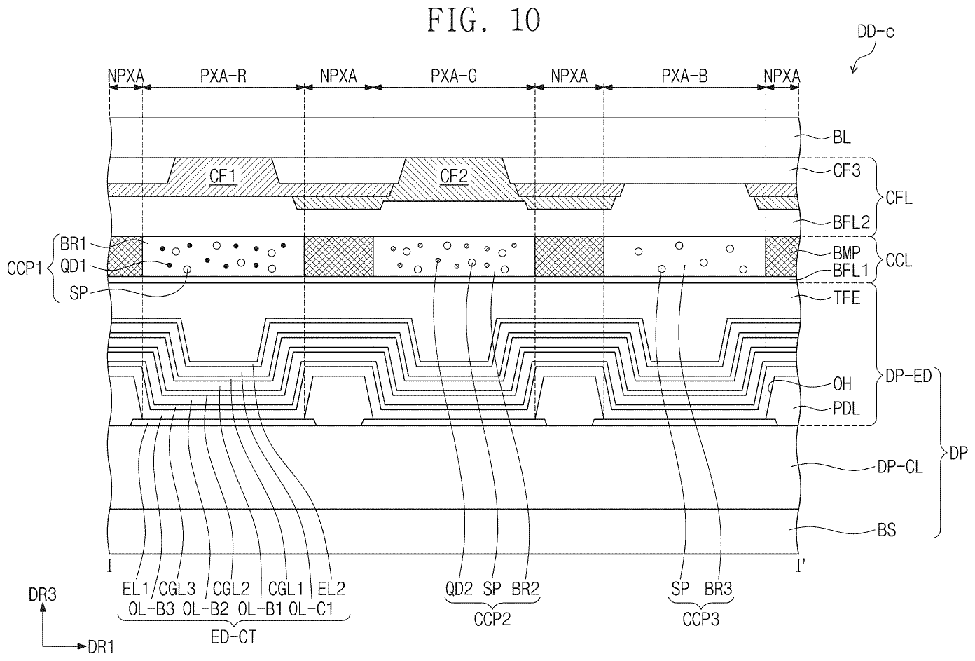

Each of FIGS. 7 to 10 is a cross-sectional view of a display apparatus according to an embodiment of the present disclosure. Hereinafter, in describing the display apparatuses of embodiments with reference to FIGS. 7 to 10, the duplicated features which have been described in FIGS. 1 to 6 are not described again, but their differences will be mainly described.

Referring to FIG. 7, the display apparatus DD-a according to an embodiment may include a display panel DP including a display device layer DP-ED, a light control layer CCL disposed on the display panel DP, and a color filter layer CFL. In an embodiment illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In some embodiments, the structures of the light emitting devices of FIGS. 3 to 6 as described above may be equally applied to the structure of the light emitting device ED illustrated in FIG. 7.

The hole transport region HTR of the light-emitting element ED included in a display device DD according to an embodiment may include the above-described amine compound according to an embodiment.

Referring to FIG. 7, the emission layer EML may be disposed in an opening OH defined in a pixel defining film PDL. For example, the emission layer EML which is divided by the pixel defining film PDL and provided corresponding to each light emitting regions PXA-R, PXA-G, and PXA-B may be configured to emit light in substantially the same wavelength range. In the display apparatus DD-a of an embodiment, the emission layer EML may be configured to emit blue light. In some embodiments, unlike the configuration illustrated, in an embodiment, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, and/or the like. The light conversion body may be configured to emit provided light by converting the wavelength thereof. For example, the light control layer CCL may include a layer containing the quantum dot or a layer containing the phosphor.

The light control layer CCL may include a plurality of light control parts CCP1, CCP2 and CCP3. The light control parts CCP1, CCP2, and CCP3 may be spaced apart from each other.

Referring to FIG. 7, divided patterns BMP may be disposed between the light control parts CCP1, CCP2 and CCP3 which are spaced apart from each other, but the embodiment of the present disclosure is not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting device ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting device ED. For example, the first quantum dot QD1 may be a red quantum dot, and the second quantum dot QD2 may be a green quantum dot. The same as described above may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2 and CCP3 from being exposed to moisture/oxygen. In some embodiments, the barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display apparatus DD-a of an embodiment, the color filter layer CFL may be disposed on the light control layer CCL. For example, the color filter layer CFL may be directly disposed on the light control layer CCL. In this case, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye.

In some embodiments, the embodiment of the present disclosure is not limited thereto, and the third filter CF3 may not include (e.g., may exclude) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter.

In some embodiments, the color filter layer CFL may further include a light shielding part. The light shielding part may be a black matrix. The light shielding part may include an organic light shielding material or an inorganic light shielding material containing a black pigment or dye. The light shielding part may prevent or reduce light leakage, and may separate boundaries between the adjacent filters CF1, CF2, and CF3.

The first to third filters CF1, CF2, and CF3 may be disposed corresponding to the red light emitting region PXA-R, the green light emitting region PXA-G, and the blue light emitting region PXA-B, respectively.

A base substrate BL may be disposed on the color filter layer CFL. The base substrate BL may be a member which provides a base surface in which the color filter layer CFL, the light control layer CCL, and/or the like are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, unlike the configuration illustrated, in an embodiment, the base substrate BL may not be provided.

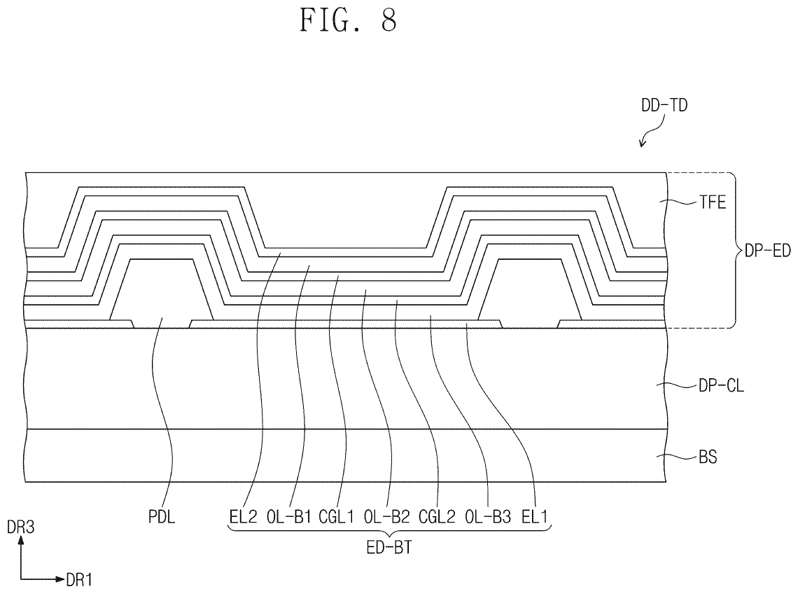

FIG. 8 is a cross-sectional view illustrating a portion of a display apparatus according to an embodiment. In the display apparatus DD-TD of an embodiment, the light emitting device ED-BT may include a plurality of light emitting structures OL-B1, OL-B2, and OL-B3. The light emitting device ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) located therebetween.

For example, the light emitting device ED-BT included in the display apparatus DD-TD of an embodiment may be a light emitting device having a tandem structure and including a plurality of emission layers.

In an embodiment illustrated in FIG. 8, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting device ED-BT including the plurality of light emitting structures OL-B1, OL-B2, and OL-B3 which are configured to emit light beams having wavelength ranges different from each other may be configured to emit white light.

Charge generation layers CGL1 and CGL2 may be respectively disposed between two of the neighboring light emitting structures OL-B1, OL-B2, and OL-B3. The charge generation layers CGL1 and CGL2 may include a p-type or kind charge generation layer and/or an n-type or kind charge generation layer.

At least one selected from among the light-emitting structures OL-B1, OL-B2, and OL-B3 included in the display device DD-TD according to an embodiment may include the above-described amine compound according to an embodiment. For example, at least one selected from among a plurality of the hole transport regions included in a light-emitting element ED-BT may contain the amine compound according to an embodiment.

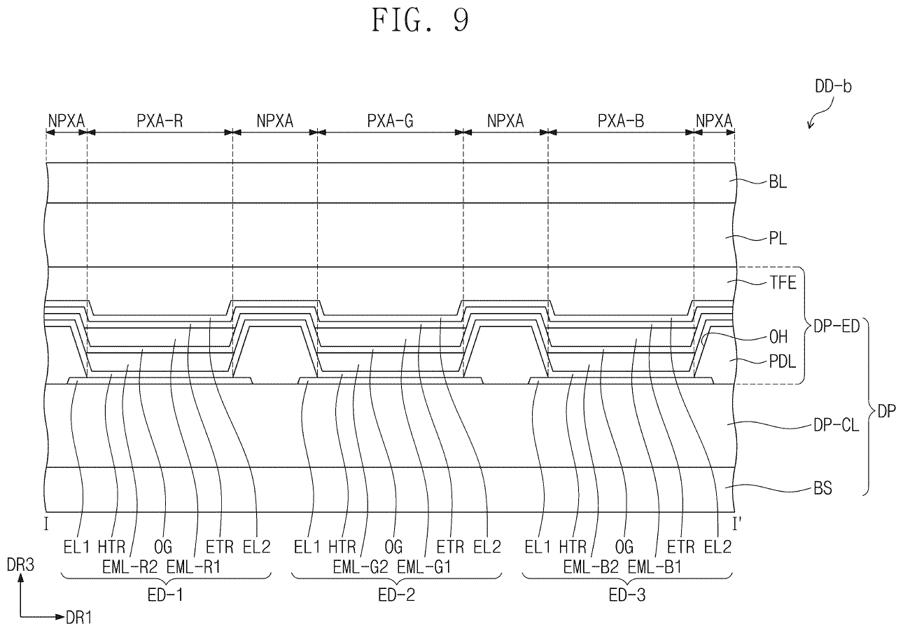

Referring to FIG. 9, the display apparatus DD-b according to an embodiment may include light emitting devices ED-1, ED-2, and ED-3 in which two emission layers are stacked. Compared with the display apparatus DD of an embodiment illustrated in FIG. 2, an embodiment illustrated in FIG. 9 has a difference in that the first to third light emitting devices ED-1, ED-2, and ED-3 each include two emission layers stacked in the thickness direction. In each of the first to third light emitting devices ED-1, ED-2, and ED-3, the two emission layers may be configured to emit light in substantially the same wavelength region.

The first light emitting device ED-1 may include a first red emission layer EML-R1 and a second red emission layer EML-R2. The second light emitting device ED-2 may include a first green emission layer EML-G1 and a second green emission layer EML-G2. In some embodiments, the third light emitting device ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. More specifically, the emission auxiliary part OG may include an electron transport region, a charge generation layer, and a hole transport region that are sequentially stacked. The emission auxiliary part OG may be provided as a common layer in the whole of the first to third light emitting devices ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be disposed between the emission auxiliary part OG and the electron transport region ETR. The second red emission layer EML-R2, the second green emission layer EML-G2, and the second blue emission layer EML-B2 may be disposed between the hole transport region HTR and the emission auxiliary part OG.

For example, the first light emitting device ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting device ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting device ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In some embodiments, an optical auxiliary layer PL may be disposed on the display device layer DP-ED. The optical auxiliary layer PL may include a polarizing layer. The optical auxiliary layer PL may be disposed on the display panel DP and control reflected light in the display panel DP due to external light. Unlike the configuration illustrated, the optical auxiliary layer PL in the display apparatus according to an embodiment may not be provided.

At least one hole transport region HTR included in the display device DD-b according to an embodiment, illustrated in FIG. 9, may contain the above-described amine compound according to an embodiment. For example, in an embodiment, at least one selected from among the hole transport regions HTRs included in light-emitting elements ED-1, ED-2, and ED-3 may contain the amine compound according to an embodiment.

Unlike FIGS. 8 and 9, FIG. 10 illustrates that a display apparatus DD-c includes four light emitting structures OL-B1, OL-B2, OL-B3, and OL-C1. A light emitting device ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be disposed between the first to fourth light emitting structures OL-B1, OL-B2, OL-B3, and OL-C1. Among the four light emitting structures, the first to third light emitting structures OL-B1, OL-B2, and OL-B3 may be configured to emit blue light, and the fourth light emitting structure OL-C1 may be configured to emit green light. However, the embodiment of the present disclosure is not limited thereto, and the first to fourth light emitting structures OL-B1, OL-B2, OL-B3, and OL-C1 may be configured to emit light beams in different wavelength regions.

The charge generation layers CGL1, CGL2, and CGL3 disposed between adjacent light emitting structures OL-B1, OL-B2, OL-B3, and OL-C1 may include a p-type or kind charge generation layer and/or an n-type or kind charge generation layer.

At least one of the light-emitting structures OL-B1, OL-B2, OL-B3, or OL-C1 included in the display device DD-c according to an embodiment may contain the above-described amine compound according to an embodiment.

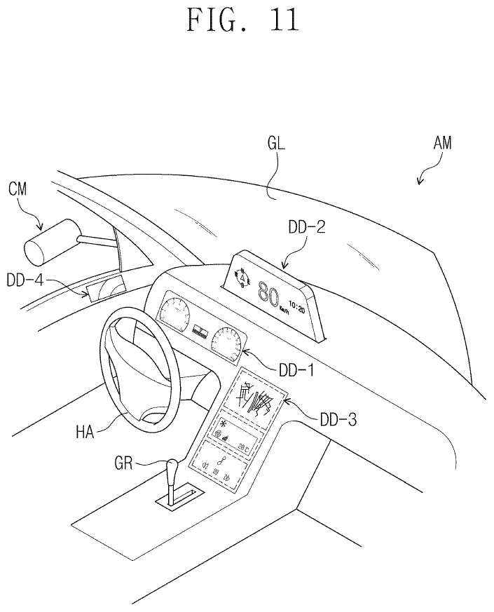

FIG. 11 is a view illustrating a vehicle in which the display device according to an embodiment is disposed.

In an embodiment, the electronic apparatus may include a display apparatus including a plurality of light emitting devices, and a control part which controls the display apparatus. The electronic apparatus of an embodiment may be a device that is activated according to an electrical signal. The electronic apparatus may include display apparatuses of one or more suitable embodiments. For example, the electronic apparatus may include not only large-sized electronic apparatuses such as a television set, a monitor, or an outdoor billboard but also include small- and medium-sized electronic apparatuses such as a personal computer, a laptop computer, a personal digital terminal, a display apparatus for a vehicle, a game console, a portable electronic device, or a camera.

FIG. 11 is a view illustrating a vehicle AM in which first to fourth display apparatuses DD-1, DD-2, DD-3, and DD-4 are disposed. At least one selected from among the first to fourth display apparatuses DD-1, DD-2, DD-3, and DD-4 may include the same configuration as the display apparatuses DD, DD-TD, DD-a, DD-b, and DD-c as described with reference to FIGS. 1, and 2, and 7 to 10.

FIG. 11 illustrates a vehicle AM, but this is an example, and the first to fourth display apparatuses DD-1, DD-2, DD-3, and DD-4 may be disposed in another transportation refers to such as bicycles, motorcycles, trains, ships, and airplanes. In some embodiments, at least one selected from among the first to fourth display apparatuses DD-1, DD-2, DD-3, and DD-4 including the same configuration as the display apparatuses DD, DD-TD, DD-a, DD-b, and DD-c of an embodiment may be employed in a personal computer, a laptop computer, a personal digital terminal, a game console, a portable electronic device, a television, a monitor, an outdoor billboard, and/or the like. In some embodiments, these are merely provided as embodiments, and thus may be employed in other electronic apparatuses unless departing from the present disclosure.

At least one selected from among the first to fourth display apparatuses DD-1, DD-2, DD-3, and DD-4 may include the light emitting device ED of an embodiment as described with reference to FIGS. 3 to 6.

The light emitting element ED according to an embodiment may contain the amine compound according to an embodiment. At least one selected from among the first to fourth display devices DD-1, DD-2, DD-3, and DD-4 includes the light-emitting element ED containing the amine compound according to an embodiment, and thus display lifespan may be improved.

Referring to FIG. 11, the vehicle AM may include a steering wheel HA and a gear GR for driving the vehicle AM. In some embodiments, the vehicle AM may include a front window GL disposed so as to face the driver.

The first display apparatus DD-1 may be disposed in a first region overlapping the steering wheel HA. For example, the first display apparatus DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, etc. A first scale and a second scale may be indicated as a digital image.

The second display apparatus DD-2 may be disposed in a second region facing the driver's seat and overlapping the front window GL. The driver's seat may be a seat in which the steering wheel HA is disposed. For example, the second display apparatus DD-2 may be a head up display (HUD) which displays second information of the vehicle AM. The second display apparatus DD-2 may be optically transparent. The second information may include digital numbers which indicate a driving speed, and may further include information such as the current time. Unlike the configuration illustrated, the second information of the second display apparatus DD-2 may be projected to the front window GL to be displayed.

The third display apparatus DD-3 may be disposed in a third region adjacent to the gear GR. For example, the third display apparatus DD-3 may be disposed between the driver's seat and the passenger seat and may be a center information display (CID) for a vehicle for displaying third information. The passenger seat may be a seat spaced apart from the driver's seat with the gear GR disposed therebetween. The third information may include information about traffic (e.g., navigation information), playing music or radio or a video (or an image), temperatures inside the vehicle AM, etc.

The fourth display apparatus DD-4 may be spaced apart from the steering wheel HA and the gear GR, and may be disposed in a fourth region adjacent to the side of the vehicle AM. For example, the fourth display apparatus DD-4 may be a digital side-view mirror which displays fourth information. The fourth display apparatus DD-4 may display an image outside the vehicle AM taken by a camera module CM disposed outside the vehicle AM. The fourth information may include an image outside the vehicle AM.

The above-described first to fourth information may be examples, and the first to fourth display apparatuses DD-1, DD-2, DD-3, and DD-4 may further display information about the inside and outside of the vehicle AM. The first to fourth information may include different information. However, the embodiment of the present disclosure is not limited thereto, and a part of the first to fourth information may include the same information as one another.

Hereinafter, with reference to Examples and Comparative Examples, a compound according to an embodiment of the present disclosure and a light-emitting element according to an embodiment will be described in more detail. In some embodiments, Examples described are only illustrations to assist in understanding the present disclosure, but the scope of the present disclosure is not limited thereto.

### EXAMPLES

### 1. Synthesis of Compound 1

Amine compound 1 according to one Example may be synthesized by, for example, Reaction Scheme 1.

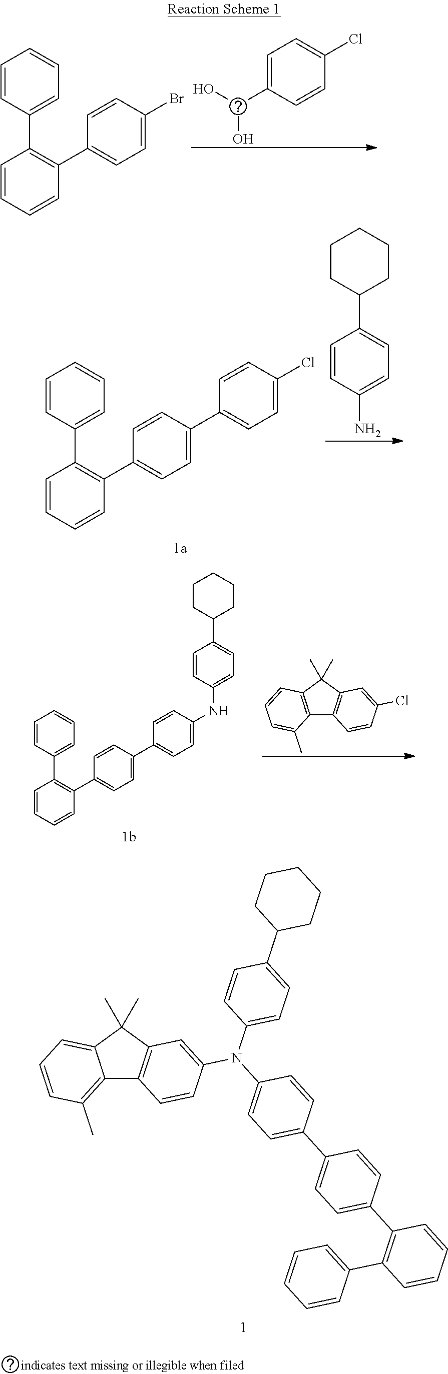

### Synthesis of Intermediate 1a

(4-bromophenyl)boronic acid (1.1 eq.), 4-bromo-1,1′:2′,1″-terphenyl (1.0 eq.), tetrakis(triphenylphosphine)palladium (0.1 eq.), and potassium carbonate (3.0 eq.) were dissolved in about 200 mL of toluene, about 40 mL of ethanol, and about 80 mL of water, and then, in a nitrogen atmosphere, the resultant mixture was stirred at about 110° C. for about 24 hours. When the reaction was completed, the resultant product was washed three times with water and diethyl ether. Then, the obtained organic layer was dried over MgSO₄, and then dried under reduced pressure. Intermediate 1a was obtained by column chromatography. (yield: about 75%)

### Synthesis of Intermediate 1b

Intermediate 1a (1.0 eq.), 4-cyclohexylaniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 1 b was obtained by column chromatography. (yield: about 68%)

### Synthesis of Compound 1

Intermediate 1 b (1.0 eq.), 2-chloro-5,9,9-trimethyl-9H-fluorene (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 1 was obtained by column chromatography. (yield: about 70%)

### 2. Synthesis of Compound 6

Amine Compound 6 according to one Example may be synthesized by, for example, Reaction Scheme 2.

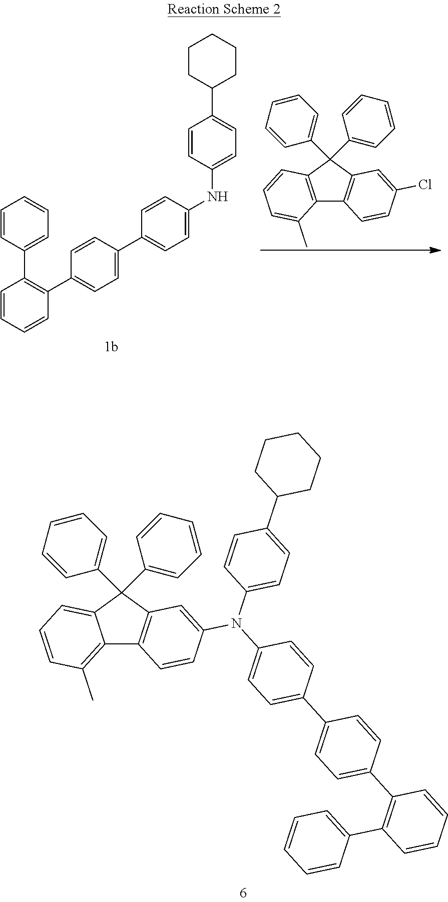

### Synthesis of Compound 6

Intermediate 1 b (1.0 eq.), 2-chloro-5-methyl-9,9-diphenyl-9H-fluorene (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 6 was obtained by column chromatography. (yield: about 73%)

### 3. Synthesis of Compound 27

Amine Compound 27 according to an embodiment may be synthesized by, for example, Reaction Scheme 3.

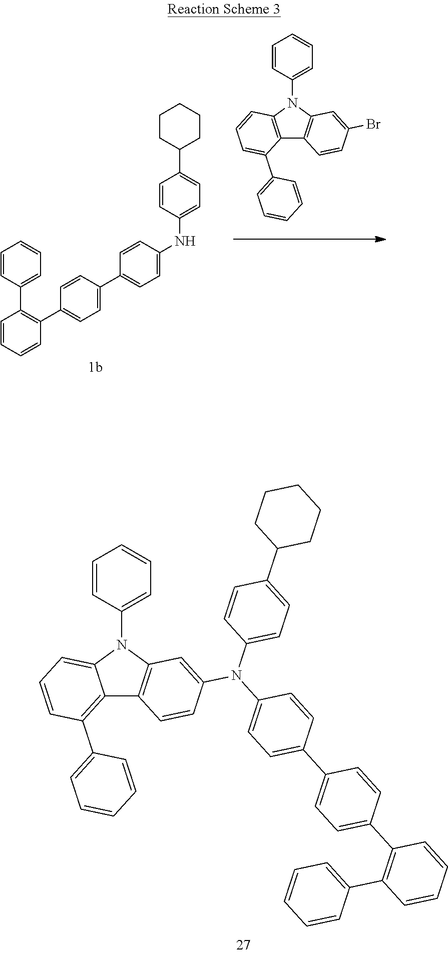

### Synthesis of Compound 27

Intermediate 1 b (1.0 eq.), 2-bromo-5,9-diphenyl-9H-carbazole (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 ML of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 27 was obtained by column chromatography. (yield: about 69%) 4. Synthesis of Compound 42

Amine Compound 42 according to one Example may be synthesized by, for example, Reaction Scheme 4.

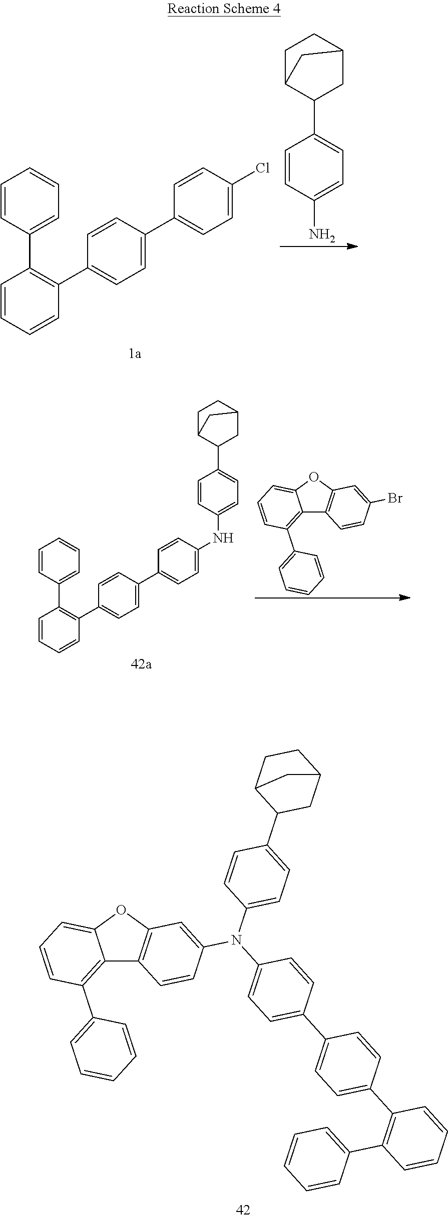

### Synthesis of Intermediate 42a

Intermediate 1a (1.0 eq.), 4-(bicyclo[2.2.1]heptan-2-yl)aniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 42a was obtained by column chromatography. (yield: about 65%)

### Synthesis of Compound 42

Intermediate 42a (1.0 eq.), 7-bromo-1-phenyldibenzo[b,d]furan (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 42 was obtained by column chromatography. (yield: about 72%)

### 5. Synthesis of Compound 63

Amine Compound 63 according to one Example may be synthesized by, for example, Reaction Scheme 5.

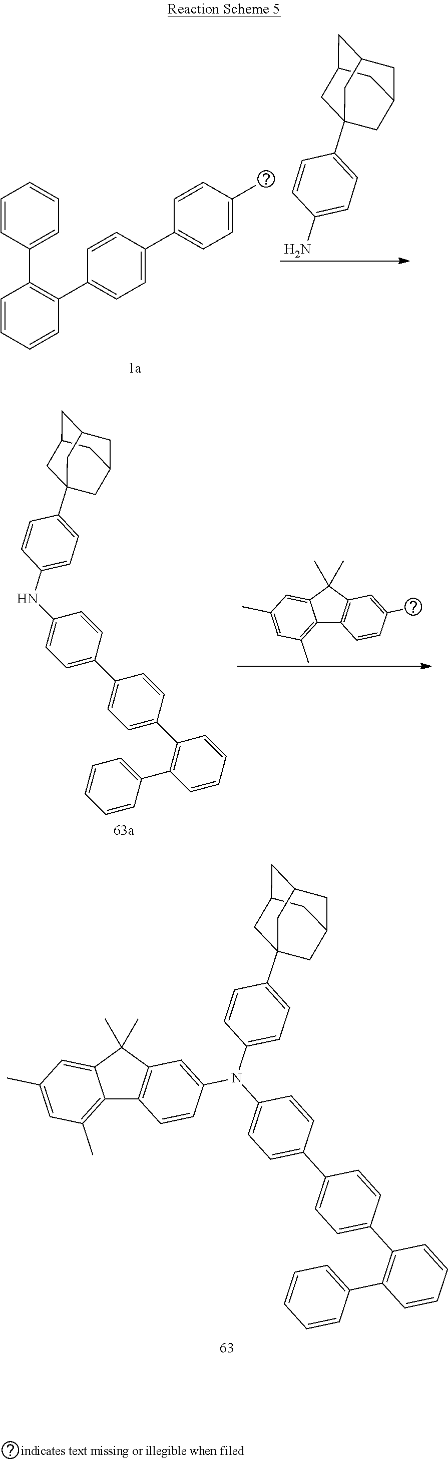

### Synthesis of Intermediate 63a

Intermediate 1a ((1.0 eq.), 4-((1R,3s)-adamantan-1-yl)aniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 63a was obtained by column chromatography. (yield: about 63%)

### Synthesis of Compound 63

Intermediate 63a (1.0 eq.), 7-bromo-2,4,9,9-tetramethyl-9H-fluorene-1-methane (1/1) (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 63 was obtained by column chromatography. (yield: about 70%)

### 6. Synthesis of Compound 96

Amine Compound 96 according to one Example may be synthesized by, for example, Reaction Scheme 6.

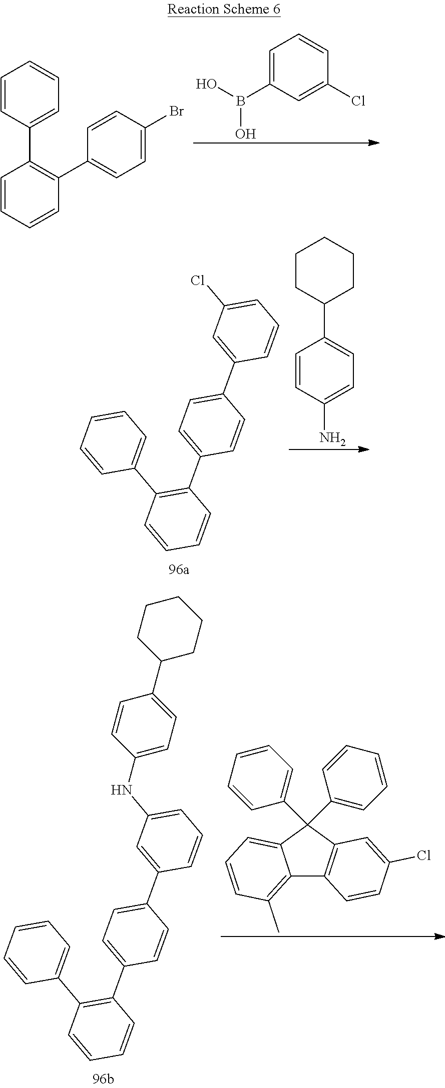

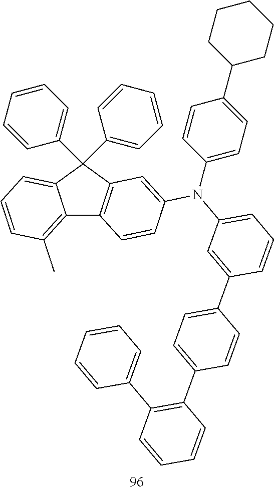 [SMILES: Cc1cccc2c1-c1ccc(N(c3ccc(C4CCCCC4)cc3)c3cccc(-c4ccc(-c5ccccc5-c5ccccc5)cc4)c3)cc1C2(c1ccccc1)c1ccccc1]

### Synthesis of Intermediate 96a

(3-chlorophenyl)boronic acid (1.1 eq.), 4-bromo-1,1′:2′,1″-terphenyl (1.0 eq.), tetrakis(triphenylphosphine)palladium (0.1 eq.), and potassium carbonate (3.0 eq.) were dissolved in about 200 mL of toluene, about 40 mL of ethanol, and about 80 mL of water, and then the resultant mixture was stirred in a nitrogen atmosphere at about 110° C. for about 24 hours. When the reaction was completed, the resultant product was washed three times with water and diethyl ether. Then, the obtained organic layer was dried over MgSO₄, and then dried under reduced pressure. Intermediate 96a was obtained by column chromatography. (yield: about 76%)

### Synthesis of Intermediate 96b

Intermediate 96a (1.0 eq.), 4-cyclohexylaniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 96b was obtained by column chromatography. (yield: about 70%)

### Synthesis of Compound 96

Intermediate 96b (1.0 eq.), 2-chloro-5-methyl-9,9-diphenyl-9H-fluorene (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 96 was obtained by column chromatography. (yield: about 67%)

### 7. Synthesis of Compound 137

Amine Compound 137 according to one Example may be synthesized by, for example, Reaction Scheme 7.

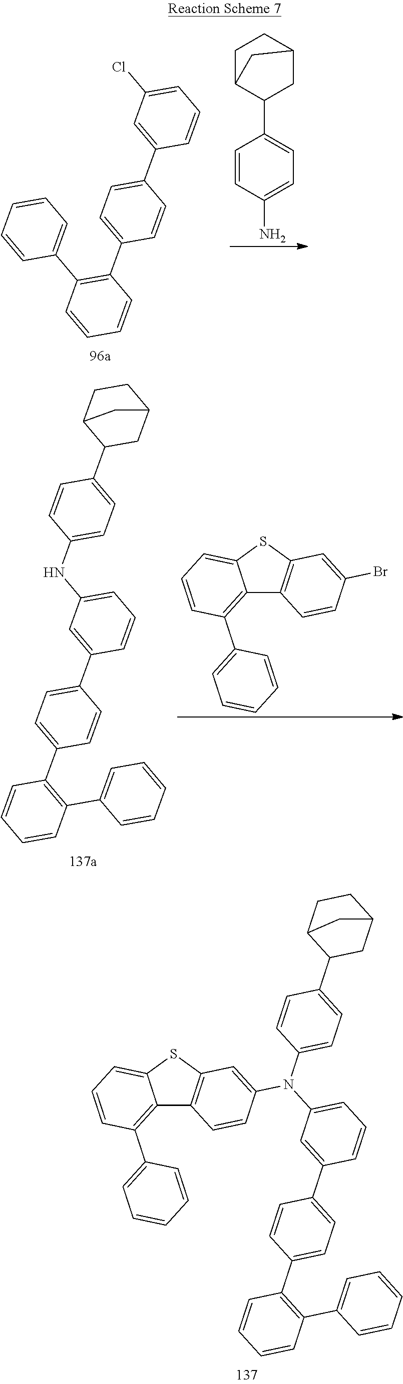

### Synthesis of Intermediate 137a

Intermediate 96a (1.0 eq.), 4-(bicyclo[2.2.1]heptan-2-yl)aniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 137a was obtained by column chromatography. (yield: about 61%)

### Synthesis of Compound 137

Intermediate 137a (1.0 eq.), 7-bromo-1-phenyldibenzo[b,d]thiophene (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 137 was obtained by column chromatography. (yield: about 73%)

### 8. Synthesis of Compound 151

Amine Compound 151 according to one Example may be synthesized by, for example, Reaction Scheme 8.

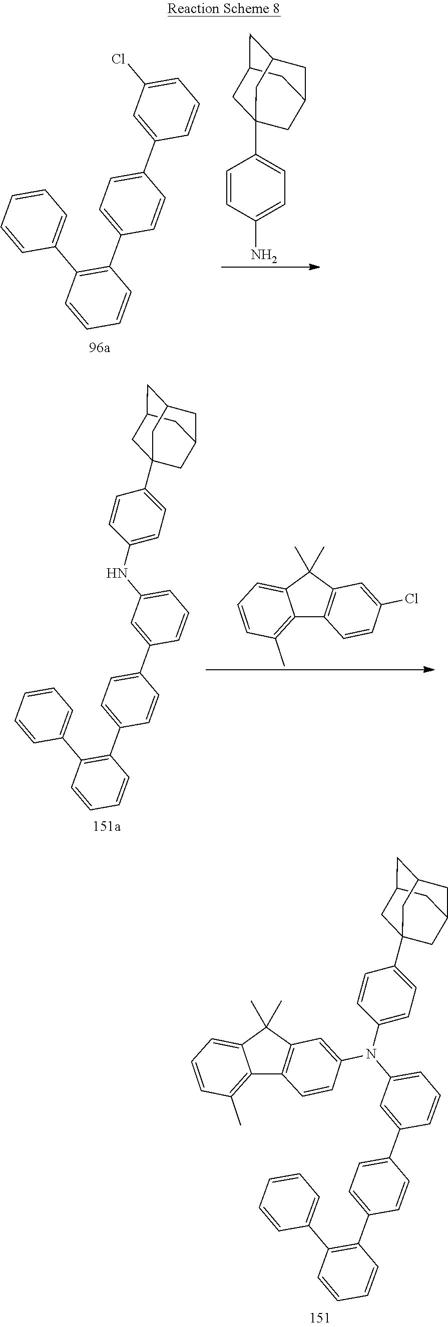

### Synthesis of Intermediate 151a

Intermediate 96a (1.0 eq.), 4-((3r,5r,7r)-adamantan-1-yl)aniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 151a was obtained by column chromatography. (yield: about 64%)

### Synthesis of Compound 151

Intermediate 151a (1.0 eq.), 2-chloro-5,9,9-trimethyl-9H-fluorene (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 151 was obtained by column chromatography. (yield: about 70%)

### 9. Synthesis of Compound 193

Amine Compound 193 according to one Example may be synthesized by, for example, Reaction Scheme 9.

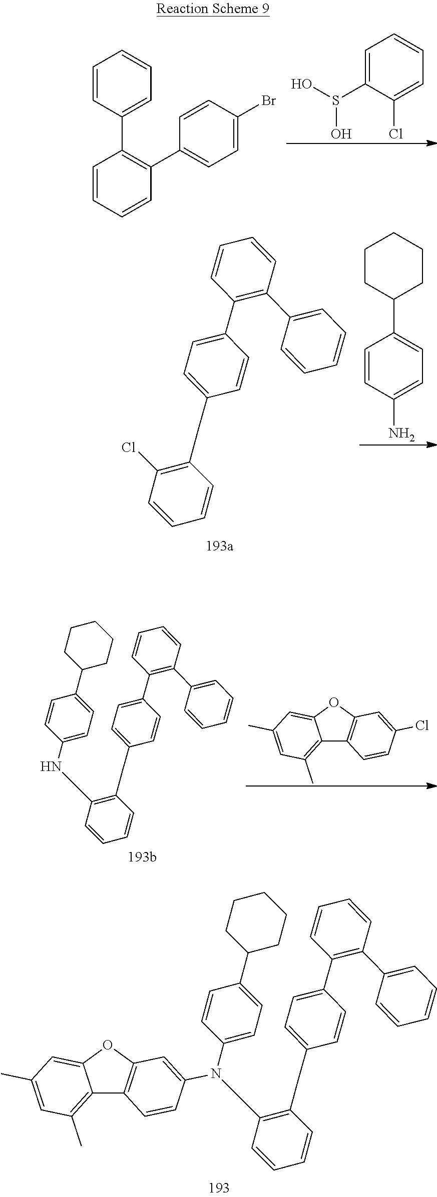

### Synthesis of Intermediate 193a

(2-chlorophenyl)boronic acid (1.1 eq.), 4-bromo-1,1′:2′,1″-terphenyl (1.0 eq.), tetrakis(triphenylphosphine)palladium (0.1 eq.), and potassium carbonate (3.0 eq.) were dissolved in about 200 mL of toluene, about 40 mL of ethanol, and about 80 mL of water, and then the resultant mixture was stirred in a nitrogen atmosphere at about 110° C. for about 24 hours. When the reaction was completed, the resultant product was washed three times with water and diethyl ether. Then, the obtained organic layer was dried over MgSO₄, and then dried under reduced pressure. Intermediate 193a was obtained by column chromatography. (yield: about 72%)

### Synthesis of Intermediate 193b

Intermediate 193a (1.0 eq.), 4-cyclohexylaniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 193b was obtained by column chromatography. (yield: about 65%)

### Synthesis of Compound 193

Intermediate 193b (1.0 eq.), (1.0 eq.), 7-chloro-1,3-dimethyldibenzo[b,d]furan (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 193 was obtained by column chromatography. (yield: about 60%)

### 10. Synthesis of Compound 217

Amine Compound 217 according to one Example may be synthesized by, for example, Reaction Scheme 10.

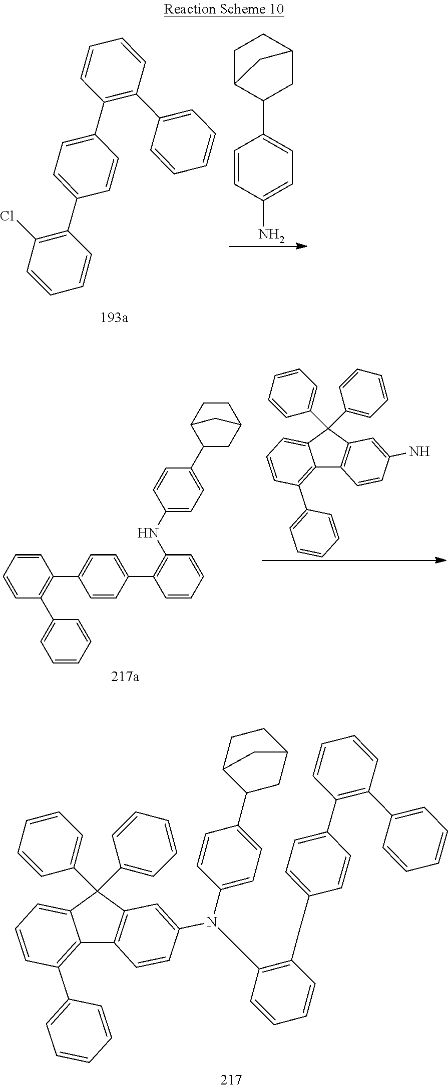

### Synthesis of Intermediate 217a

Intermediate 193a (1.0 eq.), 4-(bicyclo[2.2.1]heptan-2-yl)aniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 217a was obtained by column chromatography. (yield: about 63%)

### Synthesis of Compound 217

Intermediate 217a (1.0 eq.), 2-bromo-5,9,9-triphenyl-9H-fluorene (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 217 was obtained by column chromatography. (yield: about 65%)

### 11. Synthesis of Compound 271

Amine Compound 271 according to one Example may be synthesized by, for example, Reaction Scheme 11.

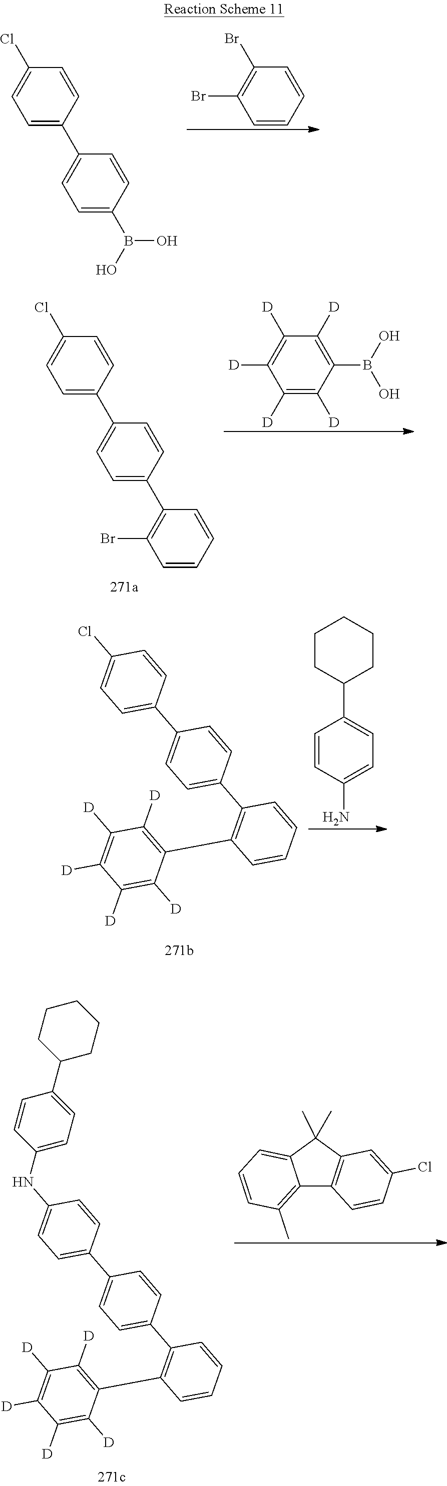

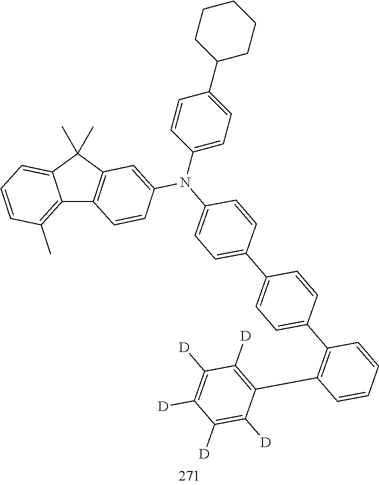 [SMILES: [2H]c1c([2H])c([2H])c(Cc2ccccc2-c2ccc(-c3ccc(N(c4ccc(C5CCCCC5)cc4)c4ccc5c(c4)C(C)(C)c4cccc(C)c4-5)cc3)cc2)c([2H])c1[2H]]

### Synthesis of Intermediate 271a

(4′-chloro-[1,1′-biphenyl]-4-yl)boronic acid (1.1 eq.), 1,2-dibromobenzene (1.0 eq.), tetrakis(triphenylphosphine)palladium (0.1 eq.), and potassium carbonate (3.0 eq.) were dissolved in about 200 mL of toluene, about 40 mL of ethanol, and about 80 mL of water, and then the resultant mixture was stirred in a nitrogen atmosphere at about 110° C. for about 24 hours. When the reaction was completed, the resultant product was washed three times with water and diethyl ether. Then, the obtained organic layer was dried over MgSO₄, and then dried under reduced pressure. Intermediate 271a was obtained by column chromatography. (yield: about 60%)

### Synthesis of Intermediate 271b

(phenyl-d5)boronic acid (1.0 eq.), Intermediate 271a (1.1 eq.), tetrakis(triphenylphosphine)palladium (0.1 eq.), and potassium carbonate (3.0 eq.) were dissolved in about 200 mL of toluene, about 40 mL of ethanol, and about 80 mL of water and then the resultant mixture was stirred in a nitrogen atmosphere at about 110° C. for about 24 hours. When the reaction was completed, the resultant product was washed three times with water and diethyl ether. Then, the obtained organic layer was dried over MgSO₄, and then dried under reduced pressure. Intermediate 271 b was obtained by column chromatography. (yield: about 64%)

### Synthesis of Intermediate 271c

Intermediate 271 b (1.0 eq.), 4-cyclohexylaniline (1.2 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Intermediate 271c was obtained by column chromatography. (yield: about 70%)

### Synthesis of Compound 271

Intermediate 271c (1.0 eq.), 2-chloro-5,9,9-trimethyl-9H-fluorene (1.0 eq), tris(dibenzylideneacetone)dipalladium(0) (0.05 eq.), tri-tert-butylphosphine (0.10 eq.), and sodium tert-butoxide (1.5 eq.) were dissolved in about 50 mL of toluene, and then the resultant mixture was stirred in a nitrogen atmosphere at about 80° C. for about 1 hour. When the reaction was completed, the resultant product was washed three times with water and diethyl ether. Then, the obtained organic layer was dried over MgSO₄, and then dried under reduced pressure. Compound 271 was obtained by column chromatography. (yield: about 73%)

1H NMR and MS/FAB of the compounds synthesized according to Synthetic Examples were listed in Table 1.

TABLE 1

| | | MS/FAB | |
|---|---|---|---|
| Compound | 1H NMR (δ) | Calc | Found |
| Compound | 8.25 (d, 2H), 7.86(d, 1H), 7.79(d, 2H), 7.63(t, | 685.37 | 685.32 |
| 1 | 2H), 7.55(d, 2H), 7.46(t, 3H), 7.45 (d, 1H), | | |
| | 7.33(s, 1H), 7.25-7.23 (m, 9H), 7.08-7.06 (m, | | |
| | 4H), 2.72(m, 1H), 2.58 (s, 3H), 1.85-1.60(m, | | |
| | 4H), 1.69(s, 6H), 1.53-1.43 (m, 6H) | | |
| Compound | 8.25 (d, 2H), 7.86(d, 1H), 7.79(d, 2H), 7.63(t, | 809.40 | 809.35 |
| 6 | 2H), 7.55(d, 2H), 7.46(t, 3H), 7.45 (d, 1H), | | |
| | 7.33(s, 1H), 7.45-7.06 (m, 23H), 2.72(m, 1H), | | |
| | 2.58 (s, 3H), 1.85-1.60(m, 4H), 1.53-1.43 (m, | | |
| | 6H) | | |
| Compound | 8.25-8.24(m, 3H), 7.79 (d, 5H), 7.63-7.41(m, | 796.38 | 796.24 |
| 27 | 16H), 7.27-7.24 (m, 9H), 7.08-7.06 (m, 4H), | | |
| | 2.72(m, 1H), 1.85-1.60(m, 4H), 1.53-1.43 (m, | | |
| | 6H) | | |
| Compound | 8.25 (d, 2H), 8.03(s, 1H), 7.79 (d, 4H), 7.63- | 733.33 | 733.26 |
| 42 | 7.55 (m, 7H), 7.47-7.41 (m, 7H), 7.27-7.25 (m, | | |
| | 6H), 7.08-7.06 (m, 4H), 6.91 (d, 1H), 2.62 (m, | | |
| | 1H), 2.19 (m, 1H), 1.82-1.54 (m, 3H), 1.79-1.31 | | |
| | (m, 6H) | | |
| Compound | 8.25 (d, 2H), 7.89 (d, 1H), 7.79 (d, 2H), 7.63 (t, | 751.42 | 751.34 |
| 63 | 2H), 7.55 (d, 2H), 7.46 (t, 3H), 7.33-7.25 (m, | | |
| | 8H), 7.14-7.10 (m, 4H), 7.00 (d, 2H), 2.92 (s, | | |
| | 3H), 2.27 (s, 3H), 2.05-1.99 (m, 6H), 1.87-1.69 | | |
| | (m, 15H) | | |
| Compound | 8.25 (d, 2H), 7.86 (d, 1H), 7.79 (d, 2H), 7.63 (d, | 809.40 | 809.29 |
| 96 | 2H), 7.55 (t, 1H), 7.46-7.41 (m, 4H), 7.33 (s, | | |
| | 1H), 7.26-7.06 (m, 24H), 2.72(m, 1H), 2.58 (s, | | |
| | 3H), 1.8-1.43 (m, 10H) | | |
| Compound | 8.25 (d, 2H), 7.96 (d, 1H), 7.79-7.74 (m, 5H), | 749.31 | 749.23 |
| 137 | 7.67-7.63 (m, 4H), 7.55-7.41 (m, 9H), 7.27- | | |
| | 7.25 (m, 5H), 7.18-7.17 (m, 2H), 7.08-7.06 (m, | | |
| | 4H), 2.62 (m, 1H), 2.19 (m, 1H), 1.79-1.54 (m, | | |
| | 3H), 1.56-1.31 (m, 6H) | | |
| Compound | 8.25 (d, 2H), 7.89 (d, 1H), 7.79 (d, 2H), 7.63 (d, | 737.40 | 737.32 |
| 151 | 2H), 7.55 (t, 1H), 7.46-7.41 (m, 4H), 7.33 (s, | | |
| | 1H), 7.27-7.10 (m, 12H), 7.00 (d, 2H), 2.58 (s, | | |
| | 3H), 2.05-1.99 (m, 6H), 1.87-1.69 (m, 15H) | | |
| Compound | 8.25 (d, 2H), 8.10 (d, 1H), 8.03 (s, 1H), 7.79 (d, | 673.33 | 673.21 |
| 193 | 2H), 7.63 (d, 2H), 7.55 (d, 1H), 7.46-7.25 (m, | | |
| | 9H), 7.15-7.06 (m, 6H), 6.97-6.89 (m, 2H), 2.72 | | |
| | (m, 1H), 2.58 (s, 3H), 2.27 (s, 3H), 1.85-1.60 | | |
| | (m, 4H), 1.53-1.44 (m, 6H) | | |
| Compound | 8.25 (d, 2H), 8.10 (d, 1H), 7.86 (d, 1H), 7.79- | 883.42 | 883.40 |
| 217 | 7.76 (m, 5H), 7.76-7.53 (m, 3H), 7.46-7.06 (m, | | |
| | 30H), 2.62 (m, 1H), 2.19 (m, 1H), 1.82-1.54 (m, | | |
| | 3H), 1.56-1.21 (m, 6H) | | |
| Compound | 8.25 (d, 2H), 7.86 (d, 1H), 7.63 (d, 2H), 7.55 (d, | 690.40 | 690.28 |
| 271 | 2H), 7.45 (d, 1H), 7.33 (s, 1H), 7.27-7.16 (m, | | |
| | 9H), 7.08-7.06 (m, 4H), 2.72 (m, 1H), 2.58 (s, | | |
| | 3H), 1.85-1.60 (m, 4H), 1.69 (s, 6H), 1.53-1.43 | | |
| | (m, 6H) | | |

### 1. Manufacture and Evaluation of Light-Emitting Element Containing Amine Compound Manufacture of Light-Emitting Element

A light-emitting element containing an amine compound according to an Example in a hole transport layer was manufactured as follows. The light-emitting elements according to Examples 1 to 11 were manufactured by utilizing the amine compounds of Compounds 1, 6, 27, 42, 63, 96, 137, 151, 193, 217, and 271, which are the above-described Example Compounds, as the hole transport layer materials. The light-emitting elements according to Comparative Examples 1 to 7 correspond to the light-emitting elements manufactured by utilizing Comparative Example Compounds A, B, C, D, E, F, and G as the hole transport layer materials.

### Example Compounds

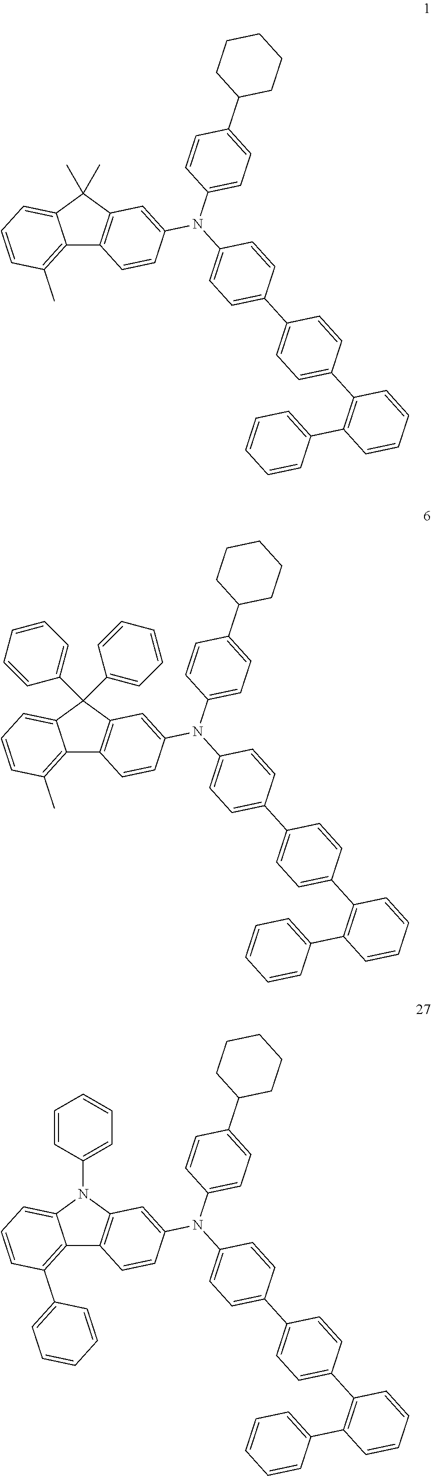 [SMILES: Cc1cccc2c1-c1ccc(N(c3ccc(-c4ccc(-c5ccccc5-c5ccccc5)cc4)cc3)c3ccc(C4CCCCC4)cc3)cc1C2(C)C ; Cc1cccc2c1-c1ccc(N(c3ccc(-c4ccc(-c5ccccc5-c5ccccc5)cc4)cc3)c3ccc(C4CCCCC4)cc3)cc1C2(c1ccccc1)c1ccccc1 ; c1ccc(-c2ccccc2-c2ccc(-c3ccc(N(c4ccc(C5CCCCC5)cc4)c4ccc5c6c(-c7ccccc7)cccc6n(-c6ccccc6)c5c4)cc3)cc2)cc1]

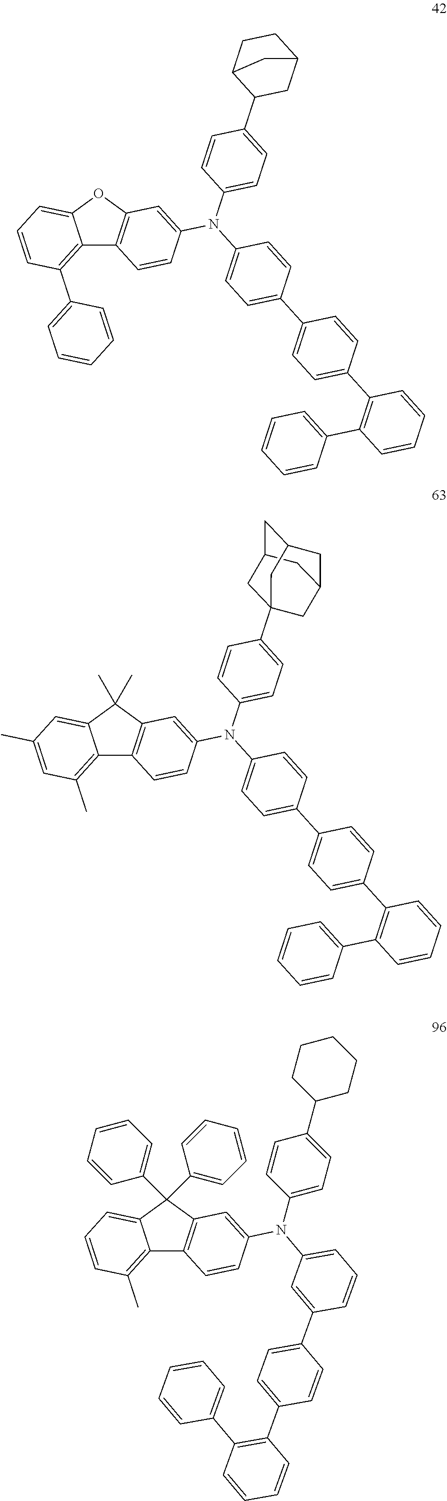 [SMILES: c1ccc(-c2ccccc2-c2ccc(-c3ccc(N(c4ccc(C5CC6CCC5C6)cc4)c4ccc5c(c4)oc4cccc(-c6ccccc6)c45)cc3)cc2)cc1 ; Cc1cc(C)c2c(c1)C(C)(C)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cccc2c1-c1ccc(N(c3ccc(C4CCCCC4)cc3)c3cccc(-c4ccc(-c5ccccc5-c5ccccc5)cc4)c3)cc1C2(c1ccccc1)c1ccccc1]

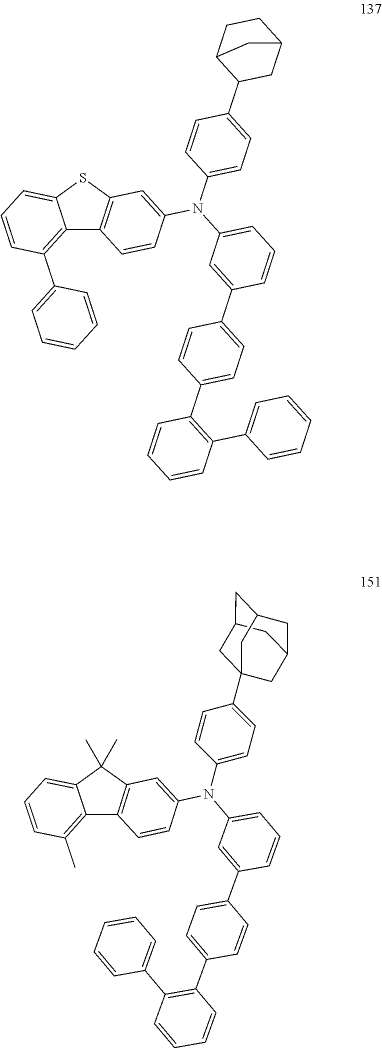 [SMILES: c1ccc(-c2ccccc2-c2ccc(-c3cccc(N(c4ccc(C5CC6CCC5C6)cc4)c4ccc5c(c4)sc4cccc(-c6ccccc6)c45)c3)cc2)cc1 ; Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(C)C]

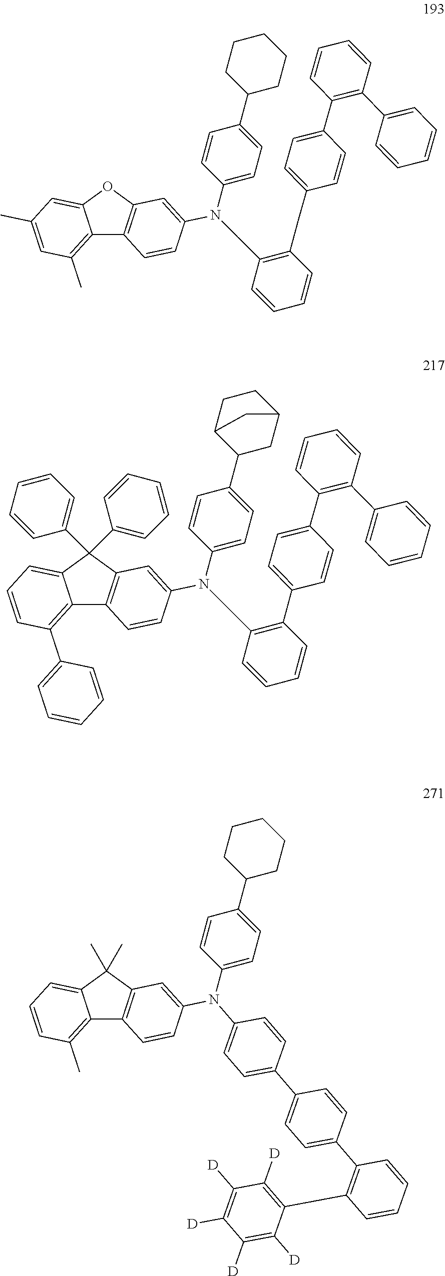 [SMILES: Cc1cc(C)c2c(c1)oc1cc(N(Cc3ccccc3Cc3ccc(-c4ccccc4-c4ccccc4)cc3)c3ccc(C4CCCCC4)cc3)ccc12 ; c1ccc(-c2ccccc2-c2ccc(-c3ccccc3CN(c3ccc(C4CC5CCC4C5)cc3)c3ccc4c(c3)C(c3ccccc3)(c3ccccc3)c3cccc(-c5ccccc5)c3-4)cc2)cc1 ; [2H]c1c([2H])c([2H])c(Cc2ccccc2-c2ccc(-c3ccc(N(c4ccc(C5CCCCC5)cc4)c4ccc5c(c4)C(C)(C)c4cccc(C)c4-5)cc3)cc2)c([2H])c1[2H]]

### Comparative Example Compounds

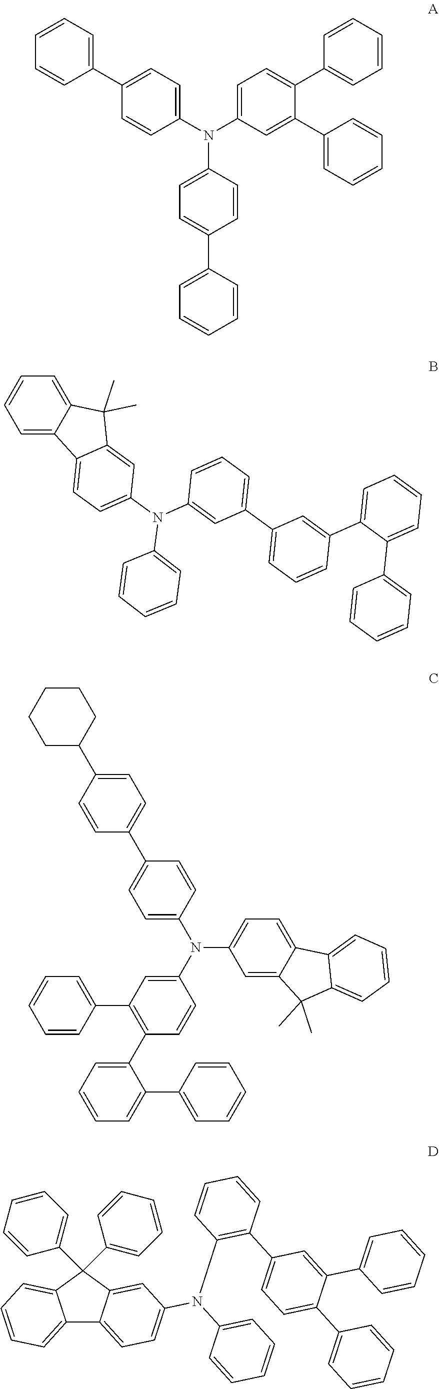 [SMILES: c1ccc(-c2ccc(N(c3ccc(-c4ccccc4)cc3)c3ccc(-c4ccccc4)c(-c4ccccc4)c3)cc2)cc1 ; CC1(C)c2ccccc2-c2ccc(N(c3ccccc3)c3cccc(-c4cccc(-c5ccccc5-c5ccccc5)c4)c3)cc21 ; CC1(C)c2ccccc2-c2ccc(N(c3ccc(-c4ccc(C5CCCCC5)cc4)cc3)c3ccc(-c4ccccc4-c4ccccc4)c(-c4ccccc4)c3)cc21 ; c1ccc(-c2ccc(-c3ccccc3N(c3ccccc3)c3ccc4c(c3)C(c3ccccc3)(c3ccccc3)c3ccccc3-4)cc2-c2ccccc2)cc1]

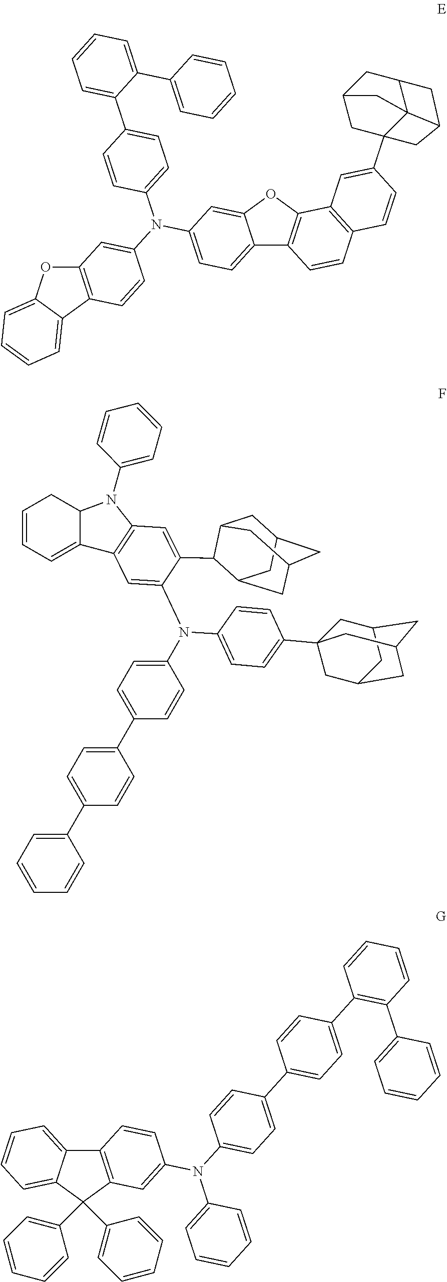 [SMILES: c1ccc(-c2ccccc2-c2ccc(N(c3ccc4c(c3)oc3ccccc34)c3ccc4c(c3)oc3c5cc(C67CC8CC9CC(C6)C97C8)ccc5ccc43)cc2)cc1 ; C1=CCC2C(=C1)c1cc(N(c3ccc(-c4ccc(-c5ccccc5)cc4)cc3)c3ccc(C45CC6CC(CC(C6)C4)C5)cc3)c(C3C4CC5CC(C4)CC3C5)cc1N2c1ccccc1 ; c1ccc(-c2ccccc2-c2ccc(-c3ccc(N(c4ccccc4)c4ccc5c(c4)C(c4ccccc4)(c4ccccc4)c4ccccc4-5)cc3)cc2)cc1]

### Example 1

A glass substrate (made by Corning Co. Ltd), on which an ITO electrode of about 15 Ω/cm² (1,200 Å) was formed as an anode, was cut to a size of about 50 mm×50 mm×0.7 mm, cleansed by ultrasonic waves utilizing isopropyl alcohol and pure water for about five minutes each, and then irradiated with ultraviolet rays for about 30 minutes and exposed to ozone to be cleansed. Then, the glass substrate was mounted on a vacuum deposition apparatus.

NPD was deposited on the anode to form a hole injection layer having a thickness of about 300 Å. Then, Example Compound 1 was deposited on the hole injection layer to form a hole transport layer having a thickness of about 200 Å, and then CzSi was deposited on the hole transport layer to form a light-emitting auxiliary layer having a thickness of about 100 Å.

HT+ET (a host), a phosphorescent sensitizer (PS), and a t-DABNA boron dopant were co-deposited on the light-emitting auxiliary layer at a weight ratio of about 42:42:15:1 to form a light-emitting layer having a thickness of about 200 Å. Sequentially, TSPO1 was deposited on the light-emitting layer to form a hole-blocking layer having a thickness of about 200 Å, and then TPBi was deposited on the hole-blocking layer to form an electron transport layer having a thickness of about 300 Å. Then, LiF was deposited on the electron transport layer to form an electron injection layer having a thickness of about 10 Å, and Al was deposited on the electron injection layer to form a cathode having a thickness of about 3000 Å.

The compounds utilized in manufacturing the light-emitting elements according to Examples and Comparative Examples were disclosed herein.

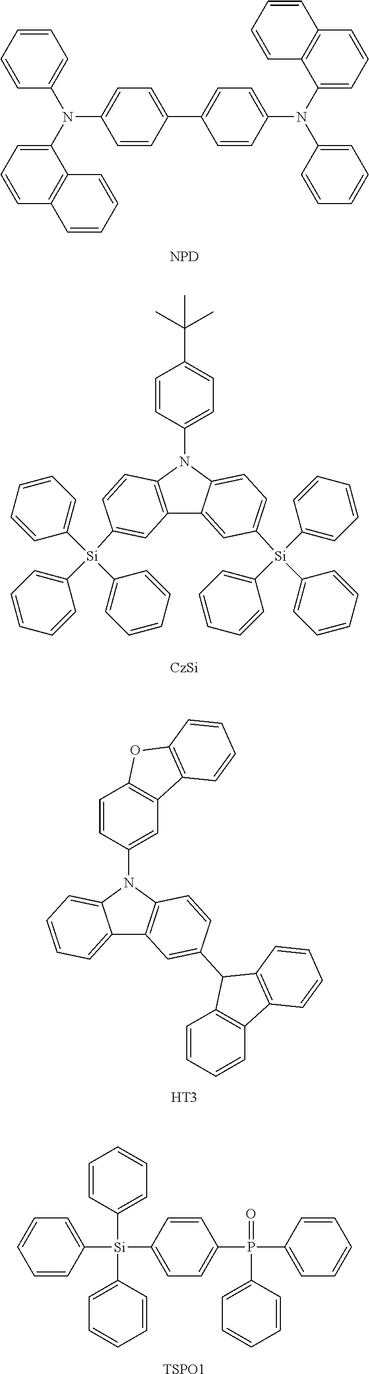

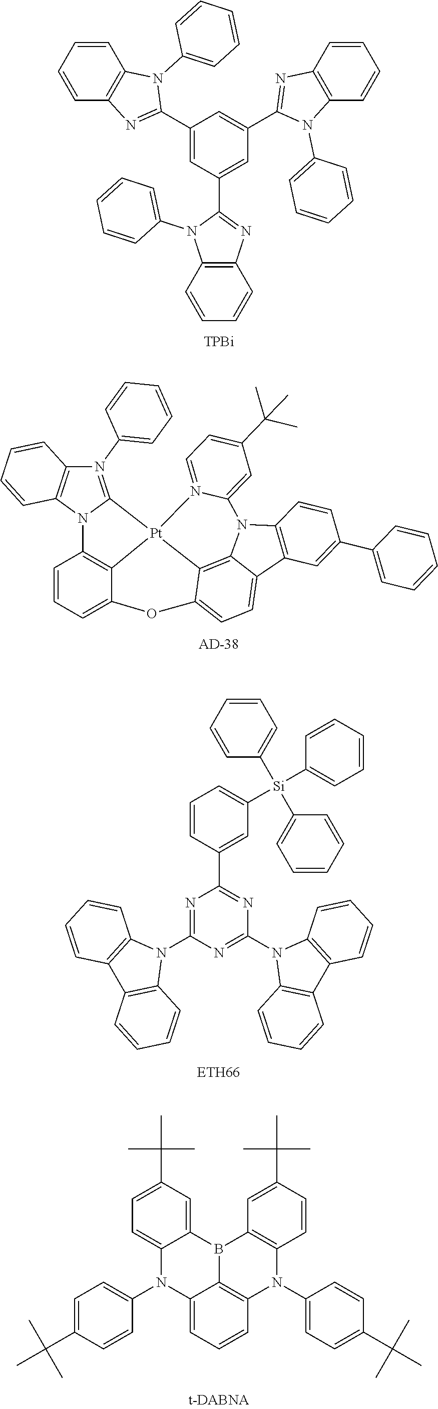

### Example 2

Compared with Example 1, a light-emitting element was manufactured in substantially the same manner as in Example 1, except that Example Compound 6 was utilized in place of Example Compound 1 when forming a hole transport layer. Example 3

Compared with Example 1, a light-emitting element was manufactured in substantially the same manner as in Example 1, except that Example Compound 27 was utilized in place of Example Compound 1 when forming a hole transport layer.

### Example 4

Compared with Example 1, a light-emitting element was manufactured in substantially the same manner as in Example 1, except that Example Compound 42 was utilized in place of Example Compound 1 when forming a hole transport layer.

### Example 5

Compared with Example 1, a light-emitting element was manufactured in substantially the same manner as in Example 1, except that Example Compound 63 was utilized in place of Example Compound 1 when forming a hole transport layer.

### Example 6

Compared with Example 1, a light-emitting element was manufactured in substantially the same manner as in Example 1, except that Example Compound 96 was utilized in place of Example Compound 1 when forming a hole transport layer.

### Example 7

Compared with Example 1, a light-emitting element was manufactured in substantially the same manner as in Example 1, except that Example Compound 137 was utilized in place of Example Compound 1 when forming a hole transport layer. Example 8

Compared with Example 1, a light-emitting element was manufactured in substantially the same manner as in Example 1, except that Example Compound 151 was utilized in place of Example Compound 1 when forming a hole transport layer.

### Example 9

Compared with Example 1, a light-emitting element was manufactured in substantially the same manner as in Example 1, except that Example Compound 193 was utilized in place of Example Compound 1 when forming a hole transport layer.

### Example 10

Compared with Example 1, a light-emitting element was manufactured in substantially the same manner as in Example 1, except that Example Compound 217 was utilized in place of Example Compound 1 when forming a hole transport layer.

### Example 11

Compared with Example 1, a light-emitting element was manufactured in substantially the same manner as in Example 1, except that Example Compound 271 was utilized in place of Example Compound 1 when forming a hole transport layer.

### Comparative Example 1

Compared with Example 1, a light-emitting element was manufactured in substantially the same manner as in Example 1, except that Comparative Example Compound A was utilized in place of Example Compound 1 when forming a hole transport layer.

### Comparative Example 2

Compared with Example 1, a light-emitting element was manufactured in substantially the same manner as in Example 1, except that Comparative Example Compound B was utilized in place of Example Compound 1 when forming a hole transport layer.

### Comparative Example 3

Compared with Example 1, a light-emitting element was manufactured in substantially the same manner as in Example 1, except that Comparative Example Compound C was utilized in place of Example Compound 1 when forming a hole transport layer.

### Comparative Example 4

Compared with Example 1, a light-emitting element was manufactured in substantially the same manner as in Example 1, except that Comparative Example Compound D was utilized in place of Example Compound 1 when forming a hole transport layer.

### Comparative Example 5

Compared with Example 1, a light-emitting element was manufactured in substantially the same manner as in Example 1, except that Comparative Example Compound E was utilized in place of Example Compound 1 when forming a hole transport layer.

### Comparative Example 6

Compared with Example 1, a light-emitting element was manufactured in substantially the same manner as in Example 1, except that Comparative Example Compound F was utilized in place of Example Compound 1 when forming a hole transport layer. Comparative Example 7

Compared with Example 1, a light-emitting element was manufactured in substantially the same manner as in Example 1, except that Comparative Example Compound G was utilized in place of Example Compound 1 when forming a hole transport layer.

### Property Evaluation of Light-Emitting Element

A driving voltage (V), luminous efficiency (cd/A), emission wavelength (m) and lifespan ratio (T95) of the manufactured light-emitting elements were listed in Table 2.

In each of the light-emitting elements according to Examples and Comparative Examples, the driving voltage (V), the luminous efficiency (cd/A), the emission wavelength (nm), and the lifespan (hr @100 mA/cm2) were each measured by utilizing Keithley MU 236 and a luminance meter PR650, and the results are listed in Table 2. The luminous efficiency was measured at a current density of about 50 mA/cm².

When the driving voltage (V) is low, power efficiency is high even when utilizing an element or a material having the same quantum efficiency as when the driving voltage is high. The time taken for the luminance of the element according to Comparative Example 1 to decrease to about 95% of an initial luminance was defined as 1 of a lifespan ratio, and relative values were calculated with respect to the lifespan ratio of the light-emitting element according to Comparative Example 1, and the results were listed.

TABLE 2

| | | Exiplex host | | Boron | Driving | | Emission | Lifespan |
|---|---|---|---|---|---|---|---|---|
| | HTL (Hole | (HT:ET = | Phosphorescent | dopant | voltage | Efficiency | wavelength | (T95) |
| | transport layer) | 5:5) | sensitizer | (t-DABNA) | (V) | (cd/A) | (nm) | ratio |
| Example 1 | Compound 1 | HT3/ETH66 | AD-38 | t-DABNA | 4.12 | 24.8 | 460 | 3.5 |
| Example 2 | Compound 6 | HT3/ETH66 | AD-38 | t-DABNA | 4.10 | 23.9 | 461 | 3.4 |
| Example 3 | Compound 27 | HT3/ETH66 | AD-38 | t-DABNA | 4.20 | 23.5 | 459 | 3.3 |
| Example 4 | Compound 42 | HT3/ETH66 | AD-38 | t-DABNA | 4.52 | 22.8 | 460 | 3.1 |
| Example 5 | Compound 63 | HT3/ETH66 | AD-38 | t-DABNA | 4.25 | 22.5 | 462 | 3.3 |
| Example 6 | Compound 96 | HT3/ETH66 | AD-38 | t-DABNA | 4.32 | 23.6 | 461 | 3.4 |
| Example 7 | Compound 137 | HT3/ETH66 | AD-38 | t-DABNA | 4.55 | 23.1 | 460 | 3.0 |
| Example 8 | Compound 151 | HT3/ETH66 | AD-38 | t-DABNA | 4.35 | 23.3 | 461 | 2.9 |
| Example 9 | Compound 193 | HT3/ETH66 | AD-38 | t-DABNA | 4.60 | 21.2 | 461 | 2.7 |
| Example 10 | Compound 217 | HT3/ETH66 | AD-38 | t-DABNA | 4.62 | 21.8 | 460 | 2.5 |
| Example 11 | Compound 271 | HT3/ETH66 | AD-38 | t-DABNA | 4.13 | 25.6 | 460 | 2.0 |
| Comparative | Compound A | HT3/ETH66 | AD-38 | t-DABNA | 5.70 | 18.5 | 462 | 1 |
| Example 1 | | | | | | | | |
| Comparative | Compound B | HT3/ETH66 | AD-38 | t-DABNA | 5.52 | 19.3 | 461 | 1.5 |
| Example 2 | | | | | | | | |
| Comparative | Compound C | HT3/ETH66 | AD-38 | t-DABNA | 5.25 | 19.1 | 460 | 1.6 |
| Example 3 | | | | | | | | |
| Comparative | Compound D | HT3/ETH66 | AD-38 | t-DABNA | 5.12 | 18.8 | 463 | 1.4 |
| Example 4 | | | | | | | | |
| Comparative | Compound E | HT3/ETH66 | AD-38 | t-DABNA | 5.43 | 18.0 | 461 | 1.1 |
| Example 5 | | | | | | | | |
| Comparative | Compound F | HT3/ETH66 | AD-38 | t-DABNA | 5.32 | 19.0 | 460 | 1.2 |
| Example 6 | | | | | | | | |
| Comparative | Compound G | HT3/ETH66 | AD-38 | t-DABNA | 5.11 | 18.5 | 462 | 1.3 |
| Example 7 | | | | | | | | |

Referring to the results in Table 2, it can be seen that the light-emitting elements utilizing the amine compound, according to Examples of the present disclosure, as the hole transport layer materials, exhibit, compared with those according to Comparative Examples, a low driving voltage value and exhibit relatively high luminous efficiency and element lifespan, while similarly emitting blue light. The light-emitting elements, according to Examples 1 to 11, each have a driving voltage of about 4.10 V to about 4.62 V, and the light-emitting elements, according to Comparative Examples 1 to 7, each have a driving voltage of about 5.11 V to about 5.70 V. For example, the light-emitting elements, according to Comparative Examples 1 to 7, each have a driving voltage value that is at least about 1.11 times to at most about 1.4 times higher than those according to Examples 1 to 11. Therefore, it is expected that the light-emitting elements, according to Examples 1 to 11, will have high power efficiency than the light-emitting elements according to Comparative Examples 1 to 7. The light-emitting elements, according to Examples 1 to 11, each have an efficiency value of about 21.2 (cd/A) to about 25.6 (cd/A), and the light-emitting elements, according to Comparative Examples 1 to 7, each have an efficiency value of about 18.0 (cd/A) to about 19.3 (cd/A). For example, the light-emitting elements, according to Examples 1 to 11, each have an efficiency value that is about 1.1. times to about 1.42 times higher than those according to Comparative Examples 1 to 7. These are similar to a difference in the driving voltage values, and it seems due to the fact that the driving voltage and efficiency have an inversely proportional relation, in which efficiency increases as the drive voltage decreases. The light-emitting elements, according to Examples 1 to 11, each have a lifespan ratio of about 2.0 to about 3.5, and the light-emitting elements, according to Comparative Examples 1 to 7, each have a lifespan ratio of about 1 to about 1.6. Therefore, the light-emitting elements, according to Examples 1 to 11, each have a lifespan that is at least 1.25 times and at most 3.5 times higher than those of Comparative Examples 1 to 7. In conclusion, the light-emitting elements, according to Examples 1 to 11, each have a lower driving voltage and higher efficiency and lifespan ratio than those according to Comparative Examples 1 to 7.

Example Compounds included in Examples 1 to 11 have a structure in which a first substituent, a second substituent, and a third substituent are connected to a core nitrogen atom, and the first substituent includes a dibenzofuranyl moiety, a dibenzothiophenyl moiety, a carbazole moiety, or a fluorenyl moiety. The second substituent has a structure in which a cyclohexyl group, a bicycloheptanyl group, or an aliphatic cycloalkyl group of an adamantyl group is connected via an arylene linker. The third substituent has a substituted or unsubstituted quarterphenyl structure. In the third substituent, a benzene ring of the quarterphenyl group bonded to the nitrogen atom is named as a first benzene ring, and benzene rings are sequentially named as second, third, and fourth benzene rings from the benzene ring bonded to the first benzene ring. In this case, the third benzene ring is bonded at a para position with respect to the carbon atom, among ring-forming carbon atoms of the second benzene ring, which forms a bond between the first benzene ring and the second benzene ring. In some embodiments, the fourth benzene ring is bonded at an ortho position with respect to the carbon atom among ring-forming carbon atoms of the third benzene ring, which forms a bond with the second benzene ring. When the amine compound according to an Example, which has such a structure, is utilized in the hole transport region, an energy level of HOMO may be variously changed by varying the substituent of the nitrogen atom. Therefore, a hole injection barrier of the hole transport region is variously changed, thereby increasing an exciton generation efficiency inside the light-emitting layer. In some embodiments, a refractive index of the hole transport region is adjusted by varying the substituent that may be substituted for the nitrogen atom included in the amine compound according to an Example, thereby improving external quantum efficiency. As described above, when the amine compound according to an Example is utilized in the hole transport region, the luminous efficiency of the light-emitting element may increase, and the lifespan of the light-emitting element may be improved. As a result, when the amine compound according to an Example is applied to the light-emitting element or the display device, improvement in luminous efficiency and long-lifespan may be achieved.

Comparative Example Compounds A to G included in the light-emitting elements according to Comparative Examples 1 to 7 do not concurrently (e.g., simultaneously) include the first substituent, the second substituent, and the third substituent, each connected to a nitrogen atom. Therefore, it can be seen that the driving voltage is high, luminous efficiency decreases, and lifespan decreases compared with Example Compounds when Comparative Example Compounds are applied to a light-emitting element.

The light-emitting element according to an embodiment may exhibit improved element properties of high efficiency and/or long-lifespan.

The display device according to an embodiment may exhibit improved luminous efficiency properties.

The amine compound according to an embodiment is included in the hole transport region of the light-emitting element and may thus contribute to the achievement of high efficiency and/or long-lifespan.

In the present disclosure, singular expressions may include plural expressions unless the context clearly indicates otherwise. It will be further understood that the terms “comprise(s),” “include(s),” or “have/has” when utilized in the present disclosure, specify the presence of stated features, integers, steps, operations, elements, and/or components, but do not preclude the presence or addition of one or more other features, integers, steps, operations, elements, components, and/or groups thereof. The “/” utilized herein may be interpreted as “and” or as “or” depending on the situation.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

In the present disclosure, when particles (e.g., nanoparticles) are spherical, “size” indicates a particle diameter or an average particle diameter, and when the particles are non-spherical, the “size” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

In present disclosure, “not include a or any ‘component’” “exclude a or any ‘component’”, “‘component’-free”, and/or the like refers to that the “component” not being added, selected, or utilized as a component in a compound/composition/structure, but the “component” of less than a suitable amount may still be included due to other impurities and/or external factors in a composition.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

As utilized herein, the term “and/or” includes any and all combinations of one or more of the associated listed items. Throughout the disclosure, the expression “at least one of a, b or c”, “at least one of a-c”, “at least one of a to c”, “at least one of a, b, and/or c”, “at least one among a to c”, etc., indicates only a, only b, only c, both (e.g., simultaneously) a and b, both (e.g., simultaneously) a and c, both (e.g., simultaneously) b and c, all of a, b, and c, or variations thereof.

In the present specification, “including A or B”, “A and/or B”, etc., represents A or B, or A and B.

The light-emitting device, the display device, the electronic apparatus, the electronic equipment, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments but one or more suitable changes and modifications can be made by one ordinary skilled in the art within the spirit and scope of the present disclosure as hereinafter claimed.

Accordingly, the technical scope of the present disclosure is not intended to be limited to the contents set forth in the detailed description of the specification, but is intended to be defined by the appended claims, and equivalents thereof.

What is claimed is:

1. A light-emitting element comprising:
a first electrode;
a second electrode spaced from the first electrode; and
a functional layer between the first electrode and the second electrode,
wherein the functional layer contains an amine compound represented by Formula 1: 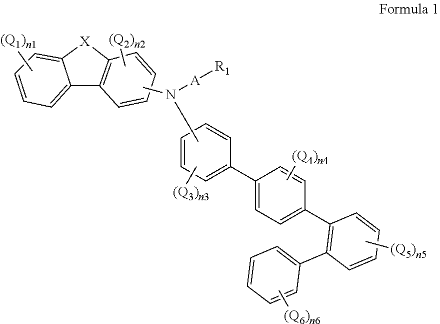
wherein, in Formula 1,
n1, n3, n4, and n5 are each independently an integer of 0 to 4,
n2 is an integer of 0 to 3,
n6 is an integer of 0 to 5,
n1 and n2 are not 0 at the same time,
X is O, S, NR₂ or CR₃R₄,
A is a substituted or unsubstituted arylene group having 6 to 30 ring-forming carbon atoms,
R₁ is a substituted or unsubstituted cycloalkyl group having 3 to 10 ring-forming carbon atoms,
R₂, R₃, and R₄ are each independently a hydrogen atom, a deuterium atom, a substituted or unsubstituted alkyl group having 1 to 30 carbon atoms, or a substituted or unsubstituted aryl group having 6 to 30 ring-forming carbon atoms,
Q₁ and Q₂ are each independently a substituted or unsubstituted alkyl group having 1 to 10 carbon atoms, or a substituted or unsubstituted aryl group having 6 to 30 ring-forming carbon atoms, and
Q₃, Q₄, Q₅, and Q₆ are each independently a hydrogen atom, a deuterium atom, a substituted or unsubstituted alkyl group having 1 to 10 carbon atoms, or a substituted or unsubstituted aryl group having 6 to 30 ring-forming carbon atoms.

2. The light-emitting element of claim 1,
wherein the functional layer comprises a light-emitting layer, a hole transport region between the first electrode and the light-emitting layer, and an electron transport region between the light-emitting layer and the second electrode, and
the hole transport region contains the amine compound represented by Formula 1.

3. The light-emitting element of claim 2,
wherein the hole transport region comprises:
a hole injection layer between the first electrode and the light-emitting layer; and
a hole transport layer between the hole injection layer and the light-emitting layer, and
at least one selected from among the hole injection layer and the hole transport layer contains the amine compound represented by Formula 1.

4. The light-emitting element of claim 3,
wherein the hole transport region further comprises an electron-blocking layer between the hole transport layer and the light-emitting layer, and
at least any one selected from among the hole injection layer, the hole transport layer, and the electron-blocking layer contains the amine compound represented by Formula 1.

5. The light-emitting element of claim 1, wherein the amine compound represented by Formula 1 is a monoamine compound.

6. The light-emitting element of claim 1,
wherein the amine compound represented by Formula 1 is represented by Formula 1-1: 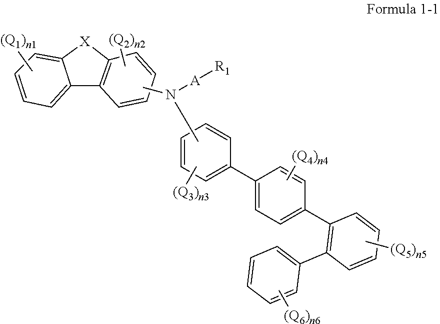
wherein, in Formula 1-1,
n1 to n6, X, A, R₁ to R₄, and Q₁ to Q₆ are the same as defined in the descriptions of Formula 1.

7. The light-emitting element of claim 1,
wherein the amine compound represented by Formula 1 is represented by any one selected from among Formulas 2-1 to 2-3: 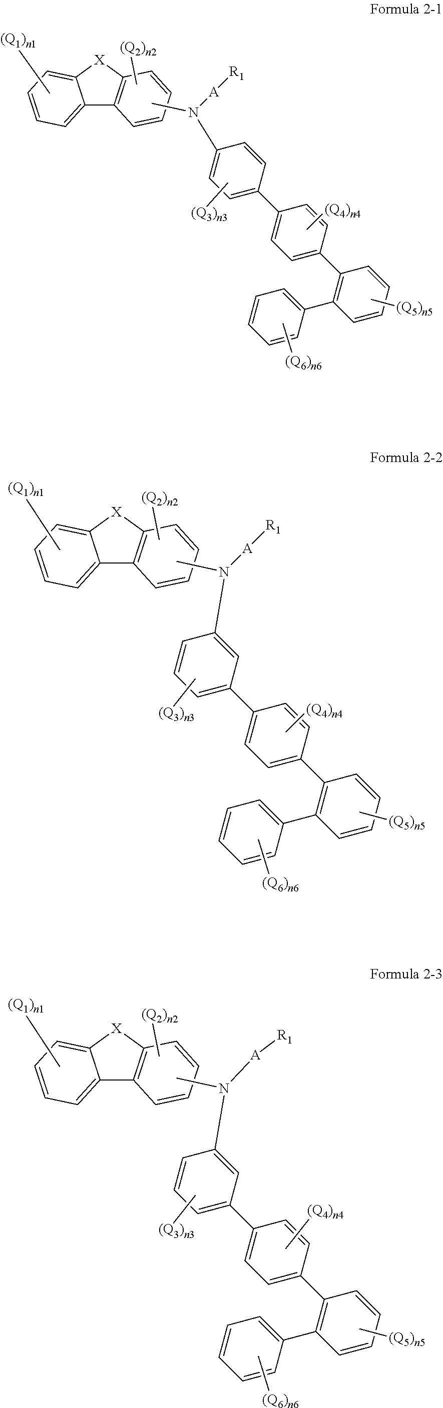
wherein, in Formulas 2-1 to 2-3,
n1 to n6, X, A, R₁ to R₄, and Q₁ to Q₆ are the same as defined in the descriptions of Formula 1.

8. The light-emitting element of claim 1,
wherein the amine compound represented by Formula 1 is represented by any one selected from among Formulas 3-1 to 3-4: 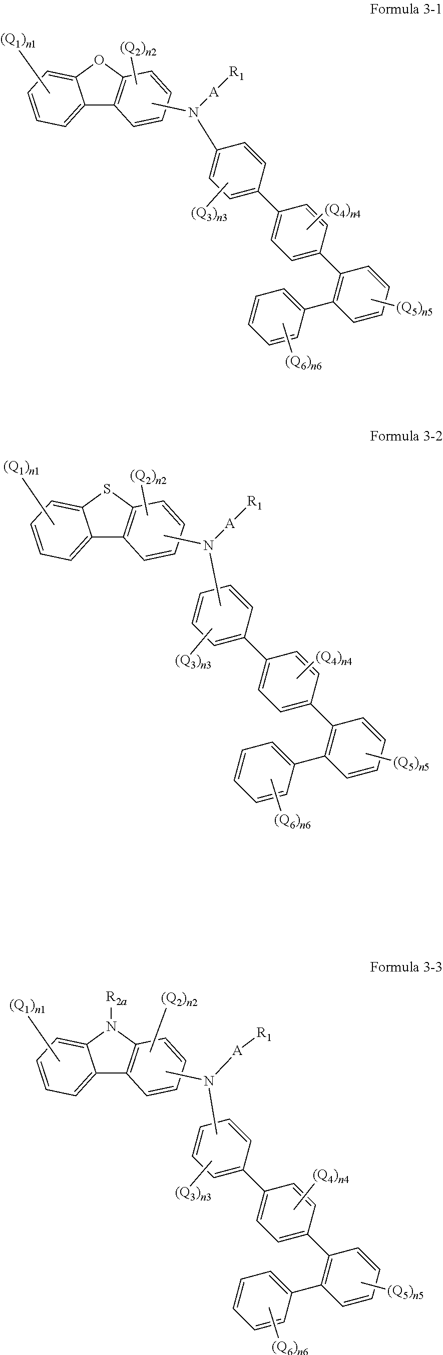 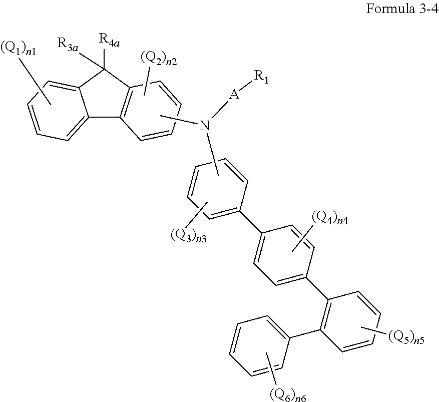
wherein, in Formulas 3-1 to 3-4,
R₂ₐ, R₃ₐ, and R₄ₐ are each independently a hydrogen atom, a deuterium atom,
an unsubstituted alkyl group having 1 to 30 carbon atoms, or an unsubstituted aryl group having 6 to 30 ring-forming carbon atoms, and
n1 to n6, A, R₁ and Q₁ to Q₆ are the same as defined in the descriptions of Formula 1.

9. The light-emitting element of claim 1,
wherein the amine compound represented by Formula 1 is represented by Formula 4: 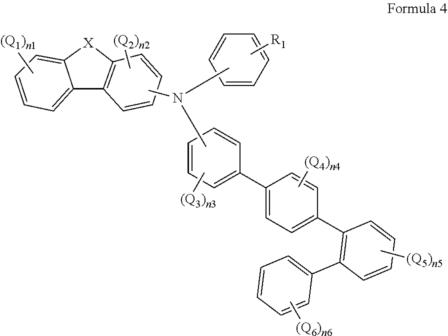
wherein, in Formula 4,
n1 to n6, X, R₁ to R₄, and Q₁ to Q₆ are the same as defined in the descriptions of Formula 1.

10. The light-emitting element of claim 9,
wherein the amine compound represented by Formula 4 is represented by Formula 4-1: 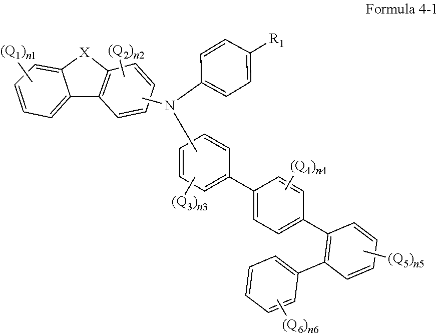
wherein, in Formula 4-1,
n1 to n6, X, R₁ to R₄, and Q₁ to Q₆ are the same as defined in the descriptions of Formula 1.

11. The light-emitting element of claim 1,
wherein, in Formula 1,
R₁ is a substituted or unsubstituted cyclohexyl group, a substituted or unsubstituted bicycloheptanyl group, or a substituted or unsubstituted adamantyl group.

12. The light-emitting element of claim 1,
wherein the amine compound represented by Formula 1 is represented by Formula 5: 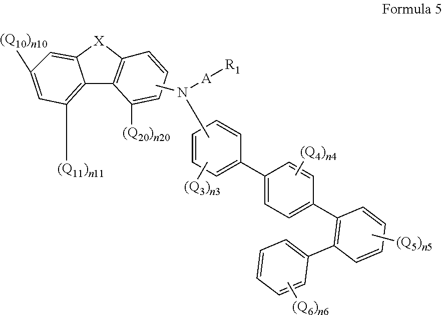
wherein, in Formula 5,
n10, n11, and n20 are each independently 0 or 1,
n10, n11, and n20 are not 0 at the same time,
Q₁ is a substituted or unsubstituted alkyl group having 1 to 10 carbon atoms,
Q₁₁ and Q₁₂ are each independently a substituted or unsubstituted alkyl group having 1 to 10 carbon atoms, or a substituted or unsubstituted aryl group having 6 to 30 ring-forming carbon atoms, and
n3 to n6, X, A, R₁ to R₄, and Q₃ to Q₆ are the same as defined in the descriptions of Formula 1.

13. The light-emitting element of claim 12,
wherein, in Formula 5,
Q₁ is a substituted or unsubstituted methyl group, and
Q₁₁ and Q₂₀ are each independently a substituted or unsubstituted methyl group, or a substituted or unsubstituted phenyl group.

14. The light-emitting element of claim 1,
wherein the amine compound represented by Formula 1 is represented by any one selected from among compounds presented in Compound Group 1:

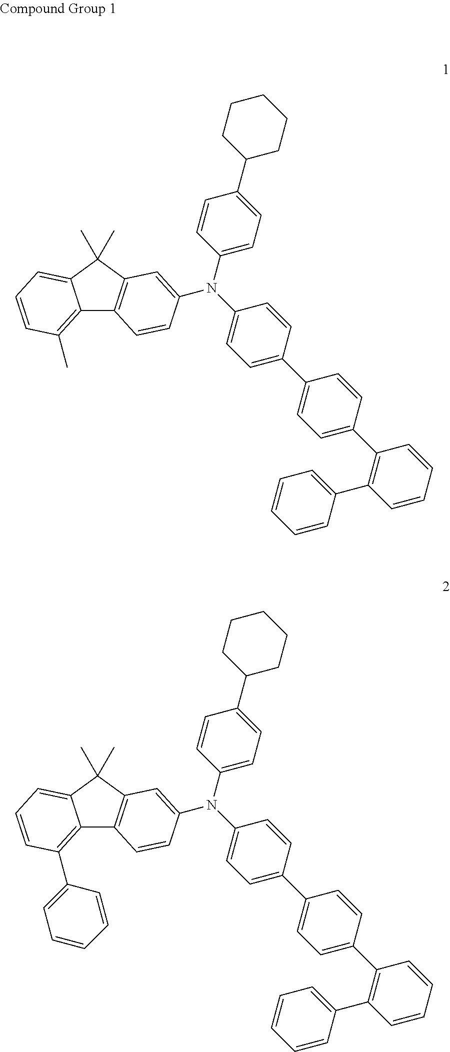 [SMILES: Cc1cccc2c1-c1ccc(N(c3ccc(-c4ccc(-c5ccccc5-c5ccccc5)cc4)cc3)c3ccc(C4CCCCC4)cc3)cc1C2(C)C ; CC1(C)c2cc(N(c3ccc(-c4ccc(-c5ccccc5-c5ccccc5)cc4)cc3)c3ccc(C4CCCCC4)cc3)ccc2-c2c(-c3ccccc3)cccc21]

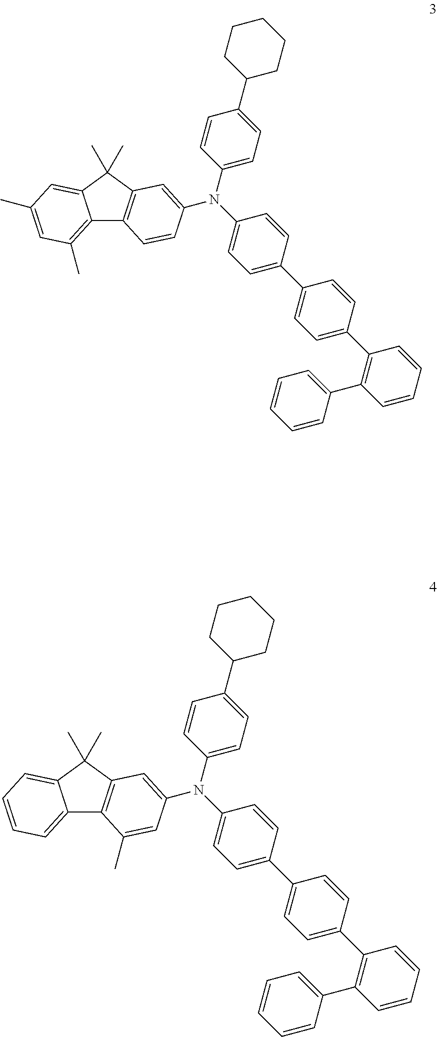 [SMILES: Cc1cc(C)c2c(c1)C(C)(C)c1cc(N(c3ccc(-c4ccc(-c5ccccc5-c5ccccc5)cc4)cc3)c3ccc(C4CCCCC4)cc3)ccc1-2 ; Cc1cc(N(c2ccc(-c3ccc(-c4ccccc4-c4ccccc4)cc3)cc2)c2ccc(C3CCCCC3)cc2)cc2c1-c1ccccc1C2(C)C]

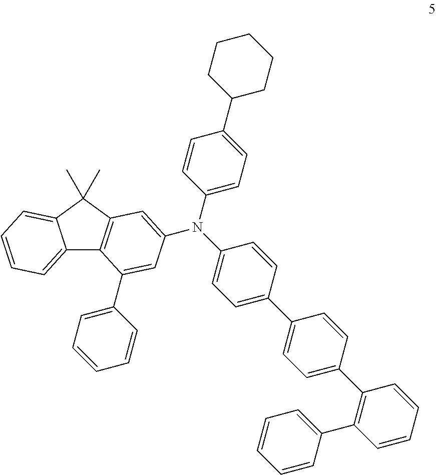 [SMILES: CC1(C)c2ccccc2-c2c(-c3ccccc3)cc(N(c3ccc(-c4ccc(-c5ccccc5-c5ccccc5)cc4)cc3)c3ccc(C4CCCCC4)cc3)cc21]

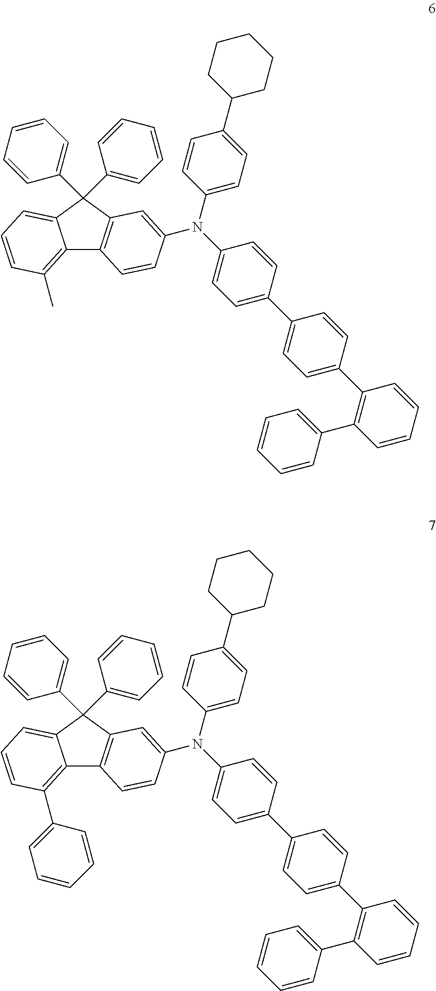 [SMILES: Cc1cccc2c1-c1ccc(N(c3ccc(-c4ccc(-c5ccccc5-c5ccccc5)cc4)cc3)c3ccc(C4CCCCC4)cc3)cc1C2(c1ccccc1)c1ccccc1 ; c1ccc(-c2ccccc2-c2ccc(-c3ccc(N(c4ccc(C5CCCCC5)cc4)c4ccc5c(c4)C(c4ccccc4)(c4ccccc4)c4cccc(-c6ccccc6)c4-5)cc3)cc2)cc1]

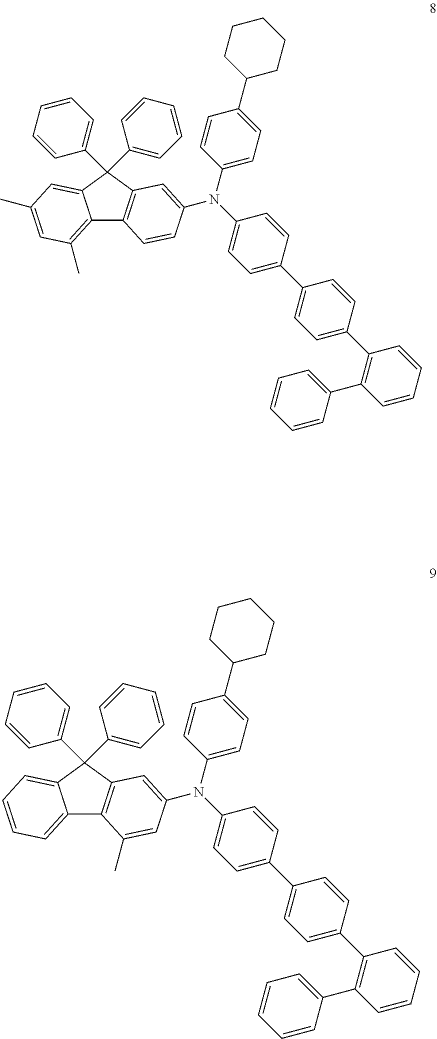 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C4CCCCC4)cc3)ccc1-2 ; Cc1cc(N(c2ccc(-c3ccc(-c4ccccc4-c4ccccc4)cc3)cc2)c2ccc(C3CCCCC3)cc2)cc2c1-c1ccccc1C2(c1ccccc1)c1ccccc1]

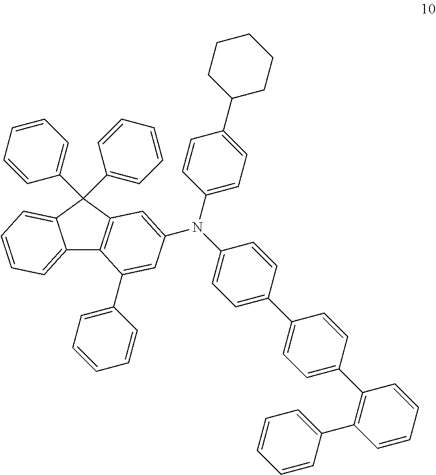 [SMILES: c1ccc(-c2ccccc2-c2ccc(-c3ccc(N(c4ccc(C5CCCCC5)cc4)c4cc(-c5ccccc5)c5c(c4)C(c4ccccc4)(c4ccccc4)c4ccccc4-5)cc3)cc2)cc1]

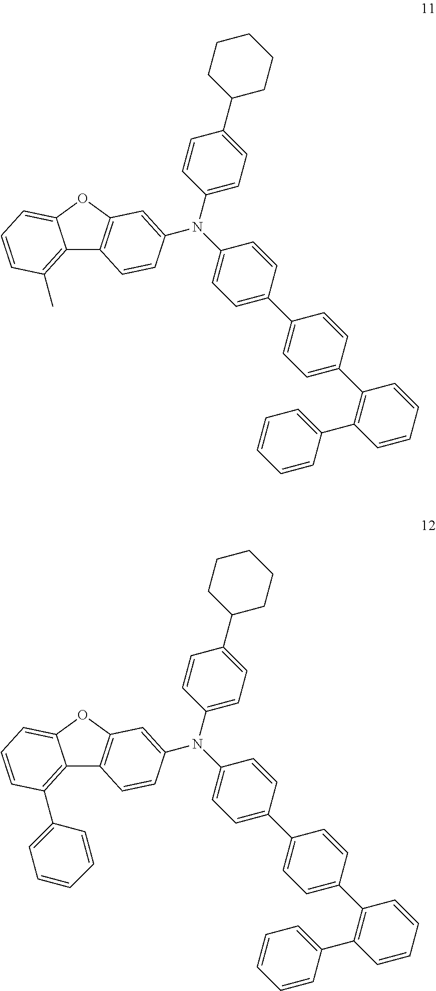 [SMILES: Cc1cccc2oc3cc(N(c4ccc(-c5ccc(-c6ccccc6-c6ccccc6)cc5)cc4)c4ccc(C5CCCCC5)cc4)ccc3c12 ; c1ccc(-c2ccccc2-c2ccc(-c3ccc(N(c4ccc(C5CCCCC5)cc4)c4ccc5c(c4)oc4cccc(-c6ccccc6)c45)cc3)cc2)cc1]

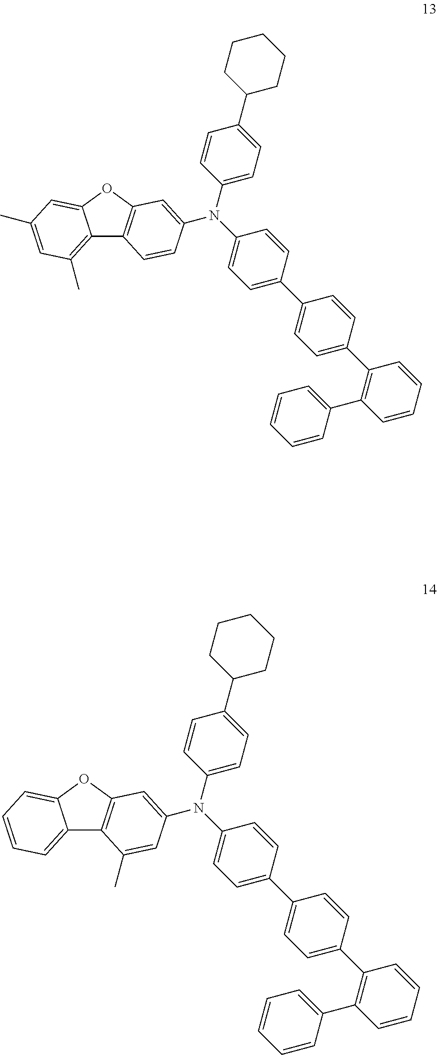 [SMILES: Cc1cc(C)c2c(c1)oc1cc(N(c3ccc(-c4ccc(-c5ccccc5-c5ccccc5)cc4)cc3)c3ccc(C4CCCCC4)cc3)ccc12 ; Cc1cc(N(c2ccc(-c3ccc(-c4ccccc4-c4ccccc4)cc3)cc2)c2ccc(C3CCCCC3)cc2)cc2oc3ccccc3c12]

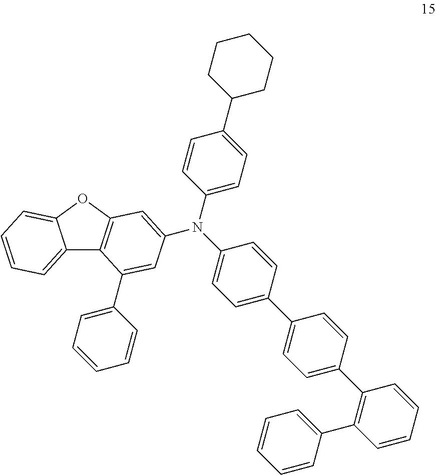 [SMILES: c1ccc(-c2ccccc2-c2ccc(-c3ccc(N(c4ccc(C5CCCCC5)cc4)c4cc(-c5ccccc5)c5c(c4)oc4ccccc45)cc3)cc2)cc1]

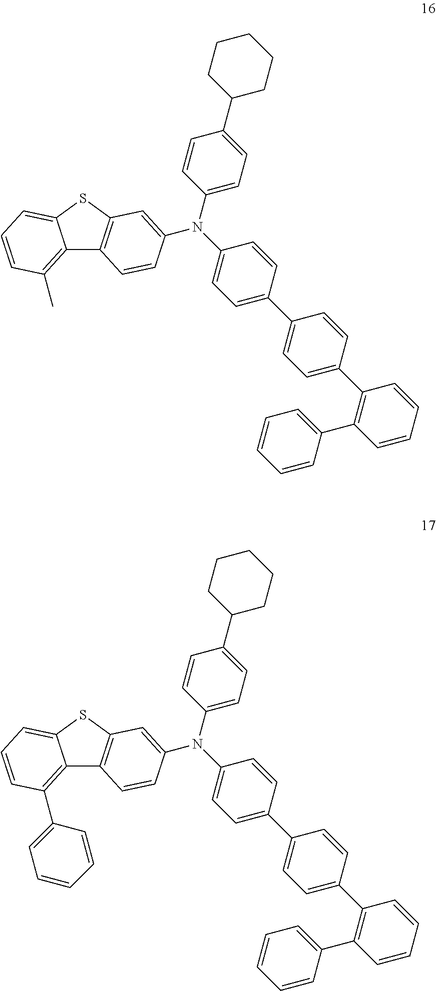 [SMILES: Cc1cccc2sc3cc(N(c4ccc(-c5ccc(-c6ccccc6-c6ccccc6)cc5)cc4)c4ccc(C5CCCCC5)cc4)ccc3c12 ; c1ccc(-c2ccccc2-c2ccc(-c3ccc(N(c4ccc(C5CCCCC5)cc4)c4ccc5c(c4)sc4cccc(-c6ccccc6)c45)cc3)cc2)cc1]

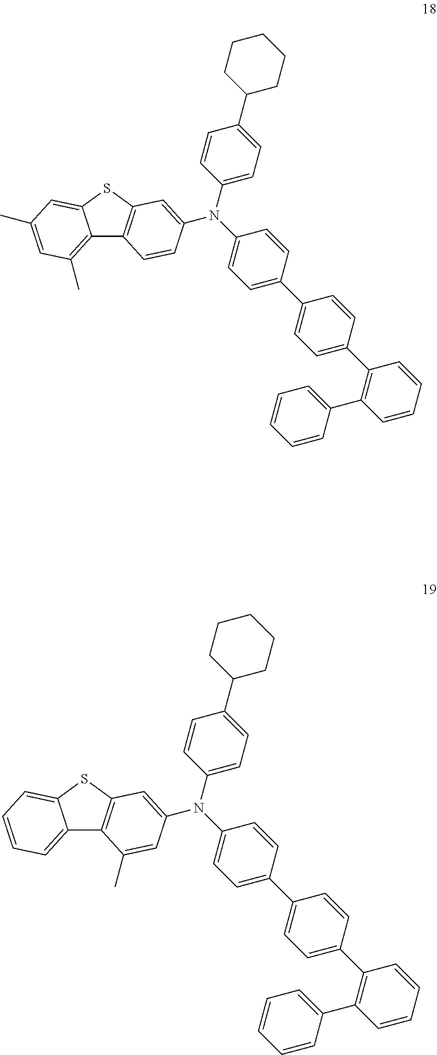 [SMILES: Cc1cc(C)c2c(c1)sc1cc(N(c3ccc(-c4ccc(-c5ccccc5-c5ccccc5)cc4)cc3)c3ccc(C4CCCCC4)cc3)ccc12 ; Cc1cc(N(c2ccc(-c3ccc(-c4ccccc4-c4ccccc4)cc3)cc2)c2ccc(C3CCCCC3)cc2)cc2sc3ccccc3c12]

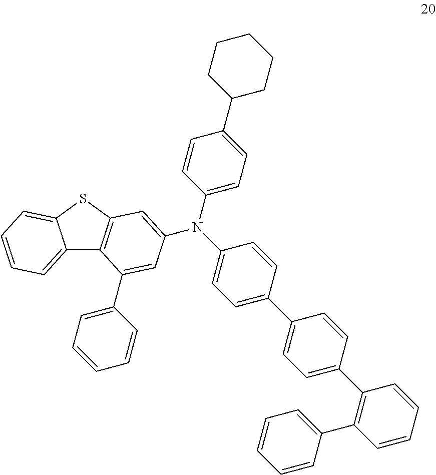 [SMILES: c1ccc(-c2ccccc2-c2ccc(-c3ccc(N(c4ccc(C5CCCCC5)cc4)c4cc(-c5ccccc5)c5c(c4)sc4ccccc45)cc3)cc2)cc1]

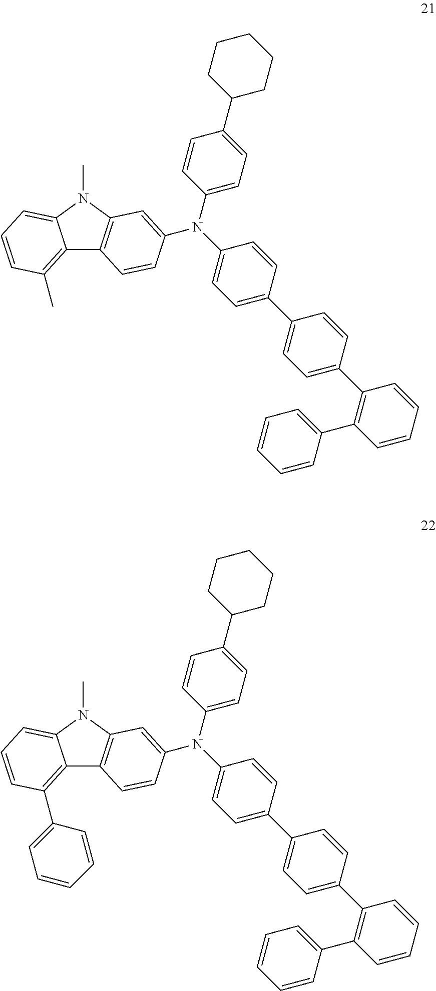 [SMILES: Cc1cccc2c1c1ccc(N(c3ccc(-c4ccc(-c5ccccc5-c5ccccc5)cc4)cc3)c3ccc(C4CCCCC4)cc3)cc1n2C ; Cn1c2cc(N(c3ccc(-c4ccc(-c5ccccc5-c5ccccc5)cc4)cc3)c3ccc(C4CCCCC4)cc3)ccc2c2c(-c3ccccc3)cccc21]

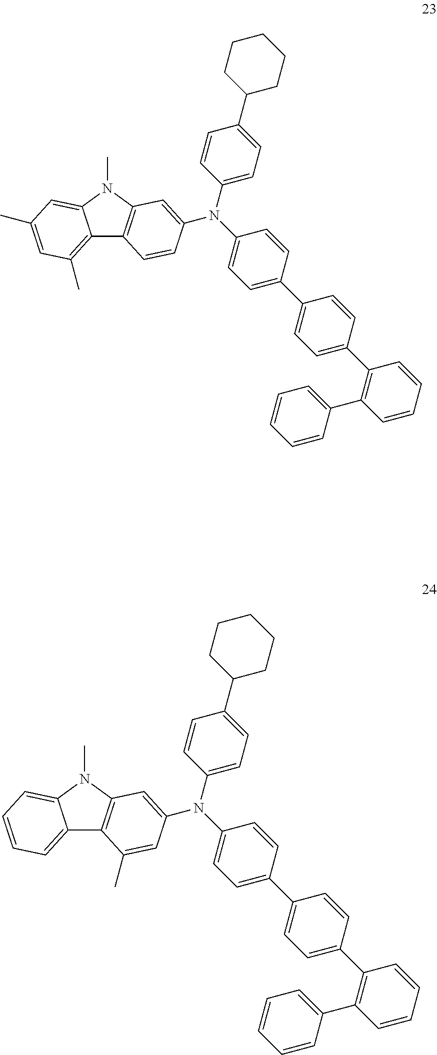 [SMILES: Cc1cc(C)c2c3ccc(N(c4ccc(-c5ccc(-c6ccccc6-c6ccccc6)cc5)cc4)c4ccc(C5CCCCC5)cc4)cc3n(C)c2c1 ; Cc1cc(N(c2ccc(-c3ccc(-c4ccccc4-c4ccccc4)cc3)cc2)c2ccc(C3CCCCC3)cc2)cc2c1c1ccccc1n2C]

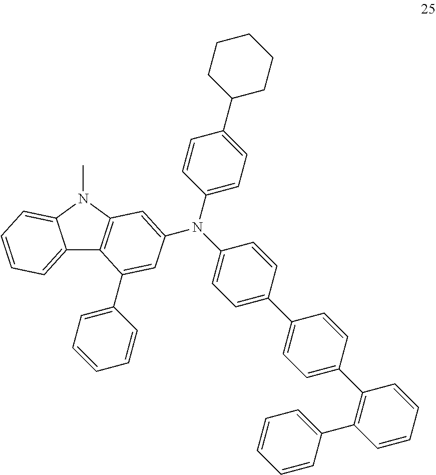 [SMILES: Cn1c2ccccc2c2c(-c3ccccc3)cc(N(c3ccc(-c4ccc(-c5ccccc5-c5ccccc5)cc4)cc3)c3ccc(C4CCCCC4)cc3)cc21]

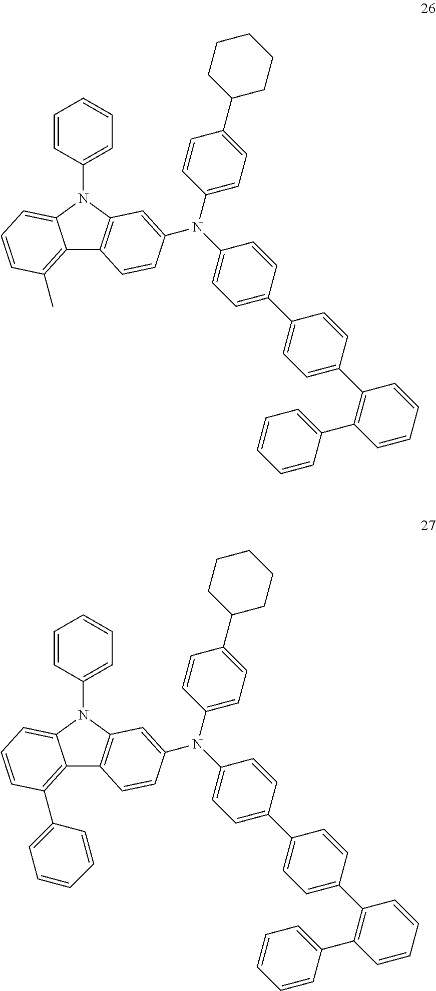 [SMILES: Cc1cccc2c1c1ccc(N(c3ccc(-c4ccc(-c5ccccc5-c5ccccc5)cc4)cc3)c3ccc(C4CCCCC4)cc3)cc1n2-c1ccccc1 ; c1ccc(-c2ccccc2-c2ccc(-c3ccc(N(c4ccc(C5CCCCC5)cc4)c4ccc5c6c(-c7ccccc7)cccc6n(-c6ccccc6)c5c4)cc3)cc2)cc1]

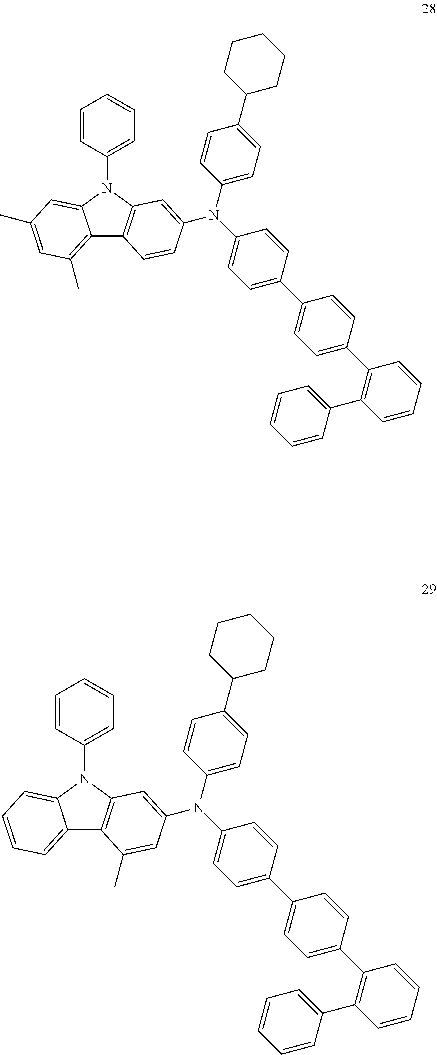 [SMILES: Cc1cc(C)c2c3ccc(N(c4ccc(-c5ccc(-c6ccccc6-c6ccccc6)cc5)cc4)c4ccc(C5CCCCC5)cc4)cc3n(-c3ccccc3)c2c1 ; Cc1cc(N(c2ccc(-c3ccc(-c4ccccc4-c4ccccc4)cc3)cc2)c2ccc(C3CCCCC3)cc2)cc2c1c1ccccc1n2-c1ccccc1]

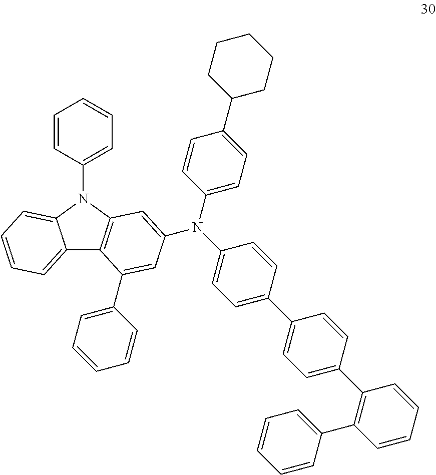 [SMILES: c1ccc(-c2ccccc2-c2ccc(-c3ccc(N(c4ccc(C5CCCCC5)cc4)c4cc(-c5ccccc5)c5c6ccccc6n(-c6ccccc6)c5c4)cc3)cc2)cc1]

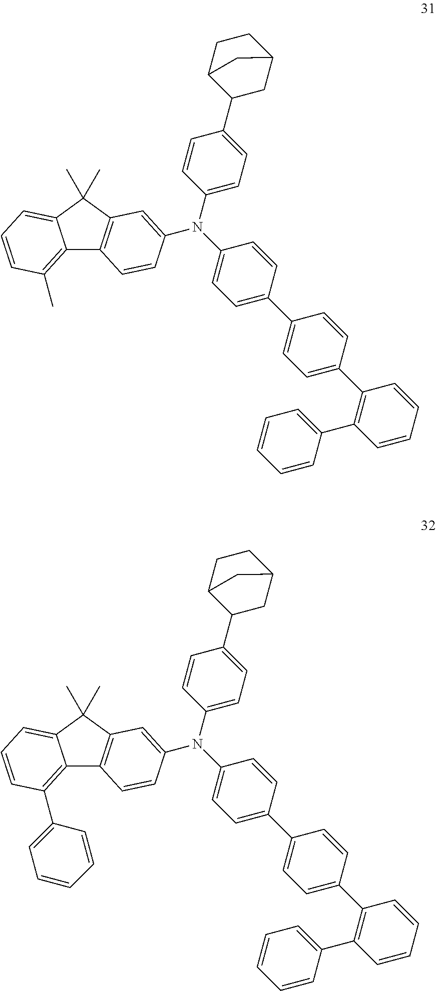 [SMILES: Cc1cccc2c1-c1ccc(N(c3ccc(-c4ccc(-c5ccccc5-c5ccccc5)cc4)cc3)c3ccc(C4CC5CCC4C5)cc3)cc1C2(C)C ; CC1(C)c2cc(N(c3ccc(-c4ccc(-c5ccccc5-c5ccccc5)cc4)cc3)c3ccc(C4CC5CCC4C5)cc3)ccc2-c2c(-c3ccccc3)cccc21]

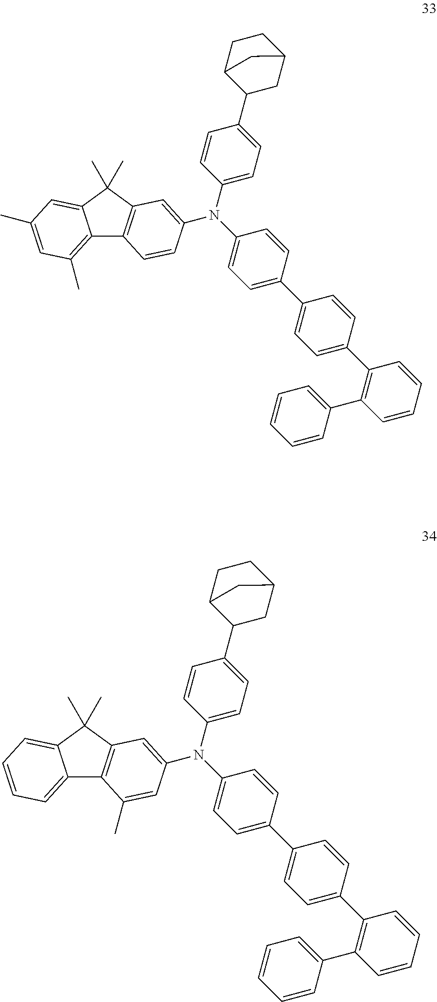 [SMILES: Cc1cc(C)c2c(c1)C(C)(C)c1cc(N(c3ccc(-c4ccc(-c5ccccc5-c5ccccc5)cc4)cc3)c3ccc(C4CC5CCC4C5)cc3)ccc1-2 ; Cc1cc(N(c2ccc(-c3ccc(-c4ccccc4-c4ccccc4)cc3)cc2)c2ccc(C3CC4CCC3C4)cc2)cc2c1-c1ccccc1C2(C)C]

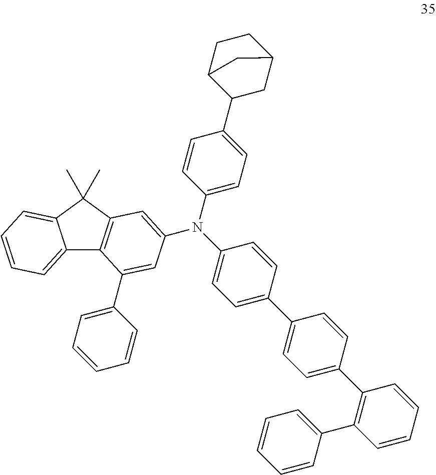 [SMILES: CC1(C)c2ccccc2-c2c(-c3ccccc3)cc(N(c3ccc(-c4ccc(-c5ccccc5-c5ccccc5)cc4)cc3)c3ccc(C4CC5CCC4C5)cc3)cc21]

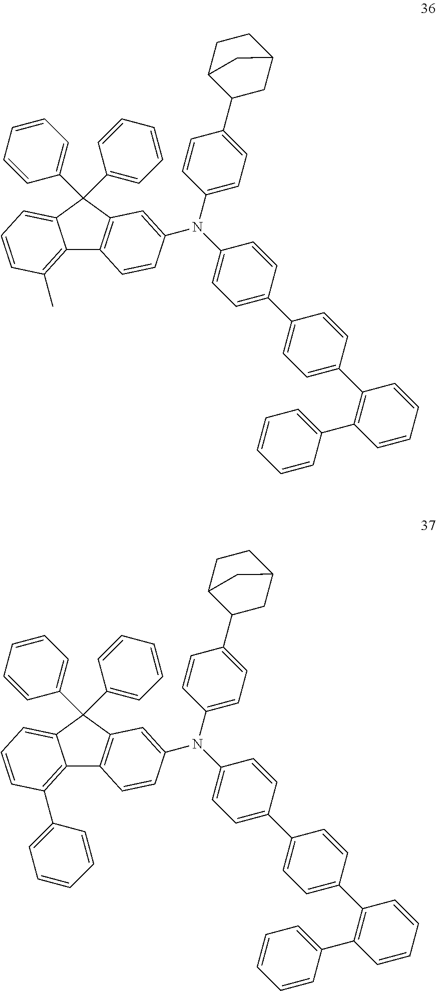 [SMILES: Cc1cccc2c1-c1ccc(N(c3ccc(-c4ccc(-c5ccccc5-c5ccccc5)cc4)cc3)c3ccc(C4CC5CCC4C5)cc3)cc1C2(c1ccccc1)c1ccccc1 ; c1ccc(-c2ccccc2-c2ccc(-c3ccc(N(c4ccc(C5CC6CCC5C6)cc4)c4ccc5c(c4)C(c4ccccc4)(c4ccccc4)c4cccc(-c6ccccc6)c4-5)cc3)cc2)cc1]

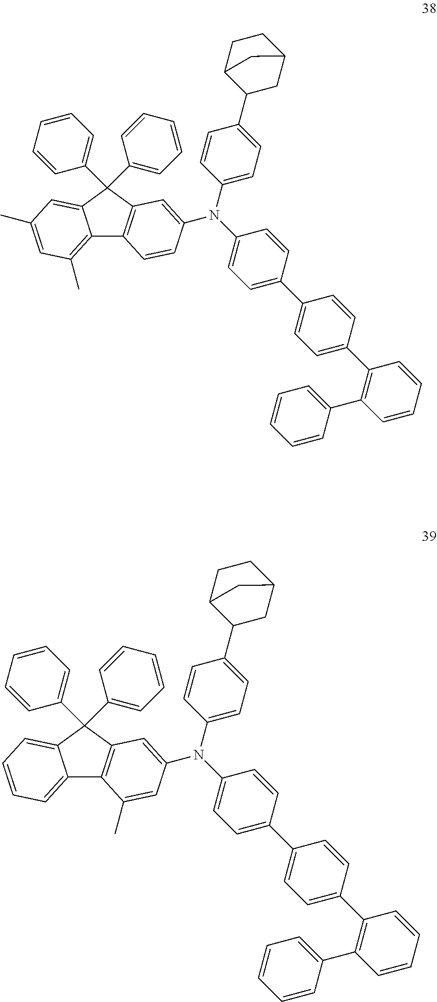 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C4CC5CCC4C5)cc3)ccc1-2 ; Cc1cc(N(c2ccc(-c3ccc(-c4ccccc4-c4ccccc4)cc3)cc2)c2ccc(C3CC4CCC3C4)cc2)cc2c1-c1ccccc1C2(c1ccccc1)c1ccccc1]

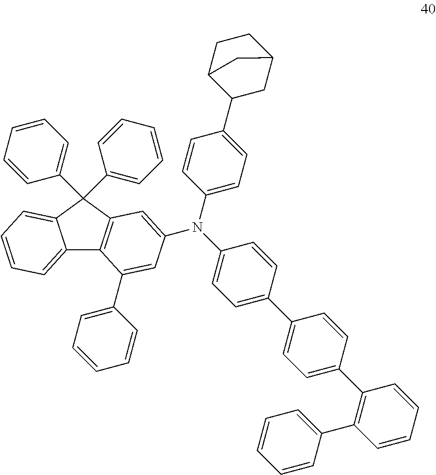 [SMILES: c1ccc(-c2ccccc2-c2ccc(-c3ccc(N(c4ccc(C5CC6CCC5C6)cc4)c4cc(-c5ccccc5)c5c(c4)C(c4ccccc4)(c4ccccc4)c4ccccc4-5)cc3)cc2)cc1]

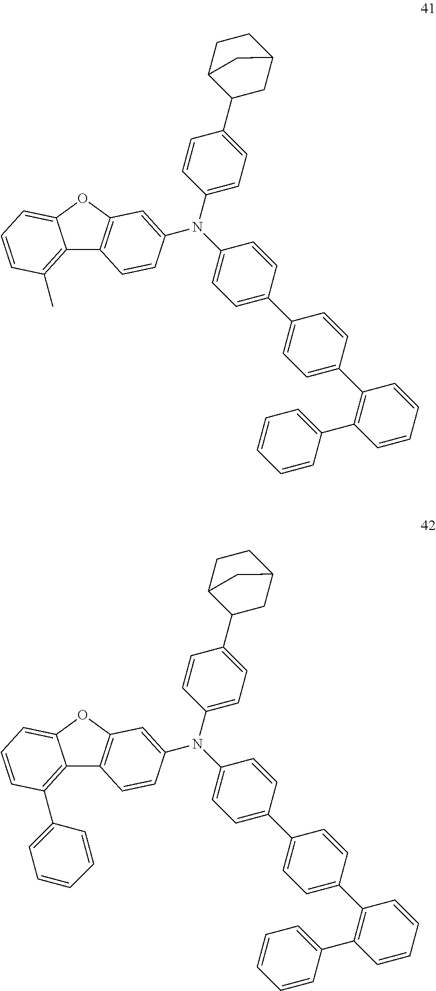 [SMILES: Cc1cccc2oc3cc(N(c4ccc(-c5ccc(-c6ccccc6-c6ccccc6)cc5)cc4)c4ccc(C5CC6CCC5C6)cc4)ccc3c12 ; c1ccc(-c2ccccc2-c2ccc(-c3ccc(N(c4ccc(C5CC6CCC5C6)cc4)c4ccc5c(c4)oc4cccc(-c6ccccc6)c45)cc3)cc2)cc1]

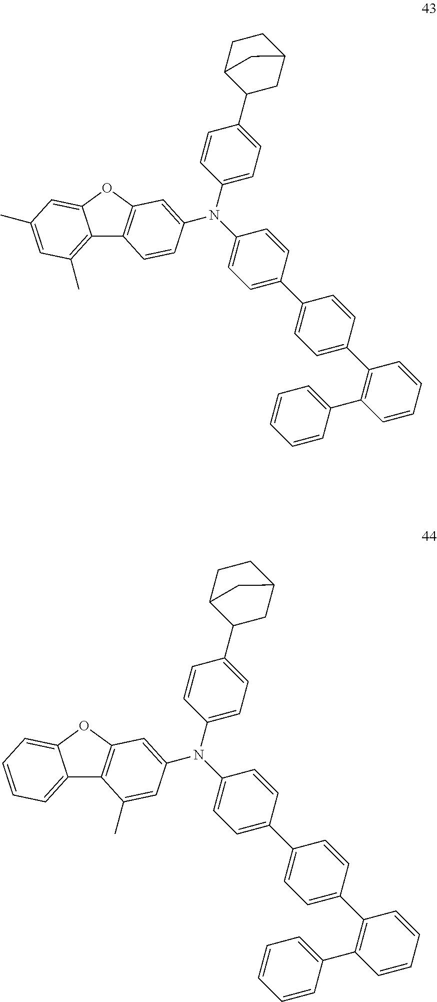 [SMILES: Cc1cc(C)c2c(c1)oc1cc(N(c3ccc(-c4ccc(-c5ccccc5-c5ccccc5)cc4)cc3)c3ccc(C4CC5CCC4C5)cc3)ccc12 ; Cc1cc(N(c2ccc(-c3ccc(-c4ccccc4-c4ccccc4)cc3)cc2)c2ccc(C3CC4CCC3C4)cc2)cc2oc3ccccc3c12]

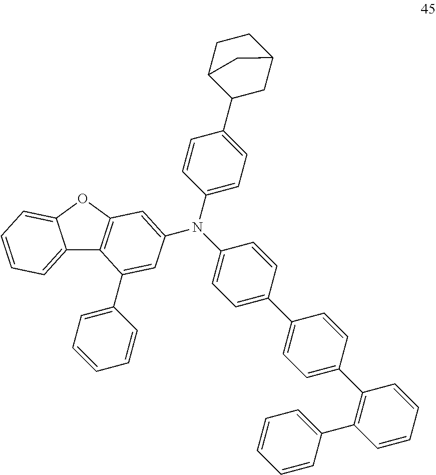 [SMILES: c1ccc(-c2ccccc2-c2ccc(-c3ccc(N(c4ccc(C5CC6CCC5C6)cc4)c4cc(-c5ccccc5)c5c(c4)oc4ccccc45)cc3)cc2)cc1]

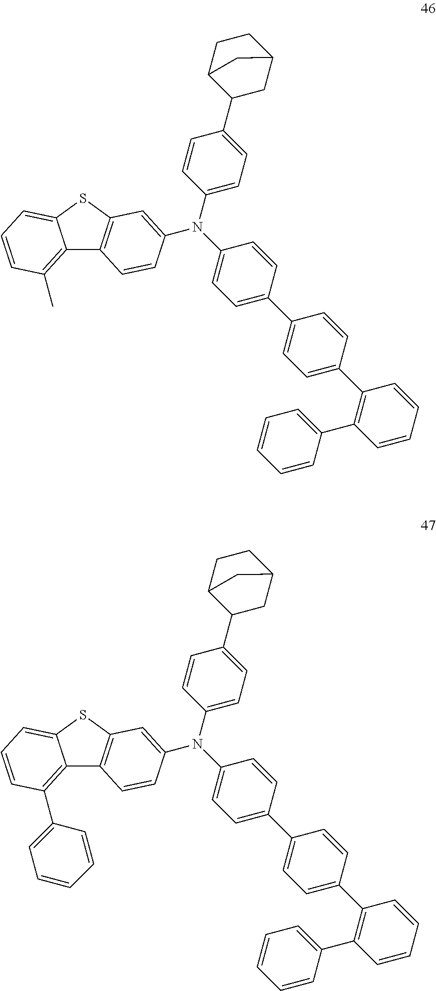 [SMILES: Cc1cccc2sc3cc(N(c4ccc(-c5ccc(-c6ccccc6-c6ccccc6)cc5)cc4)c4ccc(C5CC6CCC5C6)cc4)ccc3c12 ; c1ccc(-c2ccccc2-c2ccc(-c3ccc(N(c4ccc(C5CC6CCC5C6)cc4)c4ccc5c(c4)sc4cccc(-c6ccccc6)c45)cc3)cc2)cc1]

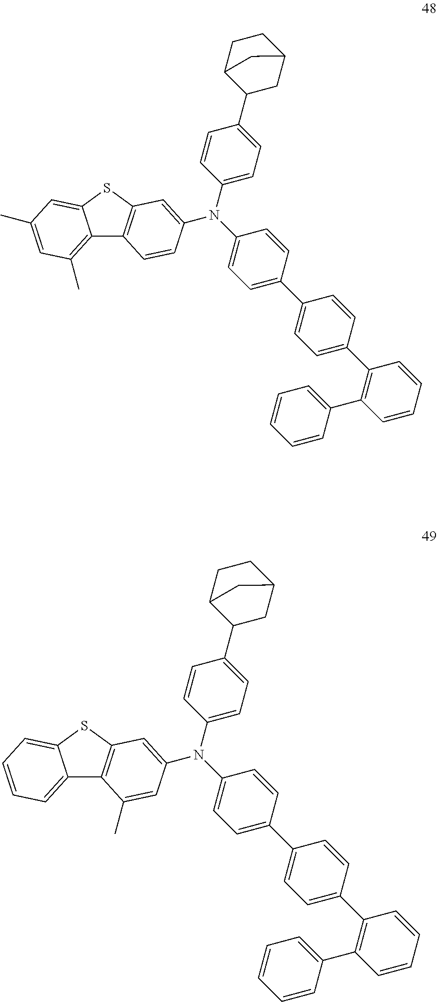 [SMILES: Cc1cc(C)c2c(c1)sc1cc(N(c3ccc(-c4ccc(-c5ccccc5-c5ccccc5)cc4)cc3)c3ccc(C4CC5CCC4C5)cc3)ccc12 ; Cc1cc(N(c2ccc(-c3ccc(-c4ccccc4-c4ccccc4)cc3)cc2)c2ccc(C3CC4CCC3C4)cc2)cc2sc3ccccc3c12]

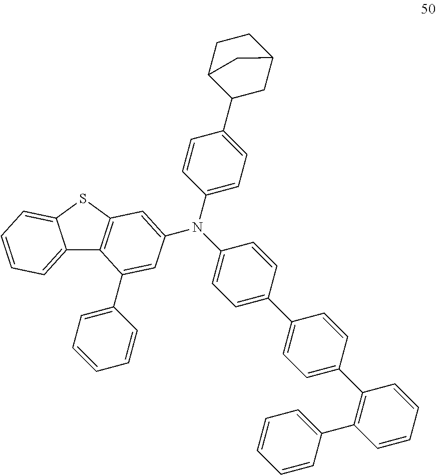 [SMILES: c1ccc(-c2ccccc2-c2ccc(-c3ccc(N(c4ccc(C5CC6CCC5C6)cc4)c4cc(-c5ccccc5)c5c(c4)sc4ccccc45)cc3)cc2)cc1]

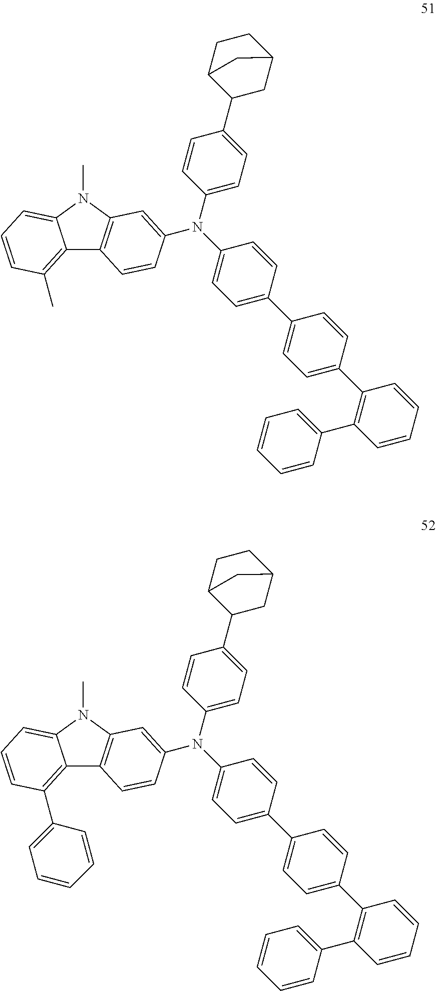 [SMILES: Cc1cccc2c1c1ccc(N(c3ccc(-c4ccc(-c5ccccc5-c5ccccc5)cc4)cc3)c3ccc(C4CC5CCC4C5)cc3)cc1n2C ; Cn1c2cc(N(c3ccc(-c4ccc(-c5ccccc5-c5ccccc5)cc4)cc3)c3ccc(C4CC5CCC4C5)cc3)ccc2c2c(-c3ccccc3)cccc21]

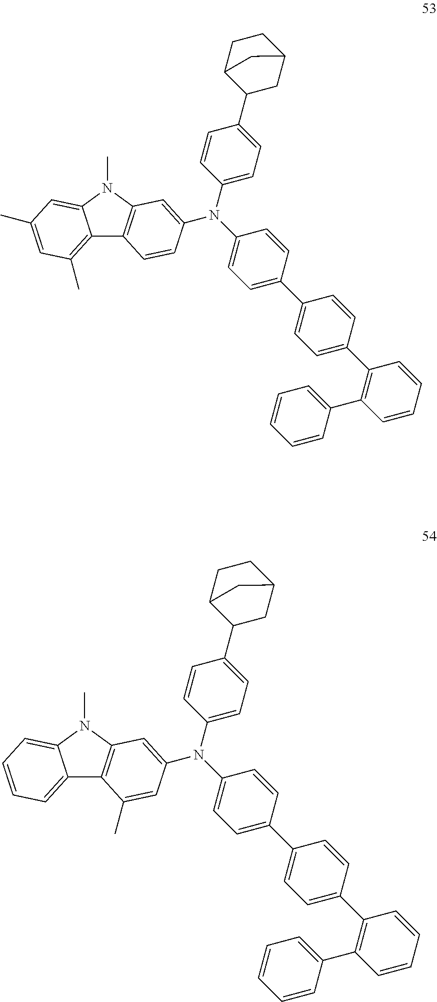 [SMILES: Cc1cc(C)c2c3ccc(N(c4ccc(-c5ccc(-c6ccccc6-c6ccccc6)cc5)cc4)c4ccc(C5CC6CCC5C6)cc4)cc3n(C)c2c1 ; Cc1cc(N(c2ccc(-c3ccc(-c4ccccc4-c4ccccc4)cc3)cc2)c2ccc(C3CC4CCC3C4)cc2)cc2c1c1ccccc1n2C]

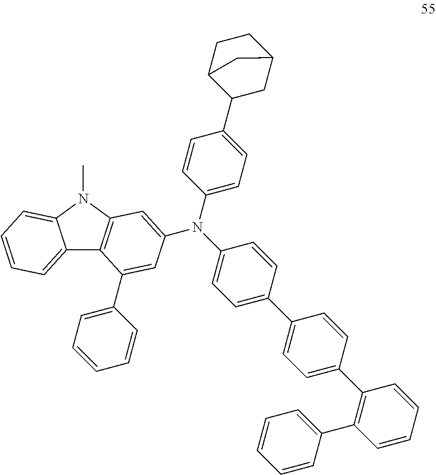 [SMILES: Cn1c2ccccc2c2c(-c3ccccc3)cc(N(c3ccc(-c4ccc(-c5ccccc5-c5ccccc5)cc4)cc3)c3ccc(C4CC5CCC4C5)cc3)cc21]

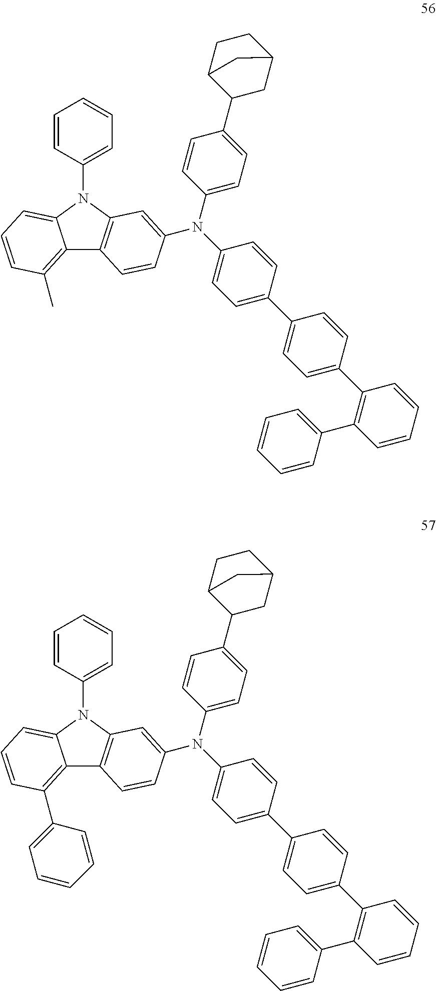 [SMILES: Cc1cccc2c1c1ccc(N(c3ccc(-c4ccc(-c5ccccc5-c5ccccc5)cc4)cc3)c3ccc(C4CC5CCC4C5)cc3)cc1n2-c1ccccc1 ; c1ccc(-c2ccccc2-c2ccc(-c3ccc(N(c4ccc(C5CC6CCC5C6)cc4)c4ccc5c6c(-c7ccccc7)cccc6n(-c6ccccc6)c5c4)cc3)cc2)cc1]

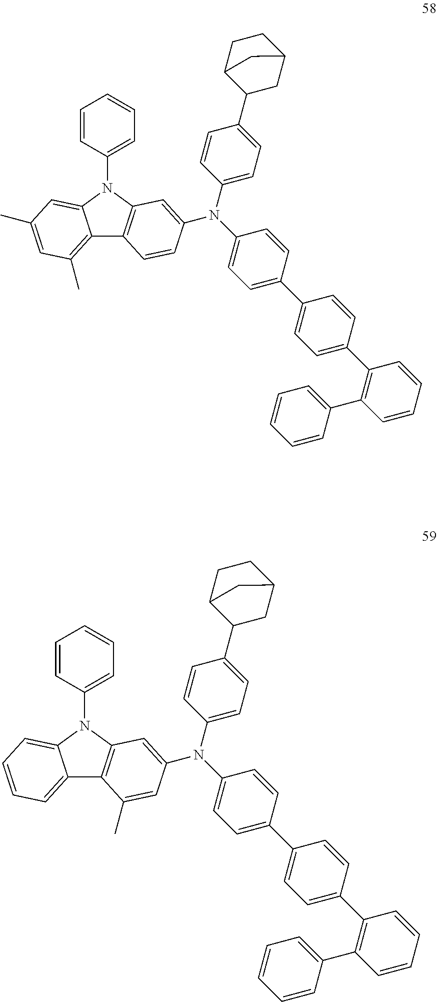 [SMILES: Cc1cc(C)c2c3ccc(N(c4ccc(-c5ccc(-c6ccccc6-c6ccccc6)cc5)cc4)c4ccc(C5CC6CCC5C6)cc4)cc3n(-c3ccccc3)c2c1 ; Cc1cc(N(c2ccc(-c3ccc(-c4ccccc4-c4ccccc4)cc3)cc2)c2ccc(C3CC4CCC3C4)cc2)cc2c1c1ccccc1n2-c1ccccc1]

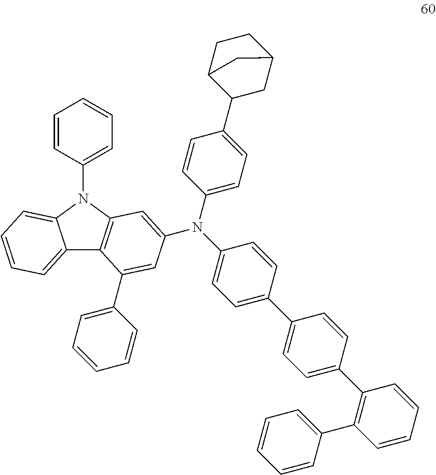 [SMILES: c1ccc(-c2ccccc2-c2ccc(-c3ccc(N(c4ccc(C5CC6CCC5C6)cc4)c4cc(-c5ccccc5)c5c6ccccc6n(-c6ccccc6)c5c4)cc3)cc2)cc1]

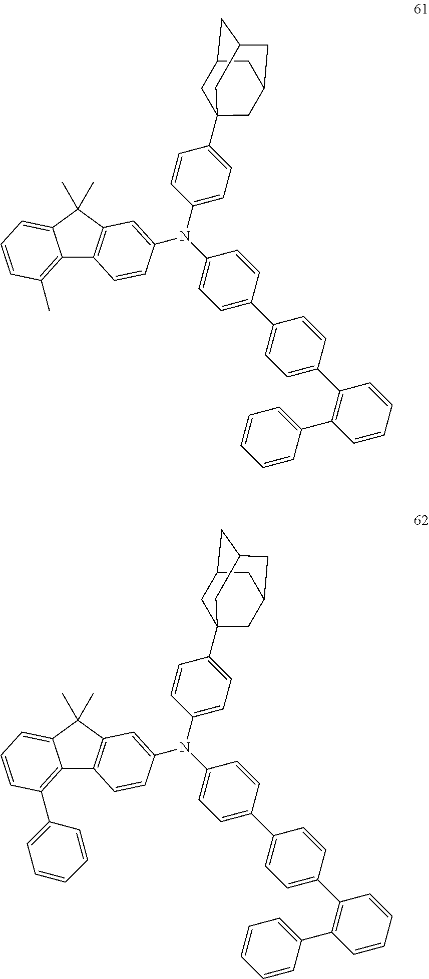 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(C)C ; CC1(C)c2cc(N(c3ccc(-c4ccc(-c5ccccc5-c5ccccc5)cc4)cc3)c3ccc(C45CC6CC(CC(C6)C4)C5)cc3)ccc2-c2c(-c3ccccc3)cccc21]

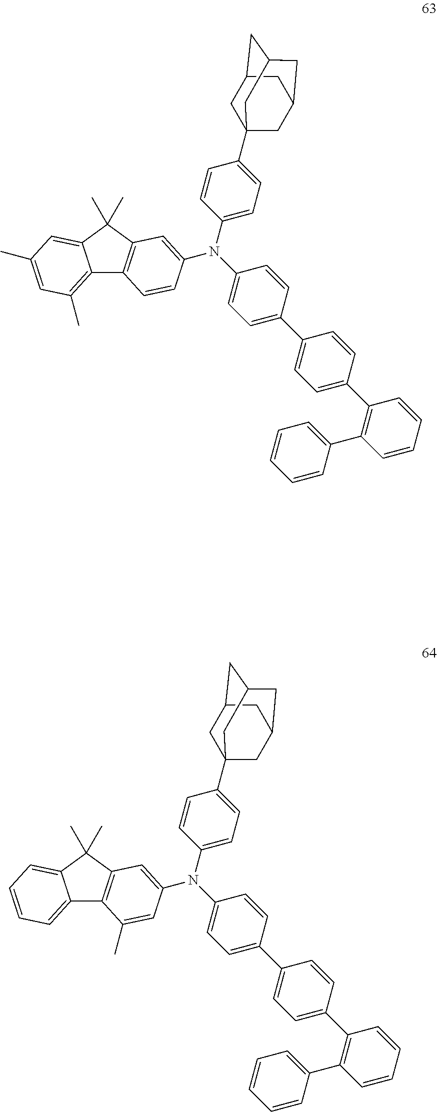 [SMILES: Cc1cc(C)c2c(c1)C(C)(C)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(C)C]

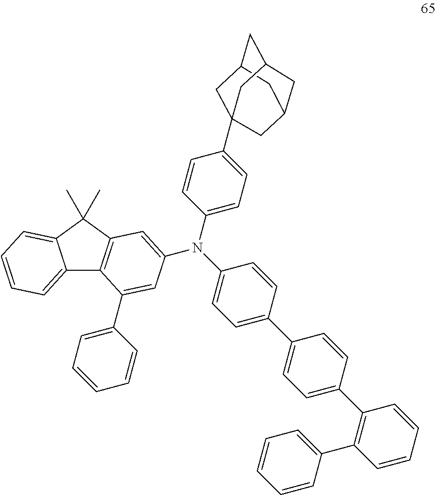 [SMILES: CC1(C)c2ccccc2-c2c(-c3ccccc3)cc(N(c3ccc(-c4ccc(-c5ccccc5-c5ccccc5)cc4)cc3)c3ccc(C45CC6CC(CC(C6)C4)C5)cc3)cc21]

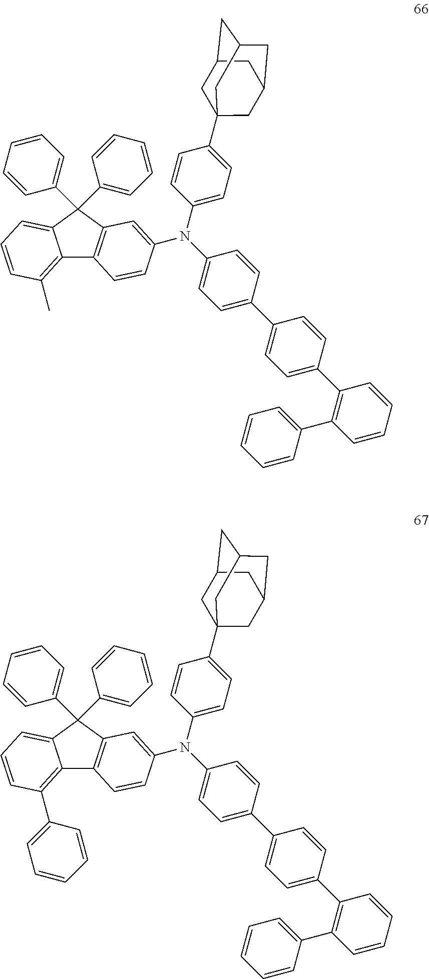 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(c1ccccc1)c1ccccc1 ; c1ccc(-c2ccccc2-c2ccc(-c3ccc(N(c4ccc(C56CC7CC(CC(C7)C5)C6)cc4)c4ccc5c(c4)C(c4ccccc4)(c4ccccc4)c4cccc(-c6ccccc6)c4-5)cc3)cc2)cc1]

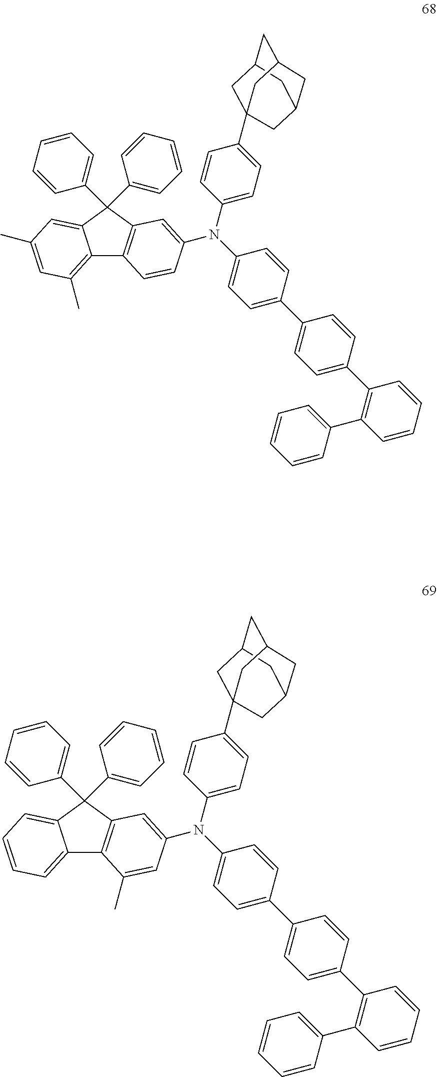 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(c1ccccc1)c1ccccc1]

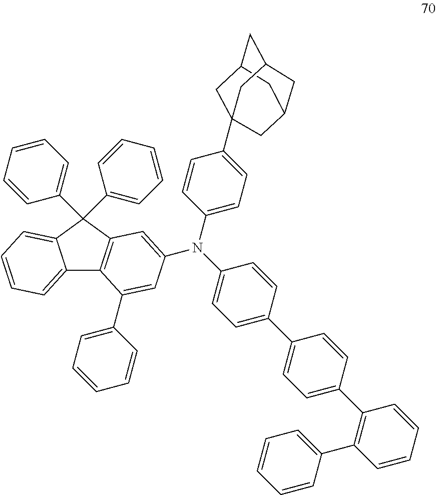 [SMILES: c1ccc(-c2ccccc2-c2ccc(-c3ccc(N(c4ccc(C56CC7CC(CC(C7)C5)C6)cc4)c4cc(-c5ccccc5)c5c(c4)C(c4ccccc4)(c4ccccc4)c4ccccc4-5)cc3)cc2)cc1]

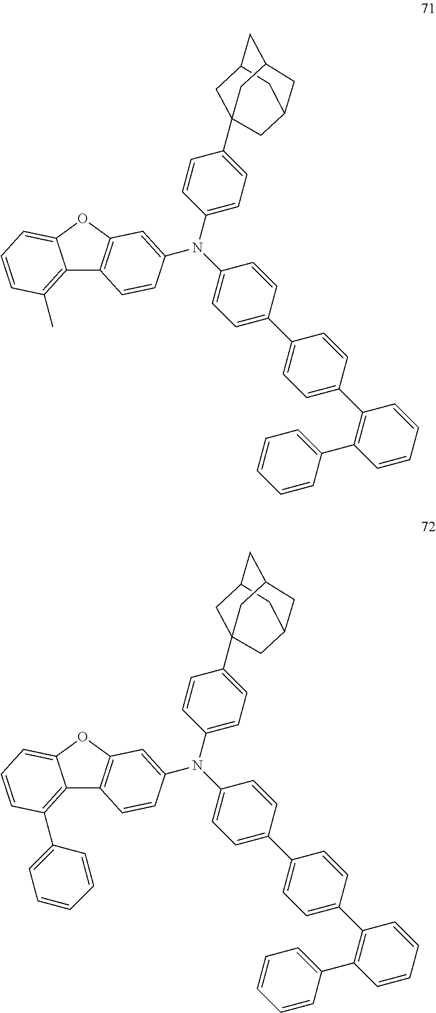 [SMILES: Cc1cccc2oc3cc(N(c4ccc(-c5ccc(-c6ccccc6-c6ccccc6)cc5)cc4)c4ccc(C56CC7CC(CC(C7)C5)C6)cc4)ccc3c12 ; c1ccc(-c2ccccc2-c2ccc(-c3ccc(N(c4ccc(C56CC7CC(CC(C7)C5)C6)cc4)c4ccc5c(c4)oc4cccc(-c6ccccc6)c45)cc3)cc2)cc1]

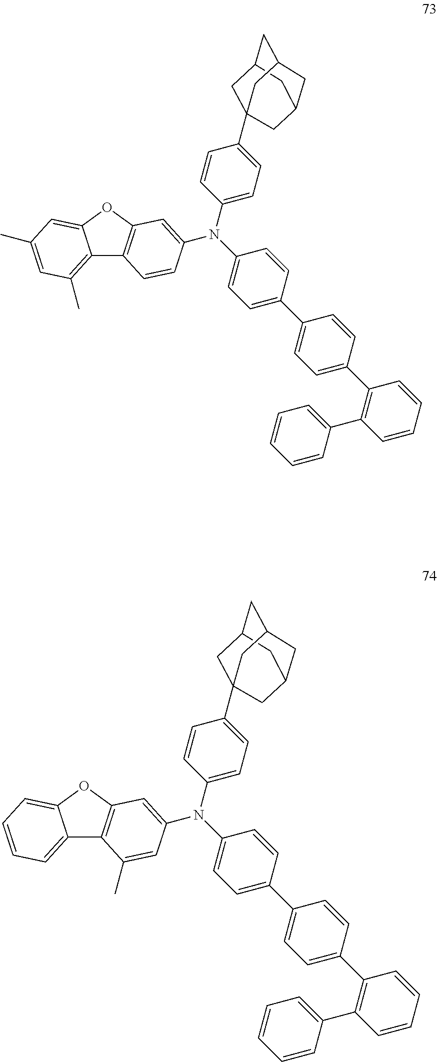 [SMILES: Cc1cc(C)c2c(c1)oc1cc(N(c3ccc(-c4ccc(-c5ccccc5-c5ccccc5)cc4)cc3)c3ccc(C45CC6CC(CC(C6)C4)C5)cc3)ccc12 ; Cc1cc(N(c2ccc(-c3ccc(-c4ccccc4-c4ccccc4)cc3)cc2)c2ccc(C34CC5CC(CC(C5)C3)C4)cc2)cc2oc3ccccc3c12]

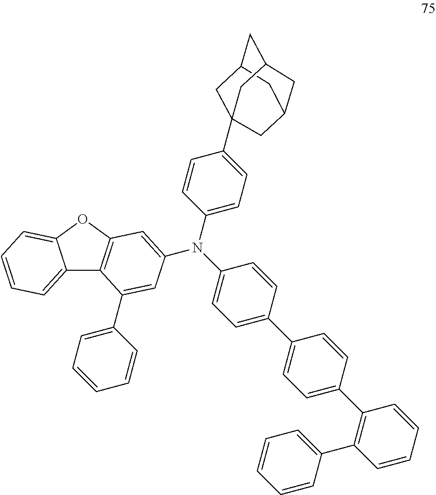 [SMILES: c1ccc(-c2ccccc2-c2ccc(-c3ccc(N(c4ccc(C56CC7CC(CC(C7)C5)C6)cc4)c4cc(-c5ccccc5)c5c(c4)oc4ccccc45)cc3)cc2)cc1]

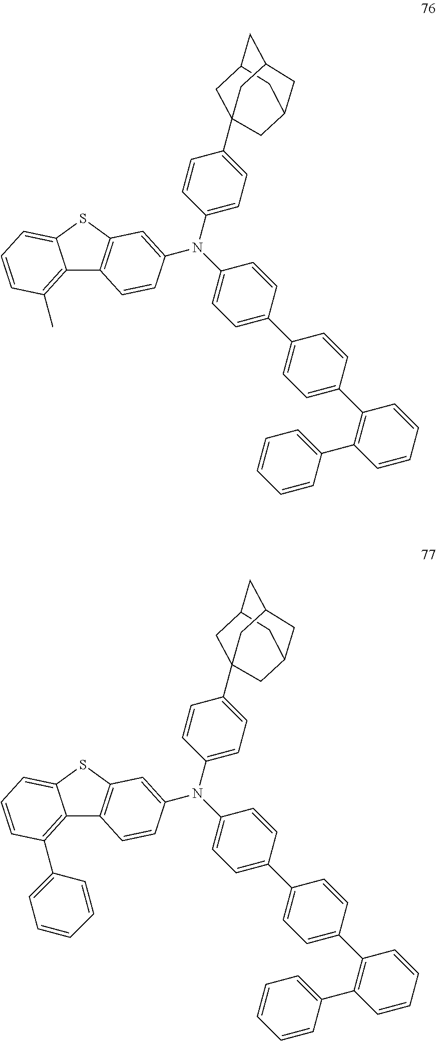 [SMILES: Cc1cccc2sc3cc(N(c4ccc(-c5ccc(-c6ccccc6-c6ccccc6)cc5)cc4)c4ccc(C56CC7CC(CC(C7)C5)C6)cc4)ccc3c12 ; c1ccc(-c2ccccc2-c2ccc(-c3ccc(N(c4ccc(C56CC7CC(CC(C7)C5)C6)cc4)c4ccc5c(c4)sc4cccc(-c6ccccc6)c45)cc3)cc2)cc1]

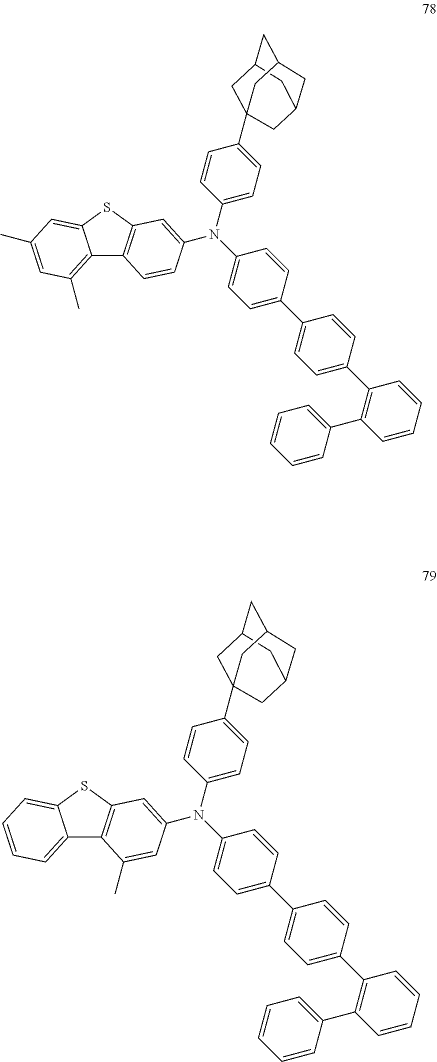 [SMILES: Cc1cc(C)c2c(c1)sc1cc(N(c3ccc(-c4ccc(-c5ccccc5-c5ccccc5)cc4)cc3)c3ccc(C45CC6CC(CC(C6)C4)C5)cc3)ccc12 ; Cc1cc(N(c2ccc(-c3ccc(-c4ccccc4-c4ccccc4)cc3)cc2)c2ccc(C34CC5CC(CC(C5)C3)C4)cc2)cc2sc3ccccc3c12]

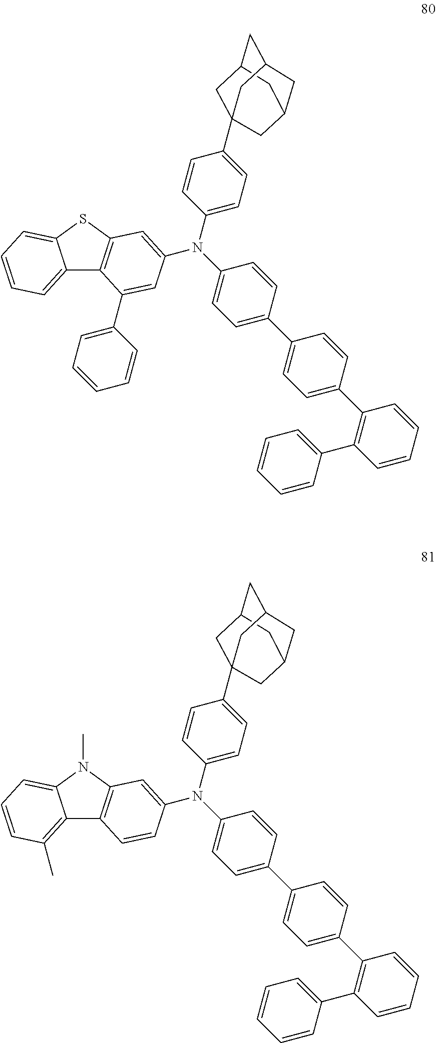 [SMILES: c1ccc(-c2ccccc2-c2ccc(-c3ccc(N(c4ccc(C56CC7CC(CC(C7)C5)C6)cc4)c4cc(-c5ccccc5)c5c(c4)sc4ccccc45)cc3)cc2)cc1 ; Cc1cccc2c1c1ccc(N(c3ccc(-c4ccc(-c5ccccc5-c5ccccc5)cc4)cc3)c3ccc(C45CC6CC(CC(C6)C4)C5)cc3)cc1n2C]

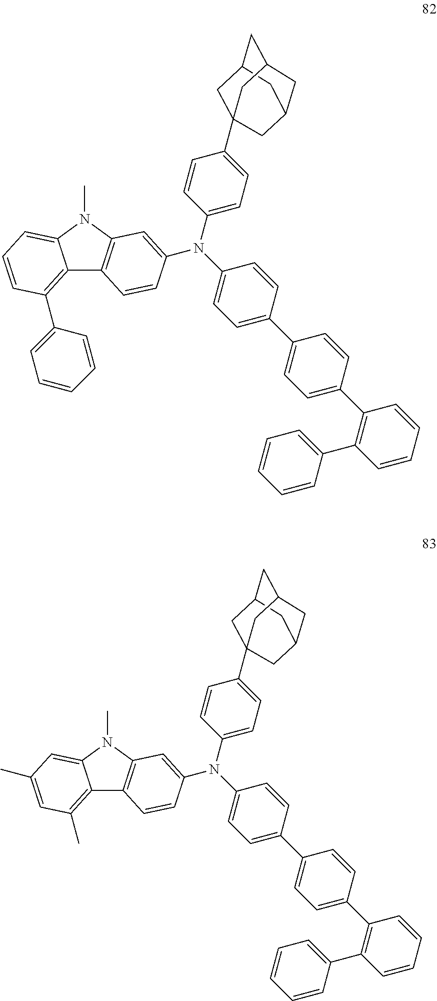 [SMILES: Cn1c2cc(N(c3ccc(-c4ccc(-c5ccccc5-c5ccccc5)cc4)cc3)c3ccc(C45CC6CC(CC(C6)C4)C5)cc3)ccc2c2c(-c3ccccc3)cccc21 ; Cc1cc(C)c2c3ccc(N(c4ccc(-c5ccc(-c6ccccc6-c6ccccc6)cc5)cc4)c4ccc(C56CC7CC(CC(C7)C5)C6)cc4)cc3n(C)c2c1]

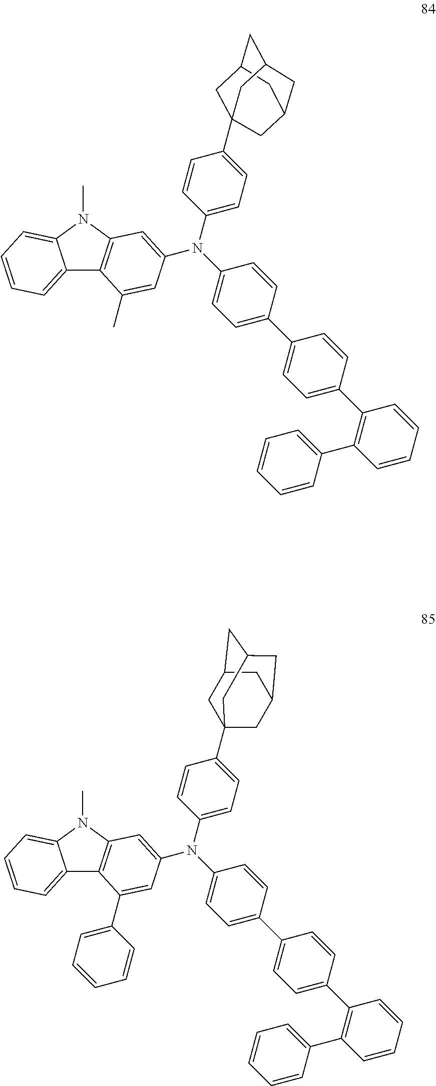 [SMILES: Cc1cc(N(c2ccc(-c3ccc(-c4ccccc4-c4ccccc4)cc3)cc2)c2ccc(C34CC5CC(CC(C5)C3)C4)cc2)cc2c1c1ccccc1n2C ; Cn1c2ccccc2c2c(-c3ccccc3)cc(N(c3ccc(-c4ccc(-c5ccccc5-c5ccccc5)cc4)cc3)c3ccc(C45CC6CC(CC(C6)C4)C5)cc3)cc21]

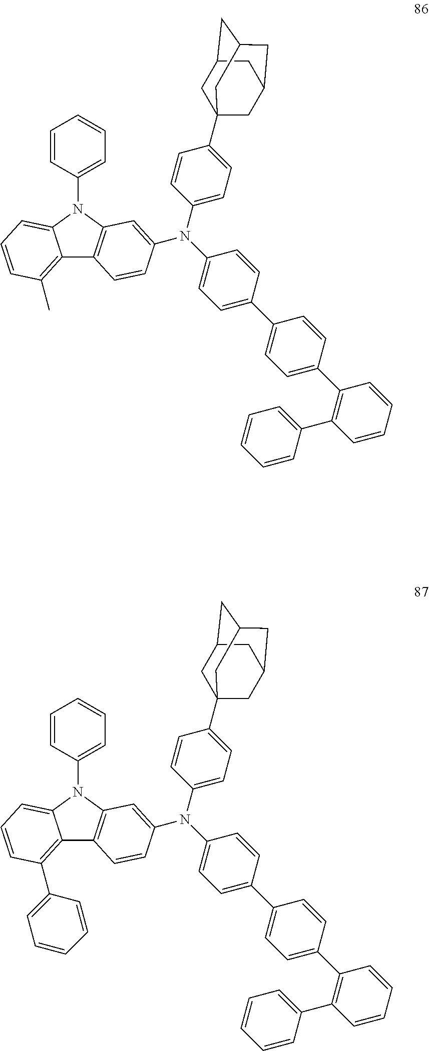 [SMILES: Cc1cccc2c1c1ccc(N(c3ccc(-c4ccc(-c5ccccc5-c5ccccc5)cc4)cc3)c3ccc(C45CC6CC(CC(C6)C4)C5)cc3)cc1n2-c1ccccc1 ; c1ccc(-c2ccccc2-c2ccc(-c3ccc(N(c4ccc(C56CC7CC(CC(C7)C5)C6)cc4)c4ccc5c6c(-c7ccccc7)cccc6n(-c6ccccc6)c5c4)cc3)cc2)cc1]

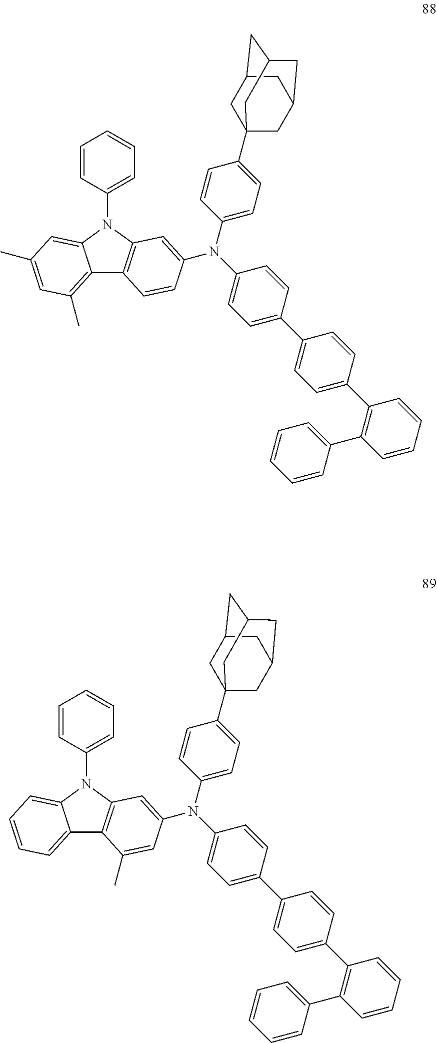 [SMILES: Cc1cc(C)c2c3ccc(N(c4ccc(-c5ccc(-c6ccccc6-c6ccccc6)cc5)cc4)c4ccc(C56CC7CC(CC(C7)C5)C6)cc4)cc3n(-c3ccccc3)c2c1 ; Cc1cc(N(c2ccc(-c3ccc(-c4ccccc4-c4ccccc4)cc3)cc2)c2ccc(C34CC5CC(CC(C5)C3)C4)cc2)cc2c1c1ccccc1n2-c1ccccc1]

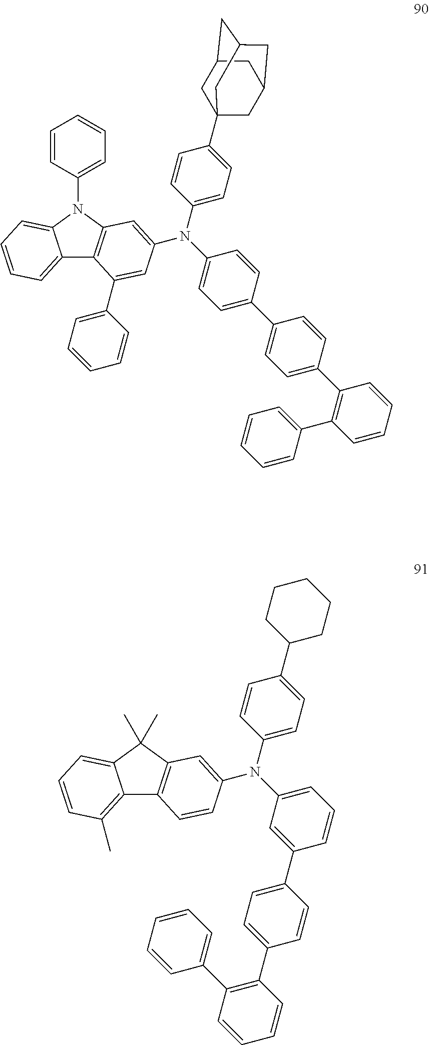 [SMILES: c1ccc(-c2ccccc2-c2ccc(-c3ccc(N(c4ccc(C56CC7CC(CC(C7)C5)C6)cc4)c4cc(-c5ccccc5)c5c6ccccc6n(-c6ccccc6)c5c4)cc3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2(C)C]

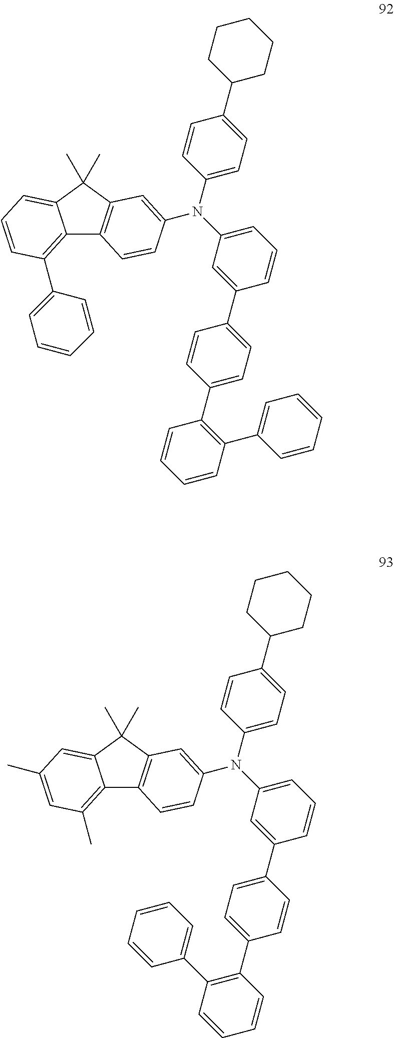 [SMILES: CC1(C)c2cc(N(c3ccc(C4CCCCC4)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C4CCCCC4)cc3)c3cccc(-c4ccc(-c5ccccc5-c5ccccc5)cc4)c3)ccc1-2]

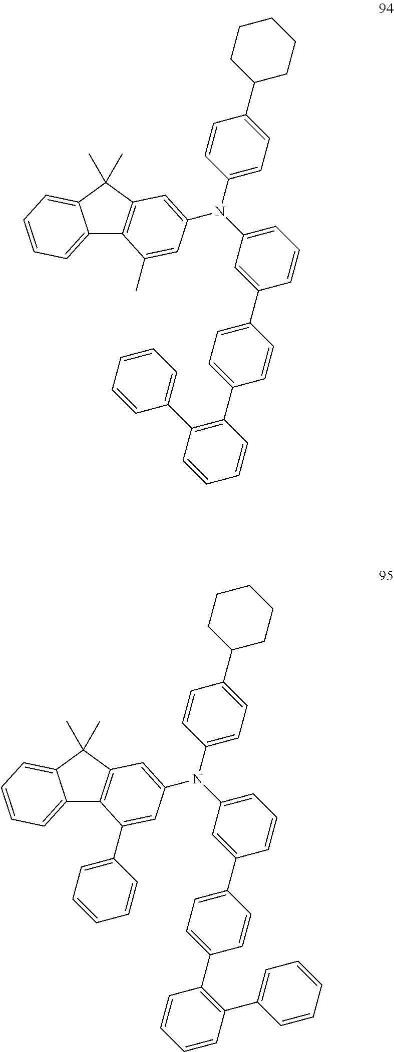 [SMILES: Cc1cc(N(c2ccc(C3CCCCC3)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CCCCC4)cc3)c3cccc(-c4ccc(-c5ccccc5-c5ccccc5)cc4)c3)cc21]

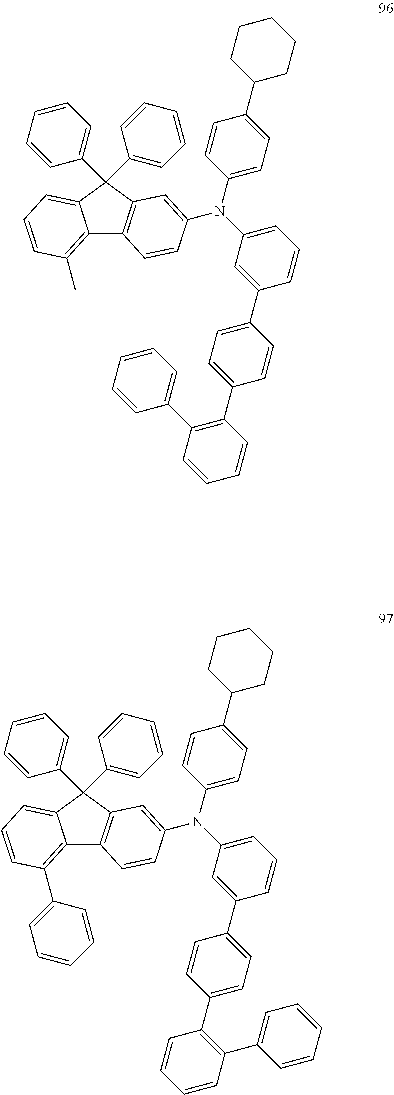 [SMILES: Cc1cccc2c1-c1ccc(N(c3ccc(C4CCCCC4)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CCCCC5)cc4)c4ccc5c(c4)C(c4ccccc4)(c4ccccc4)c4cccc(-c6ccccc6)c4-5)c3)cc2)cc1]

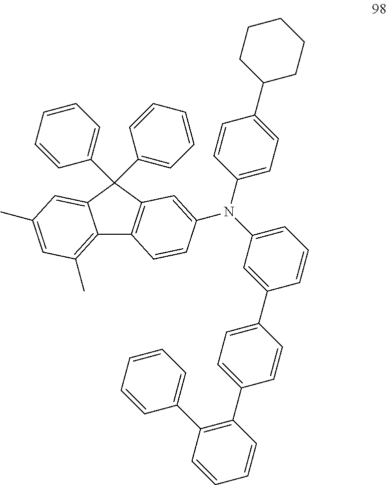 [SMILES: Cc1cc(C)c2c(c1)C(c1ccccc1)(c1ccccc1)c1cc(N(c3ccc(C4CCCCC4)cc3)c3cccc(-c4ccc(-c5ccccc5-c5ccccc5)cc4)c3)ccc1-2]

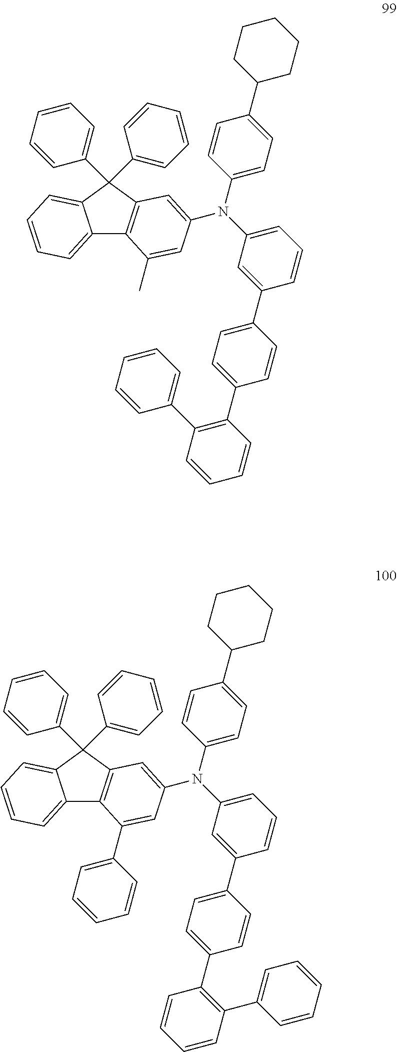 [SMILES: Cc1cc(N(c2ccc(C3CCCCC3)cc2)c2cccc(-c3ccc(-c4ccccc4-c4ccccc4)cc3)c2)cc2c1-c1ccccc1C2(c1ccccc1)c1ccccc1 ; c1ccc(-c2ccccc2-c2ccc(-c3cccc(N(c4ccc(C5CCCCC5)cc4)c4cc(-c5ccccc5)c5c(c4)C(c4ccccc4)(c4ccccc4)c4ccccc4-5)c3)cc2)cc1]

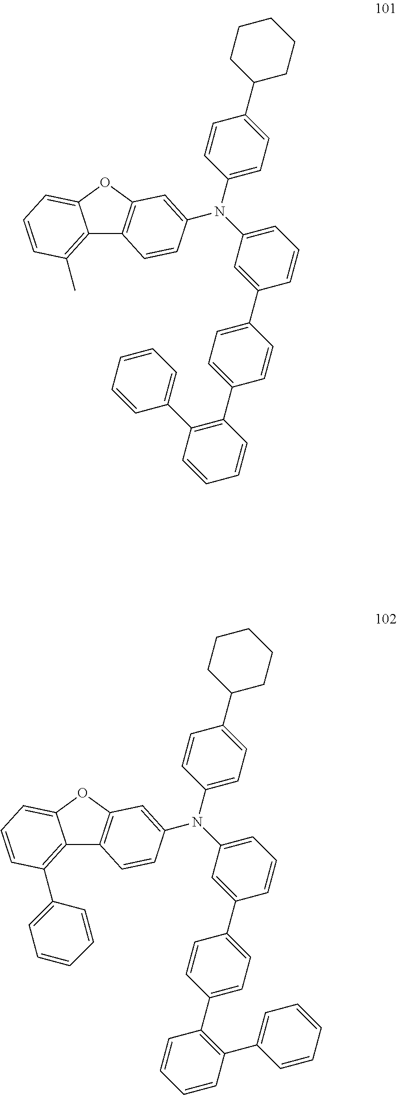 [SMILES: Cc1cccc2oc3cc(N(c4ccc(C5CCCCC5)cc4)c4cccc(-c5ccc(-c6ccccc6-c6ccccc6)cc5)c4)ccc3c12 ; c1ccc(-c2ccccc2-c2ccc(-c3cccc(N(c4ccc(C5CCCCC5)cc4)c4ccc5c(c4)oc4cccc(-c6ccccc6)c45)c3)cc2)cc1]

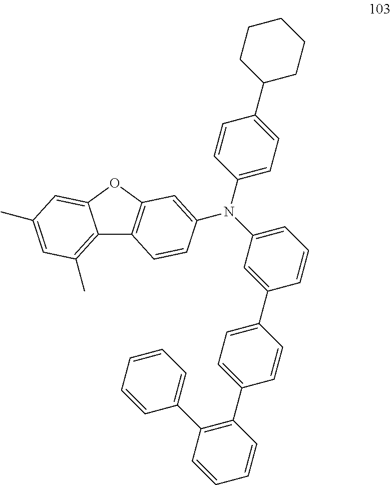 [SMILES: Cc1cc(C)c2c(c1)oc1cc(N(c3ccc(C4CCCCC4)cc3)c3cccc(-c4ccc(-c5ccccc5-c5ccccc5)cc4)c3)ccc12]

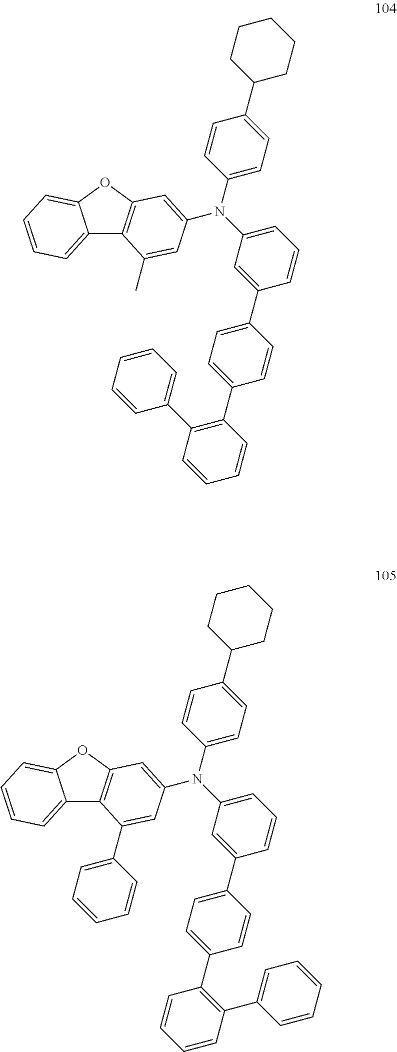 [SMILES: Cc1cc(N(c2ccc(C3CCCCC3)cc2)c2cccc(-c3ccc(-c4ccccc4-c4ccccc4)cc3)c2)cc2oc3ccccc3c12 ; c1ccc(-c2ccccc2-c2ccc(-c3cccc(N(c4ccc(C5CCCCC5)cc4)c4cc(-c5ccccc5)c5c(c4)oc4ccccc45)c3)cc2)cc1]

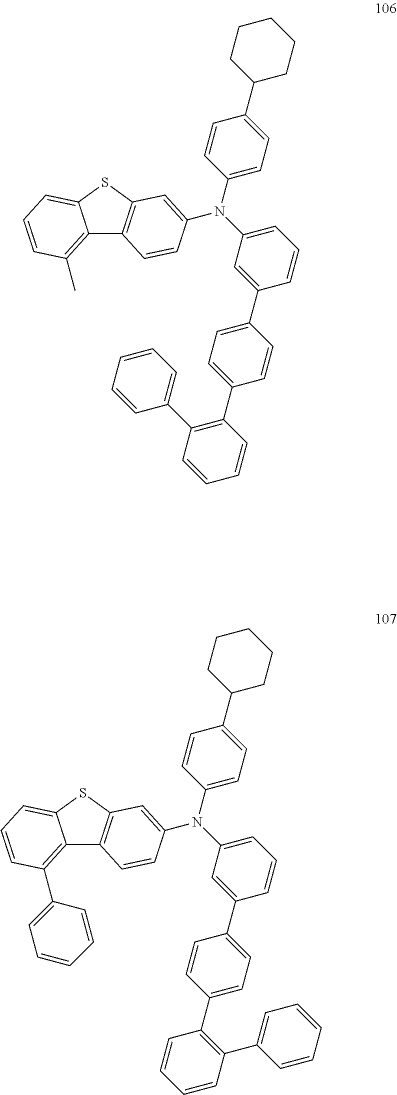 [SMILES: Cc1cccc2sc3cc(N(c4ccc(C5CCCCC5)cc4)c4cccc(-c5ccc(-c6ccccc6-c6ccccc6)cc5)c4)ccc3c12 ; c1ccc(-c2ccccc2-c2ccc(-c3cccc(N(c4ccc(C5CCCCC5)cc4)c4ccc5c(c4)sc4cccc(-c6ccccc6)c45)c3)cc2)cc1]

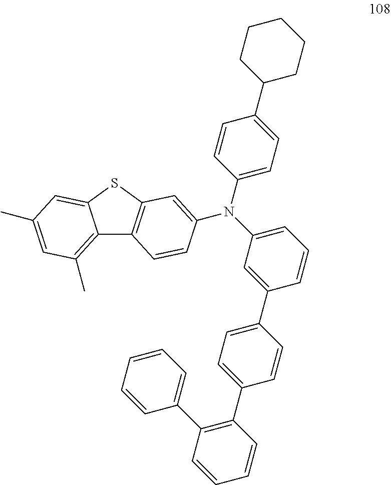 [SMILES: Cc1cc(C)c2c(c1)sc1cc(N(c3ccc(C4CCCCC4)cc3)c3cccc(-c4ccc(-c5ccccc5-c5ccccc5)cc4)c3)ccc12]

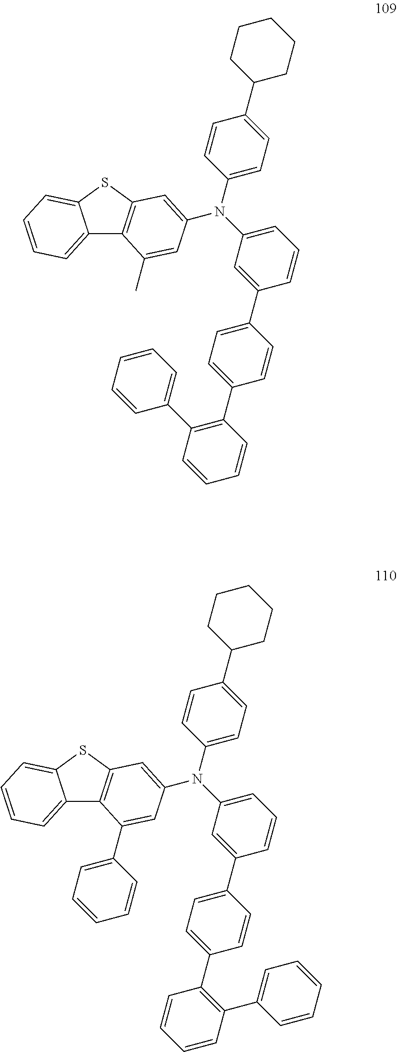 [SMILES: Cc1cc(N(c2ccc(C3CCCCC3)cc2)c2cccc(-c3ccc(-c4ccccc4-c4ccccc4)cc3)c2)cc2sc3ccccc3c12 ; c1ccc(-c2ccccc2-c2ccc(-c3cccc(N(c4ccc(C5CCCCC5)cc4)c4cc(-c5ccccc5)c5c(c4)sc4ccccc45)c3)cc2)cc1]

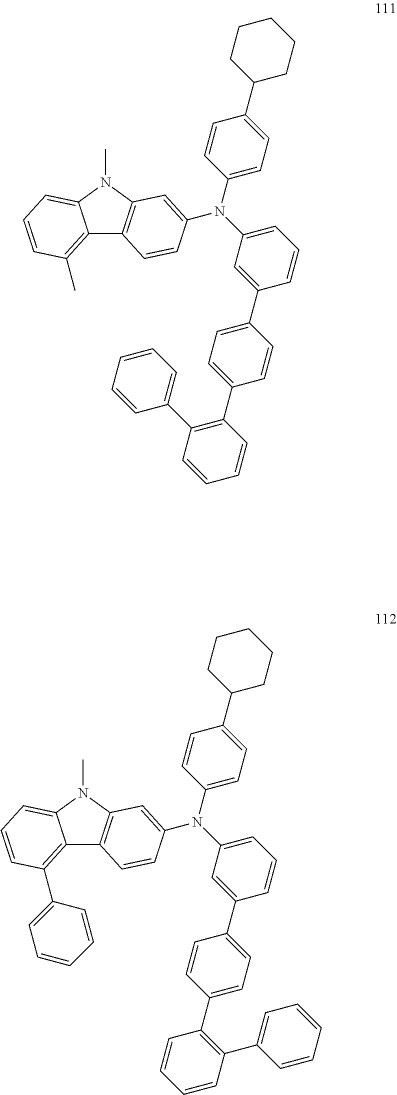 [SMILES: Cc1cccc2c1c1ccc(N(c3ccc(C4CCCCC4)cc3)c3cccc(-c4ccc(-c5ccccc5-c5ccccc5)cc4)c3)cc1n2C ; Cn1c2cc(N(c3ccc(C4CCCCC4)cc3)c3cccc(-c4ccc(-c5ccccc5-c5ccccc5)cc4)c3)ccc2c2c(-c3ccccc3)cccc21]

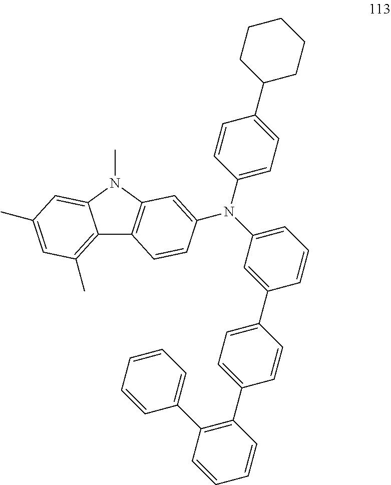 [SMILES: Cc1cc(C)c2c3ccc(N(c4ccc(C5CCCCC5)cc4)c4cccc(-c5ccc(-c6ccccc6-c6ccccc6)cc5)c4)cc3n(C)c2c1]

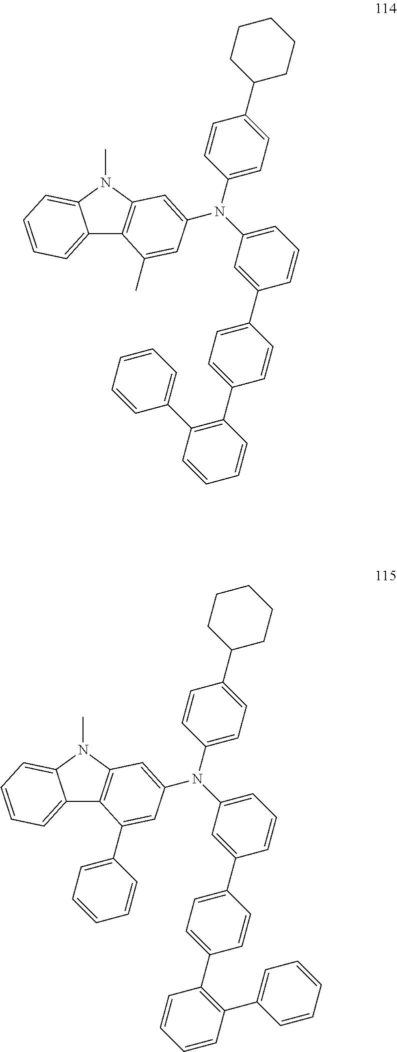 [SMILES: Cc1cc(N(c2ccc(C3CCCCC3)cc2)c2cccc(-c3ccc(-c4ccccc4-c4ccccc4)cc3)c2)cc2c1c1ccccc1n2C ; Cn1c2ccccc2c2c(-c3ccccc3)cc(N(c3ccc(C4CCCCC4)cc3)c3cccc(-c4ccc(-c5ccccc5-c5ccccc5)cc4)c3)cc21]

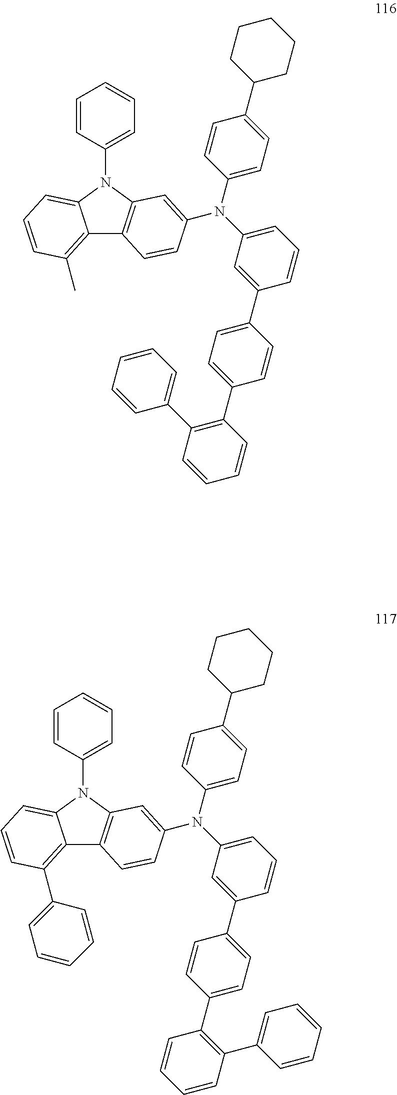 [SMILES: Cc1cccc2c1c1ccc(N(c3ccc(C4CCCCC4)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C5CCCCC5)cc4)c4ccc5c6c(-c7ccccc7)cccc6n(-c6ccccc6)c5c4)c3)cc2)cc1]

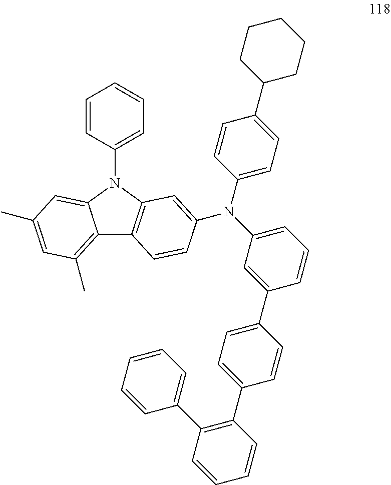 [SMILES: Cc1cc(C)c2c3ccc(N(c4ccc(C5CCCCC5)cc4)c4cccc(-c5ccc(-c6ccccc6-c6ccccc6)cc5)c4)cc3n(-c3ccccc3)c2c1]

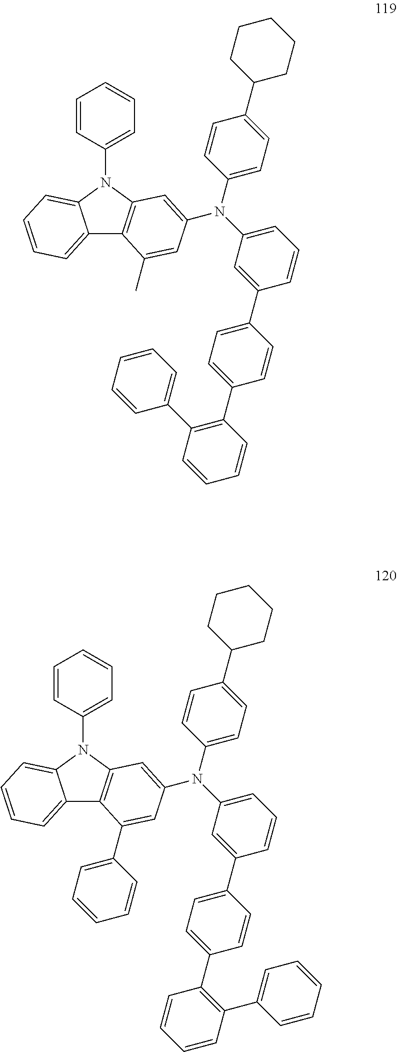 [SMILES: Cc1cc(N(c2ccc(C3CCCCC3)cc2)c2cccc(-c3ccc(-c4ccccc4-c4ccccc4)cc3)c2)cc2c1c1ccccc1n2-c1ccccc1 ; c1ccc(-c2ccccc2-c2ccc(-c3cccc(N(c4ccc(C5CCCCC5)cc4)c4cc(-c5ccccc5)c5c6ccccc6n(-c6ccccc6)c5c4)c3)cc2)cc1]

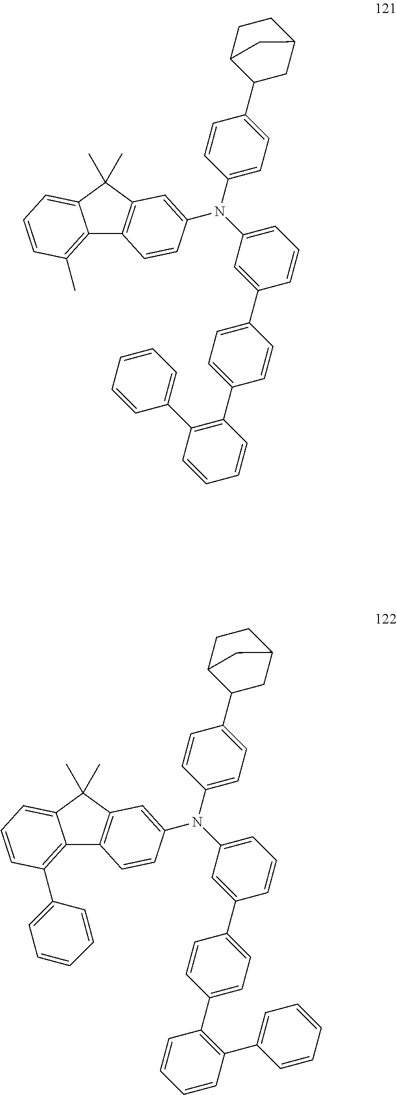 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2(C)C ; CC1(C)c2cc(N(c3ccc(C4CC5CCC4C5)cc3)c3cccc(-c4ccc(-c5ccccc5-c5ccccc5)cc4)c3)ccc2-c2c(-c3ccccc3)cccc21]

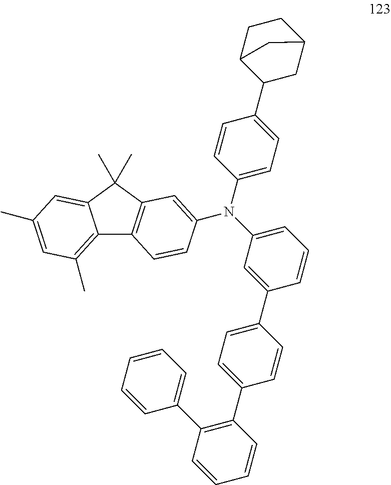 [SMILES: Cc1cc(C)c2c(c1)C(C)(C)c1cc(N(c3ccc(C4CC5CCC4C5)cc3)c3cccc(-c4ccc(-c5ccccc5-c5ccccc5)cc4)c3)ccc1-2]

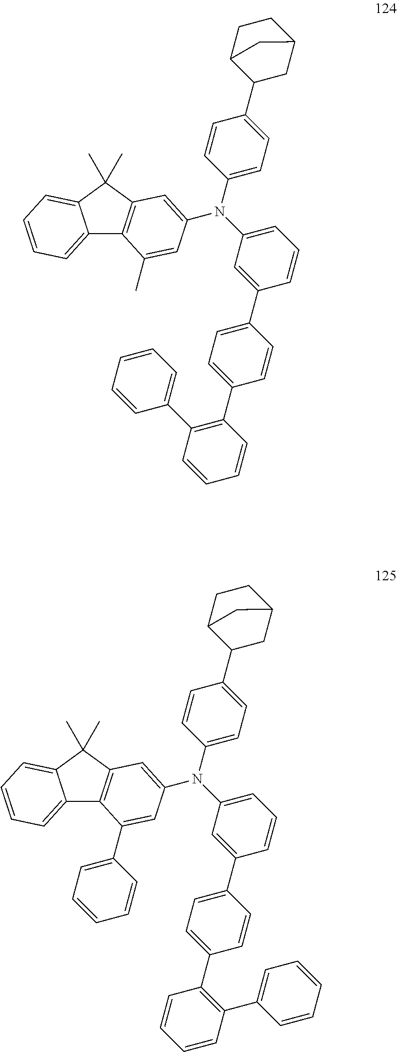 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CC5CCC4C5)cc3)c3cccc(-c4ccc(-c5ccccc5-c5ccccc5)cc4)c3)cc21]

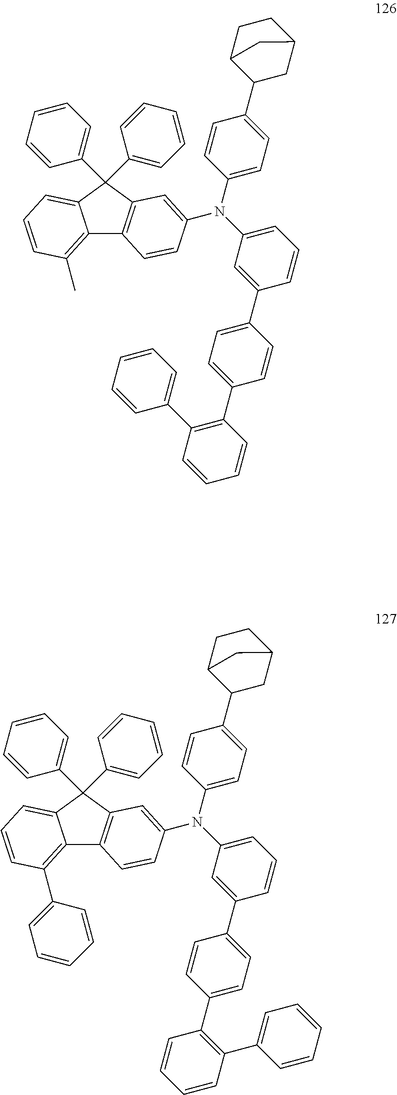 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CC6CCC5C6)cc4)c4ccc5c(c4)C(c4ccccc4)(c4ccccc4)c4cccc(-c6ccccc6)c4-5)c3)cc2)cc1]

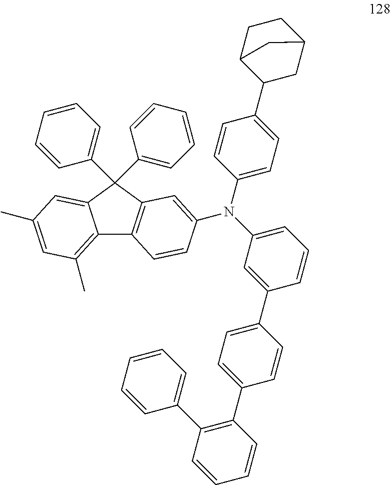 [SMILES: Cc1cc(C)c2c(c1)C(c1ccccc1)(c1ccccc1)c1cc(N(c3ccc(C4CC5CCC4C5)cc3)c3cccc(-c4ccc(-c5ccccc5-c5ccccc5)cc4)c3)ccc1-2]

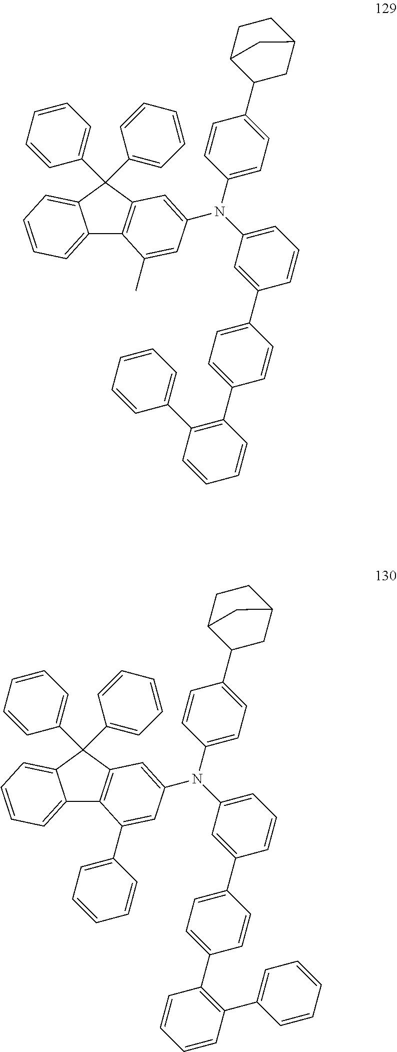 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2(c1ccccc1)c1ccccc1 ; c1ccc(-c2ccccc2-c2ccc(-c3cccc(N(c4ccc(C5CC6CCC5C6)cc4)c4cc(-c5ccccc5)c5c(c4)C(c4ccccc4)(c4ccccc4)c4ccccc4-5)c3)cc2)cc1]

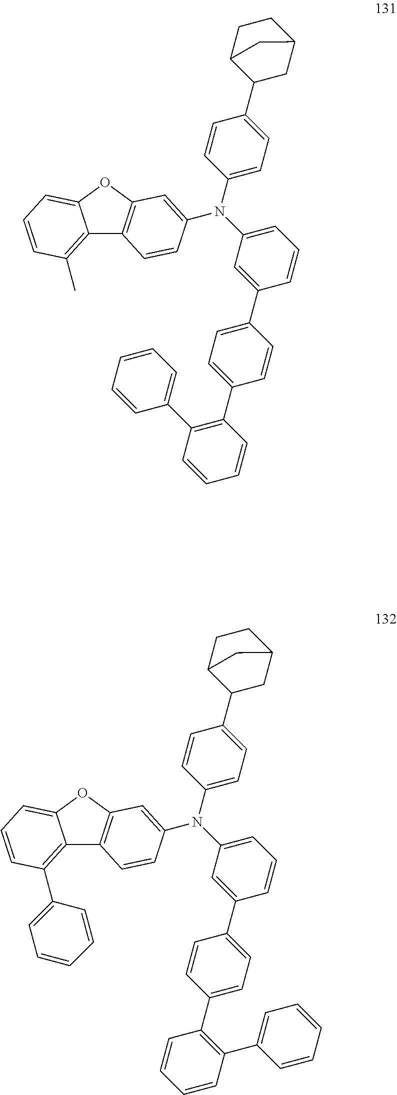 [SMILES: Cc1cccc2oc3cc(N(c4ccc(C5CC6CCC5C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C5CC6CCC5C6)cc4)c4ccc5c(c4)oc4cccc(-c6ccccc6)c45)c3)cc2)cc1]

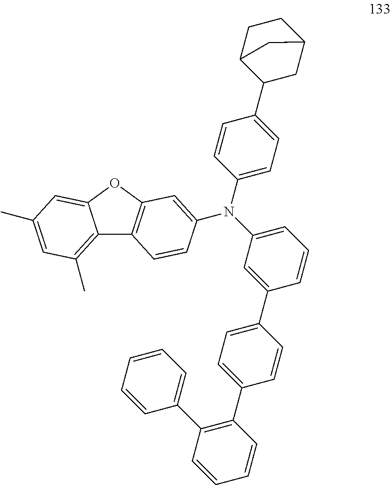 [SMILES: Cc1cc(C)c2c(c1)oc1cc(N(c3ccc(C4CC5CCC4C5)cc3)c3cccc(-c4ccc(-c5ccccc5-c5ccccc5)cc4)c3)ccc12]

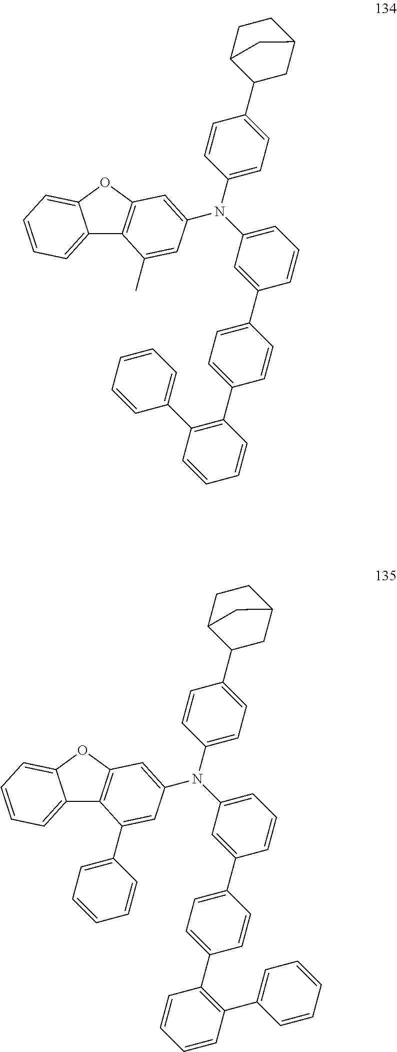 [SMILES: Cc1cc(N(c2ccc(C3CC4CCC3C4)cc2)c2cccc(-c3ccc(-c4ccccc4-c4ccccc4)cc3)c2)cc2oc3ccccc3c12 ; c1ccc(-c2ccccc2-c2ccc(-c3cccc(N(c4ccc(C5CC6CCC5C6)cc4)c4cc(-c5ccccc5)c5c(c4)oc4ccccc45)c3)cc2)cc1]

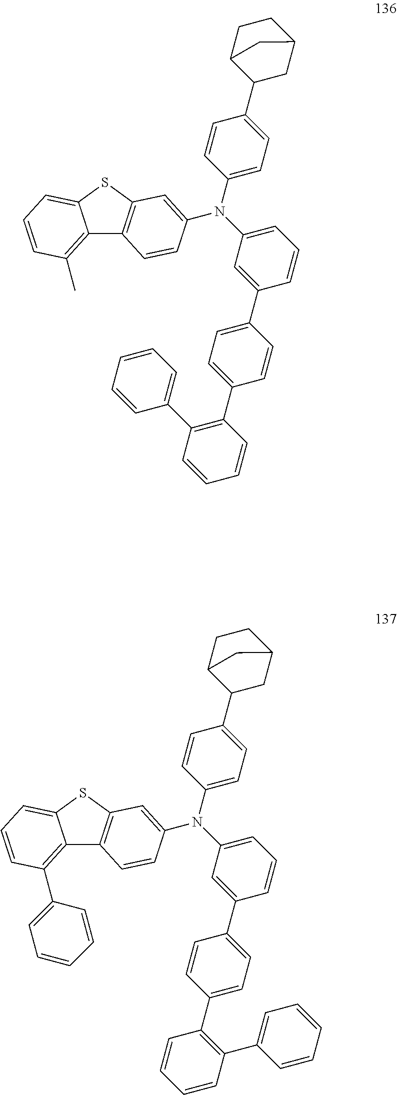 [SMILES: Cc1cccc2sc3cc(N(c4ccc(C5CC6CCC5C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C5CC6CCC5C6)cc4)c4ccc5c(c4)sc4cccc(-c6ccccc6)c45)c3)cc2)cc1]

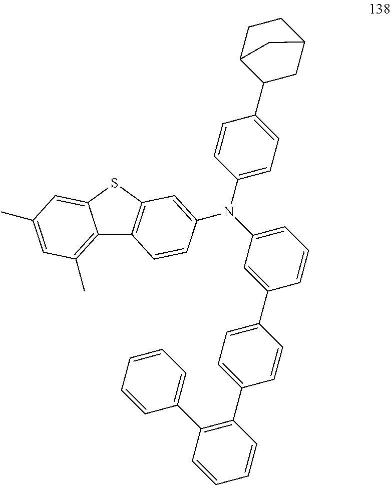 [SMILES: Cc1cc(C)c2c(c1)sc1cc(N(c3ccc(C4CC5CCC4C5)cc3)c3cccc(-c4ccc(-c5ccccc5-c5ccccc5)cc4)c3)ccc12]

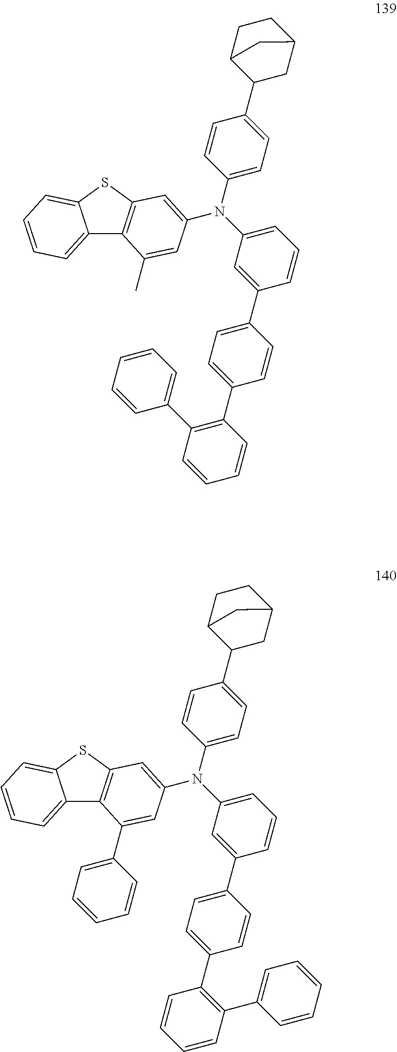 [SMILES: Cc1cc(N(c2ccc(C3CC4CCC3C4)cc2)c2cccc(-c3ccc(-c4ccccc4-c4ccccc4)cc3)c2)cc2sc3ccccc3c12 ; c1ccc(-c2ccccc2-c2ccc(-c3cccc(N(c4ccc(C5CC6CCC5C6)cc4)c4cc(-c5ccccc5)c5c(c4)sc4ccccc45)c3)cc2)cc1]

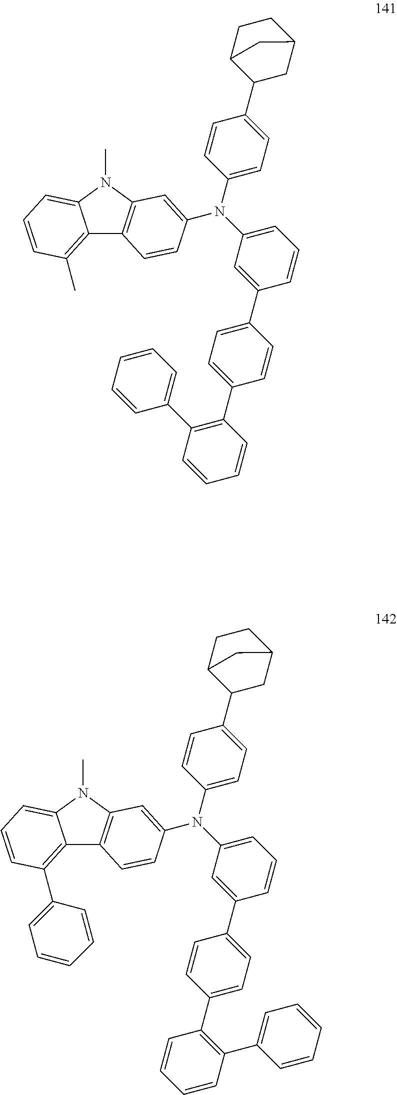 [SMILES: Cc1cccc2c1c1ccc(N(c3ccc(C4CC5CCC4C5)cc3)c3cccc(-c4ccc(-c5ccccc5-c5ccccc5)cc4)c3)cc1n2C ; Cn1c2cc(N(c3ccc(C4CC5CCC4C5)cc3)c3cccc(-c4ccc(-c5ccccc5-c5ccccc5)cc4)c3)ccc2c2c(-c3ccccc3)cccc21]

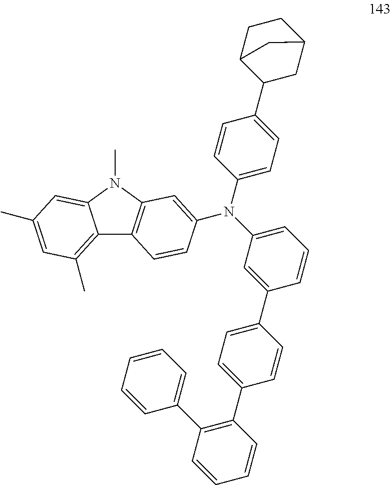 [SMILES: Cc1cc(C)c2c3ccc(N(c4ccc(C5CC6CCC5C6)cc4)c4cccc(-c5ccc(-c6ccccc6-c6ccccc6)cc5)c4)cc3n(C)c2c1]

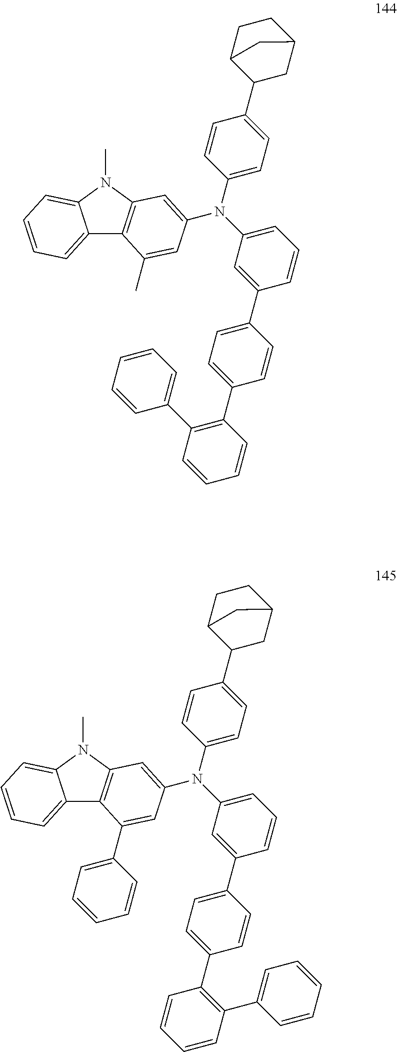 [SMILES: Cc1cc(N(c2ccc(C3CC4CCC3C4)cc2)c2cccc(-c3ccc(-c4ccccc4-c4ccccc4)cc3)c2)cc2c1c1ccccc1n2C ; Cn1c2ccccc2c2c(-c3ccccc3)cc(N(c3ccc(C4CC5CCC4C5)cc3)c3cccc(-c4ccc(-c5ccccc5-c5ccccc5)cc4)c3)cc21]

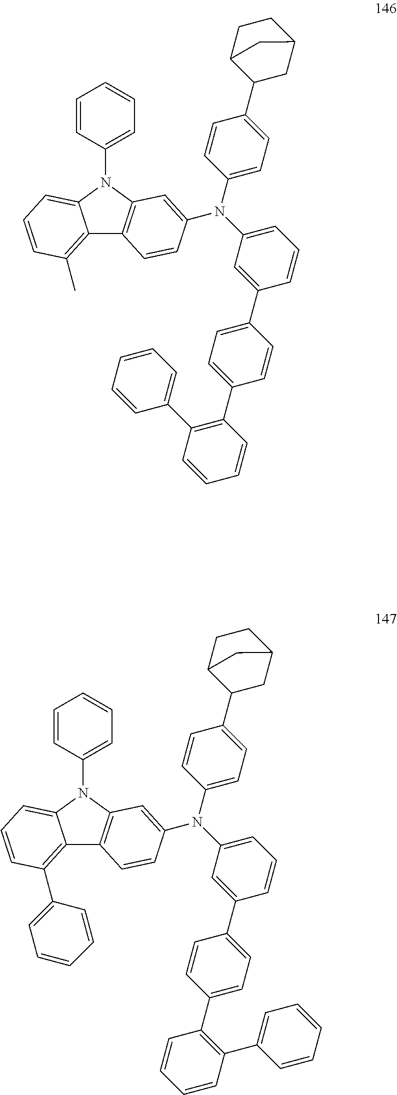 [SMILES: Cc1cccc2c1c1ccc(N(c3ccc(C4CC5CCC4C5)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C5CC6CCC5C6)cc4)c4ccc5c6c(-c7ccccc7)cccc6n(-c6ccccc6)c5c4)c3)cc2)cc1]

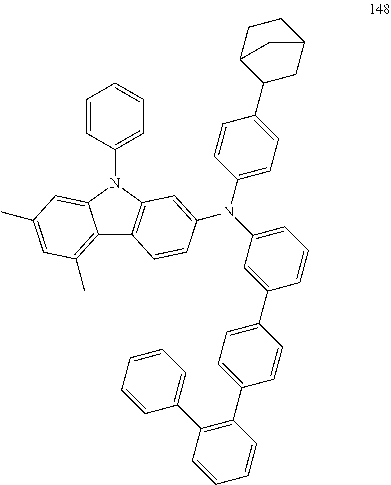 [SMILES: Cc1cc(C)c2c3ccc(N(c4ccc(C5CC6CCC5C6)cc4)c4cccc(-c5ccc(-c6ccccc6-c6ccccc6)cc5)c4)cc3n(-c3ccccc3)c2c1]

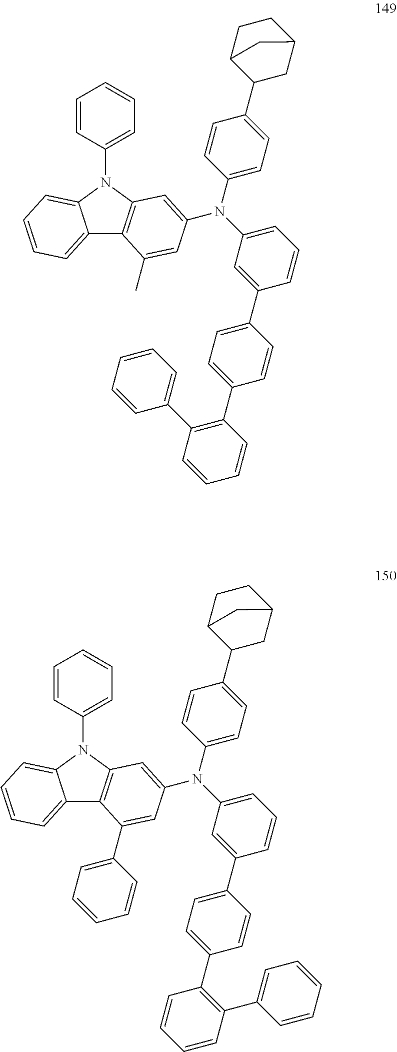 [SMILES: Cc1cc(N(c2ccc(C3CC4CCC3C4)cc2)c2cccc(-c3ccc(-c4ccccc4-c4ccccc4)cc3)c2)cc2c1c1ccccc1n2-c1ccccc1 ; c1ccc(-c2ccccc2-c2ccc(-c3cccc(N(c4ccc(C5CC6CCC5C6)cc4)c4cc(-c5ccccc5)c5c6ccccc6n(-c6ccccc6)c5c4)c3)cc2)cc1]

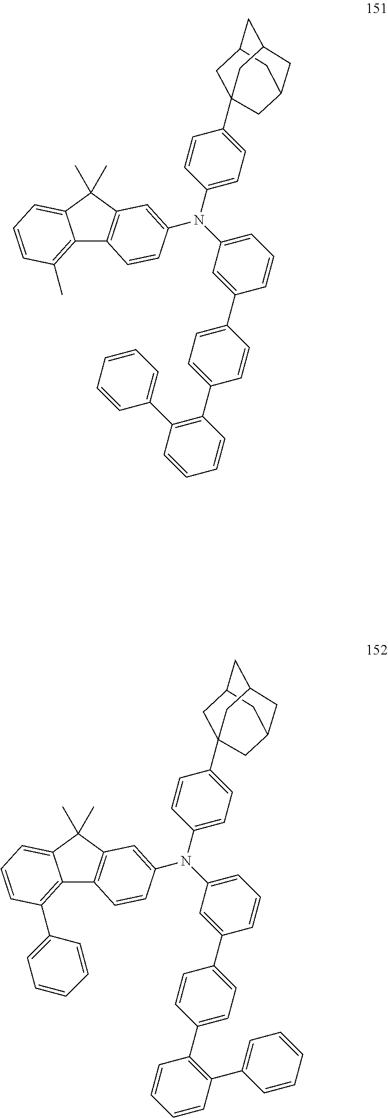 [SMILES: Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(C)C ; CC1(C)c2cc(N(c3ccc(C45CC6CC(CC(C6)C4)C5)cc3)c3cccc(-c4ccc(-c5ccccc5-c5ccccc5)cc4)c3)ccc2-c2c(-c3ccccc3)cccc21]

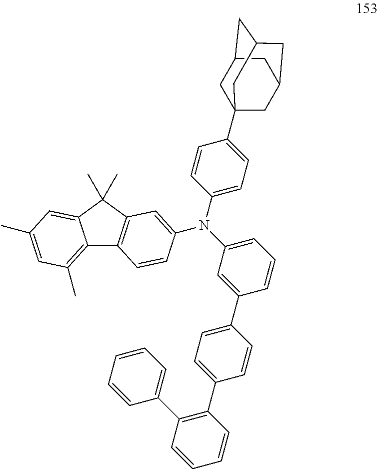 [SMILES: Cc1cc(C)c2c(c1)C(C)(C)c1cc(N(c3ccc(C45CC6CC(CC(C6)C4)C5)cc3)c3cccc(-c4ccc(-c5ccccc5-c5ccccc5)cc4)c3)ccc1-2]

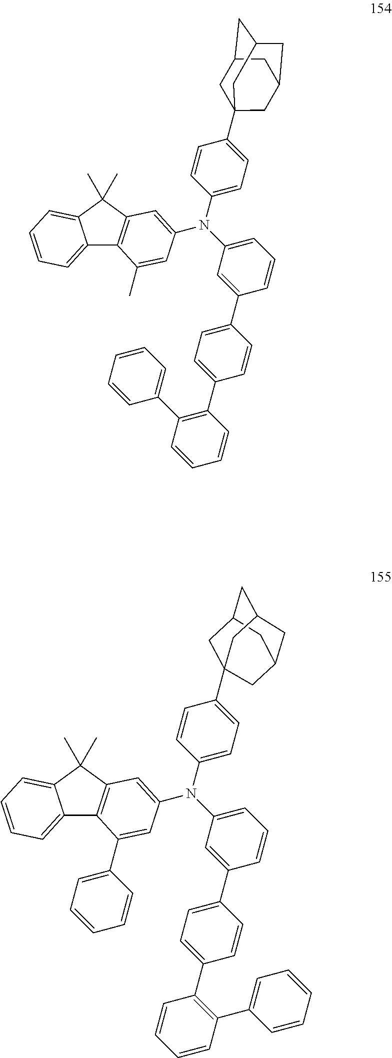 [SMILES: Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C45CC6CC(CC(C6)C4)C5)cc3)c3cccc(-c4ccc(-c5ccccc5-c5ccccc5)cc4)c3)cc21]

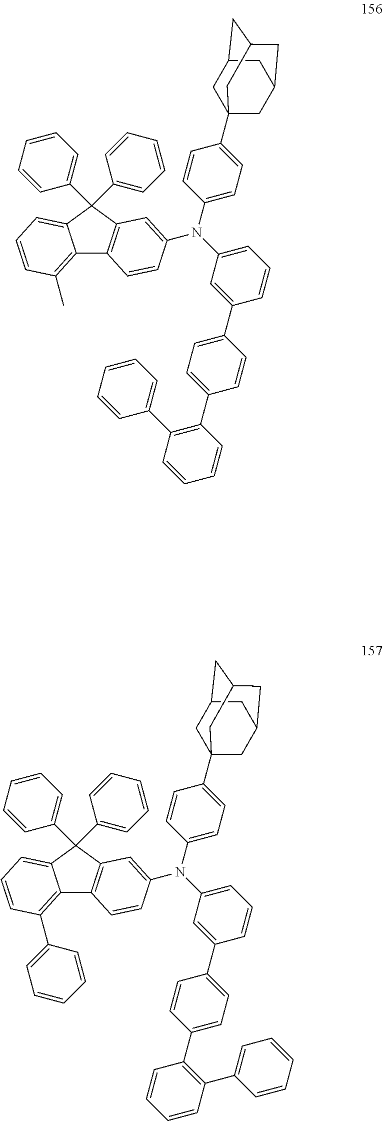 [SMILES: Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C56CC7CC(CC(C7)C5)C6)cc4)c4ccc5c(c4)C(c4ccccc4)(c4ccccc4)c4cccc(-c6ccccc6)c4-5)c3)cc2)cc1]

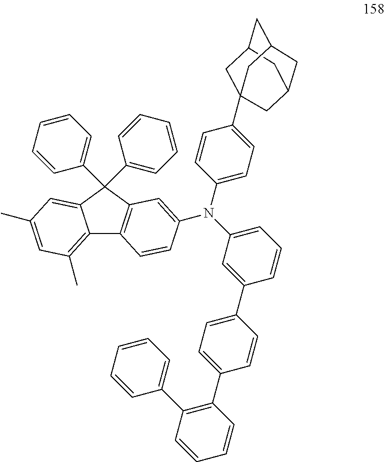 [SMILES: Cc1cc(C)c2c(c1)C(c1ccccc1)(c1ccccc1)c1cc(N(c3ccc(C45CC6CC(CC(C6)C4)C5)cc3)c3cccc(-c4ccc(-c5ccccc5-c5ccccc5)cc4)c3)ccc1-2]

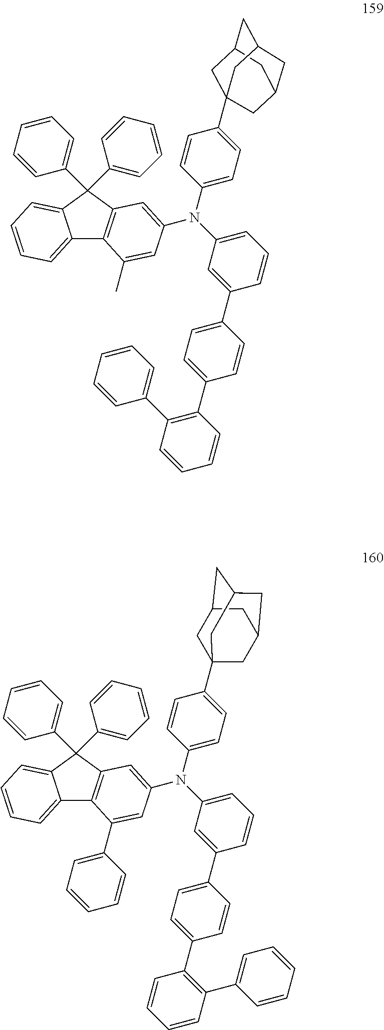 [SMILES: Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(c1ccccc1)c1ccccc1 ; c1ccc(-c2ccccc2-c2ccc(-c3cccc(N(c4ccc(C56CC7CC(CC(C7)C5)C6)cc4)c4cc(-c5ccccc5)c5c(c4)C(c4ccccc4)(c4ccccc4)c4ccccc4-5)c3)cc2)cc1]

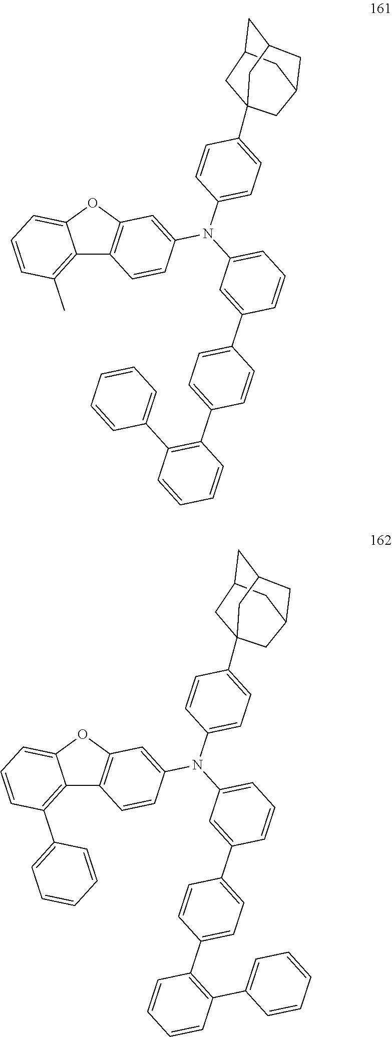 [SMILES: Cc1cccc2oc3cc(N(c4ccc(C56CC7CC(CC(C7)C5)C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C56CC7CC(CC(C7)C5)C6)cc4)c4ccc5c(c4)oc4cccc(-c6ccccc6)c45)c3)cc2)cc1]

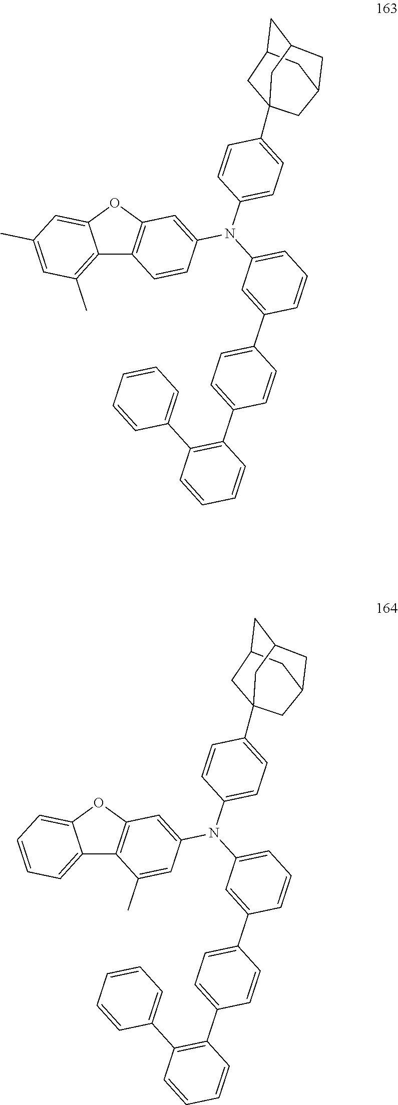 [SMILES: Cc1cc(C)c2c(c1)oc1cc(N(c3ccc(C45CC6CC(CC(C6)C4)C5)cc3)c3cccc(-c4ccc(-c5ccccc5-c5ccccc5)cc4)c3)ccc12 ; Cc1cc(N(c2ccc(C34CC5CC(CC(C5)C3)C4)cc2)c2cccc(-c3ccc(-c4ccccc4-c4ccccc4)cc3)c2)cc2oc3ccccc3c12]

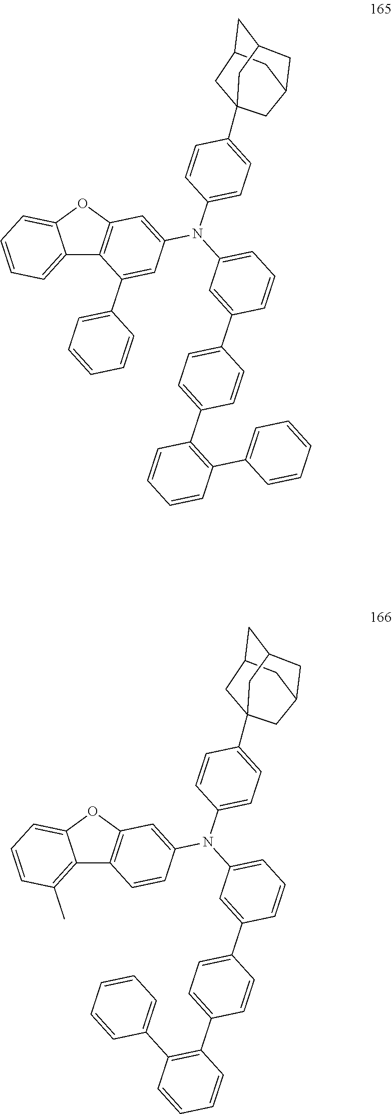 [SMILES: c1ccc(-c2ccccc2-c2ccc(-c3cccc(N(c4ccc(C56CC7CC(CC(C7)C5)C6)cc4)c4cc(-c5ccccc5)c5c(c4)oc4ccccc45)c3)cc2)cc1 ; Cc1cccc2oc3cc(N(c4ccc(C56CC7CC(CC(C7)C5)C6)cc4)c4cccc(-c5ccc(-c6ccccc6-c6ccccc6)cc5)c4)ccc3c12]

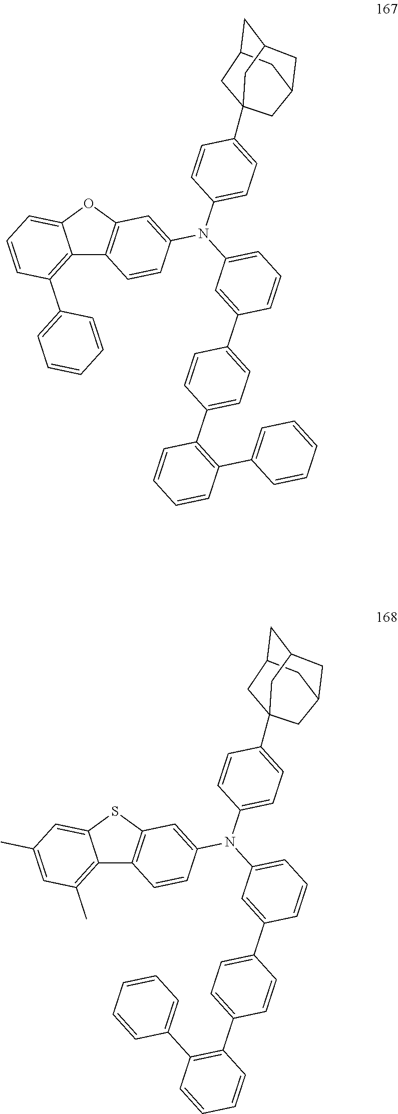 [SMILES: c1ccc(-c2ccccc2-c2ccc(-c3cccc(N(c4ccc(C56CC7CC(CC(C7)C5)C6)cc4)c4ccc5c(c4)oc4cccc(-c6ccccc6)c45)c3)cc2)cc1 ; Cc1cc(C)c2c(c1)sc1cc(N(c3ccc(C45CC6CC(CC(C6)C4)C5)cc3)c3cccc(-c4ccc(-c5ccccc5-c5ccccc5)cc4)c3)ccc12]

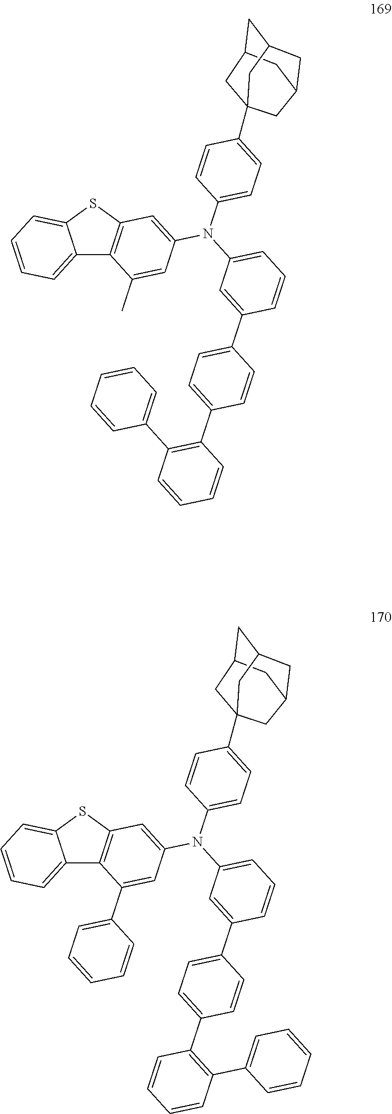 [SMILES: Cc1cc(N(c2ccc(C34CC5CC(CC(C5)C3)C4)cc2)c2cccc(-c3ccc(-c4ccccc4-c4ccccc4)cc3)c2)cc2sc3ccccc3c12 ; c1ccc(-c2ccccc2-c2ccc(-c3cccc(N(c4ccc(C56CC7CC(CC(C7)C5)C6)cc4)c4cc(-c5ccccc5)c5c(c4)sc4ccccc45)c3)cc2)cc1]

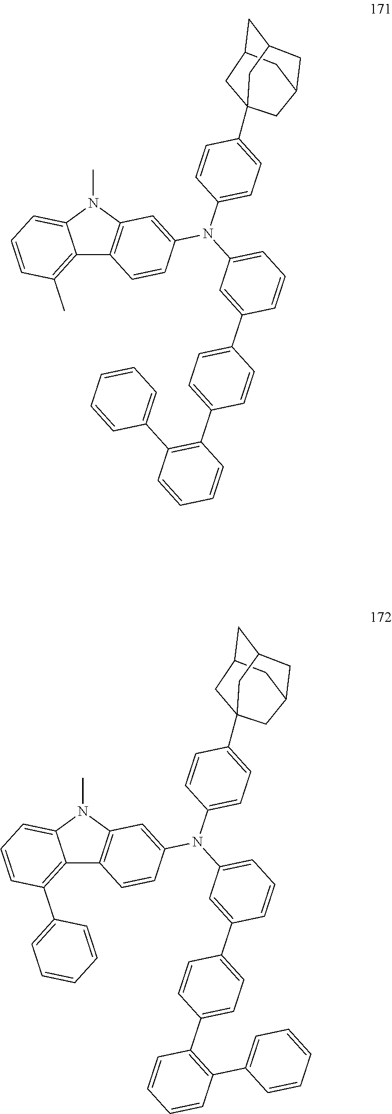 [SMILES: Cc1cccc2c1c1ccc(N(c3ccc(C45CC6CC(CC(C6)C4)C5)cc3)c3cccc(-c4ccc(-c5ccccc5-c5ccccc5)cc4)c3)cc1n2C ; Cn1c2cc(N(c3ccc(C45CC6CC(CC(C6)C4)C5)cc3)c3cccc(-c4ccc(-c5ccccc5-c5ccccc5)cc4)c3)ccc2c2c(-c3ccccc3)cccc21]

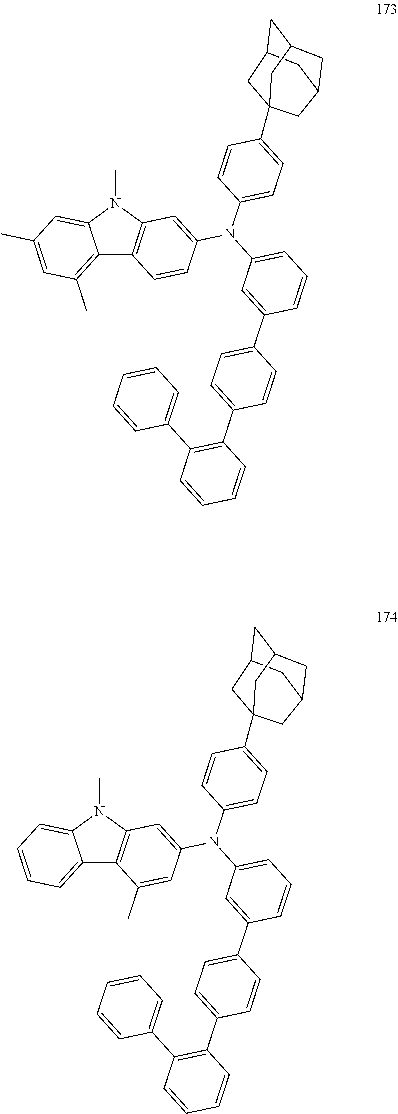 [SMILES: Cc1cc(C)c2c3ccc(N(c4ccc(C56CC7CC(CC(C7)C5)C6)cc4)c4cccc(-c5ccc(-c6ccccc6-c6ccccc6)cc5)c4)cc3n(C)c2c1 ; Cc1cc(N(c2ccc(C34CC5CC(CC(C5)C3)C4)cc2)c2cccc(-c3ccc(-c4ccccc4-c4ccccc4)cc3)c2)cc2c1c1ccccc1n2C]

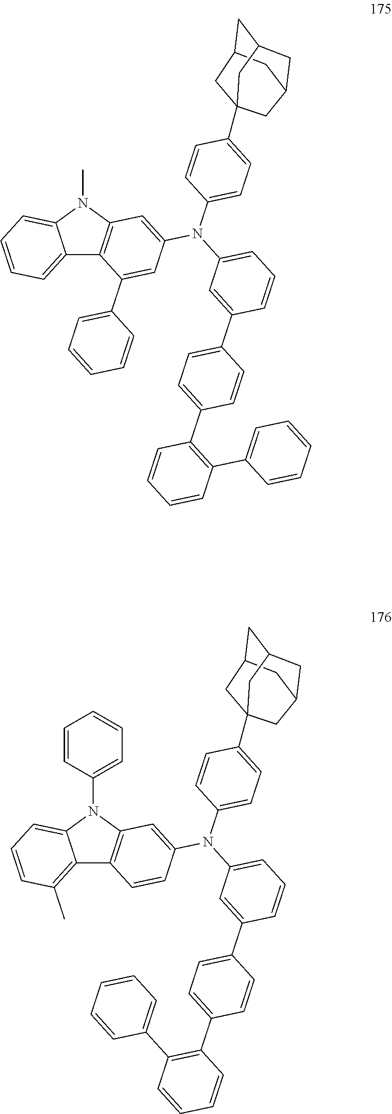 [SMILES: Cn1c2ccccc2c2c(-c3ccccc3)cc(N(c3ccc(C45CC6CC(CC(C6)C4)C5)cc3)c3cccc(-c4ccc(-c5ccccc5-c5ccccc5)cc4)c3)cc21 ; Cc1cccc2c1c1ccc(N(c3ccc(C45CC6CC(CC(C6)C4)C5)cc3)c3cccc(-c4ccc(-c5ccccc5-c5ccccc5)cc4)c3)cc1n2-c1ccccc1]

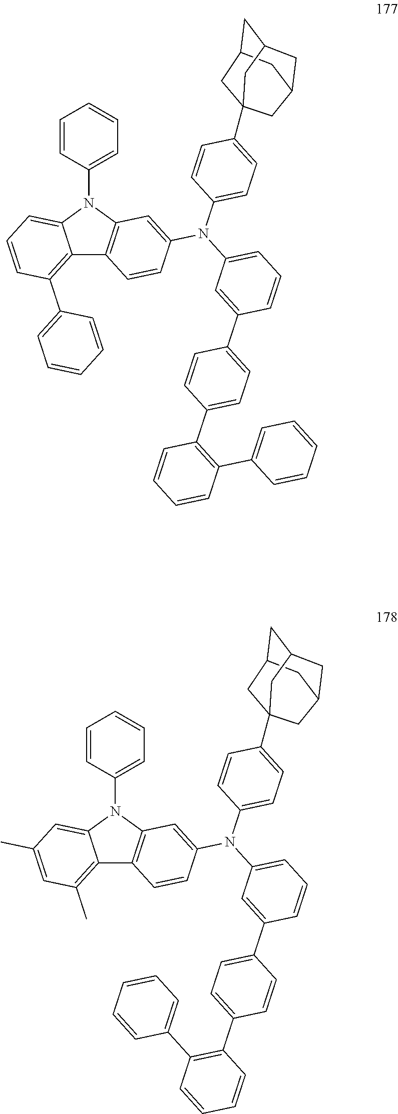 [SMILES: c1ccc(-c2ccccc2-c2ccc(-c3cccc(N(c4ccc(C56CC7CC(CC(C7)C5)C6)cc4)c4ccc5c6c(-c7ccccc7)cccc6n(-c6ccccc6)c5c4)c3)cc2)cc1 ; Cc1cc(C)c2c3ccc(N(c4ccc(C56CC7CC(CC(C7)C5)C6)cc4)c4cccc(-c5ccc(-c6ccccc6-c6ccccc6)cc5)c4)cc3n(-c3ccccc3)c2c1]

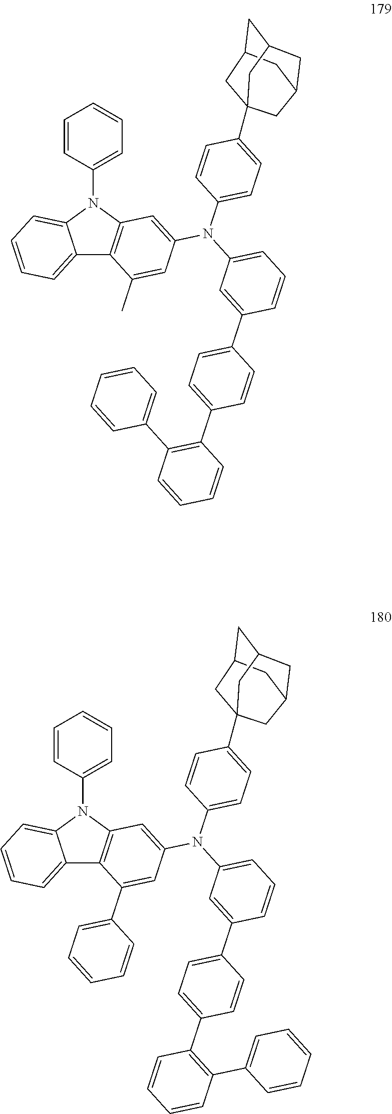 [SMILES: Cc1cc(N(c2ccc(C34CC5CC(CC(C5)C3)C4)cc2)c2cccc(-c3ccc(-c4ccccc4-c4ccccc4)cc3)c2)cc2c1c1ccccc1n2-c1ccccc1 ; c1ccc(-c2ccccc2-c2ccc(-c3cccc(N(c4ccc(C56CC7CC(CC(C7)C5)C6)cc4)c4cc(-c5ccccc5)c5c6ccccc6n(-c6ccccc6)c5c4)c3)cc2)cc1]

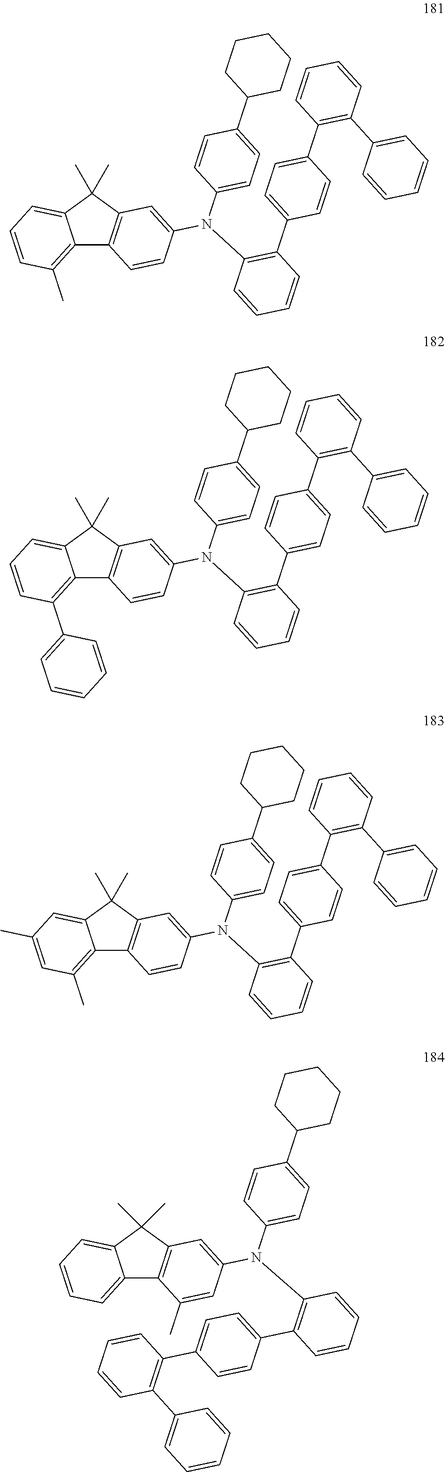 [SMILES: Cc1cccc2c1-c1ccc(N(c3ccc(C4CCCCC4)cc3)c3ccccc3-c3ccc(-c4ccccc4-c4ccccc4)cc3)cc1C2(C)C ; CC1(C)c2cc(N(c3ccc(C4CCCCC4)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CCCCC4)cc3)c3ccccc3-c3ccc(-c4ccccc4-c4ccccc4)cc3)ccc1-2 ; Cc1cc(N(c2ccc(C3CCCCC3)cc2)c2ccccc2-c2ccc(-c3ccccc3-c3ccccc3)cc2)cc2c1-c1ccccc1C2(C)C]

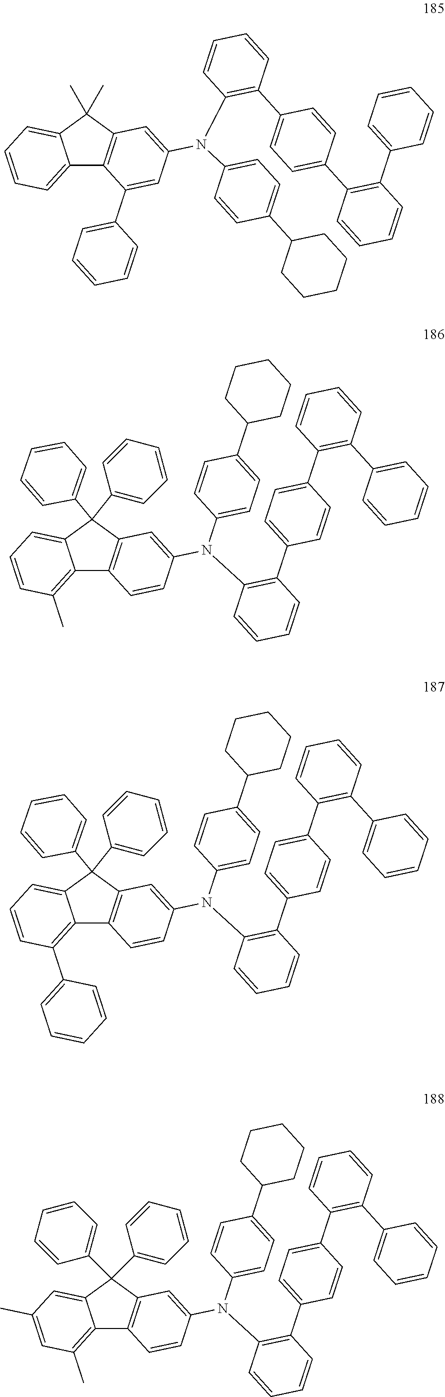 [SMILES: CC1(C)c2ccccc2-c2c(-c3ccccc3)cc(N(c3ccc(C4CCCCC4)cc3)c3ccccc3-c3ccc(-c4ccccc4-c4ccccc4)cc3)cc21 ; Cc1cccc2c1-c1ccc(N(c3ccc(C4CCCCC4)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C4CCCCC4)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C4CCCCC4)cc3)c3ccccc3-c3ccc(-c4ccccc4-c4ccccc4)cc3)ccc1-2]

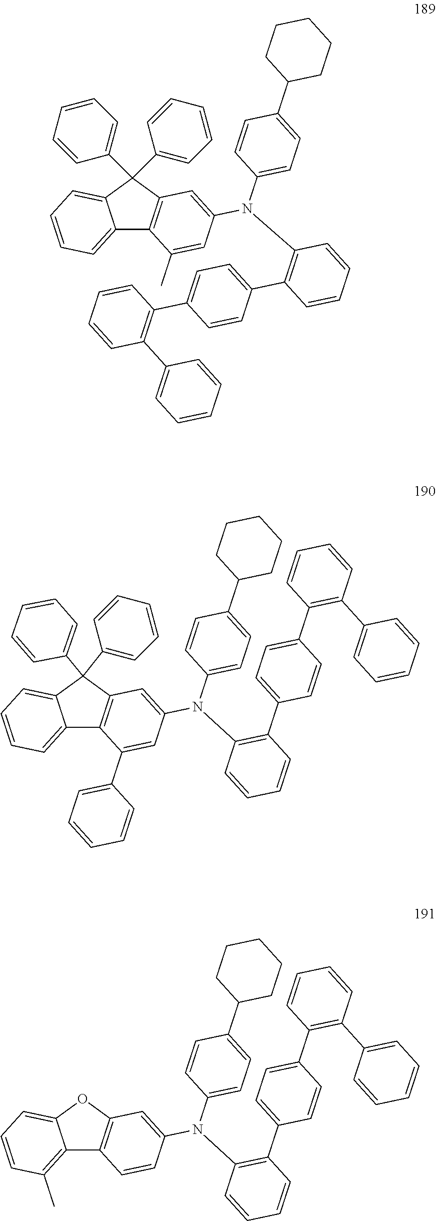 [SMILES: Cc1cc(N(c2ccc(C3CCCCC3)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C4CCCCC4)cc3)c3cc(-c4ccccc4)c4c(c3)C(c3ccccc3)(c3ccccc3)c3ccccc3-4)cc2)cc1 ; Cc1cccc2oc3cc(N(c4ccc(C5CCCCC5)cc4)c4ccccc4-c4ccc(-c5ccccc5-c5ccccc5)cc4)ccc3c12]

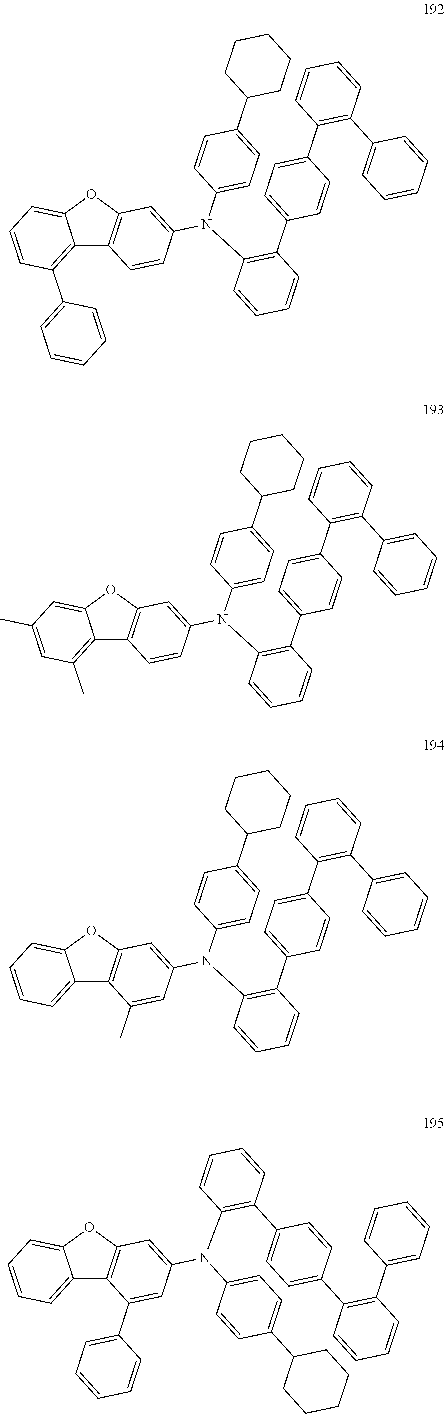 [SMILES: c1ccc(-c2ccccc2-c2ccc(-c3ccccc3N(c3ccc(C4CCCCC4)cc3)c3ccc4c(c3)oc3cccc(-c5ccccc5)c34)cc2)cc1 ; Cc1cc(C)c2c(c1)oc1cc(N(c3ccc(C4CCCCC4)cc3)c3ccccc3-c3ccc(-c4ccccc4-c4ccccc4)cc3)ccc12 ; Cc1cc(N(c2ccc(C3CCCCC3)cc2)c2ccccc2-c2ccc(-c3ccccc3-c3ccccc3)cc2)cc2oc3ccccc3c12 ; c1ccc(-c2ccccc2-c2ccc(-c3ccccc3N(c3ccc(C4CCCCC4)cc3)c3cc(-c4ccccc4)c4c(c3)oc3ccccc34)cc2)cc1]

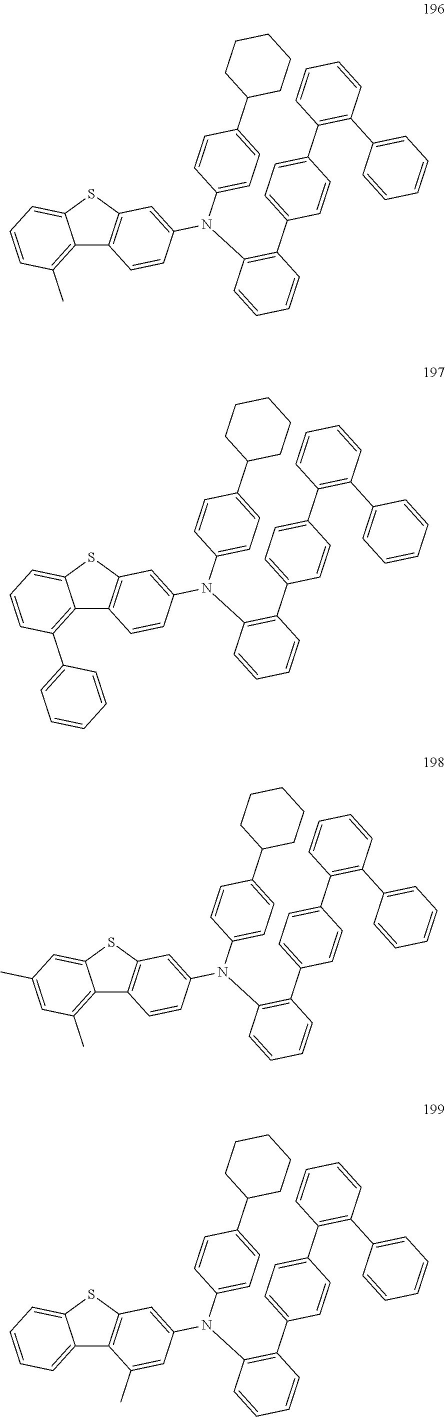 [SMILES: Cc1cccc2sc3cc(N(c4ccc(C5CCCCC5)cc4)c4ccccc4-c4ccc(-c5ccccc5-c5ccccc5)cc4)ccc3c12 ; c1ccc(-c2ccccc2-c2ccc(-c3ccccc3N(c3ccc(C4CCCCC4)cc3)c3ccc4c(c3)sc3cccc(-c5ccccc5)c34)cc2)cc1 ; Cc1cc(C)c2c(c1)sc1cc(N(c3ccc(C4CCCCC4)cc3)c3ccccc3-c3ccc(-c4ccccc4-c4ccccc4)cc3)ccc12 ; Cc1cc(N(c2ccc(C3CCCCC3)cc2)c2ccccc2-c2ccc(-c3ccccc3-c3ccccc3)cc2)cc2sc3ccccc3c12]

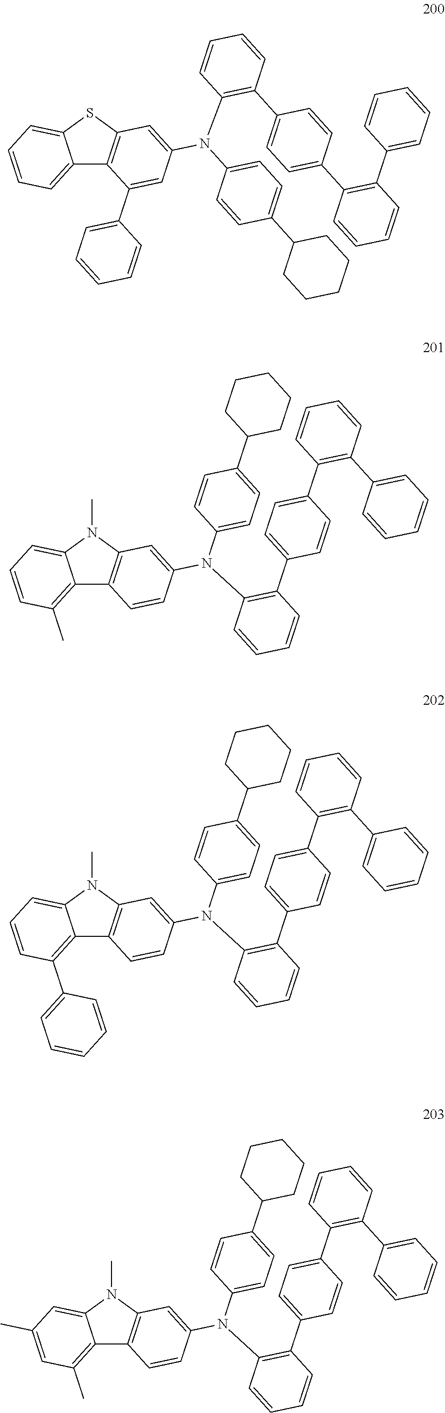 [SMILES: c1ccc(-c2ccccc2-c2ccc(-c3ccccc3N(c3ccc(C4CCCCC4)cc3)c3cc(-c4ccccc4)c4c(c3)sc3ccccc34)cc2)cc1 ; Cc1cccc2c1c1ccc(N(c3ccc(C4CCCCC4)cc3)c3ccccc3-c3ccc(-c4ccccc4-c4ccccc4)cc3)cc1n2C ; Cn1c2cc(N(c3ccc(C4CCCCC4)cc3)c3ccccc3-c3ccc(-c4ccccc4-c4ccccc4)cc3)ccc2c2c(-c3ccccc3)cccc21 ; Cc1cc(C)c2c3ccc(N(c4ccc(C5CCCCC5)cc4)c4ccccc4-c4ccc(-c5ccccc5-c5ccccc5)cc4)cc3n(C)c2c1]

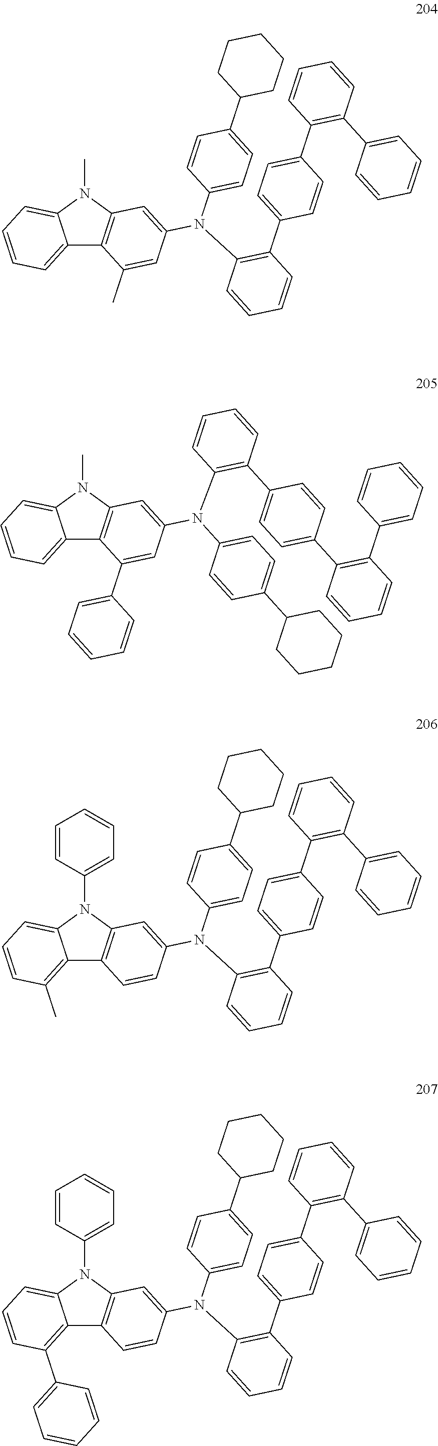 [SMILES: Cc1cc(N(c2ccc(C3CCCCC3)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C4CCCCC4)cc3)c3ccccc3-c3ccc(-c4ccccc4-c4ccccc4)cc3)cc21 ; Cc1cccc2c1c1ccc(N(c3ccc(C4CCCCC4)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C4CCCCC4)cc3)c3ccc4c5c(-c6ccccc6)cccc5n(-c5ccccc5)c4c3)cc2)cc1]

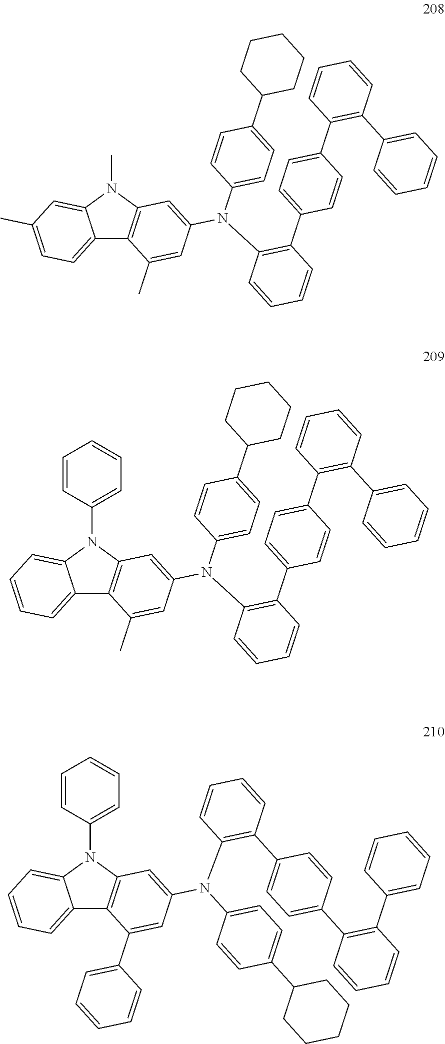 [SMILES: Cc1ccc2c3c(C)cc(N(c4ccc(C5CCCCC5)cc4)c4ccccc4-c4ccc(-c5ccccc5-c5ccccc5)cc4)cc3n(C)c2c1 ; Cc1cc(N(c2ccc(C3CCCCC3)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CCCCC4)cc3)c3cc(-c4ccccc4)c4c5ccccc5n(-c5ccccc5)c4c3)cc2)cc1]

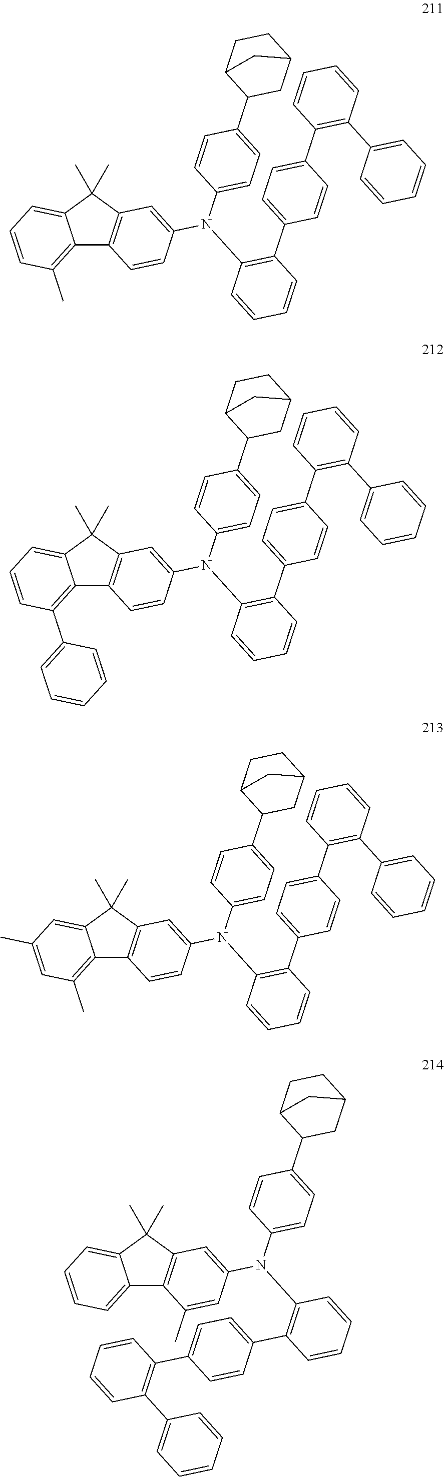 [SMILES: Cc1cccc2c1-c1ccc(N(c3ccc(C4CC5CCC4C5)cc3)c3ccccc3-c3ccc(-c4ccccc4-c4ccccc4)cc3)cc1C2(C)C ; CC1(C)c2cc(N(c3ccc(C4CC5CCC4C5)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CC5CCC4C5)cc3)c3ccccc3-c3ccc(-c4ccccc4-c4ccccc4)cc3)ccc1-2 ; Cc1cc(N(c2ccc(C3CC4CCC3C4)cc2)c2ccccc2-c2ccc(-c3ccccc3-c3ccccc3)cc2)cc2c1-c1ccccc1C2(C)C]

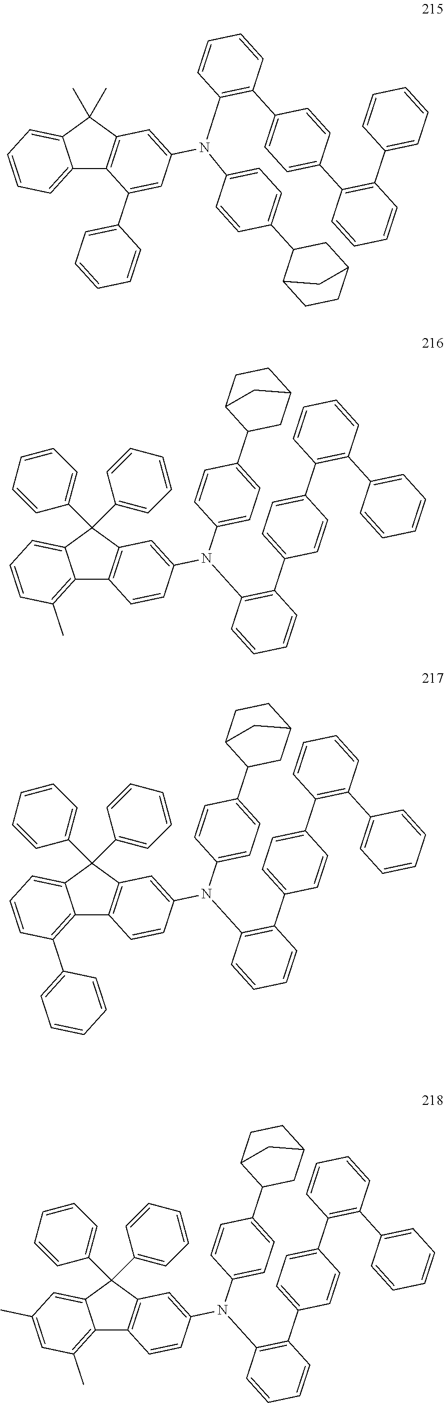 [SMILES: CC1(C)c2ccccc2-c2c(-c3ccccc3)cc(N(c3ccc(C4CC5CCC4C5)cc3)c3ccccc3-c3ccc(-c4ccccc4-c4ccccc4)cc3)cc21 ; Cc1cccc2c1-c1ccc(N(c3ccc(C4CC5CCC4C5)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C4CC5CCC4C5)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C4CC5CCC4C5)cc3)c3ccccc3-c3ccc(-c4ccccc4-c4ccccc4)cc3)ccc1-2]

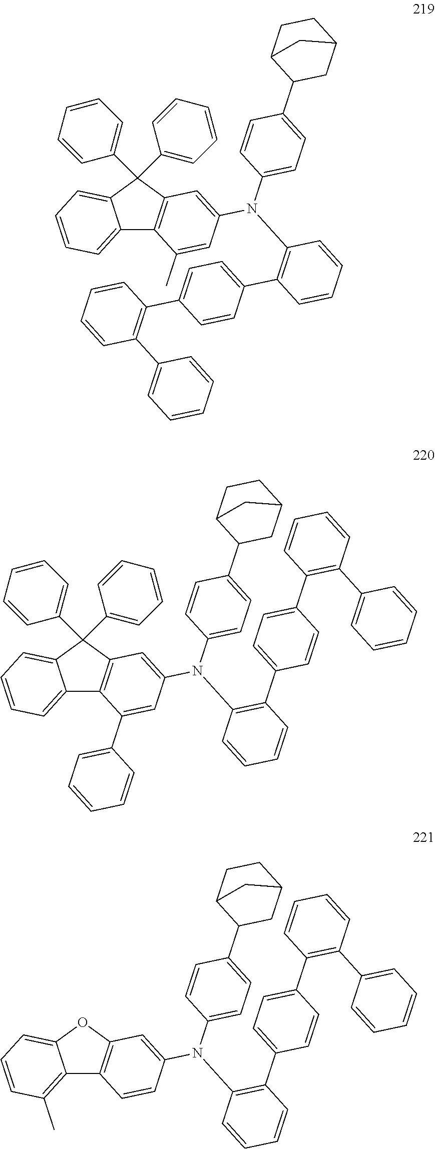 [SMILES: Cc1cc(N(c2ccc(C3CC4CCC3C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C4CC5CCC4C5)cc3)c3cc(-c4ccccc4)c4c(c3)C(c3ccccc3)(c3ccccc3)c3ccccc3-4)cc2)cc1 ; Cc1cccc2oc3cc(N(c4ccc(C5CC6CCC5C6)cc4)c4ccccc4-c4ccc(-c5ccccc5-c5ccccc5)cc4)ccc3c12]

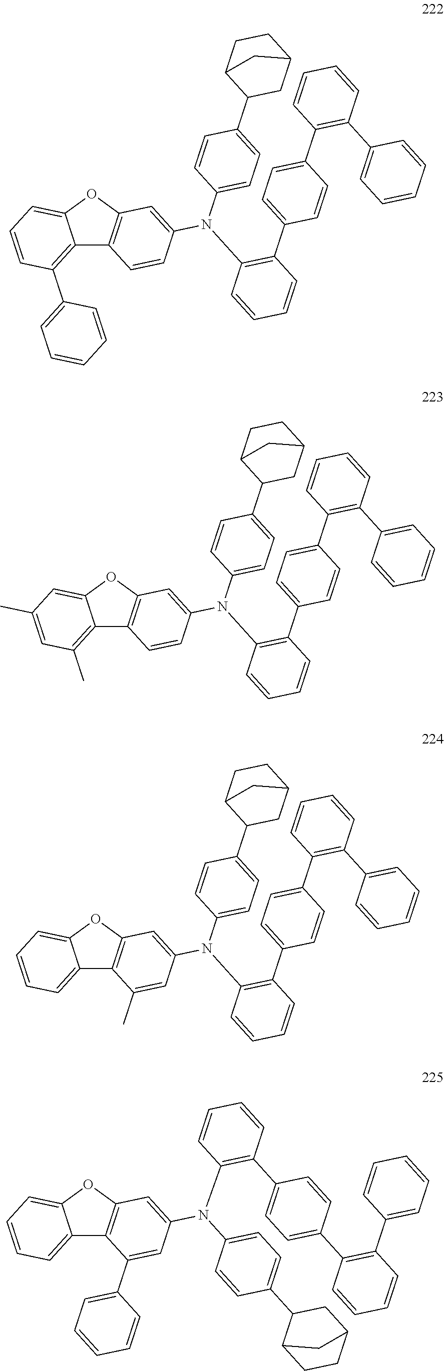 [SMILES: c1ccc(-c2ccccc2-c2ccc(-c3ccccc3N(c3ccc(C4CC5CCC4C5)cc3)c3ccc4c(c3)oc3cccc(-c5ccccc5)c34)cc2)cc1 ; Cc1cc(C)c2c(c1)oc1cc(N(c3ccc(C4CC5CCC4C5)cc3)c3ccccc3-c3ccc(-c4ccccc4-c4ccccc4)cc3)ccc12 ; Cc1cc(N(c2ccc(C3CC4CCC3C4)cc2)c2ccccc2-c2ccc(-c3ccccc3-c3ccccc3)cc2)cc2oc3ccccc3c12 ; c1ccc(-c2ccccc2-c2ccc(-c3ccccc3N(c3ccc(C4CC5CCC4C5)cc3)c3cc(-c4ccccc4)c4c(c3)oc3ccccc34)cc2)cc1]

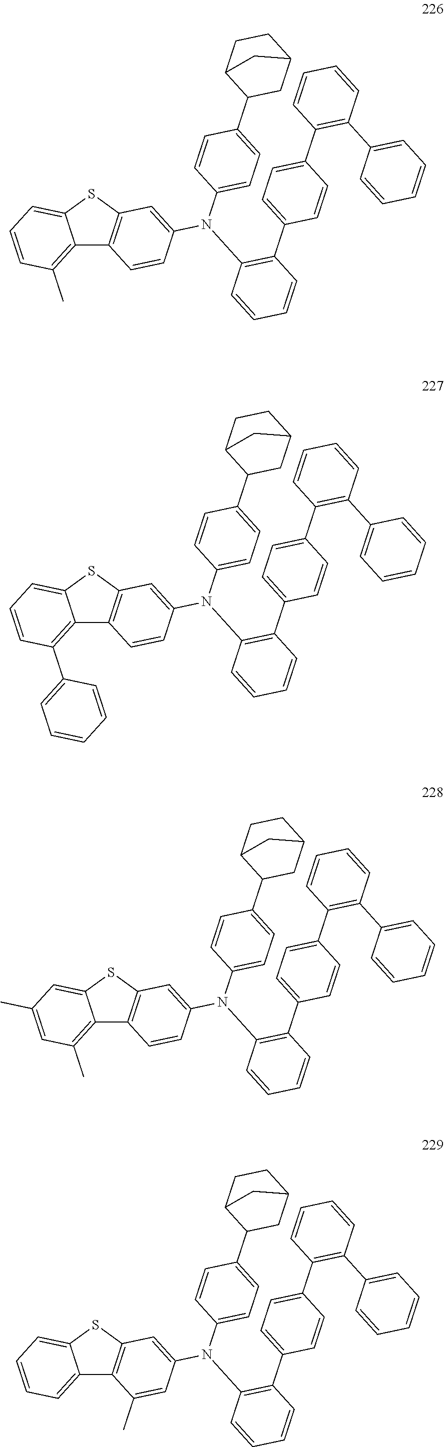 [SMILES: Cc1cccc2sc3cc(N(c4ccc(C5CC6CCC5C6)cc4)c4ccccc4-c4ccc(-c5ccccc5-c5ccccc5)cc4)ccc3c12 ; c1ccc(-c2ccccc2-c2ccc(-c3ccccc3N(c3ccc(C4CC5CCC4C5)cc3)c3ccc4c(c3)sc3cccc(-c5ccccc5)c34)cc2)cc1 ; Cc1cc(C)c2c(c1)sc1cc(N(c3ccc(C4CC5CCC4C5)cc3)c3ccccc3-c3ccc(-c4ccccc4-c4ccccc4)cc3)ccc12 ; Cc1cc(N(c2ccc(C3CC4CCC3C4)cc2)c2ccccc2-c2ccc(-c3ccccc3-c3ccccc3)cc2)cc2sc3ccccc3c12]

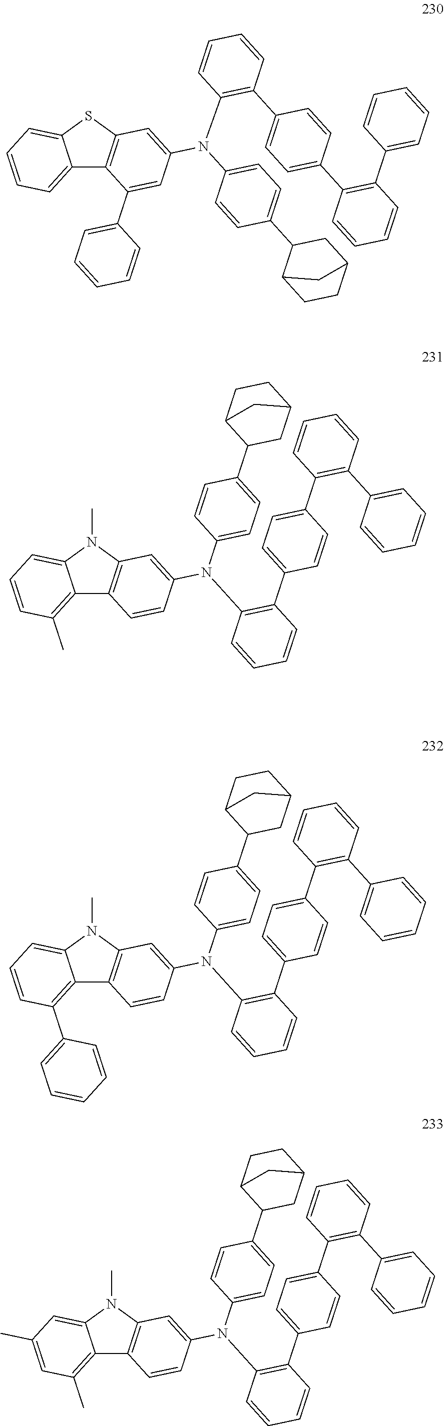 [SMILES: c1ccc(-c2ccccc2-c2ccc(-c3ccccc3N(c3ccc(C4CC5CCC4C5)cc3)c3cc(-c4ccccc4)c4c(c3)sc3ccccc34)cc2)cc1 ; Cc1cccc2c1c1ccc(N(c3ccc(C4CC5CCC4C5)cc3)c3ccccc3-c3ccc(-c4ccccc4-c4ccccc4)cc3)cc1n2C ; Cn1c2cc(N(c3ccc(C4CC5CCC4C5)cc3)c3ccccc3-c3ccc(-c4ccccc4-c4ccccc4)cc3)ccc2c2c(-c3ccccc3)cccc21 ; Cc1cc(C)c2c3ccc(N(c4ccc(C5CC6CCC5C6)cc4)c4ccccc4-c4ccc(-c5ccccc5-c5ccccc5)cc4)cc3n(C)c2c1]

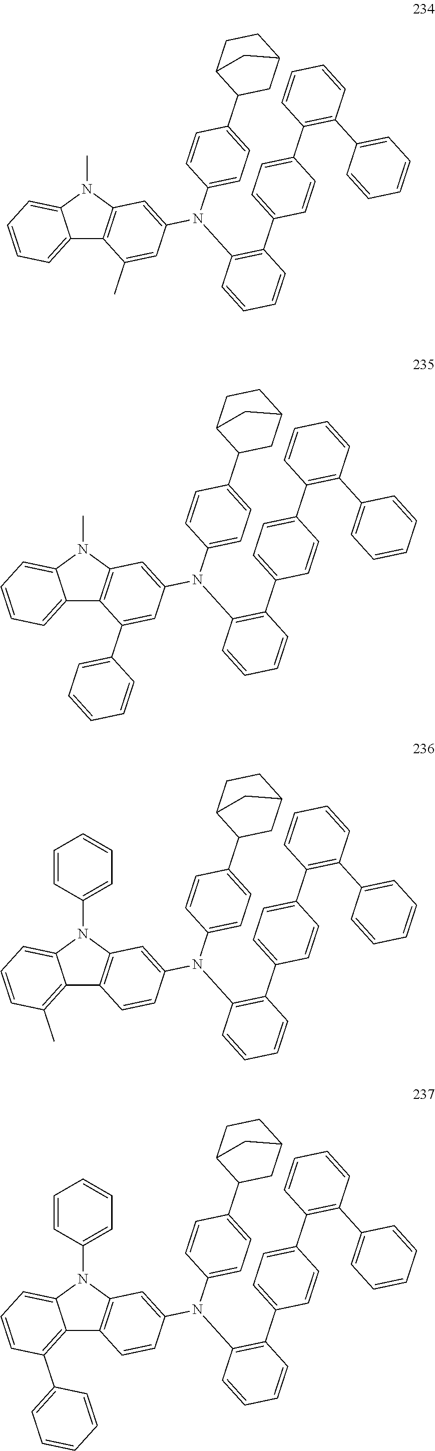 [SMILES: Cc1cc(N(c2ccc(C3CC4CCC3C4)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C4CC5CCC4C5)cc3)c3ccccc3-c3ccc(-c4ccccc4-c4ccccc4)cc3)cc21 ; Cc1cccc2c1c1ccc(N(c3ccc(C4CC5CCC4C5)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C4CC5CCC4C5)cc3)c3ccc4c5c(-c6ccccc6)cccc5n(-c5ccccc5)c4c3)cc2)cc1]

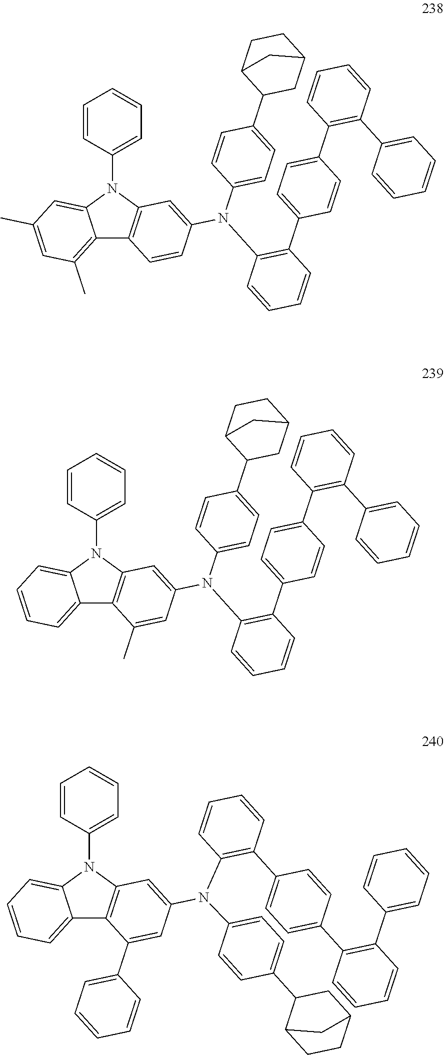 [SMILES: Cc1cc(C)c2c3ccc(N(c4ccc(C5CC6CCC5C6)cc4)c4ccccc4-c4ccc(-c5ccccc5-c5ccccc5)cc4)cc3n(-c3ccccc3)c2c1 ; Cc1cc(N(c2ccc(C3CC4CCC3C4)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CC5CCC4C5)cc3)c3cc(-c4ccccc4)c4c5ccccc5n(-c5ccccc5)c4c3)cc2)cc1]

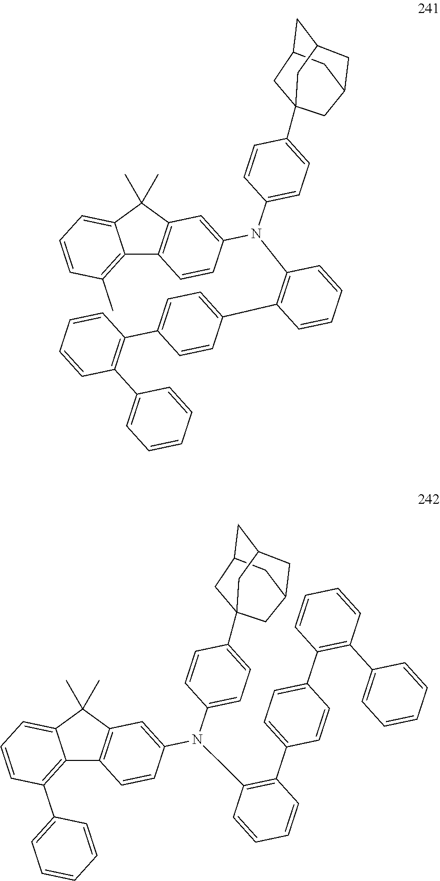 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(C)C ; CC1(C)c2cc(N(c3ccc(C45CC6CC(CC(C6)C4)C5)cc3)c3ccccc3-c3ccc(-c4ccccc4-c4ccccc4)cc3)ccc2-c2c(-c3ccccc3)cccc21]

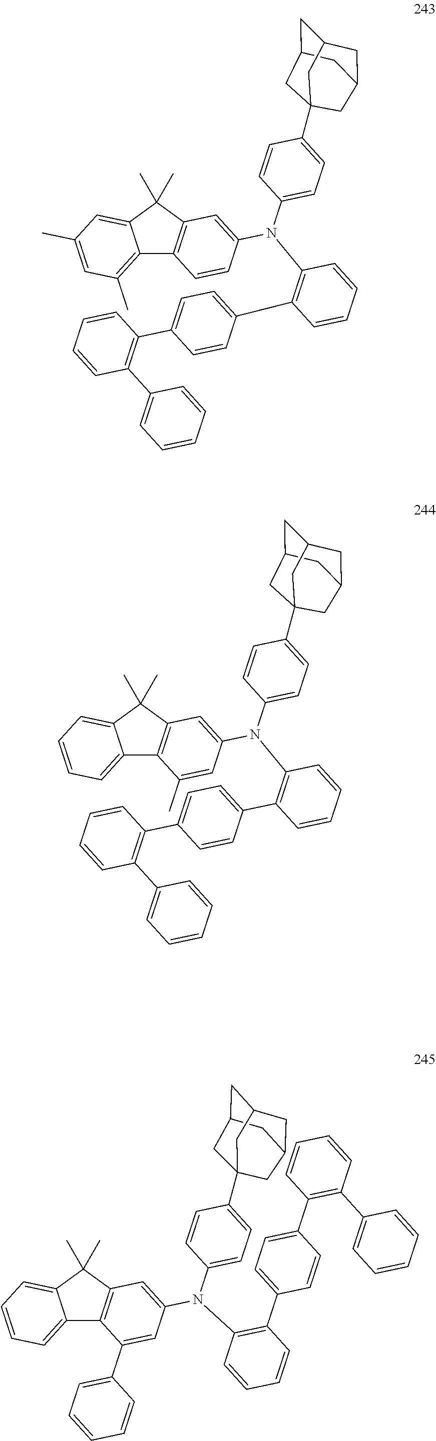 [SMILES: Cc1cc(C)c2c(c1)C(C)(C)c1cc(N(c3ccc(C45CC6CC(CC(C6)C4)C5)cc3)c3ccccc3-c3ccc(-c4ccccc4-c4ccccc4)cc3)ccc1-2 ; Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(C)C ; CC1(C)c2ccccc2-c2c(-c3ccccc3)cc(N(c3ccc(C45CC6CC(CC(C6)C4)C5)cc3)c3ccccc3-c3ccc(-c4ccccc4-c4ccccc4)cc3)cc21]

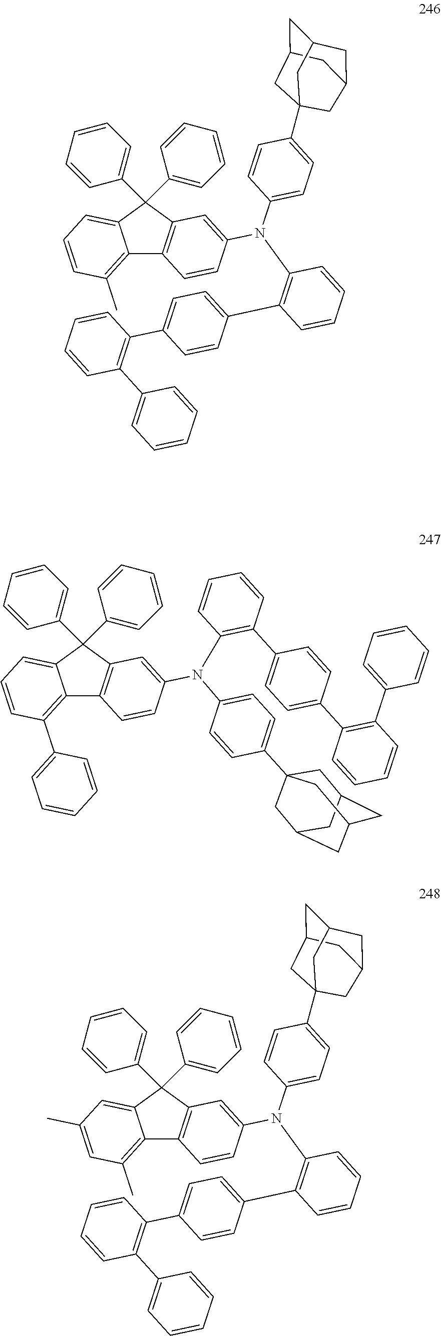 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C45CC6CC(CC(C6)C4)C5)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C45CC6CC(CC(C6)C4)C5)cc3)c3ccccc3-c3ccc(-c4ccccc4-c4ccccc4)cc3)ccc1-2]

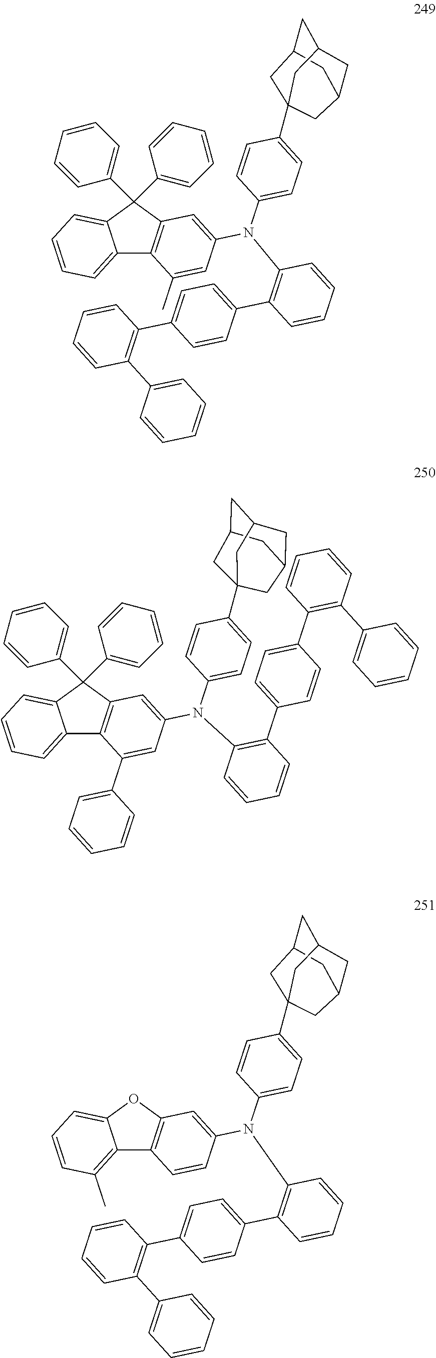 [SMILES: Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C45CC6CC(CC(C6)C4)C5)cc3)c3cc(-c4ccccc4)c4c(c3)C(c3ccccc3)(c3ccccc3)c3ccccc3-4)cc2)cc1 ; Cc1cccc2oc3cc(N(c4ccc(C56CC7CC(CC(C7)C5)C6)cc4)c4ccccc4-c4ccc(-c5ccccc5-c5ccccc5)cc4)ccc3c12]

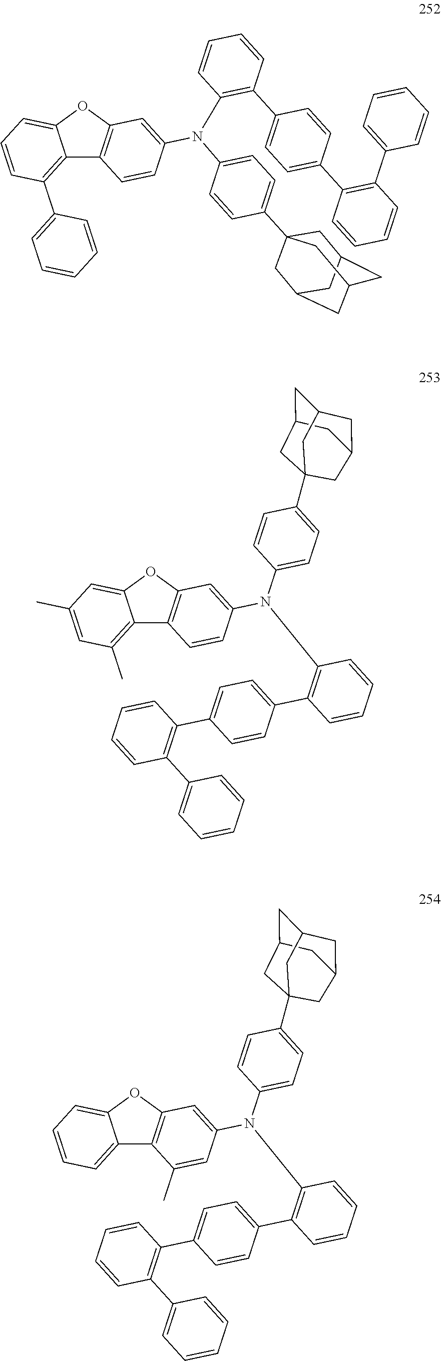 [SMILES: c1ccc(-c2ccccc2-c2ccc(-c3ccccc3N(c3ccc(C45CC6CC(CC(C6)C4)C5)cc3)c3ccc4c(c3)oc3cccc(-c5ccccc5)c34)cc2)cc1 ; Cc1cc(C)c2c(c1)oc1cc(N(c3ccc(C45CC6CC(CC(C6)C4)C5)cc3)c3ccccc3-c3ccc(-c4ccccc4-c4ccccc4)cc3)ccc12 ; Cc1cc(N(c2ccc(C34CC5CC(CC(C5)C3)C4)cc2)c2ccccc2-c2ccc(-c3ccccc3-c3ccccc3)cc2)cc2oc3ccccc3c12]

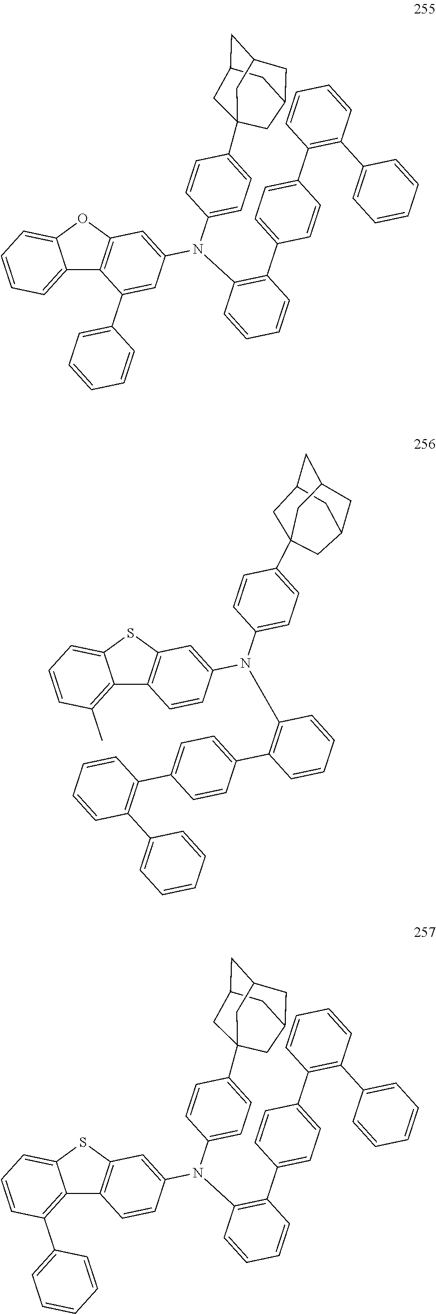 [SMILES: c1ccc(-c2ccccc2-c2ccc(-c3ccccc3N(c3ccc(C45CC6CC(CC(C6)C4)C5)cc3)c3cc(-c4ccccc4)c4c(c3)oc3ccccc34)cc2)cc1 ; Cc1cccc2sc3cc(N(c4ccc(C56CC7CC(CC(C7)C5)C6)cc4)c4ccccc4-c4ccc(-c5ccccc5-c5ccccc5)cc4)ccc3c12 ; c1ccc(-c2ccccc2-c2ccc(-c3ccccc3N(c3ccc(C45CC6CC(CC(C6)C4)C5)cc3)c3ccc4c(c3)sc3cccc(-c5ccccc5)c34)cc2)cc1]

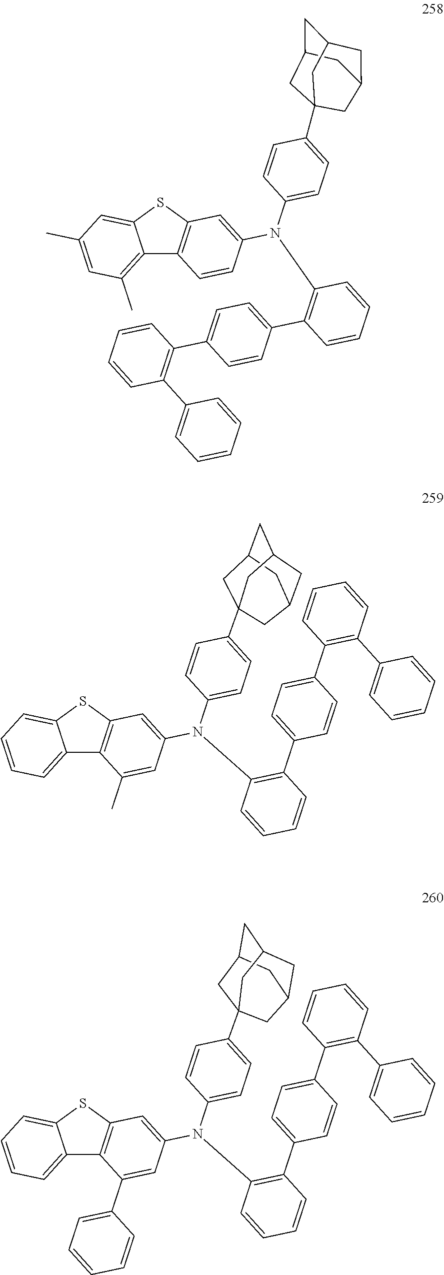 [SMILES: Cc1cc(C)c2c(c1)sc1cc(N(c3ccc(C45CC6CC(CC(C6)C4)C5)cc3)c3ccccc3-c3ccc(-c4ccccc4-c4ccccc4)cc3)ccc12 ; Cc1cc(N(c2ccc(C34CC5CC(CC(C5)C3)C4)cc2)c2ccccc2-c2ccc(-c3ccccc3-c3ccccc3)cc2)cc2sc3ccccc3c12 ; c1ccc(-c2ccccc2-c2ccc(-c3ccccc3N(c3ccc(C45CC6CC(CC(C6)C4)C5)cc3)c3cc(-c4ccccc4)c4c(c3)sc3ccccc34)cc2)cc1]

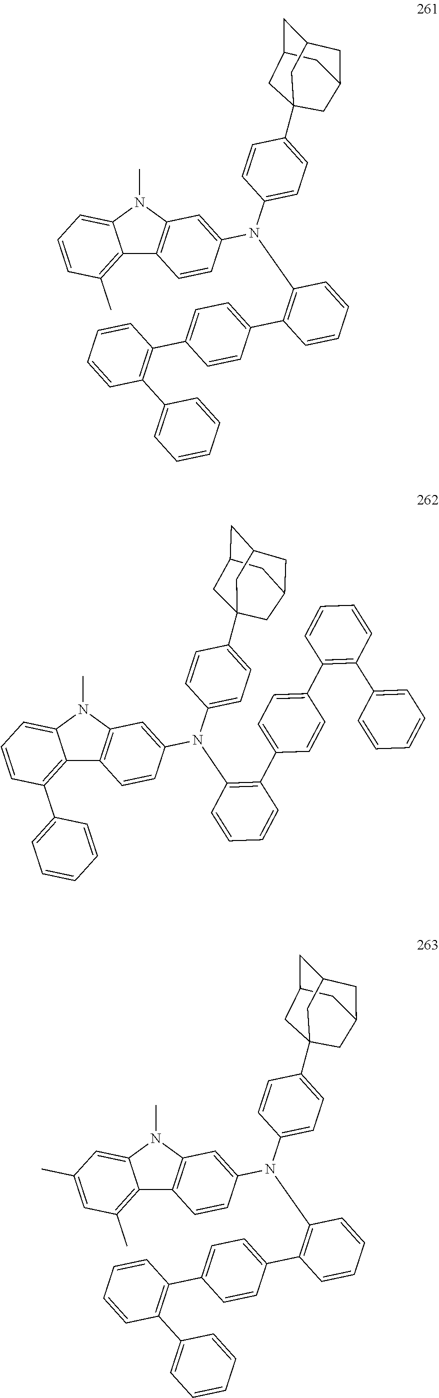 [SMILES: Cc1cccc2c1c1ccc(N(c3ccc(C45CC6CC(CC(C6)C4)C5)cc3)c3ccccc3-c3ccc(-c4ccccc4-c4ccccc4)cc3)cc1n2C ; Cn1c2cc(N(c3ccc(C45CC6CC(CC(C6)C4)C5)cc3)c3ccccc3-c3ccc(-c4ccccc4-c4ccccc4)cc3)ccc2c2c(-c3ccccc3)cccc21 ; Cc1cc(C)c2c3ccc(N(c4ccc(C56CC7CC(CC(C7)C5)C6)cc4)c4ccccc4-c4ccc(-c5ccccc5-c5ccccc5)cc4)cc3n(C)c2c1]

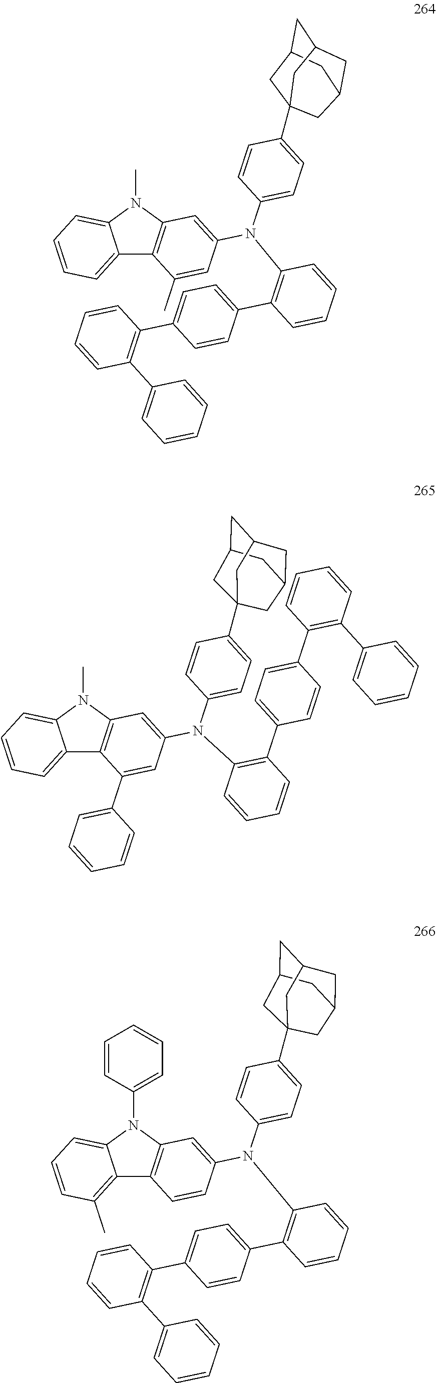 [SMILES: Cc1cc(N(c2ccc(C34CC5CC(CC(C5)C3)C4)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C45CC6CC(CC(C6)C4)C5)cc3)c3ccccc3-c3ccc(-c4ccccc4-c4ccccc4)cc3)cc21 ; Cc1cccc2c1c1ccc(N(c3ccc(C45CC6CC(CC(C6)C4)C5)cc3)c3ccccc3-c3ccc(-c4ccccc4-c4ccccc4)cc3)cc1n2-c1ccccc1]

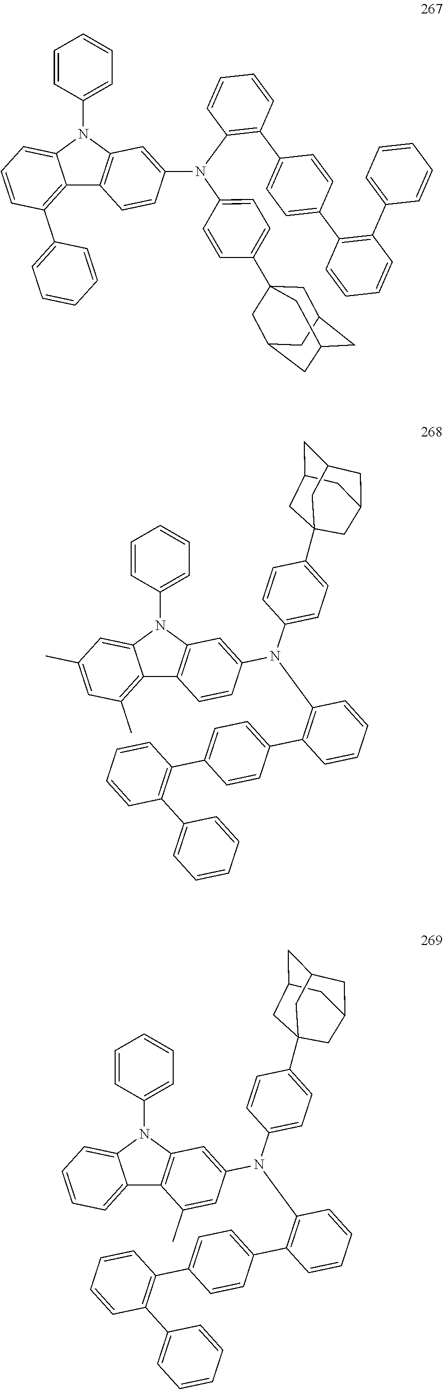 [SMILES: c1ccc(-c2ccccc2-c2ccc(-c3ccccc3N(c3ccc(C45CC6CC(CC(C6)C4)C5)cc3)c3ccc4c5c(-c6ccccc6)cccc5n(-c5ccccc5)c4c3)cc2)cc1 ; Cc1cc(C)c2c3ccc(N(c4ccc(C56CC7CC(CC(C7)C5)C6)cc4)c4ccccc4-c4ccc(-c5ccccc5-c5ccccc5)cc4)cc3n(-c3ccccc3)c2c1 ; Cc1cc(N(c2ccc(C34CC5CC(CC(C5)C3)C4)cc2)c2ccccc2-c2ccc(-c3ccccc3-c3ccccc3)cc2)cc2c1c1ccccc1n2-c1ccccc1]

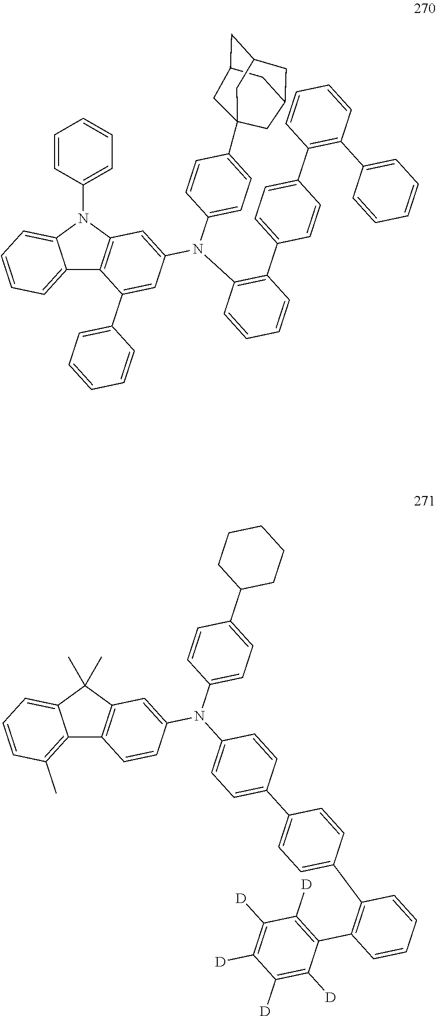 [SMILES: c1ccc(-c2ccccc2-c2ccc(-c3ccccc3N(c3ccc(C45CC6CC(CC(C6)C4)C5)cc3)c3cc(-c4ccccc4)c4c5ccccc5n(-c5ccccc5)c4c3)cc2)cc1 ; [2H]c1c([2H])c([2H])c(-c2ccccc2-c2ccc(-c3ccc(N(c4ccc(C5CCCCC5)cc4)c4ccc5c(c4)C(C)(C)c4cccc(C)c4-5)cc3)cc2)c([2H])c1[2H]]

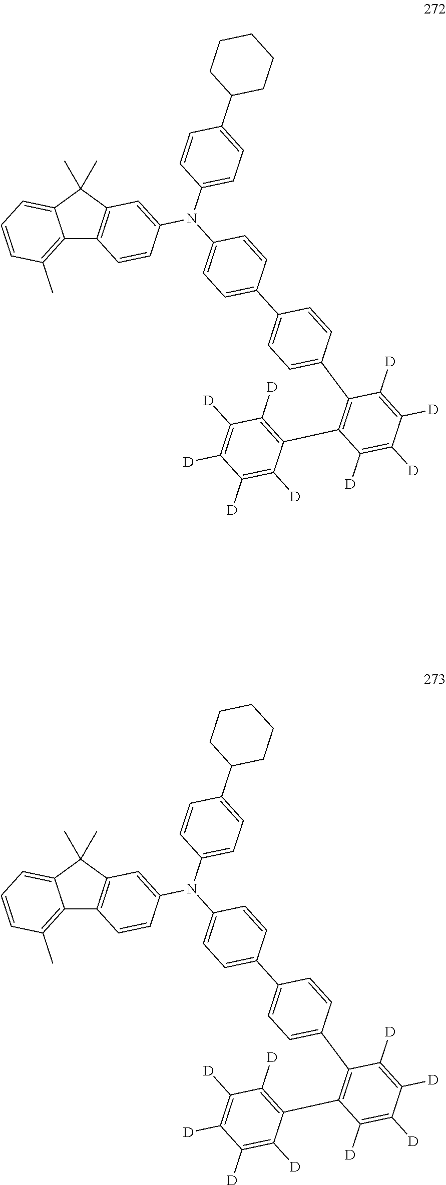 [SMILES: [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H] ; [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H]]

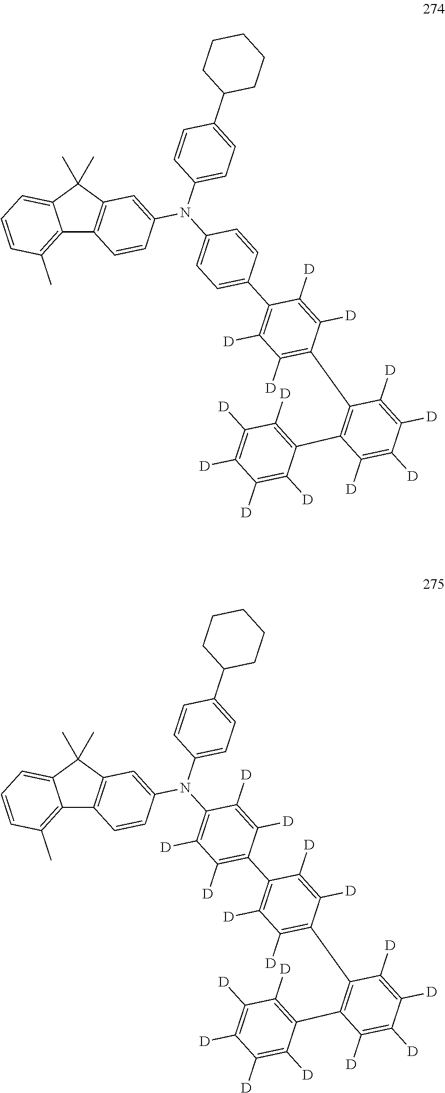 [SMILES: [2H]c1c([2H])c([2H])c(-c2c([2H])c([2H])c([2H])c([2H])c2-c2c([2H])c([2H])c(-c3ccc(N(c4ccc(C5CCCCC5)cc4)c4ccc5c(c4)C(C)(C)c4cccc(C)c4-5)cc3)c([2H])c2[2H])c([2H])c1[2H] ; [2H]c1c([2H])c([2H])c(-c2c([2H])c([2H])c([2H])c([2H])c2-c2c([2H])c([2H])c(-c3c([2H])c([2H])c(N(c4ccc(C5CCCCC5)cc4)c4ccc5c(c4)C(C)(C)c4cccc(C)c4-5)c([2H])c3[2H])c([2H])c2[2H])c([2H])c1[2H]]

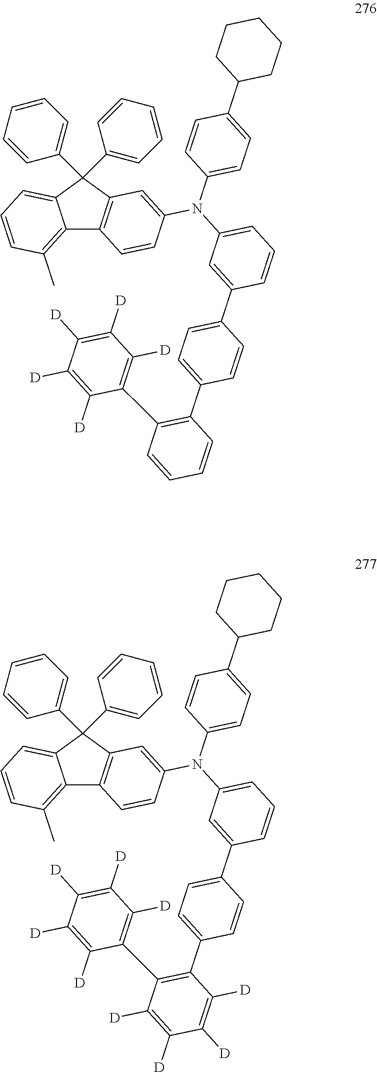 [SMILES: [2H]c1c([2H])c([2H])c(-c2ccccc2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H] ; [2H]c1c([2H])c([2H])c(-c2c([2H])c([2H])c([2H])c([2H])c2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H]]

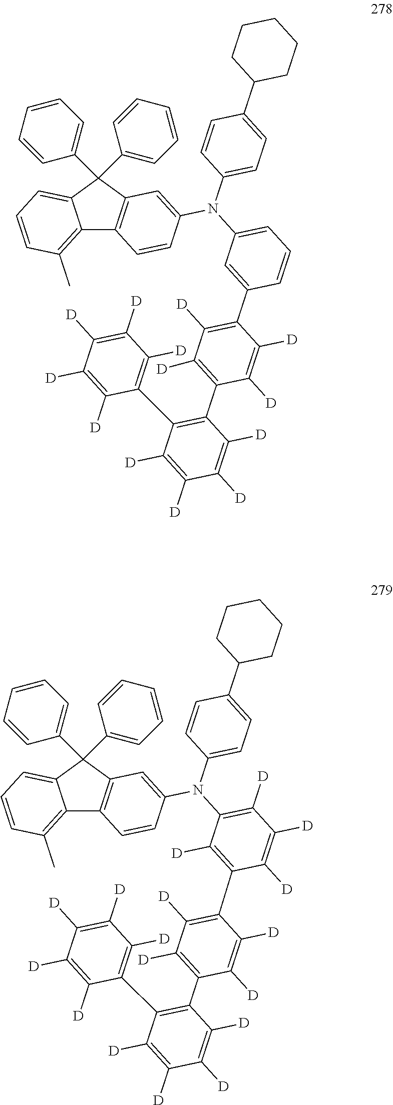 [SMILES: [2H]c1c([2H])c([2H])c(-c2c([2H])c([2H])c([2H])c([2H])c2-c2c([2H])c([2H])c(-c3cccc(N(c4ccc(C5CCCCC5)cc4)c4ccc5c(c4)C(c4ccccc4)(c4ccccc4)c4cccc(C)c4-5)c3)c([2H])c2[2H])c([2H])c1[2H] ; [2H]c1c([2H])c([2H])c(-c2c([2H])c([2H])c([2H])c([2H])c2-c2c([2H])c([2H])c(-c3c([2H])c([2H])c([2H])c(N(c4ccc(C5CCCCC5)cc4)c4ccc5c(c4)C(c4ccccc4)(c4ccccc4)c4cccc(C)c4-5)c3[2H])c([2H])c2[2H])c([2H])c1[2H]]

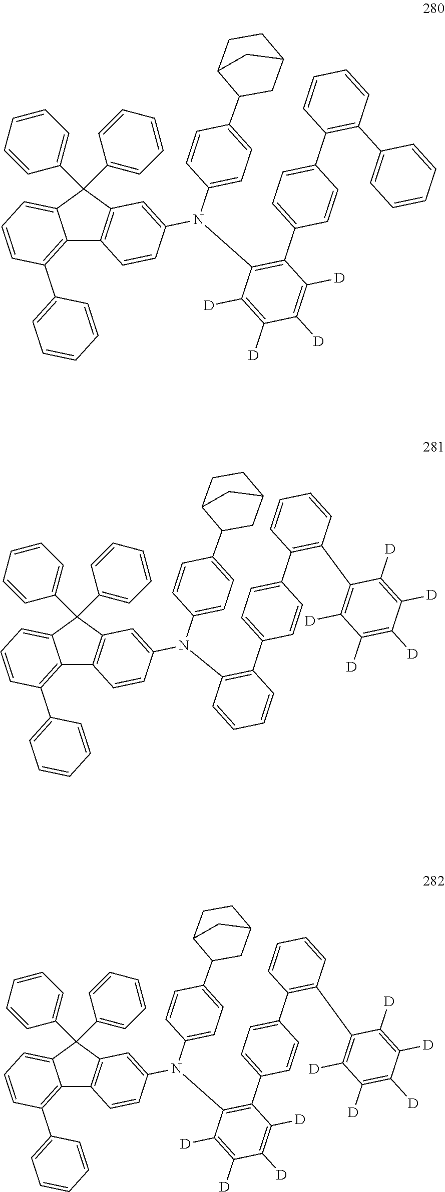 [SMILES: [2H]c1c([2H])c([2H])c(N(c2ccc(C3CC4CCC3C4)cc2)c2ccc3c(c2)C(c2ccccc2)(c2ccccc2)c2cccc(-c4ccccc4)c2-3)c(-c2ccc(-c3ccccc3-c3ccccc3)cc2)c1[2H] ; [2H]c1c([2H])c([2H])c(-c2ccccc2-c2ccc(-c3ccccc3N(c3ccc(C4CC5CCC4C5)cc3)c3ccc4c(c3)C(c3ccccc3)(c3ccccc3)c3cccc(-c5ccccc5)c3-4)cc2)c([2H])c1[2H] ; [2H]c1c([2H])c([2H])c(-c2ccccc2-c2ccc(-c3c([2H])c([2H])c([2H])c([2H])c3N(c3ccc(C4CC5CCC4C5)cc3)c3ccc4c(c3)C(c3ccccc3)(c3ccccc3)c3cccc(-c5ccccc5)c3-4)cc2)c([2H])c1[2H]]

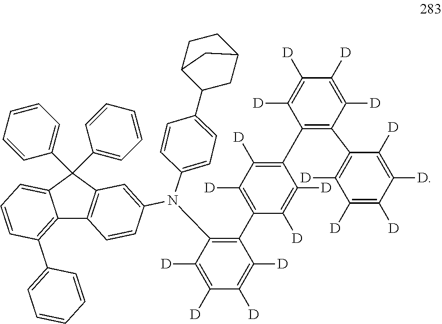

15. A display device comprising:
a base layer;
a circuit layer on the base layer; and
a display element layer on the circuit layer and comprising a light-emitting element,
wherein the light-emitting element comprises a first electrode, a second electrode on the first electrode, and a hole transport region between the first electrode and the second electrode and comprising an amine compound represented by Formula 1: 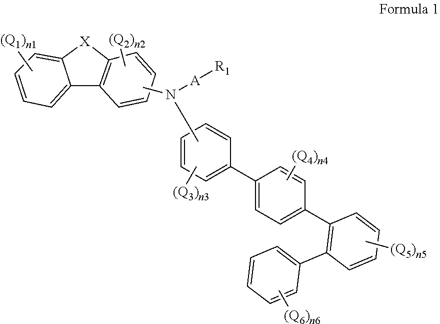
wherein, in Formula 1,
n1, n3, n4, and n5 are each independently an integer of 0 to 4,
n2 is an integer of 0 to 3,
n6 is an integer of 0 to 5,
n1 and n2 are not 0 at the same time,
X is O, S, NR₂, or CR₃R₄,
A is a substituted or unsubstituted arylene group having 6 to 30 ring-forming carbon atoms,
R₁ is a substituted or unsubstituted cycloalkyl group having 3 to 10 ring-forming carbon atoms,
R₂, R₃, and R₄ are each independently a hydrogen atom, a deuterium atom, a substituted or unsubstituted alkyl group having 1 to 30 carbon atoms, or a substituted or unsubstituted aryl group having 6 to 30 ring-forming carbon atoms,
Q₁ and Q₂ are each independently a substituted or unsubstituted alkyl group having 1 to 10 carbon atoms or a substituted or unsubstituted aryl group having 6 to 30 ring-forming carbon atoms, and
Q₃, Q₄, Q₅, and Q₆ are each independently a hydrogen atom, a deuterium atom,
a substituted or unsubstituted alkyl group having 1 to 10 carbon atoms, or a substituted or unsubstituted aryl group having 6 to 30 ring-forming carbon atoms.

16. An amine compound represented by Formula 1: 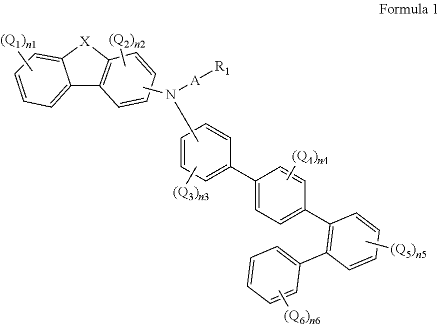
wherein, in Formula 1
n1, n3, n4, and n5 are each independently an integer of 0 to 4,
n2 is an integer of 0 to 3,
n6 is an integer of 0 to 5,
n1 and n2 are not 0 at the same time,
X is O, S, NR₂, or CR₃R₄,
A is a substituted or unsubstituted arylene group having 6 to 30 ring-forming carbon atoms,
R₁ is a substituted or unsubstituted cycloalkyl group having 3 to 10 ring-forming carbon atoms,
R₂, R₃, and R₄ are each independently a hydrogen atom, a deuterium atom, a substituted or unsubstituted alkyl group having 1 to 30 carbon atoms, or a substituted or unsubstituted aryl group having 6 to 30 ring-forming carbon atoms,
Q₁ and Q₂ are each independently a substituted or unsubstituted alkyl group having 1 to 10 carbon atoms or a substituted or unsubstituted aryl group having 6 to 30 ring-forming carbon atoms, and
Q₃, Q₄, Q₅, and Q₆ are each independently a hydrogen atom, a deuterium atom,
a substituted or unsubstituted alkyl group having 1 to 10 carbon atoms, or a substituted or unsubstituted aryl group having 6 to 30 ring-forming carbon atoms.

17. The amine compound of claim 16,
wherein the amine compound represented by Formula 1 is represented by Formula 1-1: 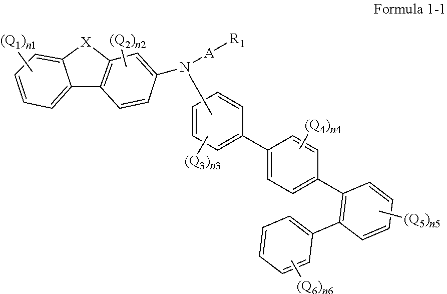
wherein, in Formula 1-1,
n1 to n6, X, A, R₁ to R₄, and Q₁ to Q₆ are the same as defined in the descriptions of Formula 1.

18. The amine compound of claim 1,
wherein the amine compound represented by Formula 1 is represented by any one selected from among Formulas 2-1 to 2-3: 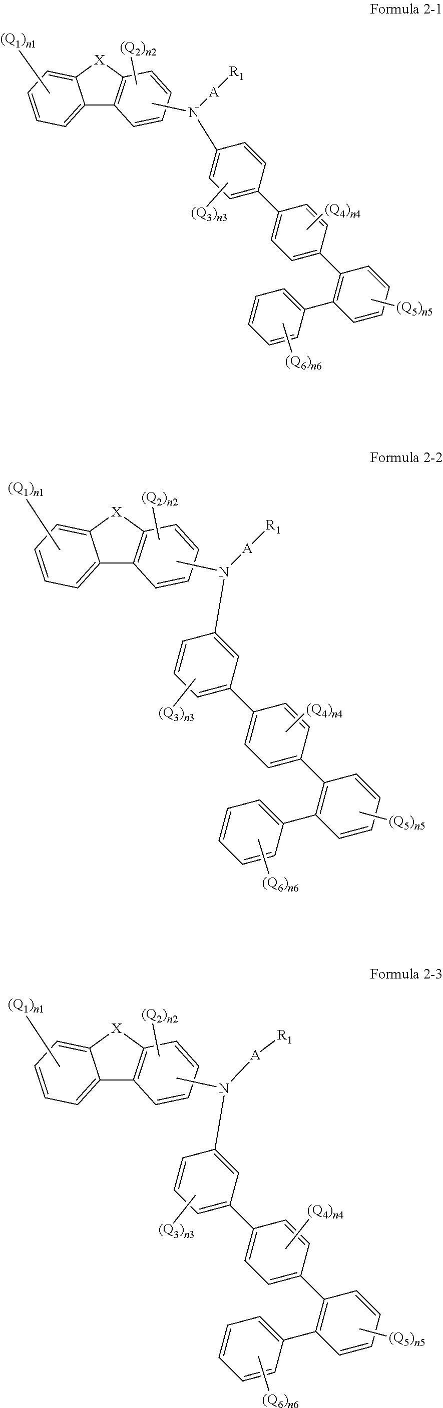
wherein, in Formulas 2-1 to 2-3,
n1 to n6, X, A, R₁ to R₄, and Q₁ to Q₆ are the same as defined in the descriptions of Formula 1.

19. The amine compound of claim 16,
wherein, the amine compound represented by Formula 1 is represented by any one selected from among Formulas 3-1 to 3-4: 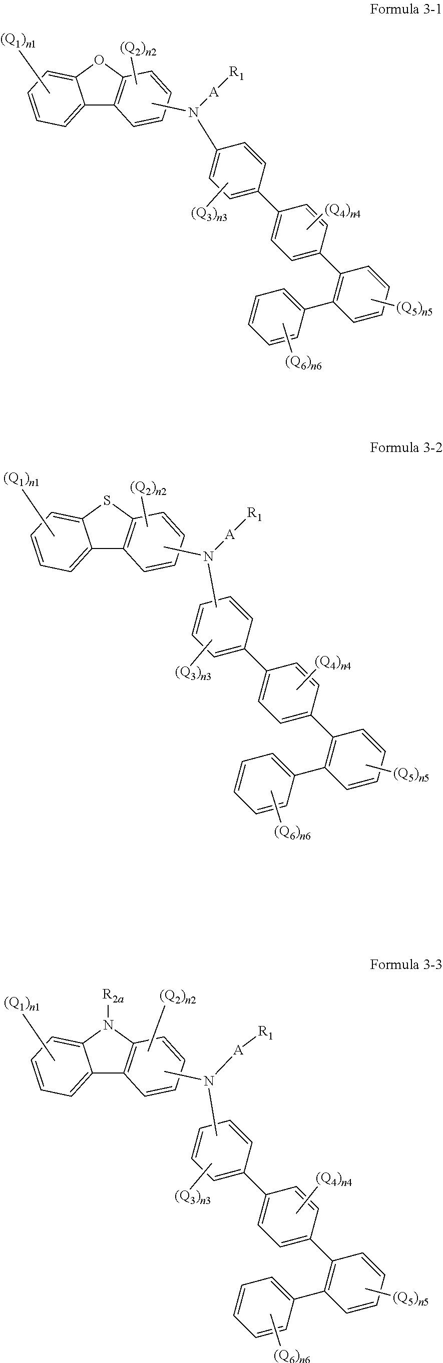 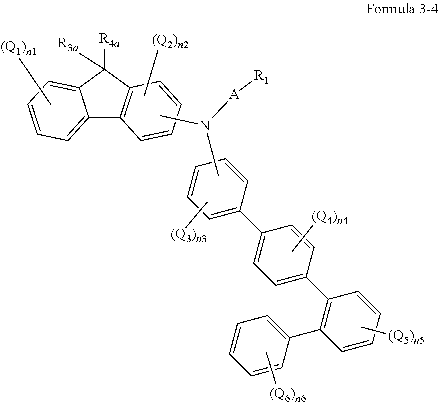
wherein, in Formulas 3-3 and 3-4,
R₂ₐ, R₃ₐ, and R₄ₐ are each independently a hydrogen atom, a deuterium atom,
an unsubstituted alkyl group having 1 to 30 carbon atoms, or an unsubstituted aryl group having 6 to 30 ring-forming carbon atoms, and
n1 to n6, A, R₁, and Q₁ to Q₆ are the same as defined in the descriptions of Formula 1.

20. The amine compound of claim 16,
wherein the amine compound represented by Formula 1 is represented by any one selected from among compounds in Compound Group 1: 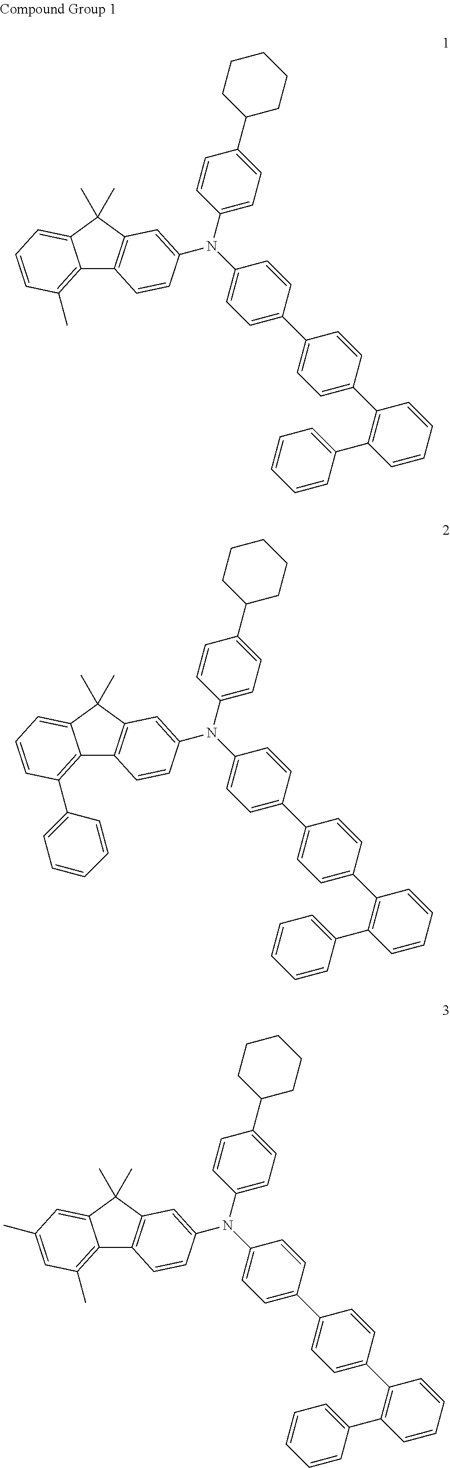 [SMILES: Cc1cccc2c1-c1ccc(N(c3ccc(-c4ccc(-c5ccccc5-c5ccccc5)cc4)cc3)c3ccc(C4CCCCC4)cc3)cc1C2(C)C ; CC1(C)c2cc(N(c3ccc(-c4ccc(-c5ccccc5-c5ccccc5)cc4)cc3)c3ccc(C4CCCCC4)cc3)ccc2-c2c(-c3ccccc3)cccc21 ; Cc1cc(C)c2c(c1)C(C)(C)c1cc(N(c3ccc(-c4ccc(-c5ccccc5-c5ccccc5)cc4)cc3)c3ccc(C4CCCCC4)cc3)ccc1-2] 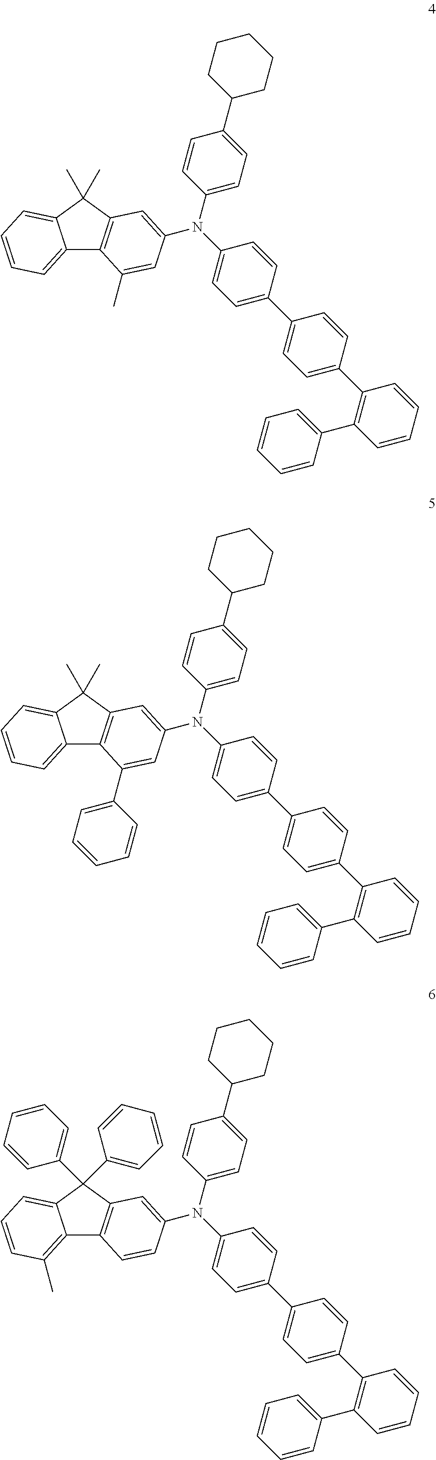 [SMILES: Cc1cc(N(c2ccc(-c3ccc(-c4ccccc4-c4ccccc4)cc3)cc2)c2ccc(C3CCCCC3)cc2)cc2c1-c1ccccc1C2(C)C ; CC1(C)c2ccccc2-c2c(-c3ccccc3)cc(N(c3ccc(-c4ccc(-c5ccccc5-c5ccccc5)cc4)cc3)c3ccc(C4CCCCC4)cc3)cc21 ; Cc1cccc2c1-c1ccc(N(c3ccc(-c4ccc(-c5ccccc5-c5ccccc5)cc4)cc3)c3ccc(C4CCCCC4)cc3)cc1C2(c1ccccc1)c1ccccc1] 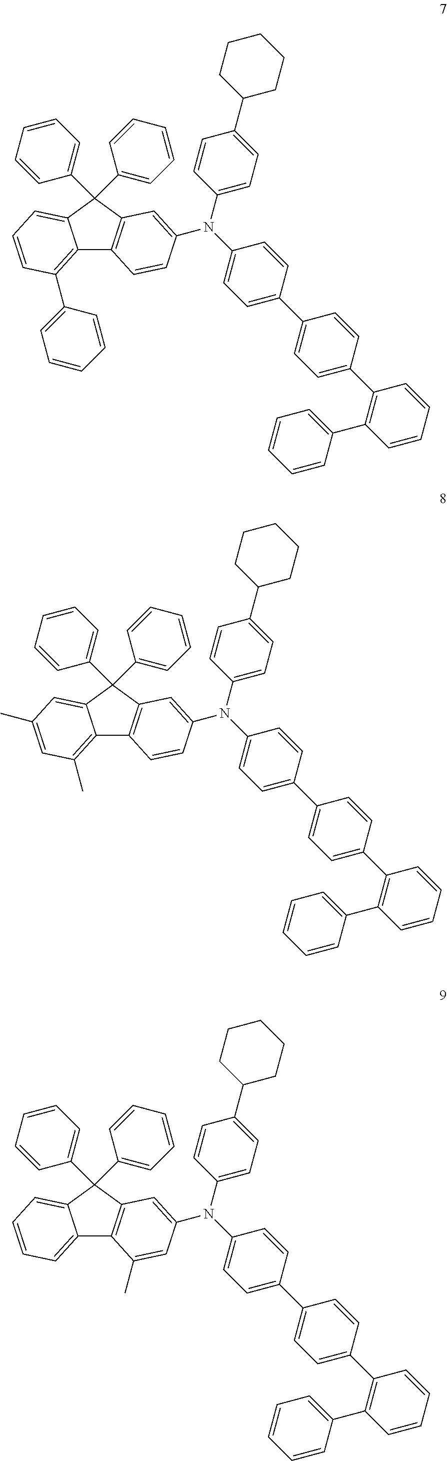 [SMILES: c1ccc(-c2ccccc2-c2ccc(-c3ccc(N(c4ccc(C5CCCCC5)cc4)c4ccc5c(c4)C(c4ccccc4)(c4ccccc4)c4cccc(-c6ccccc6)c4-5)cc3)cc2)cc1 ; Cc1cc(C)c2c(c1)C(c1ccccc1)(c1ccccc1)c1cc(N(c3ccc(-c4ccc(-c5ccccc5-c5ccccc5)cc4)cc3)c3ccc(C4CCCCC4)cc3)ccc1-2 ; Cc1cc(N(c2ccc(-c3ccc(-c4ccccc4-c4ccccc4)cc3)cc2)c2ccc(C3CCCCC3)cc2)cc2c1-c1ccccc1C2(c1ccccc1)c1ccccc1] 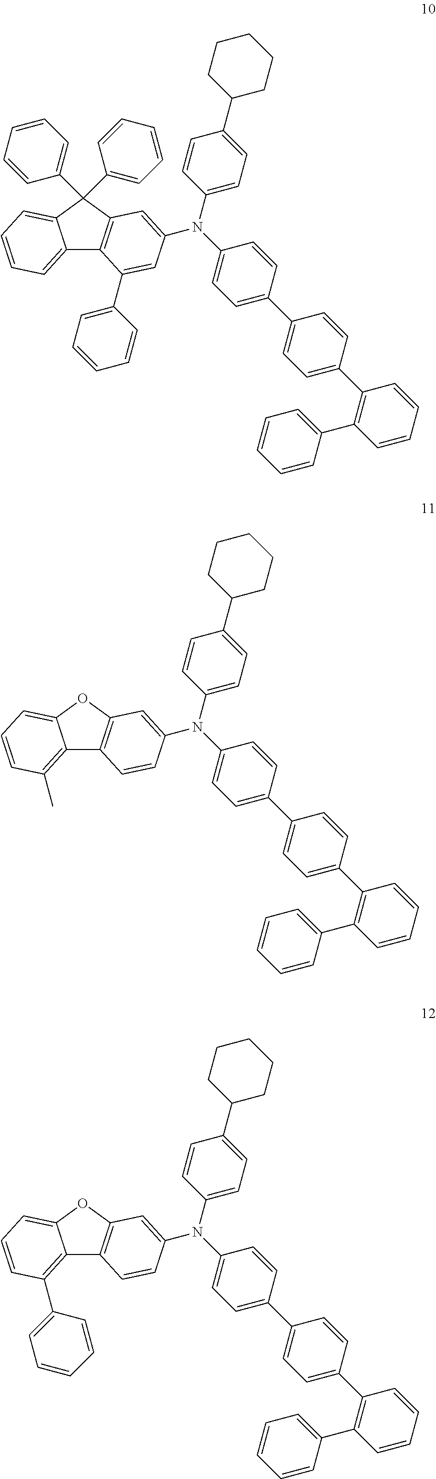 [SMILES: c1ccc(-c2ccccc2-c2ccc(-c3ccc(N(c4ccc(C5CCCCC5)cc4)c4cc(-c5ccccc5)c5c(c4)C(c4ccccc4)(c4ccccc4)c4ccccc4-5)cc3)cc2)cc1 ; Cc1cccc2oc3cc(N(c4ccc(-c5ccc(-c6ccccc6-c6ccccc6)cc5)cc4)c4ccc(C5CCCCC5)cc4)ccc3c12 ; c1ccc(-c2ccccc2-c2ccc(-c3ccc(N(c4ccc(C5CCCCC5)cc4)c4ccc5c(c4)oc4cccc(-c6ccccc6)c45)cc3)cc2)cc1] 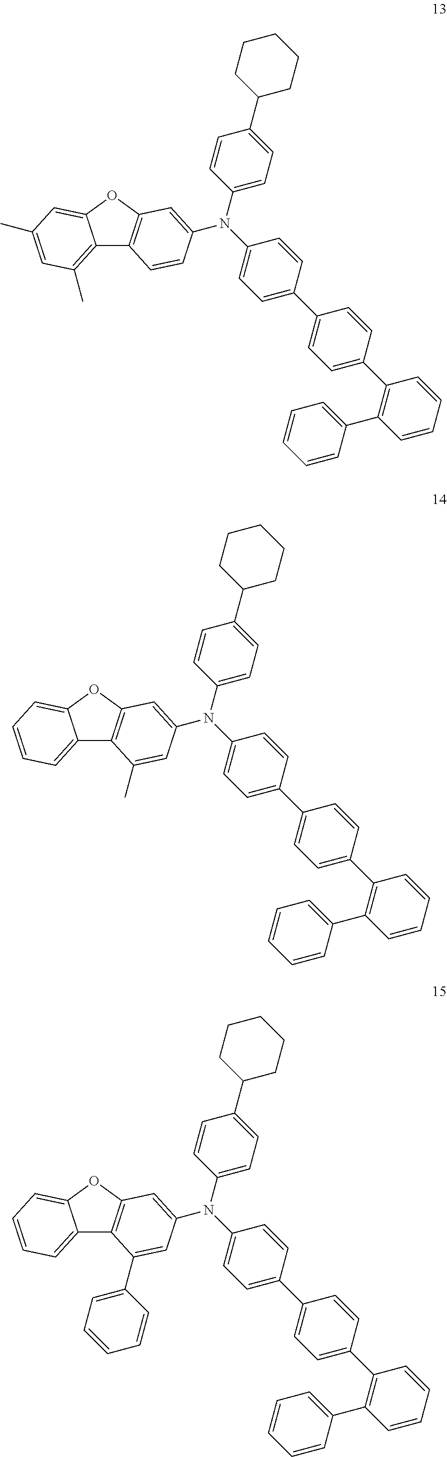 [SMILES: Cc1cc(C)c2c(c1)oc1cc(N(c3ccc(-c4ccc(-c5ccccc5-c5ccccc5)cc4)cc3)c3ccc(C4CCCCC4)cc3)ccc12 ; Cc1cc(N(c2ccc(-c3ccc(-c4ccccc4-c4ccccc4)cc3)cc2)c2ccc(C3CCCCC3)cc2)cc2oc3ccccc3c12 ; c1ccc(-c2ccccc2-c2ccc(-c3ccc(N(c4ccc(C5CCCCC5)cc4)c4cc(-c5ccccc5)c5c(c4)oc4ccccc45)cc3)cc2)cc1] 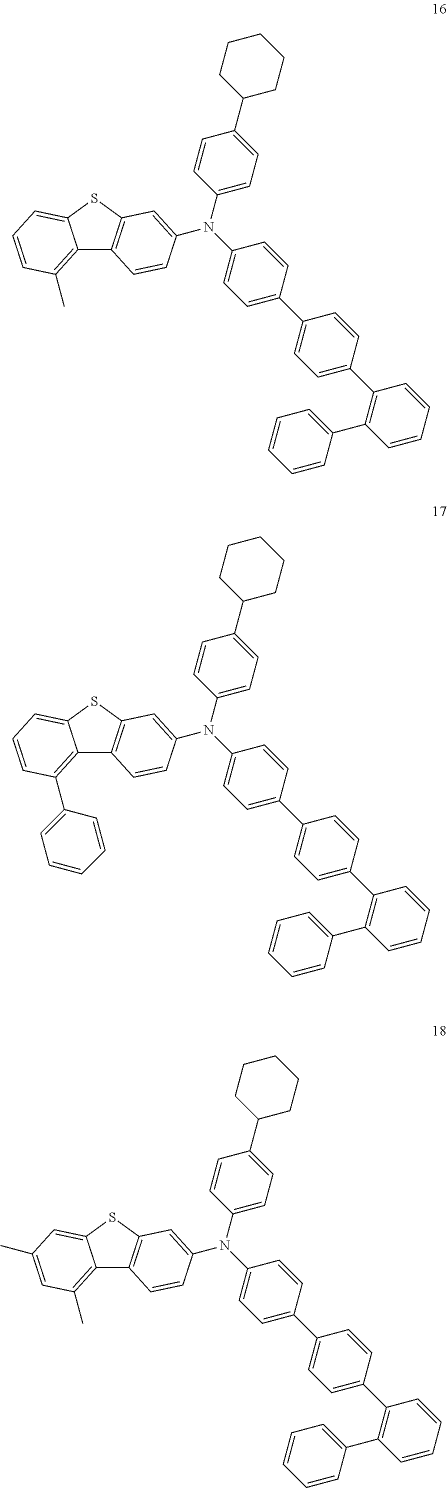 [SMILES: Cc1cccc2sc3cc(N(c4ccc(-c5ccc(-c6ccccc6-c6ccccc6)cc5)cc4)c4ccc(C5CCCCC5)cc4)ccc3c12 ; c1ccc(-c2ccccc2-c2ccc(-c3ccc(N(c4ccc(C5CCCCC5)cc4)c4ccc5c(c4)sc4cccc(-c6ccccc6)c45)cc3)cc2)cc1 ; Cc1cc(C)c2c(c1)sc1cc(N(c3ccc(-c4ccc(-c5ccccc5-c5ccccc5)cc4)cc3)c3ccc(C4CCCCC4)cc3)ccc12] 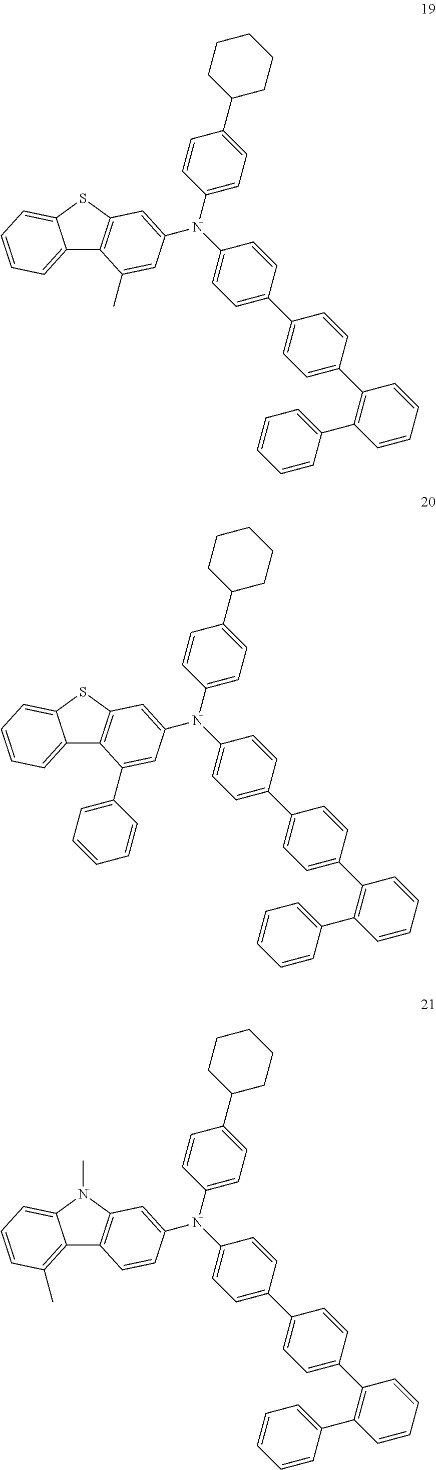 [SMILES: Cc1cc(N(c2ccc(-c3ccc(-c4ccccc4-c4ccccc4)cc3)cc2)c2ccc(C3CCCCC3)cc2)cc2sc3ccccc3c12 ; c1ccc(-c2ccccc2-c2ccc(-c3ccc(N(c4ccc(C5CCCCC5)cc4)c4cc(-c5ccccc5)c5c(c4)sc4ccccc45)cc3)cc2)cc1 ; Cc1cccc2c1c1ccc(N(c3ccc(-c4ccc(-c5ccccc5-c5ccccc5)cc4)cc3)c3ccc(C4CCCCC4)cc3)cc1n2C] 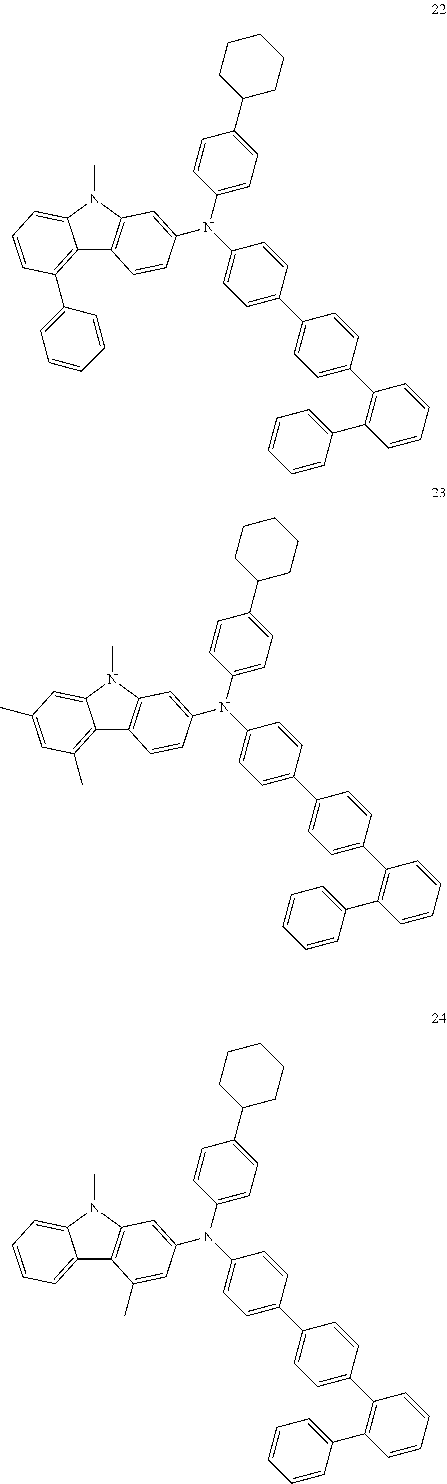 [SMILES: Cn1c2cc(N(c3ccc(-c4ccc(-c5ccccc5-c5ccccc5)cc4)cc3)c3ccc(C4CCCCC4)cc3)ccc2c2c(-c3ccccc3)cccc21 ; Cc1cc(C)c2c3ccc(N(c4ccc(-c5ccc(-c6ccccc6-c6ccccc6)cc5)cc4)c4ccc(C5CCCCC5)cc4)cc3n(C)c2c1 ; Cc1cc(N(c2ccc(-c3ccc(-c4ccccc4-c4ccccc4)cc3)cc2)c2ccc(C3CCCCC3)cc2)cc2c1c1ccccc1n2C] 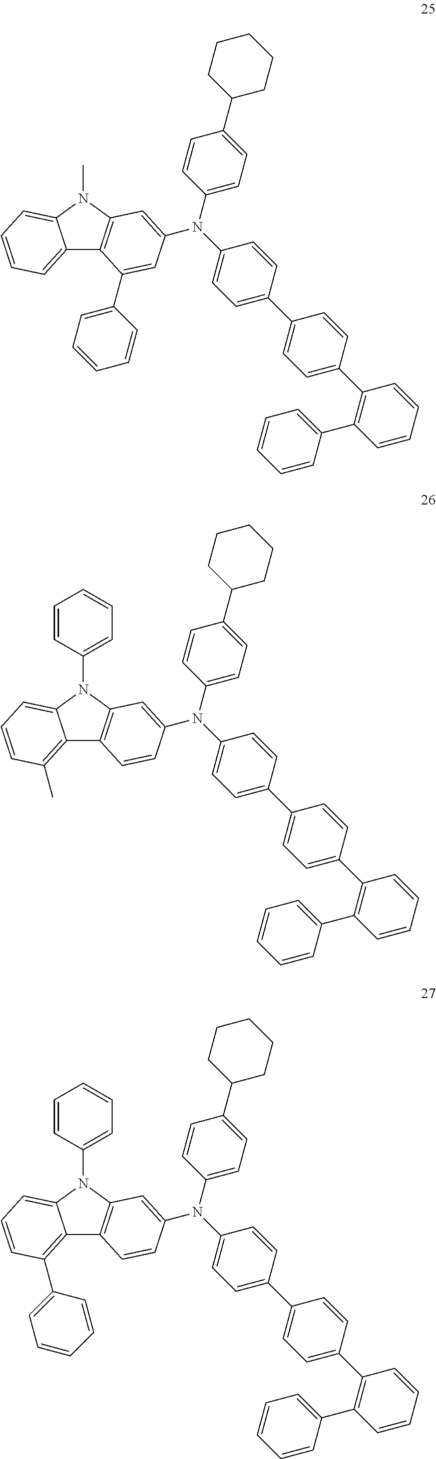 [SMILES: Cn1c2ccccc2c2c(-c3ccccc3)cc(N(c3ccc(-c4ccc(-c5ccccc5-c5ccccc5)cc4)cc3)c3ccc(C4CCCCC4)cc3)cc21 ; Cc1cccc2c1c1ccc(N(c3ccc(-c4ccc(-c5ccccc5-c5ccccc5)cc4)cc3)c3ccc(C4CCCCC4)cc3)cc1n2-c1ccccc1 ; c1ccc(-c2ccccc2-c2ccc(-c3ccc(N(c4ccc(C5CCCCC5)cc4)c4ccc5c6c(-c7ccccc7)cccc6n(-c6ccccc6)c5c4)cc3)cc2)cc1] 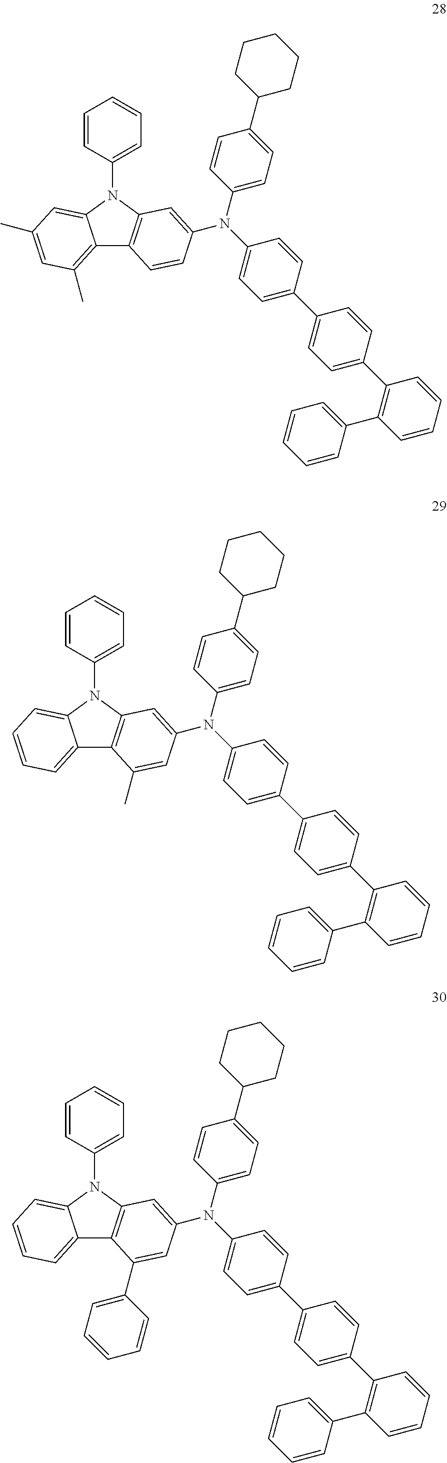 [SMILES: Cc1cc(C)c2c3ccc(N(c4ccc(-c5ccc(-c6ccccc6-c6ccccc6)cc5)cc4)c4ccc(C5CCCCC5)cc4)cc3n(-c3ccccc3)c2c1 ; Cc1cc(N(c2ccc(-c3ccc(-c4ccccc4-c4ccccc4)cc3)cc2)c2ccc(C3CCCCC3)cc2)cc2c1c1ccccc1n2-c1ccccc1 ; c1ccc(-c2ccccc2-c2ccc(-c3ccc(N(c4ccc(C5CCCCC5)cc4)c4cc(-c5ccccc5)c5c6ccccc6n(-c6ccccc6)c5c4)cc3)cc2)cc1] 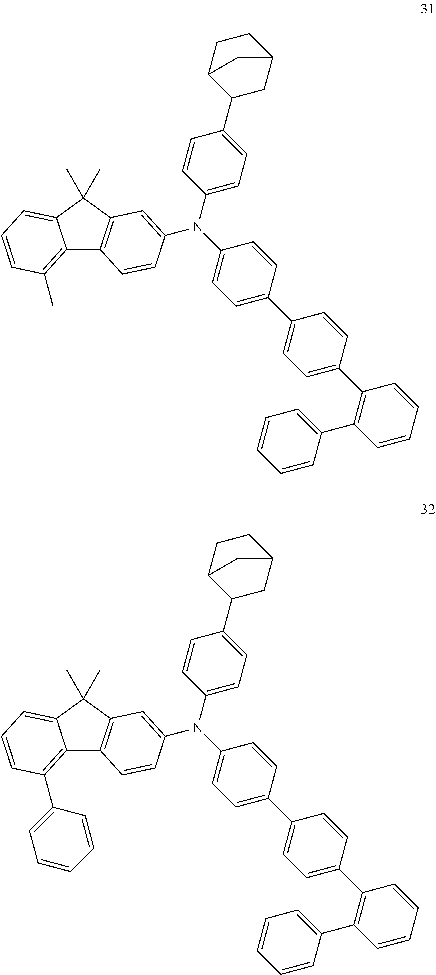 [SMILES: Cc1cccc2c1-c1ccc(N(c3ccc(-c4ccc(-c5ccccc5-c5ccccc5)cc4)cc3)c3ccc(C4CC5CCC4C5)cc3)cc1C2(C)C ; CC1(C)c2cc(N(c3ccc(-c4ccc(-c5ccccc5-c5ccccc5)cc4)cc3)c3ccc(C4CC5CCC4C5)cc3)ccc2-c2c(-c3ccccc3)cccc21] 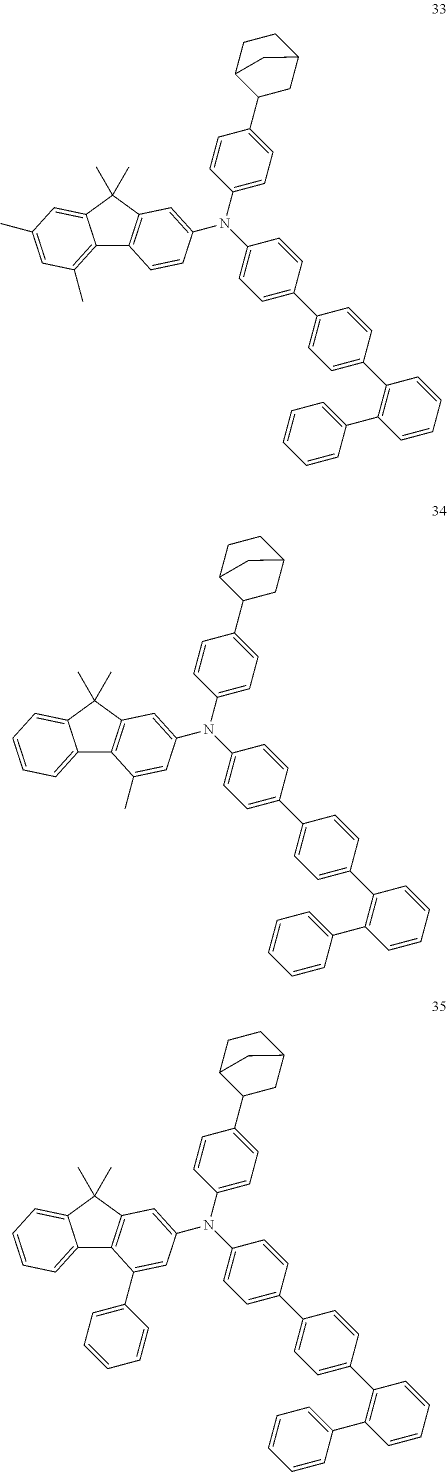 [SMILES: Cc1cc(C)c2c(c1)C(C)(C)c1cc(N(c3ccc(-c4ccc(-c5ccccc5-c5ccccc5)cc4)cc3)c3ccc(C4CC5CCC4C5)cc3)ccc1-2 ; Cc1cc(N(c2ccc(-c3ccc(-c4ccccc4-c4ccccc4)cc3)cc2)c2ccc(C3CC4CCC3C4)cc2)cc2c1-c1ccccc1C2(C)C ; CC1(C)c2ccccc2-c2c(-c3ccccc3)cc(N(c3ccc(-c4ccc(-c5ccccc5-c5ccccc5)cc4)cc3)c3ccc(C4CC5CCC4C5)cc3)cc21] 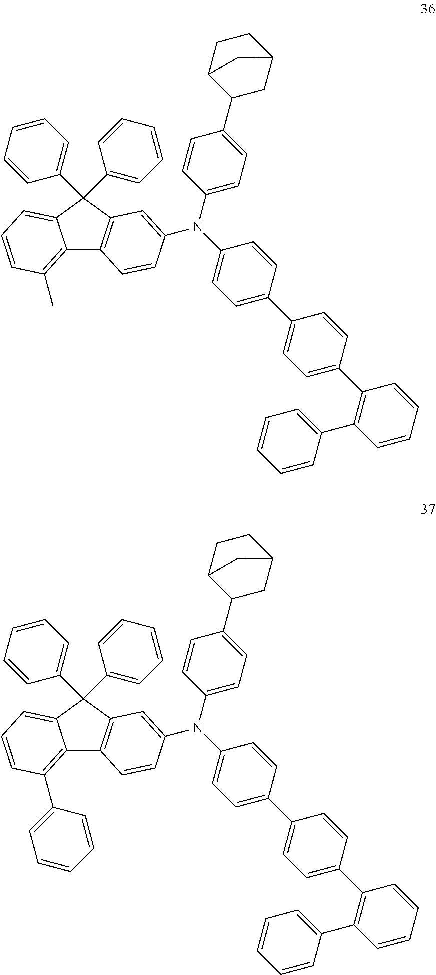 [SMILES: Cc1cccc2c1-c1ccc(N(c3ccc(-c4ccc(-c5ccccc5-c5ccccc5)cc4)cc3)c3ccc(C4CC5CCC4C5)cc3)cc1C2(c1ccccc1)c1ccccc1 ; c1ccc(-c2ccccc2-c2ccc(-c3ccc(N(c4ccc(C5CC6CCC5C6)cc4)c4ccc5c(c4)C(c4ccccc4)(c4ccccc4)c4cccc(-c6ccccc6)c4-5)cc3)cc2)cc1] 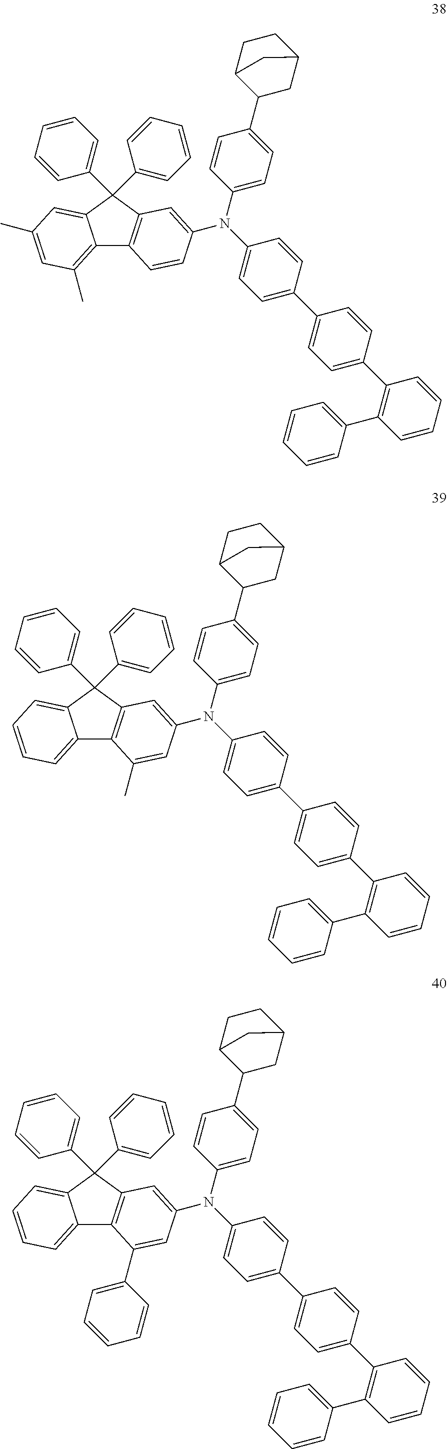 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C4CC5CCC4C5)cc3)ccc1-2 ; Cc1cc(N(c2ccc(-c3ccc(-c4ccccc4-c4ccccc4)cc3)cc2)c2ccc(C3CC4CCC3C4)cc2)cc2c1-c1ccccc1C2(c1ccccc1)c1ccccc1 ; c1ccc(-c2ccccc2-c2ccc(-c3ccc(N(c4ccc(C5CC6CCC5C6)cc4)c4cc(-c5ccccc5)c5c(c4)C(c4ccccc4)(c4ccccc4)c4ccccc4-5)cc3)cc2)cc1] 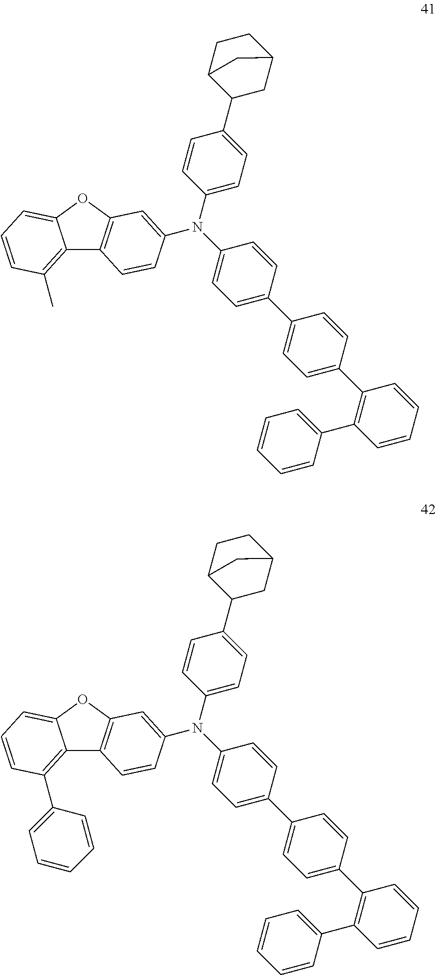 [SMILES: Cc1cccc2oc3cc(N(c4ccc(-c5ccc(-c6ccccc6-c6ccccc6)cc5)cc4)c4ccc(C5CC6CCC5C6)cc4)ccc3c12 ; c1ccc(-c2ccccc2-c2ccc(-c3ccc(N(c4ccc(C5CC6CCC5C6)cc4)c4ccc5c(c4)oc4cccc(-c6ccccc6)c45)cc3)cc2)cc1] 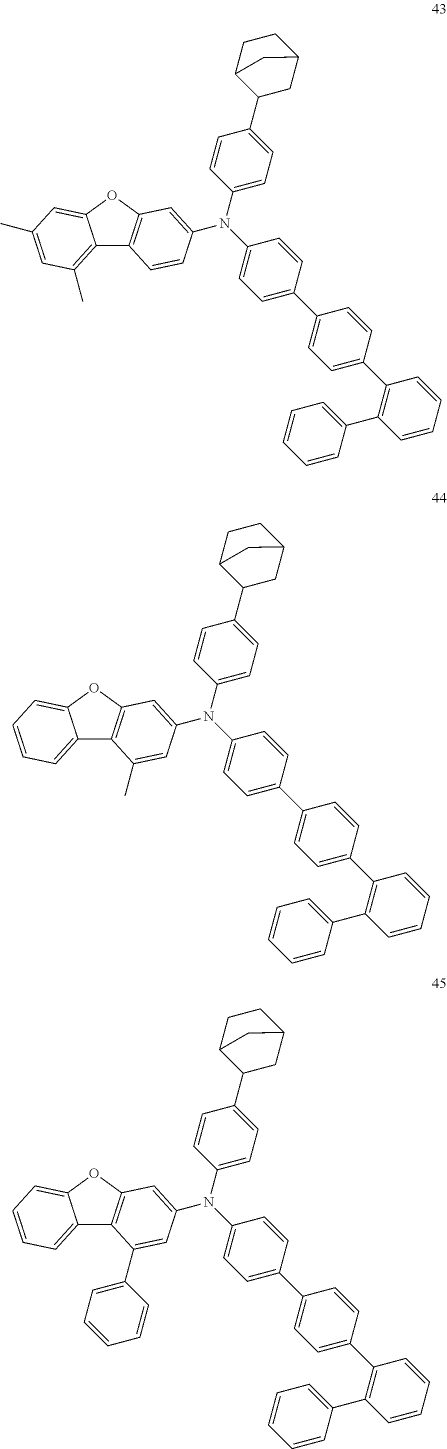 [SMILES: Cc1cc(C)c2c(c1)oc1cc(N(c3ccc(-c4ccc(-c5ccccc5-c5ccccc5)cc4)cc3)c3ccc(C4CC5CCC4C5)cc3)ccc12 ; Cc1cc(N(c2ccc(-c3ccc(-c4ccccc4-c4ccccc4)cc3)cc2)c2ccc(C3CC4CCC3C4)cc2)cc2oc3ccccc3c12 ; c1ccc(-c2ccccc2-c2ccc(-c3ccc(N(c4ccc(C5CC6CCC5C6)cc4)c4cc(-c5ccccc5)c5c(c4)oc4ccccc45)cc3)cc2)cc1] 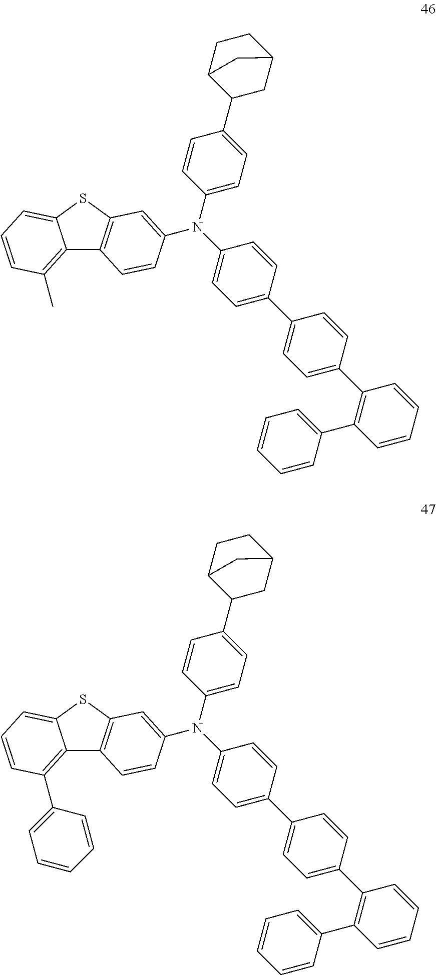 [SMILES: Cc1cccc2sc3cc(N(c4ccc(-c5ccc(-c6ccccc6-c6ccccc6)cc5)cc4)c4ccc(C5CC6CCC5C6)cc4)ccc3c12 ; c1ccc(-c2ccccc2-c2ccc(-c3ccc(N(c4ccc(C5CC6CCC5C6)cc4)c4ccc5c(c4)sc4cccc(-c6ccccc6)c45)cc3)cc2)cc1] 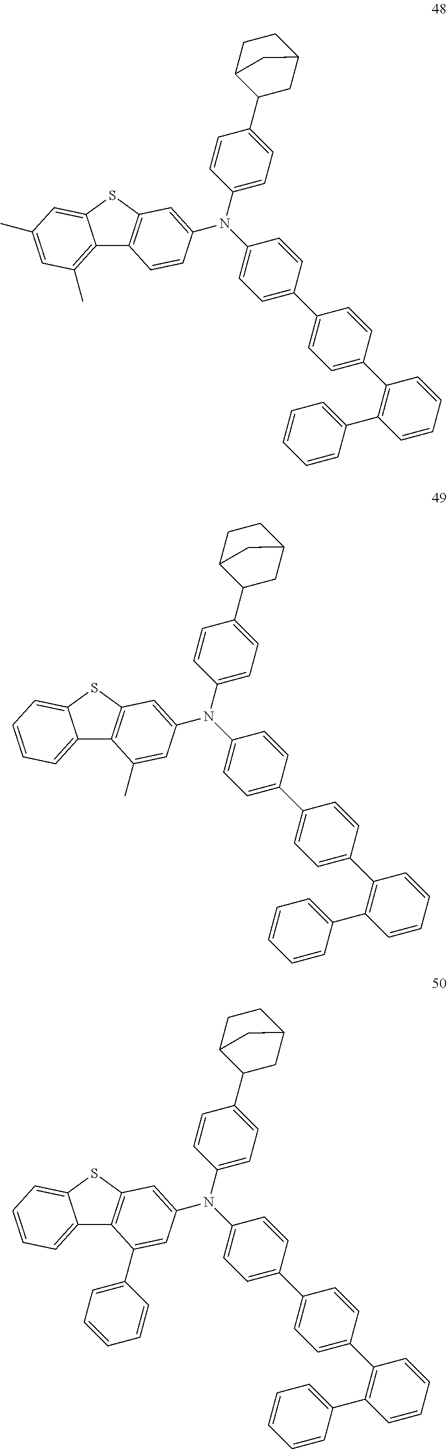 [SMILES: Cc1cc(C)c2c(c1)sc1cc(N(c3ccc(-c4ccc(-c5ccccc5-c5ccccc5)cc4)cc3)c3ccc(C4CC5CCC4C5)cc3)ccc12 ; Cc1cc(N(c2ccc(-c3ccc(-c4ccccc4-c4ccccc4)cc3)cc2)c2ccc(C3CC4CCC3C4)cc2)cc2sc3ccccc3c12 ; c1ccc(-c2ccccc2-c2ccc(-c3ccc(N(c4ccc(C5CC6CCC5C6)cc4)c4cc(-c5ccccc5)c5c(c4)sc4ccccc45)cc3)cc2)cc1] 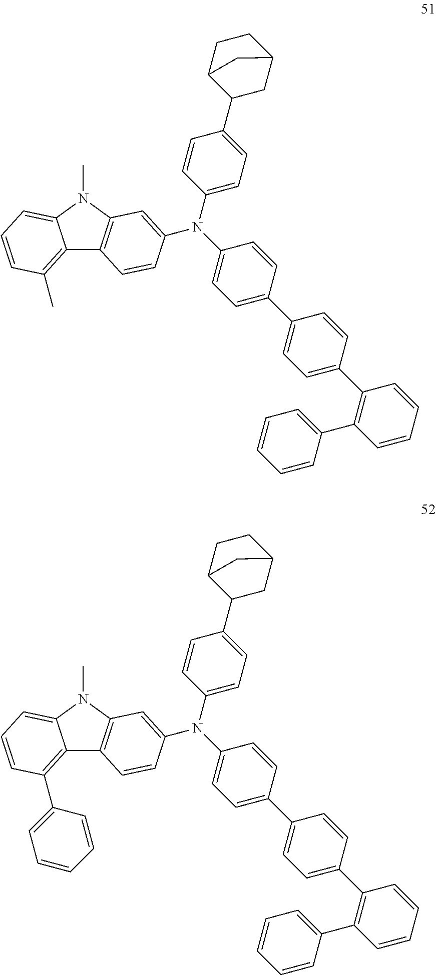 [SMILES: Cc1cccc2c1c1ccc(N(c3ccc(-c4ccc(-c5ccccc5-c5ccccc5)cc4)cc3)c3ccc(C4CC5CCC4C5)cc3)cc1n2C ; Cn1c2cc(N(c3ccc(-c4ccc(-c5ccccc5-c5ccccc5)cc4)cc3)c3ccc(C4CC5CCC4C5)cc3)ccc2c2c(-c3ccccc3)cccc21] 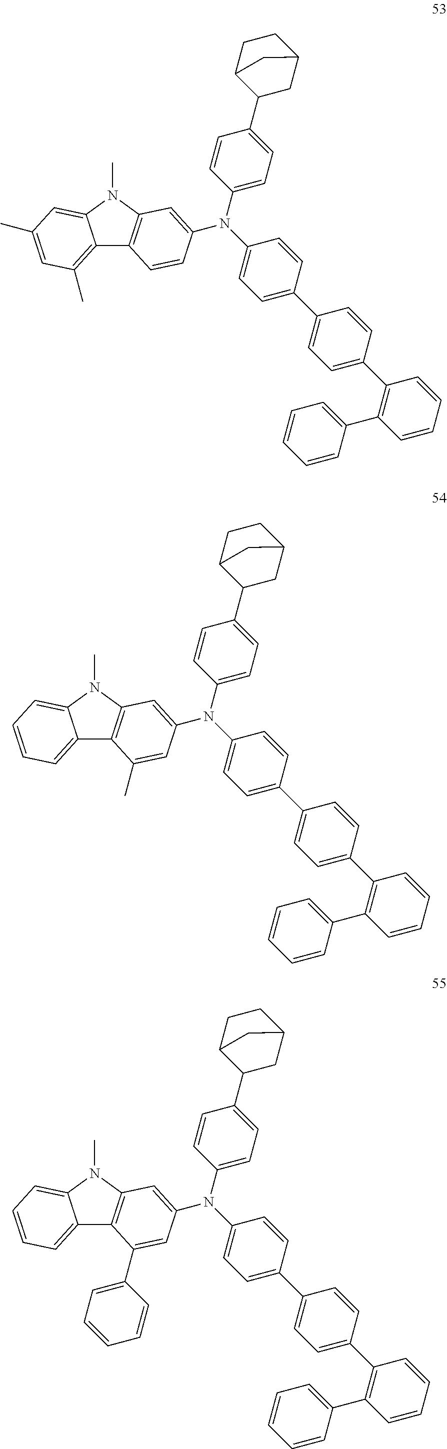 [SMILES: Cc1cc(C)c2c3ccc(N(c4ccc(-c5ccc(-c6ccccc6-c6ccccc6)cc5)cc4)c4ccc(C5CC6CCC5C6)cc4)cc3n(C)c2c1 ; Cc1cc(N(c2ccc(-c3ccc(-c4ccccc4-c4ccccc4)cc3)cc2)c2ccc(C3CC4CCC3C4)cc2)cc2c1c1ccccc1n2C ; Cn1c2ccccc2c2c(-c3ccccc3)cc(N(c3ccc(-c4ccc(-c5ccccc5-c5ccccc5)cc4)cc3)c3ccc(C4CC5CCC4C5)cc3)cc21] 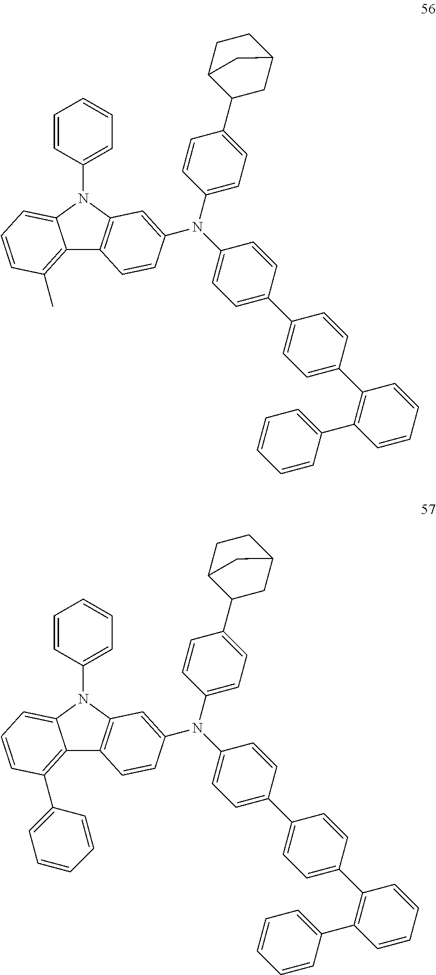 [SMILES: Cc1cccc2c1c1ccc(N(c3ccc(-c4ccc(-c5ccccc5-c5ccccc5)cc4)cc3)c3ccc(C4CC5CCC4C5)cc3)cc1n2-c1ccccc1 ; c1ccc(-c2ccccc2-c2ccc(-c3ccc(N(c4ccc(C5CC6CCC5C6)cc4)c4ccc5c6c(-c7ccccc7)cccc6n(-c6ccccc6)c5c4)cc3)cc2)cc1] 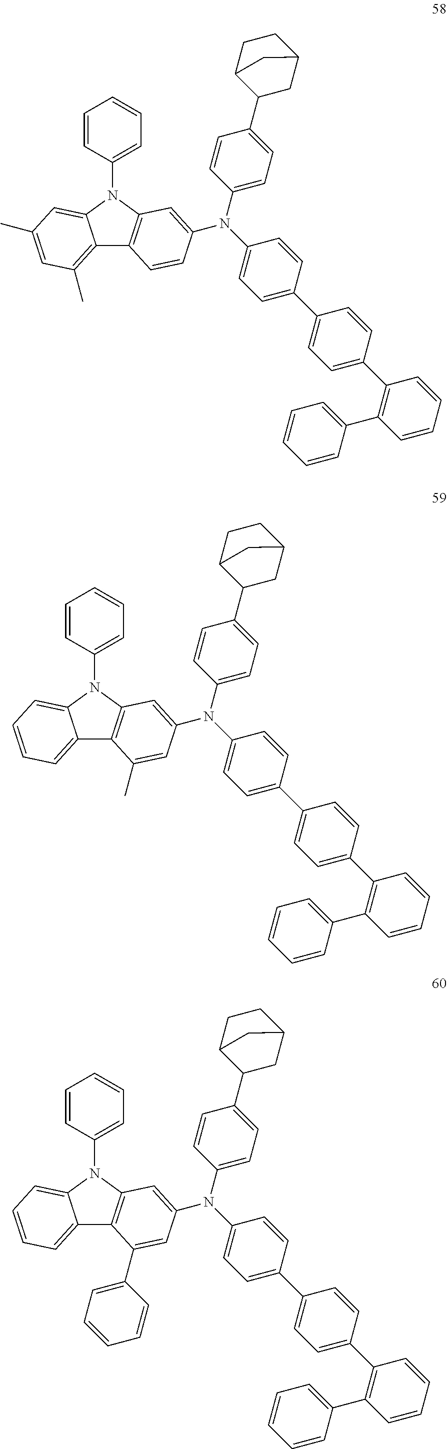 [SMILES: Cc1cc(C)c2c3ccc(N(c4ccc(-c5ccc(-c6ccccc6-c6ccccc6)cc5)cc4)c4ccc(C5CC6CCC5C6)cc4)cc3n(-c3ccccc3)c2c1 ; Cc1cc(N(c2ccc(-c3ccc(-c4ccccc4-c4ccccc4)cc3)cc2)c2ccc(C3CC4CCC3C4)cc2)cc2c1c1ccccc1n2-c1ccccc1 ; c1ccc(-c2ccccc2-c2ccc(-c3ccc(N(c4ccc(C5CC6CCC5C6)cc4)c4cc(-c5ccccc5)c5c6ccccc6n(-c6ccccc6)c5c4)cc3)cc2)cc1] 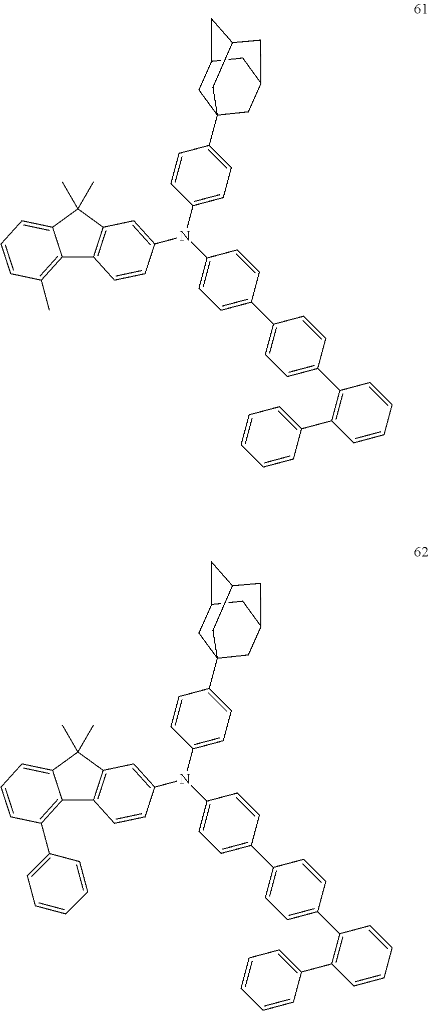 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(C)C ; CC1(C)c2cc(N(c3ccc(-c4ccc(-c5ccccc5-c5ccccc5)cc4)cc3)c3ccc(C45CC6CC(CC(C6)C4)C5)cc3)ccc2-c2c(-c3ccccc3)cccc21] 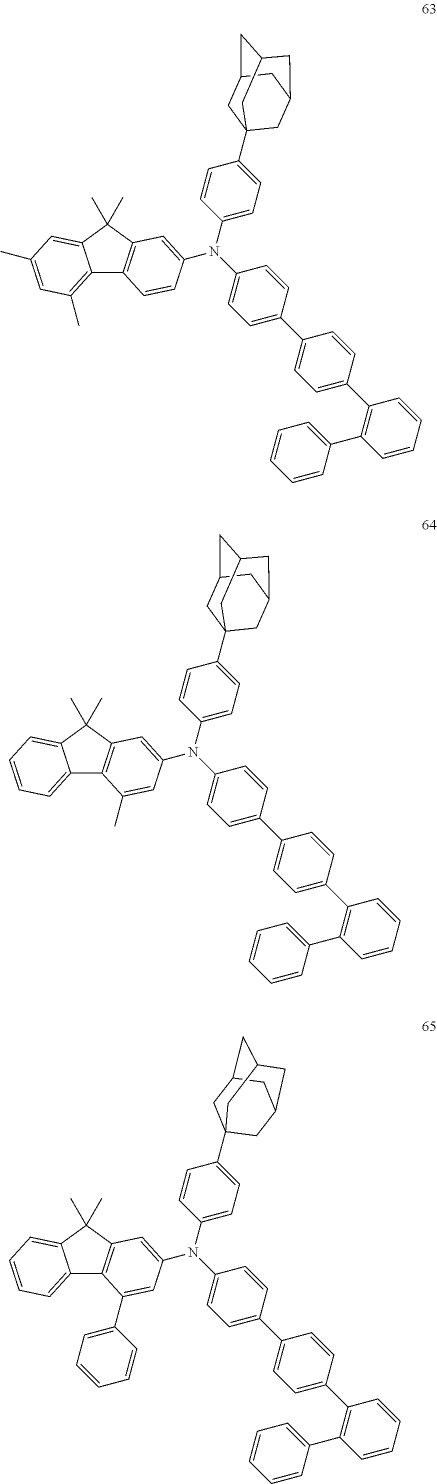 [SMILES: Cc1cc(C)c2c(c1)C(C)(C)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(C)C ; CC1(C)c2ccccc2-c2c(-c3ccccc3)cc(N(c3ccc(-c4ccc(-c5ccccc5-c5ccccc5)cc4)cc3)c3ccc(C45CC6CC(CC(C6)C4)C5)cc3)cc21] 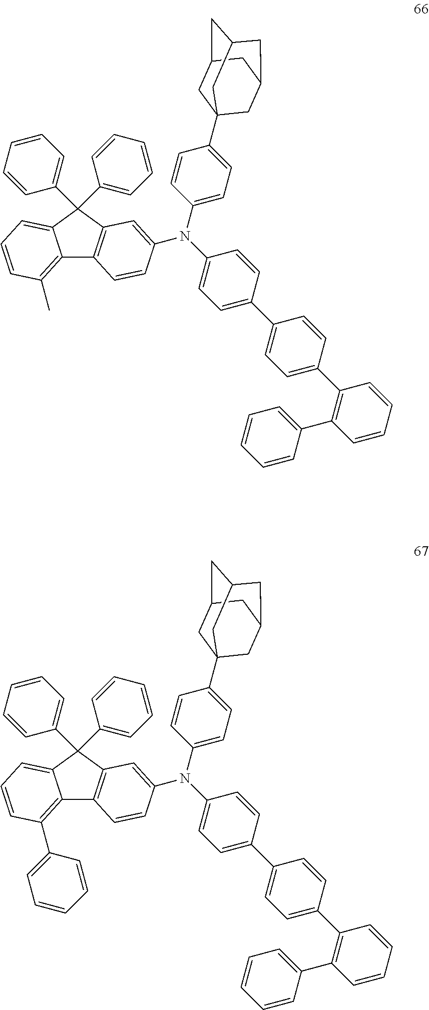 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2(c1ccccc1)c1ccccc1 ; c1ccc(-c2ccccc2-c2ccc(-c3ccc(N(c4ccc(C56CC7CC(CC(C7)C5)C6)cc4)c4ccc5c(c4)C(c4ccccc4)(c4ccccc4)c4cccc(-c6ccccc6)c4-5)cc3)cc2)cc1] 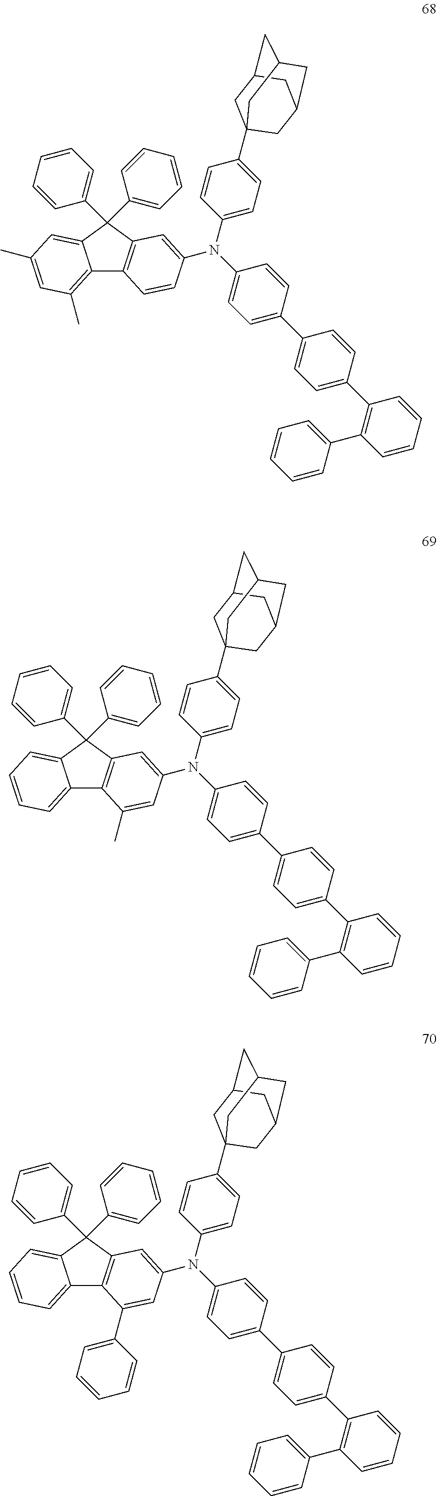 [SMILES: Cc1cc(C)c2c(c1)C(c1ccccc1)(c1ccccc1)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2(c1ccccc1)c1ccccc1 ; c1ccc(-c2ccccc2-c2ccc(-c3ccc(N(c4ccc(C56CC7CC(CC(C7)C5)C6)cc4)c4cc(-c5ccccc5)c5c(c4)C(c4ccccc4)(c4ccccc4)c4ccccc4-5)cc3)cc2)cc1] 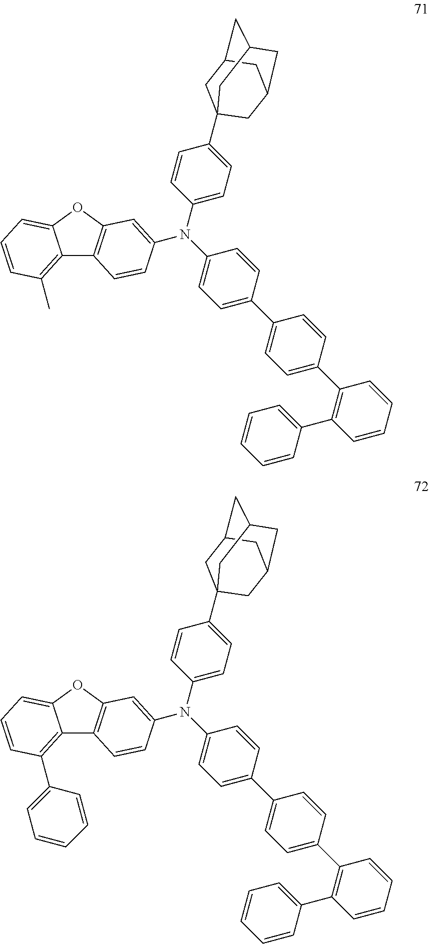 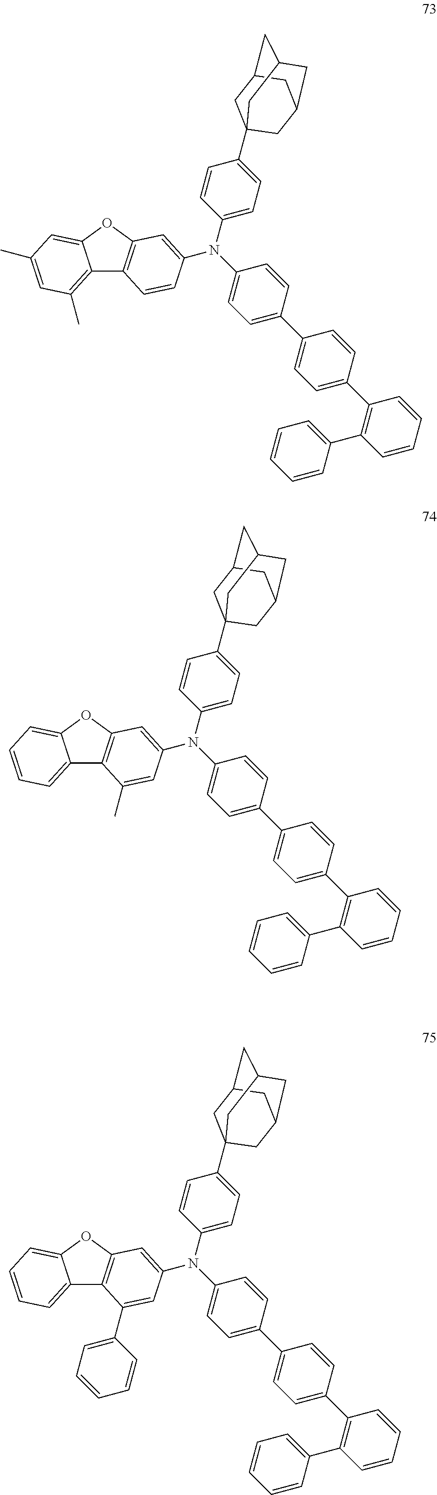 [SMILES: Cc1cc(C)c2c(c1)oc1cc(N(c3ccc(-c4ccc(-c5ccccc5-c5ccccc5)cc4)cc3)c3ccc(C45CC6CC(CC(C6)C4)C5)cc3)ccc12 ; Cc1cc(N(c2ccc(-c3ccc(-c4ccccc4-c4ccccc4)cc3)cc2)c2ccc(C34CC5CC(CC(C5)C3)C4)cc2)cc2oc3ccccc3c12 ; c1ccc(-c2ccccc2-c2ccc(-c3ccc(N(c4ccc(C56CC7CC(CC(C7)C5)C6)cc4)c4cc(-c5ccccc5)c5c(c4)oc4ccccc45)cc3)cc2)cc1] 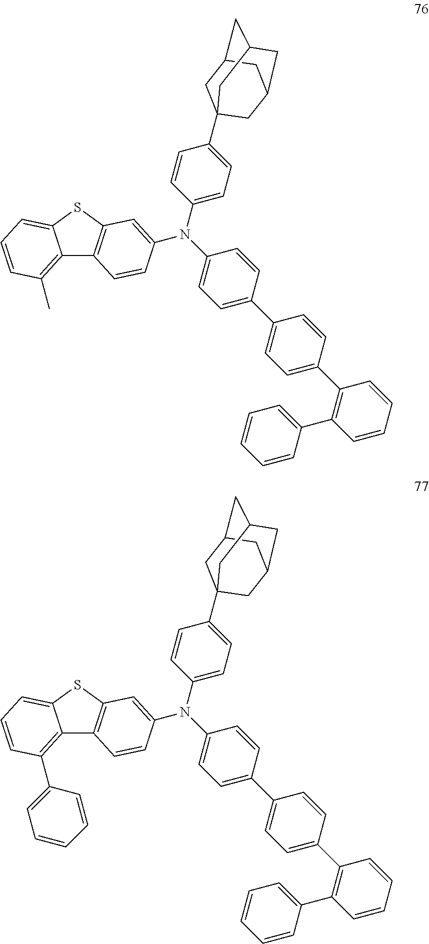 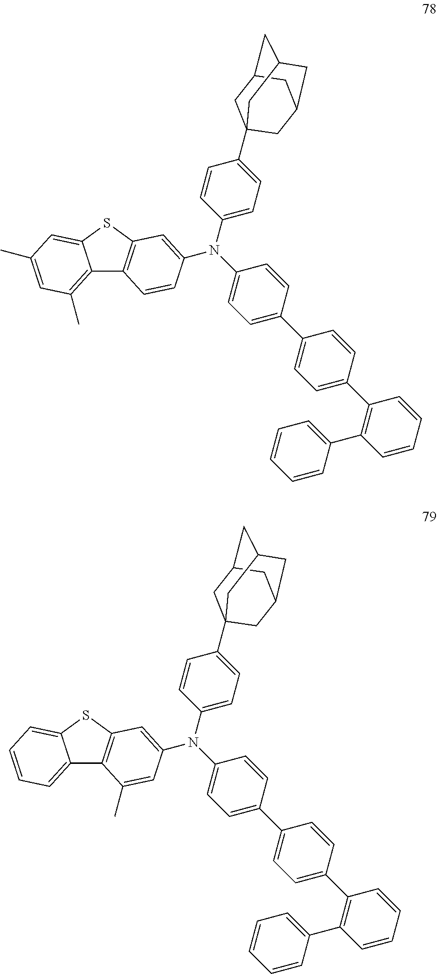 [SMILES: Cc1cc(C)c2c(c1)sc1cc(N(c3ccc(-c4ccc(-c5ccccc5-c5ccccc5)cc4)cc3)c3ccc(C45CC6CC(CC(C6)C4)C5)cc3)ccc12 ; Cc1cc(N(c2ccc(-c3ccc(-c4ccccc4-c4ccccc4)cc3)cc2)c2ccc(C34CC5CC(CC(C5)C3)C4)cc2)cc2sc3ccccc3c12] 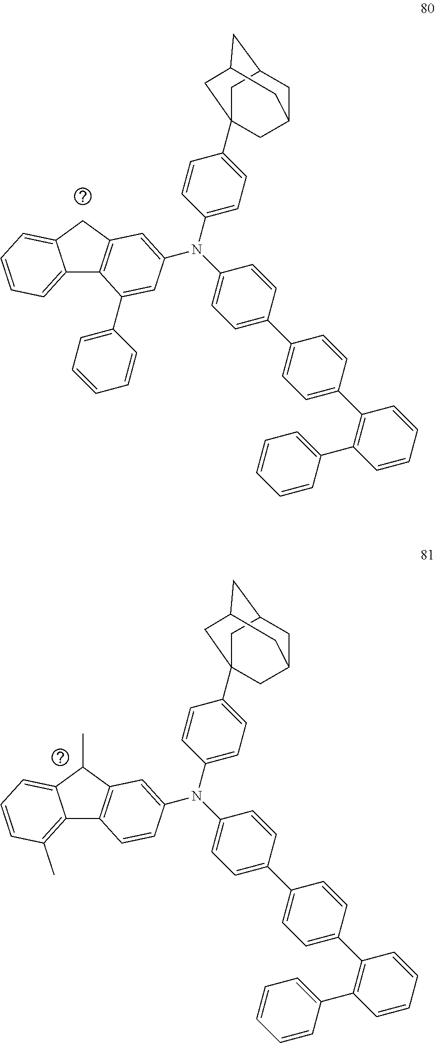 [SMILES: c1ccc(-c2ccccc2-c2ccc(-c3ccc(N(c4ccc(C56CC7CC(CC(C7)C5)C6)cc4)c4cc5c(c(-c6ccccc6)c4)-c4ccccc4C5)cc3)cc2)cc1 ; Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2C] 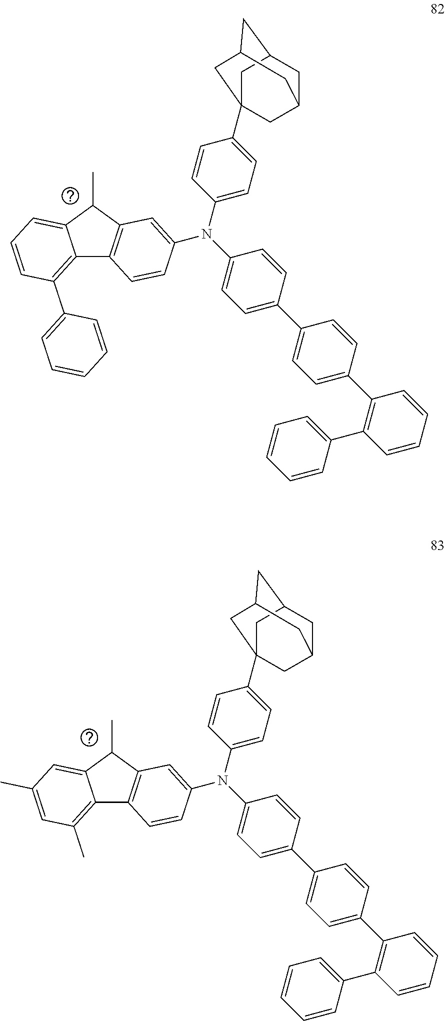 [SMILES: CC1c2cc(N(c3ccc(-c4ccc(-c5ccccc5-c5ccccc5)cc4)cc3)c3ccc(C45CC6CC(CC(C6)C4)C5)cc3)ccc2-c2c(-c3ccccc3)cccc21 ; Cc1cc(C)c2c(c1)C(C)c1cc(N(c3ccc(-c4ccc(-c5ccccc5-c5ccccc5)cc4)cc3)c3ccc(C45CC6CC(CC(C6)C4)C5)cc3)ccc1-2] 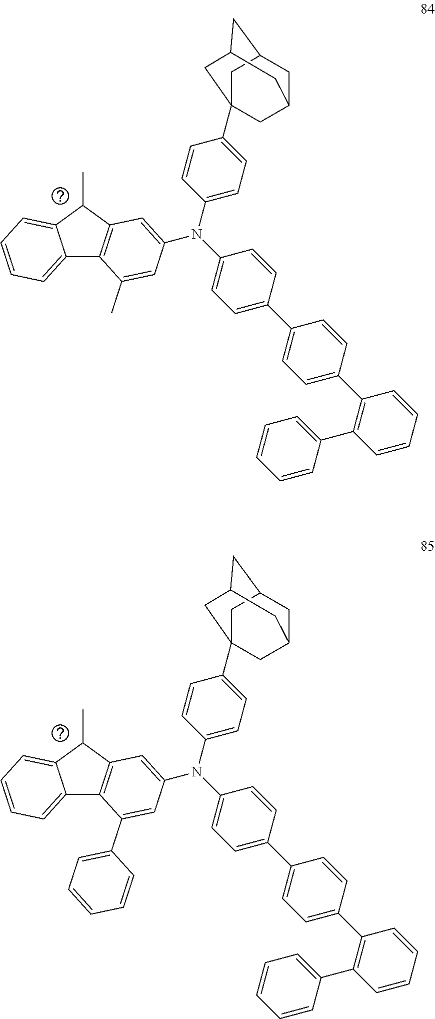 [SMILES: Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2C ; CC1c2ccccc2-c2c(-c3ccccc3)cc(N(c3ccc(-c4ccc(-c5ccccc5-c5ccccc5)cc4)cc3)c3ccc(C45CC6CC(CC(C6)C4)C5)cc3)cc21] 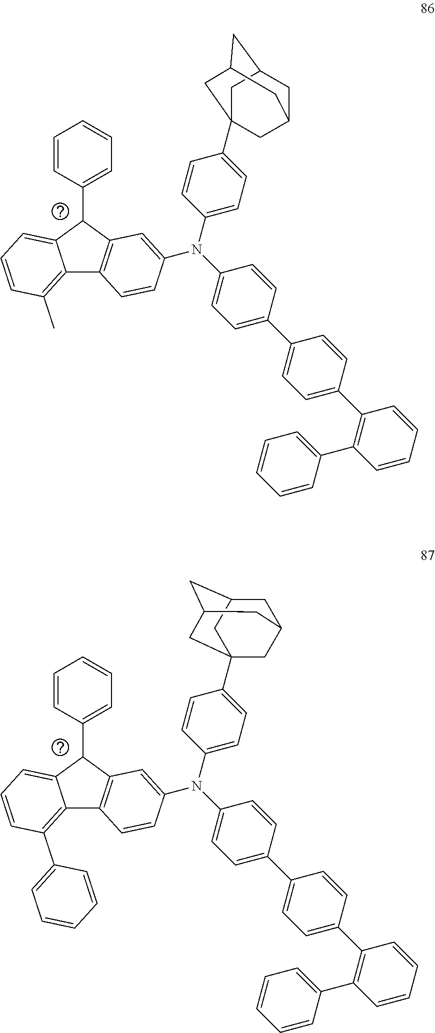 [SMILES: Cc1cccc2c1-c1ccc(N(c3ccc(-c4ccc(-c5ccccc5-c5ccccc5)cc4)cc3)c3ccc(C45CC6CC(CC(C6)C4)C5)cc3)cc1C2c1ccccc1 ; c1ccc(-c2ccccc2-c2ccc(-c3ccc(N(c4ccc(C56CC7CC(CC(C7)C5)C6)cc4)c4ccc5c(c4)C(c4ccccc4)c4cccc(-c6ccccc6)c4-5)cc3)cc2)cc1] 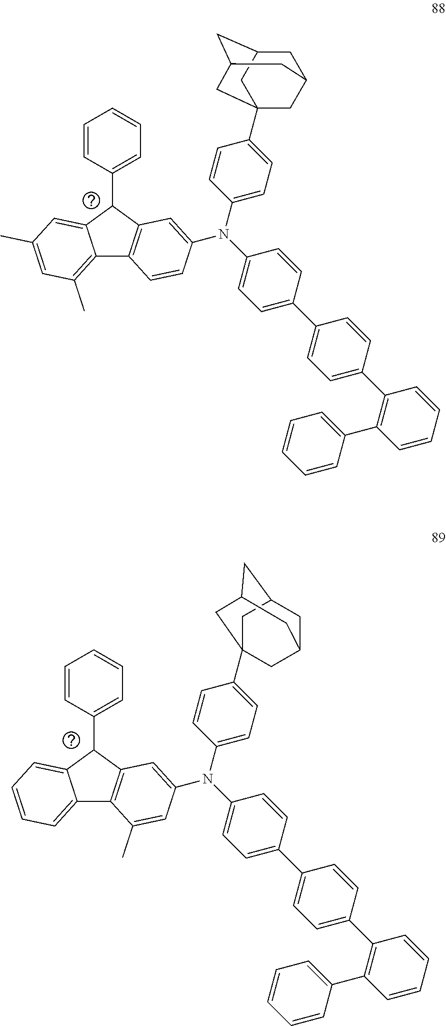 [SMILES: Cc1cc(C)c2c(c1)C(c1ccccc1)c1cc(N(c3ccc(-c4ccc(-c5ccccc5-c5ccccc5)cc4)cc3)c3ccc(C45CC6CC(CC(C6)C4)C5)cc3)ccc1-2 ; Cc1cc(N(c2ccc(-c3ccc(-c4ccccc4-c4ccccc4)cc3)cc2)c2ccc(C34CC5CC(CC(C5)C3)C4)cc2)cc2c1-c1ccccc1C2c1ccccc1] 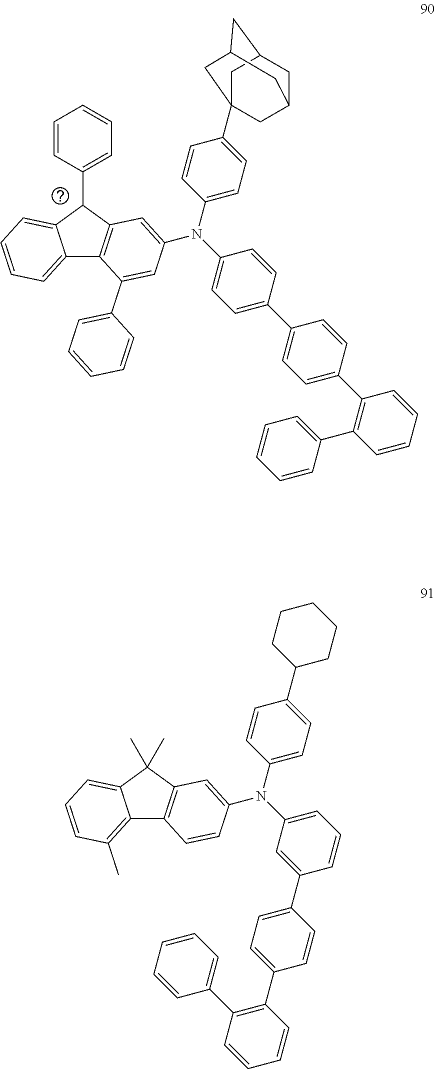 [SMILES: c1ccc(-c2ccccc2-c2ccc(-c3ccc(N(c4ccc(C56CC7CC(CC(C7)C5)C6)cc4)c4cc(-c5ccccc5)c5c(c4)C(c4ccccc4)c4ccccc4-5)cc3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2(C)C] 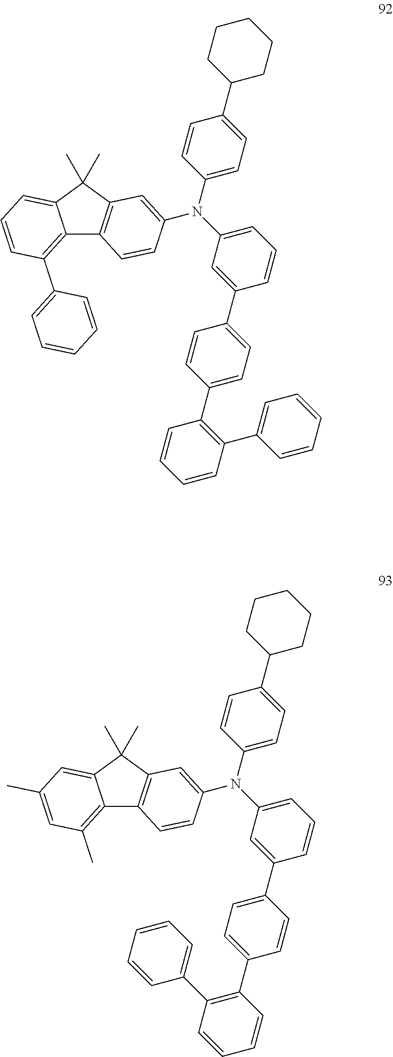 [SMILES: CC1(C)c2cc(N(c3ccc(C4CCCCC4)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C4CCCCC4)cc3)c3cccc(-c4ccc(-c5ccccc5-c5ccccc5)cc4)c3)ccc1-2] 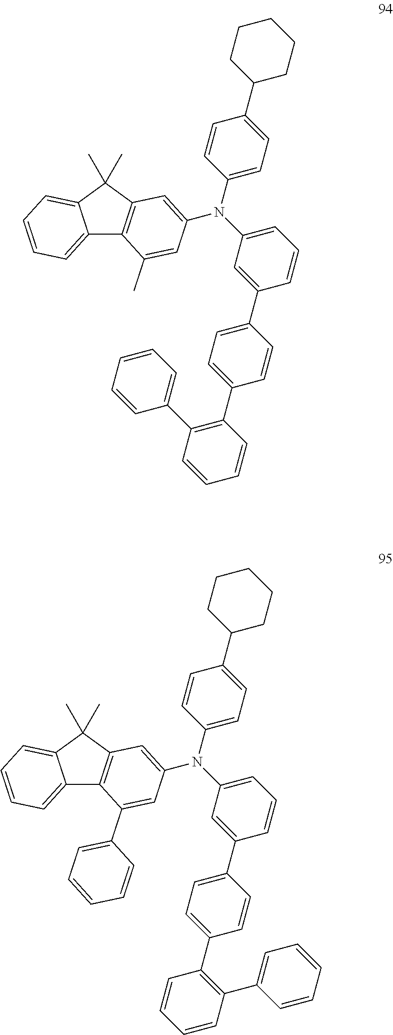 [SMILES: Cc1cc(N(c2ccc(C3CCCCC3)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CCCCC4)cc3)c3cccc(-c4ccc(-c5ccccc5-c5ccccc5)cc4)c3)cc21] 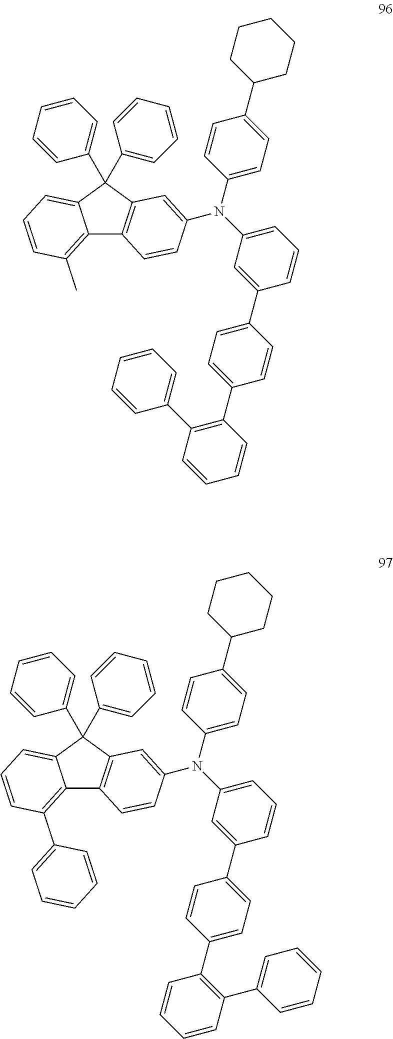 [SMILES: Cc1cccc2c1-c1ccc(N(c3ccc(C4CCCCC4)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CCCCC5)cc4)c4ccc5c(c4)C(c4ccccc4)(c4ccccc4)c4cccc(-c6ccccc6)c4-5)c3)cc2)cc1] 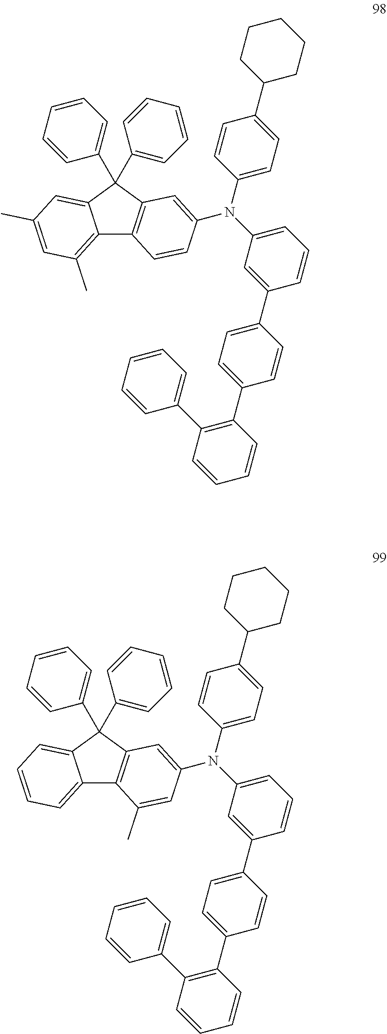 [SMILES: Cc1cc(C)c2c(c1)C(c1ccccc1)(c1ccccc1)c1cc(N(c3ccc(C4CCCCC4)cc3)c3cccc(-c4ccc(-c5ccccc5-c5ccccc5)cc4)c3)ccc1-2 ; Cc1cc(N(c2ccc(C3CCCCC3)cc2)c2cccc(-c3ccc(-c4ccccc4-c4ccccc4)cc3)c2)cc2c1-c1ccccc1C2(c1ccccc1)c1ccccc1] 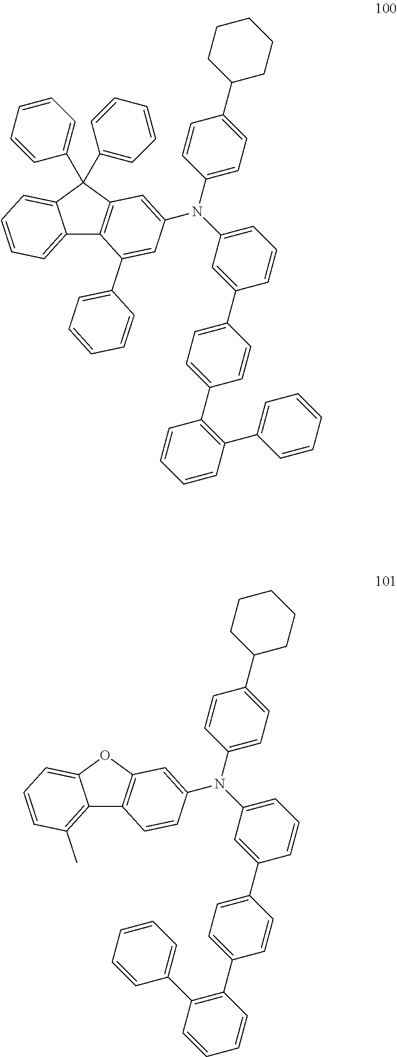 [SMILES: c1ccc(-c2ccccc2-c2ccc(-c3cccc(N(c4ccc(C5CCCCC5)cc4)c4cc(-c5ccccc5)c5c(c4)C(c4ccccc4)(c4ccccc4)c4ccccc4-5)c3)cc2)cc1 ; Cc1cccc2oc3cc(N(c4ccc(C5CCCCC5)cc4)c4cccc(-c5ccc(-c6ccccc6-c6ccccc6)cc5)c4)ccc3c12] 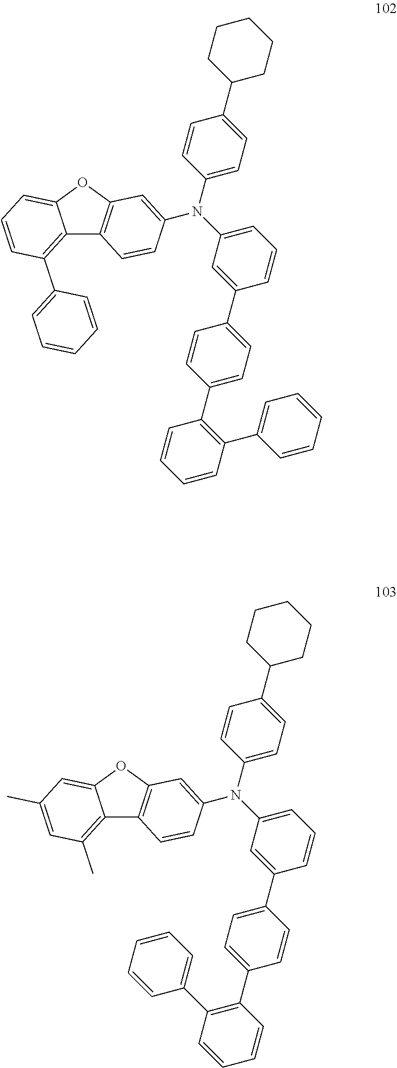 [SMILES: c1ccc(-c2ccccc2-c2ccc(-c3cccc(N(c4ccc(C5CCCCC5)cc4)c4ccc5c(c4)oc4cccc(-c6ccccc6)c45)c3)cc2)cc1 ; Cc1cc(C)c2c(c1)oc1cc(N(c3ccc(C4CCCCC4)cc3)c3cccc(-c4ccc(-c5ccccc5-c5ccccc5)cc4)c3)ccc12] 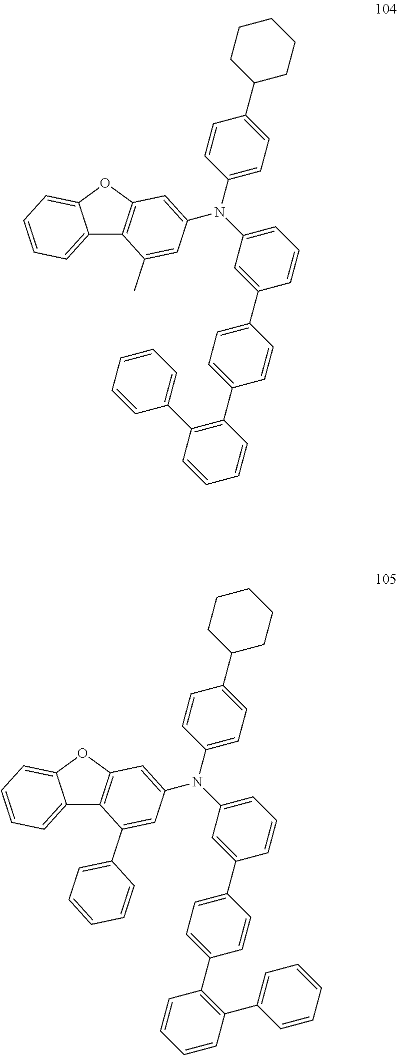 [SMILES: Cc1cc(N(c2ccc(C3CCCCC3)cc2)c2cccc(-c3ccc(-c4ccccc4-c4ccccc4)cc3)c2)cc2oc3ccccc3c12 ; c1ccc(-c2ccccc2-c2ccc(-c3cccc(N(c4ccc(C5CCCCC5)cc4)c4cc(-c5ccccc5)c5c(c4)oc4ccccc45)c3)cc2)cc1] 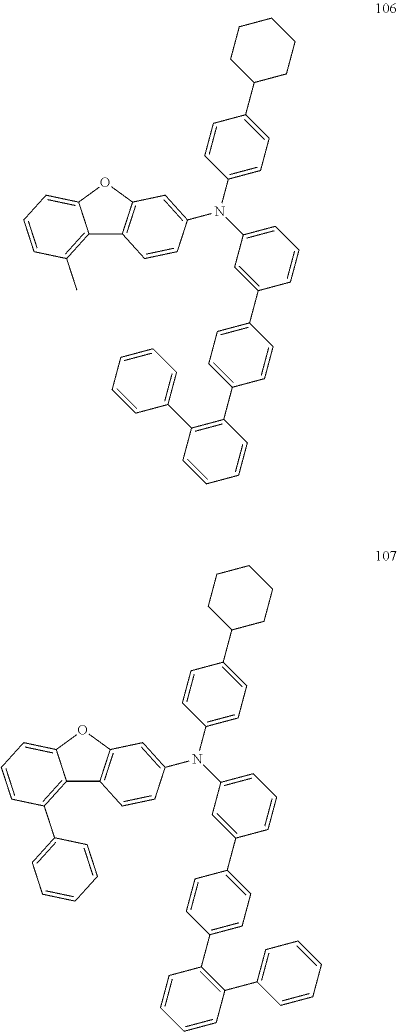 [SMILES: Cc1cccc2oc3cc(N(c4ccc(C5CCCCC5)cc4)c4cccc(-c5ccc(-c6ccccc6-c6ccccc6)cc5)c4)ccc3c12 ; c1ccc(-c2ccccc2-c2ccc(-c3cccc(N(c4ccc(C5CCCCC5)cc4)c4ccc5c(c4)oc4cccc(-c6ccccc6)c45)c3)cc2)cc1] 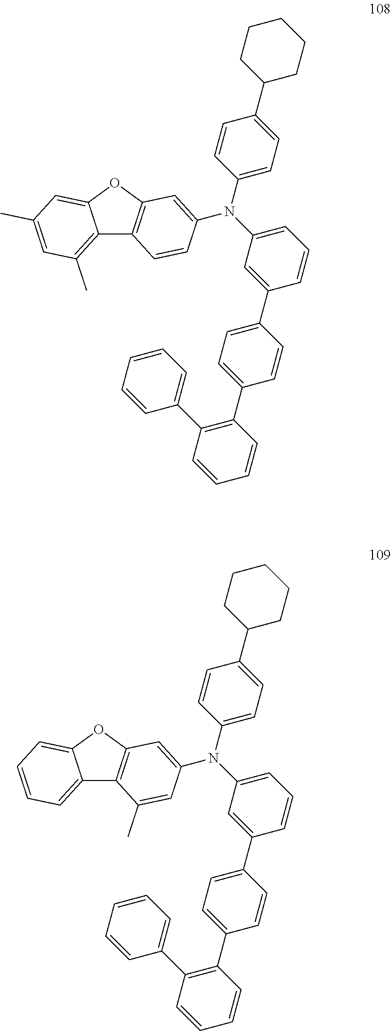 [SMILES: Cc1cc(C)c2c(c1)oc1cc(N(c3ccc(C4CCCCC4)cc3)c3cccc(-c4ccc(-c5ccccc5-c5ccccc5)cc4)c3)ccc12 ; Cc1cc(N(c2ccc(C3CCCCC3)cc2)c2cccc(-c3ccc(-c4ccccc4-c4ccccc4)cc3)c2)cc2oc3ccccc3c12] 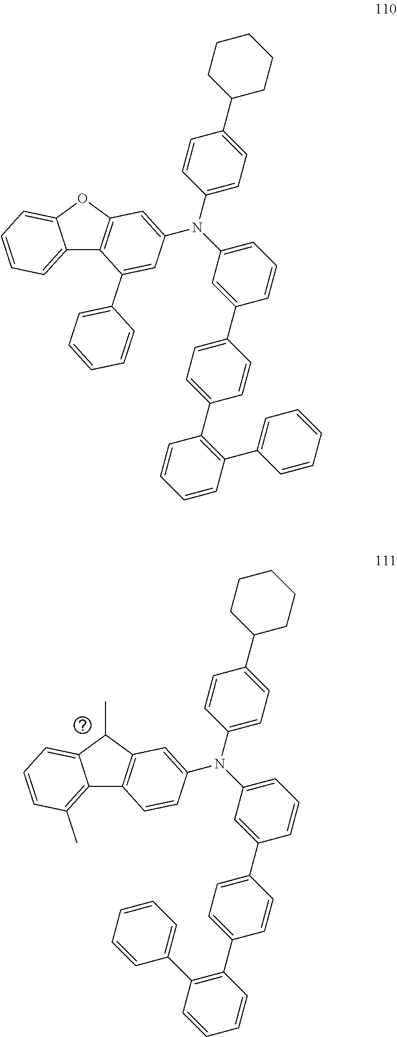 [SMILES: c1ccc(-c2ccccc2-c2ccc(-c3cccc(N(c4ccc(C5CCCCC5)cc4)c4cc(-c5ccccc5)c5c(c4)oc4ccccc45)c3)cc2)cc1 ; Cc1cccc2c1-c1ccc(N(c3ccc(C4CCCCC4)cc3)c3cccc(-c4ccc(-c5ccccc5-c5ccccc5)cc4)c3)cc1C2C] 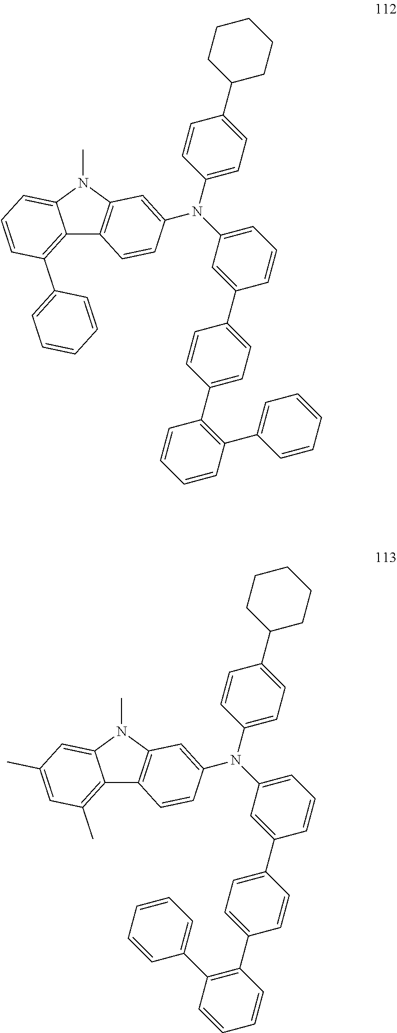 [SMILES: Cn1c2cc(N(c3ccc(C4CCCCC4)cc3)c3cccc(-c4ccc(-c5ccccc5-c5ccccc5)cc4)c3)ccc2c2c(-c3ccccc3)cccc21 ; Cc1cc(C)c2c3ccc(N(c4ccc(C5CCCCC5)cc4)c4cccc(-c5ccc(-c6ccccc6-c6ccccc6)cc5)c4)cc3n(C)c2c1] 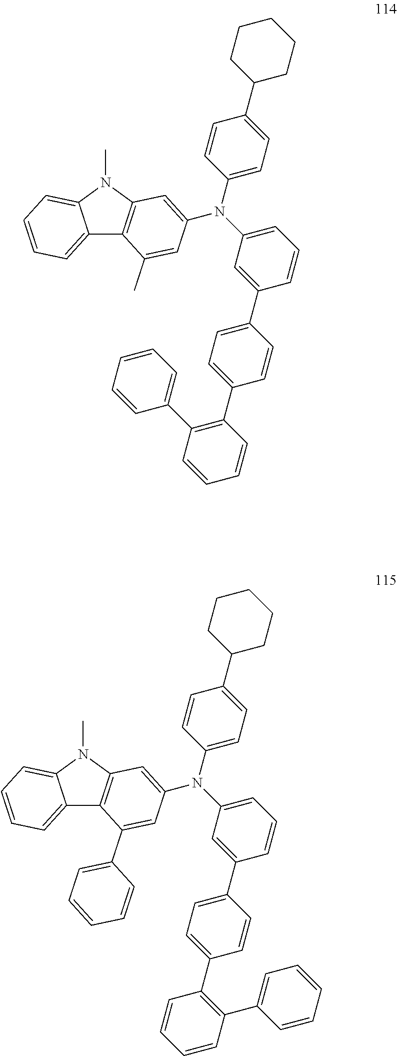 [SMILES: Cc1cc(N(c2ccc(C3CCCCC3)cc2)c2cccc(-c3ccc(-c4ccccc4-c4ccccc4)cc3)c2)cc2c1c1ccccc1n2C ; Cn1c2ccccc2c2c(-c3ccccc3)cc(N(c3ccc(C4CCCCC4)cc3)c3cccc(-c4ccc(-c5ccccc5-c5ccccc5)cc4)c3)cc21] 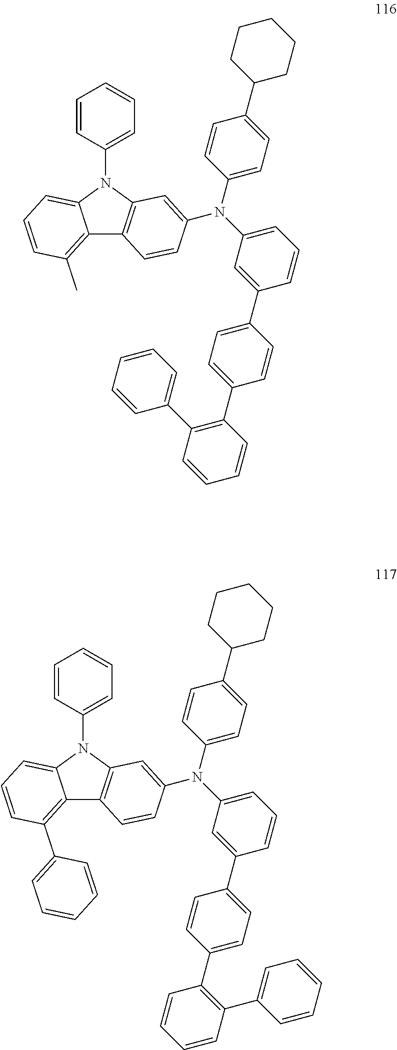 [SMILES: Cc1cccc2c1c1ccc(N(c3ccc(C4CCCCC4)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C5CCCCC5)cc4)c4ccc5c6c(-c7ccccc7)cccc6n(-c6ccccc6)c5c4)c3)cc2)cc1] 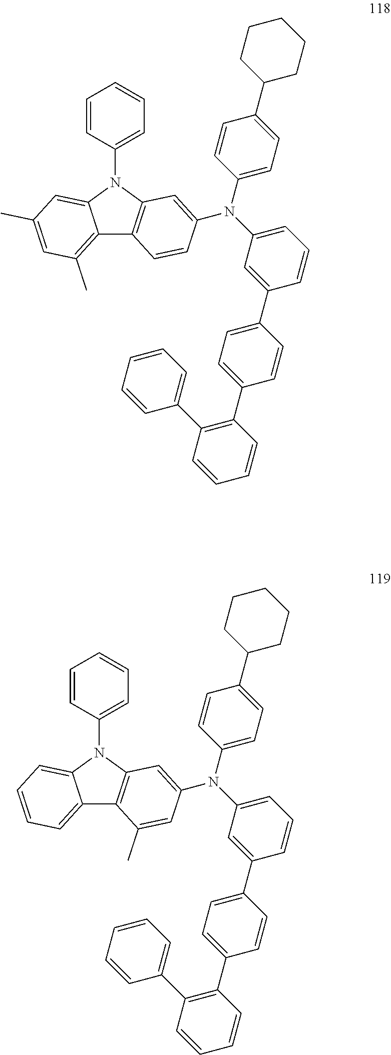 [SMILES: Cc1cc(C)c2c3ccc(N(c4ccc(C5CCCCC5)cc4)c4cccc(-c5ccc(-c6ccccc6-c6ccccc6)cc5)c4)cc3n(-c3ccccc3)c2c1 ; Cc1cc(N(c2ccc(C3CCCCC3)cc2)c2cccc(-c3ccc(-c4ccccc4-c4ccccc4)cc3)c2)cc2c1c1ccccc1n2-c1ccccc1] 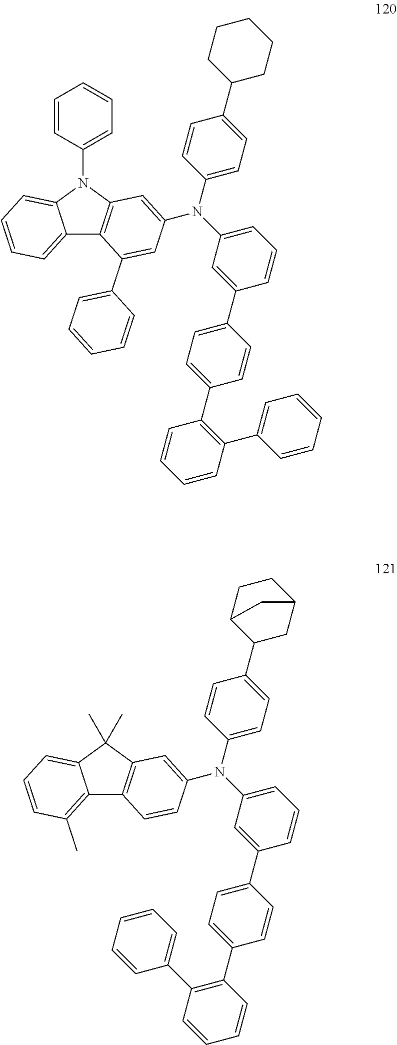 [SMILES: c1ccc(-c2ccccc2-c2ccc(-c3cccc(N(c4ccc(C5CCCCC5)cc4)c4cc(-c5ccccc5)c5c6ccccc6n(-c6ccccc6)c5c4)c3)cc2)cc1 ; Cc1cccc2c1-c1ccc(N(c3ccc(C4CC5CCC4C5)cc3)c3cccc(-c4ccc(-c5ccccc5-c5ccccc5)cc4)c3)cc1C2(C)C] 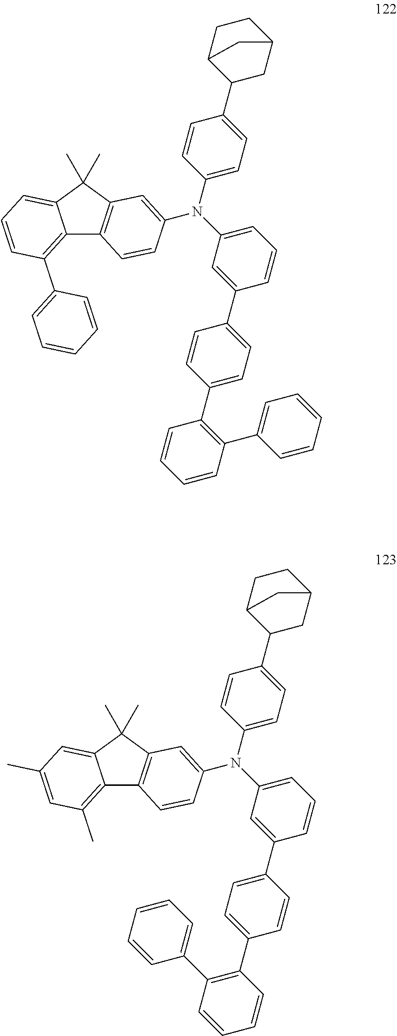 [SMILES: CC1(C)c2cc(N(c3ccc(C4CC5CCC4C5)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C4CC5CCC4C5)cc3)c3cccc(-c4ccc(-c5ccccc5-c5ccccc5)cc4)c3)ccc1-2] 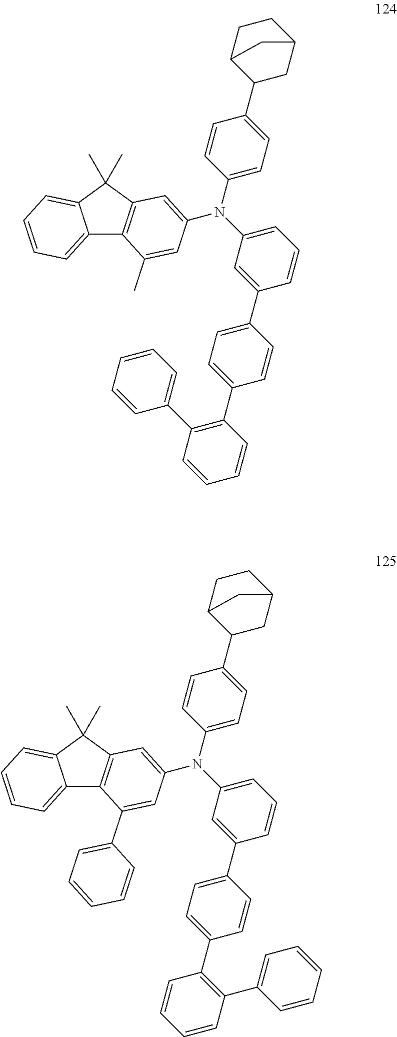 [SMILES: Cc1cc(N(c2ccc(C3CC4CCC3C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C4CC5CCC4C5)cc3)c3cccc(-c4ccc(-c5ccccc5-c5ccccc5)cc4)c3)cc21] 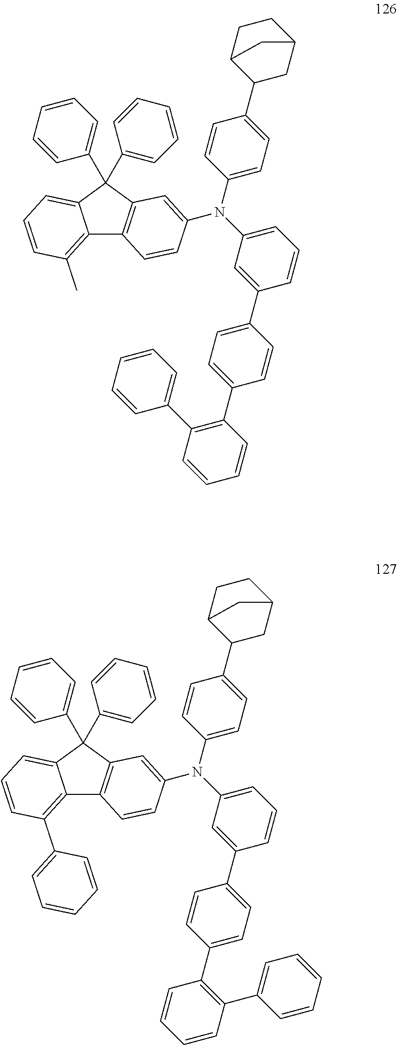 [SMILES: Cc1cccc2c1-c1ccc(N(c3ccc(C4CC5CCC4C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C5CC6CCC5C6)cc4)c4ccc5c(c4)C(c4ccccc4)(c4ccccc4)c4cccc(-c6ccccc6)c4-5)c3)cc2)cc1] 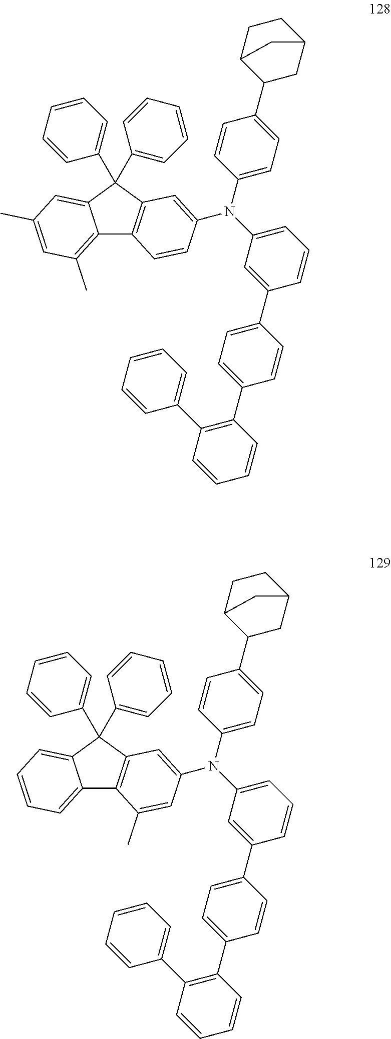 [SMILES: Cc1cc(C)c2c(c1)C(c1ccccc1)(c1ccccc1)c1cc(N(c3ccc(C4CC5CCC4C5)cc3)c3cccc(-c4ccc(-c5ccccc5-c5ccccc5)cc4)c3)ccc1-2 ; Cc1cc(N(c2ccc(C3CC4CCC3C4)cc2)c2cccc(-c3ccc(-c4ccccc4-c4ccccc4)cc3)c2)cc2c1-c1ccccc1C2(c1ccccc1)c1ccccc1] 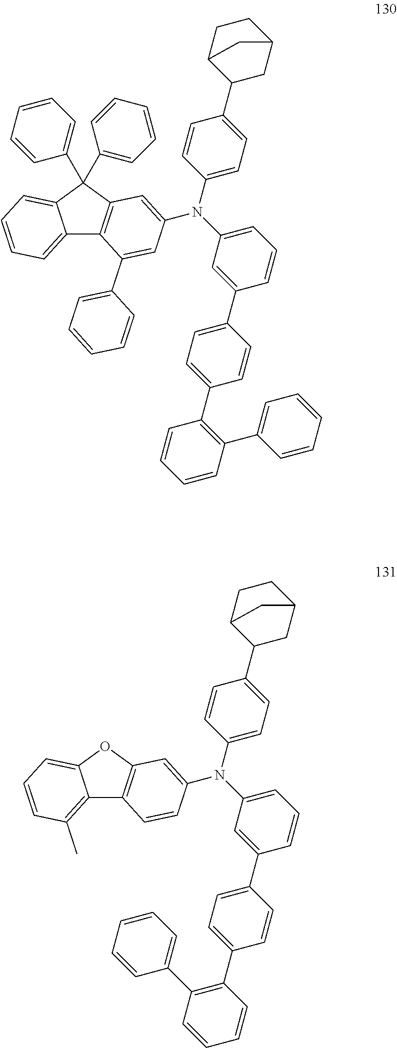 [SMILES: c1ccc(-c2ccccc2-c2ccc(-c3cccc(N(c4ccc(C5CC6CCC5C6)cc4)c4cc(-c5ccccc5)c5c(c4)C(c4ccccc4)(c4ccccc4)c4ccccc4-5)c3)cc2)cc1 ; Cc1cccc2oc3cc(N(c4ccc(C5CC6CCC5C6)cc4)c4cccc(-c5ccc(-c6ccccc6-c6ccccc6)cc5)c4)ccc3c12] 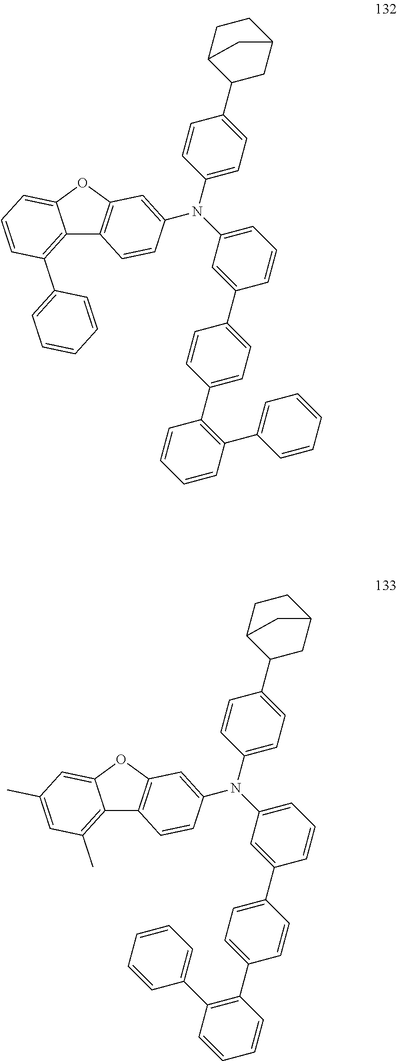 [SMILES: c1ccc(-c2ccccc2-c2ccc(-c3cccc(N(c4ccc(C5CC6CCC5C6)cc4)c4ccc5c(c4)oc4cccc(-c6ccccc6)c45)c3)cc2)cc1 ; Cc1cc(C)c2c(c1)oc1cc(N(c3ccc(C4CC5CCC4C5)cc3)c3cccc(-c4ccc(-c5ccccc5-c5ccccc5)cc4)c3)ccc12] 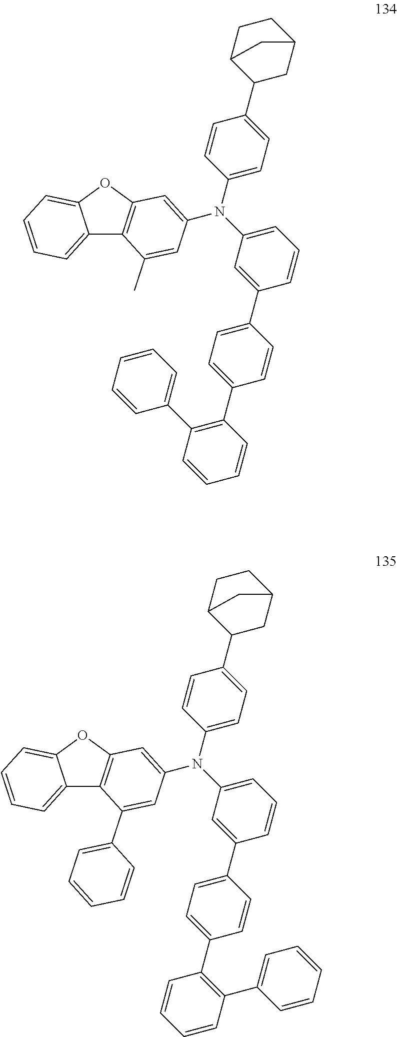 [SMILES: Cc1cc(N(c2ccc(C3CC4CCC3C4)cc2)c2cccc(-c3ccc(-c4ccccc4-c4ccccc4)cc3)c2)cc2oc3ccccc3c12 ; c1ccc(-c2ccccc2-c2ccc(-c3cccc(N(c4ccc(C5CC6CCC5C6)cc4)c4cc(-c5ccccc5)c5c(c4)oc4ccccc45)c3)cc2)cc1] 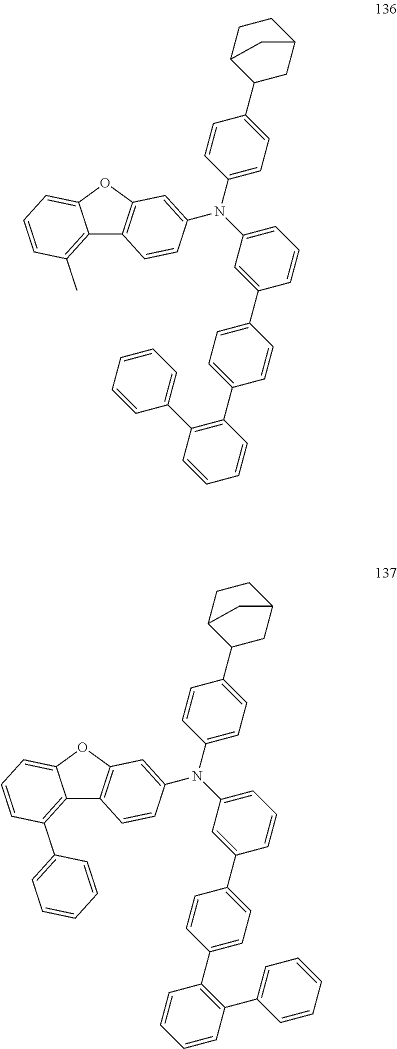 [SMILES: Cc1cccc2oc3cc(N(c4ccc(C5CC6CCC5C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C5CC6CCC5C6)cc4)c4ccc5c(c4)oc4cccc(-c6ccccc6)c45)c3)cc2)cc1] 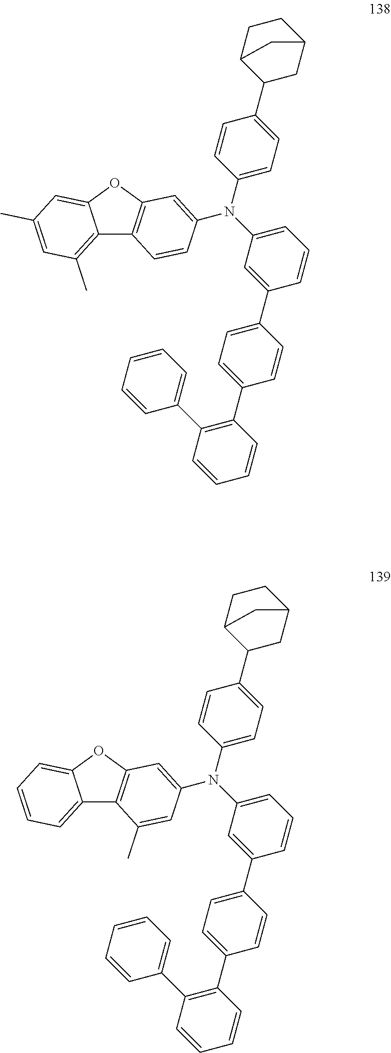 [SMILES: Cc1cc(C)c2c(c1)oc1cc(N(c3ccc(C4CC5CCC4C5)cc3)c3cccc(-c4ccc(-c5ccccc5-c5ccccc5)cc4)c3)ccc12 ; Cc1cc(N(c2ccc(C3CC4CCC3C4)cc2)c2cccc(-c3ccc(-c4ccccc4-c4ccccc4)cc3)c2)cc2oc3ccccc3c12] 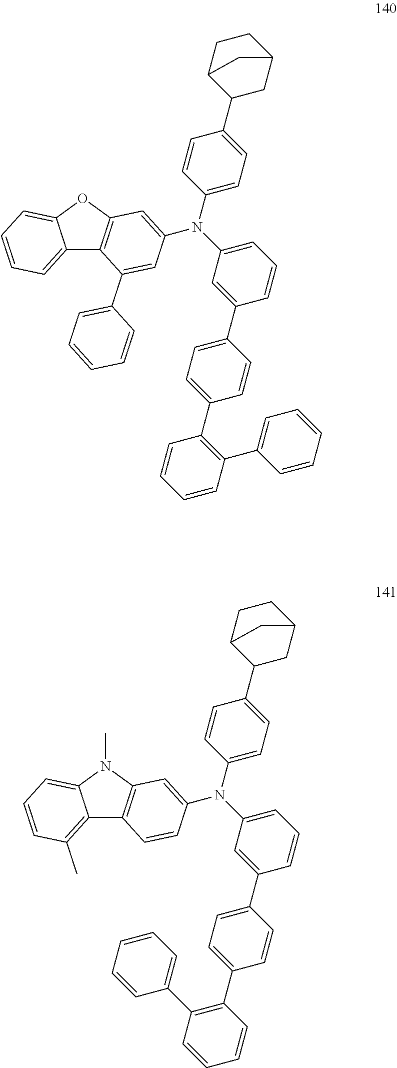 [SMILES: c1ccc(-c2ccccc2-c2ccc(-c3cccc(N(c4ccc(C5CC6CCC5C6)cc4)c4cc(-c5ccccc5)c5c(c4)oc4ccccc45)c3)cc2)cc1 ; Cc1cccc2c1c1ccc(N(c3ccc(C4CC5CCC4C5)cc3)c3cccc(-c4ccc(-c5ccccc5-c5ccccc5)cc4)c3)cc1n2C] 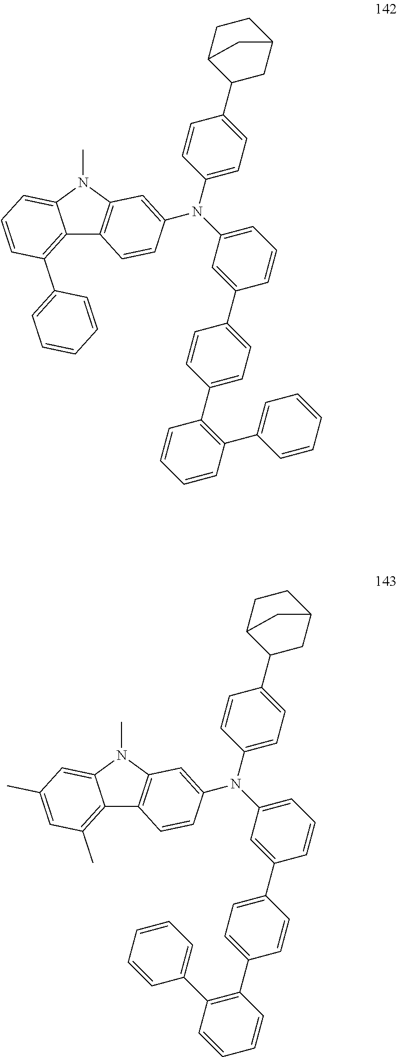 [SMILES: Cn1c2cc(N(c3ccc(C4CC5CCC4C5)cc3)c3cccc(-c4ccc(-c5ccccc5-c5ccccc5)cc4)c3)ccc2c2c(-c3ccccc3)cccc21 ; Cc1cc(C)c2c3ccc(N(c4ccc(C5CC6CCC5C6)cc4)c4cccc(-c5ccc(-c6ccccc6-c6ccccc6)cc5)c4)cc3n(C)c2c1] 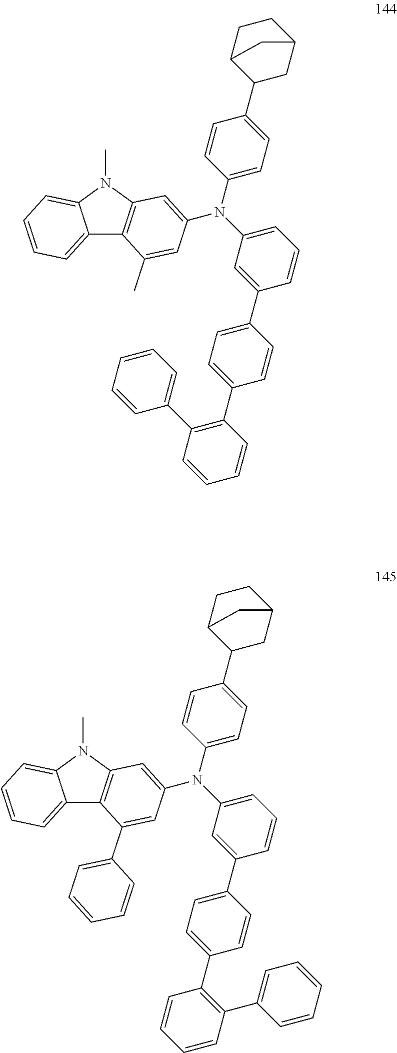 [SMILES: Cc1cc(N(c2ccc(C3CC4CCC3C4)cc2)c2cccc(-c3ccc(-c4ccccc4-c4ccccc4)cc3)c2)cc2c1c1ccccc1n2C ; Cn1c2ccccc2c2c(-c3ccccc3)cc(N(c3ccc(C4CC5CCC4C5)cc3)c3cccc(-c4ccc(-c5ccccc5-c5ccccc5)cc4)c3)cc21] 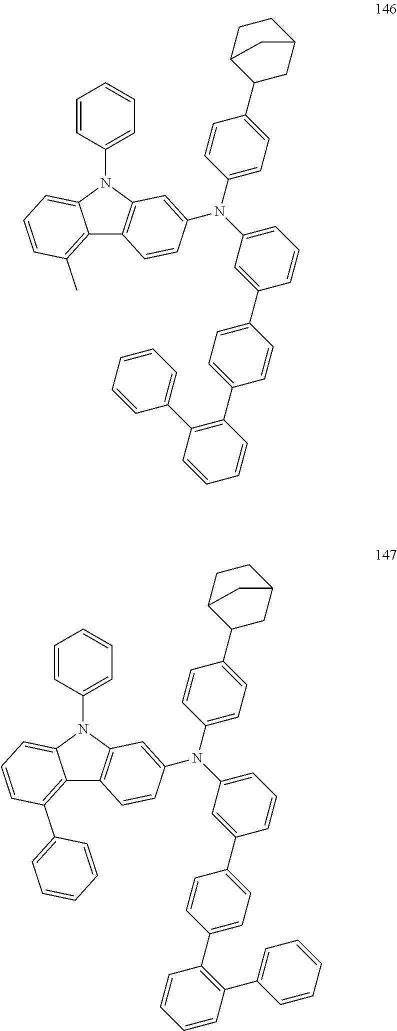 [SMILES: Cc1cccc2c1c1ccc(N(c3ccc(C4CC5CCC4C5)cc3)c3cccc(-c4ccc(-c5ccccc5-c5ccccc5)cc4)c3)cc1n2-c1ccccc1 ; c1ccc(-c2ccccc2-c2ccc(-c3cccc(N(c4ccc(C5CC6CCC5C6)cc4)c4ccc5c6c(-c7ccccc7)cccc6n(-c6ccccc6)c5c4)c3)cc2)cc1] 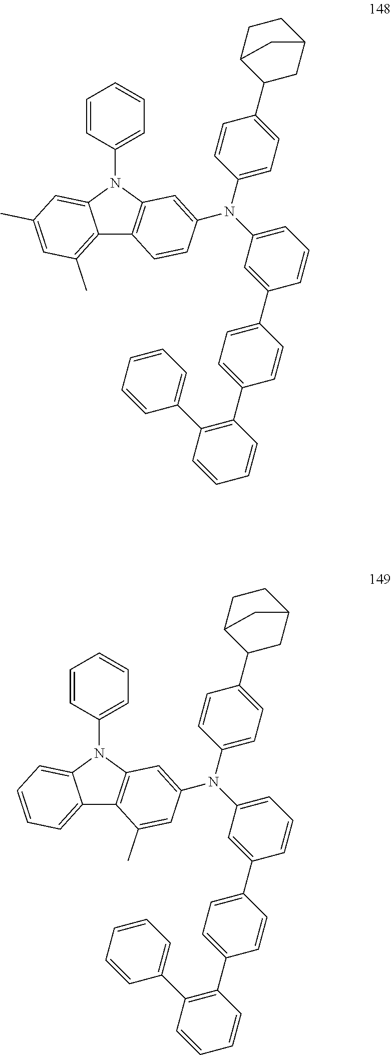 [SMILES: Cc1cc(C)c2c3ccc(N(c4ccc(C5CC6CCC5C6)cc4)c4cccc(-c5ccc(-c6ccccc6-c6ccccc6)cc5)c4)cc3n(-c3ccccc3)c2c1 ; Cc1cc(N(c2ccc(C3CC4CCC3C4)cc2)c2cccc(-c3ccc(-c4ccccc4-c4ccccc4)cc3)c2)cc2c1c1ccccc1n2-c1ccccc1] 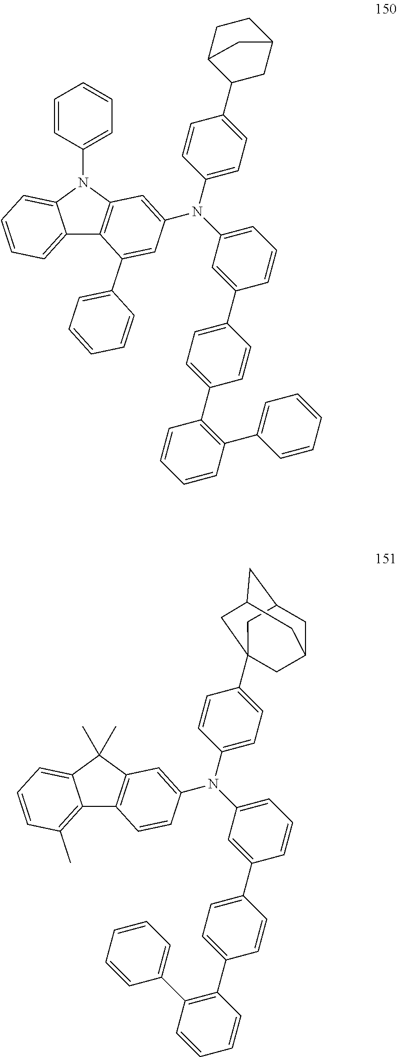 [SMILES: c1ccc(-c2ccccc2-c2ccc(-c3cccc(N(c4ccc(C5CC6CCC5C6)cc4)c4cc(-c5ccccc5)c5c6ccccc6n(-c6ccccc6)c5c4)c3)cc2)cc1 ; Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(C)C] 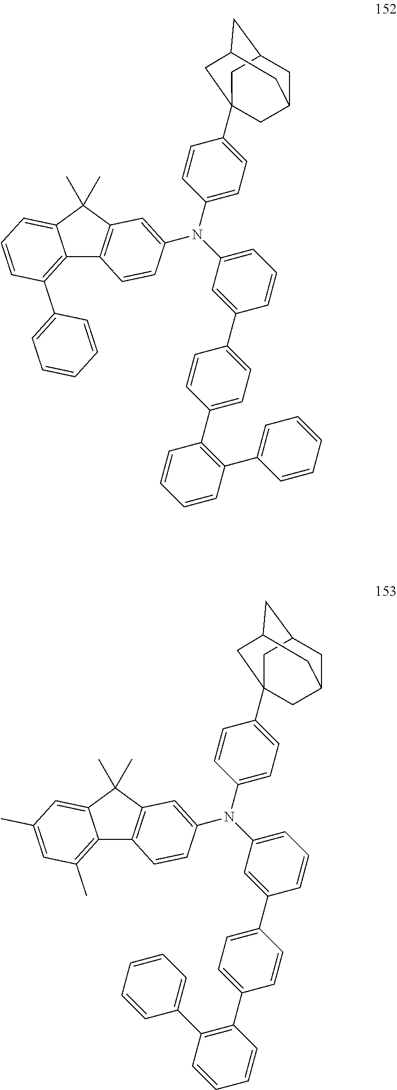 [SMILES: CC1(C)c2cc(N(c3ccc(C45CC6CC(CC(C6)C4)C5)cc3)c3cccc(-c4ccc(-c5ccccc5-c5ccccc5)cc4)c3)ccc2-c2c(-c3ccccc3)cccc21 ; Cc1cc(C)c2c(c1)C(C)(C)c1cc(N(c3ccc(C45CC6CC(CC(C6)C4)C5)cc3)c3cccc(-c4ccc(-c5ccccc5-c5ccccc5)cc4)c3)ccc1-2] 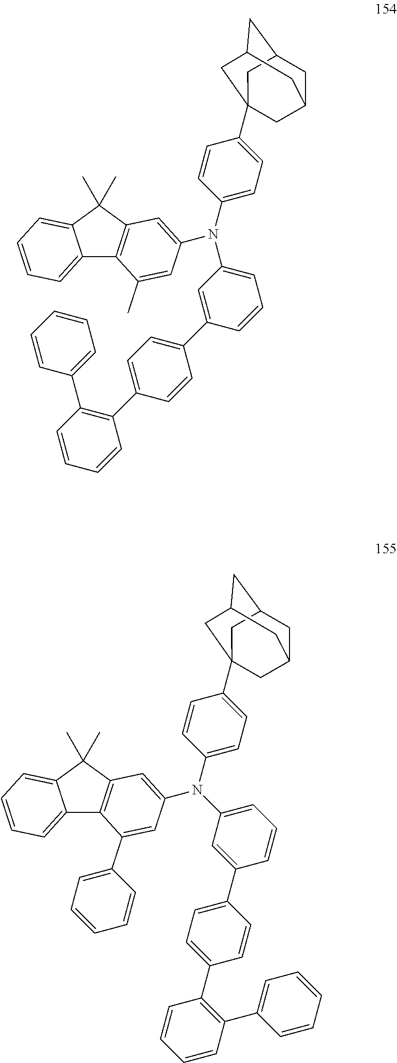 [SMILES: Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(C)C ; CC1(C)c2ccccc2-c2c(-c3ccccc3)cc(N(c3ccc(C45CC6CC(CC(C6)C4)C5)cc3)c3cccc(-c4ccc(-c5ccccc5-c5ccccc5)cc4)c3)cc21] 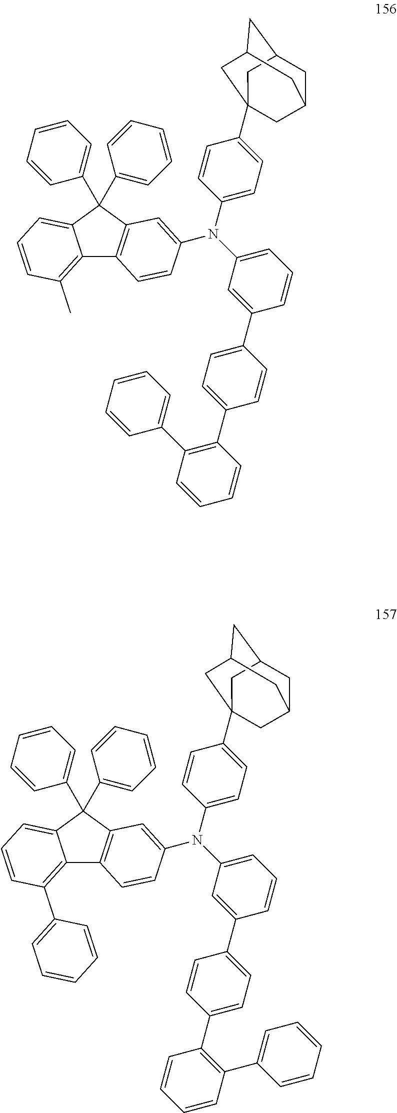 [SMILES: Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2(c1ccccc1)c1ccccc1 ; c1ccc(-c2ccccc2-c2ccc(-c3cccc(N(c4ccc(C56CC7CC(CC(C7)C5)C6)cc4)c4ccc5c(c4)C(c4ccccc4)(c4ccccc4)c4cccc(-c6ccccc6)c4-5)c3)cc2)cc1] 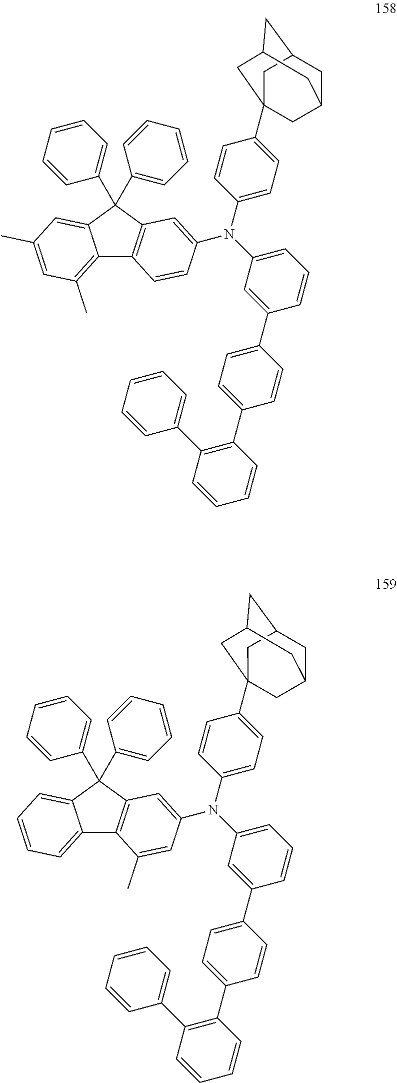 [SMILES: Cc1cc(C)c2c(c1)C(c1ccccc1)(c1ccccc1)c1cc(N(c3ccc(C45CC6CC(CC(C6)C4)C5)cc3)c3cccc(-c4ccc(-c5ccccc5-c5ccccc5)cc4)c3)ccc1-2 ; Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2(c1ccccc1)c1ccccc1] 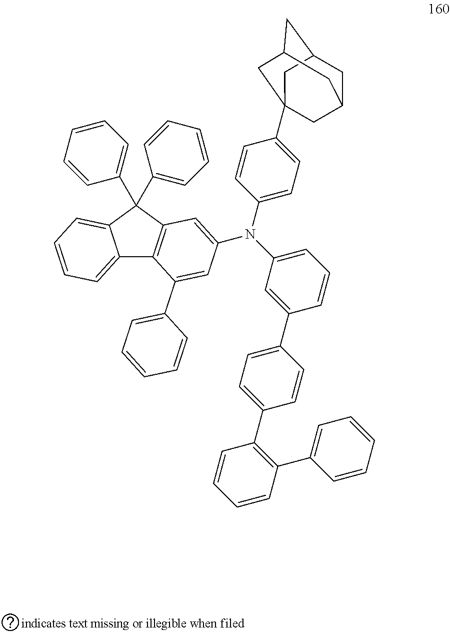 [SMILES: c1ccc(-c2ccccc2-c2ccc(-c3cccc(N(c4ccc(C56CC7CC(CC(C7)C5)C6)cc4)c4cc(-c5ccccc5)c5c(c4)C(c4ccccc4)(c4ccccc4)c4ccccc4-5)c3)cc2)cc1] 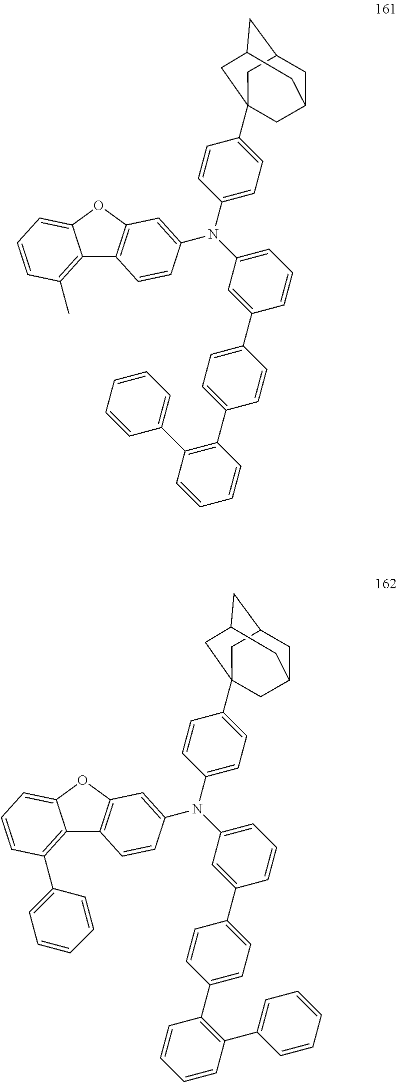 [SMILES: Cc1cccc2oc3cc(N(c4ccc(C56CC7CC(CC(C7)C5)C6)cc4)c4cccc(-c5ccc(-c6ccccc6-c6ccccc6)cc5)c4)ccc3c12 ; c1ccc(-c2ccccc2-c2ccc(-c3cccc(N(c4ccc(C56CC7CC(CC(C7)C5)C6)cc4)c4ccc5c(c4)oc4cccc(-c6ccccc6)c45)c3)cc2)cc1] 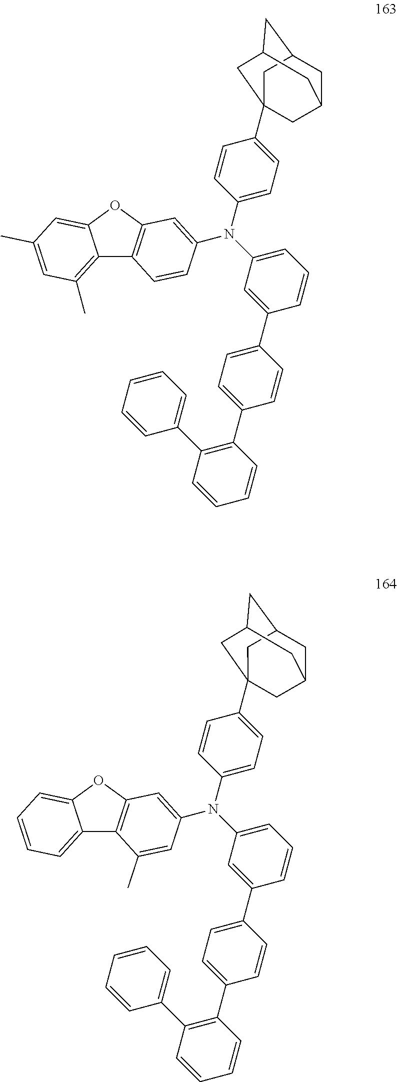 [SMILES: Cc1cc(C)c2c(c1)oc1cc(N(c3ccc(C45CC6CC(CC(C6)C4)C5)cc3)c3cccc(-c4ccc(-c5ccccc5-c5ccccc5)cc4)c3)ccc12 ; Cc1cc(N(c2ccc(C34CC5CC(CC(C5)C3)C4)cc2)c2cccc(-c3ccc(-c4ccccc4-c4ccccc4)cc3)c2)cc2oc3ccccc3c12] 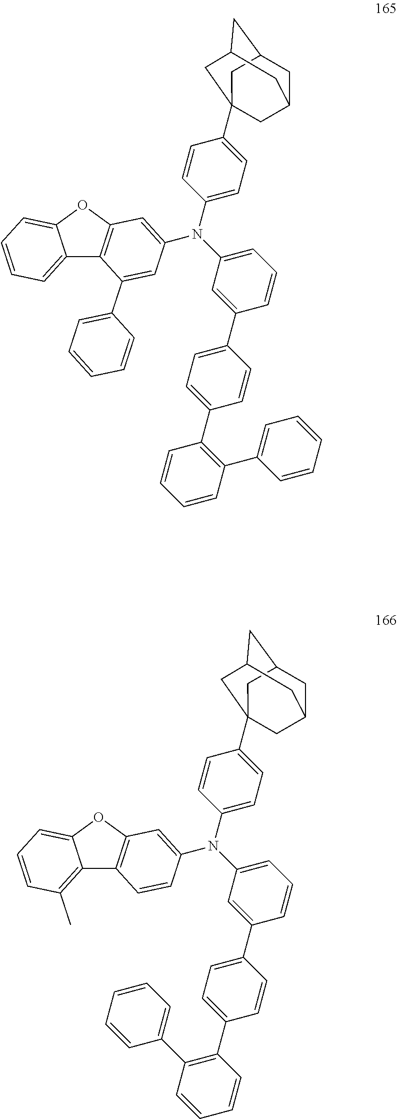 [SMILES: c1ccc(-c2ccccc2-c2ccc(-c3cccc(N(c4ccc(C56CC7CC(CC(C7)C5)C6)cc4)c4cc(-c5ccccc5)c5c(c4)oc4ccccc45)c3)cc2)cc1 ; Cc1cccc2oc3cc(N(c4ccc(C56CC7CC(CC(C7)C5)C6)cc4)c4cccc(-c5ccc(-c6ccccc6-c6ccccc6)cc5)c4)ccc3c12] 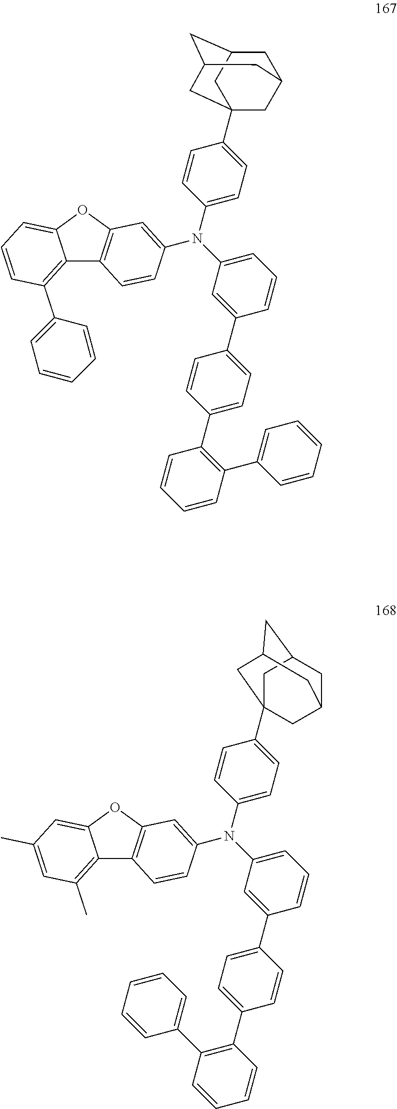 [SMILES: c1ccc(-c2ccccc2-c2ccc(-c3cccc(N(c4ccc(C56CC7CC(CC(C7)C5)C6)cc4)c4ccc5c(c4)oc4cccc(-c6ccccc6)c45)c3)cc2)cc1 ; Cc1cc(C)c2c(c1)oc1cc(N(c3ccc(C45CC6CC(CC(C6)C4)C5)cc3)c3cccc(-c4ccc(-c5ccccc5-c5ccccc5)cc4)c3)ccc12] 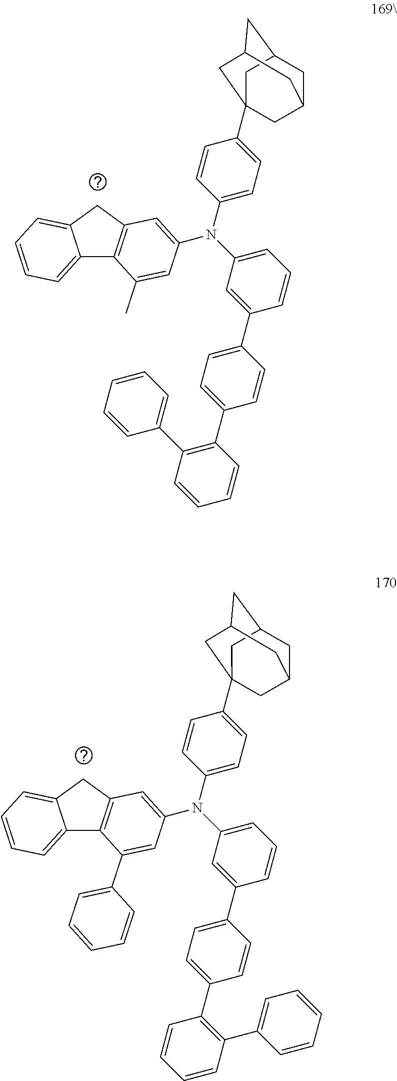 [SMILES: Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2 ; c1ccc(-c2ccccc2-c2ccc(-c3cccc(N(c4ccc(C56CC7CC(CC(C7)C5)C6)cc4)c4cc5c(c(-c6ccccc6)c4)-c4ccccc4C5)c3)cc2)cc1] 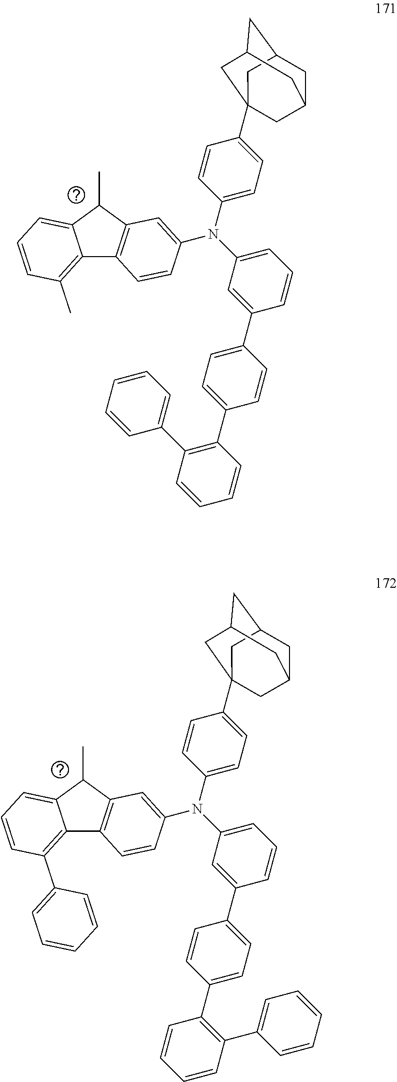 [SMILES: Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2C ; CC1c2cc(N(c3ccc(C45CC6CC(CC(C6)C4)C5)cc3)c3cccc(-c4ccc(-c5ccccc5-c5ccccc5)cc4)c3)ccc2-c2c(-c3ccccc3)cccc21] 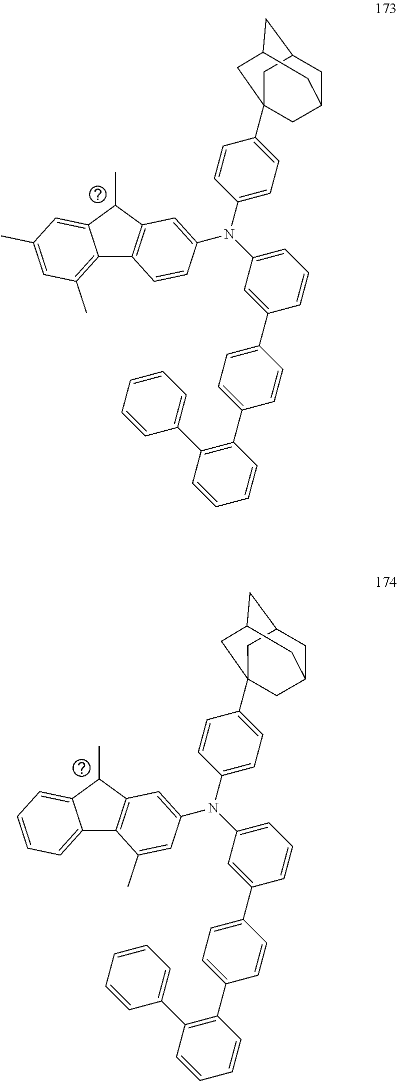 [SMILES: Cc1cc(C)c2c(c1)C(C)c1cc(N(c3ccc(C45CC6CC(CC(C6)C4)C5)cc3)c3cccc(-c4ccc(-c5ccccc5-c5ccccc5)cc4)c3)ccc1-2 ; Cc1cc(N(c2ccc(C34CC5CC(CC(C5)C3)C4)cc2)c2cccc(-c3ccc(-c4ccccc4-c4ccccc4)cc3)c2)cc2c1-c1ccccc1C2C] 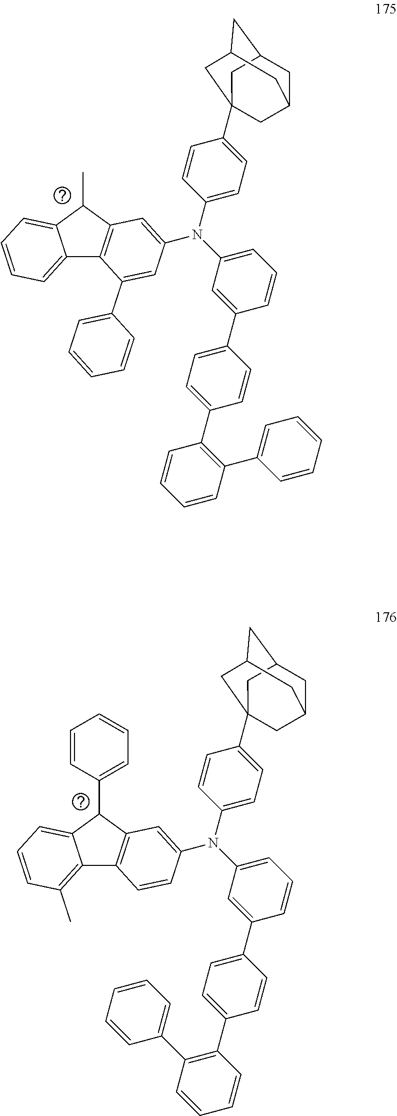 [SMILES: CC1c2ccccc2-c2c(-c3ccccc3)cc(N(c3ccc(C45CC6CC(CC(C6)C4)C5)cc3)c3cccc(-c4ccc(-c5ccccc5-c5ccccc5)cc4)c3)cc21 ; Cc1cccc2c1-c1ccc(N(c3ccc(C45CC6CC(CC(C6)C4)C5)cc3)c3cccc(-c4ccc(-c5ccccc5-c5ccccc5)cc4)c3)cc1C2c1ccccc1] 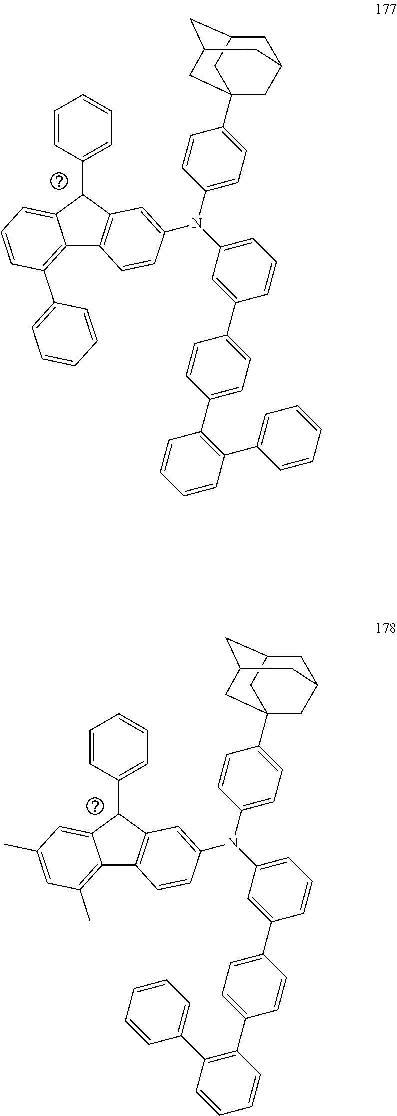 [SMILES: c1ccc(-c2ccccc2-c2ccc(-c3cccc(N(c4ccc(C56CC7CC(CC(C7)C5)C6)cc4)c4ccc5c(c4)C(c4ccccc4)c4cccc(-c6ccccc6)c4-5)c3)cc2)cc1 ; Cc1cc(C)c2c(c1)C(c1ccccc1)c1cc(N(c3ccc(C45CC6CC(CC(C6)C4)C5)cc3)c3cccc(-c4ccc(-c5ccccc5-c5ccccc5)cc4)c3)ccc1-2] 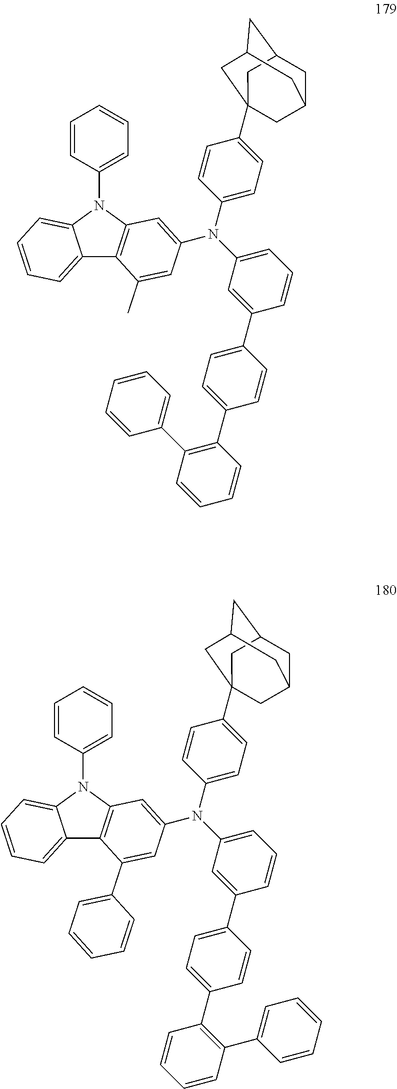 [SMILES: Cc1cc(N(c2ccc(C34CC5CC(CC(C5)C3)C4)cc2)c2cccc(-c3ccc(-c4ccccc4-c4ccccc4)cc3)c2)cc2c1c1ccccc1n2-c1ccccc1 ; c1ccc(-c2ccccc2-c2ccc(-c3cccc(N(c4ccc(C56CC7CC(CC(C7)C5)C6)cc4)c4cc(-c5ccccc5)c5c6ccccc6n(-c6ccccc6)c5c4)c3)cc2)cc1] 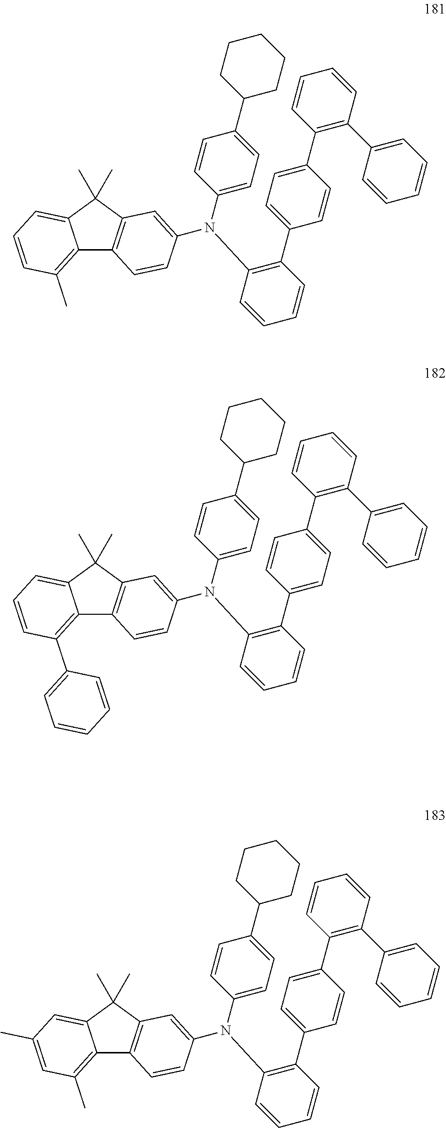 [SMILES: Cc1cccc2c1-c1ccc(N(c3ccc(C4CCCCC4)cc3)c3ccccc3-c3ccc(-c4ccccc4-c4ccccc4)cc3)cc1C2(C)C ; CC1(C)c2cc(N(c3ccc(C4CCCCC4)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CCCCC4)cc3)c3ccccc3-c3ccc(-c4ccccc4-c4ccccc4)cc3)ccc1-2] 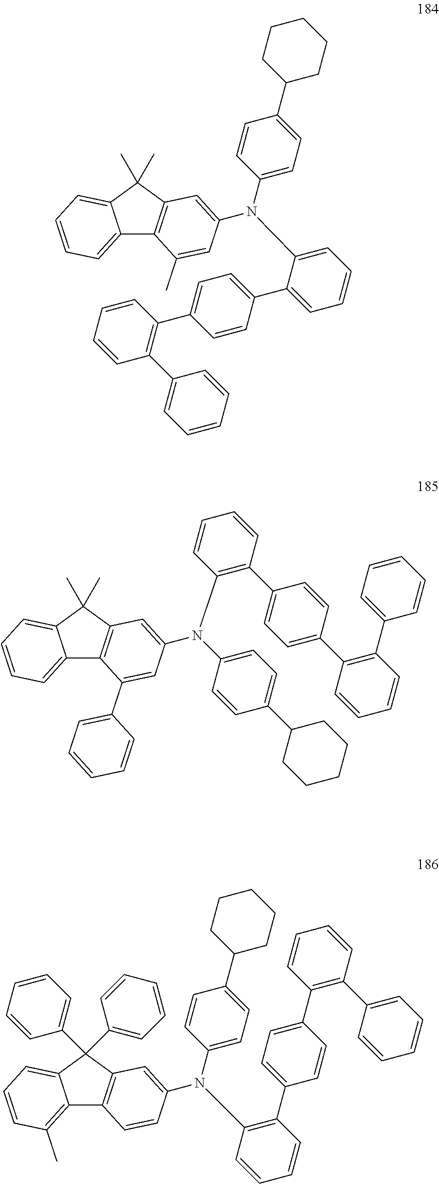 [SMILES: Cc1cc(N(c2ccc(C3CCCCC3)cc2)c2ccccc2-c2ccc(-c3ccccc3-c3ccccc3)cc2)cc2c1-c1ccccc1C2(C)C ; CC1(C)c2ccccc2-c2c(-c3ccccc3)cc(N(c3ccc(C4CCCCC4)cc3)c3ccccc3-c3ccc(-c4ccccc4-c4ccccc4)cc3)cc21 ; Cc1cccc2c1-c1ccc(N(c3ccc(C4CCCCC4)cc3)c3ccccc3-c3ccc(-c4ccccc4-c4ccccc4)cc3)cc1C2(c1ccccc1)c1ccccc1] 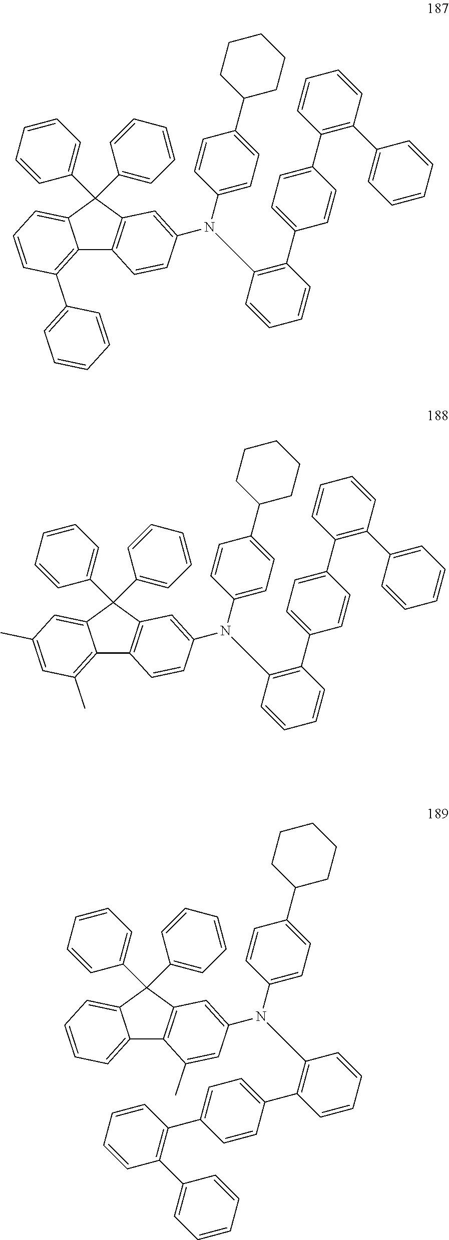 [SMILES: c1ccc(-c2ccccc2-c2ccc(-c3ccccc3N(c3ccc(C4CCCCC4)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C4CCCCC4)cc3)c3ccccc3-c3ccc(-c4ccccc4-c4ccccc4)cc3)ccc1-2 ; Cc1cc(N(c2ccc(C3CCCCC3)cc2)c2ccccc2-c2ccc(-c3ccccc3-c3ccccc3)cc2)cc2c1-c1ccccc1C2(c1ccccc1)c1ccccc1] 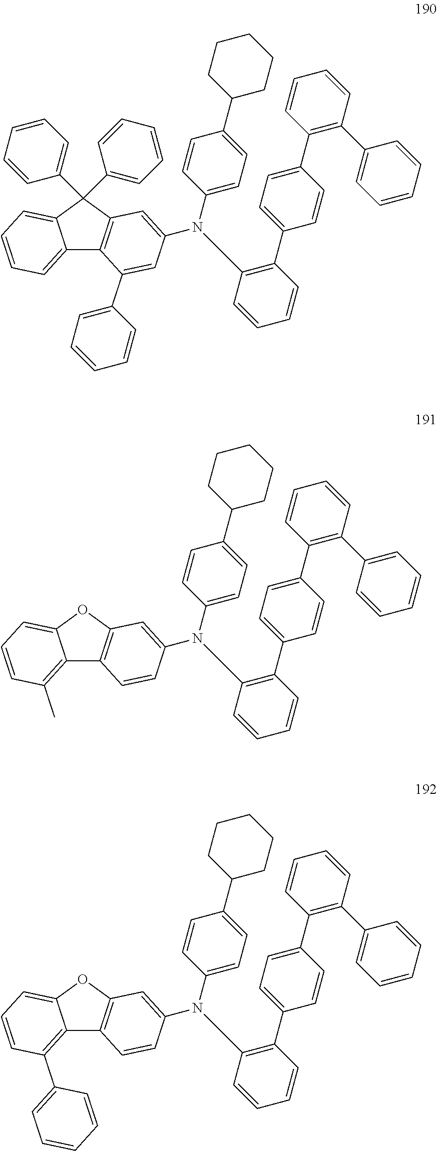 [SMILES: c1ccc(-c2ccccc2-c2ccc(-c3ccccc3N(c3ccc(C4CCCCC4)cc3)c3cc(-c4ccccc4)c4c(c3)C(c3ccccc3)(c3ccccc3)c3ccccc3-4)cc2)cc1 ; Cc1cccc2oc3cc(N(c4ccc(C5CCCCC5)cc4)c4ccccc4-c4ccc(-c5ccccc5-c5ccccc5)cc4)ccc3c12 ; c1ccc(-c2ccccc2-c2ccc(-c3ccccc3N(c3ccc(C4CCCCC4)cc3)c3ccc4c(c3)oc3cccc(-c5ccccc5)c34)cc2)cc1] 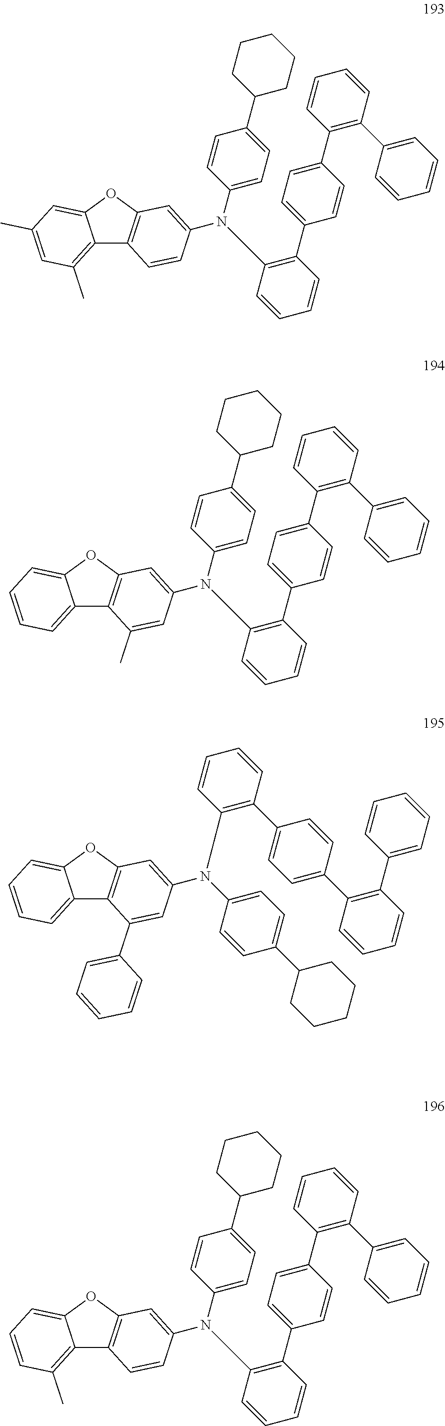 [SMILES: Cc1cc(C)c2c(c1)oc1cc(N(c3ccc(C4CCCCC4)cc3)c3ccccc3-c3ccc(-c4ccccc4-c4ccccc4)cc3)ccc12 ; Cc1cc(N(c2ccc(C3CCCCC3)cc2)c2ccccc2-c2ccc(-c3ccccc3-c3ccccc3)cc2)cc2oc3ccccc3c12 ; c1ccc(-c2ccccc2-c2ccc(-c3ccccc3N(c3ccc(C4CCCCC4)cc3)c3cc(-c4ccccc4)c4c(c3)oc3ccccc34)cc2)cc1 ; Cc1cccc2oc3cc(N(c4ccc(C5CCCCC5)cc4)c4ccccc4-c4ccc(-c5ccccc5-c5ccccc5)cc4)ccc3c12] 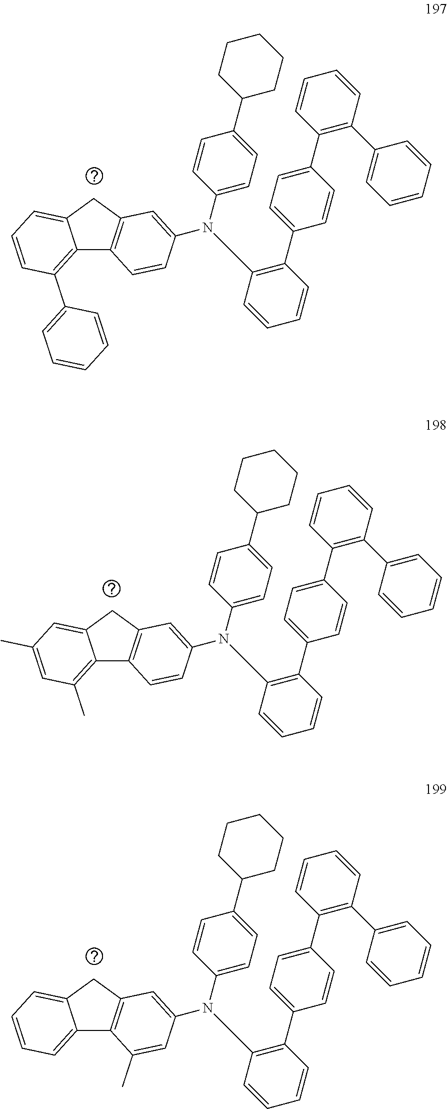 [SMILES: c1ccc(-c2ccccc2-c2ccc(-c3ccccc3N(c3ccc(C4CCCCC4)cc3)c3ccc4c(c3)Cc3cccc(-c5ccccc5)c3-4)cc2)cc1 ; Cc1cc(C)c2c(c1)Cc1cc(N(c3ccc(C4CCCCC4)cc3)c3ccccc3-c3ccc(-c4ccccc4-c4ccccc4)cc3)ccc1-2 ; Cc1cc(N(c2ccc(C3CCCCC3)cc2)c2ccccc2-c2ccc(-c3ccccc3-c3ccccc3)cc2)cc2c1-c1ccccc1C2] 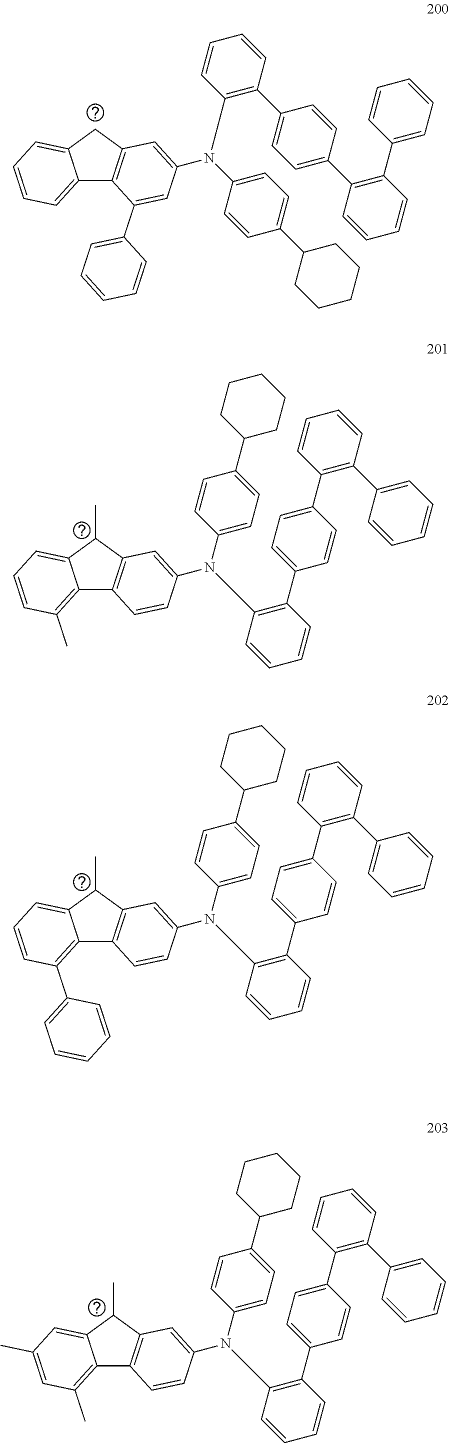 [SMILES: c1ccc(-c2ccccc2-c2ccc(-c3ccccc3N(c3ccc(C4CCCCC4)cc3)c3cc4c(c(-c5ccccc5)c3)-c3ccccc3C4)cc2)cc1 ; Cc1cccc2c1-c1ccc(N(c3ccc(C4CCCCC4)cc3)c3ccccc3-c3ccc(-c4ccccc4-c4ccccc4)cc3)cc1C2C ; CC1c2cc(N(c3ccc(C4CCCCC4)cc3)c3ccccc3-c3ccc(-c4ccccc4-c4ccccc4)cc3)ccc2-c2c(-c3ccccc3)cccc21 ; Cc1cc(C)c2c(c1)C(C)c1cc(N(c3ccc(C4CCCCC4)cc3)c3ccccc3-c3ccc(-c4ccccc4-c4ccccc4)cc3)ccc1-2] 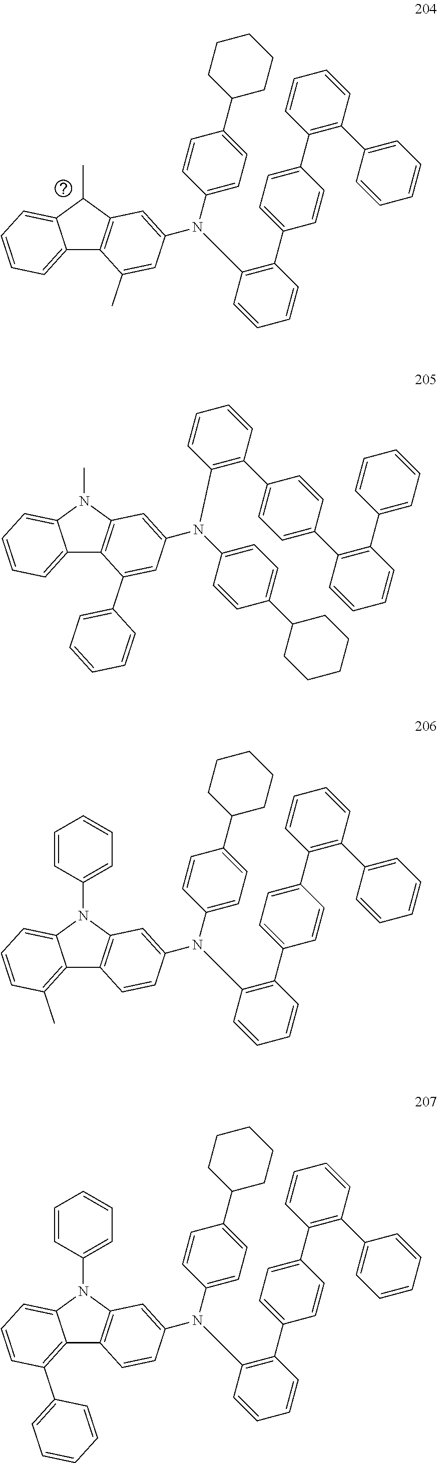 [SMILES: Cc1cc(N(c2ccc(C3CCCCC3)cc2)c2ccccc2-c2ccc(-c3ccccc3-c3ccccc3)cc2)cc2c1-c1ccccc1C2C ; Cn1c2ccccc2c2c(-c3ccccc3)cc(N(c3ccc(C4CCCCC4)cc3)c3ccccc3-c3ccc(-c4ccccc4-c4ccccc4)cc3)cc21 ; Cc1cccc2c1c1ccc(N(c3ccc(C4CCCCC4)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C4CCCCC4)cc3)c3ccc4c5c(-c6ccccc6)cccc5n(-c5ccccc5)c4c3)cc2)cc1] 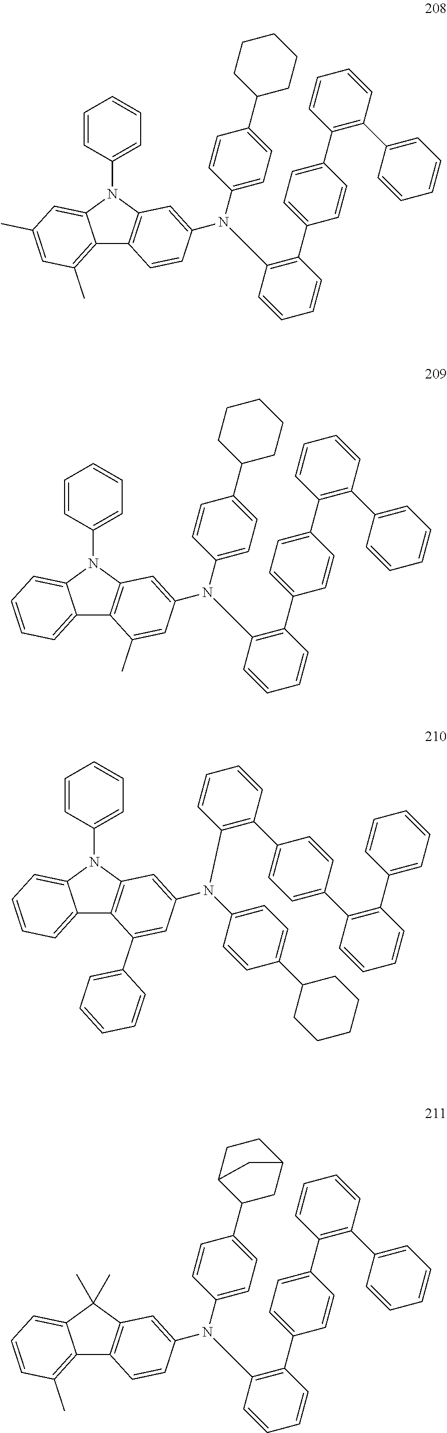 [SMILES: Cc1cc(C)c2c3ccc(N(c4ccc(C5CCCCC5)cc4)c4ccccc4-c4ccc(-c5ccccc5-c5ccccc5)cc4)cc3n(-c3ccccc3)c2c1 ; Cc1cc(N(c2ccc(C3CCCCC3)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CCCCC4)cc3)c3cc(-c4ccccc4)c4c5ccccc5n(-c5ccccc5)c4c3)cc2)cc1 ; Cc1cccc2c1-c1ccc(N(c3ccc(C4CC5CCC4C5)cc3)c3ccccc3-c3ccc(-c4ccccc4-c4ccccc4)cc3)cc1C2(C)C] 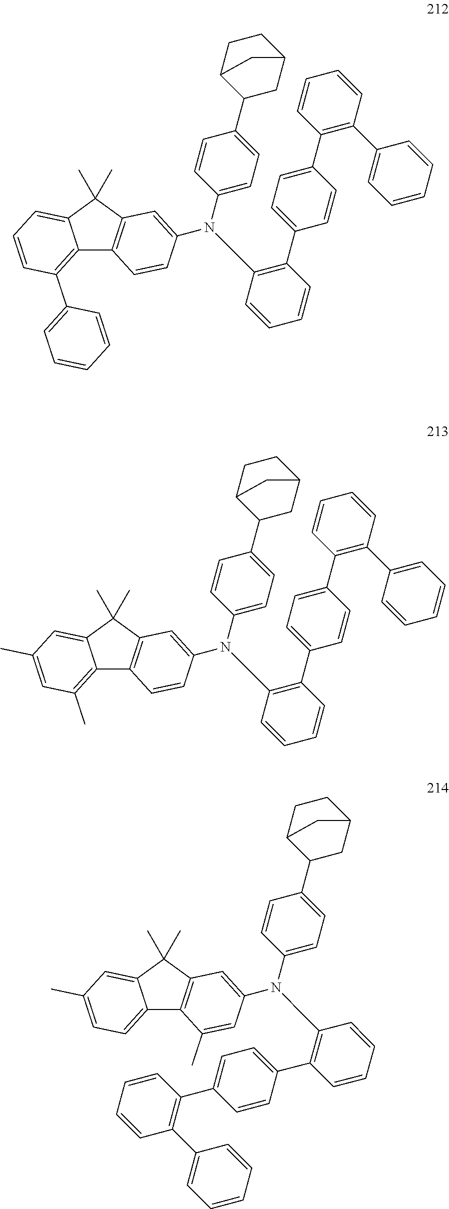 [SMILES: CC1(C)c2cc(N(c3ccc(C4CC5CCC4C5)cc3)c3ccccc3-c3ccc(-c4ccccc4-c4ccccc4)cc3)ccc2-c2c(-c3ccccc3)cccc21 ; Cc1cc(C)c2c(c1)C(C)(C)c1cc(N(c3ccc(C4CC5CCC4C5)cc3)c3ccccc3-c3ccc(-c4ccccc4-c4ccccc4)cc3)ccc1-2 ; Cc1ccc2c(c1)C(C)(C)c1cc(N(c3ccc(C4CC5CCC4C5)cc3)c3ccccc3-c3ccc(-c4ccccc4-c4ccccc4)cc3)cc(C)c1-2] 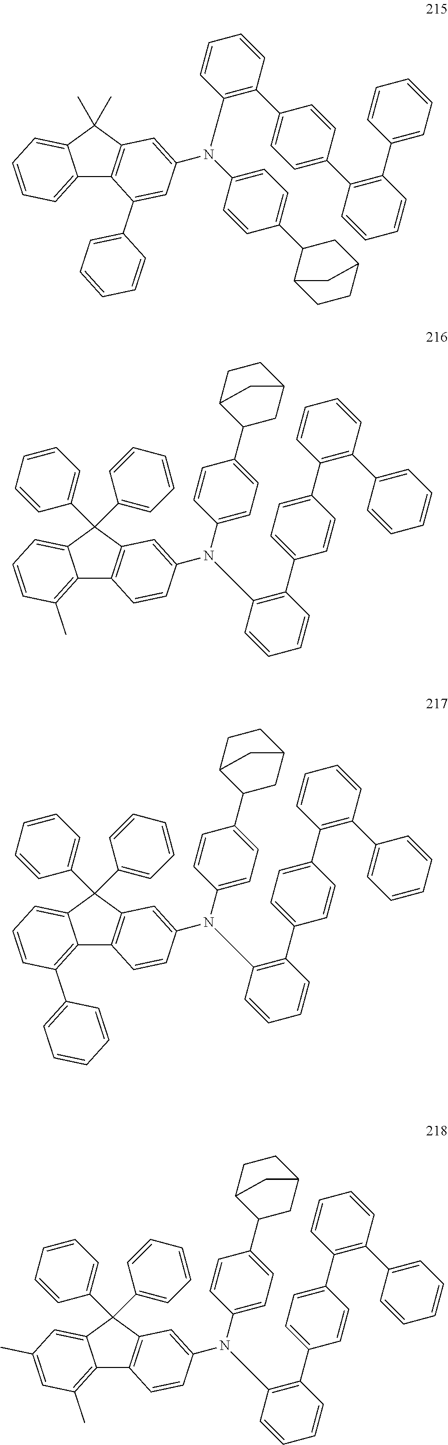 [SMILES: CC1(C)c2ccccc2-c2c(-c3ccccc3)cc(N(c3ccc(C4CC5CCC4C5)cc3)c3ccccc3-c3ccc(-c4ccccc4-c4ccccc4)cc3)cc21 ; Cc1cccc2c1-c1ccc(N(c3ccc(C4CC5CCC4C5)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C4CC5CCC4C5)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C4CC5CCC4C5)cc3)c3ccccc3-c3ccc(-c4ccccc4-c4ccccc4)cc3)ccc1-2] 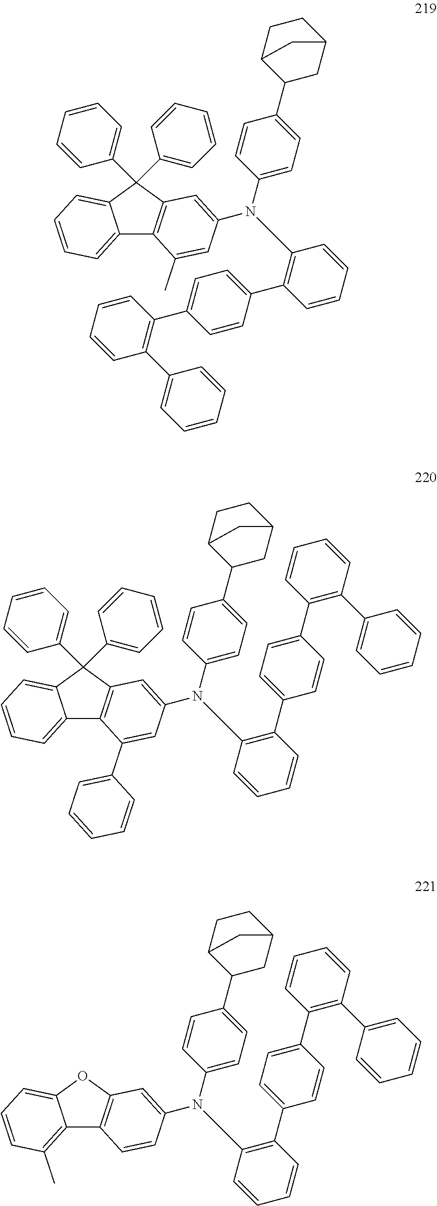 [SMILES: Cc1cc(N(c2ccc(C3CC4CCC3C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C4CC5CCC4C5)cc3)c3cc(-c4ccccc4)c4c(c3)C(c3ccccc3)(c3ccccc3)c3ccccc3-4)cc2)cc1 ; Cc1cccc2oc3cc(N(c4ccc(C5CC6CCC5C6)cc4)c4ccccc4-c4ccc(-c5ccccc5-c5ccccc5)cc4)ccc3c12] 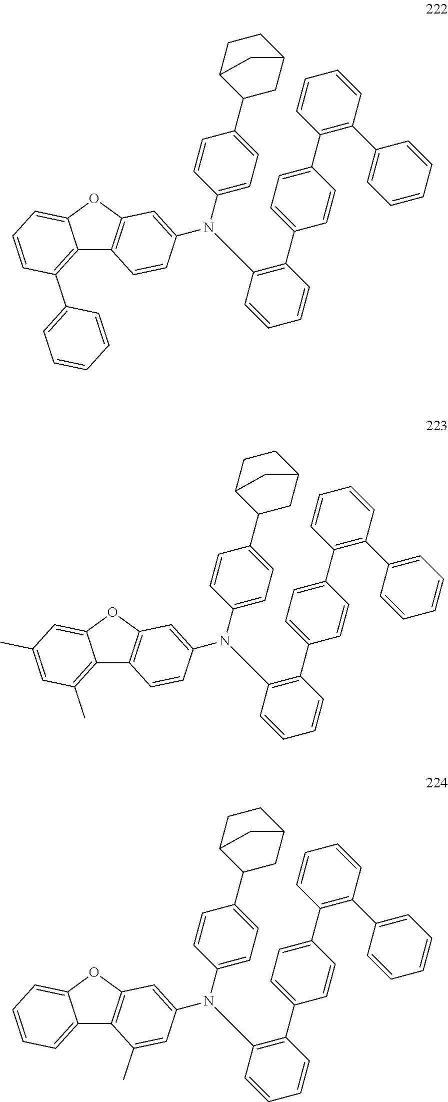 [SMILES: c1ccc(-c2ccccc2-c2ccc(-c3ccccc3N(c3ccc(C4CC5CCC4C5)cc3)c3ccc4c(c3)oc3cccc(-c5ccccc5)c34)cc2)cc1 ; Cc1cc(C)c2c(c1)oc1cc(N(c3ccc(C4CC5CCC4C5)cc3)c3ccccc3-c3ccc(-c4ccccc4-c4ccccc4)cc3)ccc12 ; Cc1cc(N(c2ccc(C3CC4CCC3C4)cc2)c2ccccc2-c2ccc(-c3ccccc3-c3ccccc3)cc2)cc2oc3ccccc3c12] 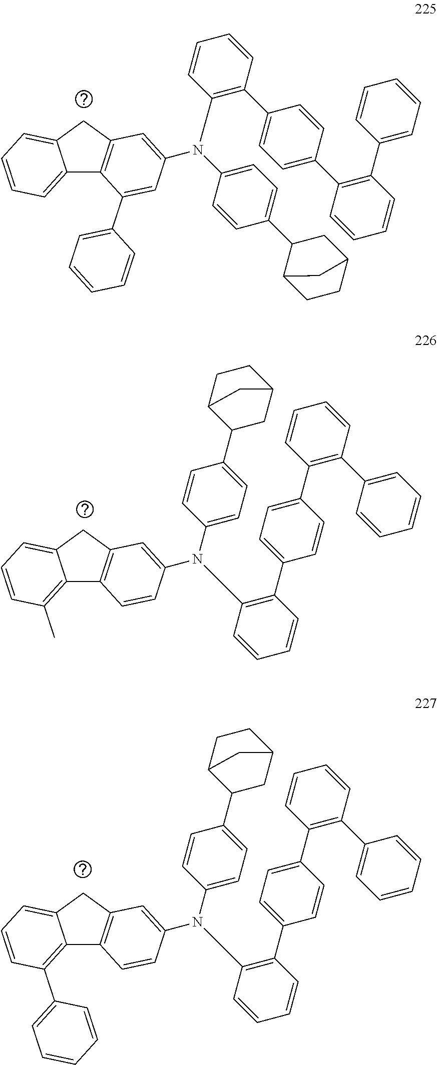 [SMILES: c1ccc(-c2ccccc2-c2ccc(-c3ccccc3N(c3ccc(C4CC5CCC4C5)cc3)c3cc4c(c(-c5ccccc5)c3)-c3ccccc3C4)cc2)cc1 ; Cc1cccc2c1-c1ccc(N(c3ccc(C4CC5CCC4C5)cc3)c3ccccc3-c3ccc(-c4ccccc4-c4ccccc4)cc3)cc1C2 ; c1ccc(-c2ccccc2-c2ccc(-c3ccccc3N(c3ccc(C4CC5CCC4C5)cc3)c3ccc4c(c3)Cc3cccc(-c5ccccc5)c3-4)cc2)cc1] 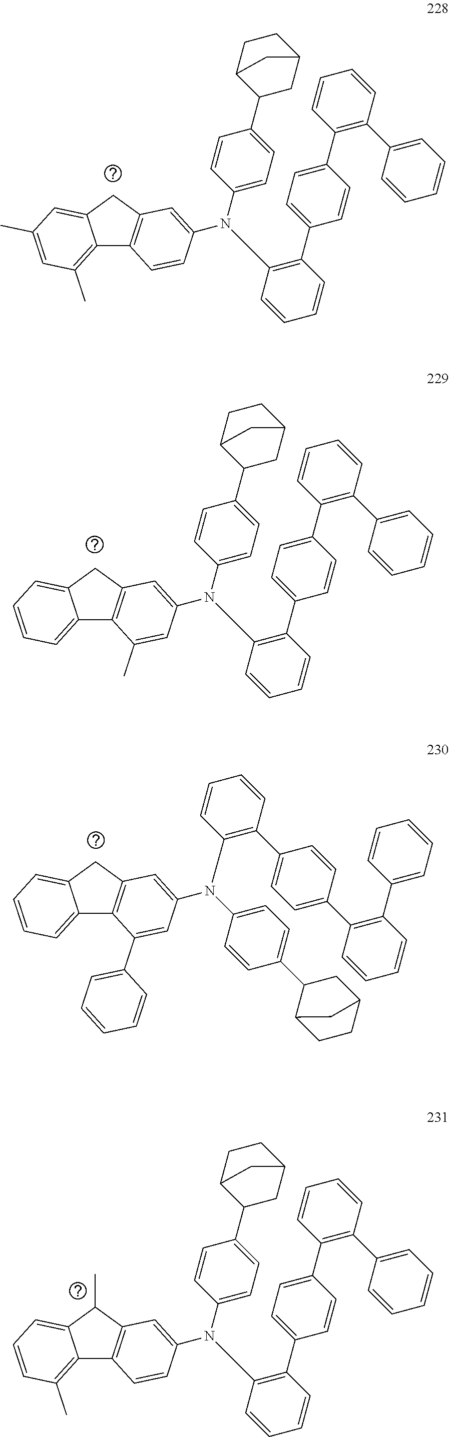 [SMILES: Cc1cc(C)c2c(c1)Cc1cc(N(c3ccc(C4CC5CCC4C5)cc3)c3ccccc3-c3ccc(-c4ccccc4-c4ccccc4)cc3)ccc1-2 ; Cc1cc(N(c2ccc(C3CC4CCC3C4)cc2)c2ccccc2-c2ccc(-c3ccccc3-c3ccccc3)cc2)cc2c1-c1ccccc1C2 ; c1ccc(-c2ccccc2-c2ccc(-c3ccccc3N(c3ccc(C4CC5CCC4C5)cc3)c3cc4c(c(-c5ccccc5)c3)-c3ccccc3C4)cc2)cc1 ; Cc1cccc2c1-c1ccc(N(c3ccc(C4CC5CCC4C5)cc3)c3ccccc3-c3ccc(-c4ccccc4-c4ccccc4)cc3)cc1C2C] 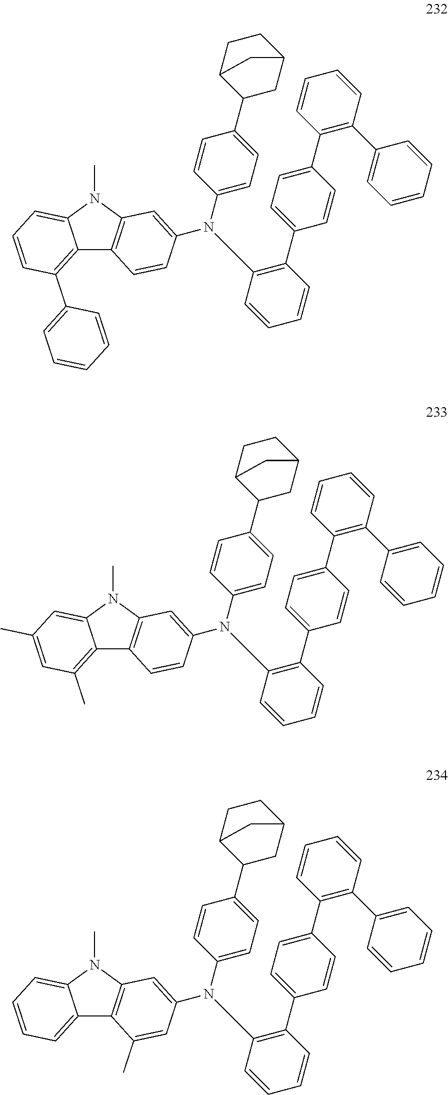 [SMILES: Cn1c2cc(N(c3ccc(C4CC5CCC4C5)cc3)c3ccccc3-c3ccc(-c4ccccc4-c4ccccc4)cc3)ccc2c2c(-c3ccccc3)cccc21 ; Cc1cc(C)c2c3ccc(N(c4ccc(C5CC6CCC5C6)cc4)c4ccccc4-c4ccc(-c5ccccc5-c5ccccc5)cc4)cc3n(C)c2c1 ; Cc1cc(N(c2ccc(C3CC4CCC3C4)cc2)c2ccccc2-c2ccc(-c3ccccc3-c3ccccc3)cc2)cc2c1c1ccccc1n2C] 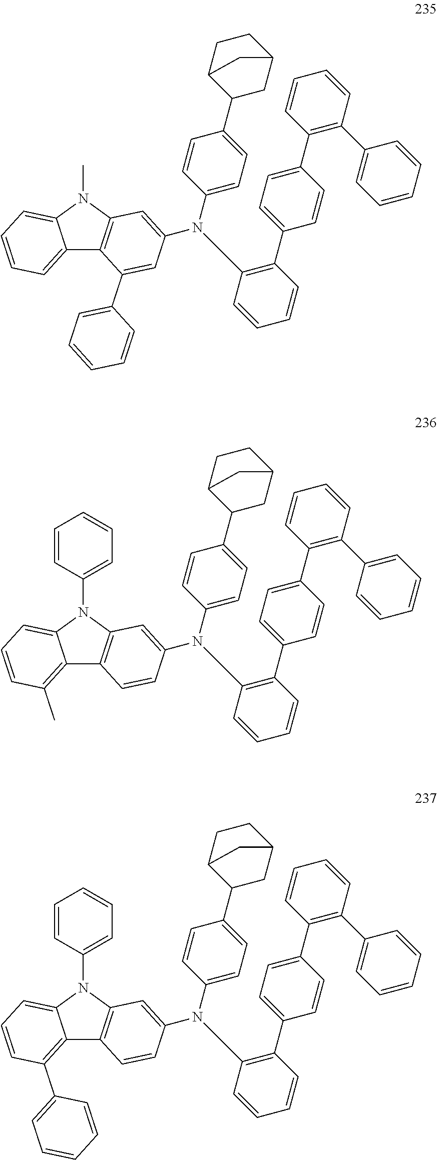 [SMILES: Cn1c2ccccc2c2c(-c3ccccc3)cc(N(c3ccc(C4CC5CCC4C5)cc3)c3ccccc3-c3ccc(-c4ccccc4-c4ccccc4)cc3)cc21 ; Cc1cccc2c1c1ccc(N(c3ccc(C4CC5CCC4C5)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C4CC5CCC4C5)cc3)c3ccc4c5c(-c6ccccc6)cccc5n(-c5ccccc5)c4c3)cc2)cc1] 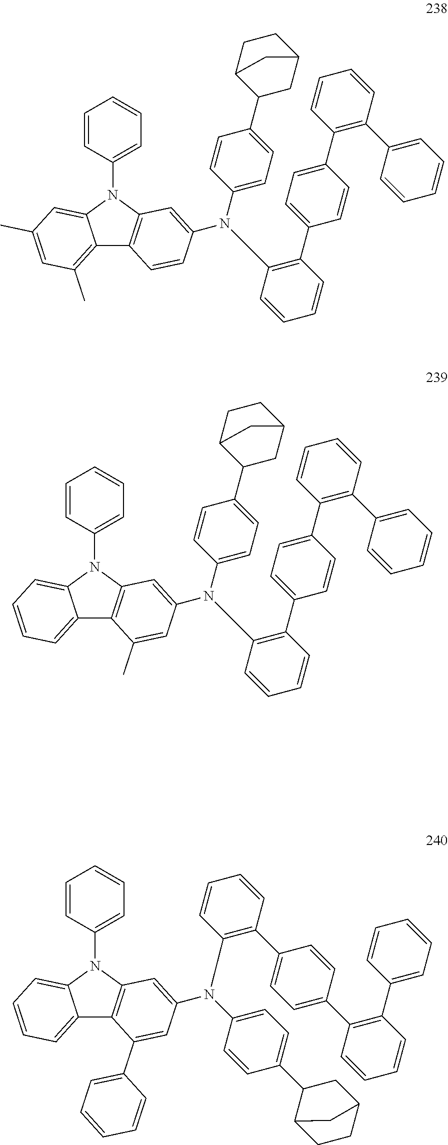 [SMILES: Cc1cc(C)c2c3ccc(N(c4ccc(C5CC6CCC5C6)cc4)c4ccccc4-c4ccc(-c5ccccc5-c5ccccc5)cc4)cc3n(-c3ccccc3)c2c1 ; Cc1cc(N(c2ccc(C3CC4CCC3C4)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C4CC5CCC4C5)cc3)c3cc(-c4ccccc4)c4c5ccccc5n(-c5ccccc5)c4c3)cc2)cc1] 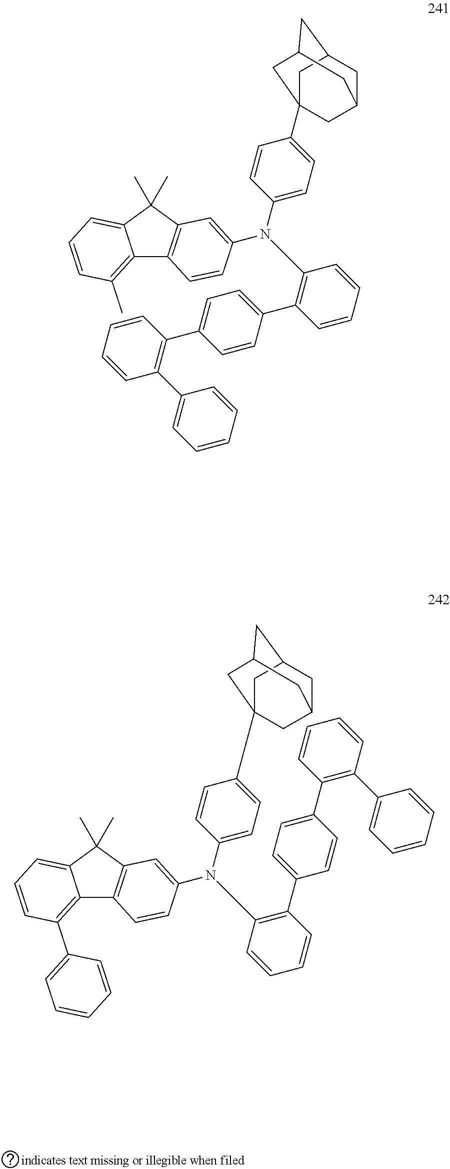 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(C)C ; CC1(C)c2cc(N(c3ccc(C45CC6CC(CC(C6)C4)C5)cc3)c3ccccc3-c3ccc(-c4ccccc4-c4ccccc4)cc3)ccc2-c2c(-c3ccccc3)cccc21] 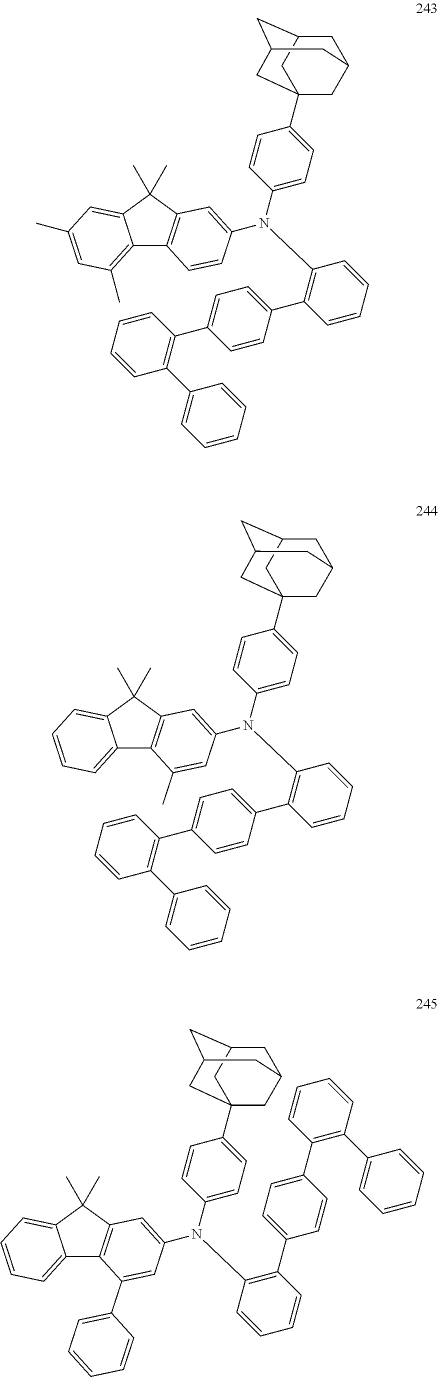 [SMILES: Cc1cc(C)c2c(c1)C(C)(C)c1cc(N(c3ccc(C45CC6CC(CC(C6)C4)C5)cc3)c3ccccc3-c3ccc(-c4ccccc4-c4ccccc4)cc3)ccc1-2 ; Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(C)C ; CC1(C)c2ccccc2-c2c(-c3ccccc3)cc(N(c3ccc(C45CC6CC(CC(C6)C4)C5)cc3)c3ccccc3-c3ccc(-c4ccccc4-c4ccccc4)cc3)cc21] 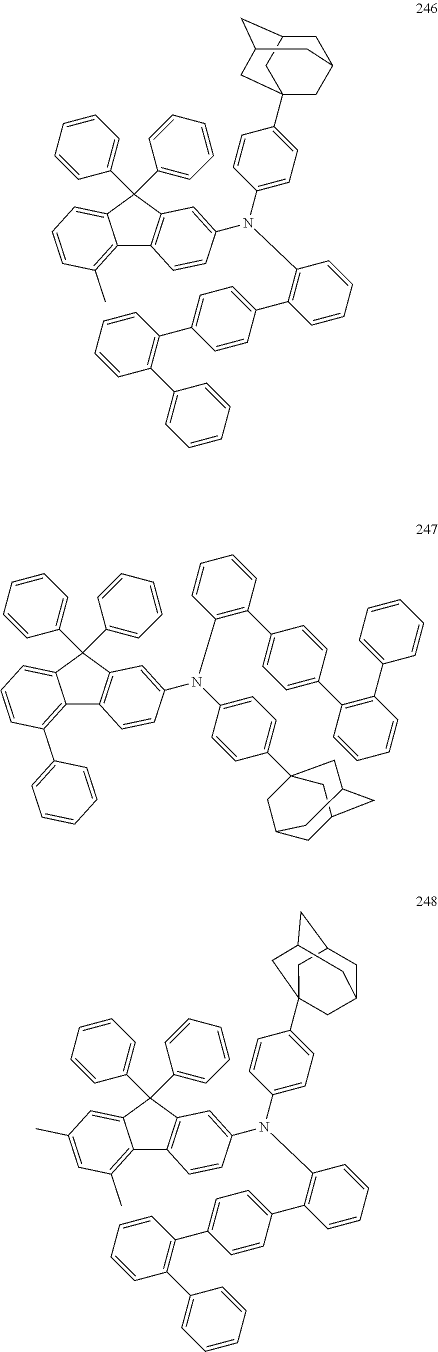 [SMILES: Cc1cccc2c1-c1ccc(N(c3ccc(C45CC6CC(CC(C6)C4)C5)cc3)c3ccccc3-c3ccc(-c4ccccc4-c4ccccc4)cc3)cc1C2(c1ccccc1)c1ccccc1 ; c1ccc(-c2ccccc2-c2ccc(-c3ccccc3N(c3ccc(C45CC6CC(CC(C6)C4)C5)cc3)c3ccc4c(c3)C(c3ccccc3)(c3ccccc3)c3cccc(-c5ccccc5)c3-4)cc2)cc1 ; Cc1cc(C)c2c(c1)C(c1ccccc1)(c1ccccc1)c1cc(N(c3ccc(C45CC6CC(CC(C6)C4)C5)cc3)c3ccccc3-c3ccc(-c4ccccc4-c4ccccc4)cc3)ccc1-2] 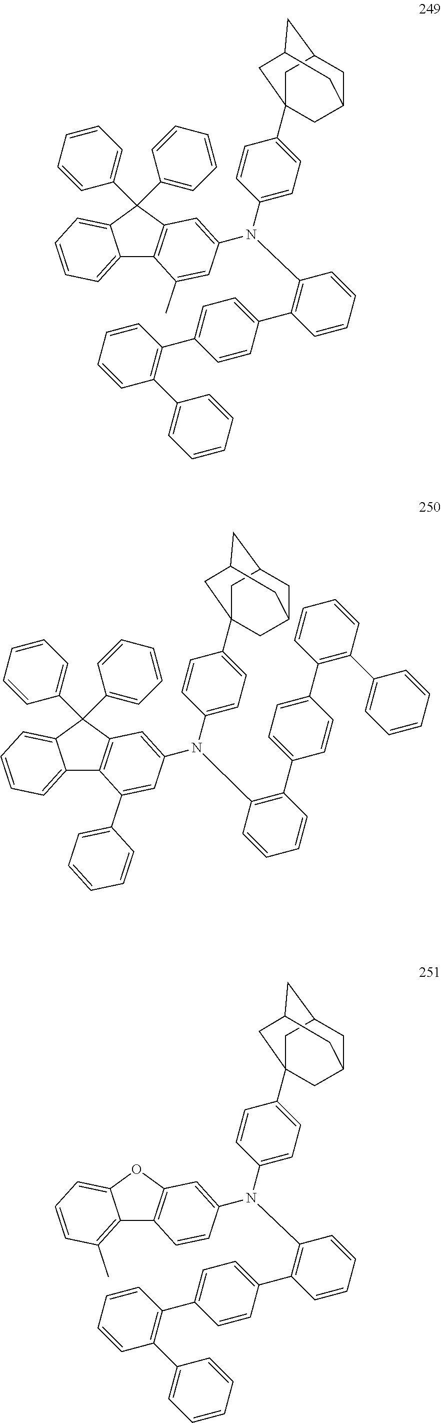 [SMILES: Cc1cc(N(c2ccc(C34CC5CC(CC(C5)C3)C4)cc2)c2ccccc2-c2ccc(-c3ccccc3-c3ccccc3)cc2)cc2c1-c1ccccc1C2(c1ccccc1)c1ccccc1 ; c1ccc(-c2ccccc2-c2ccc(-c3ccccc3N(c3ccc(C45CC6CC(CC(C6)C4)C5)cc3)c3cc(-c4ccccc4)c4c(c3)C(c3ccccc3)(c3ccccc3)c3ccccc3-4)cc2)cc1 ; Cc1cccc2oc3cc(N(c4ccc(C56CC7CC(CC(C7)C5)C6)cc4)c4ccccc4-c4ccc(-c5ccccc5-c5ccccc5)cc4)ccc3c12] 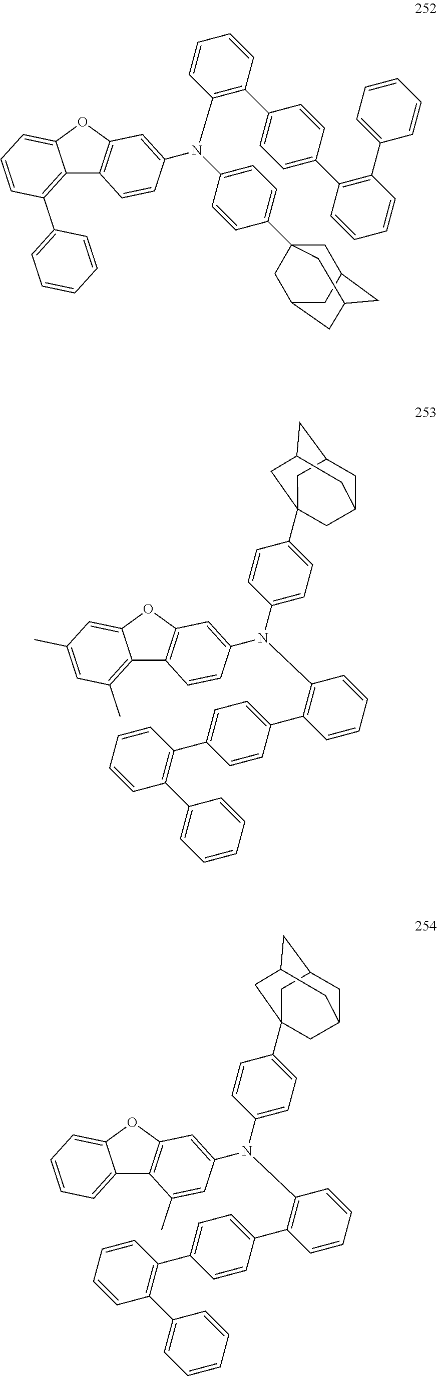 [SMILES: c1ccc(-c2ccccc2-c2ccc(-c3ccccc3N(c3ccc(C45CC6CC(CC(C6)C4)C5)cc3)c3ccc4c(c3)oc3cccc(-c5ccccc5)c34)cc2)cc1 ; Cc1cc(C)c2c(c1)oc1cc(N(c3ccc(C45CC6CC(CC(C6)C4)C5)cc3)c3ccccc3-c3ccc(-c4ccccc4-c4ccccc4)cc3)ccc12 ; Cc1cc(N(c2ccc(C34CC5CC(CC(C5)C3)C4)cc2)c2ccccc2-c2ccc(-c3ccccc3-c3ccccc3)cc2)cc2oc3ccccc3c12] 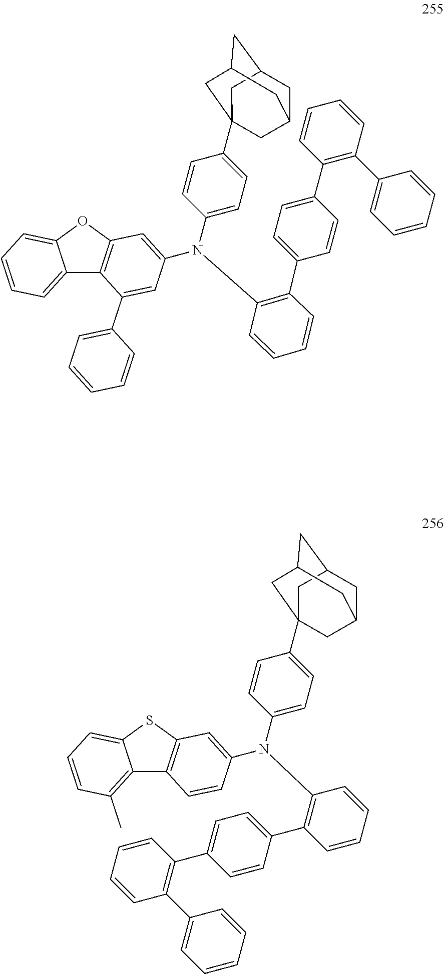 [SMILES: c1ccc(-c2ccccc2-c2ccc(-c3ccccc3N(c3ccc(C45CC6CC(CC(C6)C4)C5)cc3)c3cc(-c4ccccc4)c4c(c3)oc3ccccc34)cc2)cc1 ; Cc1cccc2sc3cc(N(c4ccc(C56CC7CC(CC(C7)C5)C6)cc4)c4ccccc4-c4ccc(-c5ccccc5-c5ccccc5)cc4)ccc3c12] 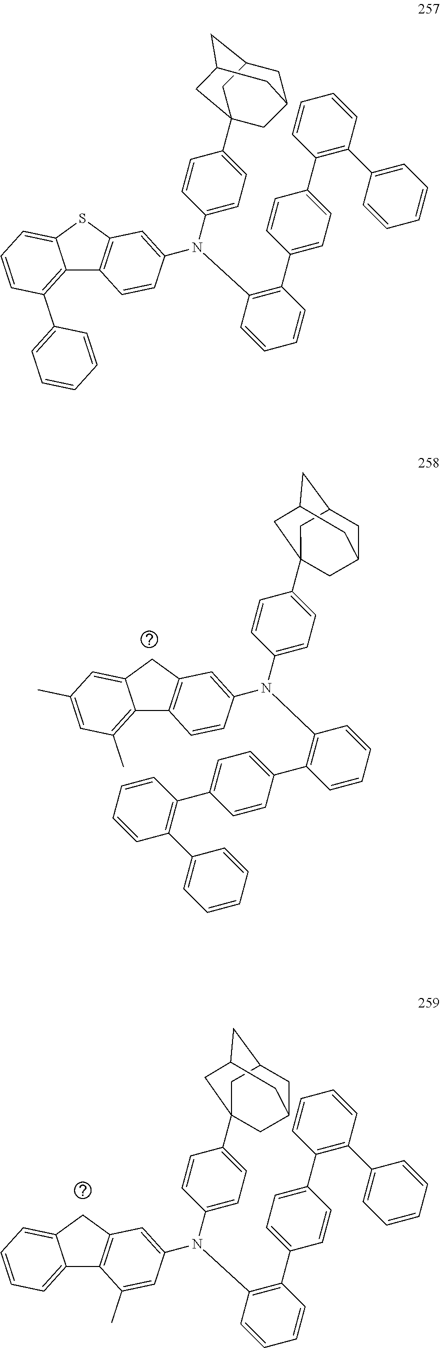 [SMILES: c1ccc(-c2ccccc2-c2ccc(-c3ccccc3N(c3ccc(C45CC6CC(CC(C6)C4)C5)cc3)c3ccc4c(c3)sc3cccc(-c5ccccc5)c34)cc2)cc1 ; Cc1cc(C)c2c(c1)Cc1cc(N(c3ccc(C45CC6CC(CC(C6)C4)C5)cc3)c3ccccc3-c3ccc(-c4ccccc4-c4ccccc4)cc3)ccc1-2 ; Cc1cc(N(c2ccc(C34CC5CC(CC(C5)C3)C4)cc2)c2ccccc2-c2ccc(-c3ccccc3-c3ccccc3)cc2)cc2c1-c1ccccc1C2] 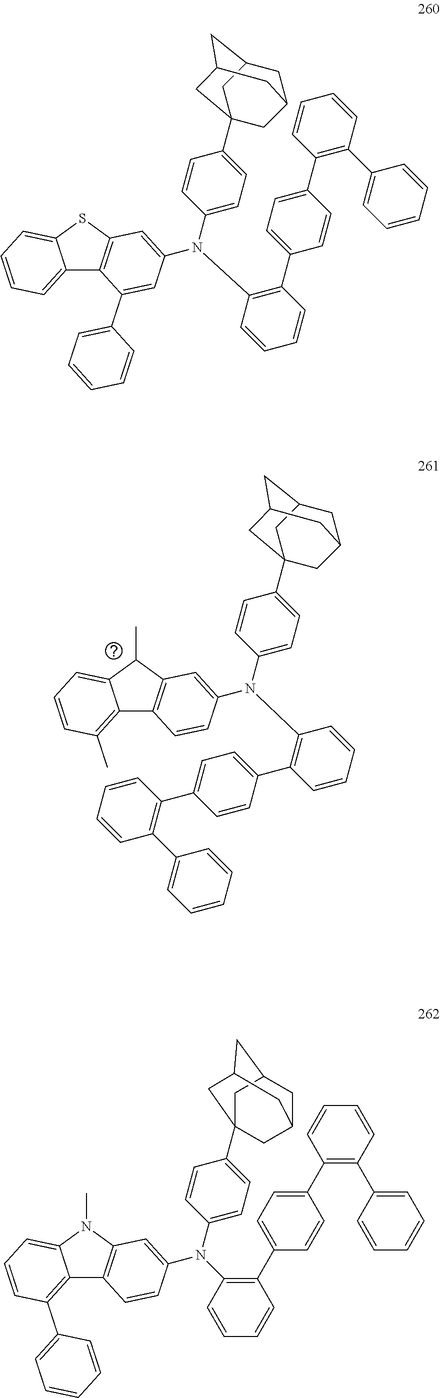 [SMILES: c1ccc(-c2ccccc2-c2ccc(-c3ccccc3N(c3ccc(C45CC6CC(CC(C6)C4)C5)cc3)c3cc(-c4ccccc4)c4c(c3)sc3ccccc34)cc2)cc1 ; Cc1cccc2c1-c1ccc(N(c3ccc(C45CC6CC(CC(C6)C4)C5)cc3)c3ccccc3-c3ccc(-c4ccccc4-c4ccccc4)cc3)cc1C2C ; Cn1c2cc(N(c3ccc(C45CC6CC(CC(C6)C4)C5)cc3)c3ccccc3-c3ccc(-c4ccccc4-c4ccccc4)cc3)ccc2c2c(-c3ccccc3)cccc21] 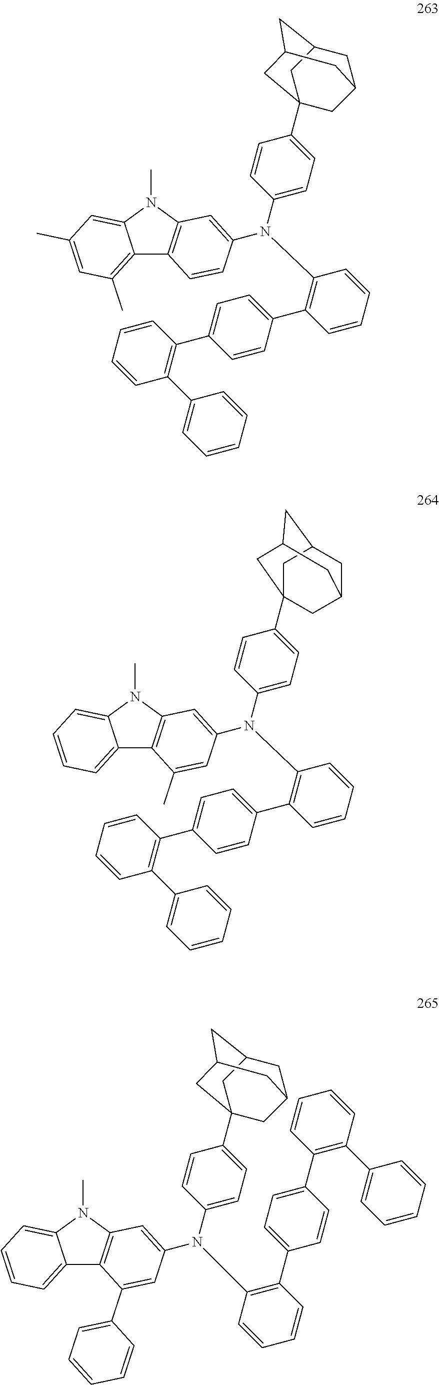 [SMILES: Cc1cc(C)c2c3ccc(N(c4ccc(C56CC7CC(CC(C7)C5)C6)cc4)c4ccccc4-c4ccc(-c5ccccc5-c5ccccc5)cc4)cc3n(C)c2c1 ; Cc1cc(N(c2ccc(C34CC5CC(CC(C5)C3)C4)cc2)c2ccccc2-c2ccc(-c3ccccc3-c3ccccc3)cc2)cc2c1c1ccccc1n2C ; Cn1c2ccccc2c2c(-c3ccccc3)cc(N(c3ccc(C45CC6CC(CC(C6)C4)C5)cc3)c3ccccc3-c3ccc(-c4ccccc4-c4ccccc4)cc3)cc21] 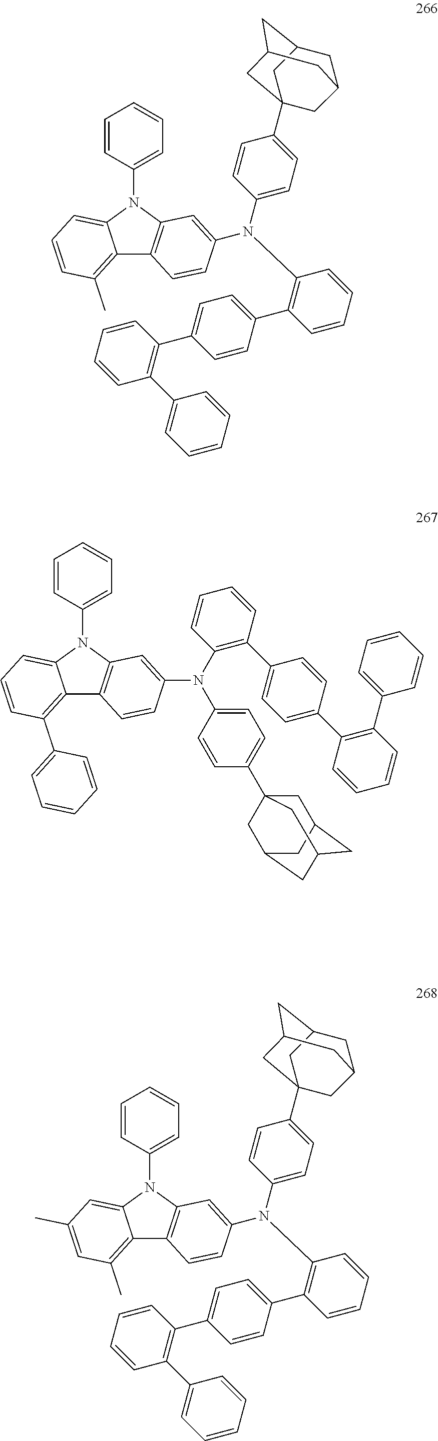 [SMILES: Cc1cccc2c1c1ccc(N(c3ccc(C45CC6CC(CC(C6)C4)C5)cc3)c3ccccc3-c3ccc(-c4ccccc4-c4ccccc4)cc3)cc1n2-c1ccccc1 ; c1ccc(-c2ccccc2-c2ccc(-c3ccccc3N(c3ccc(C45CC6CC(CC(C6)C4)C5)cc3)c3ccc4c5c(-c6ccccc6)cccc5n(-c5ccccc5)c4c3)cc2)cc1 ; Cc1cc(C)c2c3ccc(N(c4ccc(C56CC7CC(CC(C7)C5)C6)cc4)c4ccccc4-c4ccc(-c5ccccc5-c5ccccc5)cc4)cc3n(-c3ccccc3)c2c1] 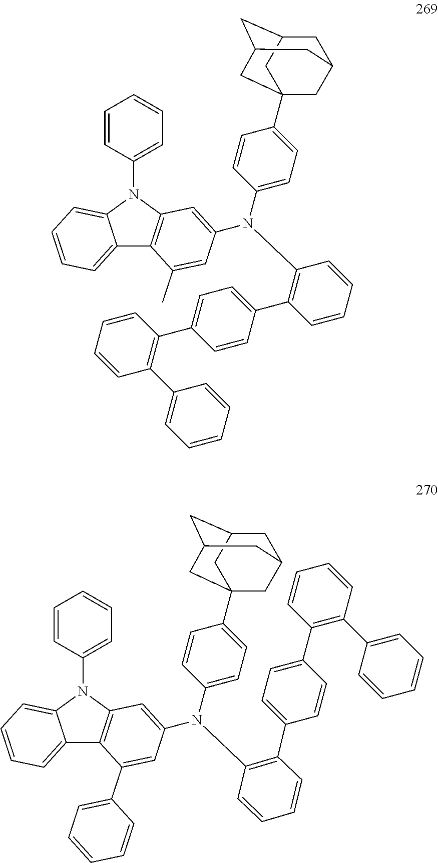 [SMILES: Cc1cc(N(c2ccc(C34CC5CC(CC(C5)C3)C4)cc2)c2ccccc2-c2ccc(-c3ccccc3-c3ccccc3)cc2)cc2c1c1ccccc1n2-c1ccccc1 ; c1ccc(-c2ccccc2-c2ccc(-c3ccccc3N(c3ccc(C45CC6CC(CC(C6)C4)C5)cc3)c3cc(-c4ccccc4)c4c5ccccc5n(-c5ccccc5)c4c3)cc2)cc1] 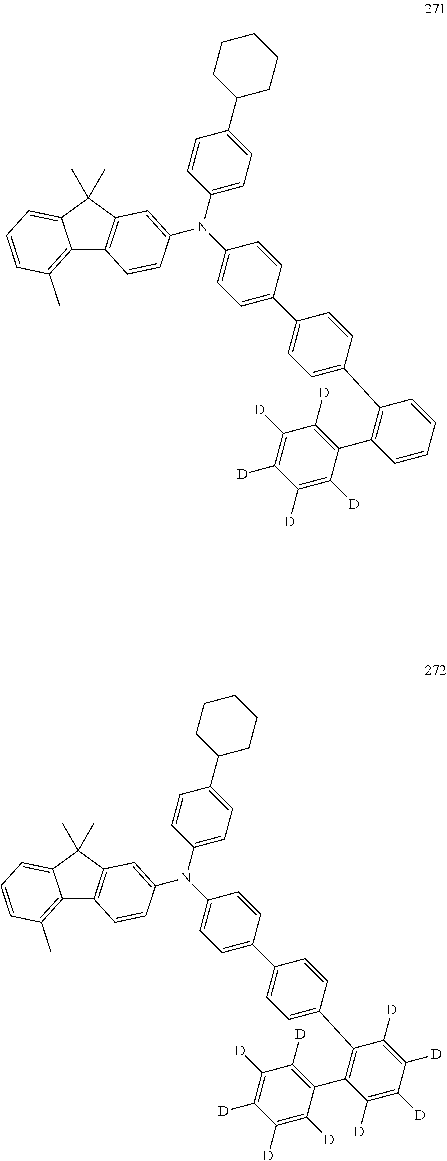 [SMILES: [2H]c1c([2H])c([2H])c(-c2ccccc2-c2ccc(-c3ccc(N(c4ccc(C5CCCCC5)cc4)c4ccc5c(c4)C(C)(C)c4cccc(C)c4-5)cc3)cc2)c([2H])c1[2H] ; [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H]] 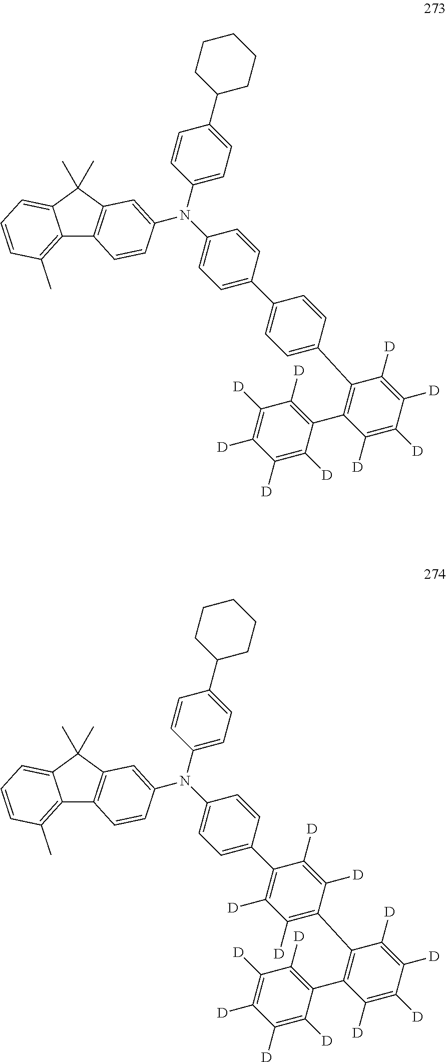 [SMILES: [2H]c1c([2H])c([2H])c(-c2c([2H])c([2H])c([2H])c([2H])c2-c2ccc(-c3ccc(N(c4ccc(C5CCCCC5)cc4)c4ccc5c(c4)C(C)(C)c4cccc(C)c4-5)cc3)cc2)c([2H])c1[2H] ; [2H]c1c([2H])c([2H])c(-c2c([2H])c([2H])c([2H])c([2H])c2-c2c([2H])c([2H])c(-c3ccc(N(c4ccc(C5CCCCC5)cc4)c4ccc5c(c4)C(C)(C)c4cccc(C)c4-5)cc3)c([2H])c2[2H])c([2H])c1[2H]] 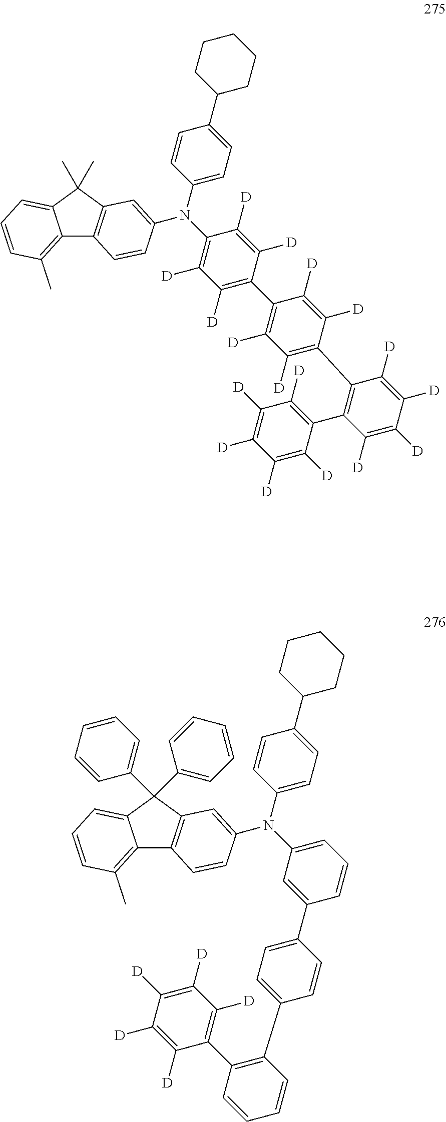 [SMILES: [2H]c1c([2H])c([2H])c(-c2c([2H])c([2H])c([2H])c([2H])c2-c2c([2H])c([2H])c(-c3c([2H])c([2H])c(N(c4ccc(C5CCCCC5)cc4)c4ccc5c(c4)C(C)(C)c4cccc(C)c4-5)c([2H])c3[2H])c([2H])c2[2H])c([2H])c1[2H] ; [2H]c1c([2H])c([2H])c(-c2ccccc2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H]] 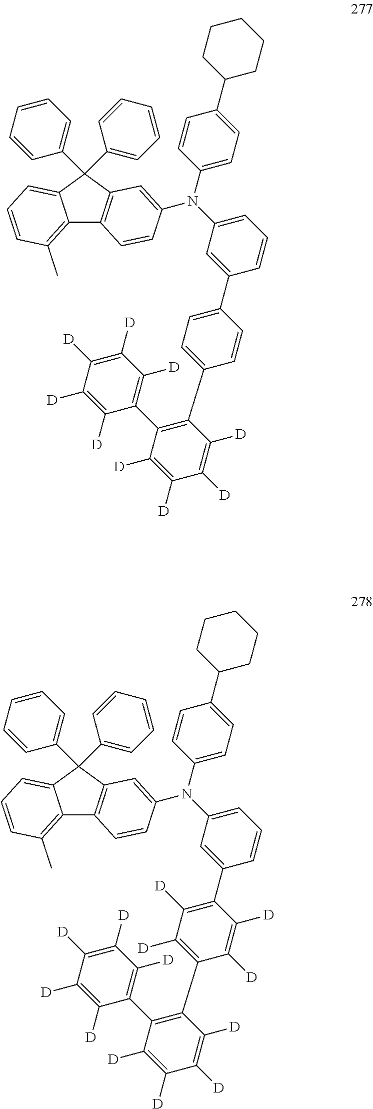 [SMILES: [2H]c1c([2H])c([2H])c(-c2c([2H])c([2H])c([2H])c([2H])c2-c2ccc(-c3cccc(N(c4ccc(C5CCCCC5)cc4)c4ccc5c(c4)C(c4ccccc4)(c4ccccc4)c4cccc(C)c4-5)c3)cc2)c([2H])c1[2H] ; [2H]c1c([2H])c([2H])c(-c2c([2H])c([2H])c([2H])c([2H])c2-c2c([2H])c([2H])c(-c3cccc(N(c4ccc(C5CCCCC5)cc4)c4ccc5c(c4)C(c4ccccc4)(c4ccccc4)c4cccc(C)c4-5)c3)c([2H])c2[2H])c([2H])c1[2H]] 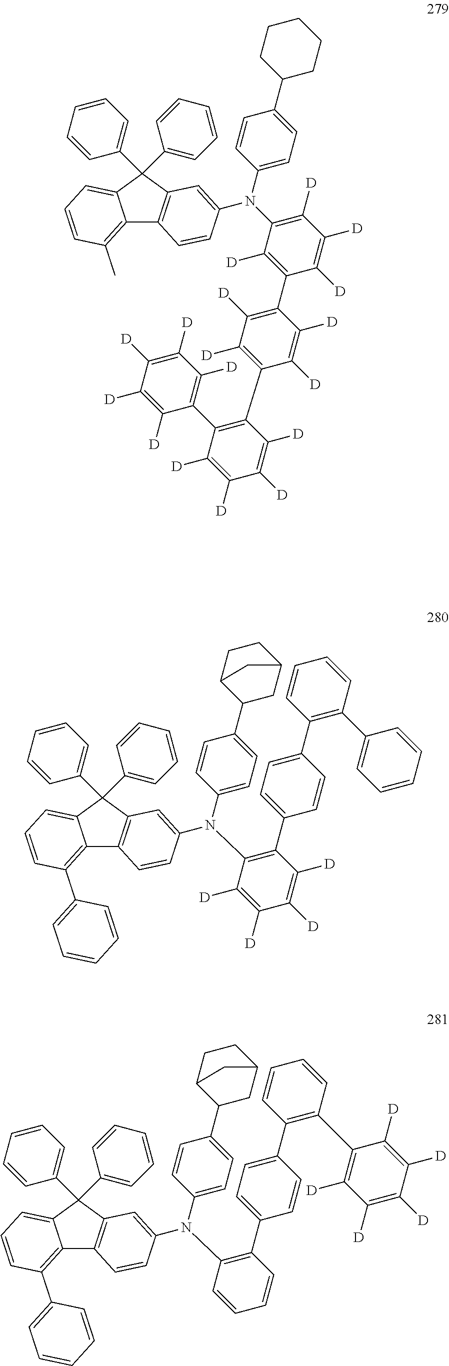 [SMILES: [2H]c1c([2H])c([2H])c(-c2c([2H])c([2H])c([2H])c([2H])c2-c2c([2H])c([2H])c(-c3c([2H])c([2H])c([2H])c(N(c4ccc(C5CCCCC5)cc4)c4ccc5c(c4)C(c4ccccc4)(c4ccccc4)c4cccc(C)c4-5)c3[2H])c([2H])c2[2H])c([2H])c1[2H] ; [2H]c1c([2H])c([2H])c(N(c2ccc(C3CC4CCC3C4)cc2)c2ccc3c(c2)C(c2ccccc2)(c2ccccc2)c2cccc(-c4ccccc4)c2-3)c(-c2ccc(-c3ccccc3-c3ccccc3)cc2)c1[2H] ; [2H]c1c([2H])c([2H])c(-c2ccccc2-c2ccc(-c3ccccc3N(c3ccc(C4CC5CCC4C5)cc3)c3ccc4c(c3)C(c3ccccc3)(c3ccccc3)c3cccc(-c5ccccc5)c3-4)cc2)c([2H])c1[2H]] [chemical structure]
